(19) United States
(12) Patent Application Publication
(10) Pub. No.: US 2025/0325702 A1
(43) Pub. Date: Oct. 23, 2025

(54) COMPOSITIONS AND METHODS FOR EDITING A TRANSTHYRETIN GENE

(71) Applicant: Beam Therapeutics Inc., Cambridge, MA (US)

(72) Inventors: Genesis Lung, Cambridge, MA (US); Lo-I Cheng, Cambridge, MA (US); Tanggis Bohnuud, Cambridge, MA (US); Cory D. Sago, Cambridge, MA (US)

(73) Assignee: Beam Therapeutics Inc., Cambridge, MA (US)

(21) Appl. No.: 19/209,589

(22) Filed: May 15, 2025

Related U.S. Application Data
(63) Continuation of application No. PCT/US2023/080721, filed on Nov. 21, 2023.
(60) Provisional application No. 63/385,004, filed on Nov. 25, 2022.

(51) Int. Cl. A61K 48/00; A61K 9/1271; A61K 9/51; A61P 3/00; C12N 9/22; C12N 15/11; C12N 15/78; C12N 15/88

(52) U.S. Cl. CPC A61K 48/005; A61K 9/1271; A61K 9/5123; A61K 48/0075; A61P 3/00; C12N 9/226; C12N 15/111; C12N 15/78; C12N 15/88; C07K 2319/80; C12N 2310/20; C12N 2310/315; C12N 2310/321; C12Y 305/04004

(57) ABSTRACT

Compositions for gene modification related to base editor systems, and methods of using the same to treat or prevent conditions associated with the extracellular deposition in various tissues of amyloid fibrils formed by the aggregation of misfolded transthyretin (TTR) proteins. Such conditions include, but are not limited to, polyneuropathy due to hereditary transthyretin amyloidosis (hATTR-PN) and hereditary cardiomyopathy due to transthyretin amyloidosis (hATTR-CM), both associated with autosomal dominant mutations of the TTR gene, and an age-related cardiomyopathy associated with wild-type TTR proteins (ATTRwt), also known as senile cardiac amyloidosis.

### CROSS REFERENCE TO RELATED APPLICATIONS

This application is a continuation under 35 U.S.C. § 111 (a) of PCT International Patent Application No. PCT/US2023/080721, filed Nov. 21, 2023, designating the United States and published in English, which claims priority to and the benefit of U.S. Provisional Application No. 63/385,004, filed Nov. 25, 2022, the entire contents of each of which are incorporated by reference herein.

### SEQUENCE LISTING

This application contains a Sequence Listing which has been submitted electronically in XML format and is hereby incorporated by reference in its entirety. The Sequence Listing XML file, created on Nov. 21, 2023, is named 180802-049303US_SL.xml and is 1,593,694 bytes in size.

### BACKGROUND

Amyloidosis is a condition characterized by the buildup of abnormal deposits of amyloid protein in the body's organs and tissues. These protein deposits can occur in the peripheral nervous system, which is made up of nerves connecting the brain and spinal cord to muscles and sensory cells that detect sensations such as touch, pain, heat, and sound. Protein deposits in these nerves can result in a loss of sensation in the extremities (peripheral neuropathy). The autonomic nervous system, which controls involuntary body functions, such as blood pressure, heart rate, and digestion, can also be affected by amyloidosis. In some cases, the brain and spinal cord (central nervous system) are affected. Mutations in the transthyretin (TTR) gene can cause transthyretin amyloidosis. Furthermore, patients expressing wild-type TTR may also develop amyloidosis. Liver transplant remains the gold standard for treating transthyretin amyloidosis. However, there are a limited number of organ donors, and patients may wait years for an available organ. Accordingly, there is a need for compositions and methods for treating amyloidosis.

### SUMMARY

Provided herein are compositions for gene modification or editing and methods of using the same to treat or prevent conditions associated with the extracellular deposition in various tissues of amyloid fibrils formed by the aggregation of misfolded transthyretin (TTR) proteins. Such conditions include, but are not limited to, polyneuropathy due to hereditary transthyretin amyloidosis (hATTR-PN) and hereditary cardiomyopathy due to transthyretin amyloidosis (hATTR-CM), both associated with autosomal dominant mutations of the TTR gene, and an age-related cardiomyopathy associated with wild-type TTR proteins (ATTRwt), also known as senile cardiac amyloidosis. Compositions and methods directed to editing the TTR gene using an editing system, such as one comprising a base editor and guide RNAs are disclosed.

In one aspect, the disclosure features a lipid nanoparticle (LNP) containing a guide polynucleotide containing a sequence selected from any one or more of the following SEQ ID NOs: 472-476, 479-497, 499-504, 506-532, 534-571, 573-638, 653-677, 707-711, 713-731, 733-784, 1044, 1045, 1214, and 1215, and/or any sequence provided in the sequence listing submitted herewith. The guide polynucleotide does not contain the sequence GCCAUCCUGCCAAGAAUGAG (SEQ ID NO: 472). The lipid nanoparticle contains an amino lipid according to any one of the following Formulas:

A) an amino lipid of Formula (Ia):

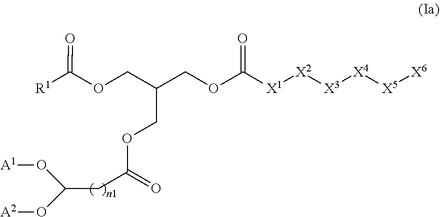

where:
R¹ is C₉-C₂₀ alkyl or C₉-C₂₀ alkenyl with 1-3 units of unsaturation;
X¹ and X² are each independently absent or selected from —O—, —NR₂— and

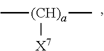

where each R² is independently hydrogen or C₁-C₆ alkyl;
each a is independently an integer between 1 and 6;
X³ and X⁴ are each independently absent or selected from one or more of: 4- to 8-membered heterocyclyl optionally substituted with 1 or 2 C₁-C₆ alkyl groups, 5- to 6-membered heteroaryl optionally substituted with 1 or 2 C₁-C₆ alkyl groups, 5- to 6-membered aryl optionally substituted with 1 or 2 C₁-C₆ alkyl groups, 4- to 7-membered cycloalkyl optionally substituted with 1 or 2 C₁-C₆ alkyl groups, —O— and —NR³—, where each R³ is a independently a hydrogen atom or C₁-C₆ alkyl and where X¹-X²-X³-X⁴ does not contain any oxygen-oxygen, oxygen-nitrogen or nitrogen-nitrogen bonds;
X⁵ is —(CH₂)_{b}—, where b is an integer between 0 and 6;
X⁶ is hydrogen, C₁-C₆ alkyl, 5- to 6-membered heteroaryl optionally substituted with 1 or 2 C₁-C₆ alkyl groups, or —NR⁴R⁵, where R⁴ and R⁵ are each independently hydrogen or C₁-C₆ alkyl; or alternatively R⁴ and R⁵ join together with the nitrogen to which they are bound to form a 4- to 7-membered heterocyclyl optionally substituted with 1 or 2 C₁-C₆ alkyl groups, where the heterocyclyl optionally includes an additional heteroatom selected from oxygen, sulfur, and nitrogen;
each X⁷ is independently hydrogen, hydroxyl or —NR⁶R⁷, where R⁶ and R⁷ are each independently hydrogen or C₁-C₆ alkyl; or alternatively R⁶ and R⁷ join together with the nitrogen to which they are bound to form a 4- to 7-membered heterocyclyl optionally substituted with 1 or 2 C₁-C₆ alkyl groups, where the heterocyclyl optionally includes an additional heteroatom selected from oxygen, sulfur, and nitrogen;
at least one of X¹, X², X³, X⁴, and X⁵ is present;
A¹ and A² are each independently selected from one or more of: C₅-C₁₂ haloalkyl, C₅-C₁₂ alkenyl, C₅-C₁₂ alkynyl, (C₅-C₁₂ alkoxy)-(CH₂)ₙ₂—, (C₅-C₁₀ aryl)-(CH₂)ₙ₃— optionally ring substituted with one or two halo, C₁-C₆ alkyl, C₁-C₆ haloalkyl, or C₁-C₆ alkoxy groups, and (C₃-C₈ cycloalkyl)-(CH₂)ₙ₄-optionally ring substituted with 1 or 2 C₁-C₆ alkyl groups; or alternatively A¹ and A² join together with the atoms to which they are bound to form a 5- to 6-membered cyclic acetal substituted with 1 or 2 C₄-C₁₀ alkyl groups;
n1, n2 and n3 are each individually an integer between 1 and 4; and
n4 is an integer between zero and 4;
   B) an amino lipid of Formula (Ib):

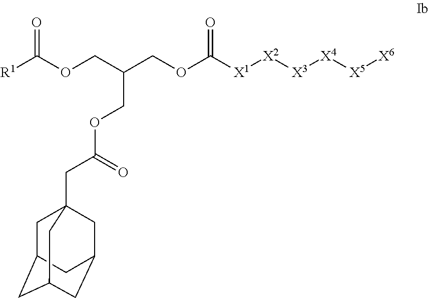

where:
R¹ is C₉-C₂₀ alkyl or C₉-C₂₀ alkenyl with 1-3 units of unsaturation;
X¹ and X² are each independently absent or selected from —O—, NR², and

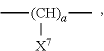

where R² is C₁-C₆ alkyl, and where X¹ and X² are not both —O— or NR²;
a is an integer between 1 and 6;
X³ and X⁴ are each independently absent or selected from one or more of: 4- to 7-membered heterocyclyl optionally substituted with 1 or 2 C₁-C₆ alkyl groups, 5- to 6-membered heteroaryl optionally substituted with 1 or 2 C₁-C₆ alkyl groups, and —NR³—, where each R³ is a hydrogen atom or C₁-C₆ alkyl;
X⁵ is —(CH₂)_{b}—, where b is an integer between 0 and 6;
X⁶ is hydrogen, C₁-C₆ alkyl, 5- to 6-membered heteroaryl optionally substituted with 1 or 2 C₁-C₆ alkyl groups, or —NR⁴R⁵, where R⁴ and R⁵ are each independently hydrogen or C₁-C₆ alkyl; or alternatively R⁴ and R⁵ join together with the nitrogen to which they are bound to form a 4- to 7-membered heterocyclyl optionally substituted with 1 or 2 C₁-C₆ alkyl groups, where the heterocyclyl optionally includes an additional heteroatom selected from oxygen, sulfur, and nitrogen;
X⁷ is hydrogen or —NR⁶R⁷, where R⁶ and R⁷ are each independently hydrogen or C₁-C₆ alkyl; or alternatively R⁶ and R⁷ join together with the nitrogen to which they are bound to form a 4- to 7-membered heterocyclyl optionally substituted with 1 or 2 C₁-C₆ alkyl groups, where the heterocyclyl optionally includes an additional heteroatom selected from oxygen, sulfur, and nitrogen;
at least one of X¹, X², X³, X⁴, and X⁵ is present; and
provided that when either X¹ or X² is —O—, neither X³ nor X⁴ is

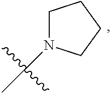 [SMILES: *N1CCCC1]

and when either X¹ or X² is —O—, R⁴ and R⁵ are not both ethyl;
C) an amino lipid of Formula (Ic):

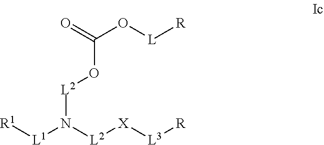

or its N-oxide, or a salt thereof, where
   L1 is C₁-6 alkylenyl, or C₂-6 heteroalkylenyl;
each L² is independently C₂-10 alkylenyl, or C₃-10 heteroalkylenyl;
L is absent, C₁-10 alkylenyl, or C₂-10 heteroalkylenyl;
L³ is absent, C₁-10 alkylenyl, or C₂-10 heteroalkylenyl;
X is absent, —OC(O)—, —C(O)O—, or —OC(O)O—;
each R is independently hydrogen,

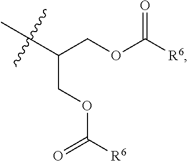

or an optionally substituted group selected from C₆-20 aliphatic, C₆-20 haloaliphatic, a 3- to 7-membered cycloaliphatic ring, 1-adamantyl, 2-adamantyl, sterolyl, and phenyl;
R¹ is hydrogen, a 3- to 7-membered cycloaliphatic ring, a 3- to 7-membered heterocyclic ring containing 1-3 heteroatoms independently selected from nitrogen, oxygen, and sulfur, —OR², —C(O)OR², —C(O)SR², —OC(O)R², —OC(O)OR², —CN, —N(R²)₂, —C(O)N(R²)₂, —NR²C(O)R², —OC(O)N(R²)₂, —N(R²)C(O)OR², —NR²S(O)₂R², —NR²C(O)N(R²)₂, —NR²C(S)N(R²)₂, —NR²C(NR²)N(R²)₂, —NR²C(CHR²)N(R²)₂, —N(OR²)C(O)R², —N(OR²)S(O)₂R², —N(OR²)C(O)OR², —N(OR²)C(O)N(R²)₂, —N(OR²)C(S)N(R²)₂, —N(OR²)C(NR²)N(R²)₂, —N(OR²)C(CHR²)N(R²)₂, —C(NR²)N(R²)₂, —C(NR²)R², —C(O)N(R²)OR², —C(R²)N(R²)₂C(O)OR²,

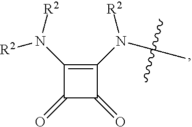

—CR²(OR²)R³,

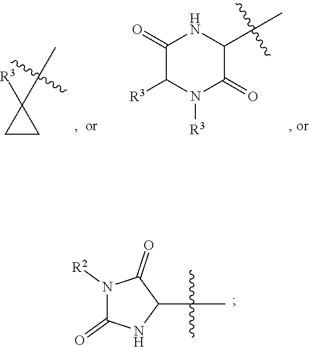

each R² is independently hydrogen, —CN, —NO₂, —OR⁴, —S(O)₂R⁴, —S(O)₂N(R⁴)₂, —(CH₂)ₙ—R⁴, or an optionally substituted group selected from C₁-6 aliphatic, a 3- to 7-membered cycloaliphatic ring, and a 3- to 7-membered heterocyclic ring containing 1-3 heteroatoms independently selected from nitrogen, oxygen, and sulfur, or
   two occurrences of R², taken together with the atom(s) to which they are attached, form an optionally substituted 4- to 7-membered heterocyclic ring containing 0-1 additional heteroatom selected from nitrogen, oxygen, and sulfur;
each R³ is independently —(CH₂)ₙ—R⁴, or
   two occurrences of R³, taken together with the atoms to which they are attached, form an optionally substituted 5- to 6-membered heterocyclic ring containing 0-1 additional heteroatom selected from nitrogen, oxygen, and sulfur;
each R⁴ is independently
hydrogen, —OR⁵, —N(R⁵)₂, —OC(O)R⁵, —OC(O)OR⁵, —CN, —C(O)N(R⁵)₂, —NR⁵C(O)R⁵, —OC(O)N(R⁵)₂, —N(R⁵)C(O)OR⁵, —NR⁵S(O)₂R⁵, —NR⁵C(O)N(R⁵)₂, —NR⁵C(S)N(R⁵)₂, —NR⁵C(NR⁵)N(R⁵)₂, or

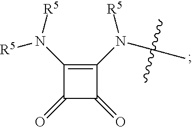

each R⁵ is independently hydrogen, optionally substituted C₁₋₆ aliphatic, or
   two occurrences of R⁵, taken together with the atom(s) to which they are attached, form an optionally substituted 4- to 7-membered heterocyclic ring containing 0-1 additional heteroatom selected from nitrogen, oxygen, and sulfur;
each R⁶ is independently C₄-12 aliphatic; and
   n is 0 to 4;
   D) an amino lipid of Formula (Id):

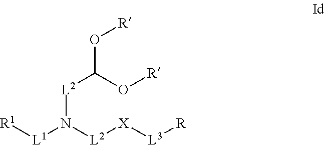

or its N-oxide, or a pharmaceutically acceptable salt thereof, where
L1 is absent, C₁-6 alkylenyl, or C₂-6 heteroalkylenyl;
each L2 is independently optionally substituted C₂₋₁₅ alkylenyl, or optionally substituted C₃₋₁₅ heteroalkylenyl;
L3 is absent, optionally substituted C₁₋₁₀ alkylenyl, or optionally substituted C₂₋₁₀ heteroalkylenyl;
X is absent, —OC(O)—, —C(O)O—, or —OC(O)O—;
each R′ is independently an optionally substituted group selected from C₄-12 aliphatic, 3- to 12-membered cycloaliphatic, 7- to 12-membered bridged bicyclic containing 0-4 heteroatoms independently selected from nitrogen, oxygen, or sulfur, 1-adamantyl, 2-adamantyl, sterolyl, and phenyl;
R is hydrogen,

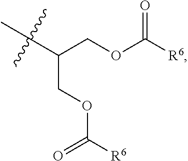

or an optionally substituted group selected from C₆-20 aliphatic, 3- to 12-membered cycloaliphatic, 7- to 12-membered bridged bicyclic containing 0-4 heteroatoms independently selected from nitrogen, oxygen, or sulfur, 1-adamantyl, 2-adamantyl, sterolyl, and phenyl;
R¹ is hydrogen, optionally substituted phenyl, optionally substituted 3- to 7-membered cycloaliphatic, optionally substituted 3- to 7-membered heterocyclyl containing 1-3 heteroatoms independently selected from nitrogen, oxygen, and sulfur, optionally substituted 5- to 6-membered monocyclic heteroaryl containing 1-4 heteroatoms independently selected from nitrogen, oxygen, and sulfur, optionally substituted 8- to 10-membered bicyclic heteroaryl containing 1-4 heteroatoms independently selected from nitrogen, oxygen, and sulfur, —OR², —C(O)OR², —C(O) SR², —OC(O)R², —OC(O)OR², —CN, —N(R²)₂, —C(O)N(R²)₂, —S(O)₂N(R²)₂, —NR²C(O)R², —OC(O)N(R²)₂, —N(R²)C(O)OR², —NR²S(O)₂R², —NR²C(O)N(R²)₂, —NR²C(S)N(R²)₂, —NR²C(NR²)N(R²)₂, —NR²C(CHR²)N(R²)₂, —N(OR²)C(O)R², —N(OR²) S(0)₂R², —N(OR²)C(O)OR², —N(OR²)C(O)N(R²)₂, —N(OR²)C(S)N(R²)₂, —N(OR²)C(NR²)N(R²)₂, —N(OR²)C(CHR²)N(R²)₂, —C(NR²)N(R²)₂, —C(NR²)R², —C(O)N(R²)OR², —C(R²)N(R²)₂C(O)OR², —CR² (R³)₂, —OP(O)(OR²)₂, or —P(O) (OR²)₂; or
R¹ is

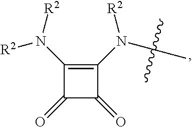

or a ring selected from 3- to 7-membered cycloaliphatic and 3- to 7-membered heterocyclyl containing 1-3 heteroatoms independently selected from nitrogen, oxygen, and sulfur, where the cycloaliphatic or heterocyclyl ring is optionally substituted with 1-4 R² or R³ groups;
each R² is independently hydrogen, oxo, —CN, —NO₂, —OR⁴, —S(O)₂R⁴, —S(O)₂N(R⁴)₂, —(CH₂) n-R⁴, or an optionally substituted group selected from C₁-6 aliphatic, phenyl, 3- to 7-membered cycloaliphatic, 5- to 6-membered monocyclic heteroaryl containing 1-4 heteroatoms independently selected from nitrogen, oxygen, and sulfur, and 3- to 7-membered heterocyclyl containing 1-3 heteroatoms independently selected from nitrogen, oxygen, and sulfur; or
   two occurrences of R², taken together with the atom(s) to which they are attached, form optionally substituted 4- to 7-membered heterocyclyl containing 0-1 additional heteroatom selected from nitrogen, oxygen, and sulfur;
each R³ is independently-(CH₂) n-R⁴; or
   two occurrences of R³, taken together with the atom(s) to which they are attached, form optionally substituted 5- to 6-membered heterocyclyl containing 0-1 additional heteroatom selected from nitrogen, oxygen, and sulfur;
each R⁴ is independently hydrogen, —OR⁵, —N(R⁵)₂, —OC(O)R⁵, —OC(O)OR⁵, —CN, —C(O)N(R⁵)₂,
   —NR⁵C(O)R⁵, —OC(O)N(R⁵)₂, —N(R⁵)C(O)OR⁵, —NR⁵S(O)₂R⁵, —NR⁵C(O)N(R⁵)₂,
   —NR⁵C(S)N(R⁵)₂, —NR⁵° C. (NR⁵)N(R⁵)₂, or

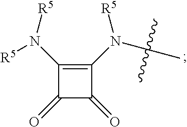

each R⁵ is independently hydrogen, or optionally substituted C₁₋₆ aliphatic; or
   two occurrences of R⁵, taken together with the atom(s) to which they are attached, form optionally substituted 4- to 7-membered heterocyclyl containing 0-1 additional heteroatom selected from nitrogen, oxygen, and sulfur;
each R⁶ is independently C₄₋₁₂ aliphatic; and
each n is independently 0 to 4;
   E) an amino lipid of Formula (Ie):

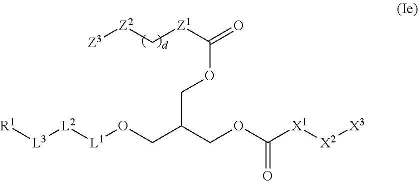

or a pharmaceutically acceptable salt thereof, where:
L1 is a covalent bond, —C(O)—, or —OC(O)—;
L2 is a covalent bond, an optionally substituted bivalent saturated or unsaturated, straight or branched C₁-C₁₂ hydrocarbon chain, or

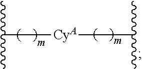

Cy^{A} is an optionally substituted ring selected from phenylene and 3- to 7-membered saturated or partially unsaturated carbocyclene;
each m is independently 0, 1, or 2;
L3 is a covalent bond, —C(O)—, —C(O)O—, —OC(O)—, —O—, or —OC(O)O—;
R¹ is

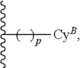

or an optionally substituted saturated or unsaturated, straight or branched C₁-C₂₀ hydrocarbon chain where 1-3 methylene units are optionally and independently replaced with —O— or —NR—;
Cy^{B} is an optionally substituted ring selected from 3- to 12-membered saturated or partially unsaturated carbocyclyl, 1-adamantyl, 2-adamantyl,

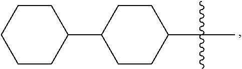 [SMILES: *C1CCC(C2CCCCC2)CC1]

sterolyl, and phenyl;
p is 0, 1, 2, or 3;
X¹ is a covalent bond, —O—, or —NR—;
X² is a covalent bond or an optionally substituted, bivalent saturated or unsaturated, straight or branched, C₁-C₁₂ hydrocarbon chain, where 1-3 methylene units are optionally and independently replaced with —O—, —NR—, or —Cy^{c}—;
Cy^{C} is an optionally substituted ring selected from 3- to 7-membered saturated or partially unsaturated carbocyclene, phenylene, 3- to 7-membered saturated or partially unsaturated heterocyclene having 1-3 heteroatoms independently selected from nitrogen, oxygen, and sulfur, and 5- to 6-membered heteroarylene having 1-3 heteroatoms independently selected from nitrogen, oxygen, and sulfur;
X³ is hydrogen or an optionally substituted ring selected from 3- to 7-membered saturated or partially unsaturated carbocyclyl, phenyl, 3- to 7-membered saturated or partially unsaturated heterocyclyl having 1-3 heteroatoms independently selected from nitrogen, oxygen, and sulfur, or 5- to 6-membered heteroaryl having 1-3 heteroatoms independently selected from nitrogen, oxygen, and sulfur;
each R is independently hydrogen or an optionally substituted C₁-C₆ aliphatic group;
Z¹ is a covalent bond or —O—;
Z² is an optionally substituted group selected from 4- to 12-membered saturated or partially unsaturated carbocyclyl, phenyl, 1-adamantyl, and 2-adamantyl;
Z³ is hydrogen, or an optionally substituted group selected from C₁-C₁₀ aliphatic, and 4- to 12-membered saturated or partially unsaturated carbocyclyl; and
d is 0, 1, 2, 3, 4, 5, or 6; provided that when L3 is a covalent bond, then R¹ must be

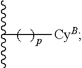

F) an amino lipid of Formula (If):

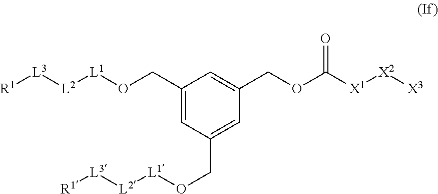

or a pharmaceutically acceptable salt thereof, where:
each L¹ and L^{1′} is independently-C(O)— or —C(O)O—;
each L² and L^{2′} is independently a covalent bond, an optionally substituted bivalent saturated or unsaturated, straight or branched C₁-C₁₂ hydrocarbon chain, or

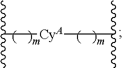

each Cy^{A} is independently an optionally substituted ring selected from phenylene or a 3- to 7-membered saturated or partially unsaturated carbocyclene;
each m is independently 0, 1, or 2;
each L³ and L^{3′} is independently a covalent bond, —C(O)O—, —OC(O)—, —O—, or —OC(O)O—;
each R¹ and R^{1′} is independently an optionally substituted group selected from a saturated or unsaturated, straight or branched C₁-C₂₀ hydrocarbon chain, where 1-3 methylene units are optionally and independently replaced with —O— or —NR—, a 3- to 12-membered saturated or partially unsaturated carbocyclic ring, 1-adamantyl, 2-adamantyl, sterolyl, phenyl, and

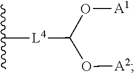

each L⁴ is independently a bivalent saturated or unsaturated, straight or branched C₁-C₆ hydrocarbon chain;
   each A¹ and A² is independently an optionally substituted C₁-C₂₀ aliphatic or —L⁵-R⁵;
      or A¹ and A², together with their intervening atoms, may form an optionally substituted ring:

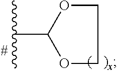

where
   x is selected from 1 or 2; and
   #represents the point of attachment to L⁴;
each L⁵ is independently a bivalent saturated or unsaturated, straight or branched C₁-C₂₀ hydrocarbon chain, where 1-3 methylene units are optionally and independently replaced with —O— or —NR—;
each R⁵ is independently an optionally substituted group selected from a 5- to 10-membered aryl ring and a 3- to 8-membered carbocyclic ring;
X¹ is a covalent bond, —O—, or —NR—;
   X² is a covalent bond or an optionally substituted, bivalent saturated or unsaturated, straight or branched, C₁-C₁₂ hydrocarbon chain, where 1-3 methylene units are optionally and independently replaced with —O— or —NR—; X³ is hydrogen or —Cy^{B};
   Cy^{B} is an optionally substituted ring selected from 3- to 7-membered saturated or partially unsaturated carbocyclyl, phenyl, 3- to 7-membered saturated or partially unsaturated heterocyclyl having 1-3 heteroatoms independently selected from nitrogen, oxygen, and sulfur, and 5- to 6-membered heteroaryl having 1-3 heteroatoms independently selected from nitrogen, oxygen, and sulfur; and
each R is independently hydrogen or an optionally substituted C₁-C₆ aliphatic group;
   provided that when X³ is hydrogen, at least one of R¹ or R^{1′} is

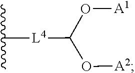

or
G) an amino lipid of Formula (Ig):

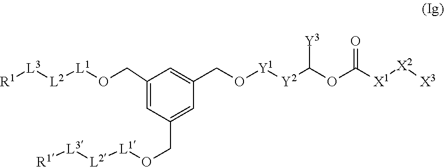

or a pharmaceutically acceptable salt thereof, where:
each of L¹ and L^{1′} is independently a covalent bond, —C(O)—, or —OC(O)—;
each of L² and L^{2′} is independently a covalent bond, an optionally substituted bivalent saturated or unsaturated, straight or branched C₁-C₁₂ hydrocarbon chain, or

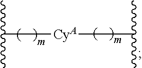

each Cy^{A} is independently an optionally substituted ring selected from phenylene or 3- to 7-membered saturated or partially unsaturated carbocyclene;
each m is independently 0, 1, or 2;
each of L³ and L^{3′} is independently a covalent bond, —O—, —C(O)O—, —OC(O)—, or —OC(O)O—;
each of R¹ and R^{1′} is independently an optionally substituted group selected from saturated or unsaturated, straight or branched C₁-C₂₀ hydrocarbon chain where 1-3 methylene units are optionally and independently replaced with —O— or —NR—, a 3- to 7-membered saturated or partially unsaturated carbocyclic ring, 1-adamantyl, 2-adamantyl, sterolyl, phenyl, or

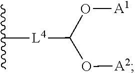

each L⁴ is independently a bivalent saturated or unsaturated, straight or branched C₁-C₂₀ hydrocarbon chain;
each A¹ and A² is independently an optionally substituted C₁-C₂₀ aliphatic or —L⁵—R⁵,
or A¹ and A², together with their intervening atoms, may form an optionally substituted ring:

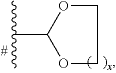

where
   x is selected from 1 or 2; and
   #represents the point of attachment to L⁴;
each L⁵ is independently a bivalent saturated or unsaturated, straight or branched C₁-C₂₀ hydrocarbon chain, where 1-3 methylene units are optionally and independently replaced with —O— or —NR—;
each R⁵ is independently an optionally substituted group selected from a 6- to 10-membered aryl ring or a 3- to 8-membered carbocyclic ring;
Y¹ is a covalent bond, —C(O)—, or —C(O)O—;
Y² is a bivalent saturated or unsaturated, straight or branched C₁-C₆ hydrocarbon chain, where 1-2 methylene units are optionally and independently replaced with cyclopropylene, —O—, or —NR—;
Y³ is an optionally substituted group selected from saturated or unsaturated, straight or branched C₁-C₁₄ hydrocarbon chain, where 1-3 methylene units are optionally and independently replaced with —O— or —NR—, a 3- to 7-membered saturated or partially unsaturated carbocyclic ring, 1-adamantyl, 2-adamantyl, or phenyl;
X¹ is a covalent bond, —O—, or —NR—;
X² is an optionally substituted bivalent saturated or unsaturated, straight or branched C₁-C₁₂ hydrocarbon chain, where 1-3 methylene units are optionally and independently replaced with —O—, —NR—, or —Cy^{B}—;
each Cy^{B} is independently an optionally substituted ring selected from 3- to 7-membered saturated or partially unsaturated carbocyclene, phenylene, 3- to 7-membered heterocyclene having 1-3 heteroatoms independently selected from nitrogen, oxygen, and sulfur, or 5- to 6-membered heteroarylene having 1-3 heteroatoms independently selected from nitrogen, oxygen, and sulfur;
X³ is hydrogen or an optionally substituted ring selected from 3- to 7-membered saturated or partially unsaturated carbocyclyl, phenyl, 3- to 7-membered heterocyclyl having 1-3 heteroatoms independently selected from nitrogen, oxygen, and sulfur, or 5- to 6-membered heteroaryl having 1-3 heteroatoms independently selected from nitrogen, oxygen, and sulfur; and
each R is independently hydrogen or an optionally substituted C₁-C₆ aliphatic group

In another aspect, the disclosure features a pharmaceutical composition containing the LNP of any one of any aspect of the disclosure, or embodiments thereof.

In another aspect, the disclosure features a method of treating a disease or disorder. The method involves administering to a subject in need thereof the pharmaceutical composition of any aspect of the disclosure, or embodiments thereof.

In any aspect of the disclosure, or embodiments thereof, the LNP further contains an amino lipid of Formula A′:

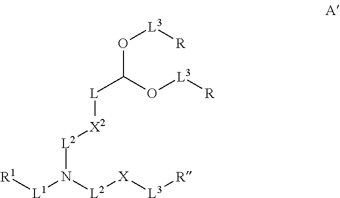

or its N-oxide, or a pharmaceutically acceptable salt thereof, where
L¹ is absent, C₁-6 alkylenyl, or C₂-6 heteroalkylenyl;
each L² is independently optionally substituted C₂₋₁₅ alkylenyl, or optionally substituted C₃₋₁₅ heteroalkylenyl;
L is C₁-10 alkylenyl, or C₂-10 heteroalkylenyl;
X² is —OC(O)—, —C(O)O—, or —OC(O)O—;
X is absent, —OC(O)—, —C(O)O—, or —OC(O)O—;
R″ is hydrogen,

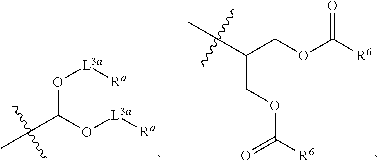

or an optionally substituted group selected from C₆-20 aliphatic, 3- to 12-membered cycloaliphatic, 7- to 12-membered bridged bicyclic containing 0-4 heteroatoms independently selected from nitrogen, oxygen, or sulfur, 1-adamantyl, 2-adamantyl, sterolyl, and phenyl;
each of R and R^{a} is independently hydrogen, or an optionally substituted group selected from C₆-20 aliphatic, 3- to 12-membered cycloaliphatic, 7- to 12-membered bridged bicyclic containing 0-4 heteroatoms independently selected from nitrogen, oxygen, or sulfur, 1-adamantyl, 2-adamantyl, sterolyl, and phenyl
each of L³ and L^{3a} is independently absent, optionally substituted C₁₋₁₀ alkylenyl, or optionally substituted C₂₋₁₀ heteroalkylenyl;
R¹ is hydrogen, optionally substituted phenyl, optionally substituted 3- to 7-membered cycloaliphatic, optionally substituted 3- to 7-membered heterocyclyl containing 1-3 heteroatoms independently selected from nitrogen, oxygen, and sulfur, optionally substituted 5- to 6-membered monocyclic heteroaryl containing 1-4 heteroatoms independently selected from nitrogen, oxygen, and sulfur, optionally substituted 8- to 10-membered bicyclic heteroaryl containing 1-4 heteroatoms independently selected from nitrogen, oxygen, and sulfur, —OR², —C(O)OR², —C(O) SR², —OC(O)R², —OC(O)OR², —CN, —N(R²)₂, —C(O)N(R²)₂, —S(O)₂N(R²)₂, —NR²C(O)R², —OC(O)N(R²)₂, —N(R²)C(O)OR², —NR²S(O)₂R², —NR²C(O)N(R²)₂, —NR²C(S)N(R²)₂, —NR²C(NR²)N(R²)₂, —NR²C(CHR²)N(R²)₂, —N(OR²)C(O)R², —N(OR²) S(O)₂R², —N(OR²)C(O)OR², —N(OR²)C(O)N(R²)₂, —N(OR²)C(S)N(R²)₂, —N(OR²)C(NR²)N(R²)₂, —N(OR²)C(CHR²)N(R²)₂, —C(NR²)N(R²)₂, —C(NR²)R², —C(O)N(R²)OR², —C(R²)N(R²)₂C(O)OR², —CR² (R³)₂, —OP(O)(OR²)₂, or —P(O)(OR²)₂; or
R¹ is

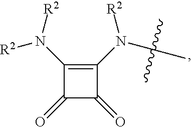

or a ring selected from 3- to 7-membered cycloaliphatic and 3- to 7-membered heterocyclyl containing 1-3 heteroatoms independently selected from nitrogen, oxygen, and sulfur, where the cycloaliphatic or heterocyclyl ring is optionally substituted with 1-4 R² or R³ groups;
each R² is independently hydrogen, oxo, —CN, —NO₂, —OR⁴, —S(O)₂R⁴, —S(O)₂N(R⁴)₂, —(CH₂) n-R⁴, or an optionally substituted group selected from C₁-6 aliphatic, phenyl, 3- to 7-membered cycloaliphatic, 5- to 6-membered monocyclic heteroaryl containing 1-4 heteroatoms independently selected from nitrogen, oxygen, and sulfur, and 3- to 7-membered heterocyclyl containing 1-3 heteroatoms independently selected from nitrogen, oxygen, and sulfur; or
   two occurrences of R², taken together with the atom(s) to which they are attached, form optionally substituted 4- to 7-membered heterocyclyl containing 0-1 additional heteroatom selected from nitrogen, oxygen, and sulfur;
each R³ is independently —(CH₂)ₙ—R⁴; or
   two occurrences of R³, taken together with the atom(s) to which they are attached, form optionally substituted 5- to 6-membered heterocyclyl containing 0-1 additional heteroatom selected from nitrogen, oxygen, and sulfur;
each R⁴ is independently hydrogen, —OR⁵, —N(R⁵)₂, —OC(O)R⁵, —OC(O)OR⁵, —CN, —C(O)N(R⁵)₂,
   —NR⁵C(O)R⁵, —OC(O)N(R⁵)₂, —N(R⁵)C(O)OR⁵, —NR⁵S(O)₂R⁵, —NR⁵C(O)N(R⁵)₂,
   —NR⁵C(S)N(R⁵)₂, —NR⁵C(NR⁵)N(R⁵)₂, or

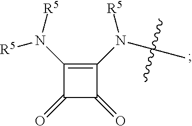

each R⁵ is independently hydrogen, or optionally substituted C₁₋₆ aliphatic; or
   two occurrences of R⁵, taken together with the atom(s) to which they are attached, form optionally substituted 4- to 7-membered heterocyclyl containing 0-1 additional heteroatom selected from nitrogen, oxygen, and sulfur;
each R⁶ is independently C₄-12 aliphatic; and
each n is independently 0 to 4.

In any aspect of the disclosure, or embodiments thereof, the LNP further contains an amino lipid of Formula I:

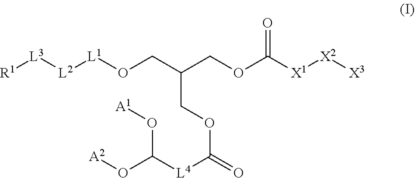

or a pharmaceutically acceptable salt thereof, where:
L¹ is a covalent bond, —C(O)—, or —OC(O)—;
L² is a covalent bond, an optionally substituted bivalent saturated or unsaturated, straight or branched C₁-C₁₂ hydrocarbon chain, or

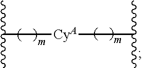

Cy^{A} is an optionally substituted ring selected from phenylene and 3- to 7-membered saturated or partially unsaturated carbocyclene;
each m is independently 0, 1, or 2;
L³ is a covalent bond, —C(O)—, —C(O)O—, —OC(O)—, —O—, or —OC(O)O—;
R¹ is

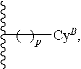

an optionally substituted saturated or unsaturated, straight or branched C₁-C₂₀ hydrocarbon chain where 1-3 methylene units are optionally and independently replaced with —O— or —NR—, or

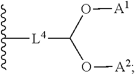

Cy^{B} is an optionally substituted ring selected from 3- to 12-membered saturated or partially unsaturated carbocyclyl, 1-adamantyl, 2-adamantyl,

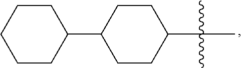 [SMILES: *C1CCC(C2CCCCC2)CC1]

sterolyl, and phenyl;
p is 0, 1, 2, or 3;
each L⁴ is independently a bivalent saturated or unsaturated, straight or branched C₁-C₆ hydrocarbon chain;
each A¹ and A² is independently an optionally substituted C₁-C₂₀ aliphatic or —L⁵—R⁵;
or A¹ and A², together with their intervening atoms, may form an optionally substituted ring:

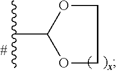

where
   x is selected from 1 or 2; and
   #represents the point of attachment to L⁴;
each L⁵ is independently a bivalent saturated or unsaturated, straight or branched C₁-C₂₀ hydrocarbon chain, where 1-3 methylene units are optionally and independently replaced with —O— or —NR—;
each R⁵ is independently an optionally substituted group selected from a 5- to 10-membered aryl ring or a 3- to 8-membered carbocyclic ring;
X¹ is a covalent bond, —O—, or —NR—;
X² is a covalent bond or an optionally substituted, bivalent saturated or unsaturated, straight or branched, C₁-C₁₂ hydrocarbon chain, where 1-3 methylene units are optionally and independently replaced with —O—, —NR—, or —Cy^{C}—;
Cy^{C} is an optionally substituted ring selected from 3- to 7-membered saturated or partially unsaturated carbocyclene, phenylene, 3- to 7-membered saturated or partially unsaturated heterocyclene having 1-3 heteroatoms independently selected from nitrogen, oxygen, and sulfur, and 5- to 6-membered heteroarylene having 1-3 heteroatoms independently selected from nitrogen, oxygen, and sulfur;
X³ is hydrogen or an optionally substituted ring selected from 3- to 7-membered saturated or partially unsaturated carbocyclyl, phenyl, 3- to 7-membered saturated or partially unsaturated heterocyclyl having 1-3 heteroatoms independently selected from nitrogen, oxygen, and sulfur, or 5- to 6-membered heteroaryl having 1-3 heteroatoms independently selected from nitrogen, oxygen, and sulfur; and
each R is independently hydrogen or an optionally substituted C₁-C₆ aliphatic group; provided that when L³ is a covalent bond, then R¹ must be

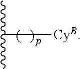

In any aspect of the disclosure, or embodiments thereof, the LNP contains an N: P ratio of between about 1:40 to about 1:1.

In any aspect of the disclosure, or embodiments thereof, the LNP contains an N: P ratio of about 1:6.

In any aspect of the disclosure, or embodiments thereof, the guide polynucleotide contains a scaffold sequence selected from the following:
GUUUUAGAGCUAGAAAUAGCAAGUUAAAAUAAGGCUAGUCCGUUAUCAACUUGAAAAAGUGG CACCGAGUCGGUGCmU*mU*mU*U (SEQ ID NO: 317);
mGUUUUAGmAmGmCmUmAGmAmAmAmUmAmGmCmAmAGUUmAAmAAmUAmAmGmGmCmUmAG UmCmCGUUAmUmCAAmCmUmUGmAmAmAmAmAmGmUmGGmCmAmCmCmGmAmGmUmCmGmGm UmGmCmU*mU*mU*mU (SEQ ID NO: 317), and
mG*U*U*U*U*A*G*mA*mG*mC*mU*mA*Gm*Am*Am*Am*Um*Am*Gm*Cm*Am*A*G*U *Um*A*A*mA*A*mU*A*mA*mG*mG*mC*mU*mA*G*U*mC*mC*G*U*U*A*mU*mC*A* A*mC*mU*mU*G*mA*mA*mA*mA*mA*mG*mU*mG*G*mC*mA*mC*mC*mG*mA*mG*mU *mC*mG*mG*mU*mG*mC*mU*mU*mU*mU (SEQ ID NO: 317), where A is adenosine, C is cytidine, G is guanosine, U is uridine, mA is 2′-O-methyladenosine, mC is 2′-O-methylcytidine, mG is 2′-O-methylguanosine, mU is 2′-O-methyluridine, and “*” indicates a phosphorothioate (PS) backbone linkage. In any aspect of the disclosure, or embodiments thereof, the guide polynucleotide contains 2-5 contiguous 2′-O-methylated nucleobases at the 3′ end and at the 5′ end. In any aspect of the disclosure, or embodiments thereof, the guide polynucleotide contains 2-5 contiguous nucleobases at the 3′ end and at the 5′ end that contain phosphorothioate internucleotide linkages.

In any aspect of the disclosure, or embodiments thereof, the LNP further contains a polynucleotide encoding a base editor containing a nucleic acid programmable DNA binding protein (napDNAbp) and a deaminase domain. In any aspect of the disclosure, or embodiments thereof, the LNP further contains a polynucleotide encoding a nuclease active nucleic acid programmable DNA binding protein (napDNAbp).

In any aspect of the disclosure, or embodiments thereof, the disease or disorder is hereditary transthyretin amyloidosis, cardiomyopathy, polyneuropathy or senile cardiac amyloidosis.

In any aspect of the disclosure, or embodiments thereof, the pharmaceutical composition is administered by a route selected from intravenous, intradermal, transdermal, intranasal, intramuscular, subcutaneous, transmucosal or oral.

In any aspect of the disclosure, or embodiments thereof, the LNP is delivered to liver.

In any aspect provided herein, or embodiments thereof, the method is not a process for modifying the germline genetic identity of human beings.

In any aspect of the disclosure, or embodiments thereof, the amino lipid is a compound of Formula III-a-i:

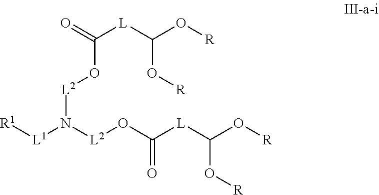

or its N-oxide, or a pharmaceutically acceptable salt thereof, where each of R, R¹, L, L¹, and L² is as defined for Formula A′ of the disclosure.

In any aspect of the disclosure, or embodiments thereof, the amino lipid is a compound of the formula BLP8-4:

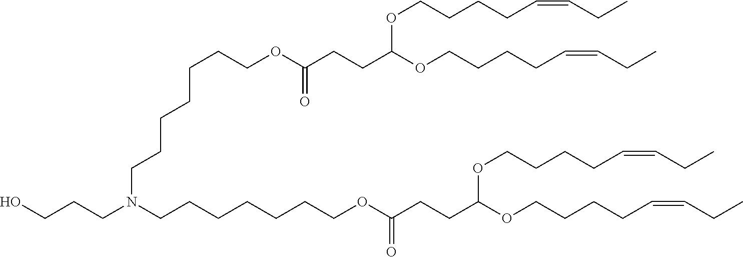 [SMILES: CC/C=C\CCCCOC(CCC(=O)OCCCCCCCN(CCCO)CCCCCCCOC(=O)CCC(OCCCC/C=C\CC)OCCCC/C=C\CC)OCCCC/C=C\CC]

or pharmaceutically acceptable salt thereof.

In any aspect of the disclosure, or embodiments thereof, the amino lipid is a compound of Formula (VIA):

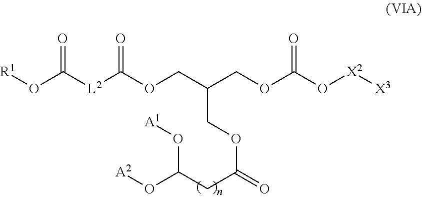

or a pharmaceutically acceptable salt thereof, where n is 1, 2, 3 or 4, and L², R¹, A¹, A², X², and X³ are as defined for Formula I of the disclosure.

In any aspect of the disclosure, or embodiments thereof, the amino lipid is a compound of the formula BLP4-71:

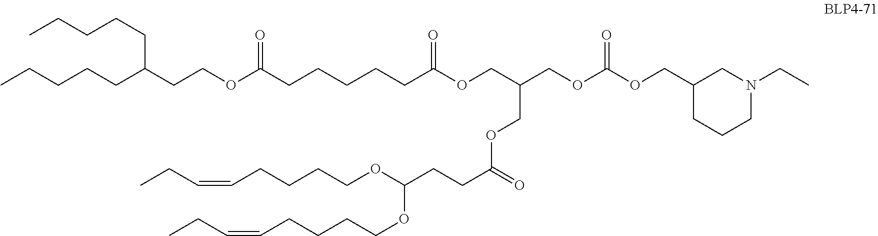 [SMILES: CC/C=C\CCCCOC(CCC(=O)OCC(COC(=O)CCCCCC(=O)OCCC(CCCCC)CCCCC)COC(=O)OCC1CCCN(CC)C1)OCCCC/C=C\CC]

or a pharmaceutically acceptable salt thereof.

### Definitions

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “adenine” or “9H-Purin-6-amine” is meant a purine nucleobase with the molecular formula C₅H₅N₅, having the structure

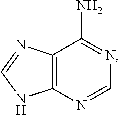

and corresponding to CAS No. 73-24-5.

By “adenosine” or “4-Amino-1-[(2R,3R,4S,5R)-3,4-dihydroxy-5-(hydroxymethyl)oxolan-2-yl]pyrimidin-2 (1H)-one” is meant an adenine molecule attached to a ribose sugar via a glycosidic bond, having the structure

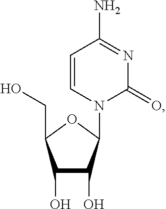

and corresponding to CAS No. 65-46-3. Its molecular formula is C₁₀H₁₃N₅O₄.

By “adenosine deaminase” or “adenine deaminase” is meant a polypeptide or fragment thereof capable of catalyzing the hydrolytic deamination of adenine or adenosine. In some embodiments, the deaminase or deaminase domain is an adenosine deaminase catalyzing the hydrolytic deamination of adenosine to inosine or deoxy adenosine to deoxyinosine. In some embodiments, the adenosine deaminase catalyzes the hydrolytic deamination of adenine or adenosine in deoxyribonucleic acid (DNA). The adenosine deaminases (e.g., engineered adenosine deaminases, evolved adenosine deaminases) provided herein may be from any organism (e.g., eukaryotic, prokaryotic), including but not limited to algae, bacteria, fungi, plants, invertebrates (e.g., insects), and vertebrates (e.g., amphibians, mammals). In some embodiments, the adenosine deaminase is an adenosine deaminase variant with one or more alterations and is capable of deaminating both adenine and cytosine in a target polynucleotide (e.g., DNA, RNA) and may be referred to as a “dual deaminase”. Non-limiting examples of dual deaminases include those described in PCT/US22/22050. In some embodiments, the target polynucleotide is single or double stranded. In some embodiments, the adenosine deaminase variant is capable of deaminating both adenine and cytosine in DNA. In some embodiments, the adenosine deaminase variant is capable of deaminating both adenine and cytosine in single-stranded DNA. In some embodiments, the adenosine deaminase variant is capable of deaminating both adenine and cytosine in RNA. In embodiments, the adenosine deaminase variant is selected from those described in PCT/US2020/018192, PCT/US2020/049975, PCT/US2017/045381, and PCT/US2020/028568, the full contents of which are each incorporated herein by reference in their entireties for all purposes.

By “adenosine deaminase activity” is meant catalyzing the deamination of adenine or adenosine to guanine in a polynucleotide. In some embodiments, an adenosine deaminase variant as provided herein maintains adenosine deaminase activity (e.g., at least about 30%, 40%, 50%, 60%, 70%, 80%, 90% or more of the activity of a reference adenosine deaminase (e.g., TadA*8.20 or TadA*8.19)).

By “Adenosine Base Editor (ABE)” is meant a base editor comprising an adenosine deaminase.

By “Adenosine Base Editor (ABE) polynucleotide” is meant a polynucleotide encoding an ABE.

By “Adenosine Base Editor 8 (ABE8) polypeptide” or “ABE8” is meant a base editor as defined herein comprising an adenosine deaminase or adenosine deaminase variant comprising one or more of the alterations listed in Table 5B, one of the combinations of alterations listed in Table 5B, or an alteration at one or more of the amino acid positions listed in Table 5B, such alterations are relative to the following reference sequence: MSEVEFSHEYWMRHALTLAKRARDEREVPVGAVLVLNNRVIGEGWNRAIGLHDPTAHAEIMA LRQGGLVMQNYRLIDATLYVTFEPCVMCAGAMIHSRIGRVVFGVRNAKTGAAGSLMDVLHYP GMNHRVEITEGILADECAALLCYFFRMPRQVFNAQKKAQSSTD (SEQ ID NO: 1), or a corresponding position in another adenosine deaminase. In embodiments, ABE8 comprises alterations at amino acids 82 and/or 166 of SEQ ID NO: 1. In some embodiments, ABE8 comprises further alterations, as described herein, relative to the reference sequence.

By “Adenosine Base Editor 8 (ABE8) polynucleotide” is meant a polynucleotide encoding an ABE8 polypeptide.

By “Adenosine Base Editor 8.8 (ABE8.8)” or “ABE8.8” is meant a base editor as defined herein comprising an adenosine deaminase variant comprising the alterations Y¹²³H, Y¹⁴⁷R, and Q154R relative to the following reference sequence:

(SEQ ID NO: 1
MSEVEFSHEYWMRHALTLAKRARDEREVPVGAVLVLNNRVIGEGWNRAIG
LHDPTAHAEIMALRQGGLVMQNYRLIDATLYVTFEPCVMCAGAMIHSRIG
RVVFGVRNAKTGAAGSLMDVLHYPGMNHRVEITEGILADECAALLCYFFR
MPRQVFNAQKKAQSSTD;

TadA*7.10), or a corresponding position in another adenosine deaminase. In some embodiments, ABE8.8 comprises further alterations (e.g., 1, 2, 3, 4, 5, 6, 7, 8, 9, 10, 11, 13, 14, or 15 alterations) relative to the reference sequence, or a corresponding position in another adenosine deaminase.

By “Adenosine Base Editor 8.8 (ABE8.8) polynucleotide” is meant a polynucleotide encoding an ABE8.8 polypeptide.

By “Adenosine Base Editor 8.13 (ABE8.13) polypeptide” or “ABE8.13” is meant a base editor as defined herein comprising an adenosine deaminase variant comprising the alterations I76Y, Y¹²³H, Y¹⁴⁷R, and Q154R relative to the following reference sequence:

(SEQ ID NO: 1
MSEVEFSHEYWMRHALTLAKRARDEREVPVGAVLVLNNRVIGEGWNRAIG
LHDPTAHAEIMALRQGGLVMQNYRLIDATLYVTFEPCVMCAGAMIHSRI
GRVVFGVRNAKTGAAGSLMDVLHYPGMNHRVEITEGILADECAALLCYFF
RMPRQVFNAQKKAQSSTD;

TadA*7.10). In some embodiments, ABE8.13 comprises further alterations (e.g., 1, 2, 3, 4, 5, 6, 7, 8, 9, 10, 11, 13, 14, or 15 alterations) relative to the reference sequence.

By “Adenosine Base Editor 8.13 (ABE8.13) polynucleotide” is meant a polynucleotide encoding an ABE8.13 polypeptide.

“Administering” is referred to herein as providing one or more compositions described herein to a patient or a subject. By way of example and without limitation, composition administration (e.g., injection) can be performed by intravenous (i.v.) injection, sub-cutaneous (s.c.) injection, intradermal (i.d.) injection, intraperitoneal (i.p.) injection, or intramuscular (i.m.) injection. One or more such routes can be employed. Parenteral administration can be, for example, by bolus injection or by gradual perfusion over time. In some embodiments, parenteral administration includes infusing or injecting intravascularly, intravenously, intramuscularly, intraarterially, intrathecally, intratumorally, intradermally, intraperitoneally, transtracheally, subcutaneously, subcuticularly, intraarticularly, subcapsularly, subarachnoidly and intrasternally. Alternatively, or concurrently, administration can be by the oral route.

By “agent” is meant any small molecule chemical compound, antibody, nucleic acid molecule, or polypeptide, or fragments thereof.

By “alteration” is meant a change in the level, structure, or activity of an analyte, gene or polypeptide as detected by standard art known methods such as those described herein. As used herein, an alteration includes a change (e.g., increase or reduction) in expression levels. In embodiments, the increase or reduction in expression levels is by 10%, 25%, 40%, 50% or greater. In some embodiments, an alteration includes an insertion, deletion, or substitution of a nucleobase or amino acid (by, e.g., genetic engineering).

By “ameliorate” is meant reduce, suppress, attenuate, diminish, arrest, or stabilize the development or progression of a disease.

By “amyloidosis” is meant a disease associated with buildup of amyloid in a tissue of a subject. In embodiments, amyloidosis affects the nervous system (e.g., central nervous system), heart, or liver.

By “analog” is meant a molecule that is not identical but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “base editor (BE),” or “nucleobase editor polypeptide (NBE)” is meant an agent that binds a polynucleotide and has nucleobase modifying activity. In various embodiments, the base editor comprises a nucleobase modifying polypeptide (e.g., a deaminase) and a polynucleotide programmable nucleotide binding domain (e.g., Cas9 or Cpf1). Representative nucleic acid and protein sequences of base editors include those sequences having about or at least about 85% sequence identity to any base editor sequence provided in the sequence listing, such as those corresponding to SEQ ID NOs: 2-11.

By “BE4 cytidine deaminase (BE4) polypeptide,” is meant a base editor comprising a nucleic acid programmable DNA binding protein (napDNAbp) domain, a cytidine deaminase domain, and two uracil glycosylase inhibitor domains (UGIs). In embodiments, the napDNAbp is a Cas9n (D10A) polypeptide. Non-limiting examples of cytidine deaminase domains include rAPOBEC, ppAPOBEC, RrA3F, AmAPOBEC1, and SsAPOBEC3B. In an embodiment, a BE4 polypeptide shares at least 85% sequence identity to the following reference sequence:
MSSETGPVAVDPTLRRRIEPHEFEVFFDPRELRKETCLLYEINWGGRHSIWRHTSQNTNKHV EVNFIEKFTTERYFCPNTRCSITWFLSWSPCGECSRAITEFLSRYPHVTLFIYIARLYHHAD PRNRQGLRDLISSGVTI QIMTEQESGYCWRNFVNYSPSNEAHWPRYPHLWVRLYVLELYCII LGLPPCLNILRRKQPQLTFFTIALQSCHYQRLPPHILWATGLK (SEQ ID NO: 21; BE4 cytidine deaminase domain). In some embodiments, BE4 comprises further alterations (e.g., 1, 2, 3, 4, 5, 6, 7, 8, 9, 10, 11, 13, 14, or 15 alterations) relative to the reference sequence.

By “BE4 cytidine deaminase (BE4) polynucleotide,” is meant a polynucleotide encoding a BE4 polypeptide.

By “base editing activity” is meant acting to chemically alter a base within a polynucleotide. In one embodiment, a first base is converted to a second base. In one embodiment, the base editing activity is cytidine deaminase activity, e.g., converting target C·G to T·A. In another embodiment, the base editing activity is adenosine or adenine deaminase activity, e.g., converting A·T to G·C.

The term “base editor system” refers to an intermolecular complex for editing a nucleobase of a target nucleotide sequence. In various embodiments, the base editor (BE) system comprises (1) a polynucleotide programmable nucleotide binding domain, a deaminase domain (e.g., cytidine deaminase or adenosine deaminase) for deaminating nucleobases in the target nucleotide sequence; and (2) one or more guide polynucleotides (e.g., guide RNA) in conjunction with the polynucleotide programmable nucleotide binding domain. In various embodiments, the base editor (BE) system comprises a nucleobase editor domain selected from an adenosine deaminase or a cytidine deaminase, and a domain having nucleic acid sequence specific binding activity. In some embodiments, the base editor system comprises (1) a base editor (BE) comprising a polynucleotide programmable DNA binding domain and a deaminase domain for deaminating one or more nucleobases in a target nucleotide sequence; and (2) one or more guide RNAs in conjunction with the polynucleotide programmable DNA binding domain. In some embodiments, the polynucleotide programmable nucleotide binding domain is a polynucleotide programmable DNA binding domain. In some embodiments, the base editor is a cytidine base editor (CBE). In some embodiments, the base editor is an adenine or adenosine base editor (ABE). In some embodiments, the base editor is an adenine or adenosine base editor (ABE) or a cytidine or cytosine base editor (CBE). In some embodiments, the base editor system (e.g., a base editor system comprising a cytidine deaminase) comprises a uracil glycosylase inhibitor or other agent or peptide (e.g., a uracil stabilizing protein such as provided in WO2022015969, the disclosure of which is incorporated herein by reference in its entirety for all purposes) that inhibits the inosine base excision repair system.

The term “Cas9” or “Cas9 domain” refers to an RNA guided nuclease comprising a Cas9 protein, or a fragment thereof (e.g., a protein comprising an active, inactive, or partially active DNA cleavage domain of Cas9, and/or the gRNA binding domain of Cas9). A Cas9 nuclease is also referred to sometimes as a casnl nuclease or a CRISPR (clustered regularly interspaced short palindromic repeat) associated nuclease.

By “bhCas12b v4 polypeptide” or “bhCas12b v4” is meant an endonuclease variant comprising a sequence with at least about 85% sequence identity to the following reference sequence and having endonuclease activity:
MAPKKKRKVGIHGVPAAATRSFILKIEPNEEVKKGLWKTHEVLNHGIAYYMNILKLIRQEAI YEHHEQDPKNPKKVSKAEIQAELWDFVLKMQKCNSFTHEVDKDEVFNILRELYEELVPSSVE KKGEANQLSNKFLYPLVDPNSQSGKGTASSGRKPRWYNLKIAGDPSWEEEKKKWEEDKKKDP LAKILGKLAEYGLIPLFIPYTDSNEPIVKEIKWMEKSRNQSVRRLDKDMFIQALERFLSWES WNLKVKEEYEKVEKEYKTLEERIKEDIQALKALEQYEKERQEQLLRDTLNTNEYRLSKRGLR GWREIIQKWLKMDENEPSEKYLEVFKDYQRKHPREAGDYSVYEFLSKKENHFIWRNHPEYPY LYATFCEIDKKKKDAKQQATFTLADPINHPLWVRFEERSGSNLNKYRILTEQLHTEKLKKKL TVQLDRLIYPTESGGWEEKGKVDIVLLPSRQFYNQIFLDIEEKGKHAFTYKDESIKFPLKGT LGGARVQFDRDHLRRYPHKVESGNVGRIYFNMTVNIEPTESPVSKSLKIHRDDFPKVVNFKP KELTEWIKDSKGKKLKSGIESLEIGLRVMSIDLGQRQAAAASIFEVVDQKPDIEGKLFFPIK GTELYAVHRASFNIKLPGETLVKSREVLRKAREDNLKLMNQKLNFLRNVLHFQQFEDITERE KRVTKWISRQENSDVPLVYQDELIQIRELMYKPYKDWVAFLKQLHKRLEVEIGKEVKHWRKS LSDGRKGLYGISLKNIDEIDRTRKFLLRWSLRPTEPGEVRRLEPGORFAIDQLNHLNALKED RLKKMANTIIMHALGYCYDVRKKKWQAKNPACQIILFEDLSNYNPYGERSRFENSRLMKWSR REIPRQVALQGEIYGLQVGEVGAQFSSRFHAKTGSPGIRCRVVTKEKLQDNRFFKNLQREGR LTLDKIAVLKEGDLYPDKGGEKFISLSKDRKCVTTHADINAAQNLQKRFWTRTHGFYKVYCK AYQVDGQTVYIPESKDQKQKIIEEFGEGYFILKDGVYEWVNAGKLKIKKGSSKQSSSELVDS DILKDSFDLASELKGEKLMLYRDPSGNVFPSDKWMAAGVFFGKLERILISKLTNQYSISTIE DDSSKQSMSGGSKRTADGSEFESPKKKRKVE (SEQ ID NO: 463). In some embodiments, bhCAS12b v4 comprises further alterations (e.g., 1, 2, 3, 4, 5, 6, 7, 8, 9, 10, 11, 13, 14, or 15 alterations) relative to the reference sequence.

By “bhCas12b v4 polynucleotide” is meant a polynucleotide encoding a bhCas12b v4.

The term “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz, G. E. and Schirmer, R. H., Principles of Protein Structure, Springer-Verlag, New York (1979)). According to such analyses, groups of amino acids can be defined where amino acids within a group exchange preferentially with each other, and therefore resemble each other most in their impact on the overall protein structure (Schulz, G. E. and Schirmer, R. H., supra). Non-limiting examples of conservative mutations include amino acid substitutions of amino acids, for example, lysine for arginine and vice versa such that a positive charge can be maintained; glutamic acid for aspartic acid and vice versa such that a negative charge can be maintained; serine for threonine such that a free-OH can be maintained; and glutamine for asparagine such that a free —NH₂ can be maintained.

The term “coding sequence” or “protein coding sequence” as used interchangeably herein refers to a segment of a polynucleotide that codes for a protein. Coding sequences can also be referred to as open reading frames. The region or sequence is bounded nearer the 5′ end by a start codon and nearer the 3′ end with a stop codon. Stop codons useful with the base editors described herein include the following: TAG, TAA, and TGA.

By “complex” is meant a combination of two or more molecules whose interaction relies on inter-molecular forces. Non-limiting examples of inter-molecular forces include covalent and non-covalent interactions. Non-limiting examples of non-covalent interactions include hydrogen bonding, ionic bonding, halogen bonding, hydrophobic bonding, van der Waals interactions (e.g., dipole-dipole interactions, dipole-induced dipole interactions, and London dispersion forces), and x-effects. In an embodiment, a complex comprises polypeptides, polynucleotides, or a combination of one or more polypeptides and one or more polynucleotides. In one embodiment, a complex comprises one or more polypeptides that associate to form a base editor (e.g., base editor comprising a nucleic acid programmable DNA binding protein, such as Cas9, and a deaminase) and a polynucleotide (e.g., a guide RNA). In an embodiment, the complex is held together by hydrogen bonds. It should be appreciated that one or more components of a base editor (e.g., a deaminase, or a nucleic acid programmable DNA binding protein) may associate covalently or non-covalently. As one example, a base editor may include a deaminase covalently linked to a nucleic acid programmable DNA binding protein (e.g., by a peptide bond). Alternatively, a base editor may include a deaminase and a nucleic acid programmable DNA binding protein that associate noncovalently (e.g., where one or more components of the base editor are supplied in trans and associate directly or via another molecule such as a protein or nucleic acid). In an embodiment, one or more components of the complex are held together by hydrogen bonds.

By “cytosine” or “4-Aminopyrimidin-2 (1H)-one” is meant a purine nucleobase with the molecular formula C₄H₅N₃₀, having the structure

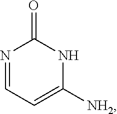

and corresponding to CAS No. 71-30-7.

By “cytidine” is meant a cytosine molecule attached to a ribose sugar via a glycosidic bond, having the structure

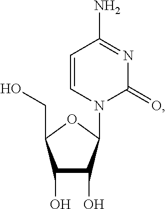

and corresponding to CAS No. 65-46-3. Its molecular formula is C₉H₁₃N₃O₅.

By “Cytidine Base Editor (CBE)” is meant a base editor comprising a cytidine deaminase.

By “Cytidine Base Editor (CBE) polynucleotide” is meant a polynucleotide encoding a CBE.

By “cytidine deaminase” or “cytosine deaminase” is meant a polypeptide or fragment thereof capable of deaminating cytidine or cytosine. In embodiments, the cytidine or cytosine is present in a polynucleotide. In one embodiment, the cytidine deaminase converts cytosine to uracil or 5-methylcytosine to thymine. The terms “cytidine deaminase” and “cytosine deaminase” are used interchangeably throughout the application. Petromyzon marinus cytosine deaminase 1 (PmCDA1) (SEQ ID NO: 13-14), Activation-induced cytidine deaminase (AICDA) (SEQ ID NOs: 15-21), and APOBEC(SEQ ID NOs: 12-61) are exemplary cytidine deaminases. Further exemplary cytidine deaminase (CDA) sequences are provided in the Sequence Listing as SEQ ID NOs: 62-66 and SEQ ID NOs: 67-189. Non-limiting examples of cytidine deaminases include those described in PCT/US20/16288, PCT/US2018/021878, 180802-021804/PCT, PCT/US2018/048969, and PCT/US2016/058344.

By “cytosine deaminase activity” is meant catalyzing the deamination of cytosine or cytidine. In one embodiment, a polypeptide having cytosine deaminase activity converts an amino group to a carbonyl group. In an embodiment, a cytosine deaminase converts cytosine to uracil (i.e., C to U) or 5-methylcytosine to thymine (i.e., 5mC to T). In some embodiments, a cytosine deaminase as provided herein has increased cytosine deaminase activity (e.g., at least 10-fold, 20-fold, 30-fold, 40-fold, 50-fold, 60-fold, 70-fold, 80-fold, 90-fold, 100-fold or more) relative to a reference cytosine deaminase.

The term “deaminase” or “deaminase domain,” as used herein, refers to a protein or fragment thereof that catalyzes a deamination reaction.

“Detect” refers to identifying the presence, absence or amount of the analyte to be detected. In one embodiment, a sequence alteration in a polynucleotide or polypeptide is detected. In another embodiment, the presence of indels is detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an enzyme linked immunosorbent assay (ELISA)), biotin, digoxigenin, or haptens.

By “disease” is meant any condition or disorder that damages or interferes with the normal function of a cell, tissue, or organ. Exemplary diseases include diseases amenable to treatment using the methods and/or compositions of the present disclosure include as non-limiting examples amyloidosis, cardiomyopathy, familial amyloid polyneuropathy (FAP), familial amyloid cardiomyopathy (FAC), familial transthyretin amyloidosis (FTA), senile systemic amyloidosis (SSA), transthyretin amyloidosis, and the like. The disease can be any disease associated with a mutation to a transthyretin (TTR) polynucleotide sequence.

By “dual editing activity” or “dual deaminase activity” is meant having adenosine deaminase and cytidine deaminase activity. In one embodiment, a base editor having dual editing activity has both A→G and C→T activity, wherein the two activities are approximately equal or are within about 10% or 20% of each other. In another embodiment, a dual editor has A→G activity that no more than about 10% or 20% greater than C→T activity. In another embodiment, a dual editor has A→G activity that is no more than about 10% or 20% less than C→T activity. In some embodiments, the adenosine deaminase variant has predominantly cytosine deaminase activity, and little, if any, adenosine deaminase activity. In some embodiments, the adenosine deaminase variant has cytosine deaminase activity, and no significant or no detectable adenosine deaminase activity.

By “effective amount” is meant the amount of an agent (e.g., a base editor, cell) as described herein, that is required to ameliorate the symptoms of a disease relative to an untreated patient or an individual without disease, i.e., a healthy individual, or is the amount of the agent sufficient to elicit a desired biological response. The effective amount of active compound(s) used to practice embodiments of the present disclosure for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. In one embodiment, an effective amount is the amount of a base editor of the disclosure sufficient to introduce an alteration in a gene of interest in a cell (e.g., a cell in vitro or in vivo). In one embodiment, an effective amount is the amount of a base editor required to achieve a therapeutic effect. Such therapeutic effect need not be sufficient to alter a pathogenic gene in all cells of a subject, tissue or organ, but only to alter the pathogenic gene in about 1%, 5%, 10%, 25%, 50%, 75% or more of the cells present in a subject, tissue or organ. In one embodiment, an effective amount is sufficient to ameliorate one or more symptoms of a disease.

The term “exonuclease” refers to a protein or polypeptide capable of removing successive nucleotides from either the 5′ or 3′ end of a polynucleotide.

The term “endonuclease” refers to a protein or polypeptide capable of catalyzing the cleavage of internal regions in a polynucleotide.

By “fragment” is meant a portion of a polypeptide or nucleic acid molecule. This portion contains, at least about 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids. In some embodiments, the fragment is a functional fragment.

The term “gene editing” or “gene modification” and its grammatical equivalents as used herein refers to genetic engineering in which one or more nucleotides are inserted, replaced, or removed from a genome. Gene editing can be performed using a nuclease (e.g., a natural-existing nuclease or an artificially engineered nuclease). Gene modification can include introducing a double stranded break, a non-sense mutation, a frameshift mutation, a splice site alteration, or an inversion in a polynucleotide sequence, e.g., a target polynucleotide sequence. FIG. 1A depicts a crispr Cas9 protein which is an RNA-guided endonuclease that can be used to impart a double-stranded break at a site-specific location in DNA or a gene. Gene modification can also be accomplished using other editors, such as base editors.

By “guide polynucleotide” is meant a polynucleotide or polynucleotide complex which is specific for a target sequence and can form a complex with a polynucleotide programmable nucleotide binding domain protein (e.g., Cas9 or Cpf1). In an embodiment, the guide polynucleotide is a guide RNA (gRNA). gRNAs can exist as a complex of two or more RNAs, or as a single RNA molecule.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “increases” is meant a positive alteration of at least 10%, 25%, 50%, 75%, or 100%, or about 1.5 fold, about 2 fold, about 3-fold, about 4-fold, about 5-fold, about 6-fold, about 7-fold, about 8-fold, about 9-fold, about 10-fold, about 15-fold, about 20-fold, about 25-fold, about 30-fold, about 35-fold, about 40-fold, about 45-fold, about 50-fold, or about 100-fold.

The terms “inhibitor of base repair”, “base repair inhibitor”, “IBR” or their grammatical equivalents refer to a protein that is capable in inhibiting the activity of a nucleic acid repair enzyme, for example a base excision repair enzyme.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this disclosure is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid molecule that is free of the genes which, in the naturally-occurring genome of the organism from which the nucleic acid molecule of the disclosure is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the disclosure that has been separated from components that naturally accompany it. Typically, the polypeptide is isolated when it is at least 60%, by weight, free from the proteins and naturally-occurring organic molecules with which it is naturally associated. In embodiments, the preparation is at least 75%, at least 90%, or at least 99%, by weight, a polypeptide of the disclosure. An isolated polypeptide of the disclosure may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

The term “linker”, as used herein, refers to a molecule that links two moieties. In one embodiment, the term “linker” refers to a covalent linker (e.g., covalent bond) or a non-covalent linker.

By “marker” is meant any protein or polynucleotide having an alteration in expression, level, structure, or activity that is associated with a disease or disorder. In an embodiment, the marker is an accumulation of amyloid protein. In an embodiment, the marker is an alteration (e.g., mutation) in the sequence of a in transthyretin polypeptide and/or a transthyretin polynucleotide.

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4th ed., Cold Spring Harbor Laboratory Press, Cold Spring Harbor, N.Y. (2012)).

The terms “nucleic acid” and “nucleic acid molecule,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g., nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, IRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O (6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′—N-phosphoramidite linkages).

The term “nuclear localization sequence,” “nuclear localization signal,” or “NLS” refers to an amino acid sequence that promotes import of a protein into the cell nucleus. Nuclear localization sequences are known in the art and described, for example, in Plank et al., International PCT application, PCT/EP2000/011690, filed Nov. 23, 2000, published as WO/2001/038547 on May 31, 2001, the contents of which are incorporated herein by reference for their disclosure of exemplary nuclear localization sequences. In other embodiments, the NLS is an optimized NLS described, for example, by Koblan et al., Nature Biotech. 2018 doi: 10.1038/nbt.4172. In some embodiments, an NLS comprises the amino acid sequence KRTADGSEFESPKKKRKV (SEQ ID NO: 190), KRPAATKKAGQAKKKK (SEQ ID NO: 191), KKTELQTTNAENKTKKL (SEQ ID NO: 192), KRGINDRNFWRGENGRKTR (SEQ ID NO: 193), RKSGKIAAIVVKRPRK (SEQ ID NO: 194), PKKKRKV (SEQ ID NO: 195), MDSLLMNRRKFLYQFKNVRWAKGRRETYLC(SEQ ID NO: 196), PKKKRKVEGADKRTADGSEFESPKKKRKV (SEQ ID NO: 328), or RKSGKIAAIVVKRPRKPKKKRKV (SEQ ID NO: 329).

The term “nucleobase,” “nitrogenous base,” or “base,” used interchangeably herein, refers to a nitrogen-containing biological compound that forms a nucleoside, which in turn is a component of a nucleotide. The ability of nucleobases to form base pairs and to stack one upon another leads directly to long-chain helical structures such as ribonucleic acid (RNA) and deoxyribonucleic acid (DNA). Five nucleobases-adenine (A), cytosine (C), guanine (G), thymine (T), and uracil (U)—are called primary or canonical. Adenine and guanine are derived from purine, and cytosine, uracil, and thymine are derived from pyrimidine. DNA and RNA can also contain other (non-primary) bases that are modified. Non-limiting exemplary modified nucleobases can include hypoxanthine, xanthine, 7-methylguanine, 5,6-dihydrouracil, 5-methylcytosine (m5C), and 5-hydromethylcytosine. Hypoxanthine and xanthine can be created through mutagen presence, both of them through deamination (replacement of the amine group with a carbonyl group). Hypoxanthine can be modified from adenine. Xanthine can be modified from guanine. Uracil can result from deamination of cytosine. A “nucleoside” consists of a nucleobase and a five carbon sugar (either ribose or deoxyribose). Examples of a nucleoside include adenosine, guanosine, uridine, cytidine, 5-methyluridine (m5U), deoxyadenosine, deoxyguanosine, thymidine, deoxyuridine, and deoxycytidine. Examples of a nucleoside with a modified nucleobase includes inosine (I), xanthosine (X), 7-methylguanosine (m7G), dihydrouridine (D), 5-methylcytidine (m5C), and pseudouridine ( ) A “nucleotide” consists of a nucleobase, a five carbon sugar (either ribose or deoxyribose), and at least one phosphate group. Non-limiting examples of modified nucleobases and/or chemical modifications that a modified nucleobase may include are the following: pseudo-uridine, 5-Methyl-cytosine, 2′-O-methyl-3′-phosphonoacetate, 2′-O-methyl thioPACE (MSP), 2′-O-methyl-PACE (MP), 2′-fluoro RNA (2′-F-RNA), constrained ethyl (S-cEt), 2′-O-methyl (‘M’), 2′-O-methyl-3′-phosphorothioate (‘MS’), 2′-O-methyl-3′-thiophosphonoacetate (‘MSP’), 5-methoxyuridine, phosphorothioate, and N₁-Methylpseudouridine.

The term “nucleic acid programmable DNA binding protein” or “napDNAbp” may be used interchangeably with “polynucleotide programmable nucleotide binding domain” to refer to a protein that associates with a nucleic acid (e.g., DNA or RNA), such as a guide nucleic acid or guide polynucleotide (e.g., gRNA), that guides the napDNAbp to a specific nucleic acid sequence. In some embodiments, the polynucleotide programmable nucleotide binding domain is a polynucleotide programmable DNA binding domain. In some embodiments, the polynucleotide programmable nucleotide binding domain is a polynucleotide programmable RNA binding domain. In some embodiments, the polynucleotide programmable nucleotide binding domain is a Cas9 protein. A Cas9 protein can associate with a guide RNA that guides the Cas9 protein to a specific DNA sequence that is complementary to the guide RNA. In some embodiments, the napDNAbp is a Cas9 domain, for example a nuclease active Cas9, a Cas9 nickase (nCas9), or a nuclease inactive Cas9 (dCas9). Non-limiting examples of nucleic acid programmable DNA binding proteins include, Cas9 (e.g., dCas9 and nCas9), Cas12a/Cpf1, Cas12b/C₂cl, Cas12c/C₂c3, Cas12d/CasY, Cas12e/CasX, Cas12g, Cas12h, Cas12i, and Cas12j/CasΘ (Cas12j/Casphi). Non-limiting examples of Cas enzymes include Cas1, Cas1B, Cas2, Cas3, Cas4, Cas5, Cas5d, Cas5t, Cas5h, Cas5a, Cas6, Cas7, Cas8, Cas8a, Cas8b, Cas8c, Cas9 (also known as Csn1 or Csx12), Cas10, Cas10d, Cas12a/Cpf1, Cas12b/C2c1, Cas12c/C2c3, Cas12d/CasY, Cas12e/CasX, Cas12g, Cas12h, Cas12i, Cas12j/CasΘ, Cpf1, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csx11, Csf1, Csf2, CsO, Csf4, Csd1, Csd2, Cst1, Cst2, Csh1, Csh2, Csa1, Csa2, Csa3, Csa4, Csa5, Type II Cas effector proteins, Type V Cas effector proteins, Type VI Cas effector proteins, CARF, DinG, homologues thereof, or modified or engineered versions thereof. Other nucleic acid programmable DNA binding proteins are also within the scope of this disclosure, although they may not be specifically listed in this disclosure. See, e.g., Makarova et al. “Classification and Nomenclature of CRISPR-Cas Systems: Where from Here?” CRISPR J. 2018 October; 1:325-336. doi: 10.1089/crispr.2018.0033; Yan et al., “Functionally diverse type V CRISPR-Cas systems” Science. 2019 Jan. 4;363 (6422): 88-91. doi: 10.1126/science.aav7271, the entire contents of each are hereby incorporated by reference. Exemplary nucleic acid programmable DNA binding proteins and nucleic acid sequences encoding nucleic acid programmable DNA binding proteins are provided in the Sequence Listing as SEQ ID NOs: 197-245, 254-260, and 378.

The terms “nucleobase editing domain” or “nucleobase editing protein,” as used herein, refers to a protein or enzyme that can catalyze a nucleobase modification in RNA or DNA, such as cytosine (or cytidine) to uracil (or uridine) or thymine (or thymidine), and adenine (or adenosine) to hypoxanthine (or inosine) deaminations, as well as non-templated nucleotide additions and insertions. In some embodiments, the nucleobase editing domain is a deaminase domain (e.g., an adenine deaminase or an adenosine deaminase; or a cytidine deaminase or a cytosine deaminase).

As used herein, “obtaining” as in “obtaining an agent” includes synthesizing, purchasing, or otherwise acquiring the agent.

By “subject” or “patient” is meant a mammal, including, but not limited to, a human or non-human mammal. In embodiments, the mammal is a bovine, equine, canine, ovine, rabbit, rodent, nonhuman primate, or feline. In an embodiment, “patient” refers to a mammalian subject with a higher than average likelihood of developing a disease or a disorder. Exemplary patients can be humans, non-human primates, cats, dogs, pigs, cattle, cats, horses, camels, llamas, goats, sheep, rodents (e.g., mice, rabbits, rats, or guinea pigs) and other mammalians that can benefit from the therapies disclosed herein. Exemplary human patients can be male and/or female.

“Patient in need thereof” or “subject in need thereof” is referred to herein as a patient diagnosed with, at risk or having, predetermined to have, or suspected of having a disease or disorder.

The terms “pathogenic mutation”, “pathogenic variant”, “disease causing mutation”, “disease causing variant”, “deleterious mutation”, or “predisposing mutation” refers to a genetic alteration or mutation that is associated with a disease or disorder or that increases an individual's susceptibility or predisposition to a certain disease or disorder. In some embodiments, the pathogenic mutation comprises at least one wild-type amino acid substituted by at least one pathogenic amino acid in a protein encoded by a gene. In some embodiments, the pathogenic mutation is in a terminating region (e.g., stop codon). In some embodiments, the pathogenic mutation is in a non-coding region (e.g., intron, promoter, etc.).

The terms “protein”, “peptide”, “polypeptide”, and their grammatical equivalents are used interchangeably herein, and refer to a polymer of amino acid residues linked together by peptide (amide) bonds. A protein, peptide, or polypeptide can be naturally occurring, recombinant, or synthetic, or any combination thereof.

The term “fusion protein” as used herein refers to a hybrid polypeptide which comprises protein domains from at least two different proteins.

The term “recombinant” as used herein in the context of proteins or nucleic acids refers to proteins or nucleic acids that do not occur in nature but are the product of human engineering. For example, in some embodiments, a recombinant protein or nucleic acid molecule comprises an amino acid or nucleotide sequence that comprises at least one, at least two, at least three, at least four, at least five, at least six, or at least seven mutations as compared to any naturally occurring sequence.

By “reduces” is meant a negative alteration of at least 10%, 25%, 50%, 75%, or 100%.

By “reference” is meant a standard or control condition. In one embodiment, the reference is a wild-type or healthy cell. In other embodiments and without limitation, a reference is an untreated cell that is not subjected to a test condition, or is subjected to placebo or normal saline, medium, buffer, and/or a control vector that does not harbor a polynucleotide of interest. The reference can be a cell or subject with a pathogenic mutation in a transhyretin (TTR) polynucleotide sequence and/or a transthyretin (TTR) polypeptide sequence. A reference can be a subject or cell with an amyloidosis (e.g., a transthyretin amyloidosis) or a subject or cell without an amyloidosis.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, at least about 20 amino acids, at least about 25 amino acids, about 35 amino acids, about 50 amino acids, or about 100 amino acids. For nucleic acids, the length of the reference nucleic acid sequence will generally be at least about 50 nucleotides, at least about 60 nucleotides, at least about 75 nucleotides, about 100 nucleotides or about 300 nucleotides or any integer thereabout or therebetween. In some embodiments, a reference sequence is a wild-type sequence of a protein of interest. In other embodiments, a reference sequence is a polynucleotide sequence encoding a wild-type protein.

The term “RNA-programmable nuclease,” and “RNA-guided nuclease” refer to a nuclease that forms a complex with (e.g., binds or associates with) one or more RNA(s) that is not a target for cleavage. In some embodiments, an RNA-programmable nuclease, when in a complex with an RNA, may be referred to as a nuclease-RNA complex. Typically, the bound RNA(s) is referred to as a guide RNA (gRNA). In some embodiments, the RNA-programmable nuclease is the (CRISPR-associated system)Cas9 endonuclease, for example, Cas9 (Csnl) from Streptococcus pyogenes (e.g., SEQ ID NO: 197), Cas9 from Neisseria meningitidis (NmeCas9; SEQ ID NO: 208), Nme2Cas9 (SEQ ID NO: 209), Streptococcus constellatus (ScoCas9), or derivatives thereof (e.g., a sequence with at least about 85% sequence identity to a Cas9, such as Nme2Cas9 or spCas9).

Amino acids generally can be grouped into classes according to the following common side-chain properties:
(1) hydrophobic: Norleucine, Met, Ala, Val, Leu, He;
(2) neutral hydrophilic: Cys, Ser, Thr, Asn, Gin;
(3) acidic: Asp, Glu;
(4) basic: His, Lys, Arg;
(5) residues that influence chain orientation: Gly, Pro;
(6) aromatic: Trp, Tyr, Phe.

In some embodiments, conservative substitutions can involve the exchange of a member of one of these classes for another member of the same class. In some embodiments, non-conservative amino acid substitutions can involve exchanging a member of one of these classes for another class.

The term “single nucleotide polymorphism (SNP)” is a variation in a single nucleotide that occurs at a specific position in the genome, where each variation is present to some appreciable degree within a population (e.g., >1%). SNPs can fall within coding regions of genes, non-coding regions of genes, or in the intergenic regions (regions between genes). In some embodiments, SNPs within a coding sequence do not necessarily change the amino acid sequence of the protein that is produced, due to degeneracy of the genetic code. SNPs in the coding region are of two types: synonymous and nonsynonymous SNPs. Synonymous SNPs do not affect the protein sequence, while nonsynonymous SNPs change the amino acid sequence of protein. The nonsynonymous SNPs are of two types: missense and nonsense. SNPs that are not in protein-coding regions can still affect gene splicing, transcription factor binding, messenger RNA degradation, or the sequence of noncoding RNA. Gene expression affected by this type of SNP is referred to as an eSNP(expression SNP) and can be upstream or downstream from the gene. A single nucleotide variant (SNV) is a variation in a single nucleotide without any limitations of frequency and can arise in somatic cells. A somatic single nucleotide variation can also be called a single-nucleotide alteration.

By “specifically binds” is meant a nucleic acid molecule, polypeptide, polypeptide/polynucleotide complex, compound, or molecule that recognizes and binds a polypeptide and/or nucleic acid molecule of the disclosure, but which does not substantially recognize and bind other molecules in a sample, for example, a biological sample.

By “substantially identical” is meant a polypeptide or nucleic acid molecule exhibiting at least 50% identity to a reference amino acid sequence. In one embodiment, a reference sequence is a wild-type amino acid or nucleic acid sequence. In another embodiment, a reference sequence is any one of the amino acid or nucleic acid sequences described herein. In one embodiment, such a sequence is at least about 60%, 80%, 85%, 90%, 95%, 96%, 97%, 98%, 99%, 99.9%, or even 99.99%, identical at the amino acid level or nucleic acid level to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

Nucleic acid molecules useful in the methods of the disclosure include any nucleic acid molecule that encodes a polypeptide of the disclosure or a functional fragment thereof. Such nucleic acid molecules need not be 100% identical with an endogenous nucleic acid sequence but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the disclosure include any nucleic acid molecule that encodes a polypeptide of the disclosure or a functional fragment thereof. Such nucleic acid molecules need not be 100% identical with an endogenous nucleic acid sequence but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

The term “target site” refers to a nucleotide sequence or nucleobase of interest within a nucleic acid molecule that is modified. In embodiments, the modification is deamination of a base. The deaminase can be a cytidine or an adenine deaminase. The fusion protein or base editing complex comprising a deaminase may comprise a dCas9-adenosine deaminase fusion protein, a Cas12b-adenosine deaminase fusion, or a base editor disclosed herein.

As used herein, the terms “treat,” treating,” “treatment,” and the like refer to reducing or ameliorating a disorder and/or symptoms associated therewith or obtaining a desired pharmacologic and/or physiologic effect. It will be appreciated that, although not precluded, treating a disorder or condition does not require that the disorder, condition or symptoms associated therewith be completely eliminated. In some embodiments, the effect is therapeutic, i.e., without limitation, the effect partially or completely reduces, diminishes, abrogates, abates, alleviates, reduces the intensity of, or cures a disease and/or adverse symptom attributable to the disease. In some embodiments, the effect is preventative, i.e., the effect protects or prevents an occurrence or reoccurrence of a disease or condition. To this end, the presently disclosed methods comprise administering a therapeutically effective amount of a composition as described herein.

By “transthyretin (TTR) polypeptide” is meant a polypeptide or fragment thereof having at least about 95% amino acid sequence identity to an amino acid sequence provided at NCBI Reference Sequence No. NP_000362.1, or a fragment thereof, that binds an anti-TTR antibody. In some embodiments, a TTR polypeptide or fragment thereof has holo-retinol-binding protein (RBP) and/or thyroxine (T4) transport activity. Typically, amino acid locations for mutations to the TTR polypeptide are numbered with reference to the mature TTR polypeptide (i.e., the TTR polypeptide without a signal sequence). In embodiments, TTR is capable of forming a tetramer. An exemplary TTR polypeptide sequence follows (the signal peptide sequence is in bold; therefore, the mature TTR polypeptide corresponds to amino acids 21 to 147 of the following sequence):

(SEQ ID NO: 464)
MASHRLLLLCLAGLVFVSEAGPTGTGESKCPLMVKVLDAVRGSPAINVAV
HVFRKAADDTWEPFASGKTSESGELHGLTTEEEFVEGIYKVEIDTKSYWK
ALGISPFHEHAEVVFTANDSGPRRYTIAALLSPYSYSTTAVVTNPKE.

By “transthyretin (TTR) polynucleotide” is meant a nucleic acid molecule that encodes a TTR, as well as the introns, exons, 3′ untranslated regions, 5′ untranslated regions, and regulatory sequences associated with its expression, or fragments thereof. In embodiments, the regulatory sequence is a promoter region. In embodiments, a TTR polynucleotide is the genomic sequence, cDNA, mRNA, or gene associated with and/or required for TTR expression. An exemplary TTR polynucleotide sequence (corresponding to Consensus Coding Sequence (CCDS) No. 11899.1) is provided below. Further exemplary TTR polynucleotide sequences include Gene Ensembl ID: ENSG00000118271 and Transcript Ensembl ID: ENST00000237014.8.

(SEQ ID NO: 465)
ATGGCTTCTCATCGTCTGCTCCTCCTCTGCCTTGCTGGACTGGTATTTGT
GTCTGAGGCTGGCCCTACGGGCACCGGTGAATCCAAGTGTCCTCTGATGG
TCAAAGTTCTAGATGCTGTCCGAGGCAGTCCTGCCATCAATGTGGCCGTG
CATGTGTTCAGAAAGGCTGCTGATGACACCTGGGAGCCATTTGCCTCTGG
GAAAACCAGTGAGTCTGGAGAGCTGCATGGGCTCACAACTGAGGAGGAAT
TTGTAGAAGGGATATACAAAGTGGAAATAGACACCAAATCTTACTGGAAG
GCACTTGGCATCTCCCCATTCCATGAGCATGCAGAGGTGGTATTCACAGC
CAACGACTCCGGCCCCCGCCGCTACACCATTGCCGCCCTGCTGAGCCCCT
ACTCCTATTCCACCACGGCTGTCGTCACCAATCCCAAGGAATGA.

A further exemplary TTR polynucleotide sequence is provided at NCBI Reference Sequence No. NG_009490.1 and follows (where exons encoding the TTR polypeptide are in bold, introns are in italics, and exemplary promoter regions are indicated by the combined underlined and bold-underlined text (promoter positions −1 to −177) and by the bold-underlined text (promoter positions −106 to −176); further exemplary promoter regions are shown in FIGS. 37A, 37B, 40A, and 40B):

(SEQ ID NO: 466)
TTATGTGTTTATTCAACAATGGCGGAGGAGAGGCATGCCAGATAAGGCAGACACGGGCATTC
CAAACACAAGAAAGGTATGTGCTGCAGAGAAGTCAGATAACTTTCCTAGGCTCTCCTGCAGT
CCGGATGAAATACTCTCAAAAAATTAGCCCGGGCCCTTTGCTCCAATTTTTCGCTTACCTAG
CAACCATCTAACTATTAATTAAATTGGTATTATGGTTTTAACATGAATCTTTTATGATTTGC
TTACCATTAATCAAACCCCCGAGGCTTATTCACCTCAAGGGGAGCTGACAAAGTTGAATTAT
TCAACCTGCAAAGATCCAGGGCCCCCAAATACTGTCATTTCCACTCTCCCCTAACCCCCACC
ATGAGGCCCAGTCTCAGCACTCGGCCAGCCTATGCCCAACTCGGGGTAATCAGCTTAGACAT
ATTAATATTAGTGGGCATTTCAGTATCAACAGATCACTGTCTAGCAGCTGACAGGCACCCTC
AGAAAATAAACCAAGAAGAAAGGGTTTATCTATAATATCAAAATTTTTCATAGATAAACCTG
CCCATTATAAGGAAGAGGGCAGAAGAACCCTAAACTAAGAGCCAGGCAACTTGTTCATTAAT
CACAGCATATTCCATAGAAGGAGGAGAAATGTTGTCATCAAATTCATCTTTTTCACCTCAAT
TAAACATCTATGCTACAGCTCCACAGTCAGATTGAGAGGAAAAACAGTACGTAGCTAAGAAA
AGACATAGACTTGTAACTGAAATGCTTCACTGGTGCTCCTTTTGTTTTAAGGCATTGGATCT
TCATAGCTACTGATCGTGCCCAAGCACACAGTATCTGCAGCAACCACTTAGGCCTCCAGGAA
TGTGGTGACCATTGACCCTAATTCATTCCCCTTCATGGATCCTATGTAACCATCCTCCAAAA
AGAGCTTTCGCAAACTCAAATAAACACAGGAAAGGAAGACCTTCTTATCTTTGAGAGTATAT
GTTTAGCCCTATAACCCTCTCTTATCATAAATTGCTTCTTAGGCAAGAAACACTGGATTTTT
CTTGTATTTGTCATTGCCATTGGTTCCATACAAGCATTCATTTAACAAATAATACATTCCAT
CTCCGTTTTTTGCTTTTTCCTTCATGCTTCGGCCTTGGTTTTCTCTCACCTAAAACACTACA
AGCTTCTTCTCCCAGAGCTCTCACTTTGACTCCAGACTACCTACATTTAATCTTTAATTCTC
TACCAAAATTTTCTCTTATCTTTATATCTTTTTAATTTTAATTTTATTTTTTGTGGATACAT
AGTAGGTGTATATATTTATGGGGTACATGCAATGTTTTAATACAGGCATGCAATGTGAAATA
AGGACATCATGGAGAATGGGGTATCCATCTCCTCAAGCATTTATCTTTTCAGTTACAAACAA
TCCAATTACACTCTTTATTTTAAAATATACAATTATTAATTATAGTCACTCTGTTGTGCTAT
CAAATAGTAGATCTTATTCTTTCTATTTTTTGTACCCTCTCGTCTTTTTATATTAAAAAATA
ATCTTTATCTCTGTAAGCTTCATCAGTGATTTTCCAATGAAATTTAGGATCTTCTCTATACC
CTGAATTGCCTTACTTTCTCCCCACTTCCTTGTCTTATTCAAATGCAGATTTCATTAATGAT
TTCCAGATCAATGATAGTTCAGAAAGCAAGCAAGTCAAAGTGACCAAGGGCATGGCCTGAAA
ACTGTTCTAAGAGAGGAATTTACAGAACAACTATTAAATGATGTCAATAGGATTGTATTAGT
CCGTTTTCATACGGCTATAAAGAACTGCCTGAGACTGGGTAATTTATAAAGGAAAGAGGTTT
AATTGACTCACAGTTCAGCACAACTGGGCAGGCCTCAGGAAACTTACAATCATGGTAGAAGG
TGAAGGGGAAGCAAAGCACCTTCCTCACAAGGCGTCAGGAAGAAGTGCCAAGCAAAGGGGGA
AAAGCCCCTTGTAAAACTACCAGAACCTGTGAGAACTCAATCACTATCACAAGAACAGCATG
AGGGAACCGCCCCTCGTGATTCAATTACCTCCACCTGGTCTCTCCCTTGACACATGGGGATT
ATGGGTGTTACAATTCAAGATGAGATTTGGGTGGGGACACAAAGCCTAACCATATCAAGGAT
CAAGTGGTGGGTTGAAACTAACAGGATGAGATATATCAGATACAAACACAGGGTCCCATATT
TGGGTTAAAATTCATAAATGATCAAAGCACAGGATGACAGATAATATAGGTCATTTTAGATT
ATTGTGGCCAACAGATCACAGTGGGTAGTGTTATGACGAAGGGAGGGTCACAGTTACTACAG
TTACAGATGGATTCTGGGTACAACATTTGCACTAAAGTGCCTTTGCCAAGGGAGGCAACAGT
CTCGACATCCTGTGGCCTGATCTACTTCAGGGACTGTGTCTTGTTCAGAGCATCACATTTGA
AGAGAACTTTGACCAAGGGGAATATGCCAGAAAAGGAAGTTCGGGATGCTGAGGATCTTAGG
AACTATGTCTAAACAAGATTCATTCACAGAAGTGGGAATGTCTATTTGGCAAAAAGAAAATA
CTACTTACATGGCTGTTGGAAGACCAGCAATCACAAACTCAGTTTTTCAAAAGGCTGGGCAG
AAACACAGATGAAAGAAACAGGCCATGTTTAAGAAAAGATAAAAGCTCACGCATGATATGCC
ACTAGAGAATCACCTAGCCTCAGTGTTGGCGGGGAGGCCTGGGGAGTCTTGATGTCTGAGAG
TGACATTCTGATGATCACTGTCATGTGTAAATGTTGGCCTAAAGCTGCCAATATTTTTGATT
TAAGAGAAGCAAGAAATGCAAATTTTTATGCAGCATGTCTCAATTTTTAATTTTGGCAACTA
TTACAAAATGTTTAAAGAGACTCTGTGCAGCCCAAATATAACATATCTATGGGCTGATGGCA
GCCCAGCGTTGCCAGTTCACAGGGTCTACAAGAGATGATTCTTAGTTTCAACAGGGTGCAGT
GCTGAAACGCGTGCACAGTAGATTTTGCTTCGGTTATGAAAGAACTTCCAAATATTTATGAT
TCATAGCCAGAGAAAAGGCTCTCTATCCAGGTTCTGAACAATAGGAAATCATCAAGAGGATA
TTGGATGACAATATATGAAAGATGTTATTTGAGAAAGGATTCTCTCCTGAGGCATAGATGTT
GAACCAAATTCTATTAGTTATGCTTTTACAGCAAGATAGTGGTTTACAGCTTACAAAAGGCT
TGTACATCCTCTCATATTAAAAGTTATTAGAACAGTCCTTTGAAGTAGAAAAGTAGGCATTT
CTATTTTACAAACGAGTTGGCCGAGTATCTGAGATAGTAGATAACTCATAGAAGGTCATCCG
GGAAACGGGGCAGCAGAACTGGGATCGAATGACTCTGGTCATCCAACTCCAAATGCAAAAGT
CTTTCTGCTGCTGCTTCCTAGTTAAACTCTAAGGGTCTAAGACTCCATTCCTAGTTATGGTC
TCAACTACATTTGCTCATTGCTGTGAGGGGTCAACCCACCTCCCGGAGTCCTCTCCTGCACA
TTCTCATGTTCCTGAAAGGCTTTTCTGTCCCTTCCACTACTCCCTGTAAGCTCCTGTGCTTC
ACAATTTCTTGTTGAATTTTTTCTAATCTGACTCTATCAGTTATGGGAATGTTCCCTCAATT
CTTAGTGCTCCAAACCGGACTTGCTCTTGGCTTGTATTTGTCCAAAATATTTGTCTTCTCTA
TGTTTTCTACATGTTTGTCTTATAAGGACAAAAACCTGCCTTAGTTTATCCATGAACAAAGC
CACGCATGCTAGTGGACACACACACACATGCGCGTGCGCGCGCACACACACACACACACACA
TACACACAGAGACTTTGTATGTGAGTAATGAATCATCAAATCATCATAATTTCTGGACTTGT
ATTAATAAGTCGGCCAGGAGGAAAAGAATCTGCTGTCAATCATGGCTTCTGGTTCTCACAGT
CATCTCTACTTTCTTCCAGCAAGTTTGGTTCTGTCAAAAACCAGCTGTCAGCCTTGTTCCTG
CATGCCCAATGCAGAAGAGTCAGTAAAGAAGATTTGGTTCTCTGTATTTCAGGGGCATCAAT
GCCAGGTTGAAATATGCCATTCTGGCCCAGCTCAGTGGCTCACACGTGTAATCCCAGCACTT
TGGAAGGCCAAAGCGGGTGGATTGCTTGAGCTCAGGAGTTCGAGACCAGCCTGGGCAAGAGG
CTGAGGTGGGAGGATGACCTGAGCCCGGGAGGTCAAGGCTGCAGCGAGCTGTGATCGTGCCA
CTGCACTCGAGCCAGGGCGTTGGAGTGAGACCCTGTCAAAAAAAAAAAAAAAAAGGAAGGAA
AAAAGGAAGGAAGGAAGGGAGGGAGGGAAGATGCCATTCTTAGATTGAAGTGGACTTTATCT
GGGCAGAACACACACACACATACACACATGCACACACACATTGTGGAGAAATTGCTGACTAA
GCAAAGCTTCCAAATGACTTAGTTTGGCTAAAATGTAGGCTTTTAAAAATGTGAGCACTGCC
AAGGGTTTTTCCTTGTTGACCCATGGATCCATCAAGTGCAAACATTTTCTAATGCACTATAT
TTAAGCCTGTGCAGCTAGATGTCATTCAACATGAAATACATTATTACAACTTGCATCTGTCT
AAAATCTTGCATCTAAAATGAGAGACAAAAAATCTATAAAAATGGAAAACATGCATAGAAAT
ATGTGAGGGAGGAAAAAATTACCCCCAAGAATGTTAGTGCACGCAGTCACACAGGGAGAAGA
CTATTTTTGTTTTGTTTTGATTGTTTTGTTTTGTTTTGGTTGTTTTGTTTTGGTGACCTAAC
TGGTCAAATGACCTATTAAGAATATTTCATAGAACGAATGTTCCGATGCTCTAATCTCTCTA
GACAAGGTTCATATTTGTATGGGTTACTTATTCTCTCTTTGTTGACTAAGTCAATAATCAGA
ATCAGCAGGTTTGCAGTCAGATTGGCAGGGATAAGCAGCCTAGCTCAGGAGAAGTGAGTATA
AAAGCCCCAGGCTGGGAGCAGCCATCACAGAAGTCCACTCATTCTTGGCAGGATGGCTTCTC
ATCGTCTGCTCCTCCTCTGCCTTGCTGGACTGGTATTTGTGTCTGAGGCTGGCCCTACGGTG
AGTGTTTCTGTGACATCCCATTCCTACATTTAAGATTCACGCTAAATGAAGTAGAAGTGACT
CCTTCCAGCTTTGCCAACCAGCTTTTATTACTAGGGCAAGGGTACCCAGCATCTATTTTTAA
TATAATTAATTCAAACTTCAAAAAGAATGAAGTTCCACTGAGCTTACTGAGCTGGGACTTGA
ACTCTGAGCATTCTACCTCATTGCTTTGGTGCATTAGGTTTGTAATATCTGGTACCTCTGTT
TCCTCAGATAGATGATAGAAATAAAGATATGATATTAAGGAAGCTGTTAATACTGAATTTTC
AGAAAAGTATCCCTCCATAAAATGTATTTGGGGGACAAACTGCAGGAGATTATATTCTGGCC
CTATAGTTATTCAAAACGTATTTATTGATTAATCTTTAAAAGGCTTAGTGAACAATATTCTA
GTCAGATATCTAATTCTTAAATCCTCTAGAAGAATTAACTAATACTATAAAATGGGTCTGGA
TGTAGTTCTGACATTATTTTATAACAACTGGTAAGAGGGAGTGACTATAGCAACAACTAAAA
TGATCTCAGGAAAACCTGTTTGGCCCTATGTATGGTACATTACATCTTTTCAGTAATTCCAC
TCAAATGGAGACTTTTAACAAAGCAACTGTTCTCAGGGGACCTATTTTCTCCCTTAAAATTC
ATTATACACATCCCTGGTTGATAGCAGTGTGTCTGGAGGCAGAAACCATTCTTGCTTTGGAA
ACAATTACGTCTGTGTTATACTGAGTAGGGAAGCTCATTAATTGTCGACACTTACGTTCCTG
ATAATGGGATCAGTGTGTAATTCTTGTTTCGCTCCAGATTTCTAATACCACAAAGAATAAAT
CCTTTCACTCTGATCAATTTTGTTAACTTCTCACGTGTCTTCTCTACACCCAGGGCACCGGT
GAATCCAAGTGTCCTCTGATGGTCAAAGTTCTAGATGCTGTCCGAGGCAGTCCTGCCATCAA
TGTGGCCGTGCATGTGTTCAGAAAGGCTGCTGATGACACCTGGGAGCCATTTGCCTCTGGGT
AAGTTGCCAAAGAACCCTCCCACAGGACTTGGTTTTATCTTCCCGTTTGCCCCTCACTTGGT
AGAGAGAGGCTCACATCATCTGCTAAAGAATTTACAAGTAGATTGAAAAACGTAGGCAGAGG
TCAAGTATGCCCTCTGAAGGATGCCCTCTTTTTGTTTTGCTTAGCTAGGAAGTGACCAGGAA
CCTGAGCATCATTTAGGGGCAGACAGTAGAGAAAAGAAGGAATCAGAACTCCTCTCCTCTAG
CTGTGGTTTGCAACCCTTTTGGGTCACAGAACACTTTATGTAGGTGATGAAAAGTAAACATT
CTATGCCCAGAAAAAATGCACAGATACACACACATACAAAATCATATATGTGATTTTAGGAG
TTTCACAGATTCCCTGGTGTCCCTGGGTAACACCAAAGCTAAGTGTCCTTGTCTTAGAATTT
TAGGAAAAGGTATAATGTGTATTAACCCATTAACAAAAGGAAAGGAATTCAGAAATATTATT
AACCAGGCATCTGTCTGTAGTTAATATGGATCACCCAAAACCCAAGGCTTTTGCCTAATGAA
CACTTTGGGGCACCTACTGTGTGCAAGGCTGGGGGCTGTCAAGCTCAGTTAAAAAAAAAAAG
ATAGAAGAGATGGATCCATGAGGCAAAGTACAGCCCCAGGCTAATCCCACGATCACCCGACT
TCATGTCCAAGAGTGGCTTCTCACCTTCATTAGCCAGTTCACAATTTTCATGGAGTTTTTCT
ACCTGCACTAGCAAAAACTTCAAGGAAAATACATATTAATAAATCTAAGCAAAGTGACCAGA
AGACAGAGCAATCAGGAGACCCTTTGCATCCAGCAGAAGAGGAACTGCTAAGTATTTACATC
TCCACAGAGAAGAATTTCTGTTGGGTTTTAATTGAACCCCAAGAACCACATGATTCTTCAAC
CATTATTGGGAAGATCATTTTCTTAGGTCTGGTTTTAACTGGCTTTTTATTTGGGAATTCAT
TTATGTTTATATAAAATGCCAAGCATAACATGAAAAGTGGTTACAGGACTATTCTAAGGGAG
AGACAGAATGGACACCAAAAATATTCCAATGTTCTTGTGAATCTTTTCCTTGCACCAGGACA
AAAAAAAAAAGAAGTGAAAAGAAGAAAGGAGGAGGGGCATAATCAGAGTCAGTAAAGACAAC
TGCTATTTTTATCTATCGTAGCTGTTGCAGTCAAATGGGAAGCAATTTCCAACATTCAACTA
TGGAGCTGGTACTTACATGGAAATAGAAGTTGCCTAGTGTTTGTTGCTGGCAAAGAGTTATC
AGAGAGGTTAAATATATAAAAGGGAAAAGAGTCAGATACAGGTTCTTCTTCCTACTTTAGGT
TTTCCACTGTGTGTGCAAATGATACTCCCTGGTGGTGTGCAGATGCCTCAAAGCTATCCTCA
CACCACAAGGGAGAGGAGCGAGATCCTGCTGTCCTGGAGAAGTGCAGAGTTAGAACAGCTGT
GGCCACTTGCATCCAATCATCAATCTTGAATCACAGGGACTCTTTCTTAAGTAAACATTATA
CCTGGCCGGGCACGGTGGCTCACGCCTGTAATCCCAGCACTTTGGGATGCCAAAGTGGGCAT
ATCATCTGAGGTCAGGAGTTCAAGACCAGCCTGGCCAACATGGCAAAACTCCGTCTTTATGA
AAAATACAAAAATTAGCCAGGCATGGTGGCAGGCGCCTGTAATCCCAGCTAATTGGGAGGCT
GAGGCTGGAGAATCCCTTGAATCTAGGAGGCAGAGGTTGCAGTGAGCTGAGATCGTGCCATT
GCACTCCAGCCTGGGTGACAAGAGTAAAACTCTGTCTCAAAAAAAAAAAATTATACCTACAT
TCTCTTCTTATCAGAGAAAAAAATCTACAGTGAGCTTTTCAAAAAGTTTTTACAAACTTTTT
GCCATTTAATTTCAGTTAGGAGTTTTCCCTACTTCTGACTTAGTTGAGGGGAAATGTTCATA
ACATGTTTATAACATGTTTATGTGTGTTAGTTGGTGGGGGTGTATTACTTTGCCATGCCATT
TGTTTCCTCCATGCGTAACTTAATCCAGACTTTCACACCTTATAGGAAAACCAGTGAGTCTG
GAGAGCTGCATGGGCTCACAACTGAGGAGGAATTTGTAGAAGGGATATACAAAGTGGAAATA
GACACCAAATCTTACTGGAAGGCACTTGGCATCTCCCCATTCCATGAGCATGCAGAGGTGAG
TATACAGACCTTCGAGGGTTGTTTTGGTTTTGGTTTTTGCTTTTGGCATTCCAGGAAATGCA
CAGTTTTACTCAGTGTACCACAGAAATGTCCTAAGGAAGGTGATGAATGACCAAAGGTTCCC
TTTCCTATTATACAAGAAAAAATTCACAACACTCTGAGAAGCAAATTTCTTTTTGACTTTGA
TGAAAATCCACTTAGTAACATGACTTGAACTTACATGAAACTACTCATAGTCTATTCATTCC
ACTTTATATGAATATTGATGTATCTGCTGTTGAAATAATAGTTTATGAGGCAGCCCTCCAGA
CCCCACGTAGAGTGTATGTAACAAGAGATGCACCATTTTATTTCTCGAAAACCCGTAACATT
CTTCATTCCAAAACACATCTGGCTTCTCGGAGGTCTGGACAAGTGATTCTTGGCAACACATA
CCTATAGAGACAATAAAATCAAAGTAATAATGGCAACACAATAGATAACATTTACCAAGCAT
ACACCATGTGGCAGACACAATTATAAGTGTTTTCCATATTTAACCTACTTAATCCTCAGGAA
TAAGCCACTGAGGTCAGTCCTATTATTATCCCCATCTTATAGATGAAGAAAATGAGGCACCA
GGAAGTCAAATAACTTGTCAAAGGTCACAAGACTAGGAAATACACAAGTAGAAATGTTTACA
ATTAAGGCCCAGGCTGGGTTTGCCCTCAGTTCTGCTATGCCTCGCATTATGCCCCAGGAAAC
TTTTTCCCTTGTGAAAGCCAAGCTTAAAAAAAGAAAAGCCACATTTGTAACGTGCTCTGTTC
CCCTGCCTATGGTGAGGATCTTCAAACAGTTATACATGGACCCAGTCCCCCTGCCTTCTCCT
TAATTTCTTAAGTCATTTGAAACAGATGGCTGTCATGGAAATAGAATCCAGACATGTTGGTC
AGAGTTAAAGATCAACTAATTCCATCAAAAATAGCTCGGCATGAAAGGGAACTATTCTCTGG
CTTAGTCATGGATGAGACTTTCAATTGCTATAAAGTGGTTCCTTTATTAGACAATGTTACCA
GGGAAACAACAGGGGTTTGTTTGACTTCTGGGGCCCACAAGTCAACAAGAGAGCCCCATCTA
CCAAGGAGCATGTCCCTGACTACCCCTCAGCCAGCAGCAAGACATGGACCCCAGTCAGGGCA
GGAGCAGGGTTTCGGCGGCGCCCAGCACAAGACATTGCCCCTAGAGTCTCAGCCCCTACCCT
CGAGTAATAGATCTGCCTACCTGAGACTGTTGTTTGCCCAAGAGCTGGGTCTCAGCCTGATG
GGAACCATATAAAAAGGTTCACTGACATACTGCCCACATGTTGTTCTCTTTCATTAGATCTT
AGCTTCCTTGTCTGCTCTTCATTCTTGCAGTATTCATTCAACAAACATTAAAAAAAAAAAAA
AGCATTCTATGTGTGGAACACTCTGCTAGATGCTGTGGATTTAGAAATGAAAATACATCCCG
ACCCTTGGAATGGAAGGGAAAGGACTGAAGTAAGACAGATTAAGCAGGACCGTCAGCCCAGC
TTGAAGCCCAGATAAATACGGAGAACAAGAGAGAGCGAGTAGTGAGAGATGAGTCCCAATGC
CTCACTTTGGTGACGGGTGCGTGGTGGGCTTCATGCAGCTTCTTCTGATAAATGCCTCCTTC
AGAACTGGTCAACTCTACCTTGGCCAGTGACCCAGGTGGTCATAGTAGATTTACCAAGGGAA
AATGGAAACTTTTATTAGGAGCTCTTAGGCCTCTTCACTTCATGGATTTTTTTTTCCTTTTT
TTTTGAGATGGAGTTTTGCCCTGTCACCCAGGCTGGAATGCAGTGGTGCAATCTCAGCTCAC
TGCAACCTCCGCCTCCCAGGTTCAAGCAATTCTCCTGCCTCAGCCTCCCGAGTAGCTGGGAC
TACAGGTGTGCGCCACCACACCAGGCTAATTTTTGTATTTTTTGTAAAGACAGGTTTTCACC
ACGTTGGCCAGGCTGGTCTGAACTCCAGACCTCAGGTGATTCACCTGTCTCAGCCTCCCAAA
GTGCTGGGATTACAGGTGTGAGCCACCGTGCCCGGCTACTTCATGGATTTTTGATTACAGAT
TATGCCTCTTACAATTTTTAAGAAGAATCAAGTGGGCTGAAGGTCAATGTCACCATAAGACA
AAAGACATTTTTATTAGTTGATTCTAGGGAATTGGCCTTAAGGGGAGCCCTTTCTTCCTAAG
AGATTCTTAGGTGATTCTCACTTCCTCTTGCCCCAGTATTATTTTTGTTTTTGGTATGGCTC
ACTCAGATCCTTTTTTCCTCCTATCCCTAAGTAATCCGGGTTTCTTTTTCCCATATTTAGAA
CAAAATGTATTTATGCAGAGTGTGTCCAAACCTCAACCCAAGGCCTGTATACAAAATAAATC
AAATTAAACACATCTTTACTGTCTTCTACCTCTTTCCTGACCTCAATATATCCCAACTTGCC
TCACTCTGAGAACCAAGGCTGTCCCAGCACCTGAGTCGCAGATATTCTACTGATTTGACAGA
ACTGTGTGACTATCTGGAACAGCATTTTGATCCACAATTTGCCCAGTTACAAAGCTTAAATG
AGCTCTAGTGCATGCATATATATTTCAAAATTCCACCATGATCTTCCACACTCTGTATTGTA
AATAGAGCCCTGTAATGCTTTTACTTCGTATTTCATTGCTTGTTATACATAAAAATATACTT
TTCTTCTTCATGTTAGAAAATGCAAAGAATAGGAGGGTGGGGGAATCTCTGGGCTTGGAGAC
AGGAGACTTGCCTTCCTACTATGGTTCCATCAGAATGTAGACTGGGACAATACAATAATTCA
AGTCTGGTTTGCTCATCTGTAAATTGGGAAGAATGTTTCCAGCTCCAGAATGCTAAATCTCT
AAGTCTGTGGTTGGCAGCCACTATTGCAGCAGCTCTTCAATGACTCAATGCAGTTTTGCATT
CTCCCTACCTTTTTTTTCTAAAACCAATAAAATAGATACAGCCTTTAGGCTTTCTGGGATTT
CCCTTAGTCAAGCTAGGGTCATCCTGACTTTCGGCGTGAATTTGCAAAACAAGACCTGACTC
TGTACTCCTGCTCTAAGGACTGTGCATGGTTCCAAAGGCTTAGCTTGCCAGCATATTTGAGC
TTTTTCCTTCTGTTCAAACTGTTCCAAAATATAAAAGAATAAAATTAATTAAGTTGGCACTG
GACTTCCGGTGGTCAGTCATGTGTGTCATCTGTCACGTTTTTCGGGCTCTGGTGGAAATGGA
TCTGTCTGTCTTCTCTCATAGGTGGTATTCACAGCCAACGACTCCGGCCCCCGCCGCTACAC
CATTGCCGCCCTGCTGAGCCCCTACTCCTATTCCACCACGGCTGTCGTCACCAATCCCAAGG
AATGAGGGACTTCTCCTCCAGTGGACCTGAAGGACGAGGGATGGGATTTCATGTAACCAAGA
GTATTCCATTTTTACTAAAGCAGTGTTTTCACCTCATATGCTATGTTAGAAGTCCAGGCAGA
GACAATAAAACATTCCTGTGAAAGGCACTTTTCATTCCACTTTAACTTGATTTTTTAAATTC
CCTTATTGTCCCTTCCAAAAAAAAGAGAATCAAAATTTTACAAAGAATCAAAGGAATTCTAG
AAAGTATCTGGGCAGAACGCTAGGAGAGATCCAAATTTCCATTGTCTTGCAAGCAAAGCACG
TATTAAATATGATCTGCAGCCATTAAAAAGACACATTCTGTAAATGAGAGAGCCTTATTTTC
CTGTAACCTTCAGCAAATAGCAAAAGACACATTCCAAGGGCCCACTTCTTTACTGTGGGCAT
TTCTTTTTTTTTCTTTTTTTCTTTTTTCCTTTTTTGAGACAAAGTCTCACTCTGTTGCCCAG
GCTAGAATGCAGTGGTGTAATCTCAGCTCACTGCAACCTCTGCTTCCTGGGTTCAAGCGATT
CTCCTGCCTCAGCCTCCCAAGTAACTGGGATTACAGGCGCATGCCACCACGCCTAGCTCATT
TTTGTATTTTTAGTAGAGATGGGATTTTGCCATGTTGGCTAGGCTGGTCTACGAACTCCTGA
CCTCAGGTGATCCACCTGCCTCAGCCTCCCAAAGTGCTGGGATTACAGGCATGAGCCACTAC
ACCCGGCCCCTACTCTGGGCATTTCTTTGATTAAAGAGAAGGGGAGCTCCAACAAGATACAC
CTGCAGCAACTCAGGCCGTCTGATCAGTTCAGGCCAGATCTACACTGCAACCAGCCAGGTCA
GGGGAAAACCAAAGAACCCCACACACCCAATTTACTTAGGCTGATCCAAAATCCATGTATGG
AGAACTCACATGCACCAGGCACTATTTTAGGTGAACTGAATATAAAGAATAGGACCCAGTAC
CTGCATTTACTTAAAGAACTCACAATCTTTTGAGAACATAACTGTTTCATCATGGTTTGGCA
GGAGGCTATGGTACAAGGCACAGCAAGGGTAAGAAGGAGGAAGAAACCAACACCCTACAGAA
ATCAGGGAATGACTCTGAATAGGTGTCACTTAATCTGAGTGTTGGTAATTTGTCAGATAGAC
AAGGGAAAAGGTATTCTAGGTAGAGAGAATACAGTTTGCAAGGCCCAGCCAAGTGAAACAAT
TTGATAAGTTGAGAGAGCAGACGACGATTCAGAATGTTGAAGGGCAAAGGTATTGAGGTGGG
ATGGGTTATGCTGCTATCACAAATAACCCCAAATCTCGGGGGCTTAACAAAGTAAAAGTTTA
GTCTCAGTTGTGCCAGGTCCAATGTAGAACTCTTTGCTCTAGAGACTCTTTAGGGTGGCTTT
CCTTCTAATGGTGACTGTTTGAGACAGTTTGATTTAGTCTTGTGGCTTCAAGGTCACTCTGG
TGATATTTAGCCAGCAGACTGAGGGAACATAGTATGGTATTAGACCCCTCTGTGCTGAAGTG
TCACACATGAGTCCCATTGACTTCTCACTGGCCAGAGCTAGTTACATGCCCCCATCTAGATG
TGCTGAGAAATGTGGCCCCTGGCTGGGAGCCATTTCCCAGAACAACTAACTCTATGCTCTGG
AAGAGGAGCACTAATCTGAGTTGGCCAACAACCATCTCTACCACAGTAGGGTTGGGACTGGT
GGGGCATGAGGCTGGAGTGAAGGTTGGTTTTATCTGCCACGCGTTACAGCTGTGAATTTGTC
TTGAAAGCAACATGGGTCCATTGAAGGGAACCTTGACATCAGTCATGTGGCTGGGACAAGAA
TAGTTACCACTTGCCCGTAATCTCCAACCAGGATTCTCCAGGAGAACCTGAGTTAGACACAT
GGCTTAGGCCTAAACCTACCTGAGTGGTCTTTCTATTTTCCTCCAAATTCAAATCTCAAATC
TTGCTACCCTCTAACTGGCTATGTTGAGAGAGGAAAAAACTTGAAGAGAATGCAGTGTAGCT
TTGGAGTTTTTCACATGCACTTTTCCCAAGATACATAGCAAAATCAATGTCTCCAATTCTAT
TAATGTTGTTAGCAAGTCCTTGTTCCATGCATATTGGTTAATCCATAGCAAATTGCCATTTT
TATAGACTAAATGGTCAAATATTGGCAATTTCATAAGGTTCAGTCTATTACCTACCATGATT
GTATTGGTCACTAACCTGCCTATTTTTAGAATGCTACATATTCATTTGGCTGTTGTTAAATA
GCTATGGATTTTTATAATCAAAACAGGTTGAAAATATGAATCAGTTTAAAACCACATACA.

In the above TTR polynucleotide sequence provided at NCBI Reference Sequence No. NG_009490.1, exons encoding the TTR polypeptide correspond to the union of nucleotides 5137 . . . 5205, 6130 . . . 6260, 8354 . . . 8489, and 11802 . . . 11909, and the intervening sequences correspond to intron sequences. The union of nucleotides 5137 . . . 5205, 6130 . . . 6260, 8354 . . . 8489, and 11802 . . . 11909 corresponds to Consensus Coding Sequence (CCDS) No. 11899.1.

By “transthyretin amyloidosis” is meant a disease associated with an accumulation of amyloid in a tissue of a subject.

By “uracil glycosylase inhibitor” or “UGI” is meant an agent that inhibits the uracil-excision repair system. Base editors comprising a cytidine deaminase convert cytosine to uracil, which is then converted to thymine through DNA replication or repair. In various embodiments, a uracil DNA glycosylase (UGI) prevent base excision repair which changes the U back to a C. In some instances, contacting a cell and/or polynucleotide with a UGI and a base editor prevents base excision repair which changes the U back to a C. An exemplary UGI comprises an amino acid sequence as follows:

>splP14739IUNGI_BPPB2 Uracil-DNA glycosylase
inhibitor
(SEQ ID NO: 231)
MTNLSDIIEKETGKQLVIQESILMLPEEVEEVIGNKPESDILVHTAY
DESTDENVMLLTSDAPEYKPWALVIQDSNGENKIKML.

In some embodiments, the agent inhibiting the uracil-excision repair system is a uracil stabilizing protein (USP). See, e.g., WO 2022015969 A¹, incorporated herein by reference.

As used herein, the term “vector” refers to a means of introducing a nucleic acid molecule into a cell, resulting in a transformed cell. Vectors include plasmids, transposons, phages, viruses, liposomes, lipid nanoparticles, and episomes.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

All terms are intended to be understood as they would be understood by a person skilled in the art. Unless defined otherwise, all technical and scientific terms used herein have the same meaning as commonly understood by one of ordinary skill in the art to which the disclosure pertains

In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended. This wording indicates that specified elements, features, components, and/or method steps are present, but does not exclude the presence of other elements, features, components, and/or method steps. Any embodiments specified as “comprising” a particular component(s) or element(s) are also contemplated as “consisting of” or “consisting essentially of” the particular component(s) or element(s) in some embodiments. It is contemplated that any embodiment discussed in this specification can be implemented with respect to any method or composition of the present disclosure, and vice versa. Furthermore, compositions of the present disclosure can be used to achieve methods of the present disclosure.

The term “about” or “approximately” means within an acceptable error range for the particular value as determined by one of ordinary skill in the art, which will depend in part on how the value is measured or determined, i.e., the limitations of the measurement system.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures.

### BRIEF DESCRIPTION OF THE DRAWINGS

FIGS. 1A, 1B, and 1C. A general schematic of a gene editor complexed with a gRNA targeting a gene of interest. Cas9 protein, guide RNA, Spacer sequence, protospacer sequence, and PAM (protospacer adjacent motif) are identified (FIG. 1A). FIG. 1A discloses SEQ ID NO: 1216. Additionally, schematics of general principles of base editing with cytosine base editors (CBE) (FIG. 1B) and adenine base editors (ABE) (FIG. 1C) are illustrated.

FIG. 2. Alteration of splice donor sites resulting from base editing. Top panel represents normal splicing of RNA transcribed from a gene. Bottom panel represents splicing that may result from transcription of a gene that has a disrupted splice site due to editing.

FIG. 3. Map of the human TTR gene (hTTR gene), shows the location of various restriction enzyme recognition sites, Exons 1-4, and the single guide RNAs GA457, GA459, GA460, and GA461 specified in Table 8.

FIG. 4. Nucleotide sequence of the human TTR gene (UniProtKB-P02766 (TTHY_HUMAN)) from the reference human genome (GRCh38) is shown and depicts the region on the gene where guides GA457, GA459, GA460, and GA461 are located. FIG. 4 discloses SEQ ID NO: 1217.

FIGS. 5A-5C. A schematic showing TTR guides and editing locations for GA457 (FIG. 5A), GA460 (FIG. 5B), and GA461 (FIG. 5C). Human genomic DNA (gDNA) sequences are labeled in black. Guide sequences are highlighted in grey above. Genomic exon sequences are in uppercase letters and intron sequences are in lowercase letters. The main position targeted by ABE editing is labeled with a black arrow. FIGS. 5A, 5B, and 5C disclose SEQ ID NOs: 467, 1218, 469, 1219, 1220, and 1221, respectively, in order of appearance.

FIG. 6. is a graph representing the percent splice editing in human hepatocytes using ABE editing with single guide RNAs GA457, GA459, GA460, and GA461 guide RNAs. The three TTR guide RNAs GA457, GA460, and GA461 show high activity in human hepatocytes. Each of the guides employ the identical tracr sequence and differ only by their RNA spacer sequence which corresponds to specified DNA protospacer sequences on the targeted TTR gene.

FIG. 7 is a flowchart of the ONE-seq protocol for determining candidate off-target sites.

FIG. 8 is a schematic diagram comparison of GA519 and GA457 hybridized to NHP and Human TTR exon 1. FIG. 8 discloses SEQ ID NOs: 467, 1222, 471, and 1223, respectively, in order of appearance.

FIG. 9 is a schematic diagram showing a comparison of GA520 and GA460 hybridized to NHP and Human TTR exon 3. FIG. 9 discloses SEQ ID NOs: 1224-1225 and 1224-1225, respectively, in order of appearance.

FIG. 10 is a bar graph showing hepatic editing of TTR gene by LNP1 and LNP2 in

Non-Human Primates (NHPs) as described in the Examples.

FIG. 11 is a bar graph showing serum TTR protein changes as measured by ELISA in NHP treated with LNP1 and LNP2 as described in the Examples.

FIG. 12 is a bar graph showing serum TTR protein changes as measured by mass spectrometry in NHP treated with LNP1 and LNP2 as described in the Examples.

FIGS. 13A and 13B are a bar graphs showing serum Alanine Aminotransferase (ALT), FIG. 13A, and serum Aspartate Aminotransferase (AST), FIG. 13B, concentrations in NHP treated with LNP1 and LNP2 as described in the Examples.

FIGS. 14A and 14B are a bar graphs showing serum Lactate Dehydrogenase (LDH), FIG. 14A, and serum Glutamate Dehydrogenase (GDH), FIG. 14B, concentrations in NHP treated with LNP1 and LNP2 as described in the Examples.

FIGS. 15A and 15B are a bar graphs showing serum Gamma-Glutamyl Transferase (GGT), FIG. 15A, and serum Alkaline Phosphatase (AP), FIG. 15B, concentrations in NHP treated with LNP1 and LNP2 as described in the Examples.

FIG. 16 is a bar graph showing serum total bilirubin concentrations in NHP treated with LNP1 and LNP2 as described in the Examples.

FIG. 17 is a bar graph showing serum creatine kinase concentrations in NHP treated with LNP1 and LNP2 as described in the examples.

FIG. 18 shows bar graphs of serum cytokine concentrations (MCP-1, upper left panel; IL-6, upper right panel; IP-10, lower left panel; and IL-IRA, lower right panel) over time in NHP treated with LNP1 and LNP2 as described in the Examples.

FIGS. 19A and 19B are plots of plasma pharmacokinetic profiles of iLipid (FIG. 19A) and PEG lipids (FIG. 19B) in NHP treated with LNP1 and LNP2 as described in the Examples.

FIG. 20 is a bar graph showing hepatic editing of TTR gene by LNP3 in NHPs as described in the Examples.

FIG. 21 is a plot showing serum TTR protein changes measured by ELISA in NHP treated with LNP3 as described in the Examples.

FIG. 22 is a plot showing serum TTR protein changes measured by liquid chromatography-mass spectrometry in NHP treated with LNP3 as described in the Examples.

FIGS. 23A and 23B are a bar graphs showing serum Alanine Aminotransferase (ALT), FIG. 23A, and serum Aspartate Aminotransferase (AST), FIG. 23B, concentrations in NHP treated with LNP3 as described in the Examples.

FIGS. 24A and 24B are a bar graphs showing serum Lactate Dehydrogenase (LDH), FIG. 24A, and serum Glutamate Dehydrogenase (GDH), FIG. 24B, concentrations in NHP treated with LNP3 as described in the Examples.

FIGS. 25A and 25B are a bar graphs showing serum Gamma-Glutamyl Transferase (GGT), FIG. 25A, and serum Alkaline Phosphatase (AP), FIG. 25B, concentrations in NHP treated with LNP3 as described in the Examples.

FIG. 26 is a bar graph showing serum total bilirubin concentrations in NHP treated with LNP2 as described in the Examples.

FIG. 27 is a bar graph showing serum creatine kinase concentrations in NHP treated with LNP3 as described in the examples.

FIGS. 28A and 28B are plots of plasma pharmacokinetic profiles of iLipid (FIG. 28A) and PEG lipids (FIG. 28B) in NHP treated with LNP1 and LNP2 as described in the Examples.

FIGS. 29A-29C are plots showing base editing efficiency for base editor systems comprising the indicated base editors in combination with the indicated guide RNAs targeting a transthyretin (TTR) polynucleotide. FIG. 29A is a plot of A→G base editing efficiencies at a conserved splice site motif using the indicated base editors and guides. FIG. 29B is a plot of C>T base editing efficiencies in a splice site motif using the indicated base editors and guides. FIG. 29C is a plot of indel editing efficiencies.

FIG. 30 is a plot showing editing efficiency for a bhCas12b endonuclease used in combination with the indicated guide RNAs targeting a transthyretin (TTR) polynucleotide.

FIG. 31 provides a bar graph showing human TTR protein concentrations measured by ELISA in PXB-cell hepatocytes prior to transfection. Each condition was run in triplicate, as represented by each dot in the assay. Bar graphs illustrate the mean TTR protein concentrations and error bars indicate the standard deviation.

FIG. 32 provides a combined bar graph and plot showing editing rates in PXB-cell hepatocytes at the targeted site assessed at 13 days post-transfection by NGS(squares, right axis), and human TTR protein concentrations assessed 7 days post-transfection by ELISA (bars, left axis). Each condition was run in triplicate, as represented by each dot. In FIG. 32, the dotted line indicates the average human TTR concentration in cells edited using the base editing system ABE8.8_sgRNA_088. The starred sample (Cas9_gRNA991*) indicates that maximum indel rate within the protospacer region was measured, rather than rate of target base-editing.

FIG. 33 provides a combined bar graph and plot showing Editing rates in PXB-cell hepatocytes at the targeted site assessed at 13 days post-transfection by NGS(squares, right axis), and human TTR protein concentrations assessed 13 days post-transfection by ELISA (bars, left axis). Each condition was run in triplicate, as represented by each dot. In FIG. 33. The dotted line indicates the average human TTR concentration in cells edited using the base editing system ABE8.8_sgRNA_088. Starred sample indicates that maximum indel rate within the protospacer region was measured, rather than rate of target base-editing.

FIG. 34 provides a bar graph showing cyno TTR protein concentrations measured by ELISA in primary cyno hepatocyte co-culture supernatants prior to transfection. Each condition was run in triplicate, as represented by each dot in the assay. The bars illustrate the mean TTR protein concentrations and error bars indicate the standard deviation.

FIG. 35 provides a combined bar graph and plot showing editing rates in primary cyno hepatocyte co-cultures at the targeted site assessed at 13 days post-transfection by NGS (squares, right axis), and cyno TTR protein concentrations assessed 7 days post-transfection by ELISA (bars, left axis). Each condition was run in triplicate, as represented by each dot in the graph. The dotted line indicates the average cyno TTR concentration in cells edited using a base editing system including ABE8.8_sgRNA_088.

FIG. 36 provides a combined bar graph and plot showing editing rates in primary cyno hepatocyte co-cultures at the targeted site assessed at 13 days post-transfection by NGS (squares, right axis), and cyno TTR protein concentrations assessed 13 days post-transfection by ELISA (bars, left axis). Each condition was run in triplicate, as represented by each dot in the graph. The dotted line indicates the average cyno TTR concentration in cells edited using the base editing system ABE8.8_sgRNA_088.

FIGS. 37A and 37B present schematics showing the TTR promoter sequence aligned to gRNAs designed for a screen. In FIG. 37A, The gRNAs are shown above or below the sequence shown in the figure depending on their strand orientation. In each of FIGS. 37A and 37B, the gRNA protospacer sequence plus PAM sequence is shown in each annotation. The nucleotide sequence shown in FIGS. 37A and 37B is provided in the sequence listing as SEQ ID NO: 1229 and the amino acid sequence shown in FIGS. 37A and 37B is provided in the sequence listing as SEQ ID NO: 1230.

FIG. 38 provides a bar graph showing next-generation sequencing (NGS) data from three replicates of HepG2 cells transfected with mRNA encoding the indicated editor (indicated above the bars) and gRNA encoding the indicated gRNA (indicated along the x-axis). Dots represent individual data points for each edit type (i.e., indel, max. A-to-G, max. C-to-T) shown. Max A-to-G or max. C-to-T reflects the highest editing frequency for any A or C base within the gRNA protospacer. Three replicates were performed on the same day.

FIG. 39 provides a bar graph showing TTR knockdown data. Individual data points for 2 replicates of TTR expression data are plotted. Three technical replicates for each data point for the RT-qPCR were performed and the mean is plotted for 2 biological data points. All data are from transfections were performed on the same day. RT-qPCR analysis was performed relative to untreated controls in the same RT-qPCR plate as the test well. ACTB was used as an internal control for each sample. Untreated cells had a different TTR: ACTB ratio than transfected cells, which led to artificially reduced relative TTR expression (0.30-0.42) in cells transfected with negative control catalytically dead Cas9 editor or gRNA that would not affect TTR expression.

FIGS. 40A and 40B provide schematics showing the location of promoter tiling gRNAs effective in a TTR RT-qPCR knockdown assay. All gRNAs that demonstrated comparable or improved TTR knockdown as compared with a nuclease approach are shown. Five highly effective gRNAs, as measured by TTR RT-qPCR, were gRNA1756 ABE, gRNA1764 ABE, gRNA1790 CBE, gRNA1786 ABE, and gRNA1772 ABE. A few gRNAs that lowered TTR transcript levels overlapped with putative functional elements including a putative TATA box (transcription initiation site) and a start codon (translation initiation site) as indicated in FIGS. 40A and 40B. In FIGS. 40A and 40B,*indicates the gRNA was highly effective when paired with either an ABE or CBE;**indicates editing frequency was <50% for this gRNA, not intending to be bound by theory, this could indicate that the gRNA was acting though a mechanism distinct from or in addition to base editing; and***indicates both that the gRNA was highly effective when paired with either an ABE or CBE and that editing frequency was <50% for this gRNA. In FIG. 40B, five potent gRNA's, as measure dby TTR RT-qPCR, are shown in white (gRNA1756 ABE, gRNA1764 ABE, gRNA1790 CBE, gRNA1786 ABE, and gRNA1772 ABE). The nucleotide sequence shown in FIG. 40A is provided in the sequence listing as SEQ ID NO: 1226 and the amino acid sequence shown in FIG. 40A is provided in the sequence listing as SEQ ID NO: 1227. The nucleotide sequence shown in FIG. 40B corresponds to SEQ ID NO: 1228.

FIG. 41 provides a bar graph showing editing rates at the targeted sites assessed at 72 hours post-transfection by NGS. Each experimental condition was run in triplicate and is displayed as an average with standard error of the mean. Total splice site disruption without unintended in-gene edits is shown as the left bar of each pair of bars, and unintended edits are shown as the right bar of each pair of bars. The total editing by the gRNA991 spCas9 control is displayed as the left bar for the “gRNA991+spCas9” sample.

FIG. 42 is a graph that shows percent base editing in primary human hepatocytes at various doses total RNA (ng/TA/ml) where GA521 was provided as the guide RNA. GA521 showed sustained base editing of greater than 40% in primary human hepatocytes.

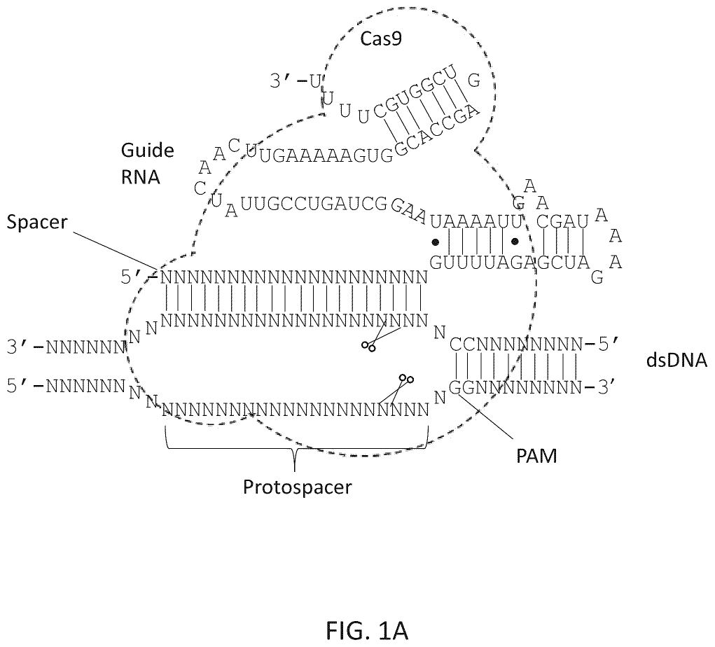

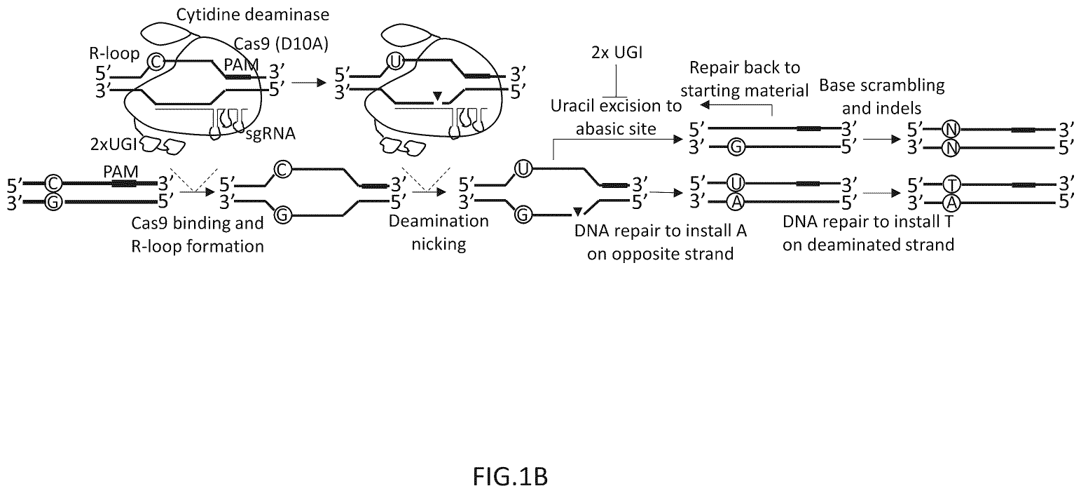

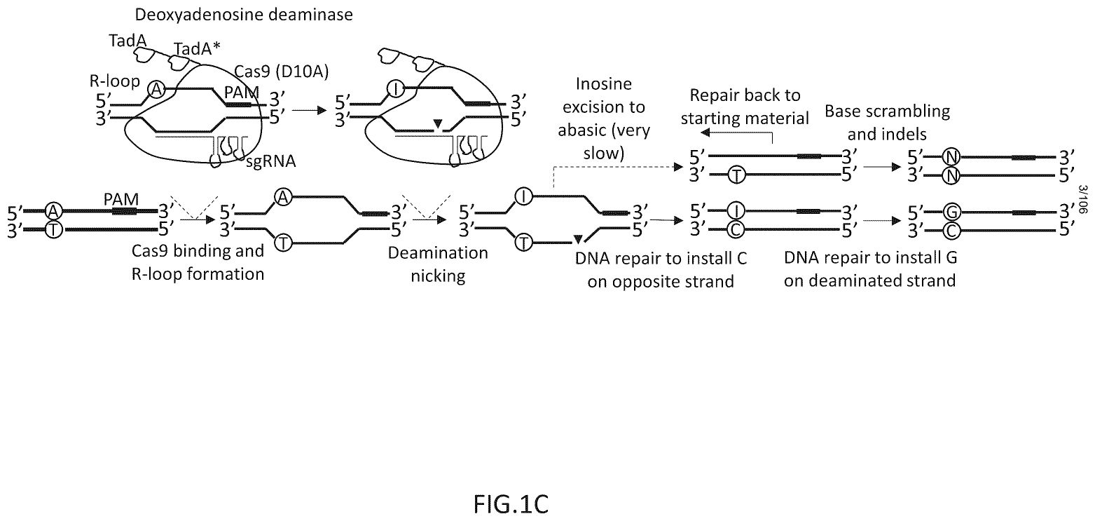

### DETAILED DESCRIPTION

Provided herein are compositions for gene modification or editing and methods of using the same to treat or prevent conditions associated with the extracellular deposition in various tissues of amyloid fibrils formed by the aggregation of misfolded transthyretin (TTR) proteins. Such conditions include, but are not limited to, polyneuropathy due to hereditary transthyretin amyloidosis (hATTR-PN) and hereditary cardiomyopathy due to transthyretin amyloidosis (hATTR-CM), both associated with autosomal dominant mutations of the TTR gene, and an age-related cardiomyopathy associated with wild-type TTR proteins (ATTRwt), also known as senile cardiac amyloidosis. Compositions and methods directed to editing the TTR gene using an editing system such as one comprising a base editor and guide RNAs are disclosed. The invention is based, at least in part, on the discovery that editing can be used to disrupt expression of a transthyretin polypeptide or to edit a pathogenic mutation in a transthyretin polypeptide. In one particular embodiment, the invention provides guide RNA sequences that are effective for use in conjunction with a base editing system for editing a transthyretin (TTR) gene sequence to disrupt splicing or correct a pathogenic mutation. In another embodiment, the invention provides guide RNA sequences that target a Cas12b nuclease to edit a TTR gene sequence, thereby disrupting TTR polypeptide expression.

Accordingly, the invention provides guide RNA sequences suitable for use with ABE and/or BE4 for transthyretin (TTR) gene splice site disruption and guide RNA sequences suitable for use with bhCas12b nucleases for disruption of the transthyretin (TTR) gene. In embodiments, the compositions and methods of the present invention can be used for editing a TTR gene in a hepatocyte. The methods provided herein can include reducing or eliminating expression of TTR in a hepatocyte cell to treat an amyloidosis.

### Transthyretin Protein and Gene

Transthyretin (TTR), originally known as prealbumin, is a 55-kDa transport protein for both thyroxine (T4) and retinol-binding protein, that circulates in soluble form in the serum and cerebrospinal fluid (CSF) of healthy humans. TTR is understood to be primarily synthesized in the liver. Under normal conditions, TTR circulates as a homotetramer with a central channel. The wild-type TTR monomer is 147 amino acids in length and has the amino acid sequence below:

(SEQ ID NO: 464)
MASHRLLLLC LAGLVFVSEA GPTGTGESKC PLMVKVLDAV
RGSPAINVAV HVFRKAADDT WEPFASGKTS ESGELHGLTT
EEEFVEGIYK VEIDTKSYWK ALGISPFHEH AEVVFTANDS
GPRRYTIAAL LSPYSYSTTA VVTNPKE.

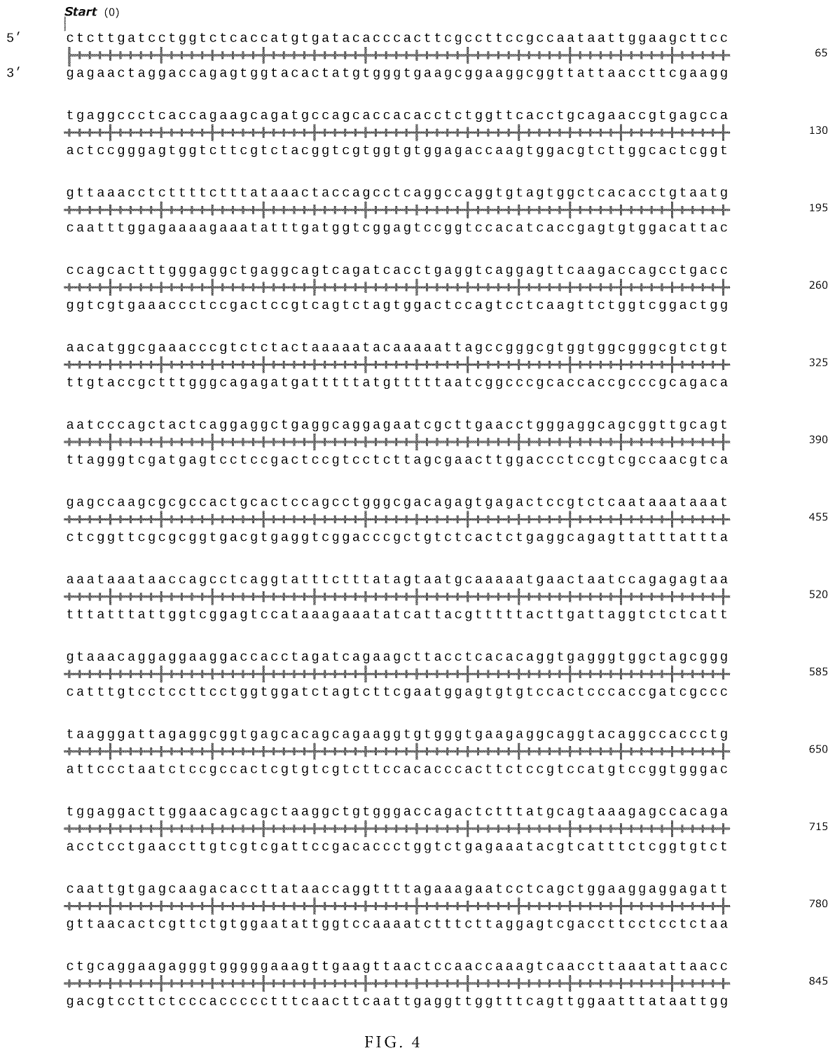

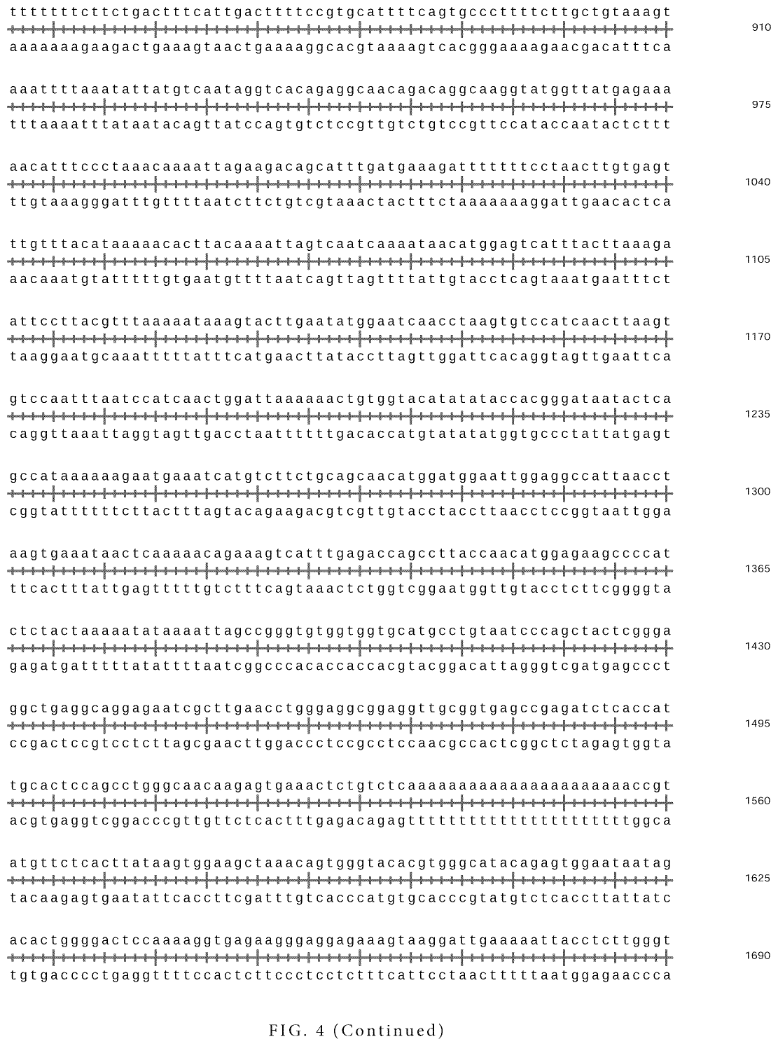

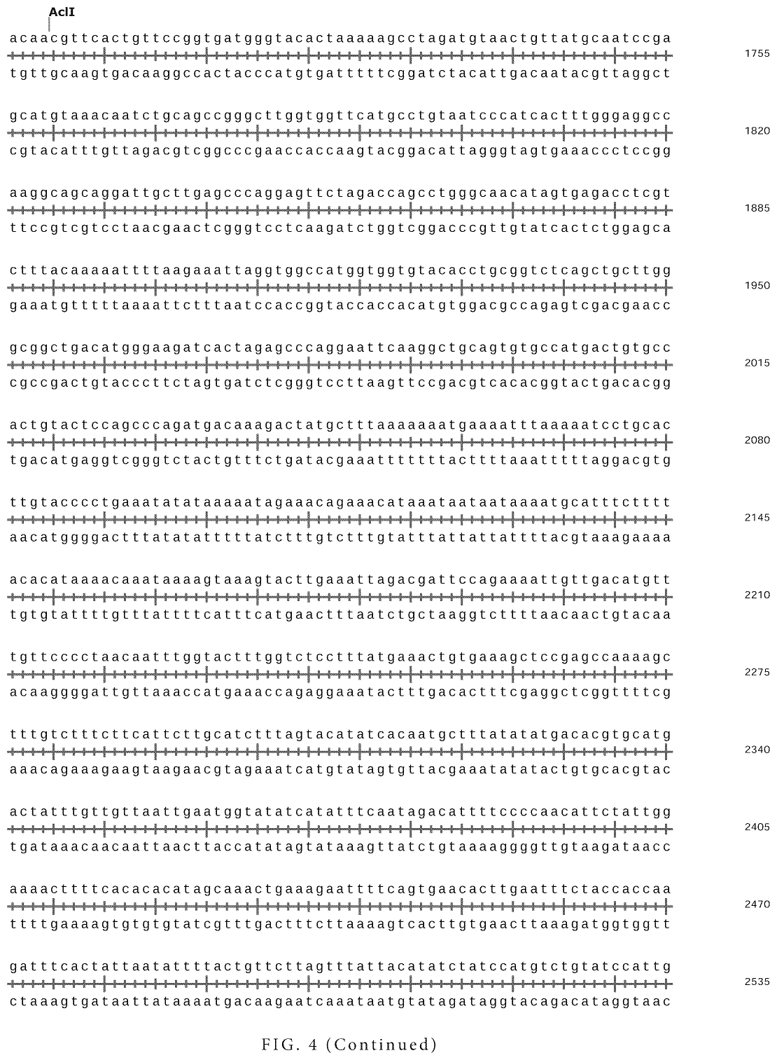

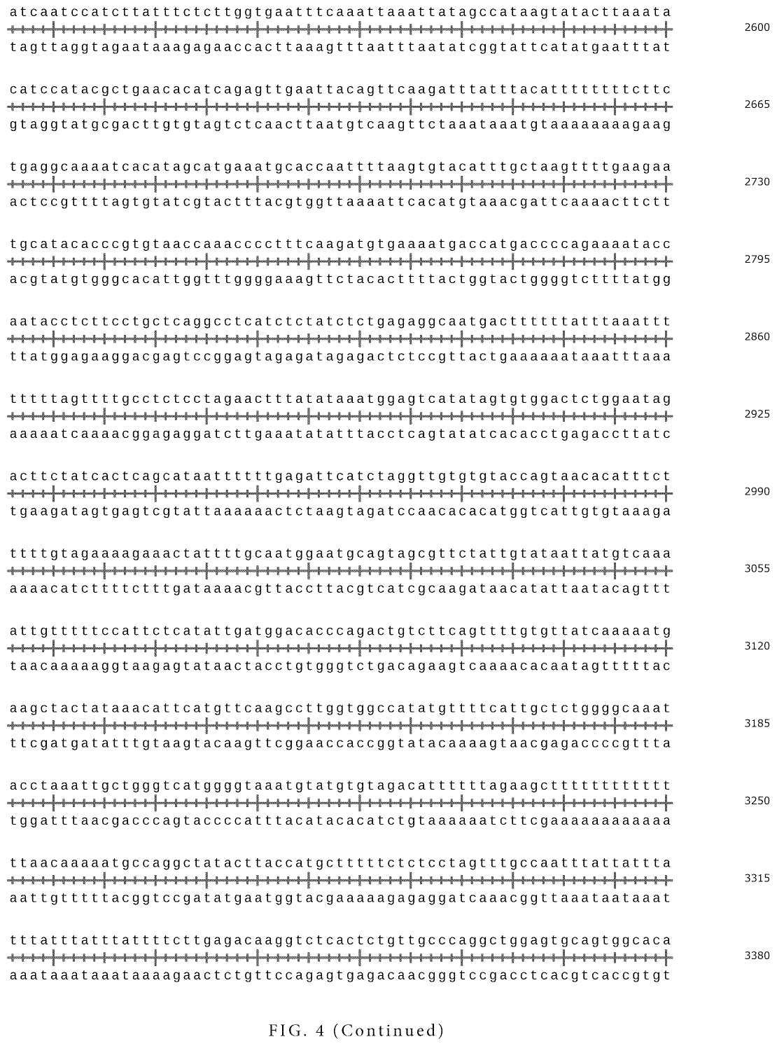

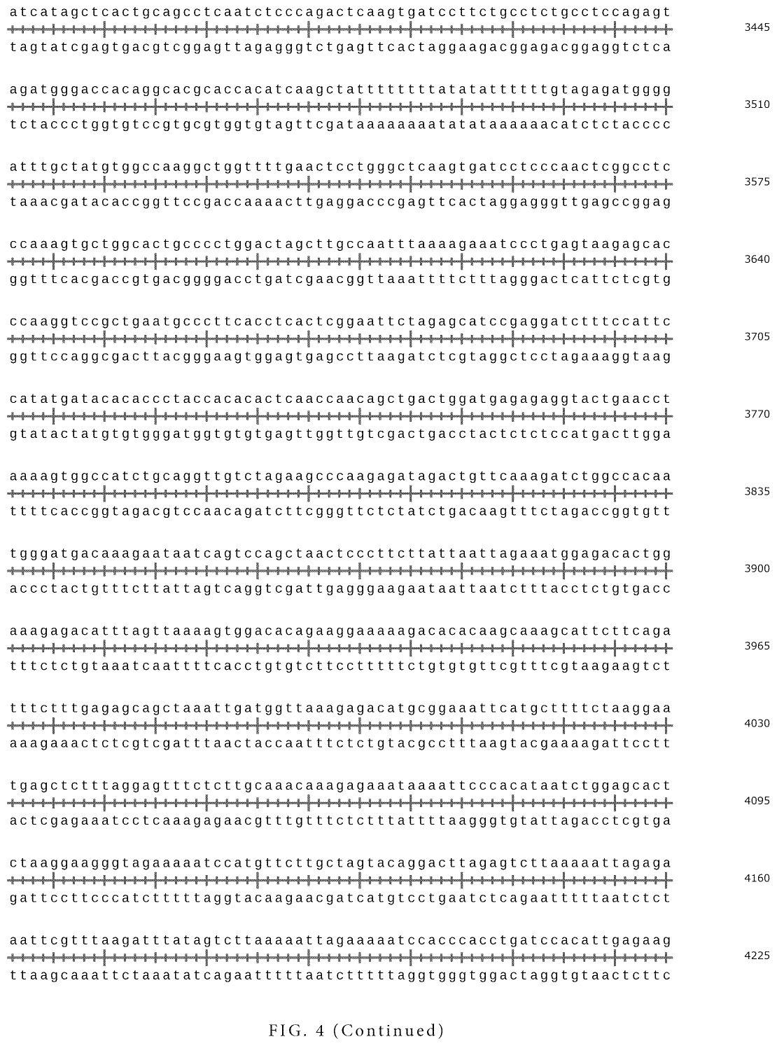

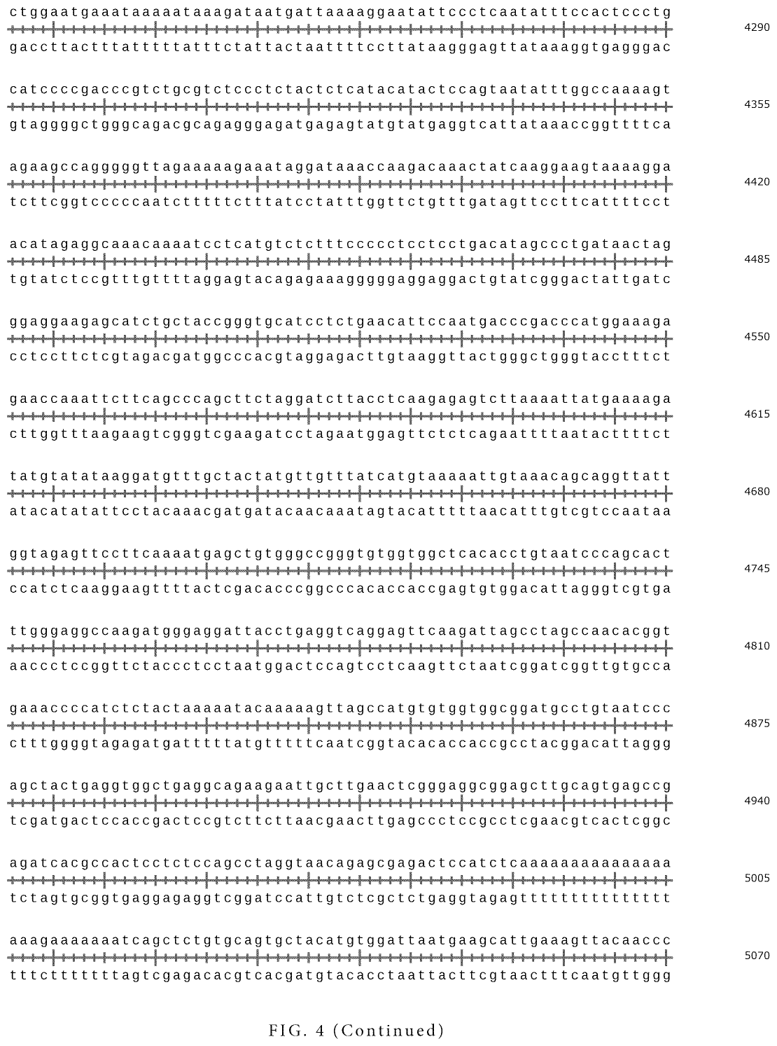

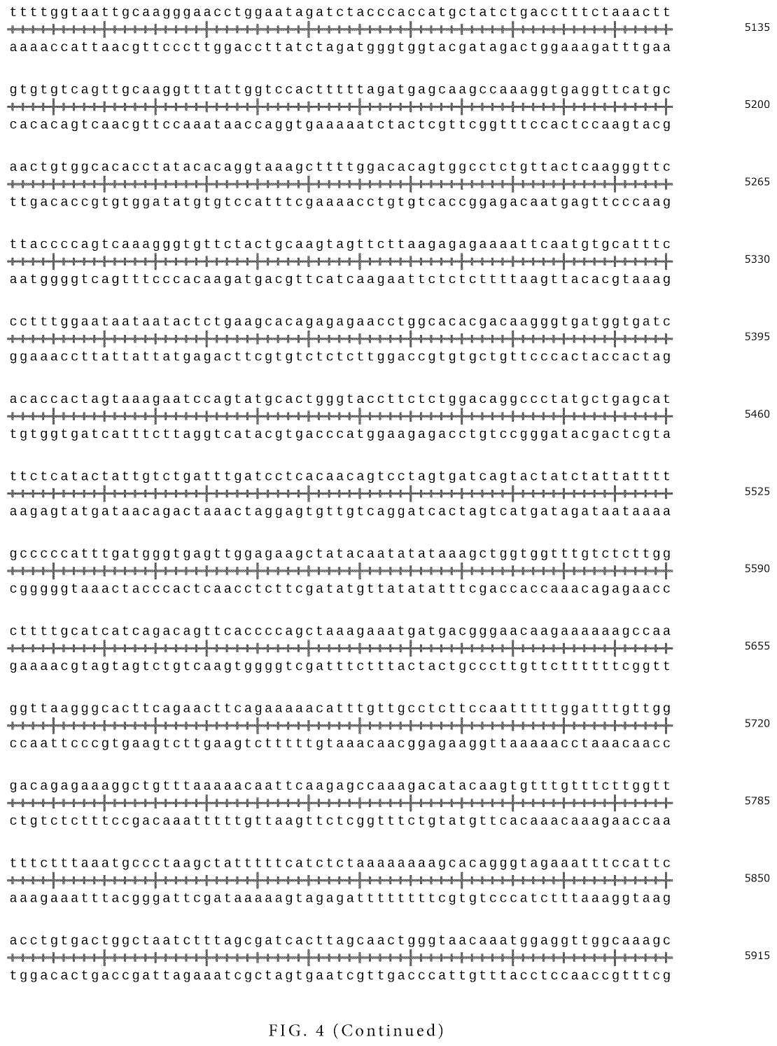

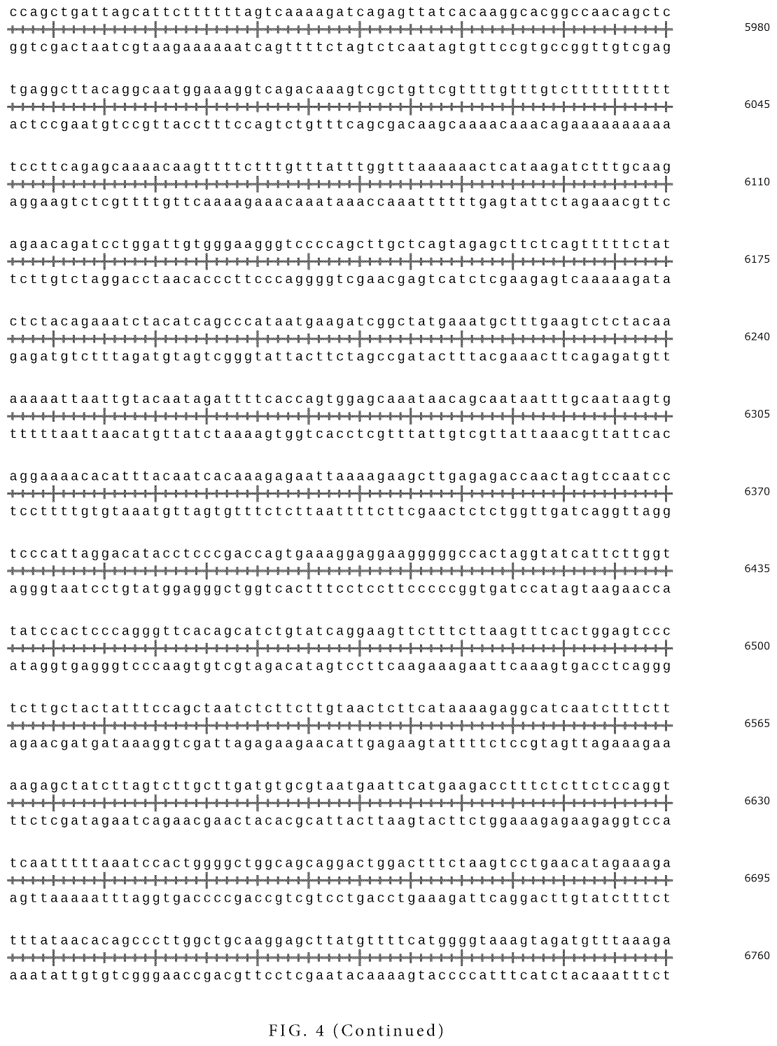

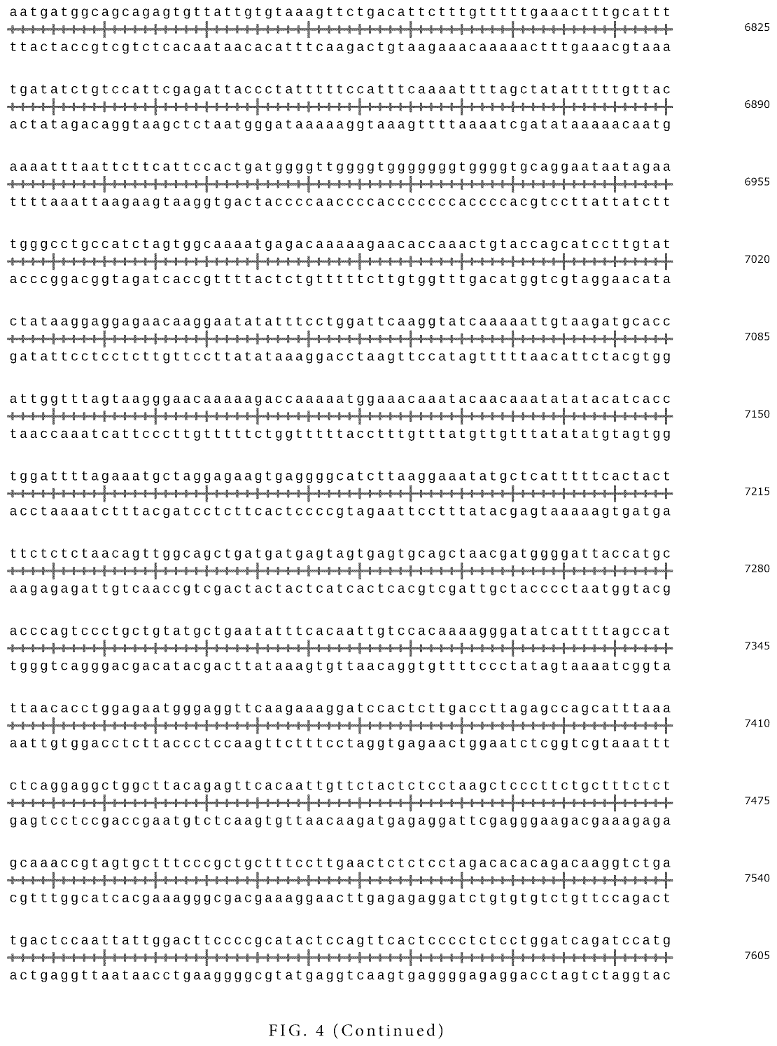

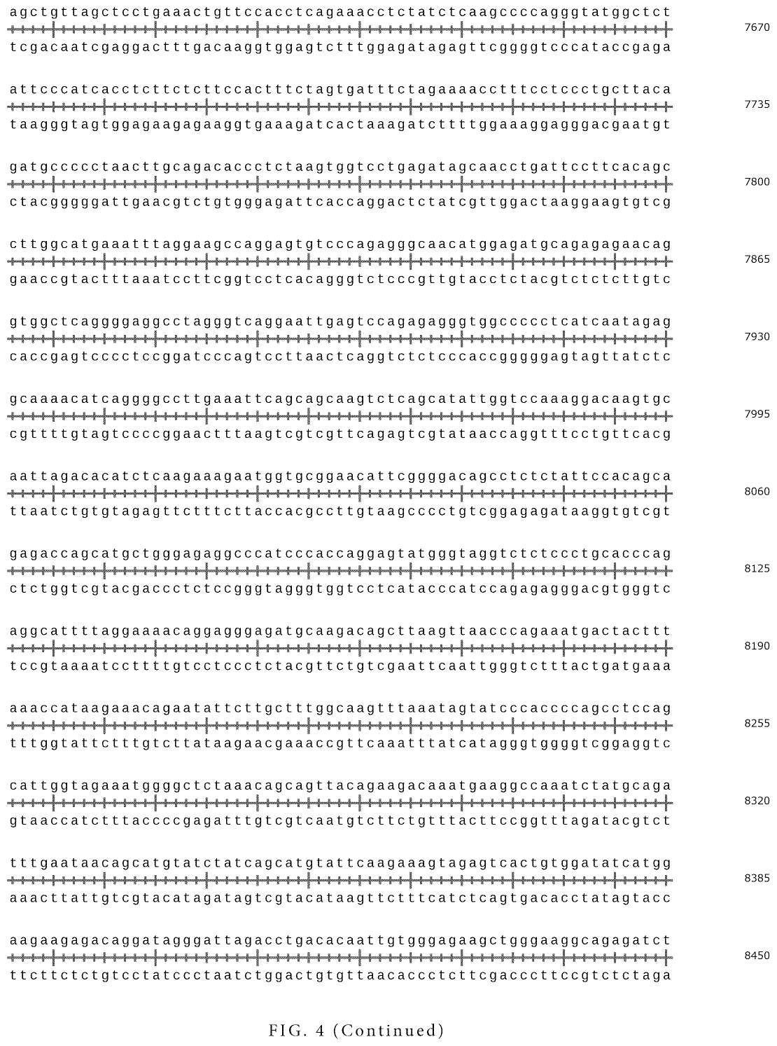

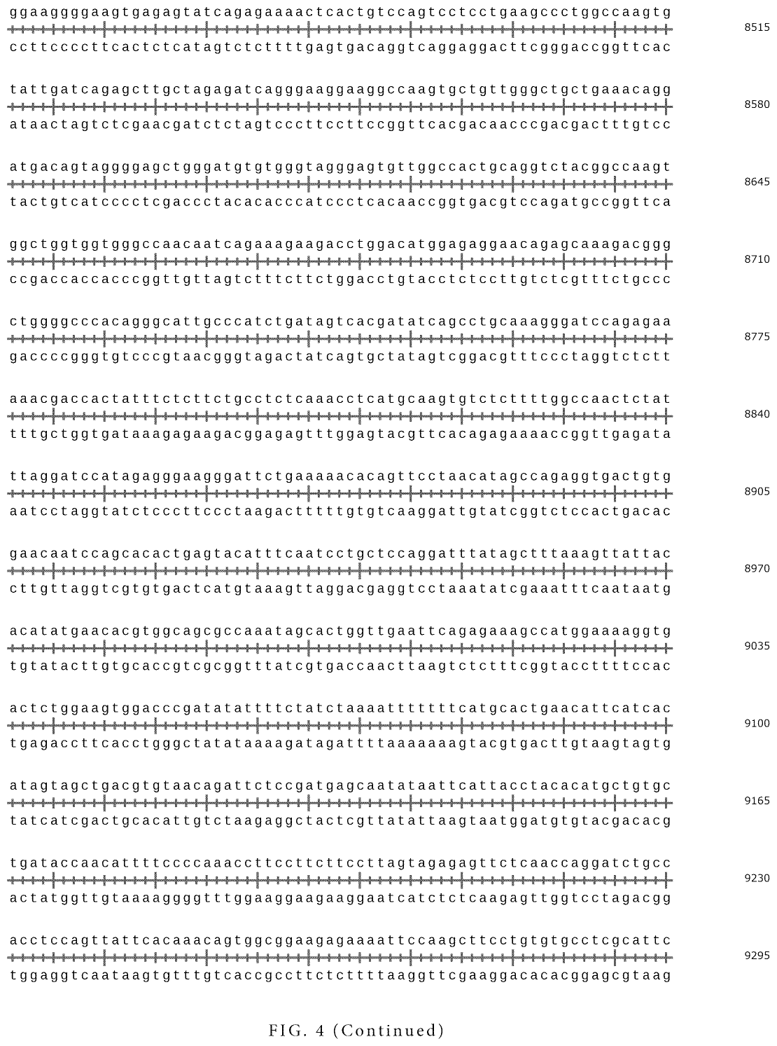

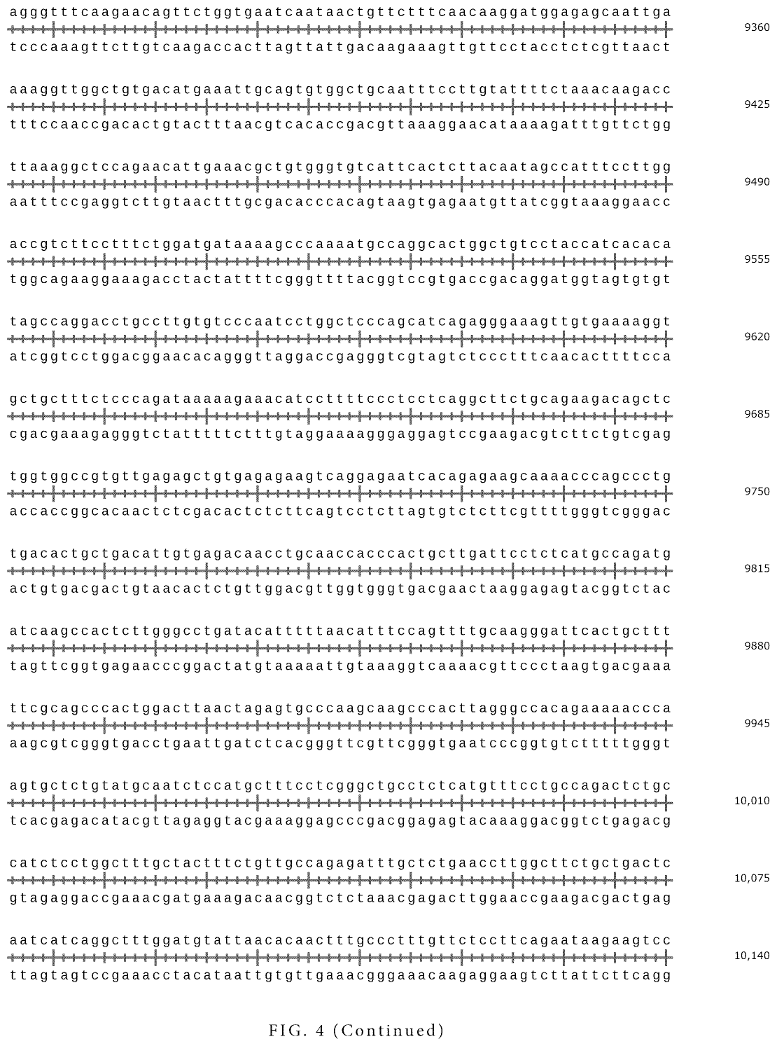

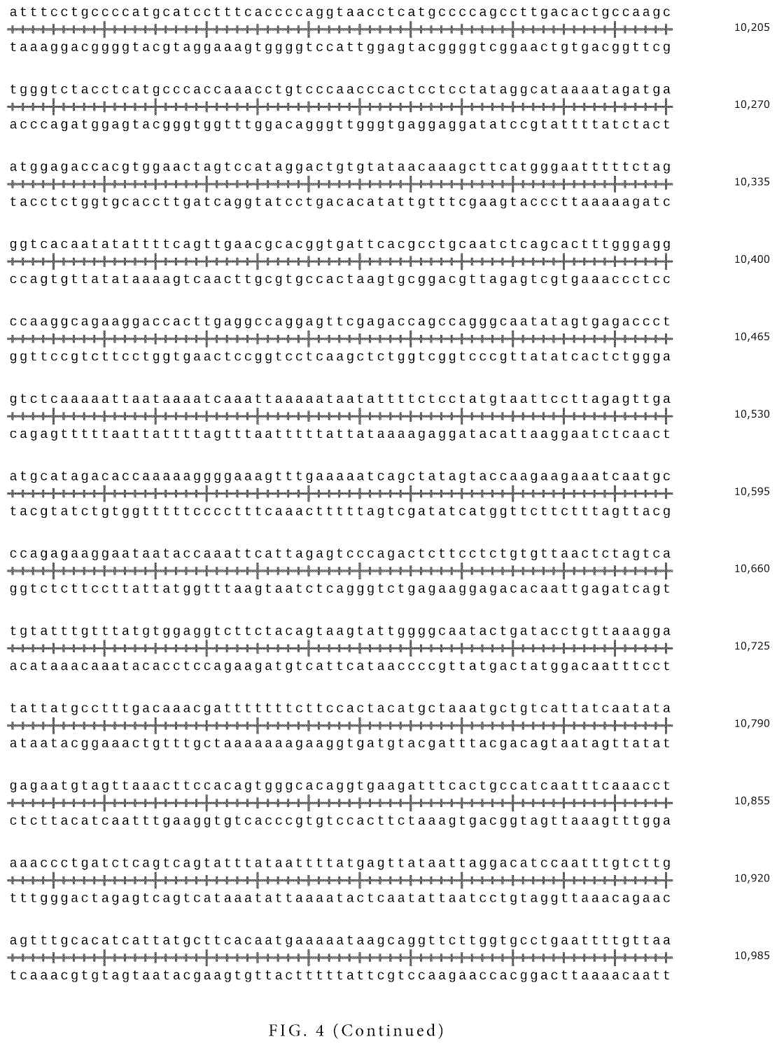

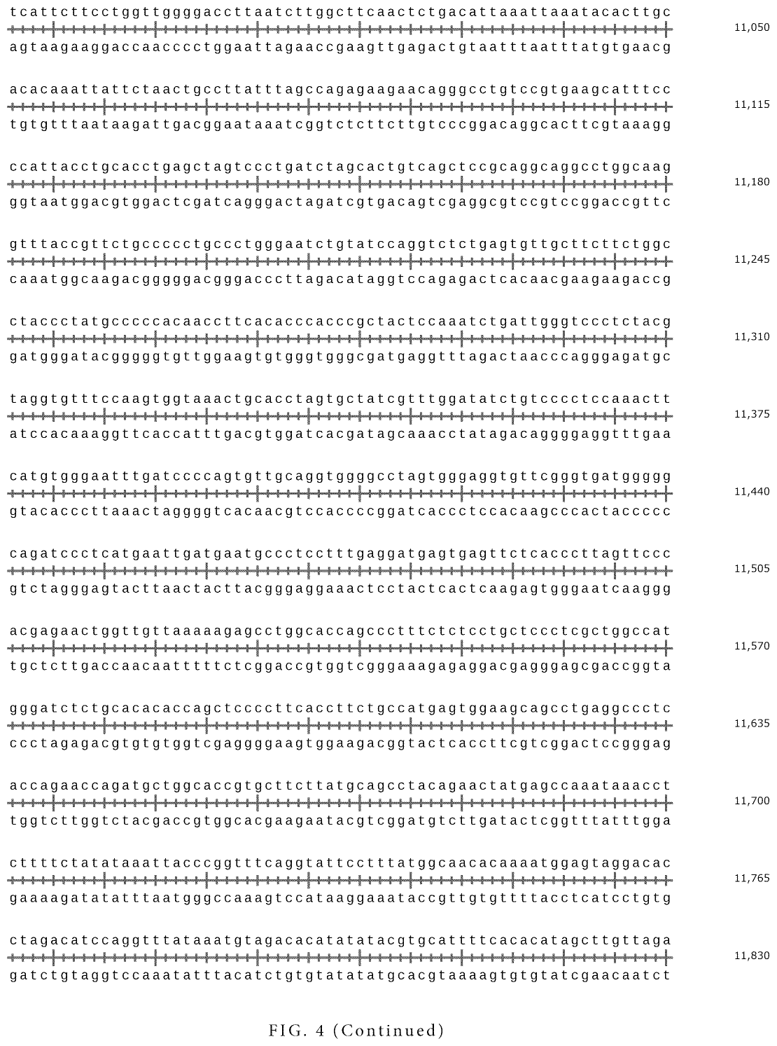

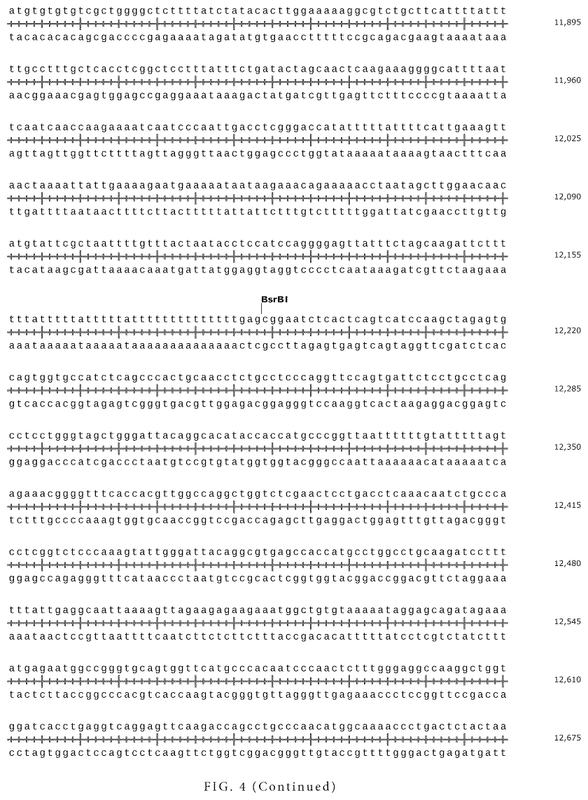

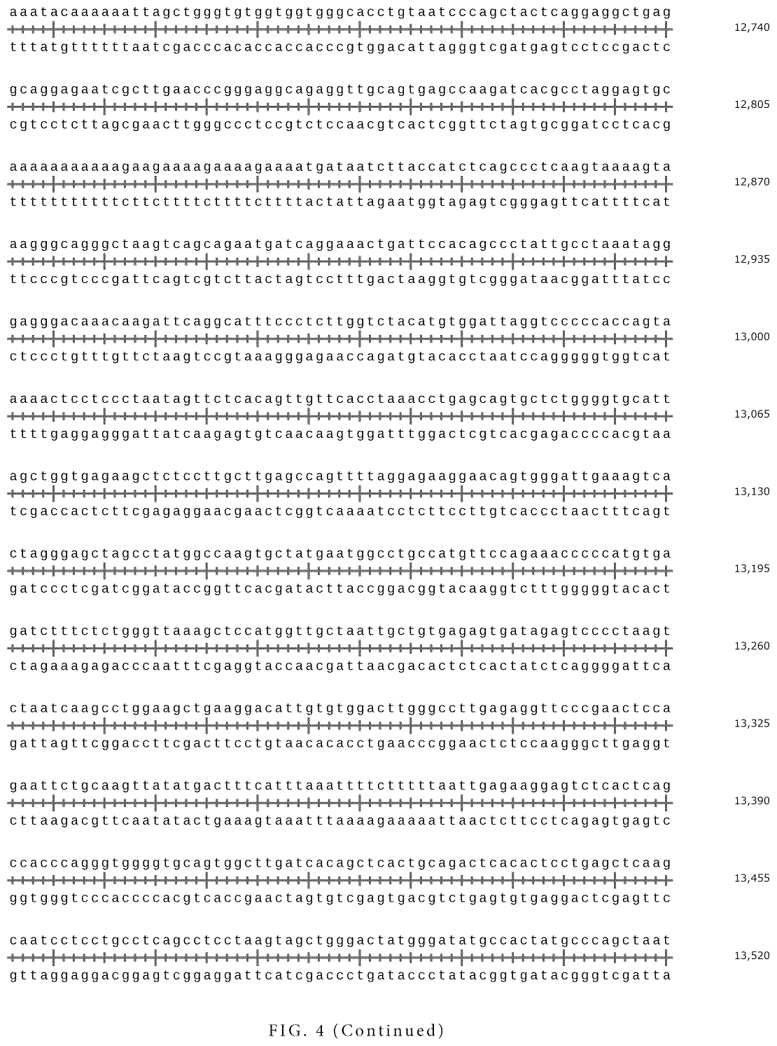

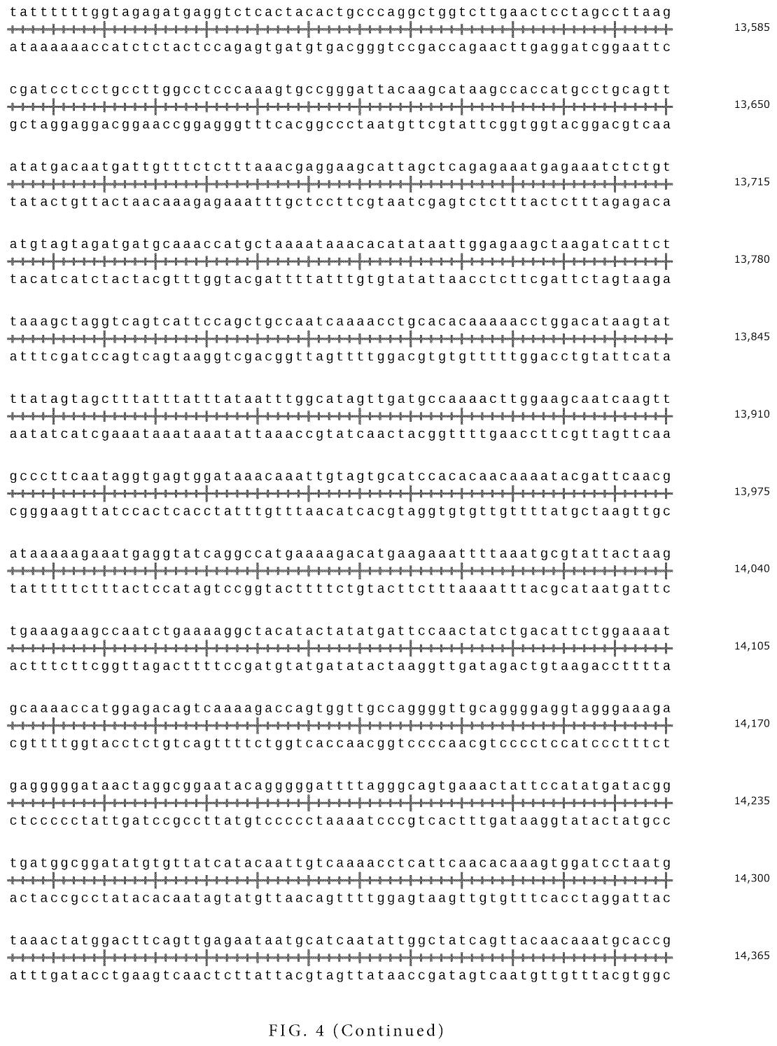

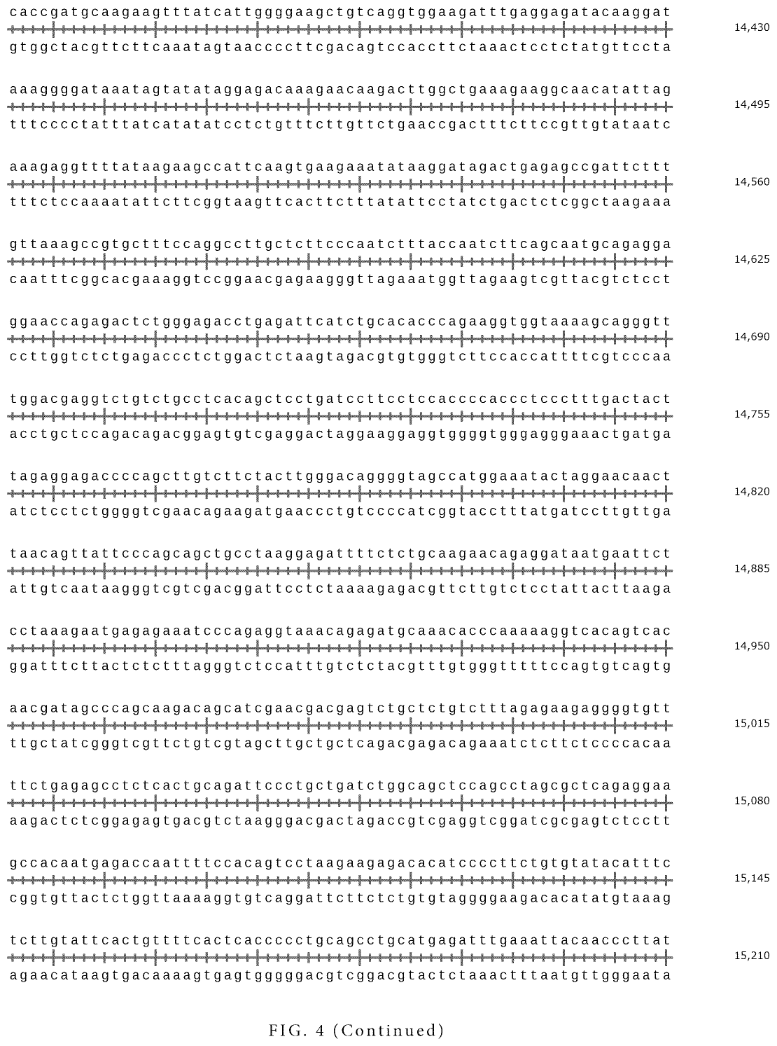

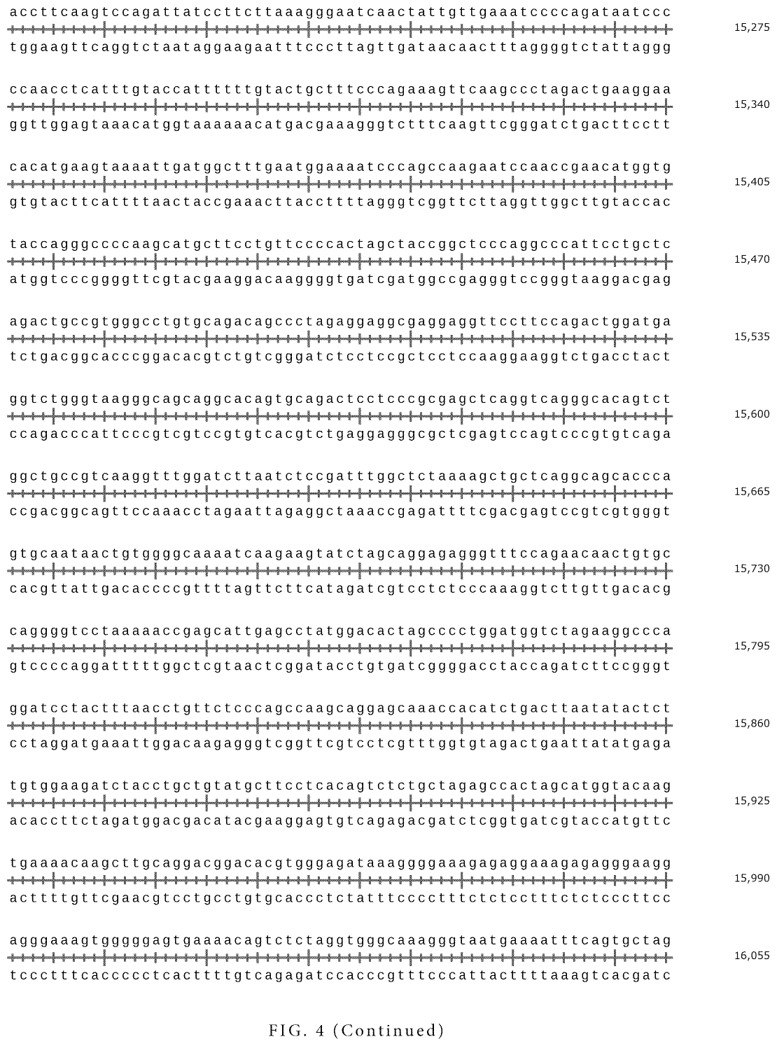

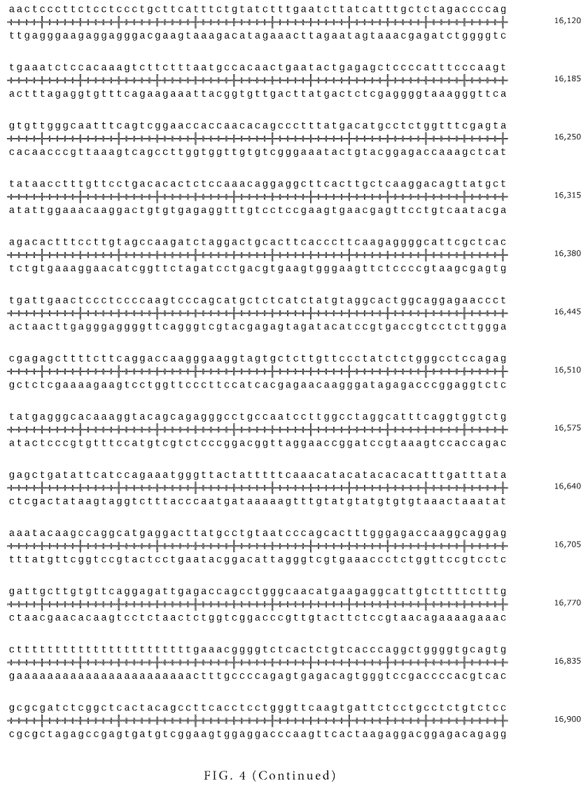

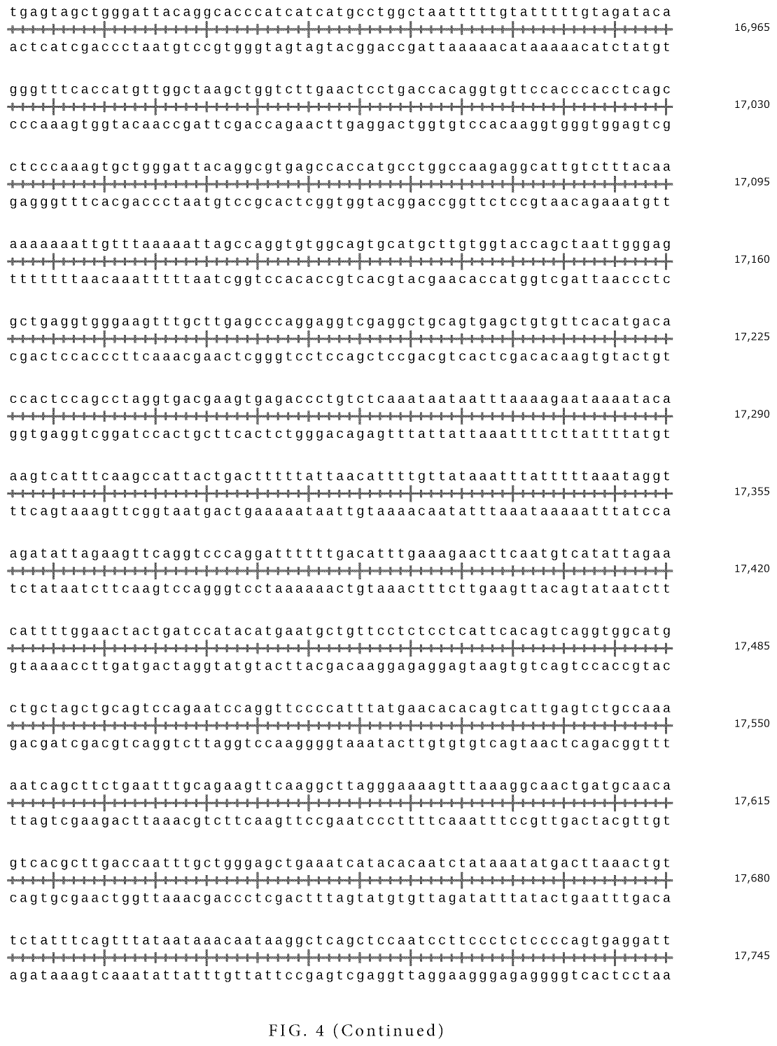

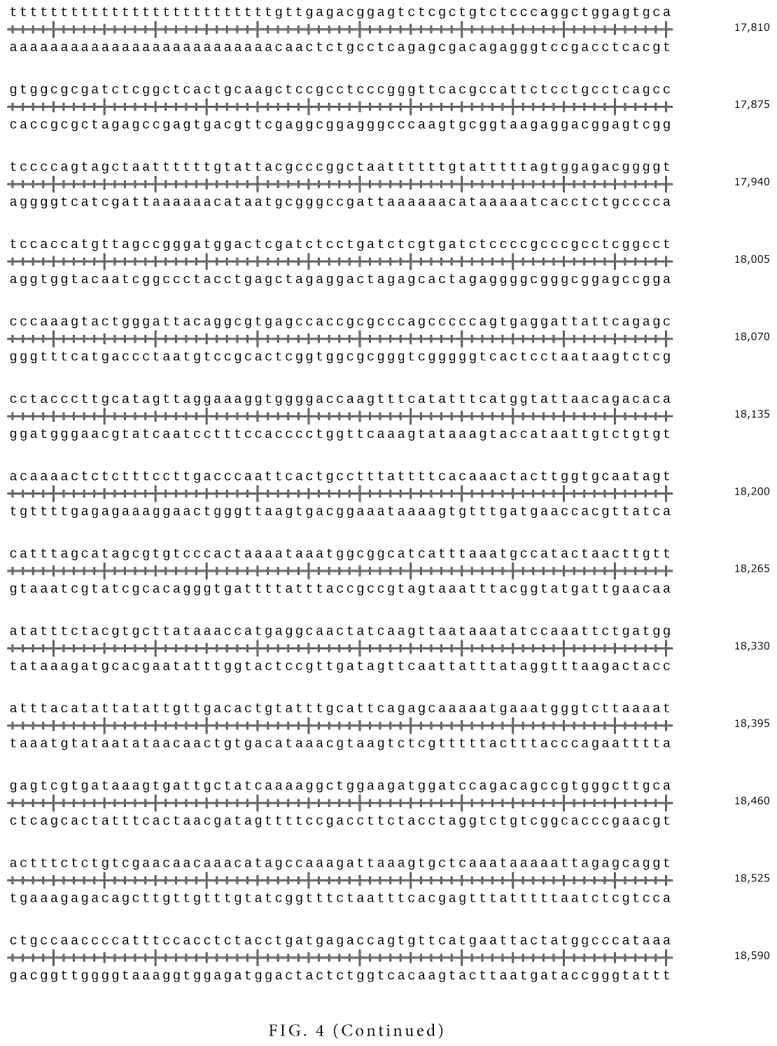

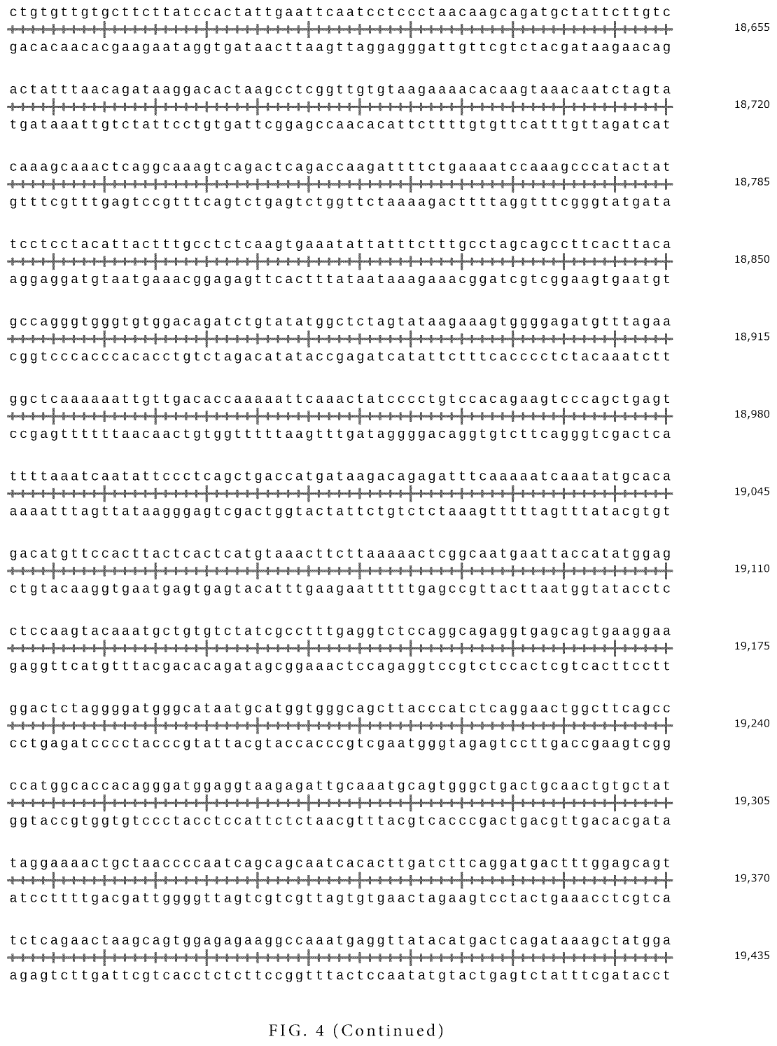

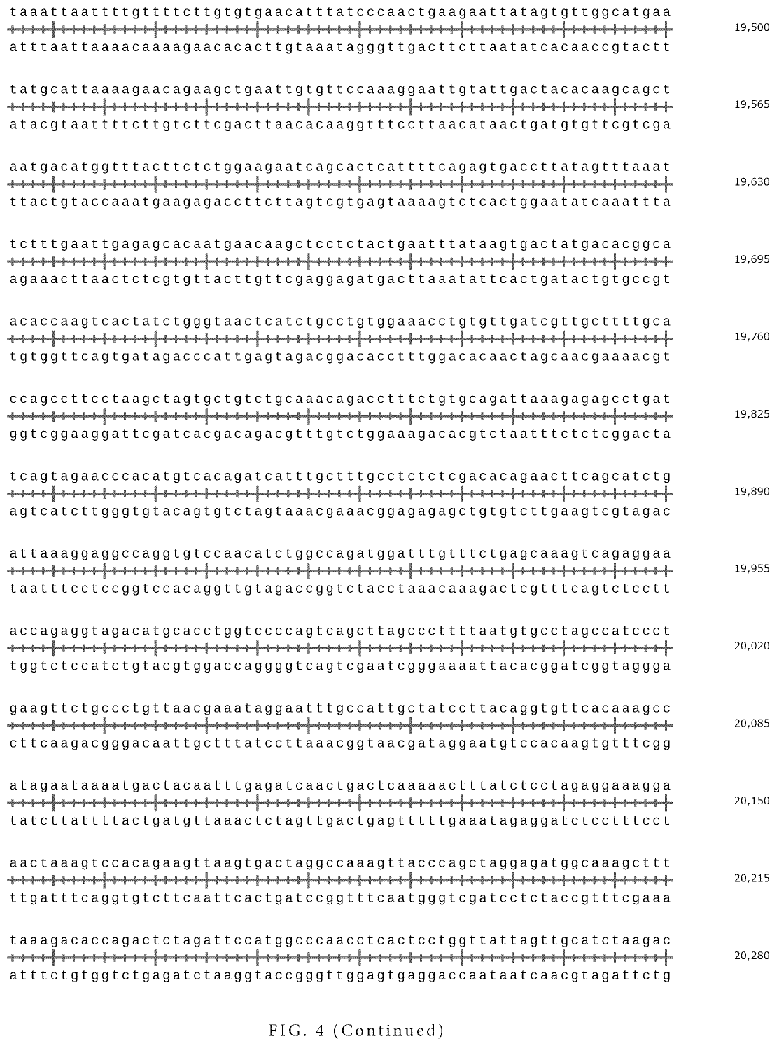

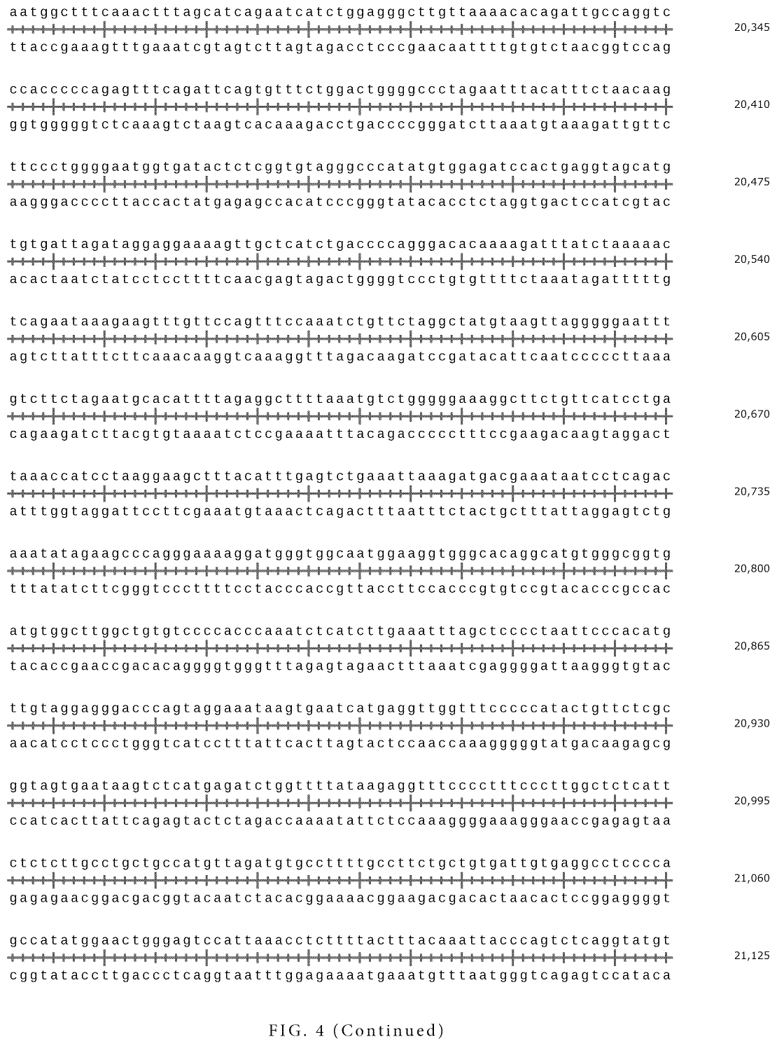

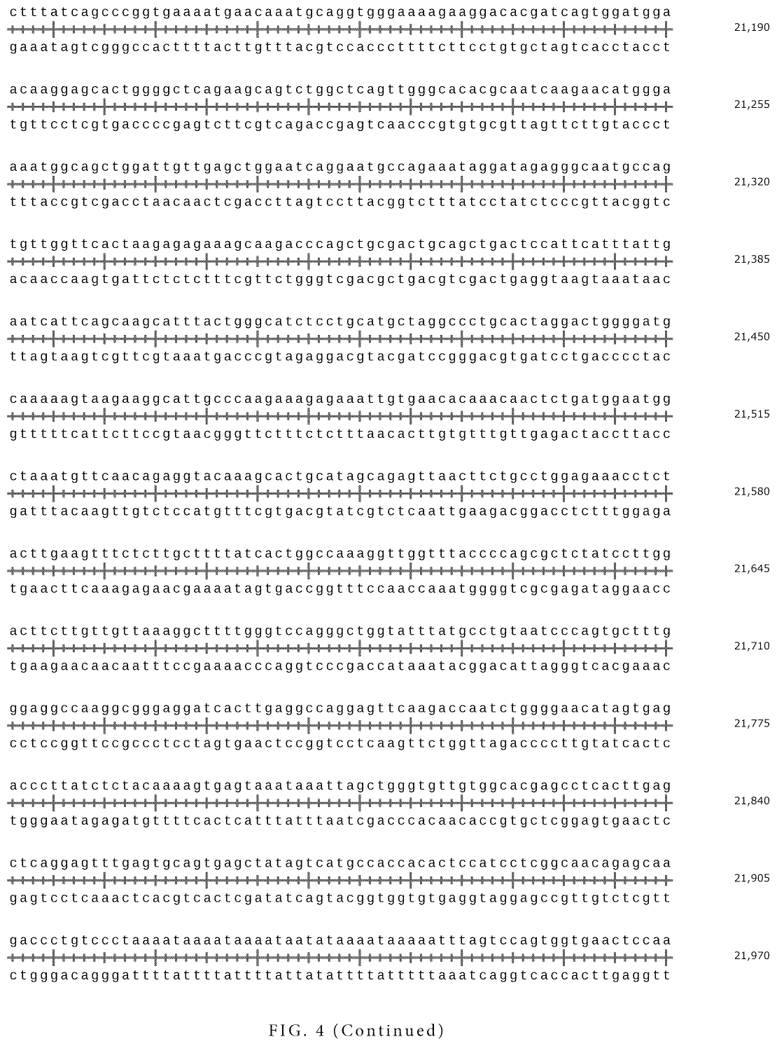

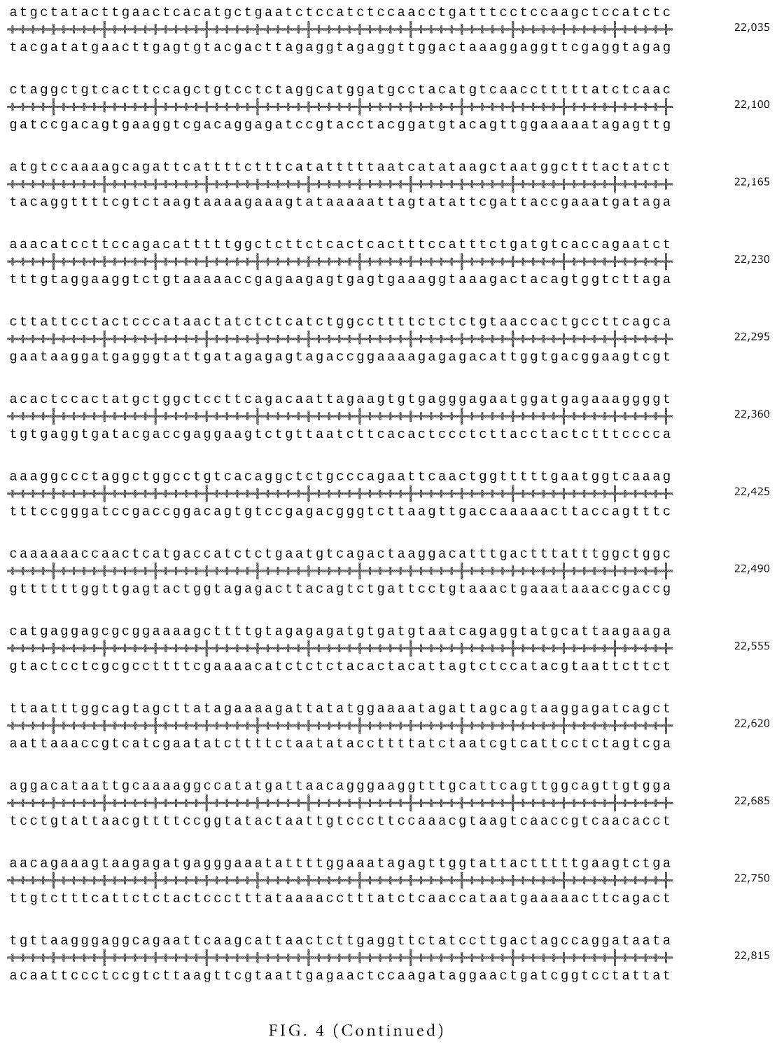

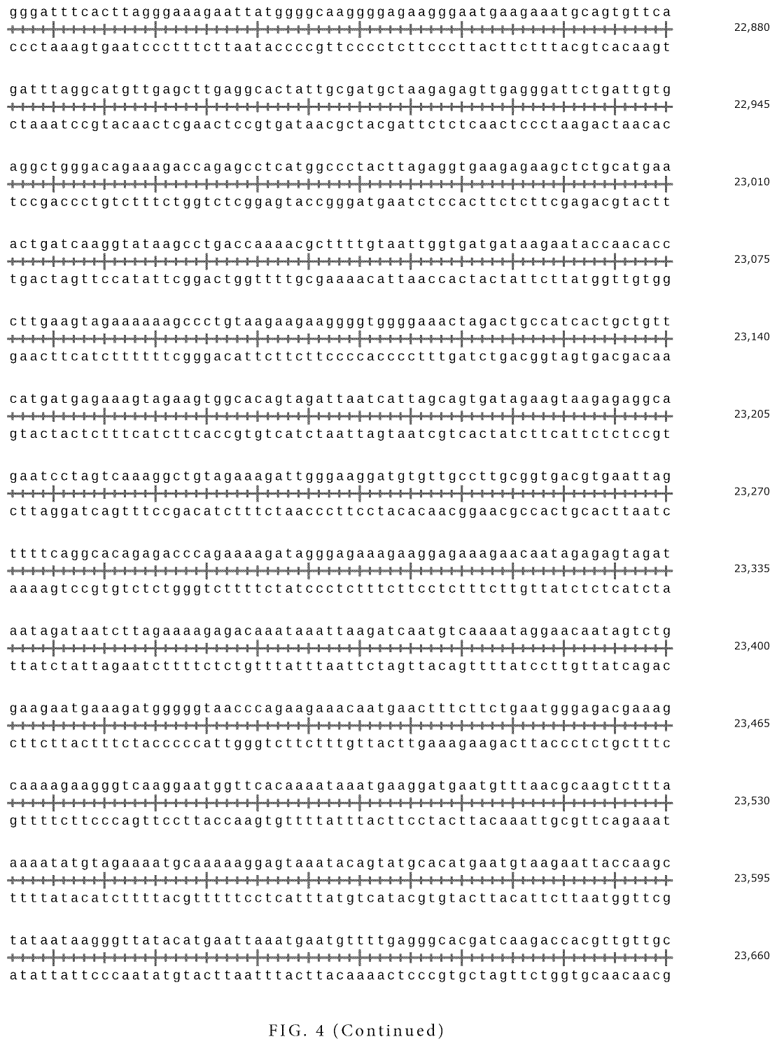

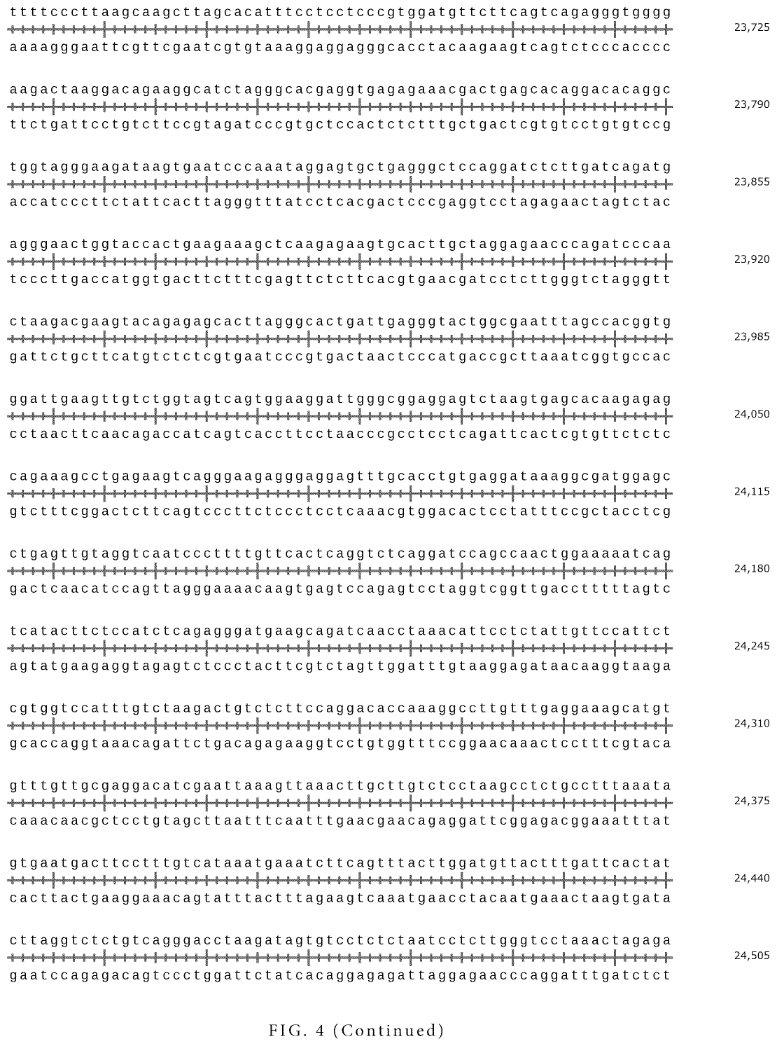

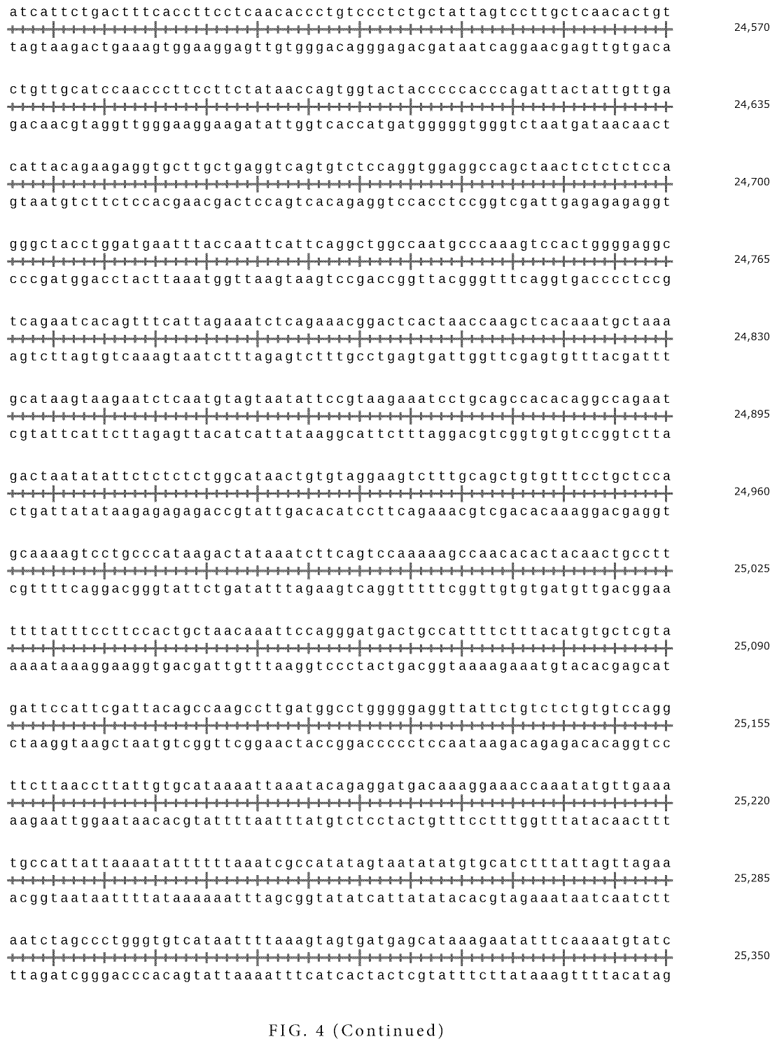

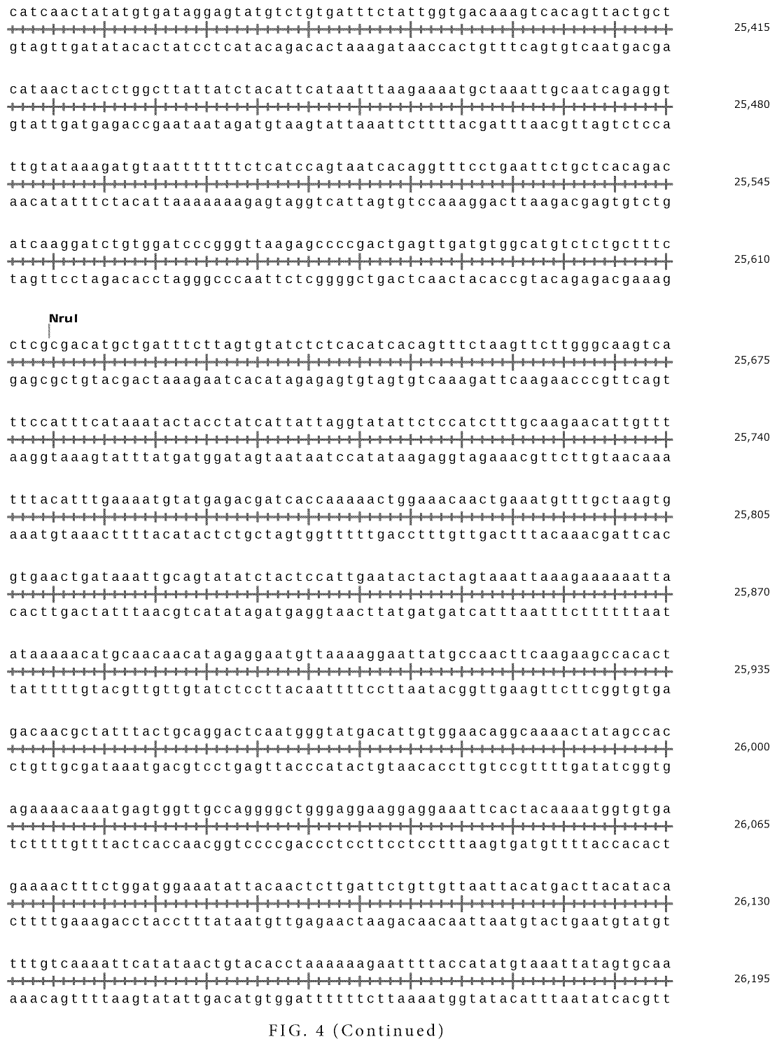

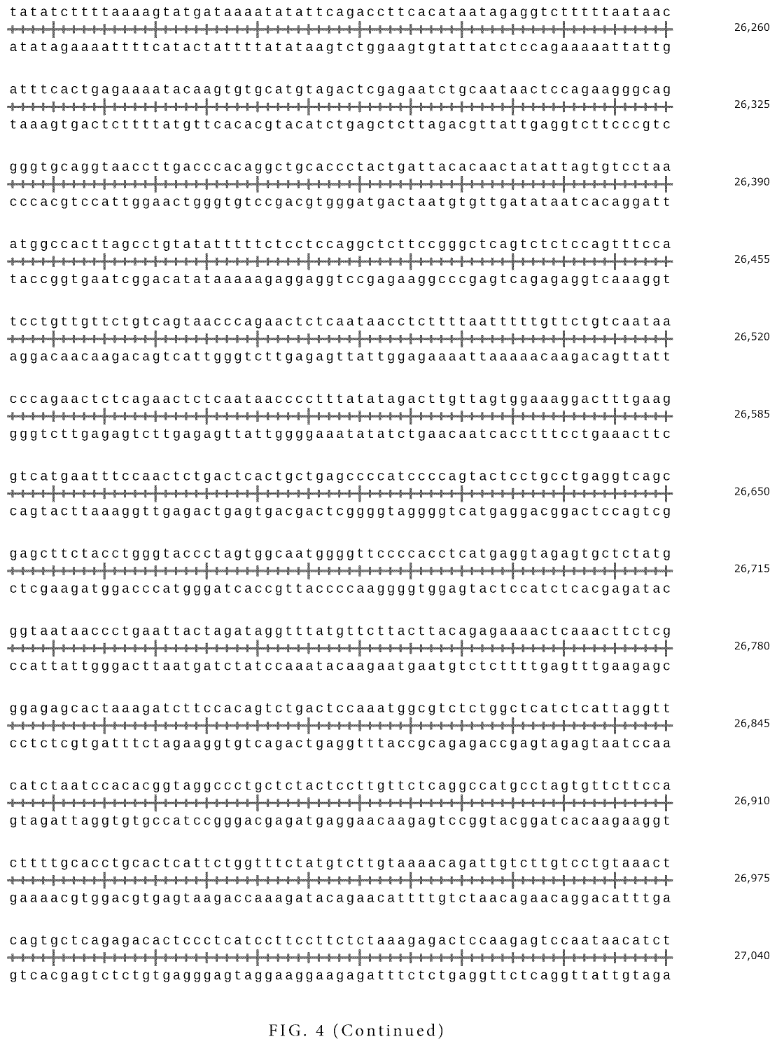

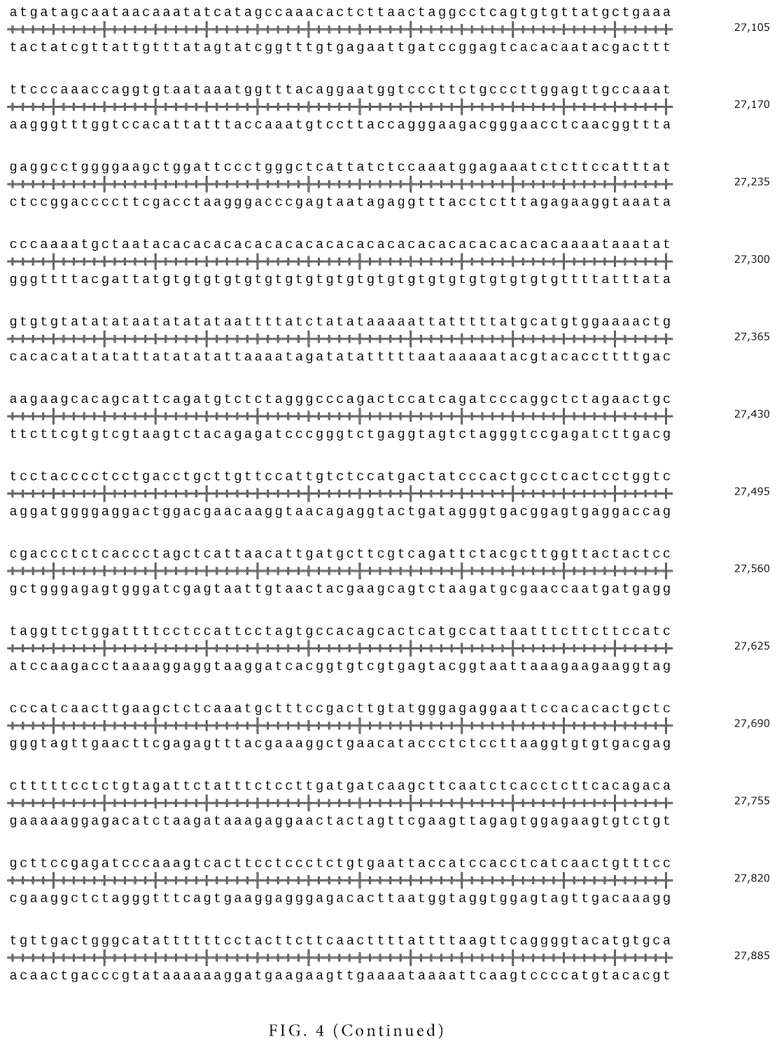

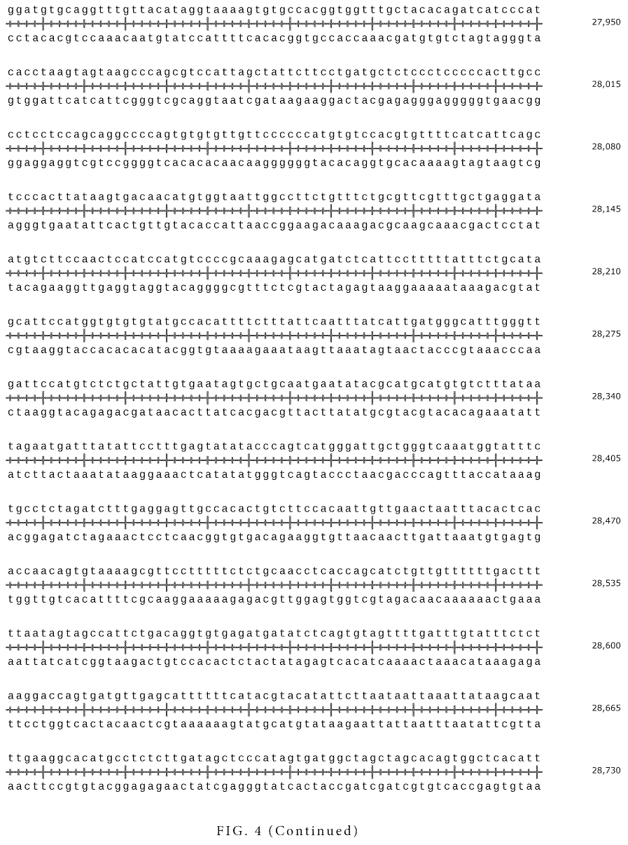

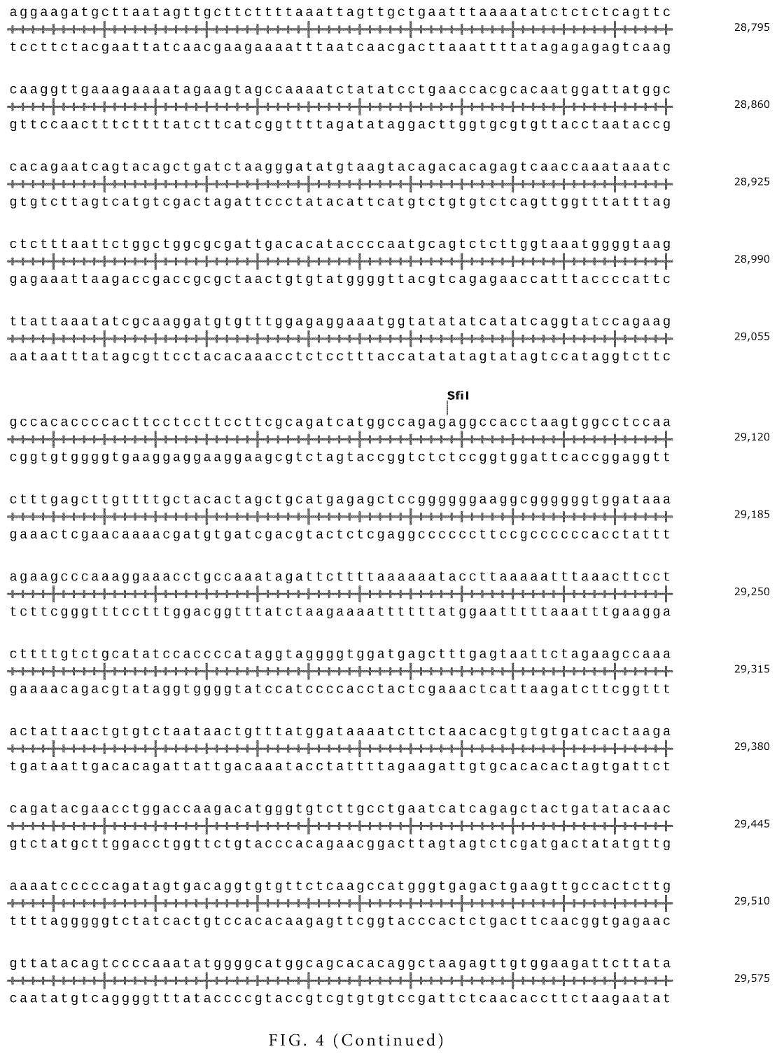

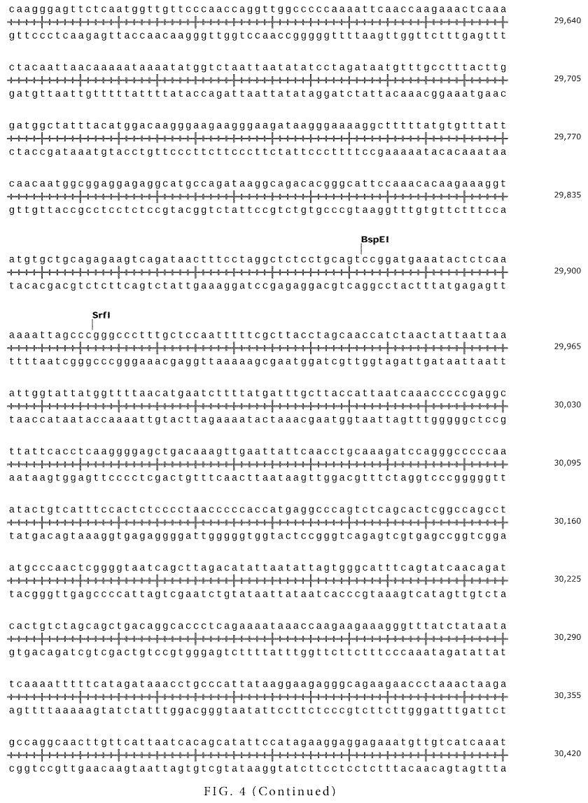

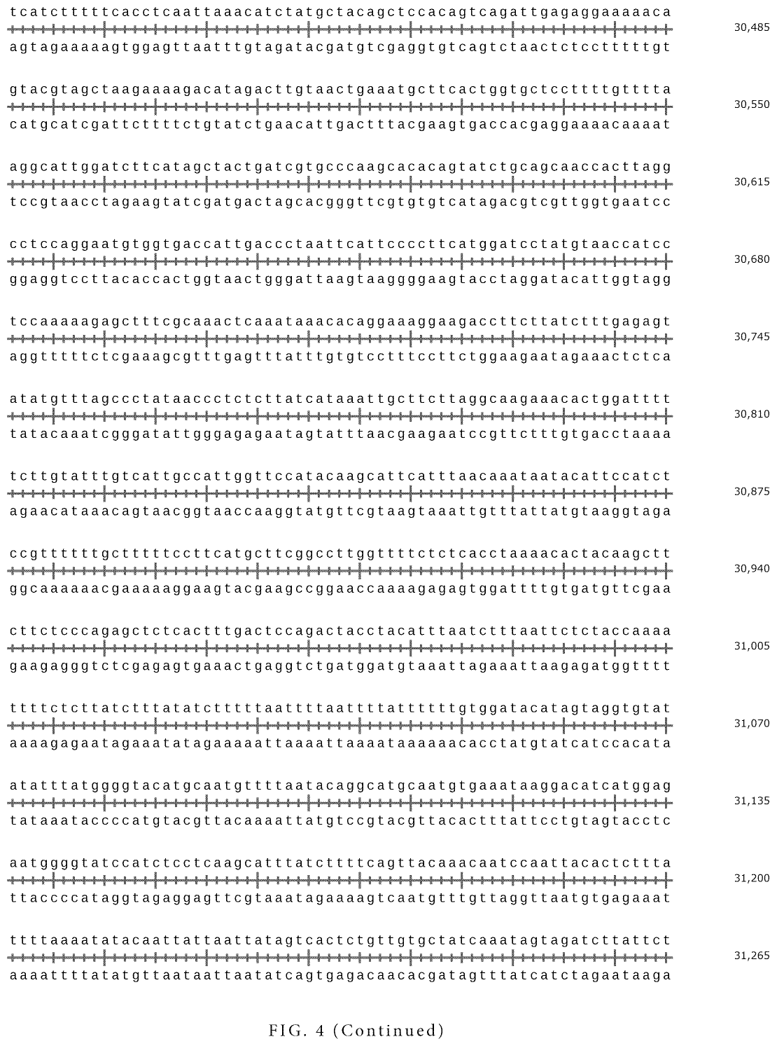

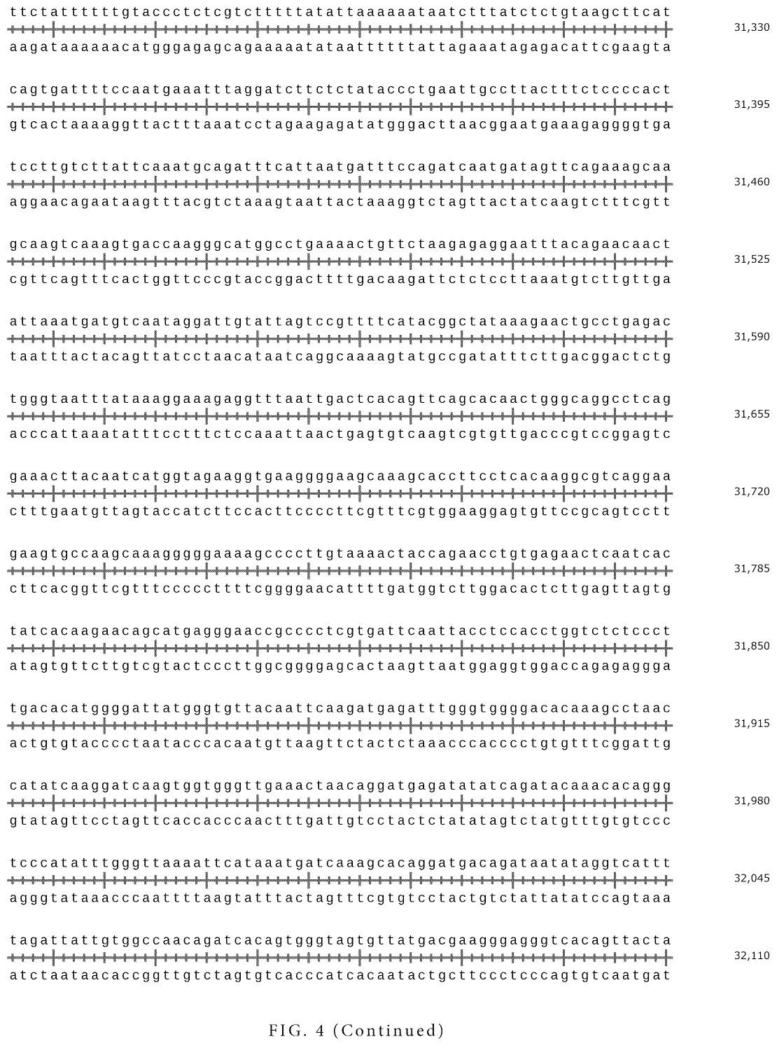

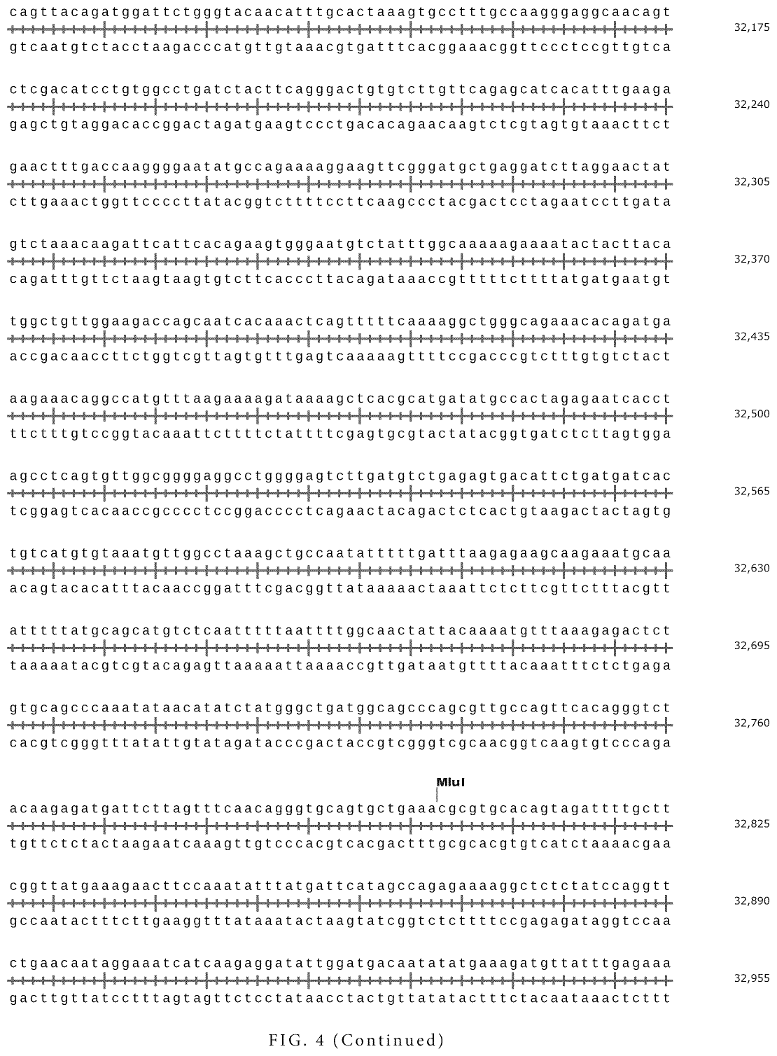

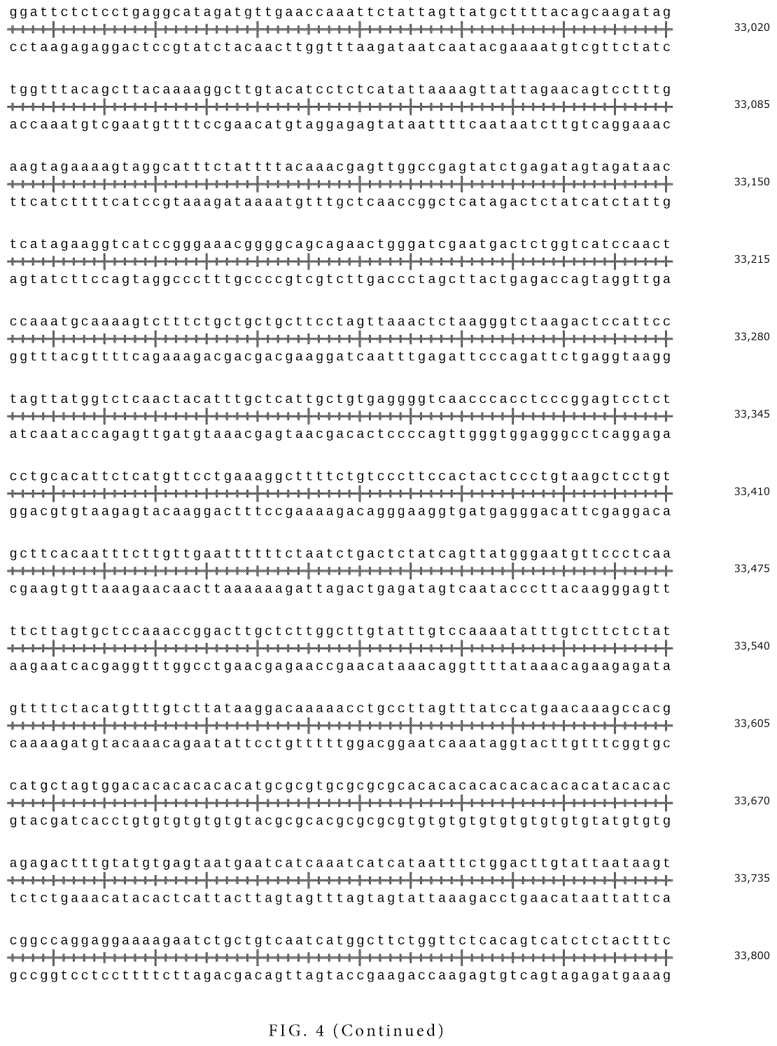

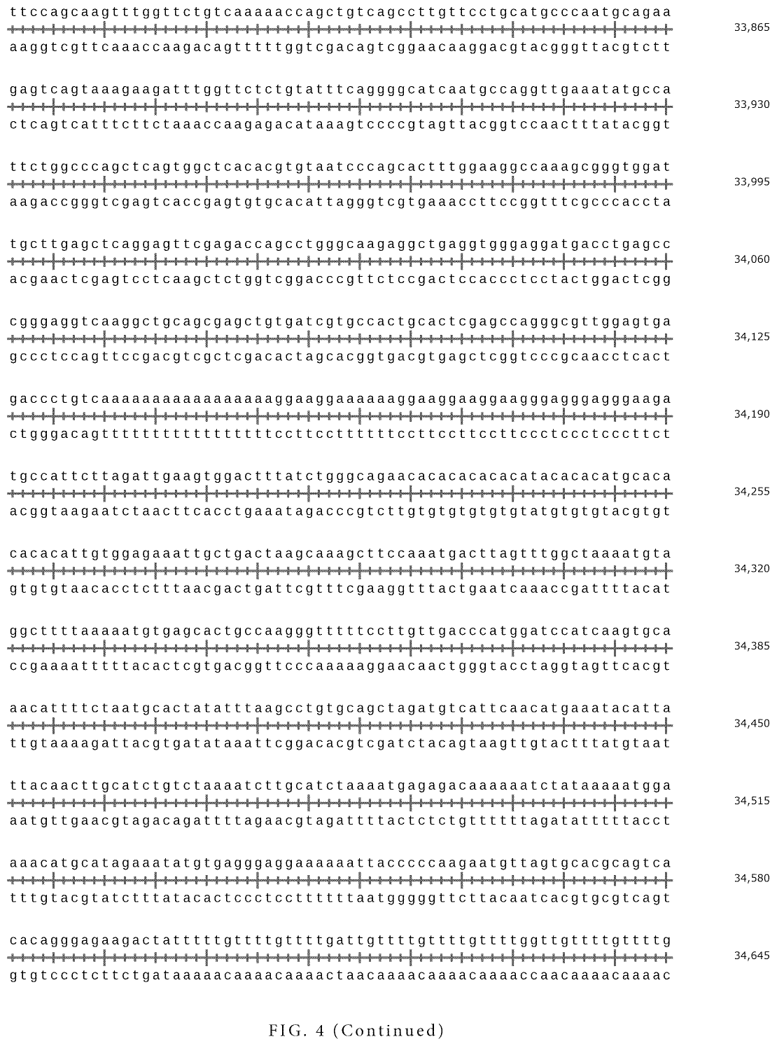

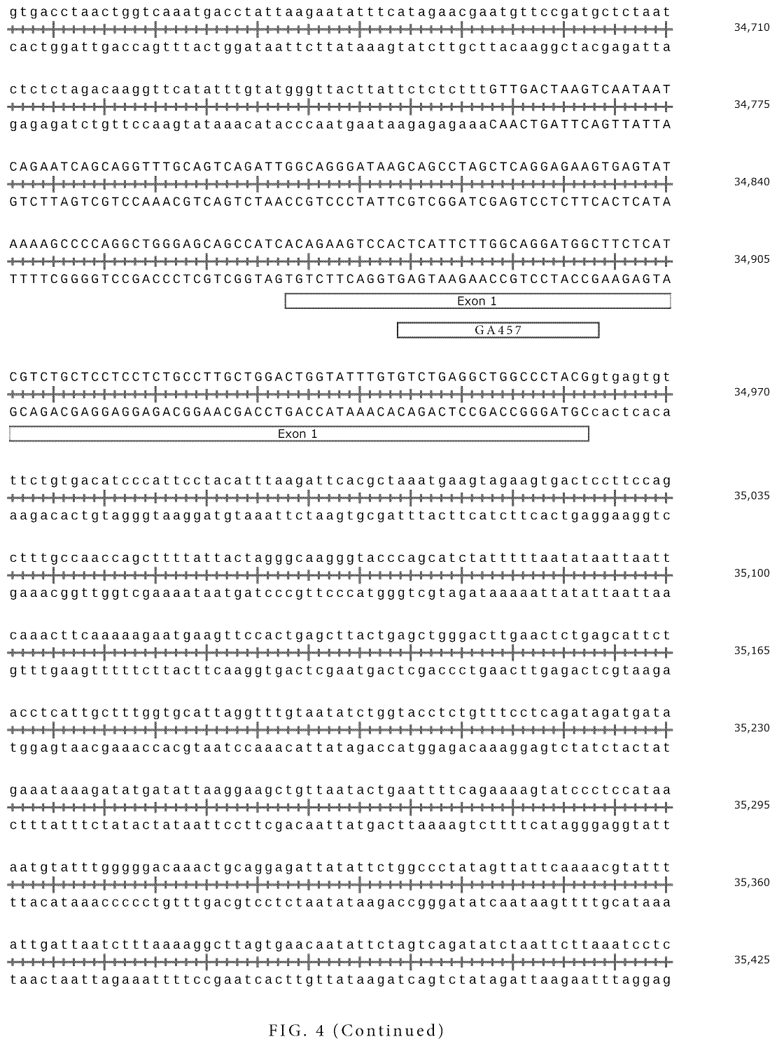

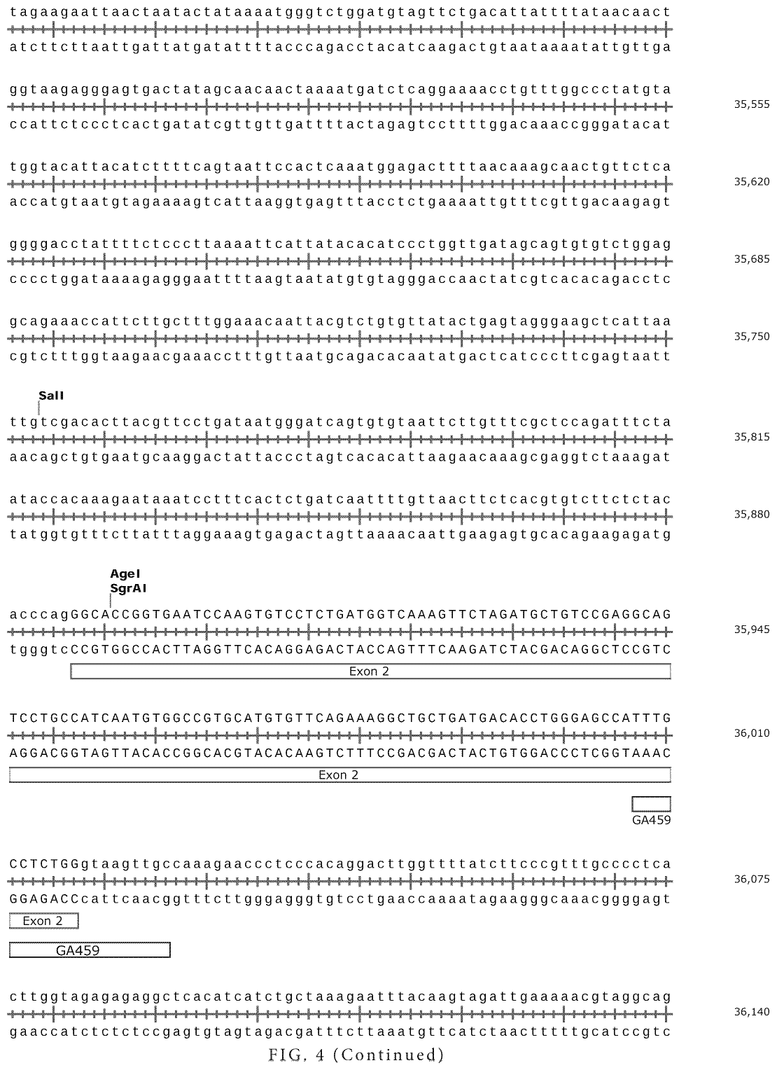

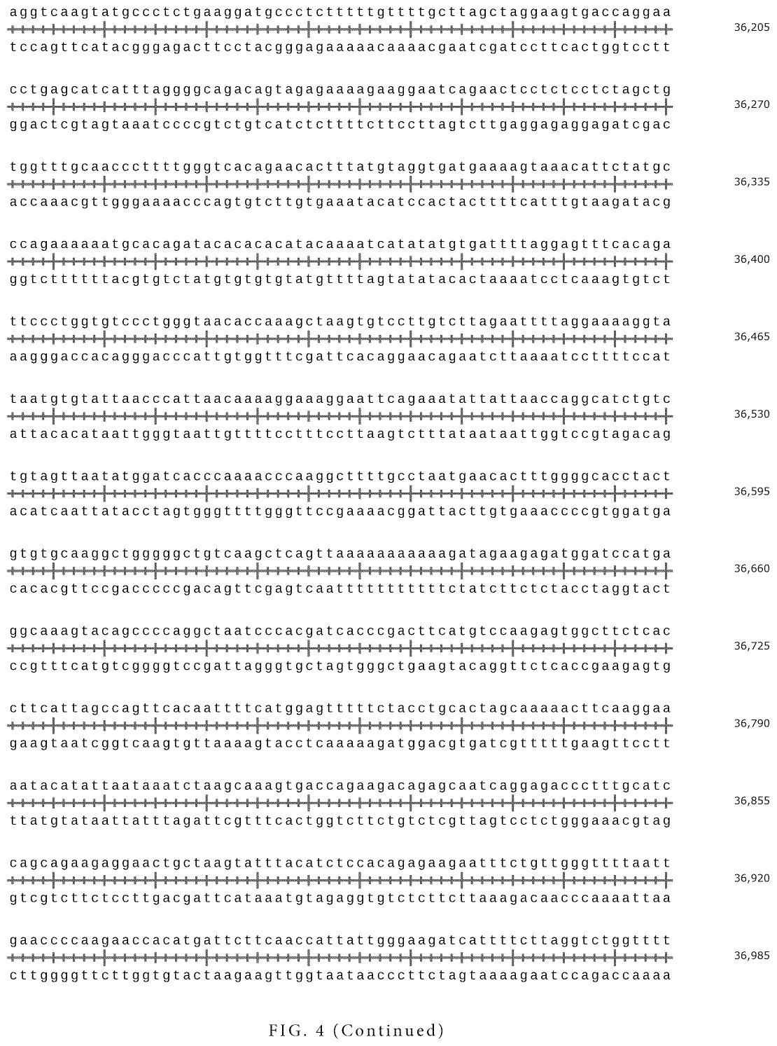

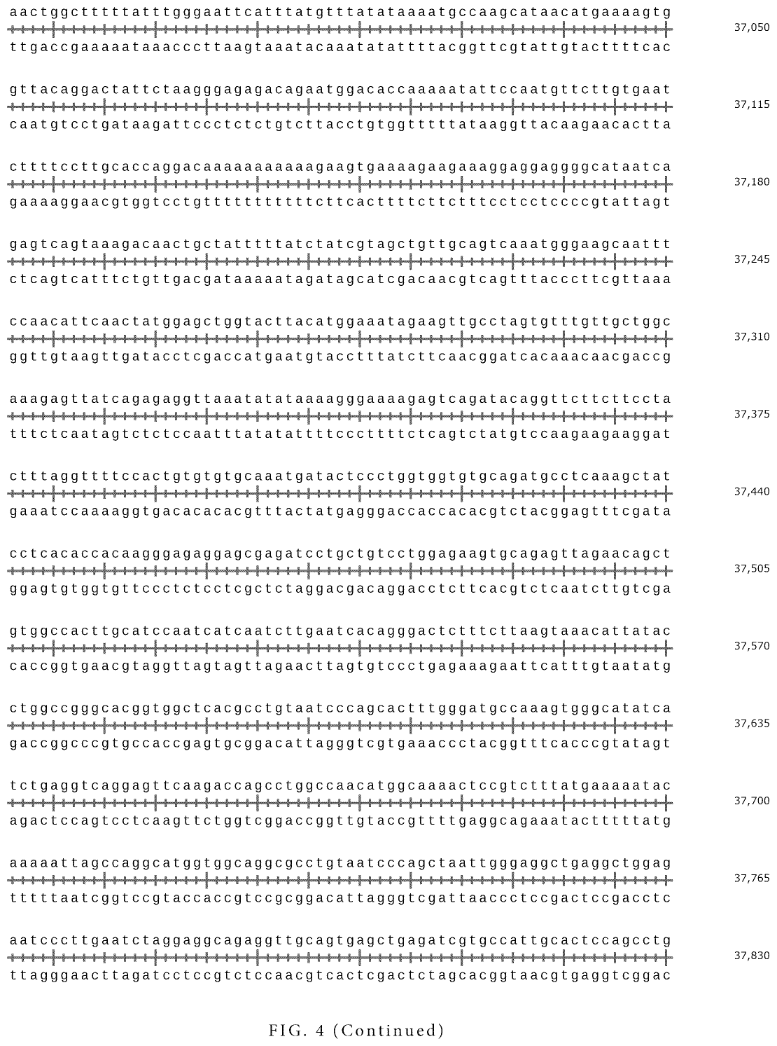

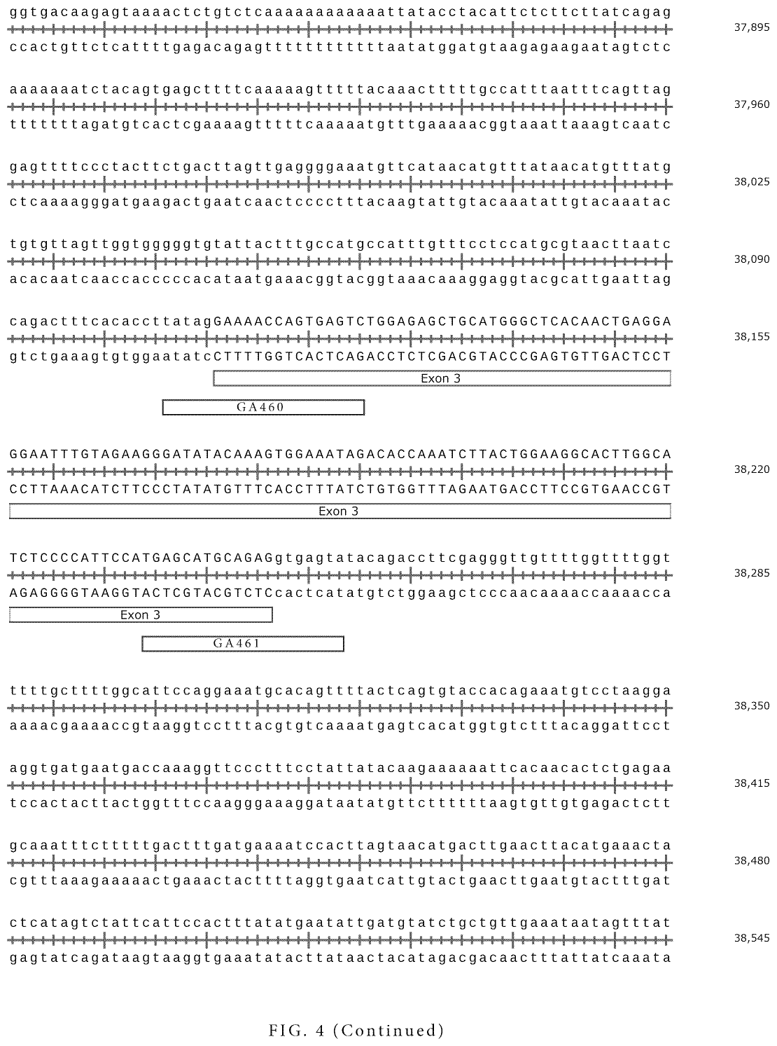

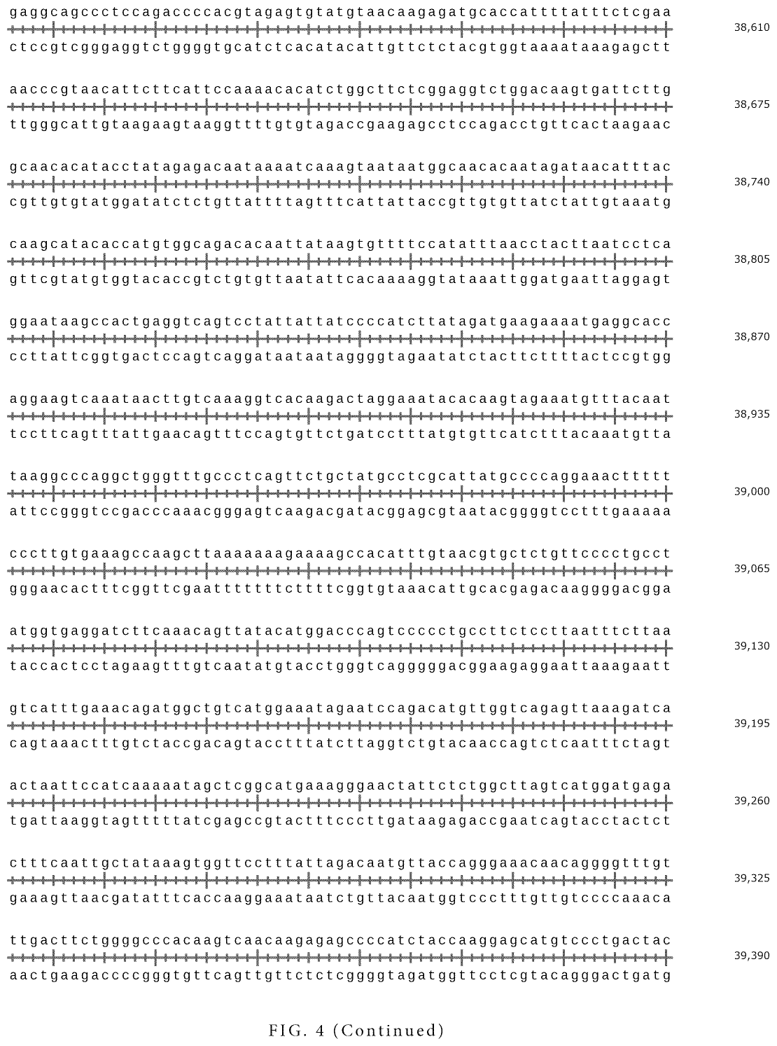

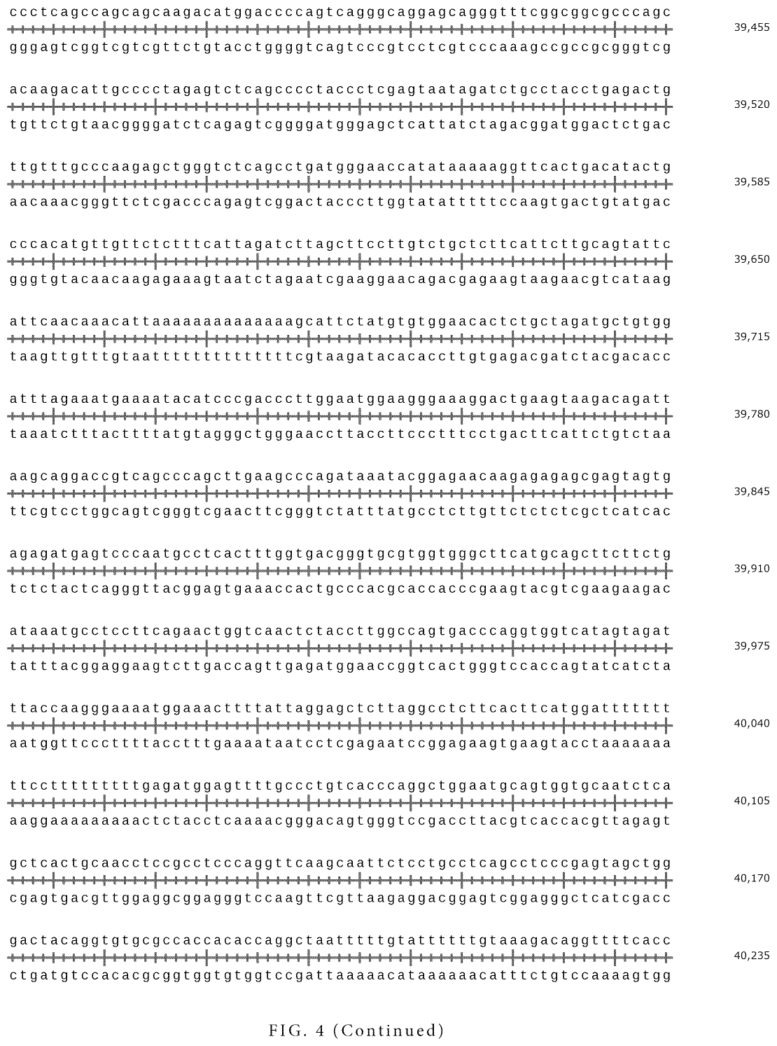

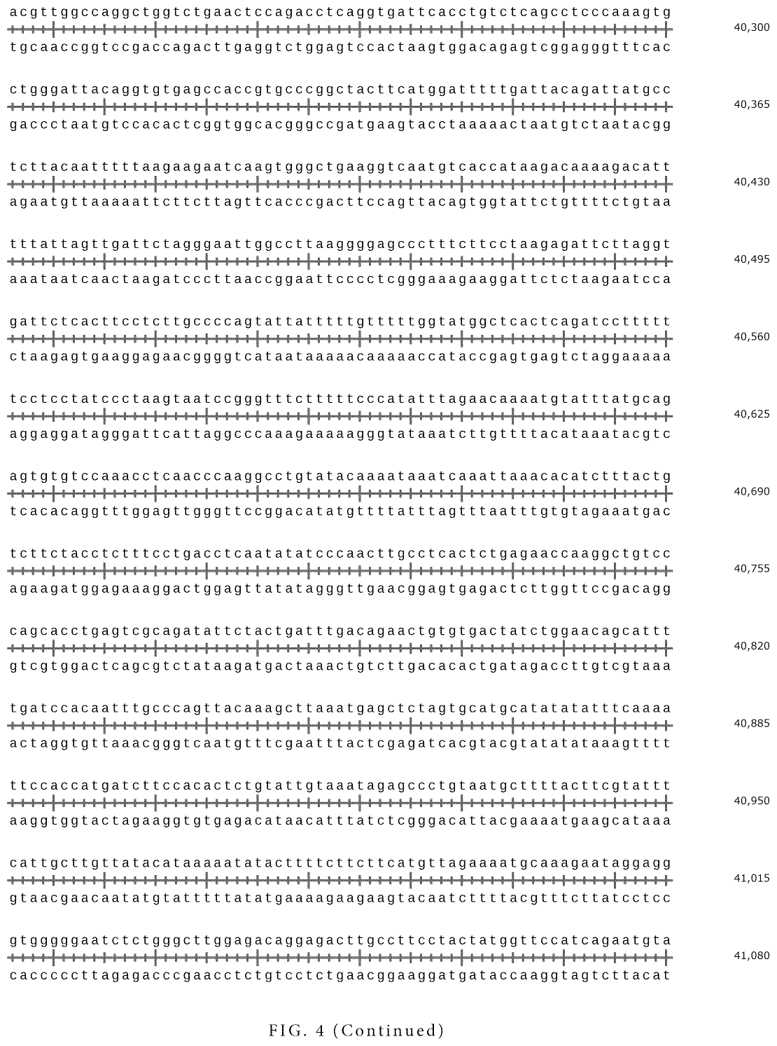

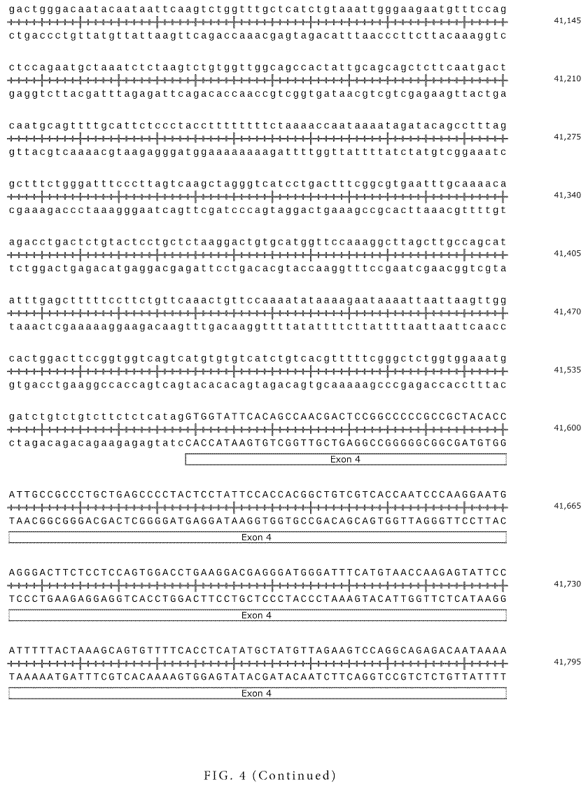

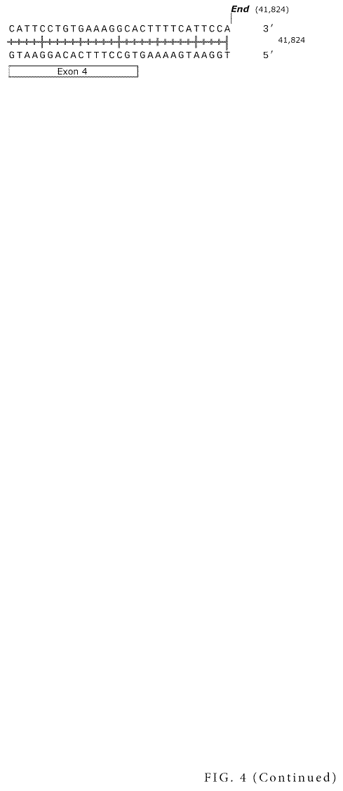

The TTR gene, composed of four exons, is located on chromosome 18 at 18q12.1. The full sequence of the human TTR gene is shown in FIG. 4 and is also available at UniProtKB-P02766 (TTHY_HUMAN). Over 120 TTR variants have so far been identified, the great majority of which are pathogenic. The most common pathogenic variant consists of a point mutation leading to replacement of valine by methionine at position 30 of the mature protein. This Val30Met mutation is responsible for hATTR amyloidosis and is the most frequent amyloidogenic mutation worldwide, accounting for about 50% of TTR variants.

Hereditary transthyretin amyloidosis (hATTR) is a disease caused by mutations in the TTR gene. Autosomal dominant mutations destabilize the TTR tetramer and enhance dissociation into monomers, resulting in misfolding, aggregation, and the subsequent extracellular deposition of TTR amyloid fibrils in different tissue sites. This multisystem extracellular deposition of amyloid (amyloidosis) results in dysfunction of different organs and tissues. In particular, polyneuropathy due to transthyretin amyloidosis (ATTR-PN) and cardiomyopathy due to transthyretin amyloidosis (ATTR-CM) are severe disorders associated with significant morbidity and mortality.

When there is clinical suspicion for hATTR-PN, diagnosis is typically done by tissue biopsy with staining for amyloid, amyloid typing (using immunohistochemistry or mass spectrometry), and/or TTR gene sequencing. When there is clinical suspicion for ATTR-CM, the key diagnostic tools are either endomyocardial biopsy (with tissue staining and amyloid typing by immunohistochemistry or mass spectrometry) or 99mtechnetium-pyrophosphate scan. Both of these approaches can provide a diagnosis of ATTR-CM. TTR gene sequencing can be used to differentiate between the hATTR-CM (mutation positive) and ATTRwt-CM (mutation negative).

The compositions described herein include a spacer having a nucleotide sequence that functions as a guide to direct a gene editing protein (e.g., a base editor) to alter the TTR gene, for example by introducing one or more nucleobase alterations in the TTR gene. These point mutations may be used to disrupt gene function, by the introduction of a missense mutation(s) that results in production of a less functional, or non-functional protein, thus silencing the TTR gene. Alternatively, it is contemplated herein that corrections to one or more point mutation(s) may be made using a gene editing protein to alter a mutated gene to correct the underlying mutation causing the dysfunction in the TTR gene or otherwise mitigate against dysfunction of the gene.

### Amyloidosis

Amyloidosis is a disorder that involved extracellular deposition of amyloid in an organ or tissue (e.g., the liver). Amyloidosis can occur when mutant transthyretin polypeptides aggregate (e.g., as fibrils). An amyloidosis caused by a mutation to the transthyretin gene can be referred to as a “transthyretin amyloidosis”. Some forms of transthyretin amyloidosis are not associated with a mutation to the transthyretin gene. Non-limiting examples of mutations to the mature transthyretin (TTR) protein that can lead to amyloidosis include the alterations T60A, V30M, V30A, V30G, V30L, V122I, V122A, and V122 (-). One method for treatment of transthyretin amyloidosis includes disrupting expression or activity of transthyretin in a cell of a subject, optionally a hepatocyte cell. Accordingly, provided herein are methods for reducing or eliminating expression of transthyretin in a cell. The transthyretin in the cell can be a pathogenic variant. Expression of transthyretin in a cell can be disrupted by disrupting splicing of a transthyretin transcript.

Transthyretin amyloidosis is a progressive condition characterized by the buildup of protein deposits in organs and/or tissues. These protein deposits can occur in the peripheral nervous system, which is made up of nerves connecting the brain and spinal cord to muscles and sensory cells that detect sensations such as touch, pain, heat, and sound. Protein deposits in these nerves result in a loss of sensation in the extremities (peripheral neuropathy). The autonomic nervous system, which controls involuntary body functions such as blood pressure, heart rate, and digestion, may also be affected by amyloidosis. In some cases, the brain and spinal cord (i.e., central nervous system) are affected. Other areas of amyloidosis include the heart, kidneys, eyes, liver, and gastrointestinal tract. The age at which symptoms begin to develop can be between the ages of 20 and 70.

There are three major forms of transthyretin amyloidosis, which are distinguished by their symptoms and the body systems they effect: neuropathic, leptomeningeal, and cardiac.

The neuropathic form of transthyretin amyloidosis primarily affects the peripheral and autonomic nervous systems, resulting in peripheral neuropathy and difficulty controlling bodily functions. Impairments in bodily functions can include sexual impotence, diarrhea, constipation, problems with urination, and a sharp drop in blood pressure upon standing (orthostatic hypotension). Some people experience heart and kidney problems as well. Various eye problems may occur, such as cloudiness of the clear gel that fills the eyeball (vitreous opacity), dry eyes, increased pressure in the eyes (glaucoma), or pupils with an irregular or “scallope” d appearance. Some people with this form of transthyretin amyloidosis develop carpal tunnel syndrome, which can involve numbness, tingling, and weakness in the hands and fingers.

The leptomeningeal form of transthyretin amyloidosis primarily affects the central nervous system. In people with this form, amyloidosis occurs in the leptomeninges, which are two thin layers of tissue that cover the brain and spinal cord. A buildup of protein in this tissue can cause stroke and bleeding in the brain, an accumulation of fluid in the brain (hydrocephalus), difficulty coordinating movements (ataxia), muscle stiffness and weakness (spastic paralysis), seizures, and loss of intellectual function (dementia). Eye problems similar to those in the neuropathic form may also occur. When people with leptomeningeal transthyretin amyloidosis have associated eye problems, they are said to have the oculoleptomeningeal form.

The cardiac form of transthyretin amyloidosis affects the heart. People with cardiac amyloidosis may have an abnormal heartbeat (arrhythmia), an enlarged heart (cardiomegaly), or orthostatic hypertension. These abnormalities can lead to progressive heart failure and death. Occasionally, people with the cardiac form of transthyretin amyloidosis have mild peripheral neuropathy.

Mutations in the transthyretin (TTR) gene cause transthyretin amyloidosis. Transthyretin transports vitamin A (retinol) and a hormone called thyroxine throughout the body. Not being bound by theory, to transport retinol and thyroxine, transthyretin must form a tetramer. Transthyretin is produced primarily in the liver (i.e., in hepatic cells). A small amount of transthyretin (TTR) is produced in an area of the brain called the choroid plexus and in the retina.

TTR gene mutations can alter the structure of transthyretin, impairing its ability to bind to other transthyretin proteins. The TTR gene mutation can be autosomal dominant.

### Splice Sites

Gene splice sites and splice site motifs are well known in the art and it is within the skill of a practitioner to identify splice sites in sequence (see, e.g., Sheth, et al., “Comprehensive splice-site analysis using comparative genomics”, Nucleic Acids Research, 34:3955-3967 (2006); Dogan, et al., “AplicePort—an interactive splice-site analysis tool”, Nucleic Acids Research, 35: W285-W291 (2007); and Zuallaert, et al., “SpliceRover: interpretable convolutional neural networks for improved splice site prediction”, Bioinformatics, 34:4180-4188 (2018)).

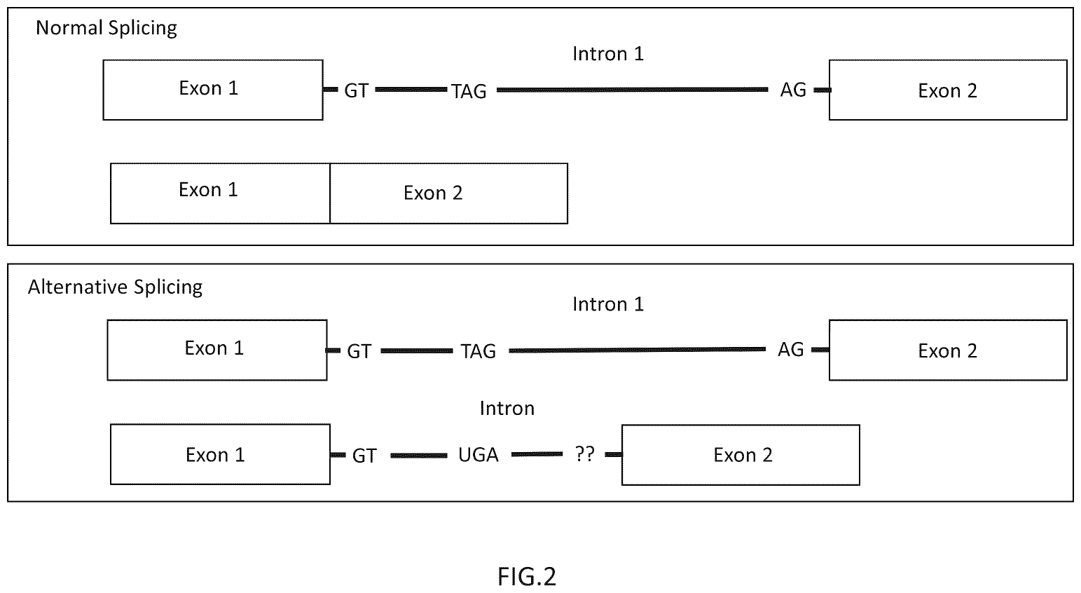

As shown in FIG. 2, canonical splice donors comprise the DNA sequence GT on the sense strand, whereas canonical splice acceptors comprise the DNA sequence AG. Alteration of the sequence disrupts normal splicing. Splice donors can be disrupted by adenine base editing of the complementary base in the second position in the antisense strand (GT→GC), and splice acceptors can be disrupted by adenine base editing of the first position in the sense strand (AG→GG).

### Editing of Target Genes

To edit the transthyretin (TTR) gene, a cell (e.g., a hepatocyte) is contacted with a guide RNA and a nucleobase editor polypeptide comprising a nucleic acid programmable DNA binding protein (napDNAbp) and a cytidine deaminase or adenosine deaminase to edit a base of a gene sequence. Editing of the base can result in disruption of a splice site (e.g, through alteration of a splice-site motif nucleobase). Editing of the base can result in replacement of a pathogenic variant amino acid with a non-pathogenic variant amino acid. As a non-limiting example, editing of the base can result in replacing a T60A, V30M, V30A, V30G, V30L, V122I, V122A, or a V122 (-) alteration in the mature transthyretin (TTR) polypeptide with a non-pathogenic variant or the wild-type valine residue. The cytidine deaminase can be BE4 (e.g., saBE4). The adenosine deaminase can be ABE (e.g., saABE.8.8). In some embodiments, multiple target sites are edited simultaneously. In some embodiments, the TTR gene is edited by contacting a cell with a nuclease and a guide RNA to introduce an indel into a gene sequence. The indel can be associated with a reduction or elimination of expression of the gene. The nuclease can be Cas12b (e.g., bhCas12b). The cells can be edited in vivo or ex vivo. The guide RNA can be a single guide or a dual guide. In some embodiments, cells to be edited are contacted with at least one nucleic acid, wherein at least one nucleic acid encodes a guide RNA, or two or more guide RNAs, and a nucleobase editor polypeptide comprising a nucleic acid programmable DNA binding protein (napDNAbp) and a deaminase, e.g., an adenosine or a cytidine deaminase. In some embodiments, the gRNA comprises nucleotide analogs. These nucleotide analogs can inhibit degradation of the gRNA by cellular processes. Exemplary single guide RNA (sgRNA) sequences are provided in Tables 1, 8, 20, 27, and 28 and exemplary spacer sequences and target sequences (e.g., protospacer sequences) are provided in Tables 2A, 2B, 2C, 9, 10, 20, 25, 29, and 30.

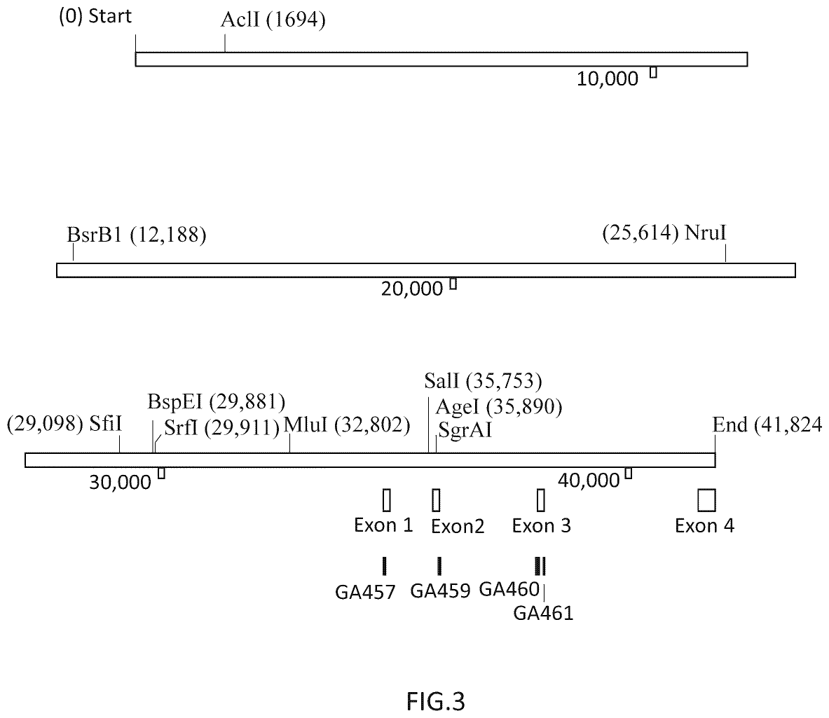

With the present disclosure, protospacer sequences were identified within the nucleotide sequence of the human TTR gene to be used as guide sequences that permit ABE8.8 (and other ABE variants containing Streptococcus pyogenes Cas9, such as ABE7.10, or another Cas protein that can use the NGG PAM) to either disrupt the start codon, or disrupt splice sites, whether donors or acceptors, via A→G editing within its editing window (roughly positions 4 to 7 in the 20-nt protospacer region of DNA). Four of the sequences shown in Table 8 were identified within the human TTR gene. The alignment of these four protospacer sequences on a map of the human TTR gene is shown in FIG. 3.

Protospacer, corresponding to guide RNA GA457, has the sequence 5′-GCCATCCTGCCAAGAATGAG-3′ (SEQ ID NO: 467) and is located at 34,879 to 34,898 bp of the human TTR gene.

Protospacer, corresponding to guide RNA GA459, has the sequence 5′-GCAACTTACCCAGAGGCAAA-3′ (SEQ ID NO: 468) and is located at 36,007 to 36,026 bp of the human TTR gene.

Protospacer, corresponding to guide RNA GA460, has the sequence 5′-TATAGGAAAACCAGTGAGTC-3′ (SEQ ID NO: 469) and is located at 38,106-38,125 bp of the human TTR gene.

Protospacer, corresponding to guide RNA GA461, has the sequence 5′-TACTCACCTCTGCATGCTCA-3′ (SEQ ID NO: 470) and is located at 38,234-38253 of the human TTR gene.

Protospacer, corresponding to guide RNA GA458, has the sequence 5′-GCCATCCTGCCAAGAACGAG-3′ (SEQ ID NO: 471) represents the sequence within the cynomolgus macaque TTR gene corresponding to the human protospacer sequence corresponding to guide RNA GA459.

The present disclosure includes a guide polynucleotide having a sequence at least about 75%, at least about 80%, at least about 85%, at least about 90%, or at least about 95% identical to the sequence 5′-GCCAUCCUGCCAAGAAUGAG-3′ (SEQ ID NO: 472) (GA457). The present disclosure includes a guide polynucleotide having the sequence 5′-GCCAUCCUGCCAAGAAUGAG-3′ (SEQ ID NO: 472) (GA457).

The present disclosure includes a modified guide polynucleotide having a sequence at least about 75%, at least about 80%, at least about 85%, at least about 90%, or at least about 95% identical to the sequence 5′-GCCAUCCUGCCAAGAAUGAG-3′ (SEQ ID NO: 472), wherein GCC are modified by methylation (GA521) (C is modified to 2′-O-methylcytidine, G is modified to 2′-O-methylguanosine). The present disclosure includes a modified guide polynucleotide having the sequence 5′-mGsmCsmCAUCCUGCCAAGAAUGAG-3′ (SEQ ID NO: 472) (GA521), wherein mC: 2′-O-methylcytidine, mG: 2′-O-methylguanosine and s: phosphorothioate (PS) backbone linkage.

The present disclosure includes a guide polynucleotide having a sequence at least about 75%, at least about 80%, at least about 85%, at least about 90%, or at least about 95% identical to the sequence 5′-GCCAUCCUGCCAAGAACGAG-3′ (SEQ ID NO: 473) (GA458). The present disclosure includes a guide polynucleotide having the sequence 5′-GCCAUCCUGCCAAGAACGAG-3′ (SEQ ID NO: 473) (GA458).

The present disclosure includes a guide polynucleotide having a sequence at least about 75%, at least about 80%, at least about 85%, at least about 90%, or at least about 95% identical to the sequence 5′-GCAACUUACCCAGAGGCAAA-3′ (SEQ ID NO: 474) (GA459). The present disclosure includes a guide polynucleotide having the sequence 5′-GCAACUUACCCAGAGGCAAA-3′ (SEQ ID NO: 474) (GA459).

The present disclosure includes a guide polynucleotide having a sequence at least about 75%, at least about 80%, at least about 85%, at least about 90%, or at least about 95% identical to the sequence 5′-UAUAGGAAAACCAGUGAGUC-3′ (SEQ ID NO: 475) (GA460). The present disclosure includes a guide polynucleotide having the sequence 5′-UAUAGGAAAACCAGUGAGUC-3′ (SEQ ID NO: 475) (GA460).

The present disclosure includes a guide polynucleotide having a sequence at least about 75%, at least about 80%, at least about 85%, at least about 90%, or at least about 95% identical to the sequence 5′-UACUCACCUCUGCAUGCUCA-3′ (SEQ ID NO: 476) (GA461). The present disclosure includes a guide polynucleotide having the sequence 5′-UACUCACCUCUGCAUGCUCA-3′ (SEQ ID NO: 476) (GA461).

In some aspects, provided herein is a guide RNA, comprising a sequence defined by mG*mC*mC*AUCCUGCCAAGAAUGAGmGUUUUAGmAmGmCmUmAGmAmAmAmUmAmGmCmAm AGUUmAAmAAmUAmAmGmGmCmUmAGUmCmCGUUAmUmCAAmCmUmUGmAmAmAmAmAmGmU mGGmCmAmCmCmGmAmGmUmCmGmGmUmGmCmU*mU*mU*mU (GA521, (SEQ ID NO: 477), wherein A is adenosine, C is cytidine, G is guanosine, U is uridine, mA*is 2′-O-methyladenosine, mC*is 2′-O-methylcytidine, mG*is 2′-O-methylguanosine, mU*is 2′-O-methyluridine, and wherein nucleotides represented in bold are linked by a phosphorothioate (PS) backbone linkage.

Alternatively, GA521 is represented as

mG*smC*smC*AUCCUGCCAAGAAUGAGmGsUsUsUsUsAsGsmAsmGsmCsmUsmA SGsmAsmAsmAsmUsmAsmGsmCssmAsmAsGsUsUsmAsAsmAsAsmUsAsmAsmGsmGsm CsmUsmAsGsUsmCsmCsGsUsUsAsmUsmCsAsAsmCsmUsmUsGsmAsmAsmAsmAsmAs mGsmUsmGsGsmCsmAsmCsmCsmGsmAsmGsmUsmCsmGsmGsmUsmGsmCsmU*smU*sm U*smU (GA521, SEQ ID NO: 477), wherein A is adenosine, C is cytidine, G is guanosine, U is uridine, mA*is 2′-O-methyladenosine, mC*is 2′-O-methylcytidine, mG*is 2′-O-methylguanosine, mU*is 2′-O-methyluridine, and wherein nucleotides are linked by a phosphorothioate (PS) backbone linkage represented by the letter ‘s’.

In some embodiments,

mG*mC*mC*AUCCUGCCAAGAAUGAGmGUUUUAGmAmGmCmUmAGmAmAmAmUmAmG mCmAmAGUUmAAmAAmUAmAmGmGmCmUmAGUmCmCGUUAmUmCAAmCmUmUGmAmAmAmAm AmGmUmGGmCmAmCmCmGmAmGmUmCmGmGmUmGmCmU*mU*mU*mU (GA521, SEQ ID NO: 477), wherein A is adenosine, C is cytidine, G is guanosine, U is uridine, mA*is 2′-O-methyladenosine, mC*is 2′-O-methylcytidine, mG*is 2′-O-methylguanosine, mU*is 2′-O-methyluridine, and wherein nucleotides represented in bold are linked by a phosphorothioate (PS) backbone linkage.

Alternatively, GA521 is represented as

mG*mC*mC*AUCCUGCCAAGAAUGAGmGsUsUsUsUsAsGsmAsmsGsmCsmUsmAs GsmAsmAsmAsmUsmAsmGsmCssmAsmAsGsUsUsmAsAsmAsAsmUsAsmAsmGsmGsmC smUsmAsGsUsmCsmCsGsUsUsAsmUsmCsAsAsmCsmUsmUsGsmAsmAsmAsmAsmAsm GsmUsmGsGsmCsmAsmCsmCsmGsmAsmGsmUsmCsmGsmGsmUsmGsmCsmU*mU*mU*m U (GA521, SEQ ID NO: 478), wherein A is adenosine, C is cytidine, G is guanosine, U is uridine, mA*is 2′-O-methyladenosine, mC*is 2′-O-methylcytidine, mG*is 2′-O-methylguanosine, mU*is 2′-O-methyluridine, and wherein nucleotides represented in bold are linked by a phosphorothioate (PS) backbone linkage represented by the letter ‘s’.

In various instances, it is advantageous for a spacer sequence to include a 5′ and/or a 3′ “G” nucleotide. In some cases, for example, any spacer sequence or guide polynucleotide provided herein comprises or further comprises a 5′ “G”, where, in some embodiments, the 5′ “G” is or is not complementary to a target sequence. In some embodiments, the 5′ “G” is added to a spacer sequence that does not already contain a 5′ “G.” For example, it can be advantageous for a guide RNA to include a 5′ terminal “G” when the guide RNA is expressed under the control of a U6 promoter or the like because the U6 promoter prefers a “G” at the transcription start site (see Cong, L. et al. “Multiplex genome engineering using CRISPR/Cas systems. Science 339:819-823 (2013) doi: 10.1126/science.1231143). In some cases, a 5′ terminal “G” is added to a guide polynucleotide that is to be expressed under the control of a promoter, but is optionally not added to the guide polynucleotide if or when the guide polynucleotide is not expressed under the control of a promoter.

Exemplary guide RNAs, spacer sequences, and target sequences are provided in Tables 1, 2A, 2B, 2C, 9, 10, 20, 25, and 27-30.

In various instances, it is advantageous for a spacer sequence to include a 5′ and/or a 3′ “G” nucleotide. In some cases, for example, any spacer sequence or guide polynucleotide provided herein comprises or further comprises a 5′ “G”, where, in some embodiments, the 5′ “G” is or is not complementary to a target sequence. In some embodiments, the 5′ “G” is added to a spacer sequence that does not already contain a 5′ “G.” For example, it can be advantageous for a guide RNA to include a 5′ terminal “G” when the guide RNA is expressed under the control of a U6 promoter or the like because the U6 promoter prefers a “G” at the transcription start site (see Cong, L. et al. “Multiplex genome engineering using CRISPR/Cas systems. Science 339:819-823 (2013) doi: 10.1126/science. 1231143). In some cases, a 5′ terminal “G” is added to a guide polynucleotide that is to be expressed under the control of a promoter, but is optionally not added to the guide polynucleotide if or when the guide polynucleotide is not expressed under the control of a promoter.

In embodiments, a guide RNA comprises a sequence complementary to a promoter region of a TTR polynucleotide sequence. In embodiments, the promoter region spans from positions +10, +5, +1, −1, −2, −3, −4, −5, −6, −7, −8, −9, −10, −15, −20, −25, −30, −35, −40, −45, −50, −55, −60, −65, −70, −75, −80, −85, −90, −95, −100, −105, −110, −115, −120, −125, −130, −135, −140, −145, −150, −155, −160, −165, −170, −175, −180, −185, −190, −195, −200, −250, or −300 to position +5, +1, −1, −2, −3, −4, −5, −6, −7, −8, −9, −10, −15, −20, −25, −30, −35, −40, −45, −50, −55, −60, −65, −70, −75, −80, −85, −90, −95, −100, −105, −110, −115, −120, −125, −130, −135, −140, −145, −150, −155, −160, −165, −170, −175, −180, −185, −190, −195, −200, −250, −300, or −400, where position +1 corresponds to the first A of the start codon (ATG) of the TTR polynucleotide sequence.

Variants of the spacer sequences listed in the following tables comprising 1, 2, 3, 4, or 5 nucleobase alterations are contemplated. For example, variation of a target polynucleotide sequence within a population (e.g., single nucleotide polymorphisms) may require said alterations to a spacer sequence to allow the spacer to better bind a variant of a target sequence in a subject.

TABLE 1
Exemplary guide RNAs for editing transthyretin
(TTR) splice sites and/or introducing indels into
the TTR gene (e.g., using bhCas12b)

| | | SEQ ID |
|---|---|---|
| sgRNA ID | Sequence | NO |
| sgRNA_361 | mUsmAsmUsAGGAAAACCAGTGAGTCGUUUUAGAGCUAGAAAUA | 479 |
| | GCAAGUUAAAAUAAGGCUAGUCCGUUAUCAACUUGAAAAAGUGG | |
| | CACCGAGUCGGUGCmUsmUsmUsU | |
| sgRNA_362 | mUsmAsmCsUCACCUCUGCAUGCUCAGUUUUAGAGCUAGAAAUA | 480 |
| | GCAAGUUAAAAUAAGGCUAGUCCGUUAUCAACUUGAAAAAGUGG | |
| | CACCGAGUCGGUGCmUsmUsmUsU | |
| sgRNA_363 | mAsmCsmUsCACCUCUGCAUGCUCAUGUUUUAGAGCUAGAAAUA | 481 |
| | GCAAGUUAAAAUAAGGCUAGUCCGUUAUCAACUUGAAAAAGUGG | |
| | CACCGAGUCGGUGCmUsmUsmUsU | |
| sgRNA_364 | mUsmAsmCsCACCUAUGAGAGAAGACGUUUUAGAGCUAGAAAUA | 482 |
| | GCAAGUUAAAAUAAGGCUAGUCCGUUAUCAACUUGAAAAAGUGG | |
| | CACCGAGUCGGUGCmUsmUsmUsU | |
| sgRNA_365 | mAsmUsmAsCUCACCUCUGCAUGCUCAGUUUUAGUACUCUGUAA | 483 |
| | UGAAAAUUACAGAAUCUACUAAAACAAGGCAAAAUGCCGUGUUU | |
| | AUCUCGUCAACUUGUUGGCGAGAmUsmUsmUsU | |
| sgRNA_366 | mAsmCsmUsGGUUUUCCUAUAAGGUGUGUUUUAGUACUCUGUAA | 484 |
| | UGAAAAUUACAGAAUCUACUAAAACAAGGCAAAAUGCCGUGUUU | |
| | AUCUCGUCAACUUGUUGGCGAGAmUsmUsmUsU | |
| sgRNA_367 | mGsmUsmUsCUGUCUUUUGGUCAGGACAACCGUCUAGCUAUAAG | 485 |
| | UGCUGCAGGGUGUGAGAAACUCCUAUUGCUGGACGAUGUCUCUU | |
| | ACGAGGCAUUAGCACUUGGCAGGAUGGCUUCUCmAsmUsmCsG | |
| sgRNA_368 | mGsmUsmUsCUGUCUUUUGGUCAGGACAACCGUCUAGCUAUAAG | 486 |
| | UGCUGCAGGGUGUGAGAAACUCCUAUUGCUGGACGAUGUCUCUU | |
| | ACGAGGCAUUAGCACUCCUAUAAGGUGUGAAAGmUsmCsmUsG | |
| sgRNA_369 | mGsmUsmUsCUGUCUUUUGGUCAGGACAACCGUCUAGCUAUAAG | 487 |
| | UGCUGCAGGGUGUGAGAAACUCCUAUUGCUGGACGAUGUCUCUU | |
| | ACGAGGCAUUAGCACUGAGCCCAUGCAGCUCUCmCsmAsmGsA | |
| sgRNA_370 | mGsmUsmUsCUGUCUUUUGGUCAGGACAACCGUCUAGCUAUAAG | 488 |
| | UGCUGCAGGGUGUGAGAAACUCCUAUUGCUGGACGAUGUCUCUU | |
| | ACGAGGCAUUAGCACCUCCUCAGUUGUGAGCCCmAsmUsmGsC | |
| sgRNA_371 | mGsmUsmUsCUGUCUUUUGGUCAGGACAACCGUCUAGCUAUAAG | 489 |
| | UGCUGCAGGGUGUGAGAAACUCCUAUUGCUGGACGAUGUCUCUU | |
| | ACGAGGCAUUAGCACGUAGAAGGGAUAUACAAAmGsmUsmGsG | |
| sgRNA_372 | mGsmUsmUsCUGUCUUUUGGUCAGGACAACCGUCUAGCUAUAAG | 490 |
| | UGCUGCAGGGUGUGAGAAACUCCUAUUGCUGGACGAUGUCUCUU | |
| | ACGAGGCAUUAGCACCCACUUUGUAUAUCCCUUmCsmUsmAsC | |
| sgRNA_373 | mGsmUsmUsCUGUCUUUUGGUCAGGACAACCGUCUAGCUAUAAG | 491 |
| | UGCUGCAGGGUGUGAGAAACUCCUAUUGCUGGACGAUGUCUCUU | |
| | ACGAGGCAUUAGCACGGUGUCUAUUUCCACUUUmGsmUsmAsU | |
| sgRNA_374 | mGsmUsmUsCUGUCUUUUGGUCAGGACAACCGUCUAGCUAUAAG | 492 |
| | UGCUGCAGGGUGUGAGAAACUCCUAUUGCUGGACGAUGUCUCUU | |
| | ACGAGGCAUUAGCACCAUGAGCAUGCAGAGGUGmAsmGsmUsA | |
| gRNA1594 | mCsmAsmAsCUUACCCAGAGGCAAAUGUUUUAGAGCUAGAAAUA | 493 |
| | GCAAGUUAAAAUAAGGCUAGUCCGUUAUCAACUUGAAAAAGUGG | |
| | CACCGAGUCGGUGCUsmUsmUsmU | |
| gRNA1595 | mAsmAsmUsGGCUCCCAGGUGUCAUCGUUUUAGAGCUAGAAAUA | 494 |
| | GCAAGUUAAAAUAAGGCUAGUCCGUUAUCAACUUGAAAAAGUGG | |
| | CACCGAGUCGGUGCUsmUsmUsmU | |
| gRNA1596 | mGsmGsmCsUCCCAGGUGUCAUCAGCGUUUUAGAGCUAGAAAUA | 495 |
| | GCAAGUUAAAAUAAGGCUAGUCCGUUAUCAACUUGAAAAAGUGG | |
| | CACCGAGUCGGUGCUsmUsmUsmU | |
| gRNA1597 | mCsmUsmCsUCAUAGGUGGUAUUCACGUUUUAGAGCUAGAAAUA | 496 |
| | GCAAGUUAAAAUAAGGCUAGUCCGUUAUCAACUUGAAAAAGUGG | |
| | CACCGAGUCGGUGCUsmUsmUsmU | |
| gRNA1598 | mUsmAsmUsAGGAAAACCAGUGAGUCGUUUUAGAGCUAGAAAUA | 497 |
| | GCAAGUUAAAAUAAGGCUAGUCCGUUAUCAACUUGAAAAAGUGG | |
| | CACCGAGUCGGUGCUsmUsmUsmU | |
| gRNA1599 | mUsmAsmCsUCACCUCUGCAUGCUCAGUUUUAGAGCUAGAAAUA | 480 |
| | GCAAGUUAAAAUAAGGCUAGUCCGUUAUCAACUUGAAAAAGUGG | |
| | CACCGAGUCGGUGCUsmUsmUsmU | |
| gRNA1600 | mGsmCsmAsACUUACCCAGAGGCAAAGUUUUAGAGCUAGAAAUA | 499 |
| | GCAAGUUAAAAUAAGGCUAGUCCGUUAUCAACUUGAAAAAGUGG | |
| | CACCGAGUCGGUGCUsmUsmUsmU | |
| gRNA1601 | mUsmCsmUsGUAUACUCACCUCUGCAGUUUUAGAGCUAGAAAUA | 500 |
| | GCAAGUUAAAAUAAGGCUAGUCCGUUAUCAACUUGAAAAAGUGG | |
| | CACCGAGUCGGUGCUsmUsmUsmU | |
| gRNA1602 | mGsmAsmAsACACUCACCGUAGGGCCGUUUUAGAGCUAGAAAUA | 501 |
| | GCAAGUUAAAAUAAGGCUAGUCCGUUAUCAACUUGAAAAAGUGG | |
| | CACCGAGUCGGUGCUsmUsmUsmU | |
| gRNA1603 | mCsmUsmCsUACACCCAGGGCACCGGGUUUUAGAGCUAGAAAUA | 502 |
| | GCAAGUUAAAAUAAGGCUAGUCCGUUAUCAACUUGAAAAAGUGG | |
| | CACCGAGUCGGUGCUsmUsmUsmU | |
| gRNA1604 | mAsmCsmAsCCUUAUAGGAAAACCAGGUUUUAGAGCUAGAAAUA | 503 |
| | GCAAGUUAAAAUAAGGCUAGUCCGUUAUCAACUUGAAAAAGUGG | |
| | CACCGAGUCGGUGCUsmUsmUsmU | |
| gRNA1605 | mAsmUsmAsGGAAAACCAGUGAGUCUGUUUUAGAGCUAGAAAUA | 504 |
| | GCAAGUUAAAAUAAGGCUAGUCCGUUAUCAACUUGAAAAAGUGG | |
| | CACCGAGUCGGUGCUsmUsmUsmU | |
| gRNA1606 | mAsmCsmUsCACCUCUGCAUGCUCAUGUUUUAGAGCUAGAAAUA | 481 |
| | GCAAGUUAAAAUAAGGCUAGUCCGUUAUCAACUUGAAAAAGUGG | |
| | CACCGAGUCGGUGCUsmUsmUsmU | |
| gRNA1607 | mCsmUsmCsACCGUAGGGCCAGCCUCGUUUUAGAGCUAGAAAUA | 506 |
| | GCAAGUUAAAAUAAGGCUAGUCCGUUAUCAACUUGAAAAAGUGG | |
| | CACCGAGUCGGUGCUsmUsmUsmU | |
| gRNA1746 | mAsmAsmCsCUGCUGAUUCUGAUUAUGUUUUAGAGCUAGAAAUA | 507 |
| | GCAAGUUAAAAUAAGGCUAGUCCGUUAUCAACUUGAAAAAGUGG | |
| | CACCGAGUCGGUGCUsmUsmUsmU | |
| gRNA1747 | mAsmAsmGsAGAGAAUAAGUAACCCAUGUUUUAGUACUCUGUAA | 508 |
| | UGAAAAUUACAGAAUCUACUAAAACAAGGCAAAAUGCCGUGUUU | |
| | AUCUCGUCAACUUGUUGGCGAGAUsmUsmUsmU | |
| gRNA1748 | mAsmAsmGsCAGCCUAGCUCAGGAGAAGUUUUAGUACUCUGUAA | 509 |
| | UGAAAAUUACAGAAUCUACUAAAACAAGGCAAAAUGCCGUGUUU | |
| | AUCUCGUCAACUUGUUGGCGAGAUsmUsmUsmU | |
| gRNA1749 | mAsmAsmGsUCCACUCAUUCUUGGCAGUUUUAGAGCUAGAAAUA | 510 |
| | GCAAGUUAAAAUAAGGCUAGUCCGUUAUCAACUUGAAAAAGUGG | |
| | CACCGAGUCGGUGCUsmUsmUsmU | |
| gRNA1750 | mAsmCsmGsAUGAGAAGCCAUCCUGCCGUUUUAGUACUCUGUAA | 511 |
| | UGAAAAUUACAGAAUCUACUAAAACAAGGCAAAAUGCCGUGUUU | |
| | AUCUCGUCAACUUGUUGGCGAGAUsmUsmUsmU | |
| gRNA1751 | mAsmGsmAsCAAGGUUCAUAUUUGUAGUUUUAGAGCUAGAAAUA | 512 |
| | GCAAGUUAAAAUAAGGCUAGUCCGUUAUCAACUUGAAAAAGUGG | |
| | CACCGAGUCGGUGCUsmUsmUsmU | |
| gRNA1752 | mAsmGsmGsCUGGGAGCAGCCAUCACGUUUUAGAGCUAGAAAUA | 513 |
| | GCAAGUUAAAAUAAGGCUAGUCCGUUAUCAACUUGAAAAAGUGG | |
| | CACCGAGUCGGUGCUsmUsmUsmU | |
| gRNA1753 | mAsmUsmAsAGUAACCCAUACAAAUAGUUUUAGAGCUAGAAAUA | 514 |
| | GCAAGUUAAAAUAAGGCUAGUCCGUUAUCAACUUGAAAAAGUGG | |
| | CACCGAGUCGGUGCUsmUsmUsmU | |
| gRNA1754 | mAsmUsmAsCUCACUUCUCCUGAGCUGUUUUAGAGCUAGAAAUA | 515 |
| | GCAAGUUAAAAUAAGGCUAGUCCGUUAUCAACUUGAAAAAGUGG | |
| | CACCGAGUCGGUGCUsmUsmUsmU | |
| gRNA1755 | mAsmUsmUsAUUGACUUAGUCAACAAGUUUUAGAGCUAGAAAUA | 516 |
| | GCAAGUUAAAAUAAGGCUAGUCCGUUAUCAACUUGAAAAAGUGG | |
| | CACCGAGUCGGUGCUsmUsmUsmU | |
| gRNA1756 | mCsmAsmAsAUAUGAACCUUGUCUAGGUUUUAGAGCUAGAAAUA | 517 |
| | GCAAGUUAAAAUAAGGCUAGUCCGUUAUCAACUUGAAAAAGUGG | |
| | CACCGAGUCGGUGCUsmUsmUsmU | |
| gRNA1757 | mCsmAsmGsAAGUCCACUCAUUCUUGGGUUUUAGUACUCUGUAA | 518 |
| | UGAAAAUUACAGAAUCUACUAAAACAAGGCAAAAUGCCGUGUUU | |
| | AUCUCGUCAACUUGUUGGCGAGAUsmUsmUsmU | |
| gRNA1758 | mCsmAsmGsGCUGGGAGCAGCCAUCACGUUUUAGUACUCUGUAA | 519 |
| | UGAAAAUUACAGAAUCUACUAAAACAAGGCAAAAUGCCGUGUUU | |
| | AUCUCGUCAACUUGUUGGCGAGAUsmUsmUsmU | |
| gRNA1759 | mCsmCsmAsUCCUGCCAAGAAUGAGUGUUUUAGAGCUAGAAAUA | 520 |
| | GCAAGUUAAAAUAAGGCUAGUCCGUUAUCAACUUGAAAAAGUGG | |
| | CACCGAGUCGGUGCUsmUsmUsmU | |
| gRNA1760 | mCsmCsmUsGCUGAUUCUGAUUAUUGAGUUUUAGUACUCUGUAA | 521 |
| | UGAAAAUUACAGAAUCUACUAAAACAAGGCAAAAUGCCGUGUUU | |
| | AUCUCGUCAACUUGUUGGCGAGAUsmUsmUsmU | |
| gRNA1761 | mCsmGsmAsUGCUCUAAUCUCUCUAGAGUUUUAGUACUCUGUAA | 522 |
| | UGAAAAUUACAGAAUCUACUAAAACAAGGCAAAAUGCCGUGUUU | |
| | AUCUCGUCAACUUGUUGGCGAGAUsmUsmUsmU | |
| gRNA1762 | mCsmUsmAsAGUCAAUAAUCAGAAUCAGUUUUAGUACUCUGUAA | 523 |
| | UGAAAAUUACAGAAUCUACUAAAACAAGGCAAAAUGCCGUGUUU | |
| | AUCUCGUCAACUUGUUGGCGAGAUsmUsmUsmU | |
| gRNA1763 | mCsmUsmAsGACAAGGUUCAUAUUUGUGUUUUAGUACUCUGUAA | 524 |
| | UGAAAAUUACAGAAUCUACUAAAACAAGGCAAAAUGCCGUGUUU | |
| | AUCUCGUCAACUUGUUGGCGAGAUsmUsmUsmU | |
| gRNA1764 | mGsmAsmAsCCUUGUCUAGAGAGAUUGUUUUAGAGCUAGAAAUA | 525 |
| | GCAAGUUAAAAUAAGGCUAGUCCGUUAUCAACUUGAAAAAGUGG | |
| | CACCGAGUCGGUGCUsmUsmUsmU | |
| gRNA1765 | mGsmAsmAsGUCCACUCAUUCUUGGCGUUUUAGAGCUAGAAAUA | 526 |
| | GCAAGUUAAAAUAAGGCUAGUCCGUUAUCAACUUGAAAAAGUGG | |
| | CACCGAGUCGGUGCUsmUsmUsmU | |
| gRNA1766 | mGsmAsmAsUCAGCAGGUUUGCAGUCGUUUUAGAGCUAGAAAUA | 527 |
| | GCAAGUUAAAAUAAGGCUAGUCCGUUAUCAACUUGAAAAAGUGG | |
| | CACCGAGUCGGUGCUsmUsmUsmU | |
| gRNA1767 | mGsmAsmAsUGAGUGGACUUCUGUGAGUUUUAGAGCUAGAAAUA | 528 |
| | GCAAGUUAAAAUAAGGCUAGUCCGUUAUCAACUUGAAAAAGUGG | |
| | CACCGAGUCGGUGCUsmUsmUsmU | |
| gRNA1768 | mGsmAsmCsUGCAAACCUGCUGAUUCGUUUUAGAGCUAGAAAUA | 529 |
| | GCAAGUUAAAAUAAGGCUAGUCCGUUAUCAACUUGAAAAAGUGG | |
| | CACCGAGUCGGUGCUsmUsmUsmU | |
| gRNA1769 | mGsmAsmCsUUAGUCAACAAAGAGAGAGUUUUAGUACUCUGUAA | 530 |
| | UGAAAAUUACAGAAUCUACUAAAACAAGGCAAAAUGCCGUGUUU | |
| | AUCUCGUCAACUUGUUGGCGAGAUsmUsmUsmU | |
| gRNA1770 | mGsmAsmUsAAGCAGCCUAGCUCAGGGUUUUAGAGCUAGAAAUA | 531 |
| | GCAAGUUAAAAUAAGGCUAGUCCGUUAUCAACUUGAAAAAGUGG | |
| | CACCGAGUCGGUGCUsmUsmUsmU | |
| gRNA1771 | mGsmAsmUsGAGAAGCCAUCCUGCCAGUUUUAGAGCUAGAAAUA | 532 |
| | GCAAGUUAAAAUAAGGCUAGUCCGUUAUCAACUUGAAAAAGUGG | |
| | CACCGAGUCGGUGCUsmUsmUsmU | |
| gRNA1772 | mGsmCsmCsAUCCUGCCAAGAAUGAGGUUUUAGAGCUAGAAAUA | 477 |
| | GCAAGUUAAAAUAAGGCUAGUCCGUUAUCAACUUGAAAAAGUGG | |
| | CACCGAGUCGGUGCUsmUsmUsmU | |
| gRNA1773 | mGsmCsmUsUUUAUACUCACUUCUCCGUUUUAGAGCUAGAAAUA | 534 |
| | GCAAGUUAAAAUAAGGCUAGUCCGUUAUCAACUUGAAAAAGUGG | |
| | CACCGAGUCGGUGCUsmUsmUsmU | |
| gRNA1774 | mGsmGsmASUAAGCAGCCUAGCUCAGGGUUUUAGUACUCUGUAA | 535 |
| | UGAAAAUUACAGAAUCUACUAAAACAAGGCAAAAUGCCGUGUUU | |
| | AUCUCGUCAACUUGUUGGCGAGAUsmUsmUsmU | |
| gRNA1775 | mGsmUsmCsUAGAGAGAUUAGAGCAUGUUUUAGAGCUAGAAAUA | 536 |
| | GCAAGUUAAAAUAAGGCUAGUCCGUUAUCAACUUGAAAAAGUGG | |
| | CACCGAGUCGGUGCUsmUsmUsmU | |
| gRNA1776 | mGsmUsmGsAUGGCUGCUCCCAGCCUGUUUUAGAGCUAGAAAUA | 537 |
| | GCAAGUUAAAAUAAGGCUAGUCCGUUAUCAACUUGAAAAAGUGG | |
| | CACCGAGUCGGUGCUsmUsmUsmU | |
| gRNA1777 | mUsmAsmCsUUAUUCUCUCUUUGUUGAGUUUUAGUACUCUGUAA | 538 |
| | UGAAAAUUACAGAAUCUACUAAAACAAGGCAAAAUGCCGUGUUU | |
| | AUCUCGUCAACUUGUUGGCGAGAUsmUsmUsmU | |
| gRNA1778 | mUsmAsmUsUCUCUCUUUGUUGACUAAGUUUUAGUACUCUGUAA | 539 |
| | UGAAAAUUACAGAAUCUACUAAAACAAGGCAAAAUGCCGUGUUU | |
| | AUCUCGUCAACUUGUUGGCGAGAUsmUsmUsmU | |
| gRNA1779 | mUsmAsmUsUGACUUAGUCAACAAAGGUUUUAGAGCUAGAAAUA | 540 |
| | GCAAGUUAAAAUAAGGCUAGUCCGUUAUCAACUUGAAAAAGUGG | |
| | CACCGAGUCGGUGCUsmUsmUsmU | |
| gRNA1780 | mUsmAsmUsUGACUUAGUCAACAAAGAGUUUUAGUACUCUGUAA | 541 |
| | UGAAAAUUACAGAAUCUACUAAAACAAGGCAAAAUGCCGUGUUU | |
| | AUCUCGUCAACUUGUUGGCGAGAUsmUsmUsmU | |
| gRNA1781 | mUsmCsmAsGAAUCAGCAGGUUUGCAGGUUUUAGUACUCUGUAA | 542 |
| | UGAAAAUUACAGAAUCUACUAAAACAAGGCAAAAUGCCGUGUUU | |
| | AUCUCGUCAACUUGUUGGCGAGAUsmUsmUsmU | |
| gRNA1782 | mUsmCsmCsACUCAUUCUUGGCAGGAGUUUUAGAGCUAGAAAUA | 543 |
| | GCAAGUUAAAAUAAGGCUAGUCCGUUAUCAACUUGAAAAAGUGG | |
| | CACCGAGUCGGUGCUsmUsmUsmU | |
| gRNA1783 | mUsmCsmUsCUCUUUGUUGACUAAGUCGUUUUAGUACUCUGUAA | 544 |
| | UGAAAAUUACAGAAUCUACUAAAACAAGGCAAAAUGCCGUGUUU | |
| | AUCUCGUCAACUUGUUGGCGAGAUsmUsmUsmU | |
| gRNA1784 | mUsmGsmAsGAAGCCAUCCUGCCAAGAGUUUUAGUACUCUGUAA | 545 |
| | UGAAAAUUACAGAAUCUACUAAAACAAGGCAAAAUGCCGUGUUU | |
| | AUCUCGUCAACUUGUUGGCGAGAUsmUsmUsmU | |
| gRNA1785 | mUsmGsmAsGCUAGGCUGCUUAUCCCUGUUUUAGUACUCUGUAA | 546 |
| | UGAAAAUUACAGAAUCUACUAAAACAAGGCAAAAUGCCGUGUUU | |
| | AUCUCGUCAACUUGUUGGCGAGAUsmUsmUsmU | |
| gRNA1786 | mUsmGsmAsGUAUAAAAGCCCCAGGCGUUUUAGAGCUAGAAAUA | 547 |
| | GCAAGUUAAAAUAAGGCUAGUCCGUUAUCAACUUGAAAAAGUGG | |
| | CACCGAGUCGGUGCUsmUsmUsmU | |
| gRNA1787 | mUsmGsmAsUGGCUGCUCCCAGCCUGGUUUUAGAGCUAGAAAUA | 548 |
| | GCAAGUUAAAAUAAGGCUAGUCCGUUAUCAACUUGAAAAAGUGG | |
| | CACCGAGUCGGUGCUsmUsmUsmU | |
| gRNA1788 | mUsmGsmCsCAAGAAUGAGUGGACUUCGUUUUAGUACUCUGUAA | 549 |
| | UGAAAAUUACAGAAUCUACUAAAACAAGGCAAAAUGCCGUGUUU | |
| | AUCUCGUCAACUUGUUGGCGAGAUsmUsmUsmU | |
| gRNA1789 | mUsmGsmCsCAAUCUGACUGCAAACCUGUUUUAGUACUCUGUAA | 550 |
| | UGAAAAUUACAGAAUCUACUAAAACAAGGCAAAAUGCCGUGUUU | |
| | AUCUCGUCAACUUGUUGGCGAGAUsmUsmUsmU | |
| gRNA1790 | mUsmGsmUsUGACUAAGUCAAUAAUCGUUUUAGAGCUAGAAAUA | 551 |
| | GCAAGUUAAAAUAAGGCUAGUCCGUUAUCAACUUGAAAAAGUGG | |
| | CACCGAGUCGGUGCUsmUsmUsmU | |
| gRNA1791 | mUsmUsmGsACUUAGUCAACAAAGAGGUUUUAGAGCUAGAAAUA | 552 |
| | GCAAGUUAAAAUAAGGCUAGUCCGUUAUCAACUUGAAAAAGUGG | |
| | CACCGAGUCGGUGCUsmUsmUsmU | |
| gRNA1792 | mUsmUsmUsGUUGACUAAGUCAAUAAUGUUUUAGUACUCUGUAA | 553 |
| | UGAAAAUUACAGAAUCUACUAAAACAAGGCAAAAUGCCGUGUUU | |
| | AUCUCGUCAACUUGUUGGCGAGAUsmUsmUsmU | |
| gRNA-#1 | mAsmAsmAsAGCCCCAGGCUGGGAGCGUUUUAGAGCUAGAAAUA | 554 |
| | GCAAGUUAAAAUAAGGCUAGUCCGUUAUCAACUUGAAAAAGUGG | |
| | CACCGAGUCGGUGCUsmUsmUsmU | |
| gRNA-#2 | mAsmAsmGsUGAGUAUAAAAGCCCCAGUUUUAGAGCUAGAAAUA | 555 |
| | GCAAGUUAAAAUAAGGCUAGUCCGUUAUCAACUUGAAAAAGUGG | |
| | CACCGAGUCGGUGCUsmUsmUsmU | |
| gRNA-#3 | mAsmAsmUsAAUCAGAAUCAGCAGGUUGUUUUAGUACUCUGUAA | 556 |
| | UGAAAAUUACAGAAUCUACUAAAACAAGGCAAAAUGCCGUGUUU | |
| | AUCUCGUCAACUUGUUGGCGAGAUsmUsmUsmU | |
| gRNA-#4 | mAsmAsmUsAUGAACCUUGUCUAGAGGUUUUAGAGCUAGAAAUA | 557 |
| | GCAAGUUAAAAUAAGGCUAGUCCGUUAUCAACUUGAAAAAGUGG | |
| | CACCGAGUCGGUGCUsmUsmUsmU | |
| gRNA-#5 | mAsmAsmUsGAGUGGACUUCUGUGAUGUUUUAGAGCUAGAAAUA | 558 |
| | GCAAGUUAAAAUAAGGCUAGUCCGUUAUCAACUUGAAAAAGUGG | |
| | CACCGAGUCGGUGCUsmUsmUsmU | |
| gRNA-#6 | mAsmCsmAsAAUAUGAACCUUGUCUAGGUUUUAGUACUCUGUAA | 559 |
| | UGAAAAUUACAGAAUCUACUAAAACAAGGCAAAAUGCCGUGUUU | |
| | AUCUCGUCAACUUGUUGGCGAGAUsmUsmUsmU | |
| gRNA-#7 | mAsmCsmAsGAAGUCCACUCAUUCUUGUUUUAGAGCUAGAAAUA | 560 |
| | GCAAGUUAAAAUAAGGCUAGUCCGUUAUCAACUUGAAAAAGUGG | |
| | CACCGAGUCGGUGCUsmUsmUsmU | |
| gRNA-#8 | mAsmCsmCsUUGUCUAGAGAGAUUAGGUUUUAGAGCUAGAAAUA | 561 |
| | GCAAGUUAAAAUAAGGCUAGUCCGUUAUCAACUUGAAAAAGUGG | |
| | CACCGAGUCGGUGCUsmUsmUsmU | |
| gRNA-#9 | mAsmGsmAsAGCCAUCCUGCCAAGAAGUUUUAGAGCUAGAAAUA | 562 |
| | GCAAGUUAAAAUAAGGCUAGUCCGUUAUCAACUUGAAAAAGUGG | |
| | CACCGAGUCGGUGCUsmUsmUsmU | |
| gRNA-#10 | mAsmGsmCsAGGUUUGCAGUCAGAUUGUUUUAGAGCUAGAAAUA | 563 |
| | GCAAGUUAAAAUAAGGCUAGUCCGUUAUCAACUUGAAAAAGUGG | |
| | CACCGAGUCGGUGCUsmUsmUsmU | |
| gRNA-#11 | mAsmGsmGsGAUAAGCAGCCUAGCUCGUUUUAGAGCUAGAAAUA | 564 |
| | GCAAGUUAAAAUAAGGCUAGUCCGUUAUCAACUUGAAAAAGUGG | |
| | CACCGAGUCGGUGCUsmUsmUsmU | |
| gRNA-#12 | mAsmGsmGsUUUGCAGUCAGAUUGGCGUUUUAGAGCUAGAAAUA | 565 |
| | GCAAGUUAAAAUAAGGCUAGUCCGUUAUCAACUUGAAAAAGUGG | |
| | CACCGAGUCGGUGCUsmUsmUsmU | |
| gRNA-#13 | mAsmGsmUsAUAAAAGCCCCAGGCUGGUUUUAGAGCUAGAAAUA | 566 |
| | GCAAGUUAAAAUAAGGCUAGUCCGUUAUCAACUUGAAAAAGUGG | |
| | CACCGAGUCGGUGCUsmUsmUsmU | |
| gRNA-#14 | mAsmGsmUsCAAUAAUCAGAAUCAGCGUUUUAGAGCUAGAAAUA | 567 |
| | GCAAGUUAAAAUAAGGCUAGUCCGUUAUCAACUUGAAAAAGUGG | |
| | CACCGAGUCGGUGCUsmUsmUsmU | |
| gRNA-#15 | mAsmUsmAsAUCAGAAUCAGCAGGUUGUUUUAGAGCUAGAAAUA | 568 |
| | GCAAGUUAAAAUAAGGCUAGUCCGUUAUCAACUUGAAAAAGUGG | |
| | CACCGAGUCGGUGCUsmUsmUsmU | |
| gRNA-#16 | mGsmUsmUsCUGUCUUUUGGUCAGGACAACCGUCUAGCUAUAAG | 569 |
| | UGCUGCAGGGUGUGAGAAACUCCUAUUGCUGGACGAUGUCUCUU | |
| | ACGAGGCAUUAGCACUUGACUUAGUCAACAAAGAsmGsmAsmG | |
| gRNA-#17 | mGsmUsmUsCUGUCUUUUGGUCAGGACAACCGUCUAGCUAUAAG | 570 |
| | UGCUGCAGGGUGUGAGAAACUCCUAUUGCUGGACGAUGUCUCUU | |
| | ACGAGGCAUUAGCACUCUCUUUGUUGACUAAGUCsmAsmAsmU | |
| gRNA-#18 | mGsmUsmUsCUGUCUUUUGGUCAGGACAACCGUCUAGCUAUAAG | 571 |
| | UGCUGCAGGGUGUGAGAAACUCCUAUUGCUGGACGAUGUCUCUU | |
| | ACGAGGCAUUAGCACUGAUUAUUGACUUAGUCAAsmCsmAsmA | |
| gRNA-#19 | mGsmUsmUsCUGUCUUUUGGUCAGGACAACCGUCUAGCUAUAAG | 485 |
| | UGCUGCAGGGUGUGAGAAACUCCUAUUGCUGGACGAUGUCUCUU | |
| | ACGAGGCAUUAGCACUUGGCAGGAUGGCUUCUCAsmUsmCsmG | |
| gRNA-#20 | mGsmUsmUsCUGUCUUUUGGUCAGGACAACCGUCUAGCUAUAAG | 573 |
| | UGCUGCAGGGUGUGAGAAACUCCUAUUGCUGGACGAUGUCUCUU | |
| | ACGAGGCAUUAGCACACUUAGUCAACAAAGAGAGsmAsmAsmU | |
| gRNA-#21 | mGsmUsmUsCUGUCUUUUGGUCAGGACAACCGUCUAGCUAUAAG | 574 |
| | UGCUGCAGGGUGUGAGAAACUCCUAUUGCUGGACGAUGUCUCUU | |
| | ACGAGGCAUUAGCACGCAGGGAUAAGCAGCCUAGsmCsmUsmC | |
| gRNA-#22 | mGsmUsmUsCUGUCUUUUGGUCAGGACAACCGUCUAGCUAUAAG | 575 |
| | UGCUGCAGGGUGUGAGAAACUCCUAUUGCUGGACGAUGUCUCUU | |
| | ACGAGGCAUUAGCACGUAUGGGUUACUUAUUCUCsmUsmCsmU | |
| gRNA-#23 | mCsmAsmAsGAAUGAGUGGACUUCUGGUUUUAGAGCUAGAAAUA | 576 |
| | GCAAGUUAAAAUAAGGCUAGUCCGUUAUCAACUUGAAAAAGUGG | |
| | CACCGAGUCGGUGCUsmUsmUsmU | |
| gRNA-#24 | mCsmAsmAsUCUGACUGCAAACCUGCGUUUUAGAGCUAGAAAUA | 577 |
| | GCAAGUUAAAAUAAGGCUAGUCCGUUAUCAACUUGAAAAAGUGG | |
| | CACCGAGUCGGUGCUsmUsmUsmU | |
| gRNA-#25 | mCsmAsmCsAGAAGUCCACUCAUUCUGUUUUAGAGCUAGAAAUA | 578 |
| | GCAAGUUAAAAUAAGGCUAGUCCGUUAUCAACUUGAAAAAGUGG | |
| | CACCGAGUCGGUGCUsmUsmUsmU | |
| gRNA-#26 | mCsmAsmGsACGAUGAGAAGCCAUCCGUUUUAGAGCUAGAAAUA | 579 |
| | GCAAGUUAAAAUAAGGCUAGUCCGUUAUCAACUUGAAAAAGUGG | |
| | CACCGAGUCGGUGCUsmUsmUsmU | |
| gRNA-#27 | mCsmAsmGsCAGGUUUGCAGUCAGAUGUUUUAGAGCUAGAAAUA | 580 |
| | GCAAGUUAAAAUAAGGCUAGUCCGUUAUCAACUUGAAAAAGUGG | |
| | CACCGAGUCGGUGCUsmUsmUsmU | |
| gRNA-#28 | mCsmAsmGsGAUGGCUUCUCAUCGUCGUUUUAGAGCUAGAAAUA | 581 |
| | GCAAGUUAAAAUAAGGCUAGUCCGUUAUCAACUUGAAAAAGUGG | |
| | CACCGAGUCGGUGCUsmUsmUsmU | |
| gRNA-#29 | mCsmAsmGsGUUUGCAGUCAGAUUGGCGUUUUAGUACUCUGUAA | 582 |
| | UGAAAAUUACAGAAUCUACUAAAACAAGGCAAAAUGCCGUGUUU | |
| | AUCUCGUCAACUUGUUGGCGAGAUsmUsmUsmU | |
| gRNA-#30 | mCsmAsmGsUCAGAUUGGCAGGGAUAGUUUUAGAGCUAGAAAUA | 583 |
| | GCAAGUUAAAAUAAGGCUAGUCCGUUAUCAACUUGAAAAAGUGG | |
| | CACCGAGUCGGUGCUsmUsmUsmU | |
| gRNA-#31 | mCsmCsmAsCUCAUUCUUGGCAGGAUGUUUUAGAGCUAGAAAUA | 584 |
| | GCAAGUUAAAAUAAGGCUAGUCCGUUAUCAACUUGAAAAAGUGG | |
| | CACCGAGUCGGUGCUsmUsmUsmU | |
| gRNA-#32 | mCsmUsmAsAGUCAAUAAUCAGAAUCGUUUUAGAGCUAGAAAUA | 585 |
| | GCAAGUUAAAAUAAGGCUAGUCCGUUAUCAACUUGAAAAAGUGG | |
| | CACCGAGUCGGUGCUsmUsmUsmU | |
| gRNA-#33 | mGsmUsmUsCUGUCUUUUGGUCAGGACAACCGUCUAGCUAUAAG | 586 |
| | UGCUGCAGGGUGUGAGAAACUCCUAUUGCUGGACGAUGUCUCUU | |
| | ACGAGGCAUUAGCACGUCAACAAAGAGAGAAUAAsmGsmUsmA | |
| gRNA-#34 | mCsmUsmUsAUCCCUGCCAAUCUGACGUUUUAGAGCUAGAAAUA | 587 |
| | GCAAGUUAAAAUAAGGCUAGUCCGUUAUCAACUUGAAAAAGUGG | |
| | CACCGAGUCGGUGCUsmUsmUsmU | |
| gRNA-#35 | mGsmUsmUsCUGUCUUUUGGUCAGGACAACCGUCUAGCUAUAAG | 588 |
| | UGCUGCAGGGUGUGAGAAACUCCUAUUGCUGGACGAUGUCUCUU | |
| | ACGAGGCAUUAGCACUCCCUGCCAAUCUGACUGCsmAsmAsmA | |
| gRNA-#36 | mGsmUsmUsCUGUCUUUUGGUCAGGACAACCGUCUAGCUAUAAG | 589 |
| | UGCUGCAGGGUGUGAGAAACUCCUAUUGCUGGACGAUGUCUCUU | |
| | ACGAGGCAUUAGCACUUCUCUCUUUGUUGACUAAsmGsmUsmC | |
| gRNA-#37 | mGsmUsmUsCUGUCUUUUGGUCAGGACAACCGUCUAGCUAUAAG | 590 |
| | UGCUGCAGGGUGUGAGAAACUCCUAUUGCUGGACGAUGUCUCUU | |
| | ACGAGGCAUUAGCACUCCUGAGCUAGGCUGCUUAsmUsmCsmC | |
| gRNA-#38 | mCsmUsmUsCUGUGAUGGCUGCUCCCGUUUUAGAGCUAGAAAUA | 591 |
| | GCAAGUUAAAAUAAGGCUAGUCCGUUAUCAACUUGAAAAAGUGG | |
| | CACCGAGUCGGUGCUsmUsmUsmU | |
| gRNA-#39 | mGsmUsmUsCUGUCUUUUGGUCAGGACAACCGUCUAGCUAUAAG | 592 |
| | UGCUGCAGGGUGUGAGAAACUCCUAUUGCUGGACGAUGUCUCUU | |
| | ACGAGGCAUUAGCACUGUGAUGGCUGCUCCCAGCsmCsmUsmG | |
| gRNA-#40 | mGsmUsmUsCUGUCUUUUGGUCAGGACAACCGUCUAGCUAUAAG | 593 |
| | UGCUGCAGGGUGUGAGAAACUCCUAUUGCUGGACGAUGUCUCUU | |
| | ACGAGGCAUUAGCACGCAGGAUGGCUUCUCAUCGsmUsmCsmU | |
| gRNA-#41 | mGsmUsmUsCUGUCUUUUGGUCAGGACAACCGUCUAGCUAUAAG | 594 |
| | UGCUGCAGGGUGUGAGAAACUCCUAUUGCUGGACGAUGUCUCUU | |
| | ACGAGGCAUUAGCACUCUAGAGAGAUUAGAGCAUsmCsmGsmG | |
| gRNA-#42 | mGsmUsmUsCUGUCUUUUGGUCAGGACAACCGUCUAGCUAUAAG | 595 |
| | UGCUGCAGGGUGUGAGAAACUCCUAUUGCUGGACGAUGUCUCUU | |
| | ACGAGGCAUUAGCACGUUGACUAAGUCAAUAAUCsmAsmGsmA | |
| gRNA-#43 | mGsmUsmUsCUGUCUUUUGGUCAGGACAACCGUCUAGCUAUAAG | 596 |
| | UGCUGCAGGGUGUGAGAAACUCCUAUUGCUGGACGAUGUCUCUU | |
| | ACGAGGCAUUAGCACUAUACUCACUUCUCCUGAGsmCsmUsmA | |
| gRNA-#44 | mGsmAsmAsGUGAGUAUAAAAGCCCCGUUUUAGAGCUAGAAAUA | 597 |
| | GCAAGUUAAAAUAAGGCUAGUCCGUUAUCAACUUGAAAAAGUGG | |
| | CACCGAGUCGGUGCUsmUsmUsmU | |
| gRNA-#45 | mGsmAsmCsAAGGUUCAUAUUUGUAUGUUUUAGAGCUAGAAAUA | 598 |
| | GCAAGUUAAAAUAAGGCUAGUCCGUUAUCAACUUGAAAAAGUGG | |
| | CACCGAGUCGGUGCUsmUsmUsmU | |
| gRNA-#46 | mGsmAsmGsUAUAAAAGCCCCAGGCUGUUUUAGAGCUAGAAAUA | 599 |
| | GCAAGUUAAAAUAAGGCUAGUCCGUUAUCAACUUGAAAAAGUGG | |
| | CACCGAGUCGGUGCUsmUsmUsmU | |
| gRNA-#47 | mGsmAsmGsUGGACUUCUGUGAUGGCGUUUUAGAGCUAGAAAUA | 600 |
| | GCAAGUUAAAAUAAGGCUAGUCCGUUAUCAACUUGAAAAAGUGG | |
| | CACCGAGUCGGUGCUsmUsmUsmU | |
| gRNA-#48 | mGsmAsmUsGGCUGCUCCCAGCCUGGGUUUUAGAGCUAGAAAUA | 601 |
| | GCAAGUUAAAAUAAGGCUAGUCCGUUAUCAACUUGAAAAAGUGG | |
| | CACCGAGUCGGUGCUsmUsmUsmU | |
| gRNA-#49 | mGsmCsmAsGCCUAGCUCAGGAGAAGGUUUUAGAGCUAGAAAUA | 602 |
| | GCAAGUUAAAAUAAGGCUAGUCCGUUAUCAACUUGAAAAAGUGG | |
| | CACCGAGUCGGUGCUsmUsmUsmU | |
| gRNA-#50 | mGsmCsmUsGCUUAUCCCUGCCAAUCGUUUUAGAGCUAGAAAUA | 603 |
| | GCAAGUUAAAAUAAGGCUAGUCCGUUAUCAACUUGAAAAAGUGG | |
| | CACCGAGUCGGUGCUsmUsmUsmU | |
| gRNA-#51 | mGsmGsmGsAUAAGCAGCCUAGCUCAGUUUUAGAGCUAGAAAUA | 604 |
| | GCAAGUUAAAAUAAGGCUAGUCCGUUAUCAACUUGAAAAAGUGG | |
| | CACCGAGUCGGUGCUsmUsmUsmU | |
| gRNA-#52 | mGsmGsmUsUUGCAGUCAGAUUGGCAGUUUUAGAGCUAGAAAUA | 605 |
| | GCAAGUUAAAAUAAGGCUAGUCCGUUAUCAACUUGAAAAAGUGG | |
| | CACCGAGUCGGUGCUsmUsmUsmU | |
| gRNA-#53 | mGsmUsmUsACUUAUUCUCUCUUUGUGUUUUAGAGCUAGAAAUA | 606 |
| | GCAAGUUAAAAUAAGGCUAGUCCGUUAUCAACUUGAAAAAGUGG | |
| | CACCGAGUCGGUGCUsmUsmUsmU | |
| gRNA-#54 | mGsmUsmUsCUGUCUUUUGGUCAGGACAACCGUCUAGCUAUAAG | 607 |
| | UGCUGCAGGGUGUGAGAAACUCCUAUUGCUGGACGAUGUCUCUU | |
| | ACGAGGCAUUAGCACCUUAUUCUCUCUUUGUUGAsmCsmUsmA | |
| gRNA-#55 | mGsmUsmUsCUGUCUUUUGGUCAGGACAACCGUCUAGCUAUAAG | 608 |
| | UGCUGCAGGGUGUGAGAAACUCCUAUUGCUGGACGAUGUCUCUU | |
| | ACGAGGCAUUAGCACAUAUUUGUAUGGGUUACUUsmAsmUsmU | |
| gRNA-#56 | mGsmUsmUsCUGUCUUUUGGUCAGGACAACCGUCUAGCUAUAAG | 609 |
| | UGCUGCAGGGUGUGAGAAACUCCUAUUGCUGGACGAUGUCUCUU | |
| | ACGAGGCAUUAGCACACUAAGUCAAUAAUCAGAAsmUsmCsmA | |
| gRNA-#57 | mGsmUsmUsUGCAGUCAGAUUGGCAGGUUUUAGAGCUAGAAAUA | 610 |
| | GCAAGUUAAAAUAAGGCUAGUCCGUUAUCAACUUGAAAAAGUGG | |
| | CACCGAGUCGGUGCUsmUsmUsmU | |
| gRNA-#58 | mGsmUsmUsCUGUCUUUUGGUCAGGACAACCGUCUAGCUAUAAG | 611 |
| | UGCUGCAGGGUGUGAGAAACUCCUAUUGCUGGACGAUGUCUCUU | |
| | ACGAGGCAUUAGCACGCAGUCAGAUUGGCAGGGAsmUsmAsmA | |
| gRNA-#59 | mUsmAsmCsAAAUAUGAACCUUGUCUGUUUUAGAGCUAGAAAUA | 612 |
| | GCAAGUUAAAAUAAGGCUAGUCCGUUAUCAACUUGAAAAAGUGG | |
| | CACCGAGUCGGUGCUsmUsmUsmU | |
| gRNA-#60 | mUsmAsmCsUCACUUCUCCUGAGCUAGUUUUAGAGCUAGAAAUA | 613 |
| | GCAAGUUAAAAUAAGGCUAGUCCGUUAUCAACUUGAAAAAGUGG | |
| | CACCGAGUCGGUGCUsmUsmUsmU | |
| gRNA-#61 | mUsmAsmUsAAAAGCCCCAGGCUGGGGUUUUAGAGCUAGAAAUA | 614 |
| | GCAAGUUAAAAUAAGGCUAGUCCGUUAUCAACUUGAAAAAGUGG | |
| | CACCGAGUCGGUGCUsmUsmUsmU | |
| gRNA-#62 | mUsmCsmAsCUUCUCCUGAGCUAGGCGUUUUAGAGCUAGAAAUA | 615 |
| | GCAAGUUAAAAUAAGGCUAGUCCGUUAUCAACUUGAAAAAGUGG | |
| | CACCGAGUCGGUGCUsmUsmUsmU | |
| gRNA-#63 | mUsmCsmAsGAUUGGCAGGGAUAAGCGUUUUAGAGCUAGAAAUA | 616 |
| | GCAAGUUAAAAUAAGGCUAGUCCGUUAUCAACUUGAAAAAGUGG | |
| | CACCGAGUCGGUGCUsmUsmUsmU | |
| gRNA-#64 | mUsmCsmAsGGAGAAGUGAGUAUAAAGUUUUAGAGCUAGAAAUA | 617 |
| | GCAAGUUAAAAUAAGGCUAGUCCGUUAUCAACUUGAAAAAGUGG | |
| | CACCGAGUCGGUGCUsmUsmUsmU | |
| gRNA-#65 | mUsmCsmUsGACUGCAAACCUGCUGAUGUUUUAGUACUCUGUAA | 618 |
| | UGAAAAUUACAGAAUCUACUAAAACAAGGCAAAAUGCCGUGUUU | |
| | AUCUCGUCAACUUGUUGGCGAGAUsmUsmUsmU | |
| gRNA-#66 | mUsmGsmAsGCUAGGCUGCUUAUCCCGUUUUAGAGCUAGAAAUA | 619 |
| | GCAAGUUAAAAUAAGGCUAGUCCGUUAUCAACUUGAAAAAGUGG | |
| | CACCGAGUCGGUGCUsmUsmUsmU | |
| gRNA-#67 | mUsmGsmCsCAAUCUGACUGCAAACCGUUUUAGAGCUAGAAAUA | 620 |
| | GCAAGUUAAAAUAAGGCUAGUCCGUUAUCAACUUGAAAAAGUGG | |
| | CACCGAGUCGGUGCUsmUsmUsmU | |
| gRNA-#68 | mUsmGsmCsUCUAAUCUCUCUAGACAGUUUUAGAGCUAGAAAUA | 621 |
| | GCAAGUUAAAAUAAGGCUAGUCCGUUAUCAACUUGAAAAAGUGG | |
| | CACCGAGUCGGUGCUsmUsmUsmU | |
| gRNA-#69 | mUsmGsmUsGAUGGCUGCUCCCAGCCGUUUUAGAGCUAGAAAUA | 622 |
| | GCAAGUUAAAAUAAGGCUAGUCCGUUAUCAACUUGAAAAAGUGG | |
| | CACCGAGUCGGUGCUsmUsmUsmU | |
| gRNA-#70 | mUsmUsmGsGCAGGGAUAAGCAGCCUGUUUUAGAGCUAGAAAUA | 623 |
| | GCAAGUUAAAAUAAGGCUAGUCCGUUAUCAACUUGAAAAAGUGG | |
| | CACCGAGUCGGUGCUsmUsmUsmU | |
| gRNA-#71 | mUsmUsmUsUAUACUCACUUCUCCUGGUUUUAGAGCUAGAAAUA | 624 |
| | GCAAGUUAAAAUAAGGCUAGUCCGUUAUCAACUUGAAAAAGUGG | |
| | CACCGAGUCGGUGCUsmUsmUsmU | |

Lowercase m indicates 2′-O-methylated nucleobases (e.g., mA, mC, mG, mU), and “s” indicates phosphorothioates.

TABLE 2A
Exemplary Spacer and Target Site Sequences.¹

| | | | | Target site | | |
|---|---|---|---|---|---|---|
| | | | | sequence (target | | |
| | | SEQ | Target | bases for base | SEQ | |
| | Spacer | ID | site | editing are in bold | ID | Target |
| sgRNA | sequence | NO | ID | and underlined) | NO | Base(s) |
| sgRNA_361 | UAUAGGAAAACCA | 475 | TSBTx2602 | TATAGGAAAACCAGTG | 469 | 4A |
| | GUGAGUC | | | AGTC | | |
| sgRNA_362 | UACUCACCUCUGC | 476 | TSBTx2603 | TACTCACCTCTGCATG | 470 | 6A, |
| | AUGCUCA | | | CTCA | | 7C |
| sgRNA_363 | ACUCACCUCUGCA | 625 | TSBTx2604 | ACTCACCTCTGCATGC | 639 | 5A, |
| | UGCUCAU | | | TCAT | | 6C |
| sgRNA_364 | UACCACCUAUGAG | 626 | TSBTx2605 | TACCACCTATGAGAGA | 640 | 7C |
| | AGAAGAC | | | AGAC | | |
| sgRNA_365 | AUACUCACCUCUG | 627 | TSBTx2606 | ATACTCACCTCTGCAT | 641 | 7A, |
| | CAUGCUCA | | | GCTCA | | 8C |
| sgRNA_366 | ACUGGUUUUCCUA | 628 | TSBTx2607 | ACTGGTTTTCCTATAA | 642 | 11C |
| | UAAGGUGU | | | GGTGT | | |
| sgRNA_367 | UUGGCAGGAUGGC | 629 | TSBTx2608 | TTGGCAGGATGGCTTC | 643 | 6A, |
| | UUCUCAUCG | | | TCATCG | | 9A |
| sgRNA_368 | UCCUAUAAGGUGU | 630 | TSBTx2609 | GTTTTCCTATAAGGTG | 644 | |
| | GAAAGUCUG | | | TGAAAGTCTG | | |
| sgRNA_369 | UGAGCCCAUGCAG | 631 | TSBTx2610 | GTTGTGAGCCCATGCA | 645 | |
| | CUCUCCAGA | | | GCTCTCCAGA | | |
| sgRNA_370 | CUCCUCAGUUGUG | 632 | TSBTx2611 | ATTCCTCCTCAGTTGT | 646 | |
| | AGCCCAUGC | | | GAGCCCATGC | | |
| sgRNA_371 | GUAGAAGGGAUAU | 633 | TSBTx2612 | ATTTGTAGAAGGGATA | 647 | |
| | ACAAAGUGG | | | TACAAAGTGG | | |
| sgRNA_372 | CCACUUUGUAUAU | 634 | TSBTx2613 | ATTTCCACTTTGTATA | 648 | |
| | CCCUUCUAC | | | TCCCTTCTAC | | |
| sgRNA_373 | GGUGUCUAUUUCC | 635 | TSBTx2614 | ATTTGGTGTCTATTTC | 649 | |
| | ACUUUGUAU | | | CACTTTGTAT | | |
| sgRNA_374 | CAUGAGCAUGCAG | 636 | TSBTx2615 | ATTCCATGAGCATGCA | 650 | |
| | AGGUGAGUA | | | GAGGTGAGTA | | |
| sgRNA_375 | GGCUAUCGUCACC | 637 | | GGCTATCGTCACCAAT | 651 | 5A |
| | AAUCCCA | | | CCCA | | |
| sgRNA_376 | GCUAUCGUCACCA | 638 | | GCTATCGTCACCAATC | 652 | 4A |
| | AUCCCAA | | | CCAA | | |
| sgRNA_377 | GGCUAUCGUCACC | 637 | | GGCTATCGTCACCAAT | 651 | 5A |
| | AAUCCCA | | | CCCA | | |

1One of skill in the art will understand that some of the target site sequences correspond to a reverse-complement to the above-provided transthyretin polynucleotide sequence; i.e., the target sequences may correspond to either strand of a dsDNA molecule encoding a transthyretin polynucleotide. Further, it is to be understood that a C base can be targeted by a cytidine deaminase and that an A base can be targeted by an adenine deaminase.

TABLE 2B
Exemplary Spacer and Target Site Sequences.

| | | SEQ | Target Site/ | SEQ |
|---|---|---|---|---|
| gRNA_ | Spacer | ID | Protospacer | ID |
| Name | Sequence | NO: | Sequence | NO: |
| gRNA1747 | AAGAGAGAAUAAGU | 472 | AAGAGAGAATAAGTAA | 785 |
| | AACCCAU | | CCCAT | |
| gRNA1748 | AAGCAGCCUAGCUC | 654 | AAGCAGCCTAGCTCAG | 786 |
| | AGGAGAA | | GAGAA | |
| gRNA1749 | AAGUCCACUCAUUC | 655 | AAGTCCACTCATTCTT | 787 |
| | UUGGCA | | GGCA | |
| gRNA1750 | ACGAUGAGAAGCCA | 656 | ACGATGAGAAGCCATC | 788 |
| | UCCUGCC | | CTGCC | |
| gRNA1751 | AGACAAGGUUCAUA | 657 | AGACAAGGTTCATATT | 789 |
| | UUUGUA | | TGTA | |
| gRNA1752 | AGGCUGGGAGCAGC | 658 | AGGCTGGGAGCAGCCA | 790 |
| | CAUCAC | | TCAC | |
| gRNA1753 | AUAAGUAACCCAUA | 659 | ATAAGTAACCCATACA | 791 |
| | CAAAUA | | AATA | |
| gRNA1754 | AUACUCACUUCUCC | 660 | ATACTCACTTCTCCTG | 792 |
| | UGAGCU | | AGCT | |
| gRNA1755 | AUUAUUGACUUAGU | 661 | ATTATTGACTTAGTCA | 793 |
| | CAACAA | | ACAA | |
| gRNA1756 | CAAAUAUGAACCUU | 662 | CAAATATGAACCTTGT | 794 |
| | GUCUAG | | CTAG | |
| gRNA1757 | CAGAAGUCCACUCA | 663 | CAGAAGTCCACTCATT | 795 |
| | UUCUUGG | | CTTGG | |
| gRNA1758 | CAGGCUGGGAGCAG | 664 | CAGGCTGGGAGCAGCC | 796 |
| | CCAUCAC | | ATCAC | |
| gRNA1759 | CCAUCCUGCCAAGA | 665 | CCATCCTGCCAAGAAT | 797 |
| | AUGAGU | | GAGT | |
| gRNA1760 | CCUGCUGAUUCUGA | 666 | CCTGCTGATTCTGATT | 798 |
| | UUAUUGA | | ATTGA | |
| gRNA1761 | CGAUGCUCUAAUCU | 667 | CGATGCTCTAATCTCT | 799 |
| | CUCUAGA | | CTAGA | |
| gRNA1762 | CUAAGUCAAUAAUC | 668 | CTAAGTCAATAATCAG | 800 |
| | AGAAUCA | | AATCA | |
| gRNA1763 | CUAGACAAGGUUCA | 669 | CTAGACAAGGTTCATA | 801 |
| | UAUUUGU | | TTTGT | |
| gRNA1764 | GAACCUUGUCUAGA | 670 | GAACCTTGTCTAGAGA | 802 |
| | GAGAUU | | GATT | |
| gRNA1765 | GAAGUCCACUCAUU | 671 | GAAGTCCACTCATTCT | 803 |
| | CUUGGC | | TGGC | |
| gRNA1766 | GAAUCAGCAGGUUU | 672 | GAATCAGCAGGTTTGC | 804 |
| | GCAGUC | | AGTC | |
| gRNA1767 | GAAUGAGUGGACUU | 673 | GAATGAGTGGACTTCT | 805 |
| | CUGUGA | | GTGA | |
| gRNA1768 | GACUGCAAACCUGC | 674 | GACTGCAAACCTGCTG | 806 |
| | UGAUUC | | ATTC | |
| gRNA1769 | GACUUAGUCAACAA | 675 | GACTTAGTCAACAAAG | 807 |
| | AGAGAGA | | AGAGA | |
| gRNA1770 | GAUAAGCAGCCUAG | 676 | GATAAGCAGCCTAGCT | 808 |
| | CUCAGG | | CAGG | |
| gRNA1771 | GAUGAGAAGCCAUC | 677 | GATGAGAAGCCATCCT | 809 |
| | CUGCCA | | GCCA | |
| gRNA1772 | GCCAUCCUGCCAAG | 472 | GCCATCCTGCCAAGAA | 467 |
| | AAUGAG | | TGAG | |
| gRNA1773 | GCUUUUAUACUCAC | 654 | GCTTTTATACTCACTT | 811 |
| | UUCUCC | | CTCC | |
| gRNA1774 | GGAUAAGCAGCCUA | 655 | GGATAAGCAGCCTAGC | 812 |
| | GCUCAGG | | TCAGG | |
| gRNA1775 | GUCUAGAGAGAUUA | 656 | GTCTAGAGAGATTAGA | 813 |
| | GAGCAU | | GCAT | |
| gRNA1776 | GUGAUGGCUGCUCC | 657 | GTGATGGCTGCTCCCA | 814 |
| | CAGCCU | | GCCT | |
| gRNA1777 | UACUUAUUCUCUCU | 658 | TACTTATTCTCTCTTT | 815 |
| | UUGUUGA | | GTTGA | |
| gRNA1778 | UAUUCUCUCUUUGU | 659 | TATTCTCTCTTTGTTG | 816 |
| | UGACUAA | | ACTAA | |
| gRNA1779 | UAUUGACUUAGUCA | 660 | TATTGACTTAGTCAAC | 817 |
| | ACAAAG | | AAAG | |
| gRNA1780 | UAUUGACUUAGUCA | 661 | TATTGACTTAGTCAAC | 818 |
| | ACAAAGA | | AAAGA | |
| gRNA1781 | UCAGAAUCAGCAGG | 662 | TCAGAATCAGCAGGTT | 819 |
| | UUUGCAG | | TGCAG | |
| gRNA1782 | UCCACUCAUUCUUG | 663 | TCCACTCATTCTTGGC | 820 |
| | GCAGGA | | AGGA | |
| gRNA1783 | UCUCUCUUUGUUGA | 664 | TCTCTCTTTGTTGACT | 821 |
| | CUAAGUC | | AAGTC | |
| gRNA1784 | UGAGAAGCCAUCCU | 665 | TGAGAAGCCATCCTGC | 822 |
| | GCCAAGA | | CAAGA | |
| gRNA1785 | UGAGCUAGGCUGCU | 666 | TGAGCTAGGCTGCTTA | 823 |
| | UAUCCCU | | TCCCT | |
| gRNA1786 | UGAGUAUAAAAGCC | 667 | TGAGTATAAAAGCCCC | 824 |
| | CCAGGC | | AGGC | |
| gRNA1787 | UGAUGGCUGCUCCC | 668 | TGATGGCTGCTCCCAG | 825 |
| | AGCCUG | | CCTG | |
| gRNA1788 | UGCCAAGAAUGAGU | 669 | TGCCAAGAATGAGTGG | 826 |
| | GGACUUC | | ACTTC | |
| gRNA1789 | UGCCAAUCUGACUG | 670 | TGCCAATCTGACTGCA | 827 |
| | CAAACCU | | AACCT | |
| gRNA1790 | UGUUGACUAAGUCA | 671 | TGTTGACTAAGTCAAT | 828 |
| | AUAAUC | | AATC | |
| gRNA1791 | UUGACUUAGUCAAC | 672 | TTGACTTAGTCAACAA | 829 |
| | AAAGAG | | AGAG | |
| gRNA1792 | UUUGUUGACUAAGU | 673 | TTTGTTGACTAAGTCA | 830 |
| | CAAUAAU | | ATAAT | |
| gRNA1746 | AACCUGCUGAUUCU | 674 | AACCTGCTGATTCTGA | 831 |
| | GAUUAU | | TTAT | |
| gRNA1594 | CAACUUACCCAGAG | 675 | CAACTTACCCAGAGGC | 832 |
| | GCAAAU | | AAAT | |
| gRNA1595 | AAUGGCUCCCAGGU | 676 | AATGGCTCCCAGGTGT | 833 |
| | GUCAUC | | CATC | |
| gRNA1596 | GGCUCCCAGGUGUC | 677 | GGCTCCCAGGTGTCAT | 834 |
| | AUCAGC | | CAGC | |
| gRNA1597 | CUCUCAUAGGUGGU | 653 | CTCTCATAGGTGGTAT | 835 |
| | AUUCAC | | TCAC | |
| gRNA1598 | UAUAGGAAAACCAG | 475 | TATAGGAAAACCAGTG | 469 |
| | UGAGUC | | AGTC | |
| gRNA1599 | UACUCACCUCUGCA | 476 | TACTCACCTCTGCATG | 470 |
| | UGCUCA | | CTCA | |
| gRNA1600 | GCAACUUACCCAGA | 474 | GCAACTTACCCAGAGG | 468 |
| | GGCAAA | | CAAA | |
| gRNA1601 | UCUGUAUACUCACC | 707 | TCTGTATACTCACCTC | 836 |
| | UCUGCA | | TGCA | |
| gRNA1602 | GAAACACUCACCGU | 708 | GAAACACTCACCGTAG | 837 |
| | AGGGCC | | GGCC | |
| gRNA1603 | CUCUACACCCAGGG | 709 | CTCTACACCCAGGGCA | 838 |
| | CACCGG | | CCGG | |
| gRNA1604 | ACACCUUAUAGGAA | 710 | ACACCTTATAGGAAAA | 839 |
| | AACCAG | | CCAG | |
| gRNA1605 | AUAGGAAAACCAGU | 711 | ATAGGAAAACCAGTGA | 840 |
| | GAGUCU | | GTCT | |
| gRNA1606 | ACUCACCUCUGCAU | 625 | ACTCACCTCTGCATGC | 639 |
| | GCUCAU | | TCAT | |
| gRNA1607 | CUCACCGUAGGGCC | 713 | CTCACCGTAGGGCCAG | 841 |
| | AGCCUC | | CCTC | |
| gRNA-#1 | AAAAGCCCCAGGCU | 714 | AAAAGCCCCAGGCTGG | 842 |
| | GGGAGC | | GAGC | |
| gRNA-#2 | AAGUGAGUAUAAAA | 715 | AAGTGAGTATAAAAGC | 843 |
| | GCCCCA | | CCCA | |
| gRNA-#3 | AAUAAUCAGAAUCA | 716 | AATAATCAGAATCAGC | 844 |
| | GCAGGUU | | AGGTT | |
| gRNA-#4 | AAUAUGAACCUUGU | 717 | AATATGAACCTTGTCT | 845 |
| | CUAGAG | | AGAG | |
| gRNA-#5 | AAUGAGUGGACUUC | 718 | AATGAGTGGACTTCTG | 846 |
| | UGUGAU | | TGAT | |
| gRNA-#6 | ACAAAUAUGAACCU | 719 | ACAAATATGAACCTTG | 847 |
| | UGUCUAG | | TCTAG | |
| gRNA-#7 | ACAGAAGUCCACUC | 720 | ACAGAAGTCCACTCAT | 848 |
| | AUUCUU | | TCTT | |
| gRNA-#8 | ACCUUGUCUAGAGA | 721 | ACCTTGTCTAGAGAGA | 849 |
| | GAUUAG | | TTAG | |
| gRNA-#9 | AGAAGCCAUCCUGC | 722 | AGAAGCCATCCTGCCA | 850 |
| | CAAGAA | | AGAA | |
| gRNA-#10 | AGCAGGUUUGCAGU | 723 | AGCAGGTTTGCAGTCA | 851 |
| | CAGAUU | | GATT | |
| gRNA-#11 | AGGGAUAAGCAGCC | 724 | AGGGATAAGCAGCCTA | 852 |
| | UAGCUC | | GCTC | |
| gRNA-#12 | AGGUUUGCAGUCAG | 725 | AGGTTTGCAGTCAGAT | 853 |
| | AUUGGC | | TGGC | |
| gRNA-#13 | AGUAUAAAAGCCCC | 726 | AGTATAAAAGCCCCAG | 854 |
| | AGGCUG | | GCTG | |
| gRNA-#14 | AGUCAAUAAUCAGA | 727 | AGTCAATAATCAGAAT | 855 |
| | AUCAGC | | CAGC | |
| gRNA-#15 | AUAAUCAGAAUCAG | 728 | ATAATCAGAATCAGCA | 856 |
| | CAGGUU | | GGTT | |
| gRNA-#16 | UUGACUUAGUCAAC | 729 | TTGACTTAGTCAACAA | 857 |
| | AAAGAGAG | | AGAGAG | |
| gRNA-#17 | UCUCUUUGUUGACU | 730 | TCTCTTTGTTGACTAA | 858 |
| | AAGUCAAU | | GTCAAT | |
| gRNA-#18 | UGAUUAUUGACUUA | 731 | TGATTATTGACTTAGT | 859 |
| | GUCAACAA | | CAACAA | |
| gRNA-#19 | UUGGCAGGAUGGCU | 629 | TTGGCAGGATGGCTTC | 643 |
| (sgRNA_ | UCUCAUCG | | TCATCG | |
| 367) | | | | |
| gRNA-#20 | ACUUAGUCAACAAA | 733 | ACTTAGTCAACAAAGA | 860 |
| | GAGAGAAU | | GAGAAT | |
| gRNA-#21 | GCAGGGAUAAGCAG | 734 | GCAGGGATAAGCAGCC | 861 |
| | CCUAGCUC | | TAGCTC | |
| gRNA-#22 | GUAUGGGUUACUUA | 735 | GTATGGGTTACTTATT | 862 |
| | UUCUCUCU | | CTCTCT | |
| gRNA-#23 | CAAGAAUGAGUGGA | 736 | CAAGAATGAGTGGACT | 863 |
| | CUUCUG | | TCTG | |
| gRNA-#24 | CAAUCUGACUGCAA | 737 | CAATCTGACTGCAAAC | 864 |
| | ACCUGC | | CTGC | |
| gRNA-#25 | CACAGAAGUCCACU | 738 | CACAGAAGTCCACTCA | 865 |
| | CAUUCU | | TTCT | |
| gRNA-#26 | CAGACGAUGAGAAG | 739 | CAGACGATGAGAAGCC | 866 |
| | CCAUCC | | ATCC | |
| gRNA-#27 | CAGCAGGUUUGCAG | 740 | CAGCAGGTTTGCAGTC | 867 |
| | UCAGAU | | AGAT | |
| gRNA-#28 | CAGGAUGGCUUCUC | 741 | CAGGATGGCTTCTCAT | 868 |
| | AUCGUC | | CGTC | |
| gRNA-#29 | CAGGUUUGCAGUCA | 742 | CAGGTTTGCAGTCAGA | 869 |
| | GAUUGGC | | TTGGC | |
| gRNA-#30 | CAGUCAGAUUGGCA | 743 | CAGTCAGATTGGCAGG | 870 |
| | GGGAUA | | GATA | |
| gRNA-#31 | CCACUCAUUCUUGG | 744 | CCACTCATTCTTGGCA | 871 |
| | CAGGAU | | GGAT | |
| gRNA-#32 | CUAAGUCAAUAAUC | 745 | CTAAGTCAATAATCAG | 872 |
| | AGAAUC | | AATC | |
| gRNA-#33 | GUCAACAAAGAGAG | 746 | GTCAACAAAGAGAGAA | 873 |
| | AAUAAGUA | | TAAGTA | |
| gRNA-#34 | CUUAUCCCUGCCAA | 747 | CTTATCCCTGCCAATC | 874 |
| | UCUGAC | | TGAC | |
| gRNA-#35 | UCCCUGCCAAUCUG | 748 | TCCCTGCCAATCTGAC | 875 |
| | ACUGCAAA | | TGCAAA | |
| gRNA-#36 | UUCUCUCUUUGUUG | 749 | TTCTCTCTTTGTTGAC | 876 |
| | ACUAAGUC | | TAAGTC | |
| gRNA-#37 | UCCUGAGCUAGGCU | 750 | TCCTGAGCTAGGCTGC | 877 |
| | GCUUAUCC | | TTATCC | |
| gRNA-#38 | CUUCUGUGAUGGCU | 751 | CTTCTGTGATGGCTGC | 878 |
| | GCUCCC | | TCCC | |
| gRNA-#39 | UGUGAUGGCUGCUC | 752 | TGTGATGGCTGCTCCC | 879 |
| | CCAGCCUG | | AGCCTG | |
| gRNA-#40 | GCAGGAUGGCUUCU | 753 | GCAGGATGGCTTCTCA | 880 |
| | CAUCGUCU | | TCGTCT | |
| gRNA-#41 | UCUAGAGAGAUUAG | 754 | TCTAGAGAGATTAGAG | 881 |
| | AGCAUCGG | | CATCGG | |
| gRNA-#42 | GUUGACUAAGUCAA | 755 | GTTGACTAAGTCAATA | 882 |
| | UAAUCAGA | | ATCAGA | |
| gRNA-#43 | UAUACUCACUUCUC | 756 | TATACTCACTTCTCCT | 883 |
| | CUGAGCUA | | GAGCTA | |
| gRNA-#44 | GAAGUGAGUAUAAA | 757 | GAAGTGAGTATAAAAG | 884 |
| | AGCCCC | | CCCC | |
| gRNA-#45 | GACAAGGUUCAUAU | 758 | GACAAGGTTCATATTT | 885 |
| | UUGUAU | | GTAT | |
| gRNA-#46 | GAGUAUAAAAGCCC | 759 | GAGTATAAAAGCCCCA | 886 |
| | CAGGCU | | GGCT | |
| gRNA-#47 | GAGUGGACUUCUGU | 760 | GAGTGGACTTCTGTGA | 887 |
| | GAUGGC | | TGGC | |
| gRNA-#48 | GAUGGCUGCUCCCA | 761 | GATGGCTGCTCCCAGC | 888 |
| | GCCUGG | | CTGG | |
| gRNA-#49 | GCAGCCUAGCUCAG | 762 | GCAGCCTAGCTCAGGA | 889 |
| | GAGAAG | | GAAG | |
| gRNA-#50 | GCUGCUUAUCCCUG | 763 | GCTGCTTATCCCTGCC | 890 |
| | CCAAUC | | AATC | |
| gRNA-#51 | GGGAUAAGCAGCCU | 764 | GGGATAAGCAGCCTAG | 891 |
| | AGCUCA | | CTCA | |
| gRNA-#52 | GGUUUGCAGUCAGA | 765 | GGTTTGCAGTCAGATT | 892 |
| | UUGGCA | | GGCA | |
| gRNA-#53 | GUUACUUAUUCUCU | 766 | GTTACTTATTCTCTCT | 893 |
| | CUUUGU | | TTGT | |
| gRNA-#54 | CUUAUUCUCUCUUU | 767 | CTTATTCTCTCTTTGT | 894 |
| | GUUGACUA | | TGACTA | |
| gRNA-#55 | AUAUUUGUAUGGGU | 768 | ATATTTGTATGGGTTA | 895 |
| | UACUUAUU | | CTTATT | |
| gRNA-#56 | ACUAAGUCAAUAAU | 769 | ACTAAGTCAATAATCA | 896 |
| | CAGAAUCA | | GAATCA | |
| gRNA-#57 | GUUUGCAGUCAGAU | 770 | GTTTGCAGTCAGATTG | 897 |
| | UGGCAG | | GCAG | |
| gRNA-#58 | GCAGUCAGAUUGGC | 771 | GCAGTCAGATTGGCAG | 898 |
| | AGGGAUAA | | GGATAA | |
| gRNA-#59 | UACAAAUAUGAACC | 772 | TACAAATATGAACCTT | 899 |
| | UUGUCU | | GTCT | |
| gRNA-#60 | UACUCACUUCUCCU | 773 | TACTCACTTCTCCTGA | 900 |
| | GAGCUA | | GCTA | |
| gRNA-#61 | UAUAAAAGCCCCAG | 774 | TATAAAAGCCCCAGGC | 901 |
| | GCUGGG | | TGGG | |
| gRNA-#62 | UCACUUCUCCUGAG | 775 | TCACTTCTCCTGAGCT | 902 |
| | CUAGGC | | AGGC | |
| gRNA-#63 | UCAGAUUGGCAGGG | 776 | TCAGATTGGCAGGGAT | 903 |
| | AUAAGC | | AAGC | |
| gRNA-#64 | UCAGGAGAAGUGAG | 777 | TCAGGAGAAGTGAGTA | 904 |
| | UAUAAA | | TAAA | |
| gRNA-#65 | UCUGACUGCAAACC | 778 | TCTGACTGCAAACCTG | 905 |
| | UGCUGAU | | CTGAT | |
| gRNA-#66 | UGAGCUAGGCUGCU | 779 | TGAGCTAGGCTGCTTA | 906 |
| | UAUCCC | | TCCC | |
| gRNA-#67 | UGCCAAUCUGACUG | 780 | TGCCAATCTGACTGCA | 907 |
| | CAAACC | | AACC | |
| gRNA-#68 | UGCUCUAAUCUCUC | 781 | TGCTCTAATCTCTCTA | 908 |
| | UAGACA | | GACA | |
| gRNA-#69 | UGUGAUGGCUGCUC | 782 | TGTGATGGCTGCTCCC | 909 |
| | CCAGCC | | AGCC | |
| gRNA-#70 | UUGGCAGGGAUAAG | 783 | TTGGCAGGGATAAGCA | 910 |
| | CAGCCU | | GCCT | |
| gRNA-#71 | UUUUAUACUCACUU | 784 | TTTTATACTCACTTCT | 911 |
| | CUCCUG | | CCTG | |

TABLE 2C
Exemplary human TTR target site sequences and base editor +
guide RNA combinations.

| | | | | | Target Site/ | |
|---|---|---|---|---|---|---|
| | | | | | Protospacer + | SEQ |
| gRNA | | Cas | Editor | Editing | PAM | ID |
| Name | Editor Name | Name | Alias | Strategy | Sequence | NO |
| gRNA1594 | CBE_NGC_20nt_ | spCas9 | spCas9 | Splice | CAACTTACCCAG | 912 |
| | t_4-9_009 | | NGC | Site | AGGCAAATGGC | |
| | | | CBE | | | |
| gRNA1594 | ABE_NGC_20nt_ | spCas9 | spCas9 | Splice | CAACTTACCCAG | 912 |
| | 3-9_008 | | NGC | Site | AGGCAAATGGC | |
| | | | ABE | | | |
| gRNA1594 | ABE_NGC_20nt_ | spCas9 | spCas9 | Splice | CAACTTACCCAG | 912 |
| | 3-12_020 | | NGC IBE | Site | AGGCAAATGGC | |
| gRNA1595 | CBE_NGC_20nt_ | spCas9 | spCas9 | Stop | AATGGCTCCCAG | 913 |
| | 4-9_009 | | NGC | Codon | GTGTCATCAGC | |
| | | | CBE | | | |
| gRNA1596 | CBE_NGC_20nt_ | spCas9 | spCas9 | Stop | GGCTCCCAGGTG | 914 |
| | 4-9_009 | | NGC | Codon | TCATCAGCAGC | |
| | | | CBE | | | |
| gRNA1597 | ABE_NGC_20nt_ | spCas9 | spCas9 | Splice | CTCTCATAGGTG | 915 |
| | 3-9_008 | | NGC | Site | GTATTCACAGC | |
| | | | ABE | | | |
| gRNA1597 | ABE_NGC_20nt_ | spCas9 | spCas9 | Splice | CTCTCATAGGTG | 915 |
| | 3-12_020 | | NGC IBE | Site | GTATTCACAGC | |
| gRNA1598 | ABE_NGG_20nt_ | spCas9 | spCas9 | Splice | TATAGGAAAACC | 916 |
| | 3-12_018 | | IBE | Site | AGTGAGTCTGG | |
| gRNA1599 | ABE_NGG_20nt_ | spCas9 | spCas9 | Splice | TACTCACCTCTG | 917 |
| | 3-12_018 | | IBE | Site | CATGCTCATGG | |
| gRNA1600 | ABE_NGG_20nt_ | spCas9 | spCas9 | Splice | GCAACTTACCCA | 918 |
| | 3-12_018 | | IBE | Site | GAGGCAAATGG | |
| gRNA1601 | ABE_NGC_20nt_ | spCas9 | spCas9 | Splice | TCTGTATACTCA | 919 |
| | 3-12_020 | | NGC IBE | Site | CCTCTGCATGC | |
| gRNA1602 | ABE_NGC_20nt_ | spCas9 | spCas9 | Splice | GAAACACTCACC | 920 |
| | 3-12_020 | | NGC IBE | Site | GTAGGGCCAGC | |
| gRNA1603 | ABE_NGA_20nt_ | spCas9 | spCas9 | Splice | CTCTACACCCAG | 921 |
| | 3-12_019 | | VRQR | Site | GGCACCGGTGA | |
| | | | IBE | | | |
| gRNA1604 | ABE_NGA_20nt_ | spCas9 | spCas9 | Splice | ACACCTTATAGG | 922 |
| | 3-12_019 | | VRQR | Site | AAAACCAGTGA | |
| | | | IBE | | | |
| gRNA1605 | ABE_NGA_20nt_ | spCas9 | spCas9 | Splice | ATAGGAAAACCA | 923 |
| | 3-12_019 | | VRQR | Site | GTGAGTCTGGA | |
| | | | IBE | | | |
| gRNA1606 | ABE_NGA_20nt_ | spCas9 | spCas9 | Splice | ACTCACCTCTGC | 924 |
| | 3-12_019 | | VRQR | Site | ATGCTCATGGA | |
| | | | IBE | | | |
| gRNA1607 | ABE_NGA_20nt_ | spCas9 | spCas9 | Splice | CTCACCGTAGGG | 925 |
| | 3-12_019 | | VRQR | Site | CCAGCCTCAGA | |
| | | | IBE | | | |
| gRNA1746 | ABE_NGA_20nt_ | spCas9 | spCas9 | TTR | AACCTGCTGATT | 926 |
| | 3-9_005 | | VRQR | Promoter | CTGATTATTGA | |
| | | | ABE | | | |
| gRNA1746 | CBE_NGA_20nt_ | spCas9 | spCas9 | TTR | AACCTGCTGATT | 926 |
| | 4-9_006 | | VRQR | Promoter | CTGATTATTGA | |
| | | | CBE | | | |
| gRNA1746 | ABE_NGA_20nt_ | spCas9 | spCas9 | TTR | AACCTGCTGATT | 926 |
| | 3-12_019 | | VRQR | Promoter | CTGATTATTGA | |
| | | | IBE | | | |
| gRNA1747 | ABE_NNNRRT_ | saCas9 | saCas9 | TTR | AAGAGAGAATAA | 927 |
| | 21nt_5-14_014 | | KKH | Promoter | GTAACCCATACA | |
| | | | ABE | | AAT | |
| gRNA1747 | CBE_NNNRRT_ | saCas9 | saCas9 | TTR | AAGAGAGAATAA | 927 |
| | 21nt_3-12_015 | | KKH | Promoter | GTAACCCATACA | |
| | | | CBE | | AAT | |
| gRNA1748 | ABE_NNGRRT_ | saCas9 | saCas9 | TTR | AAGCAGCCTAGC | 928 |
| | 21nt_5-14_011 | | ABE | Promoter | TCAGGAGAAGTG | |
| | | | | | AGT | |
| gRNA1748 | CBE_NNGRRT_ | saCas9 | saCas9 | TTR | AAGCAGCCTAGC | 928 |
| | 21nt_3-12_012 | | CBE | Promoter | TCAGGAGAAGTG | |
| | | | | | AGT | |
| gRNA1749 | ABE_NGA_20nt_ | spCas9 | spCas9 | TTR | AAGTCCACTCAT | 929 |
| | 3-9_005 | | VRQR | Promoter | TCTTGGCAGGA | |
| | | | ABE | | | |
| gRNA1749 | CBE_NGA_20nt_ | spCas9 | spCas9 | TTR | AAGTCCACTCAT | 929 |
| | 4-9_006 | | VRQR | Promoter | TCTTGGCAGGA | |
| | | | CBE | | | |
| gRNA1749 | ABE_NGA_20nt_ | spCas9 | spCas9 | TTR | AAGTCCACTCAT | 929 |
| | 3-12_019 | | VRQR | Promoter | TCTTGGCAGGA | |
| | | | IBE | | | |
| gRNA1750 | ABE_NNGRRT_ | saCas9 | saCas9 | TTR | ACGATGAGAAGC | 930 |
| | 21nt_5-14_011 | | ABE | Promoter | CATCCTGCCAAG | |
| | | | | | AAT | |
| gRNA1750 | CBE_NNGRRT | saCas9 | saCas9 | TTR | ACGATGAGAAGC | 930 |
| | 21nt_3-12_012 | | CBE | Promoter | CATCCTGCCAAG | |
| | | | | | AAT | |
| gRNA1751 | ABE_NGG_20nt_ | spCas9 | spCas9 | TTR | AGACAAGGTTCA | 931 |
| | 3-9_002 | | ABE | Promoter | TATTTGTATGG | |
| gRNA1751 | CBE_NGG_20nt_ | spCas9 | spCas9 | TTR | AGACAAGGTTCA | 931 |
| | 4-9_003 | | CBE | Promoter | TATTTGTATGG | |
| gRNA1751 | ABE_NGG_20nt_ | spCas9 | spCas9 | TTR | AGACAAGGTTCA | 931 |
| | 3-12_018 | | IBE | Promoter | TATTTGTATGG | |
| gRNA1752 | ABE_NGA_20nt_ | spCas9 | spCas9 | TTR | AGGCTGGGAGCA | 932 |
| | 3-9_005 | | VRQR | Promoter | GCCATCACAGA | |
| | | | ABE | | | |
| gRNA1752 | CBE_NGA_20nt_ | spCas9 | spCas9 | TTR | AGGCTGGGAGCA | 932 |
| | 4-9_006 | | VRQR | Promoter | GCCATCACAGA | |
| | | | CBE | | | |
| gRNA1752 | ABE_NGA_20nt_ | spCas9 | spCas9 | TTR | AGGCTGGGAGCA | 932 |
| | 3-12_019 | | VRQR | Promoter | GCCATCACAGA | |
| | | | IBE | | | |
| gRNA1753 | ABE_NGA_20nt_ | spCas9 | spCas9 | TTR | ATAAGTAACCCA | 933 |
| | 3-9_005 | | VRQR | Promoter | TACAAATATGA | |
| | | | ABE | | | |
| gRNA1753 | CBE_NGA_20nt_ | spCas9 | spCas9 | TTR | ATAAGTAACCCA | 933 |
| | 4-9_006 | | VRQR | Promoter | TACAAATATGA | |
| | | | CBE | | | |
| gRNA1753 | ABE_NGA_20nt_ | spCas9 | spCas9 | TTR | ATAAGTAACCCA | 933 |
| | 3-12_019 | | VRQR | Promoter | TACAAATATGA | |
| | | | IBE | | | |
| gRNA1754 | ABE_NGG_20nt_ | spCas9 | spCas9 | TTR | ATACTCACTTCT | 934 |
| | 3-9_002 | | ABE | Promoter | CCTGAGCTAGG | |
| gRNA1754 | CBE_NGG_20nt_ | spCas9 | spCas9 | TTR | ATACTCACTTCT | 934 |
| | 4-9_003 | | CBE | Promoter | CCTGAGCTAGG | |
| gRNA1754 | ABE_NGG_20nt_ | spCas9 | spCas9 | TTR | ATACTCACTTCT | 934 |
| | 3-12_018 | | IBE | Promoter | CCTGAGCTAGG | |
| gRNA1755 | ABE_NGA_20nt_ | spCas9 | spCas9 | TTR | ATTATTGACTTA | 935 |
| | 3-9_005 | | VRQR | Promoter | GTCAACAAAGA | |
| | | | ABE | | | |
| gRNA1755 | CBE_NGA_20nt_ | spCas9 | spCas9 | TTR | ATTATTGACTTA | 935 |
| | 4-9_006 | | VRQR | Promoter | GTCAACAAAGA | |
| | | | CBE | | | |
| gRNA1755 | ABE_NGA_20nt_ | spCas9 | spCas9 | TTR | ATTATTGACTTA | 935 |
| | 3-12_019 | | VRQR | Promoter | GTCAACAAAGA | |
| | | | IBE | | | |
| gRNA1756 | ABE_NGA_20nt_ | spCas9 | spCas9 | TTR | CAAATATGAACC | 936 |
| | 3-9_005 | | VRQR | Promoter | TTGTCTAGAGA | |
| | | | ABE | | | |
| gRNA1756 | CBE_NGA_20nt_ | spCas9 | spCas9 | TTR | CAAATATGAACC | 936 |
| | 4-9_006 | | VRQR | Promoter | TTGTCTAGAGA | |
| | | | CBE | | | |
| gRNA1756 | ABE_NGA_20nt_ | spCas9 | spCas9 | TTR | CAAATATGAACC | 936 |
| | 3-12_019 | | VRQR | Promoter | TTGTCTAGAGA | |
| | | | IBE | | | |
| gRNA1757 | ABE_NNGRRT_ | saCas9 | saCas9 | TTR | CAGAAGTCCACT | 937 |
| | 21nt_5-14_011 | | ABE | Promoter | CATTCTTGGCAG | |
| | | | | | GAT | |
| gRNA1757 | CBE_NNGRRT_ | saCas9 | saCas9 | TTR | CAGAAGTCCACT | 937 |
| | 21nt_3-12_012 | | CBE | Promoter | CATTCTTGGCAG | |
| | | | | | GAT | |
| gRNA1758 | ABE_NNNRRT_ | saCas9 | saCas9 | TTR | CAGGCTGGGAGC | 938 |
| | 21nt_5-14_014 | | KKH | Promoter | AGCCATCACAGA | |
| | | | ABE | | AGT | |
| gRNA1758 | CBE_NNNRRT_ | saCas9 | saCas9 | TTR | CAGGCTGGGAGC | 938 |
| | 21nt_3-12_015 | | KKH | Promoter | AGCCATCACAGA | |
| | | | CBE | | AGT | |
| gRNA1759 | ABE_NGA_20nt_ | spCas9 | spCas9 | TTR | CCATCCTGCCAA | 939 |
| | 3-9_005 | | VRQR | Promoter | GAATGAGTGGA | |
| | | | ABE | | | |
| gRNA1759 | CBE_NGA_20nt_ | spCas9 | spCas9 | TTR | CCATCCTGCCAA | 939 |
| | 4-9_006 | | VRQR | Promoter | GAATGAGTGGA | |
| | | | CBE | | | |
| gRNA1759 | ABE_NGA_20nt_ | spCas9 | spCas9 | TTR | CCATCCTGCCAA | 939 |
| | 3-12_019 | | VRQR | Promoter | GAATGAGTGGA | |
| | | | IBE | | | |
| gRNA1760 | ABE_NNNRRT_ | saCas9 | saCas9 | TTR | CCTGCTGATTCT | 940 |
| | 21nt_5-14_014 | | KKH | Promoter | GATTATTGACTT | |
| | | | ABE | | AGT | |
| gRNA1760 | CBE_NNNRRT_ | saCas9 | saCas9 | TTR | CCTGCTGATTCT | 940 |
| | 21nt_3-12_015 | | KKH | Promoter | GATTATTGACTT | |
| | | | CBE | | AGT | |
| gRNA1761 | ABE_NNNRRT_ | saCas9 | saCas9 | TTR | CGATGCTCTAAT | 941 |
| | 21nt_5-14_014 | | KKH | Promoter | CTCTCTAGACAA | |
| | | | ABE | | GGT | |
| gRNA1761 | CBE_NNNRRT_ | saCas9 | saCas9 | TTR | CGATGCTCTAAT | 941 |
| | 21nt_3-12_015 | | KKH | Promoter | CTCTCTAGACAA | |
| | | | CBE | | GGT | |
| gRNA1762 | ABE_NNNRRT_ | saCas9 | saCas9 | TTR | CTAAGTCAATAA | 942 |
| | 21nt_5-14_014 | | KKH | Promoter | TCAGAATCAGCA | |
| | | | ABE | | GGT | |
| gRNA1762 | CBE_NNNRRT_ | saCas9 | saCas9 | TTR | CTAAGTCAATAA | 942 |
| | 21nt_3-12_015 | | KKH | Promoter | TCAGAATCAGCA | |
| | | | CBE | | GGT | |
| gRNA1763 | ABE_NNGRRT_ | saCas9 | saCas9 | TTR | CTAGACAAGGTT | 943 |
| | 21nt_5-14_011 | | ABE | Promoter | CATATTTGTATG | |
| | | | | | GGT | |
| gRNA1763 | CBE_NNGRRT_ | saCas9 | saCas9 | TTR | CTAGACAAGGTT | 943 |
| | 21nt_3-12_012 | | CBE | Promoter | CATATTTGTATG | |
| | | | | | GGT | |
| gRNA1764 | ABE_NGA_20nt_ | spCas9 | spCas9 | TTR | GAACCTTGTCTA | 944 |
| | 3-9_005 | | VRQR | Promoter | GAGAGATTAGA | |
| | | | ABE | | | |
| gRNA1764 | CBE_NGA_20nt_ | spCas9 | spCas9 | TTR | GAACCTTGTCTA | 944 |
| | 4-9_006 | | VRQR | Promoter | GAGAGATTAGA | |
| | | | CBE | | | |
| gRNA1764 | ABE_NGA_20nt_ | spCas9 | spCas9 | TTR | GAACCTTGTCTA | 944 |
| | 3-12_019 | | VRQR | Promoter | GAGAGATTAGA | |
| | | | IBE | | | |
| gRNA1765 | ABE_NGG_20nt_ | spCas9 | spCas9 | TTR | GAAGTCCACTCA | 945 |
| | 3-9_002 | | ABE | Promoter | TTCTTGGCAGG | |
| gRNA1765 | CBE_NGG_20nt_ | spCas9 | spCas9 | TTR | GAAGTCCACTCA | 945 |
| | 4-9_003 | | CBE | Promoter | TTCTTGGCAGG | |
| gRNA1765 | ABE_NGG_20nt_ | spCas9 | spCas9 | TTR | GAAGTCCACTCA | 945 |
| | 3-12_018 | | IBE | Promoter | TTCTTGGCAGG | |
| gRNA1766 | ABE_NGA_20nt_ | spCas9 | spCas9 | TTR | GAATCAGCAGGT | 946 |
| | 3-9_005 | | VRQR | Promoter | TTGCAGTCAGA | |
| | | | ABE | | | |
| gRNA1766 | CBE_NGA_20nt_ | spCas9 | spCas9 | TTR | GAATCAGCAGGT | 946 |
| | 4-9_006 | | VRQR | Promoter | TTGCAGTCAGA | |
| | | | CBE | | | |
| gRNA1766 | ABE_NGA_20nt_ | spCas9 | spCas9 | TTR | GAATCAGCAGGT | 946 |
| | 3-12_019 | | VRQR | Promoter | TTGCAGTCAGA | |
| | | | IBE | | | |
| gRNA1767 | ABE_NGG_20nt_ | spCas9 | spCas9 | TTR | GAATGAGTGGAC | 947 |
| | 3-9_002 | | ABE | Promoter | TTCTGTGATGG | |
| gRNA1767 | CBE_NGG_20nt_ | spCas9 | spCas9 | TTR | GAATGAGTGGAC | 947 |
| | 4-9_003 | | CBE | Promoter | TTCTGTGATGG | |
| gRNA1767 | ABE_NGG_20nt_ | spCas9 | spCas9 | TTR | GAATGAGTGGAC | 947 |
| | 3-12_018 | | IBE | Promoter | TTCTGTGATGG | |
| gRNA1768 | ABE_NGA_20nt_ | spCas9 | spCas9 | TTR | GACTGCAAACCT | 948 |
| | 3-9_005 | | VRQR | Promoter | GCTGATTCTGA | |
| | | | ABE | | | |
| gRNA1768 | CBE_NGA_20nt_ | spCas9 | spCas9 | TTR | GACTGCAAACCT | 948 |
| | 4-9_006 | | VRQR | Promoter | GCTGATTCTGA | |
| | | | CBE | | | |
| gRNA1768 | ABE_NGA_20nt_ | spCas9 | spCas9 | TTR | GACTGCAAACCT | 948 |
| | 3-12_019 | | VRQR | Promoter | GCTGATTCTGA | |
| | | | IBE | | | |
| gRNA1769 | ABE_NNNRRT_ | saCas9 | saCas9 | TTR | GACTTAGTCAAC | 949 |
| | 21nt_5-14_014 | | KKH | Promoter | AAAGAGAGAATA | |
| | | | ABE | | AGT | |
| gRNA1769 | CBE_NNNRRT_ | saCas9 | saCas9 | TTR | GACTTAGTCAAC | 949 |
| | 21nt_3-12_015 | | KKH | Promoter | AAAGAGAGAATA | |
| | | | CBE | | AGT | |
| gRNA1770 | ABE_NGA_20nt_ | spCas9 | spCas9 | TTR | GATAAGCAGCCT | 950 |
| | 3-9_005 | | VRQR | Promoter | AGCTCAGGAGA | |
| | | | ABE | | | |
| gRNA1770 | CBE_NGA_20nt_ | spCas9 | spCas9 | TTR | GATAAGCAGCCT | 950 |
| | 4-9_006 | | VRQR | Promoter | AGCTCAGGAGA | |
| | | | CBE | | | |
| gRNA1770 | ABE_NGA_20nt_ | spCas9 | spCas9 | TTR | GATAAGCAGCCT | 950 |
| | 3-12_019 | | VRQR | Promoter | AGCTCAGGAGA | |
| | | | IBE | | | |
| gRNA1771 | ABE_NGA_20nt_ | spCas9 | spCas9 | TTR | GATGAGAAGCCA | 951 |
| | 3-9_005 | | VRQR | Promoter | TCCTGCCAAGA | |
| | | | ABE | | | |
| gRNA1771 | CBE_NGA_20nt_ | spCas9 | spCas9 | TTR | GATGAGAAGCCA | 951 |
| | 4-9_006 | | VRQR | Promoter | TCCTGCCAAGA | |
| | | | CBE | | | |
| gRNA1771 | ABE_NGA_20nt_ | spCas9 | spCas9 | TTR | GATGAGAAGCCA | 951 |
| | 3-12_019 | | VRQR | Promoter | TCCTGCCAAGA | |
| | | | IBE | | | |
| gRNA1772 | ABE_NGG_20nt_ | spCas9 | spCas9 | TTR | GCCATCCTGCCA | 952 |
| | 3-9_002 | | ABE | Promoter | AGAATGAGTGG | |
| gRNA1772 | CBE_NGG_20nt_ | spCas9 | spCas9 | TTR | GCCATCCTGCCA | 952 |
| | 4-9_003 | | CBE | Promoter | AGAATGAGTGG | |
| gRNA1772 | ABE_NGG_20nt_ | spCas9 | spCas9 | TTR | GCCATCCTGCCA | 952 |
| | 3-12_018 | | IBE | Promoter | AGAATGAGTGG | |
| gRNA1773 | ABE_NGA_20nt_ | spCas9 | spCas9 | TTR | GCTTTTATACTC | 953 |
| | 3-9_005 | | VRQR | Promoter | ACTTCTCCTGA | |
| | | | ABE | | | |
| gRNA1773 | CBE_NGA_20nt_ | spCas9 | spCas9 | TTR | GCTTTTATACTC | 953 |
| | 4-9_006 | | VRQR | Promoter | ACTTCTCCTGA | |
| | | | CBE | | | |
| gRNA1773 | ABE_NGA_20nt_ | spCas9 | spCas9 | TTR | GCTTTTATACTC | 953 |
| | 3-12_019 | | VRQR | Promoter | ACTTCTCCTGA | |
| | | | IBE | | | |
| gRNA1774 | ABE_NNNRRT_ | saCas9 | saCas9 | TTR | GGATAAGCAGCC | 954 |
| | 21nt_5-14_014 | | KKH | Promoter | TAGCTCAGGAGA | |
| | _ | | ABE | | AGT | |
| gRNA1774 | CBE_NNNRRT_ | saCas9 | saCas9 | TTR | GGATAAGCAGCC | 954 |
| | 21nt_3-12_015 | | KKH | Promoter | TAGCTCAGGAGA | |
| | | | CBE | | AGT | |
| gRNA1775 | ABE_NGG_20nt_ | spCas9 | spCas9 | TTR | GTCTAGAGAGAT | 955 |
| | 3-9_002 | | ABE | Promoter | TAGAGCATCGG | |
| gRNA1775 | CBE_NGG_20nt_ | spCas9 | spCas9 | TTR | GTCTAGAGAGAT | 955 |
| | 4-9_003 | | CBE | Promoter | TAGAGCATCGG | |
| gRNA1775 | ABE_NGG_20nt_ | spCas9 | spCas9 | TTR | GTCTAGAGAGAT | 955 |
| | 3-12_018 | | IBE | Promoter | TAGAGCATCGG | |
| gRNA1776 | ABE_NGG_20nt_ | spCas9 | spCas9 | TTR | GTGATGGCTGCT | 956 |
| | 3-9_002 | | ABE | Promoter | CCCAGCCTGGG | |
| gRNA1776 | CBE_NGG_20nt_ | spCas9 | spCas9 | TTR | GTGATGGCTGCT | 956 |
| | 4-9_003 | | CBE | Promoter | CCCAGCCTGGG | |
| gRNA1776 | ABE_NGG_20nt_ | spCas9 | spCas9 | TTR | GTGATGGCTGCT | 956 |
| | 3-12_018 | | IBE | Promoter | CCCAGCCTGGG | |
| gRNA1777 | ABE_NNNRRT_ | saCas9 | saCas9 | TTR | TACTTATTCTCT | 957 |
| | 21nt_5-14_014 | | KKH | Promoter | CTTTGTTGACTA | |
| | | | ABE | | AGT | |
| gRNA1777 | CBE_NNNRRT_ | saCas9 | saCas9 | TTR | TACTTATTCTCT | 957 |
| | 21nt_3-12_015 | | KKH | Promoter | CTTTGTTGACTA | |
| | | | CBE | | AGT | |
| gRNA1778 | ABE_NNNRRT_ | saCas9 | saCas9 | TTR | TATTCTCTCTTT | 958 |
| | 21nt_5-14_014 | | KKH | Promoter | GTTGACTAAGTC | |
| | | | ABE | | AAT | |
| gRNA1778 | CBE_NNNRRT_ | saCas9 | saCas9 | TTR | TATTCTCTCTTT | 958 |
| | 21nt_3-12_015 | | KKH | Promoter | GTTGACTAAGTC | |
| | | | CBE | | AAT | |
| gRNA1779 | ABE_NGA_20nt_ | spCas9 | spCas9 | TTR | TATTGACTTAGT | 959 |
| | 3-9_005 | | VRQR | Promoter | CAACAAAGAGA | |
| | | | ABE | | | |
| gRNA1779 | CBE_NGA_20nt_ | spCas9 | spCas9 | TTR | TATTGACTTAGT | 959 |
| | 4-9_006 | | VRQR | Promoter | CAACAAAGAGA | |
| | | | CBE | | | |
| gRNA1779 | ABE_NGA_20nt_ | spCas9 | spCas9 | TTR | TATTGACTTAGT | 959 |
| | 3-12_019 | | VRQR | Promoter | CAACAAAGAGA | |
| | | | IBE | | | |
| gRNA1780 | ABE_NNGRRT_ | saCas9 | saCas9 | TTR | TATTGACTTAGT | 960 |
| | 21nt_5-14_011 | | ABE | Promoter | CAACAAAGAGAG | |
| | | | | | AAT | |
| gRNA1780 | CBE_NNGRRT_ | saCas9 | saCas9 | TTR | TATTGACTTAGT | 960 |
| | 21nt_3-12_012 | | CBE | Promoter | CAACAAAGAGAG | |
| | | | | | AAT | |
| gRNA1781 | ABE_NNNRRT_ | saCas9 | saCas9 | TTR | TCAGAATCAGCA | 961 |
| | 21nt_5-14_014 | | KKH | Promoter | GGTTTGCAGTCA | |
| | | | ABE | | GAT | |
| gRNA1781 | CBE_NNNRRT | saCas9 | saCas9 | TTR | TCAGAATCAGCA | 961 |
| | 21nt_3-12_015 | | KKH | Promoter | GGTTTGCAGTCA | |
| | | | CBE | | GAT | |
| gRNA1782 | ABE_NGG_20nt_ | spCas9 | spCas9 | TTR | TCCACTCATTCT | 962 |
| | 3-9_002 | | ABE | Promoter | TGGCAGGATGG | |
| gRNA1782 | CBE_NGG_20nt_ | spCas9 | spCas9 | TTR | TCCACTCATTCT | 962 |
| | 4-9_003 | | CBE | Promoter | TGGCAGGATGG | |
| gRNA1782 | ABE_NGG_20nt_ | spCas9 | spCas9 | TTR | TCCACTCATTCT | 962 |
| | 3-12_018 | | IBE | Promoter | TGGCAGGATGG | |
| gRNA1783 | ABE_NNNRRT_ | saCas9 | saCas9 | TTR | TCTCTCTTTGTT | 963 |
| | 21nt_5-14_014 | | KKH | Promoter | GACTAAGTCAAT | |
| | | | ABE | | AAT | |
| gRNA1783 | CBE_NNNRRT_ | saCas9 | saCas9 | TTR | TCTCTCTTTGTT | 963 |
| | 21nt_3-12_015 | | KKH | Promoter | GACTAAGTCAAT | |
| | | | CBE | | AAT | |
| gRNA1784 | ABE_NNGRRT_ | saCas9 | saCas9 | TTR | TGAGAAGCCATC | 964 |
| | 21nt_5-14_011 | | ABE | Promoter | CTGCCAAGAATG | |
| | | | | | AGT | |
| gRNA1784 | CBE_NNGRRT_ | saCas9 | saCas9 | TTR | TGAGAAGCCATC | 964 |
| | 21nt_3-12_012 | | CBE | Promoter | CTGCCAAGAATG | |
| | | | | | AGT | |
| gRNA1785 | ABE_NNNRRT_ | saCas9 | saCas9 | TTR | TGAGCTAGGCTG | 965 |
| | 21nt_5-14_014 | | KKH | Promoter | CTTATCCCTGCC | |
| | | | ABE | | AAT | |
| gRNA1785 | CBE_NNNRRT_ | saCas9 | saCas9 | TTR | TGAGCTAGGCTG | 965 |
| | 21nt_3-12_015 | | KKH | Promoter | CTTATCCCTGCC | |
| | | | CBE | | AAT | |
| gRNA1786 | ABE_NGG_20nt_ | spCas9 | spCas9 | TTR | TGAGTATAAAAG | 966 |
| | 3-9_002 | | ABE | Promoter | CCCCAGGCTGG | |
| gRNA1786 | CBE_NGG_20nt_ | spCas9 | spCas9 | TTR | TGAGTATAAAAG | 966 |
| | 4-9_003 | | CBE | Promoter | CCCCAGGCTGG | |
| gRNA1786 | ABE_NGG_20nt_ | spCas9 | spCas9 | TTR | TGAGTATAAAAG | 966 |
| | 3-12_018 | | IBE | Promoter | CCCCAGGCTGG | |
| gRNA1787 | ABE_NGG_20nt_ | spCas9 | spCas9 | TTR | TGATGGCTGCTC | 967 |
| | 3-9_002 | | ABE | Promoter | CCAGCCTGGGG | |
| gRNA1787 | CBE_NGG_20nt_ | spCas9 | spCas9 | TTR | TGATGGCTGCTC | 967 |
| | 4-9_003 | | CBE | Promoter | CCAGCCTGGGG | |
| gRNA1787 | ABE_NGG_20nt_ | spCas9 | spCas9 | TTR | TGATGGCTGCTC | 967 |
| | 3-12_018 | | IBE | Promoter | CCAGCCTGGGG | |
| gRNA1788 | ABE_NNNRRT_ | saCas9 | saCas9 | TTR | TGCCAAGAATGA | 968 |
| | 21nt_5-14_014 | | KKH | Promoter | GTGGACTTCTGT | |
| | | | ABE | | GAT | |
| gRNA1788 | CBE_NNNRRT_ | saCas9 | saCas9 | TTR | TGCCAAGAATGA | 968 |
| | 21nt_3-12_015 | | KKH | Promoter | GTGGACTTCTGT | |
| | | | CBE | | GAT | |
| gRNA1789 | ABE_NNNRRT_ | saCas9 | saCas9 | TTR | TGCCAATCTGAC | 969 |
| | 21nt_5-14_014 | | KKH | Promoter | TGCAAACCTGCT | |
| | | | ABE | TTR | GAT | |
| gRNA1789 | CBE_NNNRRT_ | saCas9 | saCas9 | Promoter | TGCCAATCTGAC | 969 |
| | 21nt_3-12_015 | | KKH | | TGCAAACCTGCT | |
| | | | CBE | | GAT | |
| gRNA1790 | ABE_NGA_20nt_ | spCas9 | spCas9 | TTR | TGTTGACTAAGT | 970 |
| | 3-9_005 | | VRQR | Promoter | CAATAATCAGA | |
| | | | ABE | | | |
| gRNA1790 | CBE_NGA_20nt_ | spCas9 | spCas9 | TTR | TGTTGACTAAGT | 970 |
| | 4-9_006 | | VRQR | Promoter | CAATAATCAGA | |
| | | | CBE | | | |
| gRNA1790 | ABE_NGA_20nt_ | spCas9 | spCas9 | TTR | TGTTGACTAAGT | 970 |
| | 3-12_019 | | VRQR | Promoter | CAATAATCAGA | |
| | | | IBE | | | |
| gRNA1791 | ABE_NGA_20nt_ | spCas9 | spCas9 | TTR | TTGACTTAGTCA | 971 |
| | 3-9_005 | | VRQR | Promoter | ACAAAGAGAGA | |
| | | | ABE | | | |
| gRNA1791 | CBE_NGA_20nt_ | spCas9 | spCas9 | TTR | TTGACTTAGTCA | 971 |
| | 4-9_006 | | VRQR | Promoter | ACAAAGAGAGA | |
| | | | CBE | | | |
| gRNA1791 | ABE_NGA_20nt_ | spCas9 | spCas9 | TTR | TTGACTTAGTCA | 971 |
| | 3-12_019 | | VRQR | Promoter | ACAAAGAGAGA | |
| | | | IBE | | | |
| gRNA1792 | ABE_NNGRRT_ | saCas9 | saCas9 | TTR | TTTGTTGACTAA | 972 |
| | 21nt_5-14_011 | | ABE | Promoter | GTCAATAATCAG | |
| | | | | | AAT | |
| gRNA1792 | CBE_NNGRRT_ | saCas9 | saCas9 | TTR | TTTGTTGACTAA | 972 |
| | 21nt_3-12_012 | | CBE | Promoter | GTCAATAATCAG | |
| | | | | | AAT | |
| gRNA-#1 | ABE_NGC_20nt_ | spCas9 | spCas9 | TTR | AAAAGCCCCAGG | 973 |
| | 3-9_008 | | NGC | Promoter | CTGGGAGCAGC | |
| | | | ABE | | | |
| gRNA-#1 | CBE_NGC_20nt_ | spCas9 | spCas9 | TTR | AAAAGCCCCAGG | 973 |
| | 4-9_009 | | NGC | Promoter | CTGGGAGCAGC | |
| | | | CBE | | | |
| gRNA-#1 | ABE_NGC_20nt_ | spCas9 | spCas9 | TTR | AAAAGCCCCAGG | 973 |
| | 3-12_020 | | NGC IBE | Promoter | CTGGGAGCAGC | |
| gRNA-#2 | ABE_NGC_20nt_ | | spCas9 | TTR | AAGTGAGTATAA | 974 |
| | 3-9_008 | | NGC | | AAGCCCCAGGC | |
| gRNA-#2 | CBE_NGC_20nt_ | spCas9 | ABE | Promoter | AAGTGAGTATAA | 974 |
| | 4-9_009 | spCas9 | spCas9 | TTR | AAGCCCCAGGC | |
| | | | NGC | | | |
| gRNA-#2 | ABE_NGC_20nt_ | | CBE | Promoter | AAGTGAGTATAA | 974 |
| | 3-12_020 | spCas9 | spCas9 | TTR | AAGCCCCAGGC | |
| gRNA-#3 | ABE_NNNRRT_ | saCas9 | NGC IBE | Promoter | AATAATCAGAAT | 975 |
| | 21nt_5-14_014 | | saCas9 | TTR | CAGCAGGTTTGC | |
| | | | KKH | Promoter | AGT | |
| | | | ABE | | | |
| gRNA-#3 | CBE_NNNRRT_ | saCas9 | saCas9 | TTR | AATAATCAGAAT | 975 |
| | 21nt_3-12_015 | | KKH | Promoter | CAGCAGGTTTGC | |
| | | | CBE | | AGT | |
| gRNA-#4 | CBE_NGA_20nt_ | spCas9 | spCas9 | TTR | AATATGAACCTT | 976 |
| | 4-9_006 | | VRQR | Promoter | GTCTAGAGAGA | |
| | | | CBE | | | |
| gRNA-#4 | ABE_NGA_20nt_ | spCas9 | spCas9 | TTR | AATATGAACCTT | 976 |
| | 3-9_005 | | VRQR | Promoter | GTCTAGAGAGA | |
| | | | ABE | | | |
| gRNA-#4 | ABE_NGA_20nt_ | spCas9 | spCas9 | TTR | AATATGAACCTT | 976 |
| | 3-12_019 | | VRQR | Promoter | GTCTAGAGAGA | |
| | | | IBE | | | |
| gRNA-#5 | ABE_NGC_20nt_ | spCas9 | spCas9 | TTR | AATGAGTGGACT | 977 |
| | 3-9_008 | | NGC | Promoter | TCTGTGATGGC | |
| | | | ABE | | | |
| gRNA-#5 | CBE_NGC_20nt_ | spCas9 | spCas9 | TTR | AATGAGTGGACT | 977 |
| | 4-9_009 | | NGC | Promoter | TCTGTGATGGC | |
| | | | CBE | | | |
| gRNA-#5 | ABE_NGC_20nt_ | spCas9 | spCas9 | TTR | AATGAGTGGACT | 977 |
| | 3-12_020 | | NGC IBE | Promoter | TCTGTGATGGC | |
| gRNA-#6 | ABE_NNNRRT | saCas9 | saCas9 | TTR | ACAAATATGAAC | 978 |
| | 21nt_5-14_014 | | KKH | Promoter | CTTGTCTAGAGA | |
| | | | ABE | | GAT | |
| gRNA-#6 | CBE_NNNRRT | saCas9 | saCas9 | TTR | ACAAATATGAAC | 978 |
| | 21nt_3-12_015 | | KKH | Promoter | CTTGTCTAGAGA | |
| | | | CBE | | GAT | |
| gRNA-#7 | ABE_NGC_20nt_ | spCas9 | spCas9 | TTR | ACAGAAGTCCAC | 979 |
| | 3-9_008 | | NGC | Promoter | TCATTCTTGGC | |
| | | | ABE | | | |
| gRNA-#7 | CBE_NGC_20nt_ | spCas9 | spCas9 | TTR | ACAGAAGTCCAC | 979 |
| | 4-9_009 | | NGC | Promoter | TCATTCTTGGC | |
| | | | CBE | | | |
| gRNA-#7 | ABE_NGC_20nt_ | spCas9 | spCas9 | TTR | ACAGAAGTCCAC | 979 |
| | 3-12_020 | | NGC IBE | Promoter | TCATTCTTGGC | |
| gRNA-#8 | ABE_NGC_20nt_ | spCas9 | spCas9 | TTR | ACCTTGTCTAGA | 980 |
| | 3-9_008 | | NGC | Promoter | GAGATTAGAGC | |
| | | | ABE | | | |
| gRNA-#8 | CBE_NGC_20nt_ | spCas9 | spCas9 | TTR | ACCTTGTCTAGA | 980 |
| | 4-9_009 | | NGC | Promoter | GAGATTAGAGC | |
| | | | CBE | | | |
| gRNA-#8 | ABE_NGC_20nt_ | spCas9 | spCas9 | TTR | ACCTTGTCTAGA | 980 |
| | 3-12_020 | | NGC IBE | Promoter | GAGATTAGAGC | |
| gRNA-#9 | CBE_NGA_20nt_ | spCas9 | spCas9 | TTR | AGAAGCCATCCT | 981 |
| | 4-9_006 | | VRQR | Promoter | GCCAAGAATGA | |
| | | | CBE | | | |
| gRNA-#9 | ABE_NGA_20nt_ | spCas9 | spCas9 | TTR | AGAAGCCATCCT | 981 |
| | 3-9_005 | | VRQR | Promoter | GCCAAGAATGA | |
| | | | ABE | | | |
| gRNA-#9 | ABE_NGA_20nt_ | spCas9 | spCas9 | TTR | AGAAGCCATCCT | 981 |
| | 3-12_019 | | VRQR | Promoter | GCCAAGAATGA | |
| | | | IBE | | | |
| gRNA-#10 | ABE_NGC_20nt_ | spCas9 | spCas9 | TTR | AGCAGGTTTGCA | 982 |
| | 3-9_008 | | NGC | Promoter | GTCAGATTGGC | |
| | | | ABE | | | |
| gRNA-#10 | CBE_NGC_20nt_ | spCas9 | spCas9 | TTR | AGCAGGTTTGCA | 982 |
| | 4-9_009 | | NGC | Promoter | GTCAGATTGGC | |
| | | | CBE | | | |
| gRNA-#10 | ABE_NGC_20nt_ | spCas9 | spCas9 | TTR | AGCAGGTTTGCA | 982 |
| | 3-12_020 | | NGC IBE | Promoter | GTCAGATTGGC | |
| gRNA-#11 | ABE_NGG_20nt_ | spCas9 | spCas9 | TTR | AGGGATAAGCAG | 983 |
| | 3-9_002 | | ABE | Promoter | CCTAGCTCAGG | |
| gRNA-#11 | CBE_NGG_20nt_ | spCas9 | spCas9 | TTR | AGGGATAAGCAG | 983 |
| | 4-9_003 | | CBE | Promoter | CCTAGCTCAGG | |
| gRNA-#11 | ABE_NGG_20nt_ | spCas9 | spCas9 | TTR | AGGGATAAGCAG | 983 |
| | 3-12_018 | | IBE | Promoter | CCTAGCTCAGG | |
| gRNA-#12 | ABE_NGG_20nt_ | spCas9 | spCas9 | TTR | AGGTTTGCAGTC | 984 |
| | 3-9_002 | | ABE | Promoter | AGATTGGCAGG | |
| gRNA-#12 | CBE_NGG_20nt_ | spCas9 | spCas9 | TTR | AGGTTTGCAGTC | 984 |
| | 4-9_003 | | CBE | Promoter | AGATTGGCAGG | |
| gRNA-#12 | ABE_NGG_20nt_ | spCas9 | spCas9 | TTR | AGGTTTGCAGTC | 984 |
| | 3-12_018 | | IBE | Promoter | AGATTGGCAGG | |
| gRNA-#13 | CBE_NGA_20nt_ | spCas9 | spCas9 | TTR | AGTATAAAAGCC | 985 |
| | 4-9_006 | | VRQR | Promoter | CCAGGCTGGGA | |
| | | | CBE | | | |
| gRNA-#13 | ABE_NGA_20nt_ | spCas9 | spCas9 | TTR | AGTATAAAAGCC | 985 |
| | 3-9_005 | | VRQR | Promoter | CCAGGCTGGGA | |
| | | | ABE | | | |
| gRNA-#13 | ABE_NGA_20nt_ | spCas9 | spCas9 | TTR | AGTATAAAAGCC | 985 |
| | 3-12_019 | | VRQR | Promoter | CCAGGCTGGGA | |
| | | | IBE | | | |
| gRNA-#14 | ABE_NGG_20nt_ | spCas9 | spCas9 | TTR | AGTCAATAATCA | 986 |
| | 3-9_002 | | ABE | Promoter | GAATCAGCAGG | |
| gRNA-#14 | CBE_NGG_20nt_ | spCas9 | spCas9 | TTR | AGTCAATAATCA | 986 |
| | 4-9_003 | | CBE | Promoter | GAATCAGCAGG | |
| gRNA-#14 | ABE_NGG_20nt_ | spCas9 | spCas9 | TTR | AGTCAATAATCA | 986 |
| | 3-12_018 | | IBE | Promoter | GAATCAGCAGG | |
| gRNA-#15 | ABE_NGC_20nt_ | spCas9 | spCas9 | TTR | ATAATCAGAATC | 987 |
| | 3-9_008 | | NGC | Promoter | AGCAGGTTTGC | |
| | | | ABE | | | |
| gRNA-#15 | CBE_NGC_20nt_ | spCas9 | spCas9 | TTR | ATAATCAGAATC | 987 |
| | 4-9_009 | | NGC | Promoter | AGCAGGTTTGC | |
| | | | CBE | | | |
| gRNA-#15 | ABE_NGC_20nt_ | spCas9 | spCas9 | TTR | ATAATCAGAATC | 987 |
| | 3-12_020 | | NGC IBE | Promoter | AGCAGGTTTGC | |
| gRNA-#16 | ABE_VTTN_ | cas12b | cas12b | TTR | ATTATTGACTTA | 988 |
| | 22nt_5-9_017 | | ABE | Promoter | GTCAACAAAGAG | |
| | | | | | AG | |
| gRNA-#17 | ABE_VTTN_ | cas12b | cas 12b | TTR | ATTCTCTCTTTG | 989 |
| | 22nt_5-9_017 | | ABE | Promoter | TTGACTAAGTCA | |
| | | | | | AT | |
| gRNA-#18 | ABE_VTTN_ | cas12b | cas12b | TTR | ATTCTGATTATT | 990 |
| | 22nt_5-9_017 | | ABE | Promoter | GACTTAGTCAAC | |
| | | | | | AA | |
| gRNA-#19 | ABE_VTTN_ | cas12b | cas 12b | TTR | ATTCTTGGCAGG | 991 |
| (sgRNA 36 | 22nt_5-9_017 | | ABE | Promoter | ATGGCTTCTCAT | |
| 7) | | | | | CG | |
| gRNA-#20 | ABE_VTTN_ | cas 12b | cas12b | TTR | ATTGACTTAGTC | 992 |
| | 22nt_5-9_017 | | ABE | Promoter | AACAAAGAGAGA | |
| | | | | | AT | |
| gRNA-#21 | ABE_VTTN_ | cas12b | cas12b | TTR | ATTGGCAGGGAT | 993 |
| | 22nt_5-9_017 | | ABE | Promoter | AAGCAGCCTAGC | |
| | | | | | TC | |
| gRNA-#22 | ABE_VTTN_ | cas12b | cas 12b | TTR | ATTTGTATGGGT | 994 |
| | 22nt_5-9_017 | | ABE | Promoter | TACTTATTCTCT | |
| | | | | | CT | |
| gRNA-#23 | CBE_NGA_20nt_ | spCas9 | spCas9 | TTR | CAAGAATGAGTG | 995 |
| | 4-9_006 | | VRQR | Promoter | GACTTCTGTGA | |
| | | | CBE | | | |
| gRNA-#23 | ABE_NGA_20nt_ | spCas9 | spCas9 | TTR | CAAGAATGAGTG | 995 |
| | 3-9_005 | | VRQR | Promoter | GACTTCTGTGA | |
| | | | ABE | | | |
| gRNA-#23 | ABE_NGA_20nt_ | spCas9 | spCas9 | TTR | CAAGAATGAGTG | 995 |
| | 3-12_019 | | VRQR | Promoter | GACTTCTGTGA | |
| | | | IBE | | | |
| gRNA-#24 | CBE_NGA_20nt_ | spCas9 | spCas9 | TTR | CAATCTGACTGC | 996 |
| | 4-9_006 | | VRQR | Promoter | AAACCTGCTGA | |
| | | | CBE | | | |
| gRNA-#24 | ABE_NGA_20nt_ | spCas9 | spCas9 | TTR | CAATCTGACTGC | 996 |
| | 3-9_005 | | VRQR | Promoter | AAACCTGCTGA | |
| | | | ABE | | | |
| gRNA-#24 | ABE_NGA_20nt_ | spCas9 | spCas9 | TTR | CAATCTGACTGC | 996 |
| | 3-12_019 | | VRQR | Promoter | AAACCTGCTGA | |
| | | | IBE | | | |
| gRNA-#25 | ABE_NGG_20nt_ | spCas9 | spCas9 | TTR | CACAGAAGTCCA | 997 |
| | 3-9_002 | | ABE | Promoter | CTCATTCTTGG | |
| gRNA-#25 | CBE_NGG_20nt_ | spCas9 | spCas9 | TTR | CACAGAAGTCCA | 997 |
| | 4-9_003 | | CBE | Promoter | CTCATTCTTGG | |
| gRNA-#25 | ABE_NGG_20nt_ | spCas9 | spCas9 | TTR | CACAGAAGTCCA | 997 |
| | 3-12_018 | | IBE | Promoter | CTCATTCTTGG | |
| gRNA-#26 | ABE_NGC_20nt_ | spCas9 | spCas9 | TTR | CAGACGATGAGA | 998 |
| | 3-9_008 | | NGC | Promoter | AGCCATCCTGC | |
| | | | ABE | | | |
| gRNA-#26 | CBE_NGC_20nt_ | spCas9 | spCas9 | TTR | CAGACGATGAGA | 998 |
| | 4-9_009 | | NGC | Promoter | AGCCATCCTGC | |
| | | | CBE | | | |
| gRNA-#26 | ABE_NGC_20nt_ | spCas9 | spCas9 | TTR | CAGACGATGAGA | 998 |
| | 3-12_020 | | NGC IBE | Promoter | AGCCATCCTGC | |
| gRNA-#27 | ABE_NGG_20nt_ | spCas9 | spCas9 | TTR | CAGCAGGTTTGC | 999 |
| | 3-9_002 | | ABE | Promoter | AGTCAGATTGG | |
| gRNA-#27 | CBE_NGG_20nt_ | spCas9 | spCas9 | TTR | CAGCAGGTTTGC | 999 |
| | 4-9_003 | | CBE | Promoter | AGTCAGATTGG | |
| gRNA-#27 | ABE_NGG_20nt_ | spCas9 | spCas9 | TTR | CAGCAGGTTTGC | 999 |
| | 3-12_018 | | IBE | Promoter | AGTCAGATTGG | |
| gRNA-#28 | ABE_NGC_20nt_ | spCas9 | spCas9 | TTR | CAGGATGGCTTC | 1000 |
| | 3-9_008 | | NGC | Promoter | TCATCGTCTGC | |
| | | | ABE | | | |
| gRNA-#28 | CBE_NGC_20nt_ | spCas9 | spCas9 | TTR | CAGGATGGCTTC | 1000 |
| | 4-9_009 | | NGC | Promoter | TCATCGTCTGC | |
| | | | CBE | | | |
| gRNA-#28 | ABE_NGC_20nt_ | spCas9 | spCas9 | TTR | CAGGATGGCTTC | 1000 |
| | 3-12_020 | | NGC IBE | Promoter | TCATCGTCTGC | |
| gRNA-#29 | ABE_NNGRRT_ | saCas9 | saCas9 | TTR | CAGGTTTGCAGT | 1001 |
| | 21nt_5-14_011 | | ABE | Promoter | CAGATTGGCAGG | |
| | | | | | GAT | |
| gRNA-#29 | CBE_NNGRRT_ | saCas9 | saCas9 | TTR | CAGGTTTGCAGT | 1001 |
| | 21nt_3-12_012 | | CBE | Promoter | CAGATTGGCAGG | |
| | | | | | GAT | |
| gRNA-#30 | ABE_NGC_20nt_ | spCas9 | spCas9 | TTR | CAGTCAGATTGG | 1002 |
| | 3-9_008 | | NGC | Promoter | CAGGGATAAGC | |
| | | | ABE | | | |
| gRNA-#30 | CBE_NGC_20nt_ | spCas9 | spCas9 | TTR | CAGTCAGATTGG | 1002 |
| | 4-9_009 | | NGC | Promoter | CAGGGATAAGC | |
| | | | CBE | | | |
| gRNA-#30 | ABE_NGC_20nt_ | spCas9 | spCas9 | TTR | CAGTCAGATTGG | 1002 |
| | 3-12_020 | | NGC IBE | Promoter | CAGGGATAAGC | |
| gRNA-#31 | ABE_NGC_20nt_ | spCas9 | spCas9 | TTR | CCACTCATTCTT | 1003 |
| | 3-9_008 | | NGC | Promoter | GGCAGGATGGC | |
| | | | ABE | | | |
| gRNA-#31 | CBE_NGC_20nt_ | spCas9 | spCas9 | TTR | CCACTCATTCTT | 1003 |
| | 4-9_009 | | NGC | Promoter | GGCAGGATGGC | |
| | | | CBE | | | |
| gRNA-#31 | ABE_NGC_20nt_ | spCas9 | spCas9 | TTR | CCACTCATTCTT | 1003 |
| | 3-12_020 | | NGC IBE | Promoter | GGCAGGATGGC | |
| gRNA-#32 | ABE_NGC_20nt_ | spCas9 | spCas9 | TTR | CTAAGTCAATAA | 1004 |
| | 3-9_008 | | NGC | Promoter | TCAGAATCAGC | |
| | | | ABE | | | |
| gRNA-#32 | CBE_NGC_20nt_ | spCas9 | spCas9 | TTR | CTAAGTCAATAA | 1004 |
| | 4-9_009 | | NGC | Promoter | TCAGAATCAGC | |
| | | | CBE | | | |
| gRNA-#32 | ABE_NGC_20nt_ | spCas9 | spCas9 | TTR | CTAAGTCAATAA | 1004 |
| | 3-12_020 | | NGC IBE | Promoter | TCAGAATCAGC | |
| gRNA-#33 | ABE_VTTN_ | cas12b | cas12b | TTR | CTTAGTCAACAA | 1005 |
| | 22nt_5-9_017 | | ABE | Promoter | AGAGAGAATAAG | |
| | | | | | TA | |
| gRNA-#34 | ABE_NGC_20nt_ | spCas9 | spCas9 | TTR | CTTATCCCTGCC | 1006 |
| | 3-9_008 | | NGC | Promoter | AATCTGACTGC | |
| | | | ABE | | | |
| gRNA-#34 | CBE_NGC_20nt_ | spCas9 | spCas9 | TTR | CTTATCCCTGCC | 1006 |
| | 4-9_009 | | NGC | Promoter | AATCTGACTGC | |
| | | | CBE | | | |
| gRNA-#34 | ABE_NGC_20nt_ | spCas9 | spCas9 | TTR | CTTATCCCTGCC | 1006 |
| | 3-12_020 | | NGC IBE | Promoter | AATCTGACTGC | |
| gRNA-#35 | ABE_VTTN_ | cas12b | cas12b | TTR | CTTATCCCTGCC | 1007 |
| | 22nt_5-9_017 | | ABE | Promoter | AATCTGACTGCA | |
| | | | | | AA | |
| gRNA-#36 | ABE_VTTN_ | cas12b | cas12b | TTR | CTTATTCTCTCT | 1008 |
| | 22nt_5-9_017 | | ABE | Promoter | TTGTTGACTAAG | |
| | | | | | TC | |
| gRNA-#37 | ABE_VTTN_ | cas12b | cas12b | TTR | CTTCTCCTGAGC | 1009 |
| | 22nt_5-9_017 | | ABE | Promoter | TAGGCTGCTTAT | |
| | | | | | CC | |
| gRNA-#38 | ABE_NGC_20nt_ | spCas9 | spCas9 | TTR | CTTCTGTGATGG | 1010 |
| | 3-9_008 | | NGC | Promoter | CTGCTCCCAGC | |
| | | | ABE | | | |
| gRNA-#38 | CBE_NGC_20nt_ | spCas9 | spCas9 | TTR | CTTCTGTGATGG | 1010 |
| | 4-9_009 | | NGC | Promoter | CTGCTCCCAGC | |
| | | | CBE | | | |
| gRNA-#38 | ABE_NGC_20nt_ | spCas9 | spCas9 | TTR | CTTCTGTGATGG | 1010 |
| | 3-12_020 | | NGC IBE | Promoter | CTGCTCCCAGC | |
| gRNA-#39 | ABE_VTTN_ | cas12b | cas 12b | TTR | CTTCTGTGATGG | 1011 |
| | 22nt_5-9_017 | | ABE | Promoter | CTGCTCCCAGCC | |
| | | | | | TG | |
| gRNA-#40 | ABE_VTTN_ | cas12b | cas12b | TTR | CTTGGCAGGATG | 1012 |
| | 22nt_5-9_017 | | ABE | Promoter | GCTTCTCATCGT | |
| | | | | | CT | |
| gRNA-#41 | ABE_VTTN_ | cas12b | cas12b | TTR | CTTGTCTAGAGA | 1013 |
| | 22nt_5-9_017 | | ABE | Promoter | GATTAGAGCATC | |
| | | | | | GG | |
| gRNA-#42 | ABE_VTTN_ | cas12b | cas12b | TTR | CTTTGTTGACTA | 1014 |
| | 22nt_5-9_017 | | ABE | Promoter | AGTCAATAATCA | |
| | | | | | GA | |
| gRNA-#43 | ABE_VTTN_ | cas12b | cas12b | TTR | CTTTTATACTCA | 1015 |
| | 22nt_5-9_017 | | ABE | Promoter | CTTCTCCTGAGC | |
| | | | | | TA | |
| gRNA-#44 | ABE_NGG_20nt_ | spCas9 | spCas9 | TTR | GAAGTGAGTATA | 1016 |
| | 3-9_002 | | ABE | Promoter | AAAGCCCCAGG | |
| gRNA-#44 | CBE_NGG_20nt_ | spCas9 | spCas9 | TTR | GAAGTGAGTATA | 1016 |
| | 4-9_003 | | CBE | Promoter | AAAGCCCCAGG | |
| gRNA-#44 | ABE_NGG_20nt_ | spCas9 | spCas9 | TTR | GAAGTGAGTATA | 1016 |
| | 3-12_018 | | IBE | Promoter | AAAGCCCCAGG | |
| gRNA-#45 | ABE_NGG_20nt_ | spCas9 | spCas9 | TTR | GACAAGGTTCAT | 1017 |
| | 3-9_002 | | ABE | Promoter | ATTTGTATGGG | |
| gRNA-#45 | CBE_NGG_20nt_ | spCas9 | spCas9 | TTR | GACAAGGTTCAT | 1017 |
| | 4-9_003 | | CBE | Promoter | ATTTGTATGGG | |
| gRNA-#45 | ABE_NGG_20nt_ | spCas9 | spCas9 | TTR | GACAAGGTTCAT | 1017 |
| | 3-12_018 | | IBE | Promoter | ATTTGTATGGG | |
| gRNA-#46 | ABE_NGG_20nt_ | spCas9 | spCas9 | TTR | GAGTATAAAAGC | 1018 |
| | 3-9_002 | | ABE | Promoter | CCCAGGCTGGG | |
| gRNA-#46 | CBE_NGG_20nt_ | spCas9 | spCas9 | TTR | GAGTATAAAAGC | 1018 |
| | 4-9_003 | | CBE | Promoter | CCCAGGCTGGG | |
| gRNA-#46 | ABE_NGG_20nt_ | spCas9 | spCas9 | TTR | GAGTATAAAAGC | 1018 |
| | 3-12_018 | | IBE | Promoter | CCCAGGCTGGG | |
| gRNA-#47 | ABE_NGC_20nt_ | spCas9 | spCas9 | TTR | GAGTGGACTTCT | 1019 |
| | 3-9_008 | | NGC | Promoter | GTGATGGCTGC | |
| | | | ABE | | | |
| gRNA-#47 | CBE_NGC_20nt_ | spCas9 | spCas9 | TTR | GAGTGGACTTCT | 1019 |
| | 4-9_009 | | NGC | Promoter | GTGATGGCTGC | |
| | | | CBE | | | |
| gRNA-#47 | ABE_NGC_20nt_ | spCas9 | spCas9 | TTR | GAGTGGACTTCT | 1019 |
| | 3-12_020 | | NGC IBE | Promoter | GTGATGGCTGC | |
| gRNA-#48 | ABE_NGC_20nt_ | spCas9 | spCas9 | TTR | GATGGCTGCTCC | 1020 |
| | 3-9_008 | | NGC | Promoter | CAGCCTGGGGC | |
| | | | ABE | | | |
| gRNA-#48 | CBE_NGC_20nt_ | spCas9 | spCas9 | TTR | GATGGCTGCTCC | 1020 |
| | 4-9_009 | | NGC | Promoter | CAGCCTGGGGC | |
| | | | CBE | | | |
| gRNA-#48 | ABE_NGC_20nt_ | spCas9 | spCas9 | TTR | GATGGCTGCTCC | 1020 |
| | 3-12_020 | | NGC IBE | Promoter | CAGCCTGGGGC | |
| gRNA-#48 | CBE_NGA_20nt_ | spCas9 | spCas9 | TTR | GCAGCCTAGCTC | 1021 |
| | 4-9_006 | | VRQR | Promoter | AGGAGAAGTGA | |
| | | | CBE | | | |
| gRNA-#48 | ABE_NGA_20nt_ | spCas9 | spCas9 | TTR | GCAGCCTAGCTC | 1021 |
| | 3-9_005 | | VRQR | Promoter | AGGAGAAGTGA | |
| | | | ABE | | | |
| gRNA-#48 | ABE_NGA_20nt_ | spCas9 | spCas9 | TTR | GCAGCCTAGCTC | 1021 |
| | 3-12_019 | | VRQR | Promoter | AGGAGAAGTGA | |
| | | | IBE | | | |
| gRNA-#50 | CBE_NGA_20nt_ | spCas9 | spCas9 | TTR | GCTGCTTATCCC | 1022 |
| | 4-9_006 | | VRQR | Promoter | TGCCAATCTGA | |
| | | | CBE | | | |
| gRNA-#50 | ABE_NGA_20nt_ | spCas9 | spCas9 | TTR | GCTGCTTATCCC | 1022 |
| | 3-9_005 | | VRQR | Promoter | TGCCAATCTGA | |
| | | | ABE | | | |
| gRNA-#50 | ABE_NGA_20nt_ | spCas9 | spCas9 | TTR | GCTGCTTATCCC | 1022 |
| | 3-12_019 | | VRQR | Promoter | TGCCAATCTGA | |
| | | | IBE | | | |
| gRNA-#51 | CBE_NGA_20nt_ | spCas9 | spCas9 | TTR | GGGATAAGCAGC | 1023 |
| | 4-9_006 | | VRQR | Promoter | CTAGCTCAGGA | |
| | | | CBE | | | |
| gRNA-#51 | ABE_NGA_20nt_ | spCas9 | spCas9 | TTR | GGGATAAGCAGC | 1023 |
| | 3-9_005 | | VRQR | Promoter | CTAGCTCAGGA | |
| | | | ABE | | | |
| gRNA-#51 | ABE_NGA_20nt_ | spCas9 | spCas9 | TTR | GGGATAAGCAGC | 1023 |
| | 3-12_019 | | VRQR | Promoter | CTAGCTCAGGA | |
| | | | IBE | | | |
| gRNA-#52 | ABE_NGG_20nt_ | spCas9 | spCas9 | TTR | GGTTTGCAGTCA | 1024 |
| | 3-9_002 | | ABE | Promoter | GATTGGCAGGG | |
| gRNA-#52 | CBE_NGG_20nt_ | spCas9 | spCas9 | TTR | GGTTTGCAGTCA | 1024 |
| | 4-9_003 | | CBE | Promoter | GATTGGCAGGG | |
| gRNA-#52 | ABE_NGG_20nt_ | spCas9 | spCas9 | TTR | GGTTTGCAGTCA | 1024 |
| | 3-12_018 | | IBE | Promoter | GATTGGCAGGG | |
| gRNA-#53 | CBE_NGA_20nt_ | spCas9 | spCas9 | TTR | GTTACTTATTCT | 1025 |
| | 4-9_006 | | VRQR | Promoter | CTCTTTGTTGA | |
| | | | CBE | | | |
| gRNA-#53 | ABE_NGA_20nt_ | spCas9 | spCas9 | TTR | GTTACTTATTCT | 1025 |
| | 3-9_005 | | VRQR | Promoter | CTCTTTGTTGA | |
| | | | ABE | | | |
| gRNA-#53 | ABE_NGA_20nt_ | spCas9 | spCas9 | TTR | GTTACTTATTCT | 1025 |
| | 3-12_019 | | VRQR | Promoter | CTCTTTGTTGA | |
| | | | IBE | | | |
| gRNA-#54 | ABE_VTTN_ | cas12b | cas12b | TTR | GTTACTTATTCT | 1026 |
| | 22nt_5-9_017 | | ABE | Promoter | CTCTTTGTTGAC | |
| | | | | | TA | |
| gRNA-#55 | ABE_VTTN_ | cas12b | cas12b | TTR | GTTCATATTTGT | 1027 |
| | 22nt_5-9_017 | | ABE | Promoter | ATGGGTTACTTA | |
| | | | | | TT | |
| gRNA-#56 | ABE_VTTN_ | cas12b | cas12b | TTR | GTTGACTAAGTC | 1028 |
| | 22nt_5-9_017 | | ABE | Promoter | AATAATCAGAAT | |
| | | | | | CA | |
| gRNA-#57 | CBE_NGA_20nt_ | spCas9 | spCas9 | TTR | GTTTGCAGTCAG | 1029 |
| | 4-9_006 | | VRQR | Promoter | ATTGGCAGGGA | |
| | | | CBE | | | |
| gRNA-#57 | ABE_NGA_20nt_ | spCas9 | spCas9 | TTR | GTTTGCAGTCAG | 1029 |
| | 3-9_005 | | VRQR | Promoter | ATTGGCAGGGA | |
| | | | ABE | | | |
| gRNA-#57 | ABE_NGA_20nt_ | spCas9 | spCas9 | TTR | GTTTGCAGTCAG | 1029 |
| | 3-12_019 | | VRQR | Promoter | ATTGGCAGGGA | |
| | | | IBE | | | |
| gRNA-#58 | ABE_VTTN_ | cas12b | cas12b | TTR | GTTTGCAGTCAG | 1030 |
| | 22nt_5-9_017 | | ABE | Promoter | ATTGGCAGGGAT | |
| | | | | | AA | |
| gRNA-#59 | CBE_NGA_20nt_ | spCas9 | spCas9 | TTR | TACAAATATGAA | 1031 |
| | 4-9_006 | | VRQR | Promoter | CCTTGTCTAGA | |
| | | | CBE | | | |
| gRNA-#59 | ABE_NGA_20nt_ | spCas9 | spCas9 | TTR | TACAAATATGAA | 1031 |
| | 3-9_005 | | VRQR | Promoter | CCTTGTCTAGA | |
| | | | ABE | | | |
| gRNA-#59 | ABE_NGA_20nt_ | spCas9 | spCas9 | TTR | TACAAATATGAA | 1031 |
| | 3-12_019 | | VRQR | Promoter | CCTTGTCTAGA | |
| | | | IBE | | | |
| gRNA-#60 | ABE_NGC_20nt_ | spCas9 | spCas9 | TTR | TACTCACTTCTC | 1032 |
| | 3-9_008 | | NGC | Promoter | CTGAGCTAGGC | |
| | | | ABE | | | |
| gRNA-#60 | CBE_NGC_20nt_ | spCas9 | spCas9 | TTR | TACTCACTTCTC | 1032 |
| | 4-9_009 | | NGC | Promoter | CTGAGCTAGGC | |
| | | | CBE | | | |
| gRNA-#60 | ABE_NGC_20nt_ | spCas9 | spCas9 | TTR | TACTCACTTCTC | 1032 |
| | 3-12_020 | | NGC IBE | Promoter | CTGAGCTAGGC | |
| gRNA-#61 | ABE_NGC_20nt_ | spCas9 | spCas9 | TTR | TATAAAAGCCCC | 1033 |
| | 3-9_008 | | NGC | Promoter | AGGCTGGGAGC | |
| | | | ABE | | | |
| gRNA-#61 | CBE_NGC_20nt_ | spCas9 | spCas9 | TTR | TATAAAAGCCCC | 1033 |
| | 4-9_009 | | NGC | Promoter | AGGCTGGGAGC | |
| | | | CBE | | | |
| gRNA-#61 | ABE_NGC_20nt_ | spCas9 | spCas9 | TTR | TATAAAAGCCCC | 1033 |
| | 3-12_020 | | NGC IBE | Promoter | AGGCTGGGAGC | |
| gRNA-#62 | ABE_NGC_20nt_ | spCas9 | spCas9 | TTR | TCACTTCTCCTG | 1034 |
| | 3-9_008 | | NGC | Promoter | AGCTAGGCTGC | |
| | | | ABE | | | |
| gRNA-#62 | CBE_NGC_20nt_ | spCas9 | spCas9 | TTR | TCACTTCTCCTG | 1034 |
| | 4-9_009 | | NGC | Promoter | AGCTAGGCTGC | |
| | | | CBE | | | |
| gRNA-#62 | ABE_NGC_20nt_ | spCas9 | spCas9 | TTR | TCACTTCTCCTG | 1034 |
| | 3-12_020 | | NGC IBE | Promoter | AGCTAGGCTGC | |
| gRNA-#63 | ABE_NGC_20nt_ | spCas9 | spCas9 | TTR | TCAGATTGGCAG | 1035 |
| | 3-9_008 | | NGC | Promoter | GGATAAGCAGC | |
| | | | ABE | | | |
| gRNA-#63 | CBE_NGC_20nt_ | spCas9 | spCas9 | TTR | TCAGATTGGCAG | 1035 |
| | 4-9_009 | | NGC | Promoter | GGATAAGCAGC | |
| | | | CBE | | | |
| gRNA-#63 | ABE_NGC_20nt_ | spCas9 | spCas9 | TTR | TCAGATTGGCAG | 1035 |
| | 3-12_020 | | NGC IBE | Promoter | GGATAAGCAGC | |
| gRNA-#64 | ABE_NGC_20nt_ | spCas9 | spCas9 | TTR | TCAGGAGAAGTG | 1036 |
| | 3-9_008 | | NGC | Promoter | AGTATAAAAGC | |
| | | | ABE | | | |
| gRNA-#64 | CBE_NGC_20nt_ | spCas9 | spCas9 | TTR | TCAGGAGAAGTG | 1036 |
| | 4-9_009 | | NGC | Promoter | AGTATAAAAGC | |
| | | | CBE | | | |
| gRNA-#64 | ABE_NGC_20nt_ | spCas9 | spCas9 | TTR | TCAGGAGAAGTG | 1036 |
| | 3-12_020 | | NGC IBE | Promoter | AGTATAAAAGC | |
| gRNA-#65 | ABE_NNNRRT_ | saCas9 | saCas9 | TTR | TCTGACTGCAAA | 1037 |
| | 21nt_5-14_014 | | KKH | Promoter | CCTGCTGATTCT | |
| | | | ABE | | GAT | |
| gRNA-#65 | CBE_NNNRRT_ | saCas9 | saCas9 | TTR | TCTGACTGCAAA | 1037 |
| | 21nt_3-12_015 | | KKH | Promoter | CCTGCTGATTCT | |
| | | | CBE | | GAT | |
| gRNA-#66 | ABE_NGC_20nt_ | spCas9 | spCas9 | TTR | TGAGCTAGGCTG | 1038 |
| | 3-9_008 | | NGC | Promoter | CTTATCCCTGC | |
| | | | ABE | | | |
| gRNA-#66 | CBE_NGC_20nt_ | spCas9 | spCas9 | TTR | TGAGCTAGGCTG | 1038 |
| | 4-9_009 | | NGC | Promoter | CTTATCCCTGC | |
| | | | CBE | | | |
| gRNA-#66 | ABE_NGC_20nt_ | spCas9 | spCas9 | TTR | TGAGCTAGGCTG | 1038 |
| | 3-12_020 | | NGC IBE | Promoter | CTTATCCCTGC | |
| gRNA-#67 | ABE_NGC_20nt_ | spCas9 | spCas9 | TTR | TGCCAATCTGAC | 1039 |
| | 3-9_008 | | NGC | Promoter | TGCAAACCTGC | |
| | | | ABE | | | |
| gRNA-#67 | CBE_NGC_20nt_ | spCas9 | spCas9 | TTR | TGCCAATCTGAC | 1039 |
| | 4-9_009 | | NGC | Promoter | TGCAAACCTGC | |
| | | | CBE | | | |
| gRNA-#67 | ABE_NGC_20nt_ | spCas9 | spCas9 | TTR | TGCCAATCTGAC | 1039 |
| | 3-12_020 | | NGC IBE | Promoter | TGCAAACCTGC | |
| gRNA-#68 | ABE_NGG_20nt_ | spCas9 | spCas9 | TTR | TGCTCTAATCTC | 1040 |
| | 3-9_002 | | ABE | Promoter | TCTAGACAAGG | |
| gRNA-#68 | CBE_NGG_20nt_ | spCas9 | spCas9 | TTR | TGCTCTAATCTC | 1040 |
| | 4-9_003 | | CBE | Promoter | TCTAGACAAGG | |
| gRNA-#68 | ABE_NGG_20nt_ | spCas9 | spCas9 | TTR | TGCTCTAATCTC | 1040 |
| | 3-12_018 | | IBE | Promoter | TCTAGACAAGG | |
| gRNA-#69 | ABE_NGG_20nt_ | spCas9 | spCas9 | TTR | TGTGATGGCTGC | 1041 |
| | 3-9_002 | | ABE | Promoter | TCCCAGCCTGG | |
| gRNA-#69 | CBE_NGG_20nt_ | spCas9 | spCas9 | TTR | TGTGATGGCTGC | 1041 |
| | 4-9_003 | | CBE | Promoter | TCCCAGCCTGG | |
| gRNA-#69 | ABE_NGG_20nt_ | spCas9 | spCas9 | TTR | TGTGATGGCTGC | 1041 |
| | 3-12_018 | | IBE | Promoter | TCCCAGCCTGG | |
| gRNA-#70 | ABE_NGC_20nt_ | spCas9 | spCas9 | TTR | TTGGCAGGGATA | 1042 |
| | 3-9_008 | | NGC | Promoter | AGCAGCCTAGC | |
| | | | ABE | | | |
| gRNA-#70 | CBE_NGC_20nt_ | spCas9 | spCas9 | TTR | TTGGCAGGGATA | 1042 |
| | 4-9_009 | | NGC | Promoter | AGCAGCCTAGC | |
| | | | CBE | | | |
| gRNA-#70 | ABE_NGC_20nt_ | spCas9 | spCas9 | TTR | TTGGCAGGGATA | 1042 |
| | 3-12_020 | | NGC IBE | Promoter | AGCAGCCTAGC | |
| gRNA-#71 | ABE_NGC_20nt_ | spCas9 | spCas9 | TTR | TTTTATACTCAC | 1043 |
| | 3-9_008 | | NGC | Promoter | TTCTCCTGAGC | |
| | | | ABE | | | |
| gRNA-#71 | CBE_NGC_20nt_ | spCas9 | spCas9 | TTR | TTTTATACTCAC | 1043 |
| | 4-9_009 | | NGC | Promoter | TTCTCCTGAGC | |
| | | | CBE | | | |
| gRNA-#71 | ABE_NGC_20nt_ | spCas9 | spCas9 | TTR | TTTTATACTCAC | 1043 |
| | 3-12_020 | | NGC IBE | Promoter | TTCTCCTGAGC | |

| | | Human | | | | Target |
|---|---|---|---|---|---|---|
| gRNA | | Chromosome | Start | | | Base |
| Name | PAM | Location | Site | End Site | Strand | Position(s) |
| gRNA1594 | GGC | chr18 | 31593011 | 31593034 | − | 8 |
| gRNA1594 | GGC | chr18 | 31593011 | 31593034 | − | 7 |
| gRNA1594 | GGC | chr18 | 31593011 | 31593034 | − | 7 |
| gRNA1595 | AGC | chr18 | 31592994 | 31593017 | − | 9 |
| gRNA1596 | AGC | chr18 | 31592991 | 31593014 | − | 7, 6 |
| gRNA1597 | AGC | chr18 | 31598558 | 31598581 | + | 8 |
| gRNA1597 | AGC | chr18 | 31598558 | 31598581 | + | 8 |
| gRNA1598 | TGG | chr18 | 31595114 | 31595137 | + | 4 |
| gRNA1599 | TGG | chr18 | 31595239 | 31595262 | − | 6 |
| gRNA1600 | TGG | chr18 | 31593012 | 31593035 | − | 8 |
| gRNA1601 | TGC | chr18 | 31595245 | 31595268 | − | 12 |
| gRNA1602 | AGC | chr18 | 31591959 | 31591982 | − | 10 |
| gRNA1603 | TGA | chr18 | 31592883 | 31592906 | + | 11 |
| gRNA1604 | TGA | chr18 | 31595108 | 31595131 | + | 10 |
| gRNA1605 | GGA | chr18 | 31595115 | 31595138 | + | 3 |
| gRNA1606 | GGA | chr18 | 31595238 | 31595261 | − | 5 |
| gRNA1607 | AGA | chr18 | 31591953 | 31591976 | − | 4 |
| gRNA1746 | TGA | chr18 | 31591776 | 31591799 | − | |
| gRNA1746 | TGA | chr18 | 31591776 | 31591799 | − | |
| gRNA1746 | TGA | chr18 | 31591776 | 31591799 | − | |
| gRNA1747 | ACAAAT | chr18 | 31591738 | 31591765 | − | |
| gRNA1747 | ACAAAT | chr18 | 31591738 | 31591765 | − | |
| gRNA1748 | GTGAGT | chr18 | 31591820 | 31591847 | + | |
| gRNA1748 | GTGAGT | chr18 | 31591820 | 31591847 | + | |
| gRNA1749 | GGA | chr18 | 31591880 | 31591903 | + | |
| gRNA1749 | GGA | chr18 | 31591880 | 31591903 | + | |
| gRNA1749 | GGA | chr18 | 31591880 | 31591903 | + | |
| gRNA1750 | AAGAAT | chr18 | 31591890 | 31591917 | − | |
| gRNA1750 | AAGAAT | chr18 | 31591890 | 31591917 | − | |
| gRNA1751 | TGG | chr18 | 31591725 | 31591748 | + | |
| gRNA1751 | TGG | chr18 | 31591725 | 31591748 | + | |
| gRNA1751 | TGG | chr18 | 31591725 | 31591748 | + | |
| gRNA1752 | AGA | chr18 | 31591858 | 31591881 | + | |
| gRNA1752 | AGA | chr18 | 31591858 | 31591881 | + | |
| gRNA1752 | AGA | chr18 | 31591858 | 31591881 | + | |
| gRNA1753 | TGA | chr18 | 31591734 | 31591757 | − | |
| gRNA1753 | TGA | chr18 | 31591734 | 31591757 | − | |
| gRNA1753 | TGA | chr18 | 31591734 | 31591757 | − | |
| gRNA1754 | AGG | chr18 | 31591826 | 31591849 | − | |
| gRNA1754 | AGG | chr18 | 31591826 | 31591849 | − | |
| gRNA1754 | AGG | chr18 | 31591826 | 31591849 | − | |
| gRNA1755 | AGA | chr18 | 31591761 | 31591784 | − | |
| gRNA1755 | AGA | chr18 | 31591761 | 31591784 | − | |
| gRNA1755 | AGA | chr18 | 31591761 | 31591784 | − | |
| gRNA1756 | AGA | chr18 | 31591720 | 31591743 | − | |
| gRNA1756 | AGA | chr18 | 31591720 | 31591743 | − | |
| gRNA1756 | AGA | chr18 | 31591720 | 31591743 | − | |
| gRNA1757 | CAGGAT | chr18 | 31591877 | 31591904 | + | |
| gRNA1757 | CAGGAT | chr18 | 31591877 | 31591904 | + | |
| gRNA1758 | AGAAGT | chr18 | 31591857 | 31591884 | + | |
| gRNA1758 | AGAAGT | chr18 | 31591857 | 31591884 | + | |
| gRNA1759 | GGA | chr18 | 31591883 | 31591906 | − | |
| gRNA1759 | GGA | chr18 | 31591883 | 31591906 | − | |
| gRNA1759 | GGA | chr18 | 31591883 | 31591906 | − | |
| gRNA1760 | CTTAGT | chr18 | 31591770 | 31591797 | − | |
| gRNA1760 | CTTAGT | chr18 | 31591770 | 31591797 | − | |
| gRNA1761 | CAAGGT | chr18 | 31591707 | 31591734 | + | |
| gRNA1761 | CAAGGT | chr18 | 31591707 | 31591734 | + | |
| gRNA1762 | GCAGGT | chr18 | 31591771 | 31591798 | + | |
| gRNA1762 | GCAGGT | chr18 | 31591771 | 31591798 | + | |
| gRNA1763 | ATGGGT | chr18 | 31591723 | 31591750 | + | |
| gRNA1763 | ATGGGT | chr18 | 31591723 | 31591750 | + | |
| gRNA1764 | AGA | chr18 | 31591713 | 31591736 | − | |
| gRNA1764 | AGA | chr18 | 31591713 | 31591736 | − | |
| gRNA1764 | AGA | chr18 | 31591713 | 31591736 | − | |
| gRNA1765 | AGG | chr18 | 31591879 | 31591902 | + | |
| gRNA1765 | AGG | chr18 | 31591879 | 31591902 | + | |
| gRNA1765 | AGG | chr18 | 31591879 | 31591902 | + | |
| gRNA1766 | AGA | chr18 | 31591786 | 31591809 | + | |
| gRNA1766 | AGA | chr18 | 31591786 | 31591809 | + | |
| gRNA1766 | AGA | chr18 | 31591786 | 31591809 | + | |
| gRNA1767 | TGG | chr18 | 31591871 | 31591894 | − | |
| gRNA1767 | TGG | chr18 | 31591871 | 31591894 | − | |
| gRNA1767 | TGG | chr18 | 31591871 | 31591894 | − | |
| gRNA1768 | TGA | chr18 | 31591783 | 31591806 | − | |
| gRNA1768 | TGA | chr18 | 31591783 | 31591806 | − | |
| gRNA1768 | TGA | chr18 | 31591783 | 31591806 | − | |
| gRNA1769 | ATAAGT | chr18 | 31591751 | 31591778 | − | |
| gRNA1769 | ATAAGT | chr18 | 31591751 | 31591778 | − | |
| gRNA1770 | AGA | chr18 | 31591817 | 31591840 | + | |
| gRNA1770 | AGA | chr18 | 31591817 | 31591840 | + | |
| gRNA1770 | AGA | chr18 | 31591817 | 31591840 | + | |
| gRNA1771 | AGA | chr18 | 31591892 | 31591915 | − | |
| gRNA1771 | AGA | chr18 | 31591892 | 31591915 | − | |
| gRNA1771 | AGA | chr18 | 31591892 | 31591915 | − | |
| gRNA1772 | TGG | chr18 | 31591884 | 31591907 | − | |
| gRNA1772 | TGG | chr18 | 31591884 | 31591907 | − | |
| gRNA1772 | TGG | chr18 | 31591884 | 31591907 | − | |
| gRNA1773 | TGA | chr18 | 31591832 | 31591855 | − | |
| gRNA1773 | TGA | chr18 | 31591832 | 31591855 | − | |
| gRNA1773 | TGA | chr18 | 31591832 | 31591855 | − | |
| gRNA1774 | AGAAGT | chr18 | 31591816 | 31591843 | + | |
| gRNA1774 | AGAAGT | chr18 | 31591816 | 31591843 | + | |
| gRNA1775 | CGG | chr18 | 31591706 | 31591729 | − | |
| gRNA1775 | CGG | chr18 | 31591706 | 31591729 | − | |
| gRNA1775 | CGG | chr18 | 31591706 | 31591729 | − | |
| gRNA1776 | GGG | chr18 | 31591855 | 31591878 | − | |
| gRNA1776 | GGG | chr18 | 31591855 | 31591878 | − | |
| gRNA1776 | GGG | chr18 | 31591855 | 31591878 | − | |
| gRNA1777 | CTAAGT | chr18 | 31591750 | 31591777 | + | |
| gRNA1777 | CTAAGT | chr18 | 31591750 | 31591777 | + | |
| gRNA1778 | GTCAAT | chr18 | 31591754 | 31591781 | + | |
| gRNA1778 | GTCAAT | chr18 | 31591754 | 31591781 | + | |
| gRNA1779 | AGA | chr18 | 31591759 | 31591782 | − | |
| gRNA1779 | AGA | chr18 | 31591759 | 31591782 | − | |
| gRNA1779 | AGA | chr18 | 31591759 | 31591782 | − | |
| gRNA1780 | GAGAAT | chr18 | 31591755 | 31591782 | − | |
| gRNA1780 | GAGAAT | chr18 | 31591755 | 31591782 | − | |
| gRNA1781 | TCAGAT | chr18 | 31591783 | 31591810 | + | |
| gRNA1781 | TCAGAT | chr18 | 31591783 | 31591810 | + | |
| gRNA1782 | TGG | chr18 | 31591883 | 31591906 | + | |
| gRNA1782 | TGG | chr18 | 31591883 | 31591906 | + | |
| gRNA1782 | TGG | chr18 | 31591883 | 31591906 | + | |
| gRNA1783 | AATAAT | chr18 | 31591757 | 31591784 | + | |
| gRNA1783 | AATAAT | chr18 | 31591757 | 31591784 | + | |
| gRNA1784 | ATGAGT | chr18 | 31591886 | 31591913 | − | |
| gRNA1784 | ATGAGT | chr18 | 31591886 | 31591913 | − | |
| gRNA1785 | GCCAAT | chr18 | 31591808 | 31591835 | − | |
| gRNA1785 | GCCAAT | chr18 | 31591808 | 31591835 | − | |
| gRNA1786 | TGG | chr18 | 31591842 | 31591865 | + | |
| gRNA1786 | TGG | chr18 | 31591842 | 31591865 | + | |
| gRNA1786 | TGG | chr18 | 31591842 | 31591865 | + | |
| gRNA1787 | GGG | chr18 | 31591854 | 31591877 | − | |
| gRNA1787 | GGG | chr18 | 31591854 | 31591877 | − | |
| gRNA1787 | GGG | chr18 | 31591854 | 31591877 | − | |
| gRNA1788 | TGTGAT | chr18 | 31591873 | 31591900 | − | |
| gRNA1788 | TGTGAT | chr18 | 31591873 | 31591900 | − | |
| gRNA1789 | GCTGAT | chr18 | 31591788 | 31591815 | − | |
| gRNA1789 | GCTGAT | chr18 | 31591788 | 31591815 | − | |
| gRNA1790 | AGA | chr18 | 31591765 | 31591788 | + | |
| gRNA1790 | AGA | chr18 | 31591765 | 31591788 | + | |
| gRNA1790 | AGA | chr18 | 31591765 | 31591788 | + | |
| gRNA1791 | AGA | chr18 | 31591757 | 31591780 | − | |
| gRNA1791 | AGA | chr18 | 31591757 | 31591780 | − | |
| gRNA1791 | AGA | chr18 | 31591757 | 31591780 | − | |
| gRNA1792 | CAGAAT | chr18 | 31591763 | 31591790 | + | |
| gRNA1792 | CAGAAT | chr18 | 31591763 | 31591790 | 1 | |
| gRNA-#1 | AGC | chr18 | 31591849 | 31591872 | + | |
| gRNA-#1 | AGC | chr18 | 31591849 | 31591872 | + | |
| gRNA-#1 | AGC | chr18 | 31591849 | 31591872 | + | |
| gRNA-#2 | GGC | chr18 | 31591839 | 31591862 | + | |
| gRNA-#2 | GGC | chr18 | 31591839 | 31591862 | + | |
| gRNA-#2 | GGC | chr18 | 31591839 | 31591862 | + | |
| gRNA-#3 | TGCAGT | chr18 | 31591778 | 31591805 | + | |
| gRNA-#3 | TGCAGT | chr18 | 31591778 | 31591805 | + | |
| gRNA-#4 | AGA | chr18 | 31591718 | 31591741 | − | |
| gRNA-#4 | AGA | chr18 | 31591718 | 31591741 | − | |
| gRNA-#4 | AGA | chr18 | 31591718 | 31591741 | − | |
| gRNA-#5 | GGC | chr18 | 31591870 | 31591893 | − | |
| gRNA-#5 | GGC | chr18 | 31591870 | 31591893 | − | |
| gRNA-#5 | GGC | chr18 | 31591870 | 31591893 | − | |
| gRNA-#6 | AGAGAT | chr18 | 31591717 | 31591744 | − | |
| gRNA-#6 | AGAGAT | chr18 | 31591717 | 31591744 | − | |
| gRNA-#7 | GGC | chr18 | 31591876 | 31591899 | + | |
| gRNA-#7 | GGC | chr18 | 31591876 | 31591899 | + | |
| gRNA-#7 | GGC | chr18 | 31591876 | 31591899 | + | |
| gRNA-#8 | AGC | chr18 | 31591711 | 31591734 | − | |
| gRNA-#8 | AGC | chr18 | 31591711 | 31591734 | − | |
| gRNA-#8 | AGC | chr18 | 31591711 | 31591734 | − | |
| gRNA-#9 | TGA | chr18 | 31591888 | 31591911 | − | |
| gRNA-#9 | TGA | chr18 | 31591888 | 31591911 | − | |
| gRNA-#9 | TGA | chr18 | 31591888 | 31591911 | − | |
| gRNA-#10 | GGC | chr18 | 31591791 | 31591814 | + | |
| gRNA-#10 | GGC | chr18 | 31591791 | 31591814 | + | |
| gRNA-#10 | GGC | chr18 | 31591791 | 31591814 | + | |
| gRNA-#11 | AGG | chr18 | 31591814 | 31591837 | + | |
| gRNA-#11 | AGG | chr18 | 31591814 | 31591837 | 1 | |
| gRNA-#11 | AGG | chr18 | 31591814 | 31591837 | + | |
| gRNA-#12 | AGG | chr18 | 31591794 | 31591817 | + | |
| gRNA-#12 | AGG | chr18 | 31591794 | 31591817 | + | |
| gRNA-#12 | AGG | chr18 | 31591794 | 31591817 | + | |
| gRNA-#13 | GGA | chr18 | 31591844 | 31591867 | + | |
| gRNA-#13 | GGA | chr18 | 31591844 | 31591867 | + | |
| gRNA-#13 | GGA | chr18 | 31591844 | 31591867 | + | |
| gRNA-#14 | AGG | chr18 | 31591774 | 31591797 | + | |
| gRNA-#14 | AGG | chr18 | 31591774 | 31591797 | + | |
| gRNA-#14 | AGG | chr18 | 31591774 | 31591797 | + | |
| gRNA-#15 | TGC | chr18 | 31591779 | 31591802 | + | |
| gRNA-#15 | TGC | chr18 | 31591779 | 31591802 | + | |
| gRNA-#15 | TGC | chr18 | 31591779 | 31591802 | + | |
| gRNA-#16 | ATTA | chr18 | 31591758 | 31591784 | − | |
| gRNA-#17 | ATTO | chr18 | 31591755 | 31591781 | + | |
| gRNA-#18 | ATTC | chr18 | 31591764 | 31591790 | − | |
| gRNA-#19 | ATTC | chr18 | 31591890 | 31591916 | + | |
| gRNA-#20 | ATTG | chr18 | 31591755 | 31591781 | − | |
| gRNA-#21 | ATTG | chr18 | 31591808 | 31591834 | + | |
| gRNA-#22 | ATTT | chr18 | 31591738 | 31591764 | + | |
| gRNA-#23 | TGA | chr18 | 31591874 | 31591897 | − | |
| gRNA-#23 | TGA | chr18 | 31591874 | 31591897 | − | |
| gRNA-#23 | TGA | chr18 | 31591874 | 31591897 | − | |
| gRNA-#24 | TGA | chr18 | 31591789 | 31591812 | − | |
| gRNA-#24 | TGA | chr18 | 31591789 | 31591812 | − | |
| gRNA-#24 | TGA | chr18 | 31591789 | 31591812 | − | |
| gRNA-#25 | TGG | chr18 | 31591875 | 31591898 | + | |
| gRNA-#25 | TGG | chr18 | 31591875 | 31591898 | + | |
| gRNA-#25 | TGG | chr18 | 31591875 | 31591898 | + | |
| gRNA-#26 | TGC | chr18 | 31591897 | 31591920 | − | |
| gRNA-#26 | TGC | chr18 | 31591897 | 31591920 | − | |
| gRNA-#26 | TGC | chr18 | 31591897 | 31591920 | − | |
| gRNA-#27 | TGG | chr18 | 31591790 | 31591813 | + | |
| gRNA-#27 | TGG | chr18 | 31591790 | 31591813 | + | |
| gRNA-#27 | TGG | chr18 | 31591790 | 31591813 | + | |
| gRNA-#28 | TGC | chr18 | 31591898 | 31591921 | + | |
| gRNA-#28 | TGC | chr18 | 31591898 | 31591921 | + | |
| gRNA-#28 | TGC | chr18 | 31591898 | 31591921 | + | |
| gRNA-#29 | AGGGAT | chr18 | 31591793 | 31591820 | + | |
| gRNA-#29 | AGGGAT | chr18 | 31591793 | 31591820 | + | |
| gRNA-#30 | AGC | chr18 | 31591801 | 31591824 | + | |
| gRNA-#30 | AGC | chr18 | 31591801 | 31591824 | + | |
| gRNA-#30 | AGC | chr18 | 31591801 | 31591824 | + | |
| gRNA-#31 | GGC | chr18 | 31591884 | 31591907 | + | |
| gRNA-#31 | GGC | chr18 | 31591884 | 31591907 | + | |
| gRNA-#31 | GGC | chr18 | 31591884 | 31591907 | + | |
| gRNA-#32 | AGC | chr18 | 31591771 | 31591794 | + | |
| gRNA-#32 | AGC | chr18 | 31591771 | 31591794 | + | |
| gRNA-#32 | AGC | chr18 | 31591771 | 31591794 | + | |
| gRNA-#33 | CTTA | chr18 | 31591750 | 31591776 | − | |
| gRNA-#34 | TGC | chr18 | 31591800 | 31591823 | − | |
| gRNA-#34 | TGC | chr18 | 31591800 | 31591823 | − | |
| gRNA-#34 | TGC | chr18 | 31591800 | 31591823 | − | |
| gRNA-#35 | CTTA | chr18 | 31591797 | 31591823 | − | |
| gRNA-#36 | CTTA | chr18 | 31591752 | 31591778 | + | |
| gRNA-#37 | CTTC | chr18 | 31591816 | 31591842 | − | |
| gRNA-#38 | AGC | chr18 | 31591860 | 31591883 | − | |
| gRNA-#38 | AGC | chr18 | 31591860 | 31591883 | − | |
| gRNA-#38 | AGC | chr18 | 31591860 | 31591883 | − | |
| gRNA-#39 | CTTC | chr18 | 31591857 | 31591883 | − | |
| gRNA-#40 | CTTG | chr18 | 31591893 | 31591919 | + | |
| gRNA-#41 | CTTG | chr18 | 31591706 | 31591732 | − | |
| gRNA-#42 | CTTT | chr18 | 31591762 | 31591788 | + | |
| gRNA-#43 | CTTT | chr18 | 31591828 | 31591854 | − | |
| gRNA-#44 | AGG | chr18 | 31591838 | 31591861 | + | |
| gRNA-#44 | AGG | chr18 | 31591838 | 31591861 | + | |
| gRNA-#44 | AGG | chr18 | 31591838 | 31591861 | + | |
| gRNA-#45 | GGG | chr18 | 31591726 | 31591749 | + | |
| gRNA-#45 | GGG | chr18 | 31591726 | 31591749 | + | |
| gRNA-#45 | GGG | chr18 | 31591726 | 31591749 | + | |
| gRNA-#46 | GGG | chr18 | 31591843 | 31591866 | + | |
| gRNA-#46 | GGG | chr18 | 31591843 | 31591866 | + | |
| gRNA-#46 | GGG | chr18 | 31591843 | 31591866 | + | |
| gRNA-#47 | TGC | chr18 | 31591867 | 31591890 | − | |
| gRNA-#47 | TGC | chr18 | 31591867 | 31591890 | − | |
| gRNA-#47 | TGC | chr18 | 31591867 | 31591890 | − | |
| gRNA-#48 | GGC | chr18 | 31591853 | 31591876 | − | |
| gRNA-#48 | GGC | chr18 | 31591853 | 31591876 | − | |
| gRNA-#48 | GGC | chr18 | 31591853 | 31591876 | − | |
| gRNA-#48 | TGA | chr18 | 31591822 | 31591845 | + | |
| gRNA-#48 | TGA | chr18 | 31591822 | 31591845 | + | |
| gRNA-#48 | TGA | chr18 | 31591822 | 31591845 | + | |
| gRNA-#50 | TGA | chr18 | 31591804 | 31591827 | − | |
| gRNA-#50 | TGA | chr18 | 31591804 | 31591827 | − | |
| gRNA-#50 | TGA | chr18 | 31591804 | 31591827 | − | |
| gRNA-#51 | GGA | chr18 | 31591815 | 31591838 | + | |
| gRNA-#51 | GGA | chr18 | 31591815 | 31591838 | + | |
| gRNA-#51 | GGA | chr18 | 31591815 | 31591838 | + | |
| gRNA-#52 | GGG | chr18 | 31591795 | 31591818 | + | |
| gRNA-#52 | GGG | chr18 | 31591795 | 31591818 | + | |
| gRNA-#52 | GGG | chr18 | 31591795 | 31591818 | + | |
| gRNA-#53 | TGA | chr18 | 31591748 | 31591771 | + | |
| gRNA-#53 | TGA | chr18 | 31591748 | 31591771 | + | |
| gRNA-#53 | TGA | chr18 | 31591748 | 31591771 | + | |
| gRNA-#54 | GTTA | chr18 | 31591748 | 31591774 | + | |
| gRNA-#55 | GTTC | chr18 | 31591732 | 31591758 | + | |
| gRNA-#56 | GTTG | chr18 | 31591766 | 31591792 | + | |
| gRNA-#57 | GGA | chr18 | 31591796 | 31591819 | + | |
| gRNA-#57 | GGA | chr18 | 31591796 | 31591819 | + | |
| gRNA-#57 | GGA | chr18 | 31591796 | 31591819 | + | |
| gRNA-#58 | GTTT | chr18 | 31591796 | 31591822 | + | |
| gRNA-#59 | AGA | chr18 | 31591722 | 31591745 | − | |
| gRNA-#59 | AGA | chr18 | 31591722 | 31591745 | − | |
| gRNA-#59 | AGA | chr18 | 31591722 | 31591745 | − | |
| gRNA-#60 | GGC | chr18 | 31591825 | 31591848 | − | |
| gRNA-#60 | GGC | chr18 | 31591825 | 31591848 | − | |
| gRNA-#60 | GGC | chr18 | 31591825 | 31591848 | − | |
| gRNA-#61 | AGC | chr18 | 31591846 | 31591869 | + | |
| gRNA-#61 | AGC | chr18 | 31591846 | 31591869 | + | |
| gRNA-#61 | AGC | chr18 | 31591846 | 31591869 | + | |
| gRNA-#62 | TGC | chr18 | 31591822 | 31591845 | − | |
| gRNA-#62 | TGC | chr18 | 31591822 | 31591845 | − | |
| gRNA-#62 | TGC | chr18 | 31591822 | 31591845 | − | |
| gRNA-#63 | AGC | chr18 | 31591804 | 31591827 | + | |
| gRNA-#63 | AGC | chr18 | 31591804 | 31591827 | + | |
| gRNA-#63 | AGC | chr18 | 31591804 | 31591827 | + | |
| gRNA-#64 | AGC | chr18 | 31591832 | 31591855 | + | |
| gRNA-#64 | AGC | chr18 | 31591832 | 31591855 | + | |
| gRNA-#64 | AGC | chr18 | 31591832 | 31591855 | + | |
| gRNA-#65 | TCTGAT | chr18 | 31591782 | 31591809 | − | |
| gRNA-#65 | TCTGAT | chr18 | 31591782 | 31591809 | − | |
| gRNA-#66 | TGC | chr18 | 31591812 | 31591835 | − | |
| gRNA-#66 | TGC | chr18 | 31591812 | 31591835 | − | |
| gRNA-#66 | TGC | chr18 | 31591812 | 31591835 | − | |
| gRNA-#67 | TGC | chr18 | 31591792 | 31591815 | − | |
| gRNA-#67 | TGC | chr18 | 31591792 | 31591815 | − | |
| gRNA-#67 | TGC | chr18 | 31591792 | 31591815 | − | |
| gRNA-#68 | AGG | chr18 | 31591710 | 31591733 | + | |
| gRNA-#68 | AGG | chr18 | 31591710 | 31591733 | + | |
| gRNA-#68 | AGG | chr18 | 31591710 | 31591733 | + | |
| gRNA-#69 | TGG | chr18 | 31591856 | 31591879 | − | |
| gRNA-#69 | TGG | chr18 | 31591856 | 31591879 | − | |
| gRNA-#69 | TGG | chr18 | 31591856 | 31591879 | − | |
| gRNA-#70 | AGC | chr18 | 31591809 | 31591832 | + | |
| gRNA-#70 | AGC | chr18 | 31591809 | 31591832 | + | |
| gRNA-#70 | AGC | chr18 | 31591809 | 31591832 | + | |
| gRNA-#71 | AGC | chr18 | 31591830 | 31591853 | − | |
| gRNA-#71 | AGC | chr18 | 31591830 | 31591853 | − | |
| gRNA-#71 | AGC | chr18 | 31591830 | 31591853 | − | |

The spacer sequences in Table 2A corresponding to sgRNAs sgRNA_361, sgRNA_362, sgRNA_363, sgRNA_364, sgRNA_365, sgRNA_366, and sgRNA_367 can be used for targeting a base editor to alter a nucleobase of a splice site of the transthyretin polynucleotide. The spacer sequences in Table 2A corresponding to sgRNAs sgRNA_368, sgRNA_369, sgRNA_370, sgRNA_371, sgRNA_372, sgRNA_373, and sgRNA_374 can be used for targeting an endonuclease to a transthyretin (TTR) polynucleotide sequence. The three spacer sequences in Table 2A corresponding to sgRNA_375, sgRNA_376, and sgRNA_377 can be used to alter a nucleobase of a transthyretin (TTR) polynucleotide. The alteration of the nucleobase can result in an alteration of an isoleucine (I) to a valine (V) (e.g., to correct a V122I mutation in a transthyretin polypeptide encoded by the transthyretin polynucleotide). In embodiments, a transthyretin polynucleotide can be edited using the following combinations of base editors and sgRNA sequences (see Tables 1 and 2A): ABE8.8 and sgRNA_361; ABE8.8 and sgRNA_362; ABE8.8-VRQR and sgRNA_363; BE4-VRQR and sgRNA_363; BE4-VRQR and sgRNA_364; saABE8.8 and sgRNA_365; saBE4 and sgRNA_365; saBE4-KKH and sgRNA_366, ABE-bhCas12b and sgRNA_367; spCas9-ABE and sgRNA_375; spCas9-VRQR-ABE and sgRNA_376; or saCas9-ABE and sgRNA_377. The PAM sequence of spCas9-ABE can be AGG. The PAM sequence of spCas9-VRQR-ABE can be GGA. The PAM sequence of saCas9-ABE can be AGGAAT.

In certain embodiments, the fusion proteins provided herein comprise one or more features that improve the base editing activity of the fusion proteins. For example, any of the fusion proteins provided herein may comprise a Cas9 domain that has reduced nuclease activity. In some embodiments, any of the fusion proteins provided herein may have a Cas9 domain that does not have nuclease activity (dCas9), or a Cas9 domain that cuts one strand of a duplexed DNA molecule, referred to as a Cas9 nickase (nCas9). Without wishing to be bound by any particular theory, the presence of the catalytic residue (e.g., H₈₄₀) maintains the activity of the Cas9 to cleave the non-edited (e.g., non-methylated) strand opposite the targeted nucleobase. Mutation of the catalytic residue (e.g., D10 to A¹⁰) prevents cleavage of the edited strand containing the targeted A residue. Such Cas9 variants can generate a single-strand DNA break (nick) at a specific location based on the gRNA-defined target sequence, leading to repair of the non-edited strand, ultimately resulting in a nucleobase change on the non-edited strand.

### Nucleobase Editors

Useful in the methods and compositions described herein are nucleobase editors that edit, modify or alter a target nucleotide sequence of a polynucleotide. Nucleobase editors described herein typically include a polynucleotide programmable nucleotide binding domain and a nucleobase editing domain (e.g., adenosine deaminase, cytidine deaminase, or a dual deaminase). A polynucleotide programmable nucleotide binding domain, when in conjunction with a bound guide polynucleotide (e.g., gRNA), can specifically bind to a target polynucleotide sequence and thereby localize the base editor to the target nucleic acid sequence desired to be edited.

### Polynucleotide Programmable Nucleotide Binding Domain

Polynucleotide programmable nucleotide binding domains bind polynucleotides (e.g., RNA, DNA). A polynucleotide programmable nucleotide binding domain of a base editor can itself comprise one or more domains (e.g., one or more nuclease domains). In some embodiments, the nuclease domain of a polynucleotide programmable nucleotide binding domain comprises an endonuclease or an exonuclease.

Disclosed herein are base editors comprising a polynucleotide programmable nucleotide binding domain comprising all or a portion (e.g., a functional portion) of a CRISPR protein (i.e., a base editor comprising as a domain all or a portion (e.g., a functional portion) of a CRISPR protein (e.g., a Cas protein), also referred to as a “CRISPR protein-derived domain” of the base editor). A CRISPR protein-derived domain incorporated into a base editor can be modified compared to a wild-type or natural version of the CRISPR protein. A CRISPR protein-derived domain can comprise one or more mutations, insertions, deletions, rearrangements and/or recombinations relative to a wild-type or natural version of the CRISPR protein.

Cas proteins that can be used herein include class 1 and class 2. Non-limiting examples of Cas proteins include Cas1, Cas1B, Cas2, Cas3, Cas4, Cas5, Cas5d, Cas5t, Cas5h, Cas5a, Cas6, Cas7, Cas8, Cas9 (also known as Csn1 or Csx12), Cas10, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Csd1, Csd2, Cst1, Cst2, Csh1, Csh2, Csa1, Csa2, Csa3, Csa4, Csa5, Cas12a/Cpf1, Cas12b/C₂cl (e.g., SEQ ID NO: 232), Cas12c/C₂c3, Cas12d/CasY, Cas12e/CasX, Cas12g, Cas12h, Cas12i, and Cas12j/CasΘ, CARF, DinG, homologues thereof, or modified versions thereof. A CRISPR enzyme can direct cleavage of one or both strands at a target sequence, such as within a target sequence and/or within a complement of a target sequence. For example, a CRISPR enzyme can direct cleavage of one or both strands within about 1, 2, 3, 4, 5, 6, 7, 8, 9, 10, 15, 20, 25, 50, 100, 200, 500, or more base pairs from the first or last nucleotide of a target sequence.

A vector that encodes a CRISPR enzyme that is mutated to with respect to a corresponding wild-type enzyme such that the mutated CRISPR enzyme lacks the ability to cleave one or both strands of a target polynucleotide containing a target sequence can be used. A Cas protein (e.g., Cas9, Cas12) or a Cas domain (e.g., Cas9, Cas12) can refer to a polypeptide or domain with at least or at least about 50%, 60%, 70%, 80%, 90%, 91%, 92%, 93%, 94%, 95%, 96%, 97%, 98%, 99%, or 100% sequence identity and/or sequence homology to a wild-type exemplary Cas polypeptide or Cas domain. Cas (e.g., Cas9, Cas12) can refer to the wild-type or a modified form of the Cas protein that can comprise an amino acid change such as a deletion, insertion, substitution, variant, mutation, fusion, chimera, or any combination thereof.

In some embodiments, a CRISPR protein-derived domain of a base editor can include all or a portion (e.g., a functional portion) of Cas9 from Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquis (NCBI Ref: NC_018721.1); Streptococcus thermophilus (NCBI Ref: YP_820832.1); Listeria innocua (NCBI Ref: NP_472073.1); Campylobacter jejuni (NCBI Ref: YP_002344900.1); Neisseria meningitidis (NCBI Ref: YP_002342100.1), Streptococcus pyogenes, or Staphylococcus aureus.

Some aspects of the disclosure provide high fidelity Cas9 domains. High fidelity Cas9 domains are known in the art and described, for example, in Kleinstiver, B. P., et al. “High-fidelity CRISPR-Cas9 nucleases with no detectable genome-wide off-target effects.” Nature 529, 490-495 (2016); and Slaymaker, I. M., et al. “Rationally engineered Cas9 nucleases with improved specificity.” Science 351, 84-88 (2015); the entire contents of each of which are incorporated herein by reference. An Exemplary high fidelity Cas9 domain is provided in the Sequence Listing as SEQ ID NO: 233.

In some embodiments, any of the Cas9 fusion proteins or complexes provided herein comprise one or more of a D10A, N497X, a R661X, a Q695X, and/or a Q926X mutation, or a corresponding mutation in any of the amino acid sequences provided herein, wherein X is any amino acid.

Typically, Cas9 proteins, such as Cas9 from S. pyogenes (spCas9), require a “protospacer adjacent motif (PAM)” or PAM-like motif, which is a 2-6 base pair DNA sequence immediately following the DNA sequence targeted by the Cas9 nuclease in the CRISPR bacterial adaptive immune system. The presence of an NGG PAM sequence is required to bind a particular nucleic acid region, where the “N” in “NGG” is adenosine (A), thymidine (T), or cytosine (C), and the G is guanosine. In some embodiments, any of the fusion proteins or complexes provided herein may contain a Cas9 domain that is capable of binding a nucleotide sequence that does not contain a canonical (e.g., NGG) PAM sequence. Cas9 domains that bind to non-canonical PAM sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); the entire contents of each are hereby incorporated by reference.

In some embodiments, the napDNAbp is a circular permutant (e.g., SEQ ID NO: 238).

In some embodiments, the polynucleotide programmable nucleotide binding domain comprises a nickase domain. Herein the term “nickase” refers to a polynucleotide programmable nucleotide binding domain comprising a nuclease domain that is capable of cleaving only one strand of the two strands in a duplexed nucleic acid molecule (e.g., DNA). For example, where a polynucleotide programmable nucleotide binding domain comprises a nickase domain derived from Cas9, the Cas9-derived nickase domain can include a D10A mutation and a histidine at position 840. In another example, a Cas9-derived nickase domain comprises an H840A mutation, while the amino acid residue at position 10 remains a D.

In some embodiments, a Cas9 nuclease has an inactive (e.g., an inactivated) DNA cleavage domain, that is, the Cas9 is a nickase, referred to as an “nCas9” protein (for “nickase” Cas9; SEQ ID NO: 201). The Cas9 nickase may be a Cas9 protein that is capable of cleaving only one strand of a duplexed nucleic acid molecule (e.g., a duplexed DNA molecule). In some embodiments the Cas9 nickase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the Cas9 nickases provided herein. Additional suitable Cas9 nickases will be apparent to those of skill in the art based on this disclosure and knowledge in the field and are within the scope of this disclosure.

Also provided herein are base editors comprising a polynucleotide programmable nucleotide binding domain which is catalytically dead (i.e., incapable of cleaving a target polynucleotide sequence). For example, in the case of a base editor comprising a Cas9 domain, the Cas9 can comprise both a D10A mutation and an H840A mutation. In further embodiments, a catalytically dead polynucleotide programmable nucleotide binding domain comprises a point mutation (e.g., D10A or H840A) as well as a deletion of all or a portion (e.g., a functional portion) of a nuclease domain. dCas9 domains are known in the art and described, for example, in Qi et al., “Repurposing CRISPR as an RNA-guided platform for sequence-specific control of gene expression.” Cell. 2013; 152 (5): 1173-83, the entire contents of which are incorporated herein by reference.

The term “protospacer adjacent motif (PAM)” or PAM-like motif refers to a 2-6 base pair DNA sequence immediately following the DNA sequence targeted by a nucleic acid programmable DNA binding protein. In some embodiments, the PAM can be a 5′ PAM (i.e., located upstream of the 5′ end of the protospacer). In other embodiments, the PAM can be a 3′ PAM (i.e., located downstream of the 5′ end of the protospacer). The PAM sequence can be any PAM sequence known in the art. Suitable PAM sequences include, but are not limited to, NGG, NGA, NGC, NGN, NGT, NGTT, NGCG, NGAG, NGAN, NGNG, NGCN, NGCG, NGTN, NNGRRT, NNNRRT, NNGRR (N), TTTV, TYCV, TYCV, TATV, NNNNGATT, NNAGAAW, or NAAAAC. Y is a pyrimidine; N is any nucleotide base; W is A or T.

A base editor provided herein can comprise a CRISPR protein-derived domain that is capable of binding a nucleotide sequence that contains a canonical or non-canonical protospacer adjacent motif (PAM) sequence.

In some embodiments, the PAM is an “NRN” PAM where the “N” in “NRN” is adenine (A), thymine (T), guanine (G), or cytosine (C), and the R is adenine (A) or guanine (G); or the PAM is an “NYN” PAM, wherein the “N” in NYN is adenine (A), thymine (T), guanine (G), or cytosine (C), and the Y is cytidine (C) or thymine (T), for example, as described in R.T. Walton et al., 2020, Science, 10.1126/science.aba8853 (2020), the entire contents of which are incorporated herein by reference.

Several PAM variants are described in Table 3 below.

TABLE 3
Cas9 proteins and corresponding PAM sequences.
N is A, C, T, or G; and Vis A, C, or G.

| Variant | PAM |
|---|---|
| spCas9 | NGG |
| spCas9-VRQR | NGA |
| spCas9-VRER | NGCG |
| xCas9 (sp) | NGN |
| saCas9 | NNGRRT |
| saCas9-KKH | NNNRRT |
| spCas9-MQKSER | NGCG |
| spCas9-MQKSER | NGCN |
| spCas9-LRKIQK | NGTN |
| spCas9-LRVSQK | NGTN |
| spCas9-LRVSQL | NGTN |
| spCas9-MQKFRAER | NGC |
| Cpf1 | 5′ (TTTV) |
| SpyMac | 5′-NAA-3′ |

In some embodiments, the PAM is NGC. In some embodiments, the NGC PAM is recognized by a Cas9 variant. In some embodiments, the NGC PAM Cas9 variant includes one or more amino acid substitutions selected from D1135M, S1136Q, G1218K, E1219F, A1322R, D1332A, R1335E, and T1337R (collectively termed “MQKFRAER”) of spCas9 (SEQ ID No: 197), or a corresponding mutation in another Cas9. In some embodiments, the Cas9 variant contains one or more amino acid substitutions selected from D1135V, G1218R, R1335Q, and T1337R (collectively termed VRQR) of spCas9 (SEQ ID No: 197), or a corresponding mutation in another Cas9. In some embodiments, the Cas9 variant contains one or more amino acid substitutions selected from D1135V, G1218R, R1335E, and T1337R (collectively termed VRER) of spCas9 (SEQ ID No: 197), or a corresponding mutation in another Cas9. In some embodiments, the Cas9 variant contains one or more amino acid substitutions selected from E782K, N968K, and R1015H (collectively termed KHH) of saCas9 (SEQ ID NO: 218). In some embodiments, the Cas9 variant includes one or more amino acid substitutions selected from D1135M, S1136Q, G1218K, E1219S, R1335E, and T1337R (collectively termed “MQKSER”) of spCas9 (SEQ ID No: 197), or a corresponding mutation in another Cas9. In some embodiments, the Cas9 variant includes one or more amino acid substitutions selected from D1135M, S1136Q, G1218K, E1219S, R1335E, and T1337R (collectively termed “MQKSER”) of spCas9 (SEQ ID No: 197), or a corresponding mutation in another Cas9.

In some embodiments, a CRISPR protein-derived domain of a base editor comprises all or a portion (e.g., a functional portion) of a Cas9 protein with a canonical PAM sequence (NGG). In other embodiments, a Cas9-derived domain of a base editor can employ a non-canonical PAM sequence. Such sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); R. T. Walton et al. “Unconstrained genome targeting with near-PAMless engineered CRISPR-Cas9 variants” Science 10.1126/science.aba8853 (2020); Hu et al. “Evolved Cas9 variants with broad PAM compatibility and high DNA specificity,” Nature, 2018 Apr. 5, 556 (7699), 57-63; Miller et al., “Continuous evolution of SpCas9 variants compatible with non-G PAMs” Nat. Biotechnol., 2020 April;38 (4): 471-481; the entire contents of each are hereby incorporated by reference.

Fusion Proteins or Complexes Comprising a NapDNAbp and a Cytidine Deaminase and/or Adenosine Deaminase

Some aspects of the disclosure provide fusion proteins or complexes comprising a Cas9 domain or other nucleic acid programmable DNA binding protein (e.g., Cas12) and one or more cytidine deaminase, adenosine deaminase, or cytidine adenosine deaminase domains. It should be appreciated that the Cas9 domain may be any of the Cas9 domains or Cas9 proteins (e.g., dCas9 or nCas9) provided herein. In some embodiments, any of the Cas9 domains or Cas9 proteins (e.g., dCas9 or nCas9) provided herein may be fused with any of the cytidine deaminases and/or adenosine deaminases provided herein. The domains of the base editors disclosed herein can be arranged in any order.

In some embodiments, the fusion proteins or complexes comprising a cytidine deaminase or adenosine deaminase and a napDNAbp (e.g., Cas9 or Cas12 domain) do not include a linker sequence. In some embodiments, a linker is present between the cytidine or adenosine deaminase and the napDNAbp. In some embodiments, cytidine or adenosine deaminase and the napDNAbp are fused via any of the linkers provided herein. For example, in some embodiments the cytidine or adenosine deaminase and the napDNAbp are fused via any of the linkers provided herein.

It should be appreciated that the fusion proteins or complexes of the present disclosure may comprise one or more additional features. For example, in some embodiments, the fusion protein or complex may comprise inhibitors, cytoplasmic localization sequences, export sequences, such as nuclear export sequences, or other localization sequences, as well as sequence tags that are useful for solubilization, purification, or detection of the fusion proteins or complexes. Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FLASH tags, V5 tags, and SBP-tags. Additional suitable sequences will be apparent to those of skill in the art. In some embodiments, the fusion protein or complex comprises one or more His tags.

Exemplary, yet nonlimiting, fusion proteins are described in International PCT Application Nos. PCT/US2017/045381, PCT/US2019/044935, and PCT/US2020/016288, each of which is incorporated herein by reference for its entirety.

Fusion Proteins or Complexes with Internal Insertions

Provided herein are fusion proteins or complexes comprising a heterologous polypeptide fused to a nucleic acid programmable nucleic acid binding protein, for example, a napDNAbp. The heterologous polypeptide can be fused to the napDNAbp at a C-terminal end of the napDNAbp, an N-terminal end of the napDNAbp, or inserted at an internal location of the napDNAbp. In some embodiments, the heterologous polypeptide is a deaminase (e.g., cytidine or adenosine deaminase) or a functional fragment thereof. For example, a fusion protein can comprise a deaminase flanked by an N-terminal fragment and a C-terminal fragment of a Cas9 or Cas12 (e.g., Cas12b/C₂cl), polypeptide.

The deaminase can be a circular permutant deaminase. In some embodiments, the deaminase is a circular permutant TadA, circularly permutated at amino acid residue 116, 136, or 65 as numbered in a TadA reference sequence.

The fusion protein or complexes can comprise more than one deaminase. The fusion protein or complex can comprise, for example, 1, 2, 3, 4, 5 or more deaminases. The deaminases in a fusion protein or complex can be adenosine deaminases, cytidine deaminases, or a combination thereof.

In some embodiments, the napDNAbp in the fusion protein or complex contains a Cas9 polypeptide or a fragment thereof. The Cas9 polypeptide can be a variant Cas9 polypeptide. The Cas9 polypeptide can be a circularly permuted Cas9 protein.

The heterologous polypeptide (e.g., deaminase) can be inserted in the napDNAbp (e.g., Cas9 or Cas12 (e.g., Cas12b/C2c1)) at a suitable location, for example, such that the napDNAbp retains its ability to bind the target polynucleotide and a guide nucleic acid. A deaminase (e.g., adenosine deaminase, cytidine deaminase, or adenosine deaminase and cytidine deaminase (dual deaminase)) can be inserted into a napDNAbp without compromising function of the deaminase (e.g., base editing activity) or the napDNAbp (e.g., ability to bind to target nucleic acid and guide nucleic acid).

In some embodiments, the deaminase (e.g., adenosine deaminase, cytidine deaminase, or adenosine deaminase and cytidine deaminase) is inserted in regions of the Cas9 polypeptide comprising higher than average B-factors (e.g., higher B factors compared to the total protein or the protein domain comprising the disordered region). Cas9 polypeptide positions comprising a higher than average B-factor can include, for example, residues 768, 792, 1052, 1015, 1022, 1026, 1029, 1067, 1040, 1054, 1068, 1246, 1247, and 1248 as numbered in the above Cas9 reference sequence. Cas9 polypeptide regions comprising a higher than average B-factor can include, for example, residues 792-872, 792-906, and 2-791 as numbered in the above Cas9 reference sequence.

In some embodiments, a heterologous polypeptide (e.g., deaminase) is inserted in a flexible loop of a Cas9 polypeptide. The flexible loop portions can be selected from the group consisting of 530-537, 569-570, 686-691, 943-947, 1002-1025, 1052-1077, 1232-1247, or 1298-1300 as numbered in the above Cas9 reference sequence, or a corresponding amino acid residue in another Cas9 polypeptide. The flexible loop portions can be selected from the group consisting of: 1-529, 538-568, 580-685, 692-942, 948-1001, 1026-1051, 1078-1231, or 1248-1297 as numbered in the above Cas9 reference sequence, or a corresponding amino acid residue in another Cas9 polypeptide.

A heterologous polypeptide (e.g., adenine deaminase) can be inserted into a Cas9 polypeptide region corresponding to amino acid residues: 1017-1069, 1242-1247, 1052-1056, 1060-1077, 1002-1003, 943-947, 530-537, 568-579, 686-691, 1242-1247, 1298-1300, 1066-1077, 1052-1056, or 1060-1077 as numbered in the above Cas9 reference sequence, or a corresponding amino acid residue in another Cas9 polypeptide.

A heterologous polypeptide (e.g., adenine deaminase) can be inserted in place of a deleted region of a Cas9 polypeptide. The deleted region can correspond to an N-terminal or C-terminal portion of the Cas9 polypeptide. Exemplary internal fusions base editors are provided in Table 4A below:

TABLE 4A
Insertion loci in Cas9 proteins

| BE ID | Modification | Other ID |
|---|---|---|
| IBE001 | Cas9 TadA ins 1015 | ISLAY01 |
| IBE002 | Cas9 TadA ins 1022 | ISLAY02 |
| IBE003 | Cas9 TadA ins 1029 | ISLAY03 |
| IBE004 | Cas9 TadA ins 1040 | ISLAY04 |
| IBE005 | Cas9 TadA ins 1068 | ISLAY05 |
| IBE006 | Cas9 TadA ins 1247 | ISLAY06 |
| IBE007 | Cas9 TadA ins 1054 | ISLAY07 |
| IBE008 | Cas9 TadA ins 1026 | ISLAY08 |
| IBE009 | Cas9 TadA ins 768 | ISLAY09 |
| IBE020 | delta HNH TadA 792 | ISLAY20 |
| IBE021 | N-term fusion single TadA helix truncated 165-end | ISLAY21 |
| IBE029 | TadA-Circular Permutant116 ins1067 | ISLAY29 |
| IBE031 | TadA- Circular Permutant 136 ins1248 | ISLAY31 |
| IBE032 | TadA- Circular Permutant 136ins 1052 | ISLAY32 |
| IBE035 | delta 792-872 TadA ins | ISLAY35 |
| IBE036 | delta 792-906 TadA ins | ISLAY36 |
| IBE043 | TadA-Circular Permutant 65 ins1246 | ISLAY43 |
| IBE044 | TadA ins C-term truncate2 791 | ISLAY44 |

A heterologous polypeptide (e.g., deaminase) can be inserted within a structural or functional domain of a Cas9 polypeptide. A heterologous polypeptide (e.g., deaminase) can be inserted between two structural or functional domains of a Cas9 polypeptide. A heterologous polypeptide (e.g., deaminase) can be inserted in place of a structural or functional domain of a Cas9 polypeptide, for example, after deleting the domain from the Cas9 polypeptide. The structural or functional domains of a Cas9 polypeptide can include, for example, RuvC I, RuvC II, RuvC III, Rec1, Rec2, PI, or HNH.

A fusion protein can comprise a linker between the deaminase and the napDNAbp polypeptide. The linker can be a peptide or a non-peptide linker. For example, the linker can be an XTEN, (GGGS) n (SEQ ID NO: 246), SGGSSGGS(SEQ ID NO: 330), (GGGGS) n (SEQ ID NO: 247), (G) n, (EAAAK) n (SEQ ID NO: 248), (GGS) n, SGSETPGTSESATPES(SEQ ID NO: 249). In some embodiments, the fusion protein comprises a linker between the N-terminal Cas9 fragment and the deaminase. In some embodiments, the fusion protein comprises a linker between the C-terminal Cas9 fragment and the deaminase. In some embodiments, the N-terminal and C-terminal fragments of napDNAbp are connected to the deaminase with a linker. In some embodiments, the N-terminal and C-terminal fragments are joined to the deaminase domain without a linker. In some embodiments, the fusion protein comprises a linker between the N-terminal Cas9 fragment and the deaminase but does not comprise a linker between the C-terminal Cas9 fragment and the deaminase. In some embodiments, the fusion protein comprises a linker between the C-terminal Cas9 fragment and the deaminase but does not comprise a linker between the N-terminal Cas9 fragment and the deaminase.

In some embodiments, the napDNAbp in the fusion protein or complex is a Cas12 polypeptide, e.g., Cas12b/C2c1, or a functional fragment thereof capable of associating with a nucleic acid (e.g., a gRNA) that guides the Cas 12 to a specific nucleic acid sequence. The Cas12 polypeptide can be a variant Cas 12 polypeptide. In other embodiments, the N- or C-terminal fragments of the Cas 12 polypeptide comprise a nucleic acid programmable DNA binding domain or a RuvC domain. In other embodiments, the fusion protein contains a linker between the Cas12 polypeptide and the catalytic domain. In other embodiments, the amino acid sequence of the linker is GGSGGS(SEQ ID NO: 250) or GSSGSETPGTSESATPESSG (SEQ ID NO: 251). In other embodiments, the linker is a rigid linker. In other embodiments of the above aspects, the linker is encoded by GGAGGCTCTGGAGGAAGC(SEQ ID NO: 252) or GGCTCTTCTGGATCTGAAACACCTGGCACAAGCGAGAGCGCCACCCCTGAGAGCTCTGGC (SEQ ID NO: 253).

In other embodiments, the fusion protein or complex contains a nuclear localization signal (e.g., a bipartite nuclear localization signal). In other embodiments, the amino acid sequence of the nuclear localization signal is MAPKKKRKVGIHGVPAA (SEQ ID NO: 261). In other embodiments of the above aspects, the nuclear localization signal is encoded by the following sequence:

ATGGCCCCAAAGAAGAAGCGGAAGGTCGGTATCCACGGAGTCCCAGCAGCC(SEQ ID NO: 262). In other embodiments, the Cas12b polypeptide contains a mutation that silences the catalytic activity of a RuvC domain. In other embodiments, the Cas12b polypeptide contains D574A, D829A and/or D952A mutations.

In some embodiments, the fusion protein or complex comprises a napDNAbp domain (e.g., Cas12-derived domain) with an internally fused nucleobase editing domain (e.g., all or a portion (e.g., a functional portion) of a deaminase domain, e.g., an adenosine deaminase domain). In some embodiments, the napDNAbp is a Cas12b. In some embodiments, the base editor comprises a BhCas12b domain with an internally fused TadA*8 domain inserted at the loci provided in Table 4B below.

TABLE 4B
Insertion loci in Cas12b proteins

| | Insertion | Inserted |
|---|---|---|
| | site | between aa |
| BhCas12b | | |
| position 1 | 153 | PS |
| position 2 | 255 | KE |
| position 3 | 306 | DE |
| position 4 | 980 | DG |
| position 5 | 1019 | KL |
| position 6 | 534 | FP |
| position 7 | 604 | KG |
| position 8 | 344 | HF |
| BvCas12b | | |
| position 1 | 147 | PD |
| position 2 | 248 | GG |
| position 3 | 299 | PE |
| position 4 | 991 | GE |
| position 5 | 1031 | KM |
| AaCas12b | | |
| position 1 | 157 | PG |
| position 2 | 258 | VG |
| position 3 | 310 | DP |
| position 4 | 1008 | GE |
| position 5 | 1044 | GK |

In some embodiments, the base editing system described herein is an ABE with TadA inserted into a Cas9. Polypeptide sequences of relevant ABEs with TadA inserted into a Cas9 are provided in the attached Sequence Listing as SEQ ID NOs: 263-308.

Exemplary, yet nonlimiting, fusion proteins are described in International PCT Application Nos. PCT/US2020/016285 and U.S. Provisional Application Nos. 62/852,228 and 62/852,224, the contents of which are incorporated by reference herein in their entireties.

### A to G Editing

In some embodiments, a base editor described herein comprises an adenosine deaminase domain. Such an adenosine deaminase domain of a base editor can facilitate the editing of an adenine (A) nucleobase to a guanine (G) nucleobase by deaminating the A to form inosine (I), which exhibits base pairing properties of G. In some embodiments, an A-to-G base editor further comprises an inhibitor of inosine base excision repair, for example, a uracil glycosylase inhibitor (UGI) domain or a catalytically inactive inosine specific nuclease. Without wishing to be bound by any particular theory, the UGI domain or catalytically inactive inosine specific nuclease can inhibit or prevent base excision repair of a deaminated adenosine residue (e.g., inosine), which can improve the activity or efficiency of the base editor.

A base editor comprising an adenosine deaminase can act on any polynucleotide, including DNA, RNA and DNA-RNA hybrids. In an embodiment an adenosine deaminase domain of a base editor comprises all or a portion (e.g., a functional portion) of an ADAT comprising one or more mutations which permit the ADAT to deaminate a target A in DNA. For example, the base editor can comprise all or a portion (e.g., a functional portion) of an ADAT from Escherichia coli (EcTadA) comprising one or more of the following mutations: D108N, A¹⁰⁶V, D147Y, E155V, L⁸⁴F, H123Y, 1156F, or a corresponding mutation in another adenosine deaminase. Exemplary ADAT homolog polypeptide sequences are provided in the Sequence Listing as SEQ ID NOs: 1 and 309-315.

The adenosine deaminase can be derived from any suitable organism (e.g., E. coli). In some embodiments, the adenosine deaminase is from Escherichia coli, Staphylococcus aureus, Salmonella typhi, Shewanella putrefaciens, Haemophilus influenzae, Caulobacter crescentus, or Bacillus subtilis. In some embodiments, the adenine deaminase is a naturally-occurring adenosine deaminase that includes one or more mutations corresponding to any of the mutations provided herein (e.g., mutations in ecTadA). The corresponding residue in any homologous protein can be identified by e.g., sequence alignment and determination of homologous residues. The mutations in any naturally-occurring adenosine deaminase (e.g., having homology to ecTadA) that correspond to any of the mutations described herein (e.g., any of the mutations identified in ecTadA) can be generated accordingly.

In some embodiments, the adenosine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth in any of the adenosine deaminases provided herein. It should be appreciated that adenosine deaminases provided herein may include one or more mutations (e.g., any of the mutations provided herein). The disclosure provides any deaminase domains with a certain percent identify plus any of the mutations or combinations thereof described herein. In some embodiments, the adenosine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to a reference sequence, or any of the adenosine deaminases provided herein.

It should be appreciated that any of the mutations provided herein (e.g., based on a TadA reference sequence, such as TadA*7.10 (SEQ ID NO: 1)) can be introduced into other adenosine deaminases, such as E. coli TadA (ecTadA), S. aureus TadA (saTadA), or other adenosine deaminases (e.g., bacterial adenosine deaminases). In some embodiments, the TadA reference sequence is TadA*7.10 (SEQ ID NO: 1). It would be apparent to the skilled artisan that additional deaminases may similarly be aligned to identify homologous amino acid residues that can be mutated as provided herein. Thus, any of the mutations identified in a TadA reference sequence can be made in other adenosine deaminases (e.g., ecTada) that have homologous amino acid residues. It should also be appreciated that any of the mutations provided herein can be made individually or in any combination in a TadA reference sequence or another adenosine deaminase.

In some embodiments, the adenosine deaminase comprises an alteration or set of alterations selected from those listed in Tables 5A-5E below:

TABLE 5A
Adenosine Deaminase Variants. Residue positions in the E. coli TadA variant
(TadA*) are indicated.

| | 23 | 26 | 36 | 37 | 48 | 49 | 51 | 72 | 84 | 87 | 106 | 108 | 123 | 125 | 142 | 146 | 147 | 152 | 155 | 156 | 157 | 161 |
|---|---|---|---|---|---|---|---|---|---|---|---|---|---|---|---|---|---|---|---|---|---|---|
| TadA*0.1 | W | R | H | N | P | | R | N | L | S | A | D | H | G | A | S | D | R | E | I | K | K |
| TadA*0.2 | W | R | H | N | P | | R | N | L | S | A | D | H | G | A | S | D | R | E | I | K | K |
| TadA*1.1 | W | R | H | N | P | | R | N | L | S | A | N | H | G | A | S | D | R | E | I | K | K |
| TadA*1.2 | W | R | H | N | P | | R | N | L | S | V | N | H | G | A | S | D | R | E | I | K | K |
| TadA*2.1 | W | R | H | N | P | | R | N | L | S | V | N | H | G | A | S | Y | R | V | I | K | K |
| TadA*2.2 | W | R | H | N | P | | R | N | L | S | V | N | H | G | A | S | Y | R | V | I | K | K |
| TadA*2.3 | W | R | H | N | P | | R | N | L | S | V | N | H | G | A | S | Y | R | V | I | K | K |
| TadA*2.4 | W | R | H | N | P | | R | N | L | S | V | N | H | G | A | S | Y | R | V | I | K | K |
| TadA*2.5 | W | R | H | N | P | | R | N | L | S | V | N | H | G | A | S | Y | R | V | I | K | K |
| TadA*2.6 | W | R | H | N | P | | R | N | L | S | V | N | H | G | A | S | Y | R | V | I | K | K |
| TadA*2.7 | W | R | H | N | P | | R | N | L | S | V | N | H | G | A | S | Y | R | V | I | K | K |
| TadA*2.8 | W | R | H | N | P | | R | N | L | S | V | N | H | G | A | S | Y | R | V | I | K | K |
| TadA*2.9 | W | R | H | N | P | | R | N | L | S | V | N | H | G | A | S | Y | R | V | I | K | K |
| TadA*2.10 | W | R | H | N | P | | R | N | L | S | V | N | H | G | A | S | Y | R | V | I | K | K |
| TadA*2.11 | W | R | H | N | P | | R | N | L | S | V | N | H | G | A | S | Y | R | V | I | K | K |
| TadA*2.12 | W | R | H | N | P | | R | N | L | S | V | N | H | G | A | S | Y | R | V | I | K | K |
| TadA*3.1 | W | R | H | N | P | | R | N | F | S | V | N | Y | G | A | S | Y | R | V | F | K | K |
| TadA*3.2 | W | R | H | N | P | | R | N | F | S | V | N | Y | G | A | S | Y | R | V | F | K | K |
| TadA*3.3 | W | R | H | N | P | | R | N | F | S | V | N | Y | G | A | S | Y | R | V | F | K | K |
| TadA*3.4 | W | R | H | N | P | | R | N | F | S | V | N | Y | G | A | S | Y | R | V | F | K | K |
| TadA*3.5 | W | R | H | N | P | | R | N | F | S | V | N | Y | G | A | S | Y | R | V | F | K | K |
| TadA*3.6 | W | R | H | N | P | | R | N | F | S | V | N | Y | G | A | S | Y | R | V | F | K | K |
| TadA*3.7 | W | R | H | N | P | | R | N | F | S | V | N | Y | G | A | S | Y | R | V | F | K | K |
| TadA*3.8 | W | R | H | N | P | | R | N | F | S | V | N | Y | G | A | S | Y | R | V | F | K | K |
| TadA*4.1 | W | R | H | N | P | | R | N | L | S | V | N | H | G | N | S | Y | R | V | I | K | K |
| TadA*4.2 | W | G | H | N | P | | R | N | L | S | V | N | H | G | N | S | Y | R | V | I | K | K |
| TadA*4.3 | W | R | H | N | P | | R | N | F | S | V | N | Y | G | N | S | Y | R | V | F | K | K |
| TadA*5.1 | W | R | L | N | P | | L | N | F | S | V | N | Y | G | A | C | Y | R | V | F | N | K |
| TadA*5.2 | W | R | H | S | P | | R | N | F | S | V | N | Y | G | A | S | Y | R | V | F | K | T |
| TadA*5.3 | W | R | L | N | P | | L | N | I | S | V | N | Y | G | A | C | Y | R | V | F | N | K |
| TadA*5.4 | W | R | H | S | P | | R | N | F | S | V | N | Y | G | A | S | Y | R | V | F | K | T |
| TadA*5.5 | W | R | L | N | P | | L | N | F | S | V | N | Y | G | A | C | Y | R | V | F | N | K |
| TadA*5.6 | W | R | L | N | P | | L | N | F | S | V | N | Y | G | A | C | Y | R | V | F | N | K |
| TadA*5.7 | W | R | L | N | P | | L | N | F | S | V | N | Y | G | A | C | Y | R | V | F | N | K |
| TadA*5.8 | W | R | L | N | P | | L | N | F | S | V | N | Y | G | A | C | Y | R | V | F | N | K |
| TadA*5.9 | W | R | L | N | P | | L | N | F | S | V | N | Y | G | A | C | Y | R | V | F | N | K |
| TadA*5.10 | W | R | L | N | P | | L | N | F | S | V | N | Y | G | A | C | Y | R | V | F | N | K |
| TadA*5.11 | W | R | L | N | P | | L | N | F | S | V | N | Y | G | A | C | Y | R | V | F | N | K |
| TadA*5.12 | W | R | L | N | P | | L | N | F | S | V | N | Y | G | A | C | Y | R | V | F | N | K |
| TadA*5.13 | W | R | H | N | P | | L | D | F | S | V | N | Y | A | A | S | Y | R | V | F | K | K |
| TadA*5.14 | W | R | H | N | S | | L | N | F | C | V | N | Y | G | A | S | Y | R | V | F | K | K |
| TadA*6.1 | W | R | H | N | S | | L | N | F | S | V | N | Y | G | N | S | Y | R | V | F | K | K |
| TadA*6.2 | W | R | H | N | T | V | L | N | F | S | V | N | Y | G | N | S | Y | R | V | F | N | K |
| TadA*6.3 | W | R | L | N | S | | L | N | F | S | V | N | Y | G | A | C | Y | R | V | F | N | K |
| TadA*6.4 | W | R | L | N | S | | L | N | F | S | V | N | Y | G | N | C | Y | R | V | F | N | K |
| TadA*6.5 | W | R | L | N | T | V | L | N | F | S | V | N | Y | G | A | C | Y | R | V | F | N | K |
| TadA*6.6 | W | R | L | N | T | V | L | N | F | S | V | N | Y | G | N | C | Y | R | V | F | N | K |
| TadA*7.1 | W | R | L | N | A | | L | N | F | S | V | N | Y | G | A | C | Y | R | V | F | N | K |
| TadA*7.2 | W | R | L | N | A | | L | N | F | S | V | N | Y | G | N | C | Y | R | V | F | N | K |
| TadA*7.3 | L | R | L | N | A | | L | N | F | S | V | N | Y | G | A | C | Y | R | V | F | N | K |
| TadA*7.4 | R | R | L | N | A | | L | N | F | S | V | N | Y | G | A | C | Y | R | V | F | N | K |
| TadA*7.5 | W | R | L | N | A | | L | N | F | S | V | N | Y | G | A | C | Y | H | V | F | N | K |
| TadA*7.6 | W | R | L | N | A | | L | N | I | S | V | N | Y | G | A | C | Y | P | V | F | N | K |
| TadA*7.7 | L | R | L | N | A | | L | N | F | S | V | N | Y | G | A | C | Y | P | V | F | N | K |
| TadA*7.8 | L | R | L | N | A | | L | N | F | S | V | N | Y | G | N | C | Y | R | V | F | N | K |
| TadA*7.9 | L | R | L | N | A | | L | N | F | S | V | N | Y | G | N | C | Y | P | V | F | N | K |
| TadA*7.10 | R | R | L | N | A | | L | N | F | S | V | N | Y | G | A | C | Y | P | V | F | N | K |

TABLE 5B
TadA*8 Adenosine Deaminase Variants. Residue positions in the E. coli TadA
variant (TadA*) are indicated. Alterations are referenced to TadA*7.10 (first row).

| | 23 | 63 | 84 | 51 | 76 | 82 | 84 | 106 | 108 | 123 | 146 | 147 | 152 | 154 | 155 | 156 | 157 | 166 |
|---|---|---|---|---|---|---|---|---|---|---|---|---|---|---|---|---|---|---|
| TadA*7.10 | R | L | A | L | I | V | F | V | N | Y | C | Y | P | Q | V | F | N | T |
| TadA*8.1 | | | | | | | | | | | | T | | | | | | |
| TadA*8.2 | | | | | | | | | | | | R | | | | | | |
| TadA*8.3 | | | | | | | | | | | | | | S | | | | |
| TadA*8.4 | | | | | | | | | | H | | | | | | | | |
| TadA*8.5 | | | | | | S | | | | | | | | | | | | |
| TadA*8.6 | | | | | | | | | | | | | | | | | | R |
| TadA*8.7 | | | | | | | | | | | | | | R | | | | |
| TadA*8.8 | | | | | | | | | | H | | R | | R | | | | |
| TadA*8.9 | | | | | Y | | | | | | | R | | R | | | | |
| TadA*8.10 | | | | | | | | | | | | R | | R | | | | R |
| TadA*8.11 | | | | | | | | | | | | T | | R | | | | |
| TadA*8.12 | | | | | | | | | | | | T | | S | | | | |
| TadA*7.10 | R | L | A | L | I | V | F | V | N | Y | C | Y | P | Q | V | F | N | T |
| TadA*8.13 | | | | | Y | | | | | H | | R | | R | | | | |
| TadA*8.14 | | | | | Y | S | | | | | | | | | | | | |
| TadA*8.15 | | | | | | S | | | | | | R | | | | | | |
| TadA*8.16 | | | | | | S | | | | H | | R | | | | | | |
| TadA*8.17 | | | | | | S | | | | | | | | R | | | | |
| TadA*8.18 | | | | | | S | | | | H | | | | R | | | | |
| TadA*8.19 | | | | | | S | | | | H | | R | | R | | | | |
| TadA*8.20 | | | | | Y | S | | | | H | | R | | R | | | | |
| TadA*8.21 | | | | | | | | | | | | R | | S | | | | |
| TadA*8.22 | | | | | | S | | | | | | | | S | | | | |
| TadA*8.23 | | | | | | S | | | | H | | | | | | | | |
| TadA*8.24 | | | | | | S | | | | H | | T | | | | | | |

TABLE 5C
TadA*9 Adenosine Deaminase Variants. Alterations are referenced
to TadA*7.10. Additional details of TadA*9 adenosine
deaminases are described in International PCT Application
No. PCT/US2020/049975, which is incorporated herein by
reference in its entirety for all purposes.

| TadA*9 | |
|---|---|
| Description | Alterations |
| TadA*9.1 | E25F, V82S, Y123H, T133K, Y147R, Q154R |
| TadA*9.2 | E25F, V82S, Y123H, Y147R, Q154R |
| TadA*9.3 | V82S, Y123H, P124W, Y147R, Q154R |
| TadA*9.4 | L51W, V82S, Y123H, C146R, Y147R, Q154R |
| TadA*9.5 | P54C, V82S, Y123H, Y147R, Q154R |
| TadA*9.6 | Y73S, V82S, Y123H, Y147R, Q154R |
| TadA*9.7 | N38G, V82T, Y123H, Y147R, Q154R |
| TadA*9.8 | R23H, V82S, Y123H, Y147R, Q154R |
| TadA*9.9 | R21N, V82S, Y123H, Y147R, Q154R |
| TadA*9.10 | V82S, Y123H, Y147R, Q154R, A158K |
| TadA*9.11 | N72K, V82S, Y123H, D139L, Y147R, Q154R, |
| TadA*9.12 | E25F, V82S, Y123H, D139M, Y147R, Q154R |
| TadA*9.13 | M70V, V82S, M94V, Y123H, Y147R, Q154R |
| TadA*9.14 | Q71M, V82S, Y123H, Y147R, Q154R |
| TadA*9.15 | E25F, V82S, Y123H, T133K, Y147R, Q154R |
| TadA*9.16 | E25F, V82S, Y123H, Y147R, Q154R |
| TadA*9.17 | V82S, Y123H, P124W, Y147R, Q154R |
| TadA*9.18 | L51W, V82S, Y123H, C146R, Y147R, Q154R |
| TadA*9.19 | P54C, V82S, Y123H, Y147R, Q154R |
| TadA*9.2 | Y73S, V82S, Y123H, Y147R, Q154R |
| TadA*9.21 | N38G, V82T, Y123H, Y147R, Q154R |
| TadA*9.22 | R23H, V82S, Y123H, Y147R, Q154R |
| TadA*9.23 | R21N, V82S, Y123H, Y147R, Q154R |
| TadA*9.24 | V82S, Y123H, Y147R, Q154R, A158K |
| TadA*9.25 | N72K, V82S, Y123H, D139L, Y147R, Q154R, |
| TadA*9.26 | E25F, V82S, Y123H, D139M, Y147R, Q154R |
| TadA*9.27 | M70V, V82S, M94V, Y123H, Y147R, Q154R |
| TadA*9.28 | Q71M, V82S, Y123H, Y147R, Q154R |
| TadA*9.29 | E25F_I76Y_V82S_Y123H_Y147R_Q154R |
| TadA*9.30 | I76Y_V82T_Y123H_Y147R_Q154R |
| TadA*9.31 | N38G_I76Y_V82S_Y123H_Y147R_Q154R |
| TadA*9.32 | N38G_I76Y_V82T_Y123H_Y147R_Q154R |
| TadA*9.33 | R23H_I76Y_V82S_Y123H_Y147R_Q154R |
| TadA*9.34 | P54C_I76Y_V82S_Y123H_Y147R_Q154R |
| TadA*9.35 | R21N_I76Y_V82S_Y123H_Y147R_Q154R |
| TadA*9.36 | I76Y_V82S_Y123H_D138M_Y147R_Q154R |
| TadA*9.37 | Y72S_I76Y_V82S_Y123H_Y147R_Q154R |
| TadA*9.38 | E25F_I76Y_V82S_Y123H_Y147R_Q154R |
| TadA*9.39 | I76Y_V82T_Y123H_Y147R_Q154R |
| TadA*9.40 | N38G_I76Y_V82S_Y123H_Y147R_Q154R |
| TadA*9.41 | N38G_I76Y_V82T_Y123H_Y147R_Q154R |
| TadA*9.42 | R23H_I76Y_V82S_Y123H_Y147R_Q154R |
| TadA*9.43 | P54C_I76Y_V82S_Y123H_Y147R_Q154R |
| TadA*9.44 | R21N_I76Y_V82S_Y123H_Y147R_Q154R |
| TadA*9.45 | I76Y_V82S_Y123H_D138M_Y147R_Q154R |
| TadA*9.46 | Y72S_I76Y_V82S_Y123H_Y147R_Q154R |
| TadA*9.47 | N72K_V82S, Y123H, Y147R, Q154R |
| TadA*9.48 | Q71M_V82S, Y123H, Y147R, Q154R |
| TadA*9.49 | M70V, V82S, M94V, Y123H, Y147R, Q154R |
| TadA*9.50 | V82S, Y123H, T133K, Y147R, Q154R |
| TadA*9.51 | V82S, Y123H, T133K, Y147R, Q154R, A158K |
| TadA*9.52 | M70V, Q71M, N72K, V82S, Y123H, Y147R, Q154R |
| TadA*9.53 | N72K_V82S, Y123H, Y147R, Q154R |
| TadA*9.54 | Q71M_V82S, Y123H, Y147R, Q154R |
| TadA*9.55 | M70V, V82S, M94V, Y123H, Y147R, Q154R |
| TadA*9.56 | V82S, Y123H, T133K, Y147R, Q154R |
| TadA*9.57 | V82S, Y123H, T133K, Y147R, Q154R, A158K |
| TadA*9.58 | M70V, Q71M, N72K, V82S, Y123H, Y147R, Q154R |

In some embodiments, the adenosine deaminase comprises a TadA*8.20 adenosine deaminase variant further comprising an F149Y amino acid alteration. In some embodiments, the adenosine deaminase comprises a TadA*8.20 adenosine deaminase variant further comprising the amino acid alterations R147D, F149Y, T166I, and D167N(TadA*8.10+). In some embodiments, the adenosine deaminase comprises a TadA*8.20 adenosine deaminase variant further comprising the amino acid alterations S82T and F149Y (TadA*9v1). In some embodiments, the adenosine deaminase comprises a TadA*8.20 adenosine deaminase variant further comprising the amino acid alterations Y147D, F149Y, T166I, D167N and S82T (TadA*9v2).

In some embodiments, the adenosine deaminase comprises one or more of MII, MIS, S2A, S2E, S2H, S2R, S2L, E3L, V4D, V4E, V4M, V4K, V4S, V4T, V4A, E5K, F6S, F6G, F6H, F6Y, F6I, F6E, S7K, H8E, H8Y, H8H, H8Q, H8E, H8G, H8S, E9Y, E9K, E9V, E9E, Y10F, Y10W, Y10Y, M12S, M12L, M12R, M12W, R13H, R13I, R13Y, R13R, R13G, R13S, H14N, A15D, A15V, A15L, A15H, T17T, T17A, T17W, T17L, T17F, T17R, T17S, L18A, L18E, L18N, L18L, L18S, A19N, A19H, A19K, A19A, A19D, A19G, A19M, R2IN, K20K, K20A, K20R, K20E, K20G, K20C, K20Q R21A, R21R, R21N, R21Y, R21C G22P, A22W, A22R, W23D, R23H, W23G, W23Q, W23L, W23R, W23H W23D W23M, W23W, W23I, D24E, D24G, D24W, D24D, D24R, E25F, E25M, E25D, E25A, E25G, E25R, E25E, E25H E25V, E25S, E25Y, R26D, R26E, R26G, R26N, R26Q, R26C, R26L, R26K, R26W, R26C, R26P, R26R, R26A, R26H, E27E, E27Q, E27H, E27C, E27G, E27K, E27S, E27P, E27R, E27L, E27V, E27D, V28V, V28A, V28C, V28G, V28P, V28S, V28T, P29V, P29P, P29A, P29G, P29K, P29L, V30V, V30I, V30L, V30F, V30G, V30A, V30M, L34S, L34V, L34L, L34M, L34W, L34G, H36E, H36V, L36H, H36L, H36N, N37N, N37H, N37R, N37T, N37S, N38G, N38R, N38N, N38E, V40I, W45A, W45W, W45R, W45L, W45N, N46N, N46M, N46P, N46G, N46L, N46R, N46V, R46W, R46F, R46Q, R46M, R47A, R47Q, R47F, R47K, R47P, R47W, R47M, R47R, R47G, R47S, R47V, R47H, P48T, P48L, P48A, P48I, P48S, P48R, P48K, P48D, P48E, P48H, P48G, P48P, P48N, 149G, 149H, 149V, I49F, 149H, 491, 149M, 149N, 149K, 149Q, 149T, G50L, G50S, G50R, G50G, R51H, R51L, R51N, L51W, R51Y, R51G, R51V, R51R, H52D, H52Y, H521, H52H, D53D, D53E, D53G, D53P, P54C, P54T, P54P, P54E, A55H, T55A, T55I, T55V, T55G, T55T, A56A, A56H, A56W, A56E, A56S, H57P, H57A, H57H, H57N, A58G, A58E, A58A, A58R, E59A, E59G, E59I, E59Q, E59W, E59E, E59T, E59H, E59P, M61A, M61I, M61L, M61V, M61P, M61G, M61I, L63S, L63V, L63T, L63R, L63H, L63A, R64A, R64Q, R64R, R64D, Q65V, Q65H, Q65G, Q65P, Q65F, Q65Q, Q65R, G66V, G66E, G66T, G66G, G66C, G67G, G67W, G67I, G67A, G67D, G67L, G67V, L68Q, L68M, L68V, L68H, L68L, L68G,V69A, V69M, V69V, M70V, M70L, E70A, M70A, M70M, M70E, M70T, M70v, Q71M, Q7IN, Q71L, Q71R, Q71Q, Q71I, N72A, N72K, N72S, N72D, N72Y, N72N, N72H, N72G, N72M, Y73G, Y73I, Y73K, Y73R, Y73S, Y73Y, Y73H, Y73A, R74A, R74Q, R74G, R74K, R74L, R74N, R74G, R74K, R74R, I76H, I76R, 176W, 176Y, 176V, 176Q, I76L, 176D, 176F, 176I, 176N, I76T, I76Y, D77G, D77D, D77A, D77Q, A78Y, A78T, A78G, A78A, A78I, T79M, T79R, T79L, T79T, L80M, L80Y, L80I, L80V, L80L, Y81D, Y81V, Y81Y, Y81M, V82A, V82S, V82G, V82T, V82V, V82Q, V82Y, T83L, T83F, T83T, T83N, L84E, L84F, L84Y, L84I, L84L, L84M, L84A, L84T, L84S, E85K, E85G, E85P, E85S, E85E, E85F, E85V, E85R, P86T, P86C, P86P, P86L, P86N, P86K, P86H, C87M, C87I, C87S, C87N, C87P, S87C, S87L, S87V, V88A, V88M, V88V, V88T, V88E, V88D, V88S, C90S, C90P, C90A, C90T, C90M, A91A, A91G, A91S, A91V, A91T, A91C, A91L, G92T, G92M, G92A, G92Y, G92G, A93I, A93C, A93M, A93V, A93A, M94M, M94T, M94A, M94V, M94L, M94I, M94H, 195S, 195G, 195L, 195H, 195V, H96A, H96L, H96R, H96S, H96H, H96N, H96E, S97C, S97G, S97I, S97M, S97R, S97S, S97P, R98K, R981, R98N, R98Q, R98G, R98H, R98C, R98L, R98R, G100R, G100V, G100K, G100A, G100S, G100M, G100I, R101V, R101R, R101S, R101C, V102A, V102F, V102I, V102V, D103A, V103A, V103G, V103F, V103V, F104G, D104N, F104V, F104I, F104L, F104A, F104F, F104R, G105V, G105W, G105G, G105M, G105A, A106T, V106Q, V106F, V106W, V106M, A106A, A106Q, A106F, A106G, A106W, A106M, A106V, A106R, A106L, A106S, A106B, A106I, R107C, R107G, R107P, R107K, R107A, R107N, R107W, R107H, R107S, R107R, R107F, D108N, D108F, D108G, D108V, D108A, D108Y, D108H, D108I, D108K, D108L, D108M, D108Q, N108Q, N108F, N108W, N108M, N108K, D108K, D108F, D108M, D108Q, D108R, D108W, D108S, D108E, D108T, D108R, D108D, A109H, A109K, A109R, A109S, A109T, A109V, A109A, A109D, K110G, K110H, K110I, K110R, K110T, K110K, K110A, K1101, T111A, T111G, T111H, T111R, T111T, T111K, G112A, G112G, G112H, G112T, G112R, A113N, A114G, A114H, A114V, A114C, A114S, A114A, G115S, G115G, G115M, G115L, G115A, G115F, L117M, L117L, L117W, L117A, L117S, L117N, L117V, M118D, M118G, M118K, M118N, M118V, M118M, M118L, M118R, D119L, D119N, D119S, D119V, D119D, V120H, V120L, V120V, V120T, V120A, V120E, V120G, V120D, L121D, L121M, L12IN, L121K, L121L, H122H, H122N, H122P, H122R, H122S, H122Y, H122G, H122T, H122L, H123C, H123G, H123P, H123V, H123Y, Y123H, H123Y, H123H, P124P, P124H, P124A, P124Y, P124D, P124G, P124I, P124L, P124W, G125H, G125I, G125A, G125M, G125K, G125G, G125P, M126D, M126H, M126K, M126I, M126N, M1260, M126S, M126Y, M126M, M126G, N127H, N127S, N127D, N127K, N127R, N127N, N127I, N127P, N127M, H128R, H128N, H128L, H128H, R129H, R129Q, R129V, R129I, R129E, R129V, R129R, R129M, R129P, V130R, V130V, V130E, V130D, E131E, E131I, E131V, E131K, I1321, 1132F, I132T, 1132L, 1132V, 1132E, T133V, T133E, T133G, T133K, T133T, T133A, T133H, T133F, T133I, E134A, E134E, E134G, E134I, E134H, E134K, E134T, G135G, G135V, G135I, G135P, G135E, 1136G, 1136L, 1136T, 11361, 1137A, 1137D, 1137E, L137M, I137S, L137L, L1371, A138D, A138E, A138G, S138A, A138N, A138S, A138T, A138V, A138Y, A138A, A138M, A138L, D139E, D139I, D139C, D139L, D139M, D139D, D139G, D139H, D139A, E140A, E140C, E140L, E140R, E140K, E140E, E140D, C141S, C141A, C141C, C141V, C141E, A142N, A142D, A142G, A142A, A142L, A142S, A142T, A142N, A142S, A142V, A142E, A142C, A143D, A143E, A143G, A143D, A143G, A143E, A143L, A143W, A143M, A143S, A143Q, A143R, A143A, A143I, L144S, L144L, L144T, L144A, L145A, L145F, L145G, L145D, L145L, L145C, L145E, L145s, C146R, S146A, S146C, S146D, S146F, S146R, S146T, S146D, S146G, S146S, S146L, D147D, D147L, D147F, D147G, D147Y, Y147T, Y147R, Y147D, D147R, D147Y, D147A, D147T, D147H, D147F, D147U, D147V, D1471, D147C, F148L, F148F, F148R, F148Y, F148A, F148T, F149C, F149M, F149R, F149Y, F149N, F149F, F149A, F149T, F149V R150R, R150M, R150D, R150F, M151F, M151P, M151R, M151V, M151M, M151E, R152C, R152F, R152H, R152P, R152R, R152P, R152Q, R152M, R1520, R153C, R153Q, R153R, R153V, R153E, R153A, R153P, Q154E, Q154H, Q154M, Q154R, Q154L, Q154S, Q154V, Q154Q, Q154F, Q154I, Q154A, Q154K, E155F, E155G, E155I, E155K, E155P, E155V, E155D, E155E, E155L, E155Q, I156V, 1156A, 1156I, 1156L, 1156F, 1156D, 1156K, 1156N, 1156R, 1156Y, E157A, E157F, E157I, E157P, E157T, E157V, N157K, K157N, K157V, K157P, K157I, K157F, K157F, K157T, K157A, K157S, K157R, A158Q, A158K, A158V, A158A, A158D, A158S, A158T, A158N, Q159S, Q159Q, Q159A, Q159F, Q159K, Q159L, Q159N, K160A, K160S, K160E, K160K, K160N, K160F, K160Q, K161T, K161K, K161R, K161I, K161A, K16IN, K161Q, K161S, K161T, A162D, A162Q, R162H, R162P, A162S, A162A, A162N, A162M, A162K, Q163G, Q163S, Q163Q, Q163A, Q163H, Q163N, Q163R, S164F, S164S, S164Q, S164I, S164R, S164Y, S165S, S165P, S165Q, S165A, S165D, S165I, S165T, S165Y, T166T, T166Q, T166E, T166S, T166D, T166K, T166I, T166N, T166P, T166R, D167S D167D, D1671, D167G, D167T, D167A and/or D167N mutation in a TadA reference sequence (e.g., TadA*7.10,ecTadA, or TadA8e), and any alternative mutation at the corresponding position, or one or more corresponding mutations in another adenosine deaminase. Additional mutations are described in U.S. Patent Application Publication No. 2022/0307003 A1 U.S. Pat. No. 11,155,803, and International Patent Application Publications No. WO 2023/288304 A2, PCT/CN2022/143408, WO 2018/027078 A1, WO 2021/158921 A1 and WO 2023/034959 A2, the disclosures of which are incorporated herein by reference in their entirety for all purposes.

In embodiments, a variant of TadA*7.10 comprises one or more alterations selected from any of those alterations provided herein.

In particular embodiments, an adenosine deaminase heterodimer comprises a TadA*8 domain and an adenosine deaminase domain selected from Staphylococcus aureus (S. aureus) TadA, Bacillus subtilis (B. subtilis) TadA, Salmonella typhimurium (S. typhimurium) TadA, Shewanella putrefaciens (S. putrefaciens) TadA, Haemophilus influenzae F3031 (H. influenzae) TadA, Caulobacter crescentus (C. crescentus) TadA, Geobacter sulfurreducens (G. sulfurreducens) TadA, or TadA*7.10.

In some embodiments, the TadA*8 is a variant as shown in Table 5D. Table 5D shows certain amino acid position numbers in the TadA amino acid sequence and the amino acids present in those positions in the TadA-7.10 adenosine deaminase. Table 5D also shows amino acid changes in TadA variants relative to TadA-7.10 following phage-assisted non-continuous evolution (PANCE) and phage-assisted continuous evolution (PACE), as described in M. Richter et al., 2020, Nature Biotechnology, doi.org/10.1038/s41587-020-0453-z, the entire contents of which are incorporated by reference herein. In some embodiments, the TadA*8 is TadA*8a, TadA*8b, TadA*8c, TadA*8d, or TadA*8e. In some embodiments, the TadA*8 is TadA*8e. In one embodiment, an adenosine deaminase is a TadA*8 that comprises or consists essentially of SEQ ID NO: 316 or a fragment thereof having adenosine deaminase activity.

TABLE 5D
Select TadA*8 Variants

| | | TadA amino acid number | | | | | | | | | |
|---|---|---|---|---|---|---|---|---|---|---|---|
| | TadA | 26 | 88 | 109 | 111 | 119 | 122 | 147 | 149 | 166 | 167 |
| | TadA- | R | V | A | T | D | H | Y | F | T | D |
| | 7.10 | | | | | | | | | | |
| PANCE 1 | | | | | R | | | | | | |
| PANCE 2 | | | | S/T | R | | | | | | |
| PACE | TadA-8a | C | | S | R | N | N | D | Y | I | N |
| | TadA-8b | | A | S | R | N | N | | Y | I | N |
| | TadA-8c | C | | S | R | N | N | | Y | I | N |
| | TadA-8d | | A | | R | N | | | Y | | |
| | TadA-8e | | | S | R | N | N | D | Y | I | N |

In some embodiments, the TadA variant is a variant as shown in Table 5E. Table 5E shows certain amino acid position numbers in the TadA amino acid sequence and the amino acids present in those positions in the TadA*7.10 adenosine deaminase. In some embodiments, the TadA variant is MSP605, MSP680, MSP823, MSP824, MSP825, MSP827, MSP828, or MSP829. In some embodiments, the TadA variant is MSP828. In some embodiments, the TadA variant is MSP829.

TABLE 5E
TadA Variants

| | TadA Amino Acid Number | | | | | | | |
|---|---|---|---|---|---|---|---|---|
| Variant | 36 | 76 | 82 | 147 | 149 | 154 | 157 | 167 |
| TadA-7.10 | L | I | V | Y | F | Q | N | D |
| MSP605 | | | G | T | | S | | |
| MSP680 | | Y | G | T | | S | | |
| MSP823 | H | | G | T | | S | K | |
| MSP824 | | | G | D | Y | S | | N |
| MSP825 | H | | G | D | Y | S | K | N |
| MSP827 | H | Y | G | T | | S | K | |
| MSP828 | | Y | G | D | Y | S | | N |
| MSP829 | H | Y | G | D | Y | S | K | N |

In particular embodiments, the fusion proteins or complexes comprise a single (e.g., provided as a monomer) TadA*(e.g., TadA*8 or TadA*9). Throughout the present disclosure, an adenosine deaminase base editor that comprises a single TadA*domain is indicates using the terminology ABEm or ABE #m, where “#” is an identifying number (e.g., ABE8.20m), where “m” indicates “monomer.” In some embodiments, the TadA*is linked to a Cas9 nickase. In some embodiments, the fusion proteins or complexes of the disclosure comprise as a heterodimer of a wild-type TadA (TadA (wt)) linked to a TadA*. Throughout the present disclosure, an adenosine deaminase base editor that comprises a single TadA* domain and a TadA (wt) domain is indicates using the terminology ABEd or ABE #d, where “#” is an identifying number (e.g., ABE8.20d), where “d” indicates “dimer.” In other embodiments, the fusion proteins or complexes of the disclosure comprise as a heterodimer of a TadA*7.10 linked to a TadA*. In some embodiments, the base editor is ABE8 comprising a TadA*variant monomer. In some embodiments, the base editor is ABE comprising a heterodimer of a TadA*and a TadA (wt). In some embodiments, the base editor is ABE comprising a heterodimer of a TadA*and TadA*7.10. In some embodiments, the base editor is ABE comprising a heterodimer of a TadA*. In some embodiments, the TadA* is selected from Tables 5A-5E.

In some embodiments, the adenosine deaminase is expressed as a monomer. In other embodiments, the adenosine deaminase is expressed as a heterodimer. In some embodiments, the deaminase or other polypeptide sequence lacks a methionine, for example when included as a component of a fusion protein. This can alter the numbering of positions. However, the skilled person will understand that such corresponding mutations refer to the same mutation.

Any of the mutations provided herein and any additional mutations (e.g., based on the ecTadA amino acid sequence) can be introduced into any other adenosine deaminases. Any of the mutations provided herein can be made individually or in any combination in a TadA reference sequence or another adenosine deaminase (e.g., ecTadA).

Details of A to G nucleobase editing proteins are described in International PCT Application No. PCT/US2017/045381 (WO2018/027078) and Gaudelli, N.M., et al., “Programmable base editing of A.T to G.C in genomic DNA without DNA cleavage” Nature, 551, 464-471 (2017), the entire contents of which are hereby incorporated by reference.

### C to T Editing

In some embodiments, a base editor disclosed herein comprises a fusion protein or complex comprising cytidine deaminase capable of deaminating a target cytidine (C) base of a polynucleotide to produce uridine (U), which has the base pairing properties of thymine. In some embodiments, for example where the polynucleotide is double-stranded (e.g., DNA), the uridine base can then be substituted with a thymidine base (e.g., by cellular repair machinery) to give rise to a C: G to a T: A transition. In other embodiments, deamination of a C to U in a nucleic acid by a base editor cannot be accompanied by substitution of the U to a T.

The deamination of a target C in a polynucleotide to give rise to a U is a non-limiting example of a type of base editing that can be executed by a base editor described herein. In another example, a base editor comprising a cytidine deaminase domain can mediate conversion of a cytosine (C) base to a guanine (G) base. For example, a U of a polynucleotide produced by deamination of a cytidine by a cytidine deaminase domain of a base editor can be excised from the polynucleotide by a base excision repair mechanism (e.g., by a uracil DNA glycosylase (UDG) domain), producing an abasic site. The nucleobase opposite the abasic site can then be substituted (e.g., by base repair machinery) with another base, such as a C, by for example a translesion polymerase. Although it is typical for a nucleobase opposite an abasic site to be replaced with a C, other substitutions (e.g., A, G or T) can also occur.

Accordingly, in some embodiments a base editor described herein comprises a deamination domain (e.g., cytidine deaminase domain) capable of deaminating a target C to a U in a polynucleotide. Further, as described below, the base editor can comprise additional domains which facilitate conversion of the U resulting from deamination to, in some embodiments, a T or a G. For example, a base editor comprising a cytidine deaminase domain can further comprise a uracil glycosylase inhibitor (UGI) domain to mediate substitution of a U by a T, completing a C-to-T base editing event. In another example, the base editor can comprise a uracil stabilizing protein as described herein. In another example, a base editor can incorporate a translesion polymerase to improve the efficiency of C-to-G base editing, since a translesion polymerase can facilitate incorporation of a C opposite an abasic site (i.e., resulting in incorporation of a G at the abasic site, completing the C-to-G base editing event).

A base editor comprising a cytidine deaminase as a domain can deaminate a target C in any polynucleotide, including DNA, RNA and DNA-RNA hybrids.

In some embodiments, a cytidine deaminase of a base editor comprises all or a portion (e.g., a functional portion) of an apolipoprotein B mRNA editing complex (APOBEC) family deaminase. APOBEC is a family of evolutionarily conserved cytidine deaminases. Members of this family are C-to-U editing enzymes. The N-terminal domain of APOBEC like proteins is the catalytic domain, while the C-terminal domain is a pseudocatalytic domain. More specifically, the catalytic domain is a zinc dependent cytidine deaminase domain and is important for cytidine deamination. APOBEC family members include APOBEC1, APOBEC2, APOBEC3A, APOBEC3B, APOBEC3C, APOBEC3D (“APOBEC3E” now refers to this), APOBEC3F, APOBEC3G, APOBEC3H, APOBEC4, and Activation-induced (cytidine) deaminase.

Other exemplary deaminases that can be fused to Cas9 according to aspects of this disclosure are provided below. In embodiments, the deaminases are activation-induced deaminases (AID). It should be understood that, in some embodiments, the active domain of the respective sequence can be used, e.g., the domain without a localizing signal (nuclear localization sequence, without nuclear export signal, cytoplasmic localizing signal).

Some aspects of the present disclosure are based on the recognition that modulating the deaminase domain catalytic activity of any of the fusion proteins or complexes described herein, for example by making point mutations in the deaminase domain, affect the processivity of the fusion proteins (e.g., base editors) or complexes. For example, mutations that reduce, but do not eliminate, the catalytic activity of a deaminase domain within a base editing fusion protein or complexes can make it less likely that the deaminase domain will catalyze the deamination of a residue adjacent to a target residue, thereby narrowing the deamination window. The ability to narrow the deamination window can prevent unwanted deamination of residues adjacent to specific target residues, which can reduce or prevent off-target effects.

In some embodiments, an APOBEC deaminase incorporated into a base editor can comprise one or more mutations selected from the group consisting of H121R, H122R, R126A, R126E, R118A, W90A, W90Y, and R132E of rAPOBEC1; D316R, D317R, R320A, R320E, R313A, W285A, W285Y, and R326E of hAPOBEC3G; and any alternative mutation at the corresponding position, or one or more corresponding mutations in another APOBEC deaminase.

A number of modified cytidine deaminases are commercially available, including, but not limited to, SaBE3, SaKKH-BE3, VQR-BE3, EQR-BE3, VRER-BE3, YE1-BE3, EE-BE3, YE2-BE3, and YEE-BE3, which are available from Addgene (plasmids 85169, 85170, 85171, 85172, 85173, 85174, 85175, 85176, 85177). In some embodiments, a deaminase incorporated into a base editor comprises all or a portion (e.g., a functional portion) of an APOBEC1 deaminase.

In some embodiments, the fusion proteins or complexes of the disclosure comprise one or more cytidine deaminase domains. In some embodiments, the cytidine deaminases provided herein are capable of deaminating cytosine or 5-methylcytosine to uracil or thymine. In some embodiments, the cytidine deaminases provided herein are capable of deaminating cytosine in DNA. The cytidine deaminase may be derived from any suitable organism. In some embodiments, the cytidine deaminase is a naturally-occurring cytidine deaminase that includes one or more mutations corresponding to any of the mutations provided herein. One of skill in the art will be able to identify the corresponding residue in any homologous protein, e.g., by sequence alignment and determination of homologous residues. Accordingly, one of skill in the art would be able to generate mutations in any naturally-occurring cytidine deaminase that corresponds to any of the mutations described herein. In some embodiments, the cytidine deaminase is from a prokaryote. In some embodiments, the cytidine deaminase is from a bacterium. In some embodiments, the cytidine deaminase is from a mammal (e.g., human).

In some embodiments, the cytidine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the cytidine deaminase amino acid sequences set forth herein. It should be appreciated that cytidine deaminases provided herein may include one or more mutations (e.g., any of the mutations provided herein). Some embodiments provide a polynucleotide molecule encoding the cytidine deaminase nucleobase editor polypeptide of any previous aspect or as delineated herein. In some embodiments, the polynucleotide is codon optimized.

In embodiments, a fusion protein of the disclosure comprises two or more nucleic acid editing domains.

Details of C to T nucleobase editing proteins are described in International PCT Application No. PCT/US2016/058344 (WO2017/070632) and Komor, A. C., et al., “Programmable editing of a target base in genomic DNA without double-stranded DNA cleavage” Nature 533, 420-424 (2016), the entire contents of which are hereby incorporated by reference.

### Cytidine Adenosine Base Editors (CABEs)

In some embodiments, a base editor described herein comprises an adenosine deaminase variant that has increased cytidine deaminase activity. Such base editors may be referred to as “cytidine adenosine base editors (CABEs)” or “cytosine base editors derived from TadA*(CBE-Ts),” and their corresponding deaminase domains may be referred to as “TadA*acting on DNA cytosine (TADC)” domains. In some instances, an adenosine deaminase variant has both adenine and cytosine deaminase activity (i.e., is a dual deaminase). In some embodiments, the adenosine deaminase variants deaminate adenine and cytosine in DNA. In some embodiments, the adenosine deaminase variants deaminate adenine and cytosine in single-stranded DNA. In some embodiments, the adenosine deaminase variants deaminate adenine and cytosine in RNA. In some embodiments, the adenosine deaminase variant predominantly deaminates cytosine in DNA and/or RNA (e.g., greater than 30%, 40%, 50%, 60%, 70%, 80%, 90%, 95%, or 99% of all deaminations catalyzed by the adenosine deaminase variant, or the number of cytosine deaminations catalyzed by the variant is about or at least about 2-fold, 3-fold, 4-fold, 5-fold, 6-fold, 7-fold, 8-fold, 9-fold, 10-fold, 25-fold, 50-fold, 75-fold, 100-fold, 500-fold, or 1,000-fold greater than the number adenine deaminations catalyzed by the variant). In some embodiments, the adenosine deaminase variant has approximately equal cytosine and adenosine deaminase activity (e.g., the two activities are within about 10% or 20% of each other). In some embodiments, the adenosine deaminase variant has predominantly cytosine deaminase activity, and little, if any, adenosine deaminase activity. In some embodiments, the adenosine deaminase variant has cytosine deaminase activity, and no significant or no detectable adenosine deaminase activity. In some embodiments, the target polynucleotide is present in a cell in vitro or in vivo. In some embodiments, the cell is a bacteria, yeast, fungi, insect, plant, or mammalian cell.

In some embodiments, the CABE comprises a bacterial TadA deaminase variant (e.g., ecTadA). In some embodiments, the CABE comprises a truncated TadA deaminase variant. In some embodiments, the CABE comprises a fragment of a TadA deaminase variant. In some embodiments, the CABE comprises a TadA*8.20 variant.

In some embodiments, an adenosine deaminase variant of the disclosure is a TadA adenosine deaminase comprising one or more alterations that increase cytosine deaminase activity (e.g., at least about 10-fold, 20-fold, 30-fold, 40-fold, 50-fold, 60-fold, 70-fold or more increase) while maintaining adenosine deaminase activity (e.g., at least about 30%, 40%, 50% or more of the activity of a reference adenosine deaminase (e.g., TadA*8.20 or TadA*8.19)). In some instances, the adenosine deaminase variant comprises one or more alterations that increase cytosine deaminase activity (e.g., at least about 10-fold, 20-fold, 30-fold, 40-fold, 50-fold, 60-fold, 70-fold or more increase) relative to the activity of a reference adenosine deaminase and comprise undetectable adenosine deaminase activity or adenosine deaminase activity that is less than 30%, 20%, 10%, or 5% of that of a reference adenosine deaminase. In some embodiments, the reference adenosine deaminase is TadA*8.20 or TadA*8.19.

In some embodiments, the adenosine deaminase variant is an adenosine deaminase comprising two or more alterations at an amino acid position selected from the group consisting of 2, 4, 6, 8, 13, 17, 23, 27, 29, 30, 47, 48, 49, 67, 76, 77, 82, 84, 96, 100, 107, 112, 114, 115, 118, 119, 122, 127, 142, 143, 147, 149, 158, 159, 162 165, 166, and 167, of an amino acid sequence having at least about 70%, 75%, 80%, 85%, 90%, 95%, 98%, 99% or greater identity to SEQ ID NO: 1, or a corresponding alteration in another deaminase. I

In some embodiments, the adenosine deaminase variant is an adenosine deaminase comprising one or more alterations selected from the group consisting of S2H, V4K, V4S, V4T, V4Y, F6G, F6H, F6Y, H8Q, R13G, T17A, T17W, R23Q, E27C, E27G, E27H, E27K, E27Q, E27S, E27G, P29A, P29G, P29K, V30F, V30I, R47G, R47S, A48G, I49K, I49M, I49N, 149Q, 149T, G67W, 176H, I76R, I76W, Y76H, Y76R, Y76W, F84A, F84M, H96N, G100A, G100K, T111H, G112H, A114C, G115M, M118L, H122G, H122R, H122T, N127I, N127K, N127P, A142E, R147H, A158V, Q159S, A162C, A162N, A162Q, and S165P of an amino acid sequence having at least about 70%, 75%, 80%, 85%, 90%, 95%, 98%, 99% or greater identity to SEQ ID NO: 1, or a corresponding alteration in another deaminase.

In some embodiments, the adenosine deaminase variant is an adenosine deaminase comprising an amino acid alteration or combination of amino acid alterations selected from those listed in any of Tables 6A-6F.

The residue identity of exemplary adenosine deaminase variants that are capable of deaminating adenine and/or cytidine in a target polynucleotide (e.g., DNA) is provided in Tables 6A-6F below. Further examples of adenosine deaminase variants include the following variants of 1.17 (see Table 6A): 1.17+E27H; 1.17+E27K; 1.17+E27S; 1.17+E27S+I49K; 1.17+E27G; 1.17+149N; 1.17+E27G+149N; and 1.17+E27Q. In some embodiments, any of the amino acid alterations provided herein are substituted with a conservative amino acid. Additional mutations known in the art can be further added to any of the adenosine deaminase variants provided herein.

In some embodiments, the base editor systems comprising a CABE provided herein have at least about a 30%, 40%, 50%, 60%, 70% or more C to T editing activity in a target polynucleotide (e.g., DNA). In some embodiments, a base editor system comprising a CABE as provided herein has an increased C to T base editing activity (e.g., increased at least about 30-fold, 40-fold, 50-fold, 60-fold, 70-fold or more) relative to a reference base editor system comprising a reference adenosine deaminase (e.g., TadA*8.20 or TadA*8.19).

TABLE 6A
Adenosine Deaminase Variants. Mutations are indicated with reference to
TadA*8.20. “S” indicates “Surface,” and “NAS” indicates “Near Active Site.”

| location in | | | | | | | | | | | |
|---|---|---|---|---|---|---|---|---|---|---|---|
| structure | N/A | S h1 | S h1 | S h1 | NAS | NAS | NAS | NAS | | S | |
| Amino Acid | | | | | | | | | | | |
| No. (*START | | | | | | | | | | | |
| Met is AA#1) | 2 | 8 | 13 | 17 | 27 | 47 | 48 | 49 | 67 | 76 | 77 |
| TadA*8.20 | S | H | R | T | E | R | A | I | G | Y | D |
| TadA*8.19 | | | | | | | | | | I | |
| 1.1 | | | | | H | | | | | I | |
| 1.2 | | | | | H | | | K | | I | |
| 1.3 | | | | | S | | | K | | I | |
| 1.4 | | | | | S | | | K | | I | |
| 1.5 | | | | | K | | | | | | |
| 1.6 | | | | | K | | | | | | |
| 1.7 | | | | | H | | | | | I | |
| 1.8 | | | | | S | | | K | W | | |
| 1.9 | | | | | | | | T | W | | |
| 1.10 | | | | | C | | | | | I | |
| 1.11 | | | G | | Q | | | | | | |
| 1.12 | | | | A | H | | | M | | I | |
| 1.13 | | | | | | | | Q | | I | |
| 1.14 | H | | | | | | | K | | I | |
| 1.15 | | | | | | S | | | | | |
| 1.16 | | Q | | | | | | Q | | I | |
| 1.17 | | | | A | | | G | | | | |
| 1.18 | | | | | G | | | | | | |
| 1.19 | | | | | G | | | N | | | |
| 1.20 | | | | | G | | | | | | G |

Adenosine Deaminase Variants. Mutations are indicated with reference to
TadA*8.20. “I” indicates “Internal,” “S” indicates “Surface,” and “NAS” indicates “Near Active Site.”

| location in | | | | | | | | | | | | |
|---|---|---|---|---|---|---|---|---|---|---|---|---|
| structure | I | NAS | | NAS | S | | S | S | S | | S | |
| Amino Acid | | | | | | | | | | | | |
| No. (*START | | | | | | | | | | | | |
| Met is AA#1) | 82 | 84 | 96 | 107 | 112 | 115 | 118 | 119 | 127 | 142 | 162 | 165 |
| TadA*8.20 | S | F | H | R | G | G | M | D | N | A | A | S |
| TadA*8.19 | | | | | | | | | | | | |
| 1.1 | | M | | | | | | | | | | |
| 1.2 | | | | | | | | | | | | |
| 1.3 | | | | | | | | | | | | |
| 1.4 | | | | | | | | | | | N | |
| 1.5 | | | | | | | | | | | | |
| 1.6 | | | | | | | | N | | | | |
| 1.7 | | | | | | | | | | | | |
| 1.8 | | | | | | | | | | | | |
| 1.9 | | | N | | | | | | | | | |
| 1.10 | | | | | | | | N | | | | |
| 1.11 | | | | | | | | | K | | | |
| 1.12 | | | | | | | L | | | | | |
| 1.13 | | | | | | M | | | | | | |
| 1.14 | | | | | H | | | | | | | |
| 1.15 | | | | C | | | | | | | | |
| 1.16 | | | | | | | | | | | | |
| 1.17 | T | | | | | | | | | E | | |
| 1.18 | | | | | | | | | | | | |
| 1.19 | | | | | | | | | | | | |
| 1.20 | | | | | | | | | | | | P |

TABLE 6B
Adenosine deaminase variants. Mutations are indicated
with reference to TadA*8.20.

| Position | | | | | | | | | | |
|---|---|---|---|---|---|---|---|---|---|---|
| No. | 27 | 29 | 30 | 49 | 82 | 84 | 107 | 112 | 115 | 142 |
| TadA*8.20 | E | P | V | I | S | F | R | G | G | A |
| Alterations | | | | | | | | | | |
| Evaluated | G/S/H | G/A/K | I/L/F | K | T | L/A | C | H | M | E |
| S1.1 | S | | | K | T | | | | | |
| S1.2 | S | | | K | T | | C | | | |
| S1.3 | S | | | K | T | | | H | | |
| S1.4 | S | | | K | T | | | | M | |
| S1.5 | S | | | K | T | | | | | E |
| S1.6 | S | | | K | T | | C | H | | |
| S1.7 | S | | | K | T | | C | | M | |
| S1.8 | S | | | K | T | | C | | | E |
| S1.9 | S | | | K | T | | | H | | E |
| S1.10 | S | | | K | T | | | | M | E |
| S1.11 | S | | | K | T | | C | H | M | E |
| S1.12 | S | | I | K | T | | | | | |
| S1.13 | S | | I | K | T | | C | | | |
| S1.14 | S | | I | K | T | | | H | | |
| S1.15 | S | | I | K | T | | | | M | |
| S1.16 | S | | I | K | T | | | | | E |
| S1.17 | S | | I | K | T | | C | H | | |
| S1.18 | S | | I | K | T | | C | | M | |
| S1.19 | S | | I | K | T | | C | | | E |
| S1.20 | S | | I | K | T | | | H | | E |
| S1.21 | S | | I | K | T | | | | M | E |
| S1.22 | S | | I | K | T | | C | H | M | E |
| S1.23 | S | | L | K | T | | | | | |
| S1.24 | S | | L | K | T | | C | | | |
| S1.25 | S | | L | K | T | | | H | | |
| S1.26 | S | | L | K | T | | | | M | |
| S1.27 | S | | L | K | T | | | | | E |
| S1.28 | S | | L | K | T | | C | H | | |
| S1.29 | S | | L | K | T | | C | | M | |
| S1.30 | S | | L | K | T | | C | | | E |
| S1.31 | S | | L | K | T | | | H | | E |
| S1.32 | S | | L | K | T | | | | M | E |
| S1.33 | S | | L | K | T | | C | H | M | E |
| S1.34 | S | | F | K | T | A | | | | |
| S1.35 | S | | F | K | T | A | C | | | |
| S1.36 | S | | F | K | T | A | | H | | |
| S1.37 | S | | F | K | T | A | | | M | |
| S1.38 | S | | F | K | T | A | | | | E |
| S1.39 | S | | F | K | T | A | C | H | | |
| S1.40 | S | | F | K | T | A | C | | M | |
| S1.41 | S | | F | K | T | A | C | | | E |
| S1.42 | S | | F | K | T | A | | H | | E |
| S1.43 | S | | F | K | T | A | | | M | E |
| S1.44 | S | | F | K | T | A | C | H | M | E |
| S1.45 | S | | | K | T | L | | | | |
| S1.46 | S | | | K | T | L | C | | | |
| S1.47 | S | | | K | T | L | | H | | |
| S1.48 | S | | | K | T | L | | | M | |
| S1.49 | S | | | K | T | L | | | | E |
| S1.50 | S | | | K | T | L | C | H | | |
| S1.51 | S | | | K | T | L | C | | M | |
| S1.52 | S | | | K | T | L | C | | | E |
| S1.53 | S | | | K | T | L | | H | | E |
| S1.54 | S | | | K | T | L | | | M | E |
| S1.55 | S | | | K | T | L | C | H | M | E |
| S1.56 | S | | I | K | T | L | | | | |
| S1.57 | S | | I | K | T | L | C | | | |
| S1.58 | S | | I | K | T | L | | H | | |
| S1.59 | S | | I | K | T | L | | | M | |
| S1.60 | S | | I | K | T | L | | | | E |
| S1.61 | S | | I | K | T | L | C | H | | |
| S1.62 | S | | I | K | T | L | C | | M | |
| S1.63 | S | | I | K | T | L | C | | | E |
| S1.64 | S | | I | K | T | L | | H | | E |
| S1.65 | S | | I | K | T | L | | | M | E |
| S1.66 | S | | I | K | T | L | C | H | M | E |
| S1.67 | S | G | | K | T | | | | | |
| S1.68 | S | G | | K | T | | C | | | |
| S1.69 | S | G | | K | T | | | H | | |
| S1.70 | S | G | | K | T | | | | M | |
| S1.71 | S | G | | K | T | | | | | E |
| S1.72 | S | G | | K | T | | C | H | | |
| S1.73 | S | G | | K | T | | C | | M | |
| S1.74 | S | G | | K | T | | C | | | E |
| S1.75 | S | G | | K | T | | | H | | E |
| S1.76 | S | G | | K | T | | | | M | E |
| S1.77 | S | G | | K | T | | C | H | M | E |
| S1.78 | | G | | K | T | | | | | |
| S1.79 | | G | | K | T | | C | | | |
| S1.80 | | G | | K | T | | | H | | |
| S1.81 | | G | | K | T | | | | M | |
| S1.82 | | G | | K | T | | | | | E |
| S1.83 | | G | | K | T | | C | H | | |
| S1.84 | | G | | K | T | | C | | M | |
| S1.85 | | G | | K | T | | C | | | E |
| S1.86 | | G | | K | T | | | H | | E |
| S1.87 | | G | | K | T | | | | M | E |
| S1.88 | | G | | K | T | | C | H | M | E |
| S1.89 | | K | | K | T | | | | | |
| S1.90 | | K | | K | T | | C | | | |
| S1.91 | | K | | K | T | | | H | | |
| S1.92 | | K | | K | T | | | | M | |
| S1.93 | | K | | K | T | | | | | E |
| S1.94 | | K | | K | T | | C | H | | |
| S1.95 | | K | | K | T | | C | | M | |
| S1.96 | | K | | K | T | | C | | | E |
| S1.97 | | K | | K | T | | | H | | E |
| S1.98 | | K | | K | T | | | | M | E |
| S1.99 | | K | | K | T | | C | H | M | E |
| S1.100 | | K | I | K | T | | | | | |
| S1.101 | | K | I | K | T | | C | | | |
| S1.102 | | K | I | K | T | | | H | | |
| S1.103 | | K | I | K | T | | | | M | |
| S1.104 | | K | I | K | T | | | | | E |
| S1.105 | | K | I | K | T | | C | H | | |
| S1.106 | | K | I | K | T | | C | | M | |
| S1.107 | | K | I | K | T | | C | | | E |
| S1.108 | | K | I | K | T | | | H | | E |
| S1.109 | | K | I | K | T | | | | M | E |
| S1.110 | | K | I | K | T | | C | H | M | E |
| S1.111 | | K | | K | T | L | | | | |
| S1.112 | | K | | K | T | L | C | | | |
| S1.113 | | K | | K | T | L | | H | | |
| S1.114 | | K | | K | T | L | | | M | |
| S1.115 | | K | | K | T | L | | | | E |
| S1.116 | | K | | K | T | L | C | H | | |
| S1.117 | | K | | K | T | L | C | | M | |
| S1.118 | | K | | K | T | L | C | | | E |
| S1.119 | | K | | K | T | L | | H | | E |
| S1.120 | | K | | K | T | L | | | M | E |
| S1.121 | | K | | K | T | L | C | H | M | E |
| S1.122 | | K | I | K | T | L | | | | |
| S1.123 | | K | I | K | T | L | C | | | |
| S1.124 | | K | I | K | T | L | | H | | |
| S1.125 | | K | I | K | T | L | | | M | |
| S1.126 | | K | I | K | T | L | | | | E |
| S1.127 | | K | I | K | T | L | C | H | | |
| S1.128 | | K | I | K | T | L | C | | M | |
| S1.129 | | K | I | K | T | L | C | | | E |
| S1.130 | | K | I | K | T | L | | H | | E |
| S1.131 | | K | I | K | T | L | | | M | E |
| S1.132 | | K | I | K | T | L | C | H | M | E |
| S1.133 | G | | | K | T | | | | | |
| S1.134 | G | | | K | T | | C | | | |
| S1.135 | G | | | K | T | | | H | | |
| S1.136 | G | | | K | T | | | | M | |
| S1.137 | G | | | K | T | | | | | E |
| S1.138 | G | | | K | T | | C | H | | |
| S1.139 | G | | | K | T | | C | | M | |
| S1.140 | G | | | K | T | | C | | | E |
| S1.141 | G | | | K | T | | | H | | E |
| S1.142 | G | | | K | T | | | | M | E |
| S1.143 | G | | | K | T | | C | H | M | E |
| S1.144 | H | | | K | T | | | | | |
| S1.145 | H | | | K | T | | C | | | |
| S1.146 | H | | | K | T | | | H | | |
| S1.147 | H | | | K | T | | | | M | |
| S1.148 | H | | | K | T | | | | | E |
| S1.149 | H | | | K | T | | C | H | | |
| S1.150 | H | | | K | T | | C | | M | |
| S1.151 | H | | | K | T | | C | | | E |
| S1.152 | H | | | K | T | | | H | | E |
| S1.153 | H | | | K | T | | | | M | E |
| S1.154 | H | | | K | T | | C | H | M | E |
| S1.155 | S | | | | T | | | | | |
| S1.156 | S | | | | T | | C | | | |
| S1.157 | S | | | | T | | | H | | |
| S1.158 | S | | | | T | | | | M | |
| S1.159 | S | | | | T | | | | | E |
| S1.160 | S | | | | T | | C | H | | |
| S1.161 | S | | | | T | | C | | M | |
| S1.162 | S | | | | T | | C | | | E |
| S1.163 | S | | | | T | | | H | | E |
| S1.164 | S | | | | T | | | | M | E |
| S1.165 | S | | | | T | | C | H | M | E |
| S1.166 | | A | | | T | | | | | |
| S1.167 | | A | | | T | | C | | | |
| S1.168 | | A | | | T | | | H | | |
| S1.169 | | A | | | T | | | | M | |
| S1.170 | | A | | | T | | | | | E |
| S1.171 | | A | | | T | | C | H | | |
| S1.172 | | A | | | T | | C | | M | |
| S1.173 | | A | | | T | | C | | | E |
| S1.174 | | A | | | T | | | H | | E |
| S1.175 | | A | | | T | | | | M | E |
| S1.176 | | A | | | T | | C | H | M | E |
| S1.177 | S | | I | | T | | | | | |
| S1.178 | S | | I | | T | | C | | | |
| S1.179 | S | | I | | T | | | H | | |
| S1.180 | S | | I | | T | | | | M | |
| S1.181 | S | | I | | T | | | | | E |
| S1.182 | S | | I | | T | | C | H | | |
| S1.183 | S | | I | | T | | C | | M | |
| S1.184 | S | | I | | T | | C | | | E |
| S1.185 | S | | I | | T | | | H | | E |
| S1.186 | S | | I | | T | | | | M | E |
| S1.187 | S | | I | | T | | C | H | M | E |
| S1.188 | | A | I | | T | L | | | | |
| S1.189 | | A | I | | T | L | C | | | |
| S1.190 | | A | I | | T | L | | H | | |
| S1.191 | | A | I | | T | L | | | M | |
| S1.192 | | A | I | | T | L | | | | E |
| S1.193 | | A | I | | T | L | C | H | | |
| S1.194 | | A | I | | T | L | C | | M | |
| S1.195 | | A | I | | T | L | C | | | E |
| S1.196 | | A | I | | T | L | | H | | E |
| S1.197 | | A | I | | T | L | | | M | E |
| S1.198 | | A | I | | T | L | C | H | M | E |
| S1.199 | S | A | L | K | T | L | C | H | M | E |

TABLE 6C
Adenosine deaminase variants. Mutations are indicated with reference to variant 1.2 (Table 6A).

| | | Residue identity (START Met is amino | | | | | | | | |
|---|---|---|---|---|---|---|---|---|---|---|
| | | acid #1) | | | | | | | | |
| Variant Name | Alternative Variant Names | 4 | 6 | 17 | 23 | 76 | 77 | 100 | 111 | 114 |
| Reference | 1.2 (see Table 6A) | V | F | T | R | I | D | G | T | A |
| TadAC2.1 | pDKL-135; 2.1 | K | | | | | | | | C |
| TadAC2.2 | pDKL-136; 2.2 | K | | | | | G | | | |
| TadAC2.3 | pDKL-137; 2.3 | | Y | | | | | A | | |
| TadAC2.4 | pDKL-138; 2.4 | T | | | | R | | | | |
| TadAC2.5 | pDKL-139; 2.5 | | Y | | | W | | | | |
| TadAC2.6 | pDKL-140; 2.6 | | Y | | | | | | | |
| TadAC2.7 | pDKL-141; 2.7 | | Y | | | | | | | C |
| TadAC2.8 | pDKL-142; 2.8 | | Y | | | | | | | |
| TadAC2.9 | pDKL-143; 2.9 | K | | | | W | | | | |
| TadAC2.10 | pDKL-144; 2.10 | | G | | | R | | K | | |
| TadAC2.11 | pDKL-145; 2.11 | | H | | | | | | | |
| TadAC2.12 | pDKL-146; 2.12 | | | | | | | | | C |
| TadAC2.13 | pDKL-147; 2.13 | | Y | | | H | | | | |
| TadAC2.14 | pDKL-148; 2.14 | | | | | | | | | |
| TadAC2.15 | pDKL-149; 2.15 | | | | Q | R | | | | |
| TadAC2.16 | pDKL-150; 2.16 | | | | | H | | | | |
| TadAC2.17 | pDKL-151; 2.17 | | Y | | | | | | H | |
| TadAC2.18 | pDKL-152; 2.18 | | | | | W | | | | |
| TadAC2.19 | pDKL-153; 2.19 | | | | | | | | H | |
| TadAC2.20 | pDKL-154; 2.20 | | | | | | | | | |
| TadAC2.21 | pDKL-155; 2.21 | | Y | | | R | | | | |
| TadAC2.22 | pDKL-156; 2.22 | | | W | | H | | | | |
| TadAC2.23 | pDKL-157; 2.23 | S | | | | Y | | | | |
| TadAC2.24 | pDKL-158; 2.24 | | | | | | | | | |
| | | Residue identity (START Met is | | | | | | | | |
| | | amino acid #1) | | | | | | | | |
| Variant Name | Alternative Variant Names | 119 | 122 | 127 | 143 | 147 | 158 | 159 | 162 | 166 |
| Reference | 1.2 (see Table 6A) | D | H | N | A | R | A | Q | A | T |
| TadAC2.1 | pDKL-135; 2.1 | | | | | | | | | |
| TadAC2.2 | pDKL-136; 2.2 | | | | | | | | | |
| TadAC2.3 | pDKL-137; 2.3 | | R | | | | | | | |
| TadAC2.4 | pDKL-138; 2.4 | | G | | | | | | | |
| TadAC2.5 | pDKL-139; 2.5 | | | | | | | | | |
| TadAC2.6 | pDKL-140; 2.6 | N | | | | | | | | |
| TadAC2.7 | pDKL-141; 2.7 | | | | | | | | | |
| TadAC2.8 | pDKL-142; 2.8 | | | | | | | | | |
| TadAC2.9 | pDKL-143; 2.9 | | T | | | | | | | |
| TadAC2.10 | pDKL-144; 2.10 | | | | | | | | | |
| TadAC2.11 | pDKL-145; 2.11 | | N | | | | | | | |
| TadAC2.12 | pDKL-146; 2.12 | | | | | | | | | |
| TadAC2.13 | pDKL-147; 2.13 | | R | | | | | | | I |
| TadAC2.14 | pDKL-148; 2.14 | | | P | | | | | | |
| TadAC2.15 | pDKL-149; 2.15 | | | | | | | | | |
| TadAC2.16 | pDKL-150; 2.16 | | R | | | | V | | | |
| TadAC2.17 | pDKL-151; 2.17 | | | | | | | | | |
| TadAC2.18 | pDKL-152; 2.18 | | | | | | | | | |
| TadAC2.19 | pDKL-153; 2.19 | | G | | | | | | C | |
| TadAC2.20 | pDKL-154; 2.20 | | | | E | | | | | |
| TadAC2.21 | pDKL-155; 2.21 | | | | | | | | | |
| TadAC2.22 | pDKL-156; 2.22 | | G | | | | V | | | |
| TadAC2.23 | pDKL-157; 2.23 | | | | E | | | S | | |
| TadAC2.24 | pDKL-158; 2.24 | | | I | | | | | Q | |

TABLE 6D
Adenosine deaminase variants. Mutations are indicated with reference to TadA*8.20.

| AA Positions | 6 | 27 | 49 | 76 | 77 | 82 | 107 | 112 | 114 | 115 | 119 | 122 | 127 | 142 | 143 |
|---|---|---|---|---|---|---|---|---|---|---|---|---|---|---|---|
| TadA*8.20 | F | E | I | Y | D | S | R | G | A | G | D | H | N | A | A |
| S1.154 | F | H | K | Y | D | T | C | H | | M | | | | E | |
| Alterations | Y | | | W | G | | | | C | | N | G | P | | E |
| from Table | | | | | | | | | | | | | | | |
| 6C | | | | | | | | | | | | | | | |
| S2.1 | Y | H | K | W | | T | C | H | | M | | | | E | |
| S2.2 | Y | H | K | | G | T | C | H | | M | | | | E | |
| S2.3 | Y | H | K | | | T | C | H | C | M | | | | E | |
| S2.4 | Y | H | K | | | T | C | H | | M | N | | | E | |
| S2.5 | Y | H | K | | | T | C | H | | M | | G | | E | |
| S2.6 | Y | H | K | | | T | C | H | | M | | | P | E | |
| S2.7 | Y | H | K | | | T | C | H | | M | | | | E | E |
| S2.8 | Y | H | K | | | T | C | H | | M | | | | A | E |
| S2.9 | Y | H | K | W | G | T | C | H | | M | | | | E | |
| S2.10 | Y | H | K | W | | T | C | H | C | M | | | | E | |
| S2.11 | Y | H | K | W | | T | C | H | | M | N | | | E | |
| S2.12 | Y | H | K | W | | T | C | H | | M | | G | | E | |
| S2.13 | Y | H | K | W | | T | C | H | | M | | | P | E | |
| S2.14 | Y | H | K | W | | T | C | H | | M | | | | E | E |
| S2.15 | Y | H | K | W | | T | C | H | | M | | | | A | E |
| S2.16 | Y | H | K | | G | T | C | H | C | M | | | | E | |
| S2.17 | Y | H | K | | G | T | C | H | | M | N | | | E | |
| S2.18 | Y | H | K | | G | T | C | H | | M | | G | | E | |
| S2.19 | Y | H | K | | G | T | C | H | | M | | | P | E | |
| S2.20 | Y | H | K | | G | T | C | H | | M | | | | E | E |
| S2.21 | Y | H | K | | G | T | C | H | | M | | | | A | E |
| S2.22 | Y | H | K | | | T | C | H | C | M | N | | | E | |
| S2.23 | Y | H | K | | | T | C | H | C | M | | G | | E | |
| S2.24 | Y | H | K | | | T | C | H | C | M | | | P | E | |
| S2.25 | Y | H | K | | | T | C | H | | M | N | G | | E | |
| S2.26 | Y | H | K | | | T | C | H | | M | N | | P | E | |
| S2.27 | Y | H | K | | | T | C | H | | M | | G | P | E | |
| S2.28 | Y | H | K | W | G | T | C | H | C | M | | | | E | |
| S2.29 | Y | H | K | W | G | T | C | H | | M | N | | | E | |
| S2.30 | Y | H | K | W | G | T | C | H | | M | | G | | E | |
| S2.31 | Y | H | K | W | G | T | C | H | | M | | | P | E | |
| S2.32 | Y | H | K | W | G | T | C | H | | M | | | | E | E |
| S2.33 | Y | H | K | W | G | T | C | H | | M | | | | A | E |
| S2.34 | Y | H | K | W | | T | C | H | C | M | N | | | E | |
| S2.35 | Y | H | K | W | | T | C | H | C | M | | G | | E | |
| S2.36 | Y | H | K | W | | T | C | H | C | M | | | P | E | |
| S2.37 | Y | H | K | W | | T | C | H | C | M | | | | E | E |
| S2.38 | Y | H | K | W | | T | C | H | C | M | | | | A | E |
| S2.39 | Y | H | K | W | | T | C | H | | M | N | G | | E | |
| S2.40 | Y | H | K | W | | T | C | H | | M | N | | P | E | |
| S2.41 | Y | H | K | M | | T | C | H | | M | | G | P | E | |
| S2.42 | Y | H | K | W | | T | C | H | C | M | N | G | | E | |
| S2.43 | Y | H | K | W | | T | C | H | C | M | N | | P | E | |
| S2.44 | Y | H | K | W | | T | C | H | C | M | | G | P | E | |
| S2.45 | Y | H | K | W | G | T | C | H | C | M | N | | | E | |
| S2.46 | Y | H | K | W | G | T | C | H | C | M | | G | | E | |
| S2.47 | Y | H | K | W | G | T | C | H | C | M | | | P | E | |
| S2.48 | Y | H | K | W | G | T | C | H | C | M | | | | E | E |
| S2.49 | Y | H | K | W | G | T | C | H | C | M | | | | A | E |
| S2.50 | Y | H | K | W | G | T | C | H | C | M | N | G | | E | |
| S2.51 | Y | H | K | W | G | T | C | H | C | M | N | | P | E | |
| S2.52 | Y | H | K | W | G | T | C | H | C | M | | G | P | E | |
| S2.53 | Y | H | K | W | | T | C | H | C | M | N | G | P | E | E |
| S2.54 | Y | H | K | W | | T | C | H | C | M | N | G | P | A | E |
| S2.55 | Y | H | K | W | G | T | C | H | C | M | N | G | P | E | E |
| S2.56 | Y | H | K | W | G | T | C | H | C | M | N | G | P | A | E |

TABLE 6E
Hybrid constructs. Mutations are indicated with reference
to TadA*7.10.

| | TadA amino acid subsitutions | | | | | | | | | | | |
|---|---|---|---|---|---|---|---|---|---|---|---|---|
| | 76 | 82 | 109 | 111 | 119 | 122 | 123 | 147 | 149 | 154 | 166 | 167 |
| TadA*7.10 | I | V | A | T | D | H | Y | Y | F | Q | T | D |
| TadA*8e | | | S | R | N | N | | D | Y | | I | N |
| TadA*8.20 | Y | S | | | | | H | R | | R | | |
| TadA*8.17 | | S | | | | | | | | R | | |
| pNMG-B878 | Y | S | | | | | H | D | | R | | |
| pNMG-B879 | Y | S | | | | | H | R | Y | R | | |
| pNMG-B880 | Y | S | | | | | H | R | | R | I | |
| pNMG-B881 | Y | S | | | | | H | R | | R | | N |
| pNMG-B882 | Y | S | | | | | H | D | Y | R | I | N |
| pNMG-B883 | Y | S | | R | N | | H | R | | R | | |
| pNMG-B884 | Y | S | S | R | N | N | H | R | | R | | |
| pNMG-B885 | Y | S | S | | | | H | R | | R | | |
| pNMG-B886 | Y | S | | R | | | H | R | | R | | |
| pNMG-B887 | Y | S | | | N | | H | R | | R | | |
| pNMG-B888 | Y | S | | | | N | H | R | | R | | |
| pNMG-B889 | Y | S | S | R | | | H | R | | R | | |
| pNMG-B890 | Y | S | | | N | N | H | R | | R | | |
| pNMG-B891 | Y | S | S | R | N | N | H | D | Y | R | I | N |

TABLE 6F
Base editor variants. Mutations are indicated with reference to
TadA*8.19/8.20.

| AA positions: | 17 | 27 | 48 | 49 | 76 | 82 | 84 | 118 | 142 | 147 | 149 | 166 | 167 |
|---|---|---|---|---|---|---|---|---|---|---|---|---|---|
| ABE8.19m/8.20m | T | E | A | I | Y/I | S | F | M | A | Y | F | T | D |
| 1.1 + 8e(B879) | | H | | | I | | M | | | | Y | | |
| 1.2 + 8e(B879) | | H | | K | I | | | | | | Y | | |
| 1.12 + 8e(B879) | A | H | | M | I | | | L | | | Y | | |
| 1.17 + 8e(B879) | A | | G | | | T | | | E | | Y | | |
| 1.18 + 8e(B879) | | G | | | | | | | | | Y | | |
| 1.19 + 8e(B879) | | G | | N | | | | | | | Y | | |
| 1.1 + 8e(B882) | | H | | | I | | M | | | D | Y | I | N |
| 1.2 + 8e(B882) | | H | | K | I | | | | | D | Y | I | N |
| 1.12 + 8e(B882) | A | H | | M | I | | | L | | D | Y | I | N |
| 1.17 + 8e(B882) | A | | G | | | T | | | E | D | Y | I | N |
| 1.18 + 8e(B882) | | G | | | | | | | | D | Y | I | N |
| 1.19 + 8e(B882) | | G | | N | | | | | | D | Y | I | N |

### Guide Polynucleotides

A polynucleotide programmable nucleotide binding domain, when in conjunction with a bound guide polynucleotide (e.g., gRNA), can specifically bind to a target polynucleotide sequence (i.e., via complementary base pairing between bases of the bound guide nucleic acid and bases of the target polynucleotide sequence) and thereby localize the base editor to the target nucleic acid sequence desired to be edited. In some embodiments, the target polynucleotide sequence comprises single-stranded DNA or double-stranded DNA. In some embodiments, the target polynucleotide sequence comprises RNA. In some embodiments, the target polynucleotide sequence comprises a DNA-RNA hybrid.

In an embodiment, a guide polynucleotide described herein can be RNA or DNA. In one embodiment, the guide polynucleotide is a gRNA.

In some embodiments, the guide polynucleotide is at least one single guide RNA (“sgRNA” or “gRNA”). In some embodiments, a guide polynucleotide comprises two or more individual polynucleotides, which can interact with one another via for example complementary base pairing (e.g., a dual guide polynucleotide, dual gRNA). For example, a guide polynucleotide can comprise a CRISPR RNA (crRNA) and a trans-activating CRISPR RNA (tracrRNA) or can comprise one or more trans-activating CRISPR RNA (tracrRNA).

A guide polynucleotide may include natural or non-natural (or unnatural) nucleotides (e.g., peptide nucleic acid or nucleotide analogs). In some cases, the targeting region of a guide nucleic acid sequence (e.g., a spacer) can be at least 15, 16, 17, 18, 19, 20, 21, 22, 23, 24, 25, 26, 27, 28, 29, or 30 nucleotides in length.

In some embodiments, the methods described herein can utilize an engineered Cas protein. A guide RNA (gRNA) is a short synthetic RNA composed of a scaffold sequence necessary for Cas-binding and a user-defined ˜20 nucleotide spacer that defines the genomic target to be modified. Exemplary gRNA scaffold sequences are provided in the sequence listing as SEQ ID NOs: 317-327 and 425. Thus, a skilled artisan can change the genomic target of the Cas protein specificity is partially determined by how specific the gRNA targeting sequence is for the genomic target compared to the rest of the genome. In embodiments, the spacer is about 10, 11, 12, 13, 14, 15, 16, 17, 18, 19, 20, 22, 23, 24, 25, or more nucleotides in length. The spacer of a gRNA can be or can be about 19, 20, or 21 nucleotides in length.

A gRNA or a guide polynucleotide can target any exon or intron of a gene target. In some embodiments, a composition comprises multiple gRNAs that all target the same exon or multiple gRNAs that target different exons. An exon and/or an intron of a gene can be targeted. A gRNA or a guide polynucleotide can target a nucleic acid sequence of about 20 nucleotides or less than about 20 nucleotides (e.g., at least about 5, 10, 15, 16, 17, 18, 19, 20, 21, 22, 23, 24, 25, 30 nucleotides), or anywhere between about 1-100 nucleotides (e.g., 5, 10, 15, 16, 17, 18, 19, 20, 21, 22, 23, 24, 25, 30, 40, 50, 60, 70, 80, 90, 100). A target nucleic acid sequence can be or can be about 20 bases immediately 5′ of the first nucleotide of the PAM. A gRNA can target a nucleic acid sequence. A target nucleic acid can be at least or at least about 1-10, 1-20, 1-30, 1-40, 1-50, 1-60, 1-70, 1-80, 1-90, or 1-100 nucleotides.

The guide polynucleotides can comprise standard ribonucleotides, modified ribonucleotides (e.g., pseudouridine), ribonucleotide isomers, and/or ribonucleotide analogs.

In some embodiments, a base editor system may comprise multiple guide polynucleotides, e.g., gRNAs. For example, the gRNAs may target to one or more target loci (e.g., at least 1 gRNA, at least 2 gRNA, at least 5 gRNA, at least 10 gRNA, at least 20 gRNA, at least 30 g RNA, at least 50 gRNA) comprised in a base editor system. The multiple gRNA sequences can be tandemly arranged and may be separated by a direct repeat.

### Modified Polynucleotides

To enhance expression, stability, and/or genomic/base editing efficiency, and/or reduce possible toxicity, the base editor-coding sequence (e.g., mRNA) and/or the guide polynucleotide (e.g., gRNA) can be modified to include one or more modified nucleotides and/or chemical modifications, e.g. using pseudo-uridine, 5-Methyl-cytosine, 2′-O-methyl-3′-phosphonoacetate, 2′-O-methyl thioPACE (MSP), 2′-O-methyl-PACE (MP), 2′-fluoro RNA (2′-F-RNA), =constrained ethyl (S-cEt), 2′-O-methyl (‘M’), 2′-O-methyl-3′-phosphorothioate (‘MS’), 2′-O-methyl-3′-thiophosphonoacetate (‘MSP’), 5-methoxyuridine, phosphorothioate, and N₁-Methylpseudouridine. Chemically protected gRNAs can enhance stability and editing efficiency in vivo and ex vivo. Methods for using chemically modified mRNAs and guide RNAs are known in the art and described, for example, by Jiang et al., Chemical modifications of adenine base editor mRNA and guide RNA expand its application scope. Nat Commun 11, 1979 (2020). doi.org/10.1038/s41467-020-15892-8, Callum et al., NI-Methylpseudouridine substitution enhances the performance of synthetic mRNA switches in cells, Nucleic Acids Research, Volume 48, Issue 6, 6 Apr. 2020, Page e35, and Andries et al., Journal of Controlled Release, Volume 217, 10 Nov. 2015, Pages 337-344, each of which is incorporated herein by reference in its entirety.

In some embodiments, the guide polynucleotide comprises one or more modified nucleotides at the 5′ end and/or the 3′ end of the guide. In some embodiments, the guide polynucleotide comprises two, three, four or more modified nucleosides at the 5′ end and/or the 3′ end of the guide. In some embodiments, the guide polynucleotide comprises two, three, four or more modified nucleosides at the 5′ end and/or the 3′ end of the guide.

In some embodiments, the guide comprises at least about 50%-75% modified nucleotides. In some embodiments, the guide comprises at least about 85% or more modified nucleotides. In some embodiments, at least about 1-5 nucleotides at the 5′ end of the gRNA are modified and at least about 1-5 nucleotides at the 3′ end of the gRNA are modified. In some embodiments, at least about 3-5 contiguous nucleotides at each of the 5′ and 3′ termini of the gRNA are modified. In some embodiments, at least about 20% of the nucleotides present in a direct repeat or anti-direct repeat are modified. In some embodiments, at least about 50% of the nucleotides present in a direct repeat or anti-direct repeat are modified. In some embodiments, at least about 50-75% of the nucleotides present in a direct repeat or anti-direct repeat are modified. In some embodiments, at least about 100 of the nucleotides present in a direct repeat or anti-direct repeat are modified. In some embodiments, at least about 20% or more of the nucleotides present in a hairpin present in the gRNA scaffold are modified. In some embodiments, at least about 50% or more of the nucleotides present in a hairpin present in the gRNA scaffold are modified. In some embodiments, the guide comprises a variable length spacer. In some embodiments, the guide comprises a 20-40 nucleotide spacer. In some embodiments, the guide comprises a spacer comprising at least about 20-25 nucleotides or at least about 30-35 nucleotides. In some embodiments, the spacer comprises modified nucleotides. In some embodiments, the guide comprises two or more of the following:
at least about 1-5 nucleotides at the 5′ end of the gRNA are modified and at least about 1-5 nucleotides at the 3′ end of the gRNA are modified;
at least about 20% of the nucleotides present in a direct repeat or anti-direct repeat are modified;
at least about 50-75% of the nucleotides present in a direct repeat or anti-direct repeat are modified;
at least about 20% or more of the nucleotides present in a hairpin present in the gRNA scaffold are modified;
a variable length spacer; and
a spacer comprising modified nucleotides.

In embodiments, the gRNA contains numerous modified nucleotides and/or chemical modifications (“heavy mods”). Such heavy mods can increase base editing ˜2 fold in vivo or in vitro. In embodiments, the gRNA comprises 2′-O-methyl or phosphorothioate modifications. In an embodiment, the gRNA comprises 2′-O-methyl and phosphorothioate modifications. In an embodiment, the modifications increase base editing by at least about 2 fold.

A guide polynucleotide can comprise one or more modifications to provide a nucleic acid with a new or enhanced feature. A guide polynucleotide can comprise a nucleic acid affinity tag. A guide polynucleotide can comprise synthetic nucleotide, synthetic nucleotide analog, nucleotide derivatives, and/or modified nucleotides.

A gRNA or a guide polynucleotide can also be modified by 5′ adenylate, 5′ guanosine-triphosphate cap, 5′ N₇-Methylguanosine-triphosphate cap, 5′ triphosphate cap, 3′ phosphate, 3′ thiophosphate, 5′ phosphate, 5′ thiophosphate, Cis-Syn thymidine dimer, trimers, C₁₂ spacer, C₃ spacer, C₆ spacer, dSpacer, PC spacer, rSpacer, Spacer 18, Spacer 9, 3′-3′ modifications, 2′-O-methyl thioPACE (MSP), 2′-O-methyl-PACE (MP), and constrained ethyl (S-cEt), 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C₂, psoralen C₆, TINA, 3′ DABCYL, black hole quencher 1, black hole quencher 2, DABCYL SE, dT-DABCYL, IRDye QC-1, QSY-21, QSY-35, QSY-7, QSY-9, carboxyl linker, thiol linkers, 2′-deoxyribonucleoside analog purine, 2′-deoxyribonucleoside analog pyrimidine, ribonucleoside analog, 2′-O-methyl ribonucleoside analog, sugar modified analogs, wobble/universal bases, fluorescent dye label, 2′-fluoro RNA, 2′-O-methyl RNA, methylphosphonate, phosphodiester DNA, phosphodiester RNA, phosphothioate DNA, phosphorothioate RNA, UNA, pseudouridine-5′-triphosphate, 5′-methylcytidine-5′-triphosphate, or any combination thereof.

In some cases, a phosphorothioate enhanced RNA gRNA can inhibit RNase A, RNase T1, calf serum nucleases, or any combinations thereof. These properties can allow the use of PS-RNA gRNAs to be used in applications where exposure to nucleases is of high probability in vivo or in vitro. For example, phosphorothioate (PS) bonds can be introduced between the last 3-5 nucleotides at the 5′- or 3′-end of a gRNA which can inhibit exonuclease degradation. In some cases, phosphorothioate bonds can be added throughout an entire gRNA to reduce attack by endonucleases.

### Fusion Proteins or Complexes Comprising a Nuclear Localization Sequence (NLS)

In some embodiments, the fusion proteins or complexes provided herein further comprise one or more (e.g., 2, 3, 4, 5) nuclear targeting sequences, for example a nuclear localization sequence (NLS). In one embodiment, a bipartite NLS is used. In some embodiments, a NLS comprises an amino acid sequence that facilitates the importation of a protein, that comprises an NLS, into the cell nucleus (e.g., by nuclear transport). In some embodiments, the NLS is fused to the N-terminus or the C-terminus of the fusion protein. In some embodiments, the NLS is fused to the C-terminus or N-terminus of an nCas9 domain or a dCas9 domain. In some embodiments, the NLS is fused to the N-terminus or C-terminus of the Cas 12 domain. In some embodiments, the NLS is fused to the N-terminus or C-terminus of the cytidine or adenosine deaminase. In some embodiments, the NLS is fused to the fusion protein via one or more linkers. In some embodiments, the NLS is fused to the fusion protein without a linker. In some embodiments, the NLS comprises an amino acid sequence of any one of the NLS sequences provided or referenced herein. Additional nuclear localization sequences are known in the art and would be apparent to the skilled artisan. For example, NLS sequences are described in Plank et al., PCT/EP2000/011690, the contents of which are incorporated herein by reference for their disclosure of exemplary nuclear localization sequences.

In some embodiments, the NLS is present in a linker or the NLS is flanked by linkers, for example described herein. A bipartite NLS comprises two basic amino acid clusters, which are separated by a relatively short spacer sequence (hence bipartite-2 parts, while monopartite NLSs are not). The NLS of nucleoplasmin, KR [PAATKKAGQA] KKKK (SEQ ID NO: 191), is the prototype of the ubiquitous bipartite signal: two clusters of basic amino acids, separated by a spacer of about 10 amino acids. The sequence of an exemplary bipartite NLS follows:

(SEQ ID NO: 328)
PKKKRKVEGADKRTADGSEFESPKKKRKV.

In some embodiments, any of the fusion proteins or complexes provided herein comprise an NLS comprising the amino acid sequence EGADKRTADGSEFESPKKKRKV (amino acids 8 to 29 of SEQ ID NO 328). In some embodiments, any of the adenosine base editors provided herein comprise the amino acid sequence EGADKRTADGSEFESPKKKRKV (amino acids 8 to 29 of SEQ ID NO: 328). In some embodiments, the NLS is at a C-terminal portion of the adenosine base editor. In some embodiments, the NLS is at the C-terminus of the adenosine base editor.

### Additional Domains

A base editor described herein can include any domain which helps to facilitate the nucleobase editing, modification or altering of a nucleobase of a polynucleotide. In some embodiments, a base editor comprises a polynucleotide programmable nucleotide binding domain (e.g., Cas9), a nucleobase editing domain (e.g., deaminase domain), and one or more additional domains. In some embodiments, the additional domain can facilitate enzymatic or catalytic functions of the base editor, binding functions of the base editor, or be inhibitors of cellular machinery (e.g., enzymes) that could interfere with the desired base editing result. In some embodiments, a base editor comprises a nuclease, a nickase, a recombinase, a deaminase, a methyltransferase, a methylase, an acetylase, an acetyltransferase, a transcriptional activator, or a transcriptional repressor domain.

In some embodiments, a base editor comprises an uracil glycosylase inhibitor (UGI) domain. In some cases, a base editor is expressed in a cell in trans with a UGI polypeptide. In some embodiments, cellular DNA repair response to the presence of U: G heteroduplex DNA can be responsible for a reduction in nucleobase editing efficiency in cells. In such embodiments, uracil DNA glycosylase (UDG) can catalyze removal of U from DNA in cells, which can initiate base excision repair (BER), mostly resulting in reversion of the U: G pair to a C: G pair. In such embodiments, BER can be inhibited in base editors comprising one or more domains that bind the single strand, block the edited base, inhibit UGI, inhibit BER, protect the edited base, and/or promote repairing of the non-edited strand. Thus, this disclosure contemplates a base editor fusion protein or complex comprising a UGI domain and/or a uracil stabilizing protein (USP) domain.

### Base Editor System

Provided herein are systems, compositions, and methods for editing a nucleobase using a base editor system. In some embodiments, the base editor system comprises (1) a base editor (BE) comprising a polynucleotide programmable nucleotide binding domain and a nucleobase editing domain (e.g., a deaminase domain) for editing the nucleobase; and (2) a guide polynucleotide (e.g., guide RNA) in conjunction with the polynucleotide programmable nucleotide binding domain. In some embodiments, the base editor system is a cytidine base editor (CBE) or an adenosine base editor (ABE). In some embodiments, the polynucleotide programmable nucleotide binding domain is a polynucleotide programmable DNA or RNA binding domain. In some embodiments, the nucleobase editing domain is a deaminase domain. In some embodiments, a deaminase domain can be a cytidine deaminase or an cytosine deaminase. In some embodiments, a deaminase domain can be an adenine deaminase or an adenosine deaminase. In some embodiments, the adenosine base editor can deaminate adenine in DNA. In some embodiments, the base editor is capable of deaminating a cytidine in DNA.

Use of the base editor system provided herein comprises the steps of: (a) contacting a target nucleotide sequence of a polynucleotide (e.g., double- or single stranded DNA or RNA) of a subject with a base editor system comprising a nucleobase editor (e.g., an adenosine base editor or a cytidine base editor) and a guide polynucleotide (e.g., gRNA), wherein the target nucleotide sequence comprises a targeted nucleobase pair; (b) inducing strand separation of said target region; (c) converting a first nucleobase of said target nucleobase pair in a single strand of the target region to a second nucleobase; and (d) cutting no more than one strand of said target region, where a third nucleobase complementary to the first nucleobase base is replaced by a fourth nucleobase complementary to the second nucleobase. It should be appreciated that in some embodiments, step (b) is omitted. In some embodiments, said targeted nucleobase pair is a plurality of nucleobase pairs in one or more genes. In some embodiments, the base editor system provided herein is capable of multiplex editing of a plurality of nucleobase pairs in one or more genes. In some embodiments, the plurality of nucleobase pairs is located in the same gene. In some embodiments, the plurality of nucleobase pairs is located in one or more genes, wherein at least one gene is located in a different locus.

The components of a base editor system (e.g., a deaminase domain, a guide RNA, and/or a polynucleotide programmable nucleotide binding domain) may be associated with each other covalently or non-covalently. For example, in some embodiments, the deaminase domain can be targeted to a target nucleotide sequence by a polynucleotide programmable nucleotide binding domain, optionally where the polynucleotide programmable nucleotide binding domain is complexed with a polynucleotide (e.g., a guide RNA). In some embodiments, a polynucleotide programmable nucleotide binding domain can be fused or linked to a deaminase domain. In some embodiments, a polynucleotide programmable nucleotide binding domain can target a deaminase domain to a target nucleotide sequence by non-covalently interacting with or associating with the deaminase domain. For example, in some embodiments, the nucleobase editing component (e.g., the deaminase component) comprises an additional heterologous portion or domain that is capable of interacting with, associating with, or capable of forming a complex with a corresponding heterologous portion, antigen, or domain that is part of a polynucleotide programmable nucleotide binding domain and/or a guide polynucleotide (e.g., a guide RNA) complexed therewith. In some embodiments, the polynucleotide programmable nucleotide binding domain, and/or a guide polynucleotide (e.g., a guide RNA) complexed therewith, comprises an additional heterologous portion or domain that is capable of interacting with, associating with, or capable of forming a complex with a corresponding heterologous portion, antigen, or domain that is part of a nucleobase editing domain (e.g., the deaminase component). In some embodiments, the additional heterologous portion may be capable of binding to, interacting with, associating with, or forming a complex with a polypeptide. In some embodiments, the additional heterologous portion may be capable of binding to, interacting with, associating with, or forming a complex with a polynucleotide. In some embodiments, the additional heterologous portion may be capable of binding to a guide polynucleotide. In some embodiments, the additional heterologous portion may be capable of binding to a polypeptide linker. In some embodiments, the additional heterologous portion is capable of binding to a polynucleotide linker. An additional heterologous portion may be a protein domain. In some embodiments, an additional heterologous portion comprises a polypeptide, such as a 22 amino acid RNA-binding domain of the lambda bacteriophage antiterminator protein N (N₂₂p), a 2G12 IgG homodimer domain, an ABI, an antibody (e.g. an antibody that binds a component of the base editor system or a heterologous portion thereof) or fragment thereof (e.g. heavy chain domain 2 (CH2) of IgM (MHD2) or IgE (EHD2), an immunoglobulin Fc region, a heavy chain domain 3 (CH3) of IgG or IgA, a heavy chain domain 4 (CH4) of IgM or IgE, an Fab, an Fab2, miniantibodies, and/or ZIP antibodies), a barnase-barstar dimer domain, a Bcl-XL domain, a Calcineurin A (CAN) domain, a Cardiac phospholamban transmembrane pentamer domain, a collagen domain, a Com RNA binding protein domain (e.g. SfMu Com coat protein domain, and SfMu Com binding protein domain), a Cyclophilin-Fas fusion protein (CyP-Fas) domain, a Fab domain, an Fc domain, a fibritin foldon domain, an FK506 binding protein (FKBP) domain, an FKBP binding domain (FRB) domain of mTOR, a foldon domain, a fragment X domain, a GAI domain, a GID1 domain, a Glycophorin A transmembrane domain, a GyrB domain, a Halo tag, an HIV Gp41 trimerisation domain, an HPV45 oncoprotein E7 C-terminal dimer domain, a hydrophobic polypeptide, a K Homology (KH) domain, a Ku protein domain (e.g., a Ku heterodimer), a leucine zipper, a LOV domain, a mitochondrial antiviral-signaling protein CARD filament domain, an MS2 coat protein domain (MCP), a non-natural RNA aptamer ligand that binds a corresponding RNA motif/aptamer, a parathyroid hormone dimerization domain, a PP7 coat protein (PCP) domain, a PSD95-Dlgl-zo-1 (PDZ) domain, a PYL domain, a SNAP tag, a SpyCatcher moiety, a SpyTag moiety, a streptavidin domain, a streptavidin-binding protein domain, a streptavidin binding protein (SBP) domain, a telomerase Sm7 protein domain (e.g. Sm7 homoheptamer or a monomeric Sm-like protein), and/or fragments thereof. In embodiments, an additional heterologous portion comprises a polynucleotide (e.g., an RNA motif), such as an MS2 phage operator stem-loop (e.g., an MS2, an MS2 C-5 mutant, or an MS2 F-5 mutant), a non-natural RNA motif, a PP7 operator stem-loop, an SfMu phate Com stem-loop, a steril alpha motif, a telomerase Ku binding motif, a telomerase Sm7 binding motif, and/or fragments thereof. Non-limiting examples of additional heterologous portions include polypeptides with at least about 85% sequence identity to any one or more of SEQ ID NOs: 380, 382, 384, 386-388, or fragments thereof. Non-limiting examples of additional heterologous portions include polynucleotides with at least about 85% sequence identity to any one or more of SEQ ID NOs: 379, 381, 383, 385, or fragments thereof.

In some instances, components of the base editing system are associated with one another through the interaction of leucine zipper domains (e.g., SEQ ID NOs: 387 and 388). In some cases, components of the base editing system are associated with one another through polypeptide domains (e.g., FokI domains) that associate to form protein complexes containing about, at least about, or no more than about 1, 2 (i.e., dimerize), 3, 4, 5, 6, 7, 8, 9, 10 polypeptide domain units, optionally the polypeptide domains may include alterations that reduce or eliminate an activity thereof.

In some instances, components of the base editing system are associated with one another through the interaction of multimeric antibodies or fragments thereof (e.g., IgG, IgD, IgA, IgM, IgE, a heavy chain domain 2 (CH₂) of IgM (MHD2) or IgE (EHD2), an immunoglobulin Fc region, a heavy chain domain 3 (CH₃) of IgG or IgA, a heavy chain domain 4 (CH₄) of IgM or IgE, an Fab, and an Fab2). In some instances, the antibodies are dimeric, trimeric, or tetrameric. In embodiments, the dimeric antibodies bind a polypeptide or polynucleotide component of the base editing system.

In some cases, components of the base editing system are associated with one another through the interaction of a polynucleotide-binding protein domain(s) with a polynucleotide(s). In some instances, components of the base editing system are associated with one another through the interaction of one or more polynucleotide-binding protein domains with polynucleotides that are self-complementary and/or complementary to one another so that complementary binding of the polynucleotides to one another brings into association their respective bound polynucleotide-binding protein domain(s).

In some instances, components of the base editing system are associated with one another through the interaction of a polypeptide domain(s) with a small molecule(s) (e.g., chemical inducers of dimerization (CIDs), also known as “dimerizers”). Non-limiting examples of CIDs include those disclosed in Amara, et al., “A versatile synthetic dimerizer for the regulation of protein-protein interactions,” PNAS, 94:10618-10623 (1997); and Voß, et al. “Chemically induced dimerization: reversible and spatiotemporal control of protein function in cells,” Current Opinion in Chemical Biology, 28:194-201 (2015), the disclosures of each of which are incorporated herein by reference in their entireties for all purposes. In some embodiments, the base editor inhibits base excision repair (BER) of the edited strand. In some embodiments, the base editor protects or binds the non-edited strand. In some embodiments, the base editor comprises UGI activity or USP activity. In some embodiments, the base editor comprises a catalytically inactive inosine-specific nuclease.

The base editors of the present disclosure can comprise any domain, feature or amino acid sequence which facilitates the editing of a target polynucleotide sequence. For example, in some embodiments, the base editor comprises a nuclear localization sequence (NLS). In some embodiments, an NLS of the base editor is localized between a deaminase domain and a polynucleotide programmable nucleotide binding domain. In some embodiments, an NLS of the base editor is localized C-terminal to a polynucleotide programmable nucleotide binding domain.

Protein domains included in the fusion protein can be a heterologous functional domain. Non-limiting examples of protein domains which can be included in the fusion protein include a deaminase domain (e.g., cytidine deaminase and/or adenosine deaminase), a uracil glycosylase inhibitor (UGI) domain, epitope tags, and reporter gene sequences. In some embodiments, the adenosine base editor (ABE) can deaminate adenine in DNA. In some embodiments, ABE is generated by replacing APOBEC1 component of BE3 with natural or engineered E. coli TadA, human ADAR2, mouse ADA, or human ADAT2. In some embodiments, ABE comprises an evolved TadA variant. In some embodiments, the base editor is ABE8.1, which comprises or consists essentially of the following sequence or a fragment thereof having adenosine deaminase activity: SEQ ID NO: 331. Other ABE8 sequences are provided in the attached sequence listing (SEQ ID NOs: 332-354).

In some embodiments, the base editor includes an adenosine deaminase variant comprising an amino acid sequence, which contains alterations relative to an ABE 7*10 reference sequence, as described herein. The term “monomer” as used in Table 7 refers to a monomeric form of TadA*7.10 comprising the alterations described. The term “heterodimer” as used in Table 7 refers to the specified wild-type E. coli TadA adenosine deaminase fused to a TadA*7.10 comprising the alterations as described.

TABLE 7
Adenosine Deaminase Base Editor Variants

| | Adenosine | |
|---|---|---|
| ABE | Deaminase | Adenosine Deaminase Description |
| ABE-605m | MSP605 | monomer_TadA*7.10 + V82G + Y147T + Q154S |
| ABE-680m | MSP680 | monomer_TadA*7.10 + I76Y + V82G + Y147T + Q154S |
| ABE-823m | MSP823 | monomer_TadA*7.10 + L36H + V82G + Y147T + Q154S + |
| | | N157K |
| ABE-824m | MSP824 | monomer_TadA*7.10 + V82G + Y147D + F149Y + Q154S + |
| | | D167N |
| ABE-825m | MSP825 | monomer_TadA*7.10 + L36H + V82G + Y147D + F149Y + |
| | | Q154S + N157K + D167N |
| ABE-827m | MSP827 | monomer_TadA*7.10 + L36H + I76Y + V82G + Y147T + Q154S + |
| | | N157K |
| ABE-828m | MSP828 | monomer_TadA*7.10 + I76Y + V82G + Y147D + F149Y + Q154S + |
| | | D167N |
| ABE-829m | MSP829 | monomer_TadA*7.10 + L36H + I76Y + V82G + Y147D + F149Y + |
| | | Q154S + N157K + D167N |
| ABE-605d | MSP605 | heterodimer_(WT) + (TadA*7.10 + V82G + Y147T + Q154S) |
| ABE-680d | MSP680 | heterodimer_(WT) + (TadA*7.10 + I76Y + V82G + Y147T + |
| | | Q154S) |
| ABE-823d | MSP823 | heterodimer_(WT) + (TadA*7.10 + L36H + V82G + Y147T + |
| | | Q154S + N157K) |
| ABE-824d | MSP824 | heterodimer_(WT) + (TadA*7.10 + V82G + Y147D + F149Y + |
| | | Q154S + D167N) |
| ABE-825d | MSP825 | heterodimer_(WT) + (TadA*7.10 + L36H + V82G + Y147D + |
| | | F149Y + Q154S + N157K + D167N) |
| ABE-827d | MSP827 | heterodimer_(WT) + (TadA*7.10 + L36H + I76Y + V82G + Y147T + |
| | | Q154S + N157K) |
| ABE-828d | MSP828 | heterodimer_(WT) + (TadA*7.10 + I76Y + V82G + Y147D + |
| | | F149Y + Q154S + D167N) |
| ABE-829d | MSP829 | heterodimer_(WT) + (TadA*7.10 + L36H + I76Y + V82G + Y147D + |
| | | F149Y + Q154S + N157K + D167N) |

In some embodiments, the base editor comprises a domain comprising all or a portion (e.g., a functional portion) of a uracil glycosylase inhibitor (UGI) or a uracil stabilizing protein (USP) domain.

### Linkers

In certain embodiments, linkers may be used to link any of the peptides or peptide domains of the disclosure. The linker may be as simple as a covalent bond, or it may be a polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.).

In some embodiments, any of the fusion proteins provided herein, comprise a cytidine or adenosine deaminase and a Cas9 domain that are fused to each other via a linker. Various linker lengths and flexibilities between the cytidine or adenosine deaminase and the Cas9 domain can be employed (e.g., ranging from very flexible linkers of the form (GGGS) n (SEQ ID NO: 246), (GGGGS) n (SEQ ID NO: 247), and (G) n to more rigid linkers of the form (EAAAK) n (SEQ ID NO: 248), (SGGS) n (SEQ ID NO: 355), SGSETPGTSESATPES(SEQ ID NO: 249) (see, e.g., Guilinger JP, et al. Fusion of catalytically inactive Cas9 to FokI nuclease improves the specificity of genome modification. Nat. Biotechnol. 2014; 32 (6): 577-82; the entire contents are incorporated herein by reference) and (XP) n) in order to achieve the optimal length for activity for the cytidine or adenosine deaminase nucleobase editor. In some embodiments, n is 1, 2, 3, 4, 5, 6, 7, 8, 9, 10, 11, 12, 13, 14, or 15. In some embodiments, the linker comprises a (GGS) n motif, wherein n is 1, 3, or 7. In some embodiments, cytidine deaminase or adenosine deaminase and the Cas9 domain of any of the fusion proteins provided herein are fused via a linker comprising the amino acid sequence SGSETPGTSESATPES(SEQ ID NO: 249), which can also be referred to as the XTEN linker.

In some embodiments, the domains of the base editor are fused via a linker that comprises the amino acid sequence of:

(SEQ ID NO: 356)
SGGSSGSETPGTSESATPESSGGS,
(SEQ ID NO: 357)
SGGSSGGSSGSETPGTSESATPESSGGSSGGS,
or
(SEQ ID NO: 358)
GGSGGSPGSPAGSPTSTEEGTSESATPESGPGTSTEPSEGSAPGSPAGSP
TSTEEGTSTEPSEGSAPGTSTEPSEGSAPGTSESATPESGPGSEPATSGG
SGGS.

In some embodiments, domains of the base editor are fused via a linker comprising the amino acid sequence SGSETPGTSESATPES(SEQ ID NO: 249), which may also be referred to as the XTEN linker. In some embodiments, a linker comprises the amino acid sequence SGGS(SEQ ID NO: 355). In some embodiments, the linker is 24 amino acids in length. In some embodiments, the linker comprises the amino acid sequence SGGSSGGSSGSETPGTSESATPES(SEQ ID NO: 359). In some embodiments, the linker is 40 amino acids in length. In some embodiments, the linker comprises the amino acid sequence: SGGSSGGSSGSETPGTSESATPESSGGSSGGSSGGSSGGS(SEQ ID NO: 360). In some embodiments, the linker is 64 amino acids in length. In some embodiments, the linker comprises the amino acid sequence: SGGSSGGSSGSETPGTSESATPESSGGSSGGSSGGSSGGSSGSETPGTSESATPESSGGSSG GS(SEQ ID NO: 361). In some embodiments, the linker is 92 amino acids in length. In some embodiments, the linker comprises the amino acid sequence:

(SEQ ID NO: 362)
PGSPAGSPTSTEEGTSESATPESGPGTSTEPSEGSAPGSPAGSPTSTEEG
TSTEPSEGSAPGTSTEPSEGSAPGTSESATPESGPGSEPATS.

In some embodiments, a linker comprises a plurality of proline residues and is 5-21, 5-14, 5-9, 5-7 amino acids in length, e.g., PAPAP(SEQ ID NO: 363), PAPAPA (SEQ ID NO: 364), PAPAPAP(SEQ ID NO: 365), PAPAPAPA (SEQ ID NO: 366), P(AP)₄ (SEQ ID NO: 367), P(AP)₇ (SEQ ID NO: 368), P(AP)₁₀ (SEQ ID NO: 369) (see, e.g., Tan J, Zhang F, Karcher D, Bock R. Engineering of high-precision base editors for site-specific single nucleotide replacement. Nat Commun. 2019 Jan. 25;10 (1): 439; the entire contents are incorporated herein by reference). Such proline-rich linkers are also termed “rigid” linkers.

Nucleic Acid Programmable DNA Binding Proteins with Guide RNAs

Provided herein are compositions and methods for base editing in cells. Further provided herein are compositions comprising a guide polynucleotide sequence, e.g., a guide RNA sequence, or a combination of 2, 3, 4, 5, 6, 7, 8, 9, 10, 11, 12, 13, 14, 15, 16, 17, 18, 19, 20, or more guide RNAs as provided herein. In some embodiments, a composition for base editing as provided herein further comprises a polynucleotide that encodes a base editor, e.g., a C-base editor or an A-base editor. For example, a composition for base editing may comprise a mRNA sequence encoding a BE, a BE4, an ABE, and a combination of one or more guide RNAs as provided. A composition for base editing may comprise a base editor polypeptide and a combination of one or more of any guide RNAs provided herein. Such a composition may be used to effect base editing in a cell through different delivery approaches, for example, electroporation, nucleofection, viral transduction or transfection. In some embodiments, the composition for base editing comprises an mRNA sequence that encodes a base editor and a combination of one or more guide RNA sequences provided herein for electroporation.

Some aspects of this disclosure provide systems comprising any of the fusion proteins or complexes provided herein, and a guide RNA bound to a nucleic acid programmable DNA binding protein (napDNAbp) domain (e.g., a Cas9 (e.g., a dCas9, a nuclease active Cas9, or a Cas9 nickase) or Cas12) of the fusion protein or complex. These complexes are also termed ribonucleoproteins (RNPs). In some embodiments, the guide nucleic acid (e.g., guide RNA) is from 15-100 nucleotides long and comprises a sequence of at least 10 contiguous nucleotides that is complementary to a target sequence. In some embodiments, the target sequence is a DNA sequence. In some embodiments, the target sequence is an RNA sequence. In some embodiments, the target sequence is a sequence in the genome of a bacteria, yeast, fungi, insect, plant, or animal. In some embodiments, the target sequence is a sequence in the genome of a human. In some embodiments, the 3′ end of the target sequence is immediately adjacent to a canonical PAM sequence (NGG). In some embodiments, the 3′ end of the target sequence is immediately adjacent to a non-canonical PAM sequence (e.g., a sequence listed in Table 3 or 5′—NAA-3′). In some embodiments, the guide nucleic acid (e.g., guide RNA) is complementary to a sequence in a gene of interest (e.g., a gene associated with a disease or disorder).

Some aspects of this disclosure provide methods of using the fusion proteins, or complexes provided herein. For example, some aspects of this disclosure provide methods comprising contacting a DNA molecule with any of the fusion proteins or complexes provided herein, and with at least one guide RNA, wherein the guide RNA is about 15-100 nucleotides long and comprises a sequence of at least 10 contiguous nucleotides that is complementary to a target sequence.

The domains of the base editor disclosed herein can be arranged in any order.

A defined target region can be a deamination window. A deamination window can be the defined region in which a base editor acts upon and deaminates a target nucleotide. In some embodiments, the deamination window is within a 2, 3, 4, 5, 6, 7, 8, 9, or 10 base regions. In some embodiments, the deamination window is 5, 6, 7, 8, 9, 10, 11, 12, 13, 14, 15, 16, 17, 18, 19, 20, 21, 22, 23, 24, or 25 bases upstream of the PAM.

The base editors of the present disclosure can comprise any domain, feature or amino acid sequence which facilitates the editing of a target polynucleotide sequence.

### Methods of Using Fusion Proteins or Complexes Comprising a Cytidine or Adenosine Deaminase and a Cas9 Domain

Some aspects of this disclosure provide methods of using the fusion proteins, or complexes provided herein. For example, some aspects of this disclosure provide methods comprising contacting a DNA molecule with any of the fusion proteins or complexes provided herein, and with at least one guide RNA described herein.

In some embodiments, a fusion protein or complex of the disclosure is used for editing a target gene of interest. In particular, a cytidine deaminase or adenosine deaminase nucleobase editor described herein is capable of making multiple mutations within a target sequence. These mutations may affect the function of the target. For example, when a cytidine deaminase or adenosine deaminase nucleobase editor is used to target a regulatory region the function of the regulatory region is altered and the expression of the downstream protein is reduced or eliminated.

### Base Editor Efficiency

In some embodiments, the purpose of the methods provided herein is to alter a gene and/or gene product via gene editing. The nucleobase editing proteins provided herein can be used for gene editing-based human therapeutics in vitro or in vivo. It will be understood by the skilled artisan that the nucleobase editing proteins provided herein, e.g., the fusion proteins or complexes comprising a polynucleotide programmable nucleotide binding domain (e.g., Cas9) and a nucleobase editing domain (e.g., an adenosine deaminase domain or a cytidine deaminase domain) can be used to edit a nucleotide from A to Gor C to T.

Advantageously, base editing systems as provided herein provide genome editing without generating double-strand DNA breaks, without requiring a donor DNA template, and without inducing an excess of stochastic insertions and deletions as CRISPR may do. In some embodiments, the present disclosure provides base editors that efficiently generate an intended mutation, such as a STOP codon, in a nucleic acid (e.g., a nucleic acid within a genome of a subject) without generating a significant number of unintended mutations, such as unintended point mutations.

The base editors of the disclosure advantageously modify a specific nucleotide base encoding a protein without generating a significant proportion of indels (i.e., insertions or deletions). Such indels can lead to frame shift mutations within a coding region of a gene.

In some embodiments, the base editors provided herein are capable of generating a ratio of intended mutations to indels (i.e., intended point mutations: unintended point mutations) that is greater than 1:1. In some embodiments, the base editors provided herein are capable of generating a ratio of intended mutations to indels that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 200:1, at least 300:1, at least 400:1, at least 500:1, at least 600:1, at least 700:1, at least 800:1, at least 900:1, or at least 1000:1, or more. The number of intended mutations and indels may be determined using any suitable method.

In some embodiments, the base editors provided herein can limit formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor. In some embodiments, any of the base editors provided herein can limit the formation of indels at a region of a nucleic acid to less than 1%, less than 1.5%, less than 2%, less than 2.5%, less than 3%, less than 3.5%, less than 4%, less than 4.5%, less than 5%, less than 6%, less than 7%, less than 8%, less than 9%, less than 10%, less than 12%, less than 15%, or less than 20%.

Base editing is often referred to as a “modification”, such as, a genetic modification, a gene modification and modification of the nucleic acid sequence and is clearly understandable based on the context that the modification is a base editing modification. A base editing modification is therefore a modification at the nucleotide base level, for example as a result of the deaminase activity discussed throughout the disclosure, which then results in a change in the gene sequence and may affect the gene product.

In some embodiments, the modification, e.g., single base edit results in about or at least about a 10%, 15%, 20%, 25%, 30%, 35%, 40%, 45%, 50%, 55%, 60%, 65%, 70%, 75%, 80%, 85%, 90%, 95%, 99%, or 100% reduction, or reduction to an undetectable level, of the gene targeted expression.

The disclosure provides adenosine deaminase variants (e.g., ABE8 variants) that have increased efficiency and specificity. In particular, the adenosine deaminase variants described herein are more likely to edit a desired base within a polynucleotide and are less likely to edit bases that are not intended to be altered (e.g., “bystanders”).

In some embodiments, any of the base editing system comprising one of the ABE8 base editor variants described herein has reduced bystander editing or mutations by at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or 99% compared to a base editor system comprising an ABE7 base editor, e.g., ABE7.10.

In some embodiments, any of the ABE8 base editor variants described herein has higher base editing efficiency compared to the ABE7 base editors. In some embodiments, any of the ABE8 base editor variants described herein have at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 99%, 100%, 105%, 110%, 115%, 120%, 125%, 130%, 135%, 140%, 145%, 150%, 155%, 160%, 165%, 170%, 175%, 180%, 185%, 190%, 195%, 200%, 210%, 220%, 230%, 240%, 250%, 260%, 270%, 280%, 290%, 300%, 310%, 320%, 330%, 340%, 350%, 360%, 370%, 380%, 390%, 400%, 450%, or 500% higher base editing efficiency compared to an ABE7 base editor, e.g., ABE7.10.

The ABE8 base editor variants described herein may be delivered to a host cell via a plasmid, a vector, a LNP complex, or an mRNA. In some embodiments, any of the ABE8 base editor variants described herein is delivered to a host cell as an mRNA.

In some embodiments, the method described herein, for example, the base editing methods has minimum to no off-target effects. In some embodiments, the method described herein, for example, the base editing methods, has minimal to no chromosomal translocations.

In some embodiments, the base editing method described herein results in about 91%, 92%, 93%, 94%, 95%, 96%, 97%, 98%, 99% or 100% of a cell population that have been successfully edited.

In some embodiments, the percent of viable cells in a cell population following a base editing intervention is greater than at least 60%, 70%, 80%, or 90% of the starting cell population at the time of the base editing event. In some embodiments, the percent of viable cells in a cell population following editing is about 70%. In some embodiments, the percent of viable cells in a cell population following editing is about 75%. In some embodiments, the percent of viable cells in a cell population following editing is about 80%. In some embodiments, the percent of viable cells in a cell population as described above is about 85%. In some embodiments, the percent of viable cells in a cell population as described above is about 90%, or about 91%, 92%, 93%, 94% 95%, 96%, 97%, 98%, 99%, or 100% of the cells in the population at the time of the base editing event.

In embodiments, the cell population is a population of cells contacted with a base editor, complex, or base editor system of the present disclosure.

The number of intended mutations and indels can be determined using any suitable method, for example, as described in International PCT Application Nos. PCT/US2017/045381 (WO2018/027078) and PCT/US2016/058344 (WO2017/070632); Komor, A. C., et al., “Programmable editing of a target base in genomic DNA without double-stranded DNA cleavage” Nature 533, 420-424 (2016); Gaudelli, N.M., et al., “Programmable base editing of A.T to G.C in genomic DNA without DNA cleavage” Nature 551, 464-471 (2017); and Komor, A. C., et al., “Improved base excision repair inhibition and bacteriophage Mu Gam protein yields C: G-to-T: A base editors with higher efficiency and product purity” Science Advances 3: eaao4774 (2017); the entire contents of which are hereby incorporated by reference.

In some embodiments, to calculate indel frequencies, sequencing reads are scanned for exact matches to two 10-bp sequences that flank both sides of a window in which indels can occur. If no exact matches are located, the read is excluded from analysis. If the length of this indel window exactly matches the reference sequence the read is classified as not containing an indel. If the indel window is two or more bases longer or shorter than the reference sequence, then the sequencing read is classified as an insertion or deletion, respectively. In some embodiments, the base editors provided herein can limit formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor.

### Multiplex Editing

In some embodiments, the base editor system provided herein is capable of multiplex editing of a plurality of nucleobase pairs in one or more genes or polynucleotide sequences. In some embodiments, the plurality of nucleobase pairs is located in the same gene or in one or more genes, wherein at least one gene is located in a different locus. In some embodiments, the multiplex editing comprises one or more guide polynucleotides. In some embodiments, the multiplex editing comprises one or more base editor systems. In some embodiments, the multiplex editing comprises one or more base editor systems with a single guide polynucleotide or a plurality of guide polynucleotides. In some embodiments, the multiplex editing comprises one or more guide polynucleotides with a single base editor system. It should be appreciated that the characteristics of the multiplex editing using any of the base editors as described herein can be applied to any combination of methods using any base editor provided herein. It should also be appreciated that the multiplex editing using any of the base editors as described herein can comprise a sequential editing of a plurality of nucleobase pairs.

In some embodiments, the base editor system capable of multiplex editing of a plurality of nucleobase pairs in one or more genes comprises one of ABE7, ABE8, and/or ABE9 base editors.

### Delivery Systems

### Nucleic Acid-Based Delivery of Base Editor Systems

Nucleic acid molecules encoding a base editor system according to the present disclosure can be administered to subjects or delivered into cells in vitro or in vivo by art-known methods or as described herein. For example, a base editor system comprising a deaminase (e.g., cytidine or adenine deaminase) can be delivered by vectors (e.g., viral or non-viral vectors), or by naked DNA, DNA complexes, lipid nanoparticles, or a combination of the aforementioned compositions. A base editor system may be delivered to a cell using any methods available in the art including, but not limited to, physical methods (e.g., electroporation, particle gun, calcium phosphate transfection), viral methods, non-viral methods (e.g., liposomes, cationic methods, lipid nanoparticles, polymeric nanoparticles), or biological non-viral methods (e.g., attenuated bacterial, engineered bacteriophages, mammalian virus-like particles, biological liposomes, erythrocyte ghosts, exosomes).

Nanoparticles, which can be organic or inorganic, are useful for delivering a base editor system or component thereof. Nanoparticles are well known in the art and any suitable nanoparticle can be used to deliver a base editor system or component thereof, or a nucleic acid molecule encoding such components. In one example, organic (e.g., lipid and/or polymer) nanoparticles are suitable for use as delivery vehicles in certain embodiments of this disclosure. Non-limiting examples of lipid nanoparticles suitable for use in the methods of the present disclosure include those described in International Patent Application Publications No. WO2022140239, WO2022140252, WO2022140238, WO2022159421, WO2022159472, WO2022159475, WO2022159463, WO2021113365, and WO2021141969, the disclosures of each of which is incorporated herein by reference in its entirety for all purposes.

### Lipid Nanoparticle (LNP)Compositions

The pharmaceutical compositions for gene modification described herein may be encapsulated in lipid nanoparticles (LNP). As used herein, a “lipid nanoparticle (LNP) composition” or a “nanoparticle composition” is a composition comprising one or more described lipids. LNP compositions or formulations, as contemplated herein, are typically sized on the order of micrometers or smaller and may include a lipid bilayer. Nanoparticle compositions encompass lipid nanoparticles (LNPs), liposomes (e.g., lipid vesicles), and lipoplexes. For example, a nanoparticle composition or formulation as contemplated herein may be a liposome having a lipid bilayer with a diameter of 500 nm or less. A LNP as described herein may have a mean diameter of from about 1 nm to about 2500 nm, from about 10 nm to about 1500 nm, from about 20 nm to about 1000 nm, from about 30 nm to about 150 nm, from about 40 nm to about 150 nm, from about 50 nm to about 150 nm, from about 60 nm to about 130 nm, from about 50 nm to 90 nm, from about 55 nm to 85 nm, from about 55 nm to 75 nm, from about 50 nm to about 80 nm, from about 60 nm to about 80 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, or from about 70 nm to about 80 nm. The LNPs described herein can have a mean diameter of about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, or greater. In one embodiment the mean diameter of the LNP is about 70 nm +/−20 nm, 70 nm +/−10 nm, 70 nm +/−5 nm. The LNPs described herein can be substantially non-toxic.

Lipid nanoparticles and components thereof suitable for use in embodiments of the present disclosure include those disclosed in any of International Patent Application Publications No. WO 2022/140239, WO 2022/140252, WO 2022/140238, WO 2022/159421, WO 2022/159472, WO 2022/159475, WO 2022/159463, WO 2021/113365, and WO 2021/141969, the disclosures of each of which are incorporated herein by reference in their entireties for all purposes.

Lipid nanoparticles (LNPs) employ a non-viral drug delivery mechanism that is capable of passing through blood vessels and reaching hepatocytes [Am. J. Pathol. 2010, 176,14-21]. Apolipoprotein E (ApoE) proteins are capable of binding to the LNPs post PEG-lipid diffusion from the LNP surface with a near neutral charge in the blood stream, and thereby function as an endogenous ligand against hepatocytes, which express the low-density lipoprotein receptor (LDLr) [Mol. Ther., 2010, 18, 1357-1364.]. Control the efficient hepatic delivery of LNP include: 1) effective PEG-lipid shedding from LNP surface in blood serum and 2) ApoE binding to the LNP. Endogenous ApoE-mediated LDLr-dependent LNP delivery route is unavailable or less effective path to achieve LNP-based hepatic gene delivery in patient populations that LDLr deficient.

Efficient delivery to cells requires specific targeting and substantial protection from the extracellular environment, particularly serum proteins. One method of achieving specific targeting is to conjugate a targeting moiety to an active agents or pharmaceutical effector such as a nucleic acid agent, thereby directing the active agent or pharmaceutical effector to particular cells or tissues depending on the specificity of the targeting moiety. One way a targeting moiety can improve delivery is by receptor mediated endocytotic activity. This mechanism of uptake involves the movement of nucleic acid agent bound to membrane receptors into the interior of an area that is enveloped by the membrane via invagination of the membrane structure or by fusion of the delivery system with the cell membrane. This process is initiated via activation of a cell surface or membrane receptor following binding of a specific ligand to the receptor. Receptor-mediated endocytotic systems include those that recognize sugars such as galactose, mannose, mannose-6-phosphate, peptides and proteins such as transferrin, asialoglycoprotein, vitamin B12, insulin and epidermal growth factor (EGF). Lipophilic moieties, such as cholesterol or fatty acids, when attached to highly hydrophilic molecules such as nucleic acids can substantially enhance plasma protein binding and consequently circulation half-life. Lipophilic conjugates can also be used in combination with the targeting ligands in order to improve the intracellular trafficking of the targeted delivery approach.

The Asialoglycoprotein receptor (ASGP-R) is a high-capacity receptor, which is abundant on hepatocytes. The ASGP-R shows a 50-fold higher affinity for N-Acetyl-D-Galactosylamine (GalNAc) than D-Gal. LNPs comprising receptor targeting conjugates, may be used to facilitate targeted delivery of the drug substances described herein. The LNPs may include one or more receptor targeting moiety on the surface or periphery of the particle at specified or engineered surface density ranging from relatively low to relatively high surface density. The receptor targeting conjugate may comprise a targeting moiety (or ligand), a linker, and a lipophilic moiety that is connected to the targeting moiety. In some embodiments, the receptor targeting moiety (or ligand) targets a lectin receptor. In some embodiments, the lectin receptor is asialoglycoprotein receptor (ASGPR). In some embodiments the receptor targeting moiety is GalNAc or a derivative GalNAc that targets ASGPR. In one aspect the receptor targeting conjugate comprises of one GalNAc moiety or derivative thereof. In another aspect, the receptor targeting conjugate comprises of two different GalNAc moieties or derivative thereof. In another aspect, the receptor targeting conjugate comprises of three different GalNAc moieties or derivative thereof. In another aspect, the receptor targeting conjugate is lipophilic. In some embodiments, the receptor targeting conjugate comprises one or more GalNAc moieties and one or more lipid moieties, i.e., GalNAc-Lipid. In some embodiments, the receptor targeting conjugate is a GalNAc-Lipid.

Described herein are (i) LNP compositions comprising an amino lipid, a phospholipid, a PEG lipid, a cholesterol, or a derivative thereof, a payload, or any combination thereof and (ii) LNP compositions comprising an amino lipid, a phospholipid, a PEG-lipid, a cholesterol, a GalNAc-Lipid or a derivative thereof, a payload, or any combination thereof. Each component is described in more detail below.

In the preparation of LNP compositions comprising the excipients amino lipid, phospholipid, PEG-Lipid and cholesterol, a desired molar ratio of the four excipients is dissolved in a water miscible organic solvent, ethanol for example. The homogenous lipid solution is then rapidly in-line mixed with an aqueous buffer with acidic pH ranging from 4 to 6.5 containing nucleic acid payload to form the lipid nanoparticle (LNP) encapsulating the nucleic acid payload(s). After rapid in-line mixing the LNPs thus formed undergo further downstream processing including concentration and buffer exchange to achieve the final LNP pharmaceutical composition with near neutral pH for administration into cell line or animal diseases model for evaluation, or to administer to human subjects.

For the preparation of GalNAc-LNP pharmaceutical composition the GalNAc-Lipid is mixed with the four lipid excipients in the water miscible organic solvent prior to the preparation of the GalNAc-LNP. The preparation of the GalNAc-LNP pharmaceutical composition then follow the same steps as described for the LNP pharmaceutical composition. The mol % of the GalNAc-Lipid in the GalNAc-LNP preparation ranges from 0.001 to 2.0 of the total excipients.

For both LNP and GalNAc-LNP preparation the payload comprises of a guide RNA targeting the TTR gene and an mRNA encoding a base editor protein. In some embodiments, the guide RNA to mRNA ratio in the acidic aqueous buffer and in the final formulation is 6:1, 5:1, 4:1, 3:1, 2.5:1, 2:1, 1.5:1, 1:1, 1:1.5, 1:2, 1:2.5, 1:3, 1:5 or 1:6 by wight. In some embodiment the said mRNA encodes adenosine base editor protein. In some other embodiments the said mRNA encodes cytosine or cytidine base editor protein.

In some embodiments, an LNP composition may be prepared as described in U.S. patent application Ser. No. 17/192,709, entitled COMPOSITIONS AND METHODS FOR TARGETED RNA DELIVERY, filed on 4 Mar. 2021, claiming the benefit of U.S. Provisional Patent Application Nos. 62/984,866 (filed on 4 Mar. 2020) and 63/078,982 (filed on 16 Sep. 2020), naming Kallanthottathil G. Rajeev as an inventor and Verve Therapeutics, Inc. as the applicant, which application is hereby incorporated herein by reference in its entirety.

### Amino Lipids

In some embodiments, the LNP composition comprises an amino lipid. In some embodiments, the cationic lipid is an ionizable lipid. In some embodiments, the amino lipid (e.g., an ionizable lipid) is a cationic lipid. In some embodiments, the amino lipid (e.g., an ionizable and/or cationic lipid) comprises one or more nitrogen atoms. Exemplary, non-limiting amino lipids suitable for the compositions described herein include those described herein.

### Formula (I)

In one aspect, disclosed herein is an amino lipid having the structure of Formula (I), or a pharmaceutically acceptable salt or solvate thereof,

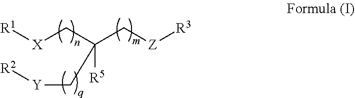

wherein
each of R¹ and R² is independently C₃-C₂₂ alkyl, C₃-C₂₂ alkenyl, C₃-C₈ cycloalkyl, —

C₂-C₁₀ allkylene-L-R⁶, or

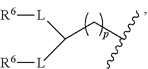

wherein each of the alkyl, alkylene, alkenyl, and cycloalkyl is independently substituted or unsubstituted;
each of X, Y, and Z is independently —C(═O)NR⁴—, —NR⁴C(═O)—, —C(═O)O—, —OC(═O)—, —OC(═O)O—, —NR⁴C(═O)O—, —OC(═O)NR⁴—, —NR⁴C(═O)NR⁴—, —NR⁴C(═NR⁴)NR⁴—, —C(═S)NR⁴—, —NR⁴° C. (═S)—, —C(═O)O—, —OC(═S)—, OC(═S)O—, —NR⁴C(═S)O—, —OC(═S)NR⁴—, —NR⁴C(═S)NR⁴—, —C(═O)S—, —SC(═O)—, —OC(═O)S—, —NR⁴C(═O)S—, —SC(═O)NR⁴—, —C(═S)S—, —SC(═S)—, —SC(═S)O—, —NR⁴C(═S)S—, —SC(═S)NR⁴—, —C(═S)S—, —SC(═S)—, —SC(═O)S—, —SC(═S)S—, —NR⁴C(═S)S—, —SC(═S)NR⁴—O, S, or a bond;
each of L is independently-C(═O)NR⁴—, —NR⁴° C. (═O)—, —C(═O)O—, —OC(═O)O—, —NR⁴C(═O)O—, —OC(═O)NR⁴—, —NR⁴C(═O)NR⁴—, —NR⁴C(═NR⁴)NR⁴—, —C(═S)NR⁴—, —NR⁴C(═S)—, —C(═O)O—, —OC(═S)—, OC(═S)O—, —NR⁴C(═S)O—, —OC(═S)NR⁴—, —NR⁴C(═S)NR⁴—, —C(═O)S—, SC(═O)—, —OC(═O)S—, —NR⁴C(═O)S—, —SC(═O)NR⁴—, —C(═S)S—, —SC(═S)—, —SC(═S)O—, —NR⁴C(═S)S—, —SC(═S)NR⁴—, —C(═S)S—, —SC(═S)—, —SC(═O)S—, —SC(═S)S—, —NR⁴C(═S)S—, —SC(═S)NR⁴—, O, S, —C₁-C₁₀ alkylene-O—, —C₁-C₁₀ alkylene-C(═O)O—, —C₁-C₁₀ alkylene-OC(═O)—, or a bond, wherein the alkylene is substituted or unsubstituted;
R³ is —C₀-C₁₀ alkylene —NR⁷R⁸, —C₀-C₁₀ alkylene-heterocycloalkyl, or —C₀-C₁₀ alkylene-heterocycloaryl, wherein the alkylene, heterocycloalkyl and heterocycloaiyl is independently substituted or unsubstituted; each of R⁴ is independently hydrogen or substituted or unsubstituted C₁-C₆ alkyl;
R⁵ is hydrogen or substituted or unsubstituted C₁-C₆ alkyl;
each of R⁶ is independently substituted or unsubstituted C₃-C₂₂ alkyl or substituted or unsubstituted C₃-C₂₂ alkenyl;
each of R⁷ and R⁸ is independently hydrogen or substituted or unsubstituted C₁-C₆ alkyl, or R⁷ and R⁸ taken together with the nitrogen to which they are attached form a substituted or unsubstituted C₂-C₆ heterocyclyl;
p is an integer selected from 1 to 10; and
each of n, m, and q is independently 0, 1, 2, 3, 4, or 5.

In some embodiments of Formula (I), if the structure carries more than one asymmetric C-atom, each asymmetric C-atom independently represents racemic, chirally pure R and/or chirally pure S isomer, or a combination thereof.

In some embodiments, each of n, in, and q in Formula (I) is independently 0, 1, 2, or 3. In some embodiments, each of n, m, and q in Formula (I) is 1.

### Formula (Ia)

In some embodiments, the compound of Formula (I) has a structure of Formula (Ia), or a pharmaceutically acceptable salt or pharmaceutically acceptable solvate thereof:

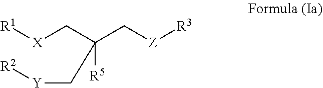

wherein
each of R¹ and R² is independently C₃-C₂₂ alkyl, C₃-C₂₂ alkenyl, C₃-C₈ cycloalkyl, —C₂-C₁₀ alkylene-L-R⁶, or

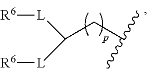

wherein each of the alkyl, alkylene, alkenyl, and cycloalkyl is independently substituted or unsubstituted;
each of X, Y, and Z is independently C(═O)NR⁴—, —NR⁴C(D)—, —C(═O)O—, —OC(═O)—, —OC(═O)O—, —NR⁴C(═O)O—, —OC(═O)NR⁴—, —NR⁴C═O)NR⁴—, —NR⁴C(═NR⁴)NR⁴—, —C(═S)NR⁴—, —NR⁴C(═S)—, —C(E)O—, —OC(═S)—, OC(═S)O—, —NR⁴C(═S)O—, —OC(═S)NR⁴—, —NR⁴C(═S)NR⁴—, —C(═O)S—, —SC(═O)—, —OC(═O)S—, —NR⁴C(═O)S—, —SC(═O)NR⁴—, —C(═S)S—, —SC(═S)—, —SC(═S)O—, —NR⁴C(═S)S—, —SC(═S)NR⁴—, —C(═S)S—, —SC(═S)—, —SC(═O)S—, —SC(═S)S—, —NR⁴C(═S)S—, —SC(═S)NR⁴—, O, S, —C₁-C₁₀ alkylene-O—, or a bond, wherein the alkylene is substituted or unsubstituted;
each of L is independently —C(═O)NR⁴—, —NR⁴C(═O)—, —C(═O)O—, —OC(═O)—, —OC(═O)O—, —NR⁴C(═O)O—, —OC(═O)NR⁴—, —NR⁴C(═O)NR⁴—, —NR⁴° C. (═NR⁴)NR⁴—, —C(═S)NR⁴—, —NR⁴° C. (═S)—, —C(═O)O—, —OC(═S)—, OC(═S)O—, —NR⁴C(═S)O—, —OC(═S)NR⁴—, —NR⁴C(═S)NR⁴—, —C(═O)S—, —SC(═O)—, —OC(═O)S—, —NR⁴C(═O)S—, —SC(═O)NR⁴—, —C(═S)S—, —SC(═S)—, —SC(═S)O—, —NR⁴C(═S)S—, —SC(═S)NR⁴—, —C(═S)S—, —SC(═S)—, —SC(═O)S—, —SC(═S)S—, —NR⁴C(═S)S—, —SC(═S)NR⁴—, O, S, —C₁-C₁₀ alkylene-O—, —C₁-C₁₀ alkylene-C(═O)O—, —C₁-C₁₀ alkylene-OC(═O)—, or a bond, wherein the alkylene is substituted or unsubstituted;
R³ is —C₀-C₁₀ alkylene-NR⁷R⁸, —C₀-C₁₀ alkylene-heterocycloalkyl, or —C₀-C₁₀ alkylene-heterocyclowyl, wherein the alkylene, heterocycloalkyl and heterocycloaryl is independently substituted or unsubstituted;
each of R⁴ is independently hydrogen or substituted or unsubstituted C₁-C₆ alkyl;
R⁵ is hydrogen or substituted or unsubstituted C₁-C₆ alkyl;
each of R⁶ is independently substituted or unsubstituted C₃-C₂₂ alkyl or substituted or unsubstituted C₃-C₂₂ alkenyl;
each of R⁷ and R⁸ is independently hydrogen or substituted or unsubstituted C₁-C₆ alkyl, or R⁷ and R⁸ taken together with the nitrogen to which they are attached form a substituted or unsubstituted C₂-C₆ heterocyclyl; and
p is an integer selected from 1 to 10.

In some embodiments of Formula (Ia), if the structure carries more than one asymmetric C-atom, each asymmetric C-atom independently represents racemic, chirally pure R and/or chirally pure S isomer, or a combination thereof.

### Variations of Formula (I) and (Ia)

In some embodiments, R¹ and R² in Formula (I) and Formula (Ia) is independently C₃-C₂₂ alkyl, C₃-C₂₂ alkenyl, —C₂-C₁₀ alkylene-L-R⁶, or

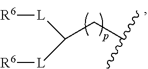

wherein each of the alkyl, alkylene, alkenyl, and cycloalkyl is independently substituted or unsubstituted. In some embodiments, R¹ and R² in Formula (I) and Formula (Ia) is independently C₁₀-C₂₀ alkyl, C₁₀-C₂₀ alkenyl. —C₈-C₇ alkylene-L-R⁶, or

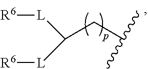

wherein each of the alkyl, alkylene, alkenyl, and cycloalkyl is independently substituted or unsubstituted. In some embodiments, R¹ in Formula (I) and Formula (Ia) is

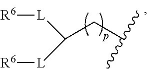

In some embodiments, each of L in Formula (I) and Formula (Ia) is independently O, S, —C₁-C₁₀ alkylene-O—, —C₁-C₁₀ alkylene-C(═O)O—, —C₁-C₁₀ alkylene-OC(═O)—, or a bond, wherein the alkylene is substituted or unsubstituted. In some embodiments, each of L in Formula (I) and Formula (Ia) is independently O, S, —C₁-C₃ alkylene-O—, —C₁-C₃ alkylene-C(═O)O—, —C₁-C₃ alkylene-OC(═O)—, or a bond, wherein the alkylene is substituted or unsubstituted. In some embodiments, each of L in Formula (I) and Formula (Ia) is independently O, S, —C₁-C₃ alkylene-O—, —C₁-C₃ alkylene-C(═O)O—, —C₁-C₃ alkylene-OC(═O)—, or a bond, wherein the alkylene is linear or branched unsubstituted alkylene.

In some embodiments, each of R⁶ in Formula (I) and Formula (Ia) is independently substituted or unsubstituted linear C₃-C₂₂ alkyl or substituted or unsubstituted linear C₃-C₂₂ alkenyl. In some embodiments, each of R⁶ in Formula (I) and Formula (Ia) is independently substituted or unsubstituted C₃-C₂₀ alkyl or substituted or unsubstituted C₃-C₂₀ alkenyl. In some embodiments, each of R⁶ in Formula (I) and Formula (Ia) is independently substituted or unsubstituted C₃-C₁₀ alkyl or substituted or unsubstituted C₃-C₁₀ alkenyl. In some embodiments, each of R⁶ in Formula (I) and Formula (Ia) is independently substituted or unsubstituted C₃-C₁₀ alkyl. In some embodiments, each of R⁶ in Formula (I) and Formula (Ia) is independently substituted or unsubstituted linear C₃-C₁₀ alkyl. In some embodiments, each of R⁶ in Formula (I) and Formula (Ia) is independently substituted or unsubstituted n-pentyl, n-hexyl, n-heptyl, n-octyl, n-nonyl, n-decyl, n-undecyl, or n-dodecyl. In some embodiments, each of R⁶ in Formula (I) and Formula (Ia) is independently substituted or unsubstituted n-octyl. In some embodiments, each of R⁶ in Formula (I) and Formula (Ia) is n-octyl.

In some embodiments, each of L in Formula (I) and Formula (Ia) is independently-C(═O)O—, —OC(═O)—, —C₁-C₁₀ alkylene-O—, or O. In some embodiments, each of. L in Formula (I) and Formula (Ia) is O. In some embodiments, each of L in Formula (I) and Formula (Ia) is —C₁-C₃ alkylene-O—. In some embodiments, p in Formula (I) and Formula (Ia) is 1, 2, 3, 4, or 5. In some embodiments, p in Formula (I) and Formula (Ia) is 2.

In some embodiments, R¹ in Formula (I) and Formula (Ia) is

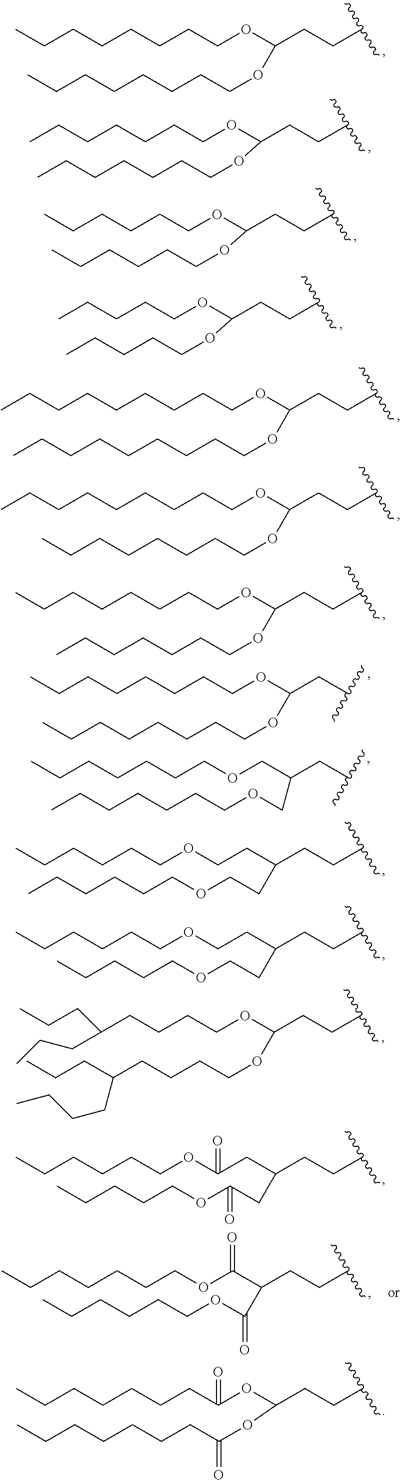

In some embodiments R¹ in Formula (I) and Formula (Ia) is R².

In some embodiments, each of R⁴ in Formula (I) and Formula (Ia) is independently H or substituted or unsubstituted C₁-C₄ alkyl. In some embodiments, each of. R⁴ in Formula (I) and Formula (Ia) is independently substituted or unsubstituted linear C₁-C₄ alkyl. In some embodiments, each of R⁴ in Formula (1) and Formula (Ia) is H. In some embodiments, each of R⁴ in Formula (I) and Formula (Ia) is independently H, —CH₃, —CH₂CH₃, —CH₂CH₂CH₃, or —CH(CH₃)₂. In some embodiments, each of R⁴ in Formula (I) and Formula (Ia) is independently H or —CH₃. In some embodiments, each of R⁴ in Formula (I) and Formula (Ia) is —CH₃.

In some embodiments, X in Formula (I) and Formula (Ia) is —C(═O)O— or —OC(═O))—. In some embodiments, X in Formula (I) and Formula (Ia) is —C(═O)NR⁴— or —NR⁴C(═O)—. In some embodiments, X in Formula (I) and Formula (Ia) is —C(═O)N(CH₃)—, —N(CH₃)C(═O)—, —C(═O) NH—, or —NHC(═O)—. In some embodiments, X in Formula (I) and Formula (Ia) is —C(═O)) NH—, —C(═O)N(CH₃)—, —OC(═O))—, —NHC(═O)—, —N(CH₃)C(═O))—, —C(═O)O—, —OC(═O)O—, —NHC(═O)O—, —N(CH₃)C(═O)O—, —OC(═O)) NH—, —OC(═O)N(CH₃)—, —NHC(═O) NH—, —N(CH₃)C(═O)) NH—, —NHC(═O)N(CH₃)—, —N(CH₃)C(═O)N(CH₃)—, NHC(═NH) NH—, —N(CH₃)C(═NH) NH—, —NHC(═NH)N(CH₃)—, —N(CH₃)C(═NH)N(CH₃)—, NHC(═NMe) NH—, —N(CH₃)C(═NMe) NH—, —NHC(═NMe)N(CH₃)—, or —N(CH₃)C(═NMe)N(CH₃)—.

In some embodiments. R² in Formula (I) and Formula (Ia) is C₃-C₂₂ alkyl, C₃-C₂₂ alkenyl, —C₂-C₁₀ alkylene-L-R⁶, or

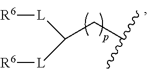

wherein each of the alkyl, alkylene, alkenyl, and cycloalkyl is independently substituted or unsubstituted. In some embodiments, R² in Formula (I) and Formula (Ia) is substituted or unsubstituted C₇-C₂₂ alkyl or substituted or unsubstituted C₃-C₂₂ alkenyl. In some embodiments, R² in Formula (I) and Formula (Ia) is substituted or unsubstituted linear C₇-C₂₂ alkyl or substituted or unsubstituted linear C₃-C₂₂ alkenyl. In some embodiments, R² in Formula (I) and Formula (Ia) is substituted or unsubstituted C₁₀-C₂₀ alkyl or substituted or unsubstituted C₁₀-C₂₀ alkenyl. In some embodiments, R² in Formula (I) and Formula (Ia) is unsubstituted C₁₀-C₂₀ alkyl. In some embodiments, R² in Formula (I) and Formula (Ia) is unsubstituted C₁₀-C₂₀ alkenyl. In some embodiments, R² in Formula (I) and Formula (Ia) is —C₂-C₁₀ alkylene-L—R⁶. In some embodiments, R² in Formula (I) and Formula (Ia) is —C₂-C₁₀ alkylene-C(═O)O—R⁶ or —C₂-C₁₀ alkylene-OC(═O)—R⁶.

In some embodiments, R² in Formula (I) and Formula (Ia) is

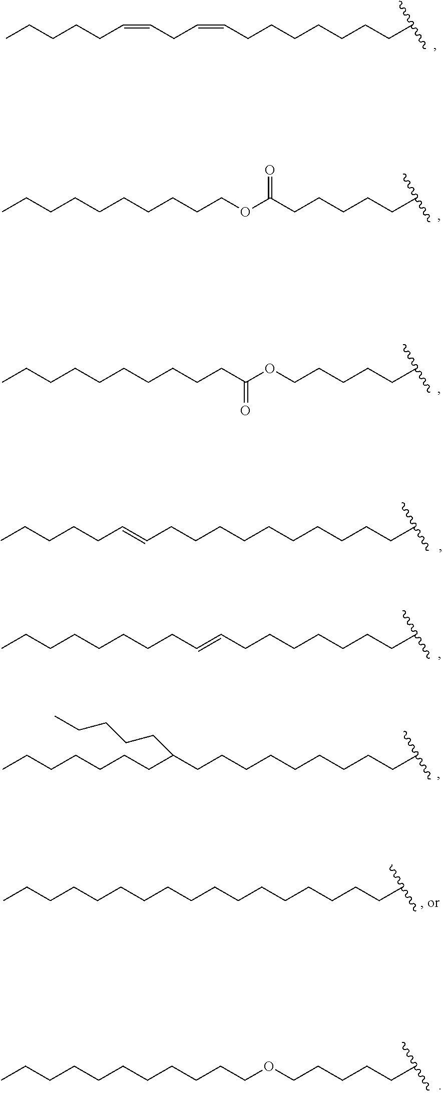

In some embodiments R¹ in Formula (I) and Formula (Ia) is R¹.

In some embodiments, Y in Formula (I) and Formula (Ia) is —C(═O)O— or —OC(═O)—. In some embodiments, Y in Formula (I) and Formula (Ia) is —C(═O)NR⁴— or —NR⁴C(═O)—. In some embodiments, Y in Formula (I) and Formula (Ia) is —C(═O)N(CH₃)—, —N(CH₃)C(═O)—, —C(═O)NH—, or —NHC(═O)—. In some embodiments, Y in Formula (I) and Formula (Ia) is —OC(═O)O—, —NR⁴C(═O)O—, —OC(═O)NR⁴—, or —NR⁴C(═O)NR⁴—. In some embodiments, Y in Formula (I) and Formula (Ia) is —OC(═O)O—, —NHC(═O)O—, —OC(═O) NH—, —NHC(═O) NH—, —N(CH₃)C(═O)O—, —OC(═O)N(CH₃)—, —N(CH₃)C(═O)N(CH₃)— or —N(CH₃)C(═O) NH—. In some embodiments, Y in Formula (I) and Formula (Ia) is —OC(═O)O—, —NHC(═O)₀—, —OC(═O) NH—, or —NHC(═O) NH—.

In some embodiments, R³ in Formula (I) and Formula (Ia) is —C₀-C₁₀ alkylene-NR⁷R⁸ or —C₀-C₁₀ alkylene-heterocycloalkyl, wherein the alkylene and heterocycloalkyl is independently substituted or unsubstituted. In some embodiments, R³ in Formula (I) and Formula (Ia) is —C₀-C₁₀ alkylene-NR⁷R⁸. In some embodiments, R³ in Formula (I) and Formula (Ia) is —C₁-C₆ alkylene-NR⁷R⁸. In some embodiments, R³ in Formula (I) and Formula (Ia) is —C₁-C₄ alkylene-NR⁷R⁸. In some embodiments, R³ in Formula (I) and Formula (Ia) is —C₁-alkylene-NR⁷R⁸. In some embodiments, R³ in Formula (I) and Formula (Ia) is —C₂—-alkylene-NR⁷R⁸. In some embodiments, R³ in Formula (I) and Formula (Ia) is —C₃-alkylene-NR⁷R⁸ In some embodiments, R³ in Formula (I) and Formula (Ia) is —C₄-alkylene-NR⁷R⁸. In some embodiments, R³ in Formula (I) and Formula (Ia) is —C₅-alkylene-NR⁷R⁸. In some embodiments, R³ in Formula (I) and Formula (Ia) is —C₀-C₁₀ alkylene-heterocycloalkyl. In some embodiments, R³ in Formula (I) and Formula (Ia) is —C₁-C₆ alkylene-heterocycloalkyl, wherein the heterocycloalkyl comprises 1 to 3 nitrogen and 0-2 oxygen. In some embodiments, R³ in Formula (I) and Formula (Ia) is —C₁-C₆ alkylene-heterocycloaryl.

In some embodiments, each of R⁷ and R⁸ in Formula (I) and Formula (Ia) is independently hydrogen or substituted or unsubstituted C₁-C₆ alkyl. In some embodiments, each of R⁷ and R⁸ is independently hydrogen or substituted or unsubstituted C₁-C₃ alkyl. In some embodiments, each of R⁷ and R⁸ is independently substituted or unsubstituted C₁-C₃ alkyl. In some embodiments, each of R⁷ and R⁸ is independently-CH₃, —CH₂CH₃, —CH₂CH₂CH₃, or —CH(CH₃)₂. In some embodiments, each of R and R⁸ is CH₃. In some embodiments, each of R⁷ and R⁸ is —CH₂CH₃.

In some embodiments, R⁷ and R⁸ in Formula (I) and Formula (Ia) taken together with the nitrogen to which they are attached form a substituted or unsubstituted C₂-C₆ heterocyclyl. In some embodiments, R⁷ and R⁸ taken together with the nitrogen to which they are attached form a substituted or unsubstituted C₂-C₆ heterocycloalkyl. In some embodiments, R⁷ and R⁸ taken together with the nitrogen to which they are attached form a substituted or unsubstituted 3-7 membered heterocycloalkyl.

In some embodiments, R³ in Formula (I) and Formula (Ia) is

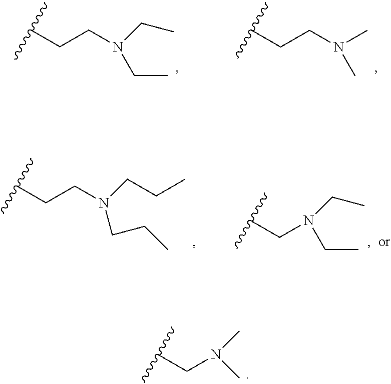

In some embodiments. R³ in Formula (I) and Formula (Ia) is

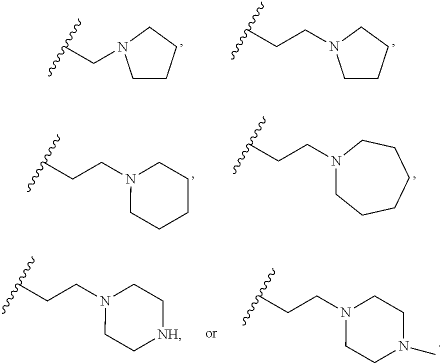

In some embodiments. R³ in Formula (1) and Formula (Ia) is

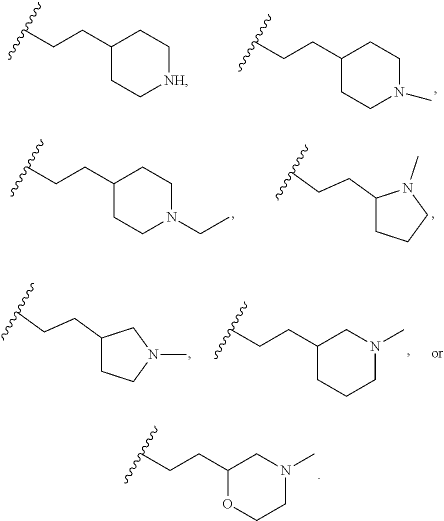

In some embodiments, Z in Formula (I) and Formula (Ia) is —C(═O)O— or —OC(═O)—.

In some embodiments, Z in Formula (I) and Formula (Ia) is —C(═O)NR⁴— or —NR⁴C(═O)—.

In some embodiments, Z in Formula (I) and Formula (Ia) is —C(═O)N(CH₃)—, —N(CH₃)C(═O)—, —C(═O) NH—, or —NHC(═O)—.

In some embodiments, Z in Formula (I) and Formula (Ia) is —OC(═O)O—, —NR⁴C(═O)O—, —OC(O)NR⁴—, or —NR⁴C(═O)NR⁴—.

In some embodiments, Z in Formula (I) and Formula (Ia) is —OC(═O)O—, —NHC(═O)O—, —OC(═O) NH—, —NHC(═O) NH—, —N(CH₃)C(═O)O—, —OC(═O)N(CH₃)—, —N(CH₃)C(═O)N(CH₃)—, —NHC(═O)N(CH₃)— or —N(CH₃)C(═O) NH—.

In some embodiments, Y in Formula (I) and Formula (Ia) is —OC(═O)O—, —NHC(═O)O—, —OC(═O) NH—, or —NHC(═O)NH—.

In some embodiments, R⁵ in Formula (I) and Formula (Ia) is hydrogen or substituted or unsubstituted C₁-C₃ alkyl.

In some embodiments, R⁵ in Formula (I) and Formula (Ia) is H, —CH₃, —CH—)CH₃, —CH₂CH₂CH₃, or —CH(CH₃)₂.

In some embodiments, R⁵ in Formula (I) and Formula (Ia) is H.

### Exemplary Lipids of WO2022140252

In another aspect, an amino lipid is according to any formula or structure, or a pharmaceutically acceptable salt or solvate thereof, as described in International Publication No. WO2022140252, which is hereby incorporated by reference in its entirety. In some embodiments, an amino lipid has a structure according to any of Formulae A′, A, I″, I′, I, II″, II′, II, III′, III, I″-a, I′-a, I-a, I″-a-i, I″-a-ii, I″-a-iii, I″-b, I′-b, I-b, I″-b-i, I″-b-ii, I″-b-iii, I″-c, I′-c, I-c, I″-c-i, I″-c-ii, I″-c-iii, I′-d, I-d, I′-d-i, II-a, II-a-i, III-a, and III-a-i of WO2022140252, or a pharmaceutically acceptable salt or solvate thereof. Exemplary amino lipids also include any of the lipids of Table 1 of WO2022140252, including any of the lipids represented by Examples 7-1 to 7-253 and Examples 8-1 to 8-106, or a pharmaceutically acceptable salt or solvate thereof.

In some embodiments, an amino lipid is according to Formula A′ of WO2022140252:

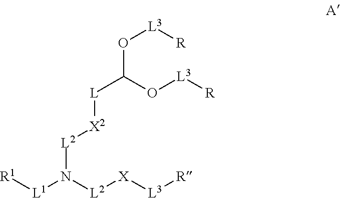

or its N-oxide, or a pharmaceutically acceptable salt thereof, wherein
L¹ is absent, C₁-6 alkylenyl, or C₂-6 heteroalkylenyl;
each L² is independently optionally substituted C₂₋₁₅ alkylenyl, or optionally substituted C₃₋₁₅ heteroalkylenyl;
L is C₁-10 alkylenyl, or C₂-10 heteroalkylenyl;
X² is —OC(O)—, —C(O)O—, or —OC(O)O—;
X is absent, —OC(O)—, —C(O)O—, or —OC(O)O—;
R″ is hydrogen,

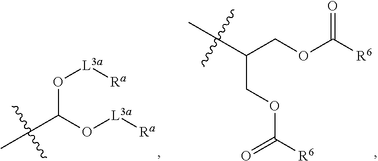

or an optionally substituted group selected from C₆-20 aliphatic, 3- to 12-membered cycloaliphatic, 7- to 12-membered bridged bicyclic comprising 0-4 heteroatoms independently selected from nitrogen, oxygen, or sulfur, 1-adamantyl, 2-adamantyl, sterolyl, and phenyl;
each of R and R^{a} is independently hydrogen, or an optionally substituted group selected from C₆-20 aliphatic, 3- to 12-membered cycloaliphatic, 7- to 12-membered bridged bicyclic comprising 0-4 heteroatoms independently selected from nitrogen, oxygen, or sulfur, 1-adamantyl, 2-adamantyl, sterolyl, and phenyl
each of L³ and L^{3a} is independently absent, optionally substituted C₁₋₁₀ alkylenyl, or optionally substituted C₂₋₁₀ heteroalkylenyl;
R¹ is hydrogen, optionally substituted phenyl, optionally substituted 3- to 7-membered cycloaliphatic, optionally substituted 3- to 7-membered heterocyclyl comprising 1-3 heteroatoms independently selected from nitrogen, oxygen, and sulfur, optionally substituted 5- to 6-membered monocyclic heteroaryl comprising 1-4 heteroatoms independently selected from nitrogen, oxygen, and sulfur, optionally substituted 8- to 10-membered bicyclic heteroaryl comprising 1-4 heteroatoms independently selected from nitrogen, oxygen, and sulfur, —OR², —C(O)OR², —C(O)SR², —OC(O)R², —OC(O)OR², —CN, —N(R²)₂, —C(O)N(R²)₂, —S(O)₂N(R²)₂, —NR²C(O)R², —OC(O)N(R²)₂, —N(R²)C(O)OR², —NR²S(O)₂R², —NR²C(O)N(R²)₂, —NR²C(S)N(R²)₂, —NR²C(NR²)N(R²)₂, —NR²C(CHR²)N(R²)₂, —N(OR²)C(O)R², —N(OR²) S(O)₂R², —N(OR²)C(O)OR², —N(OR²)C(O)N(R²)₂, —N(OR²)C(S)N(R²)₂, —N(OR²)C(NR²)N(R²)₂, —N(OR²)C(CHR²)N(R²)₂, —C(NR²)N(R²)₂, —C(NR²)R², —C(O)N(R²)OR², —C(R²)N(R²)₂C(O)OR², —CR² (R³)₂, —OP(O)(OR²)₂, or —P(O)(OR²)₂; or
R¹ is

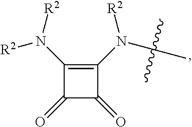

or a ring selected from 3- to 7-membered cycloaliphatic and 3- to 7-membered heterocyclyl comprising 1-3 heteroatoms independently selected from nitrogen, oxygen, and sulfur, wherein the cycloaliphatic or heterocyclyl ring is optionally substituted with 1-4 R² or R³ groups;
each R² is independently hydrogen, oxo, —CN, —NO₂, —OR⁴, —S(O)₂R⁴, —S(O)₂N(R⁴)₂, —(CH₂)ₙ—R⁴, or an optionally substituted group selected from C₁-6 aliphatic, phenyl, 3- to 7-membered cycloaliphatic, 5- to 6-membered monocyclic heteroaryl comprising 1-4 heteroatoms independently selected from nitrogen, oxygen, and sulfur, and 3- to 7-membered heterocyclyl comprising 1-3 heteroatoms independently selected from nitrogen, oxygen, and sulfur; or
   two occurrences of R², taken together with the atom(s) to which they are attached, form optionally substituted 4- to 7-membered heterocyclyl comprising 0-1 additional heteroatom selected from nitrogen, oxygen, and sulfur;
each R³ is independently —(CH₂)ₙ—R⁴; or two occurrences of R³, taken together with the atom(s) to which they are attached, form optionally substituted 5- to 6-membered heterocyclyl comprising 0-1 additional heteroatom selected from nitrogen, oxygen, and sulfur;
each R⁴ is independently hydrogen, —OR⁵, —N(R⁵)₂, —OC(O)R⁵, —OC(O)OR⁵, —CN, —C(O)N(R⁵)₂,
   —NR⁵C(O)R⁵, —OC(O)N(R⁵)₂, —N(R⁵)C(O)OR⁵, —NR⁵S(O)₂R⁵, —NR⁵C(O)N(R⁵)₂,
   —NR⁵C(S)N(R⁵)₂, —NR⁵C(NR⁵)N(R⁵)₂, or

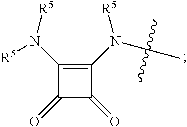

each R⁵ is independently hydrogen, or optionally substituted C₁₋₆ aliphatic; or two occurrences of R⁵, taken together with the atom(s) to which they are attached, form optionally substituted 4- to 7-membered heterocyclyl comprising 0-1 additional heteroatom selected from nitrogen, oxygen, and sulfur;
each R⁶ is independently C₄-12 aliphatic; and each n is independently 0 to 4.

In some embodiments, an amino lipid is according to Formula III-a of WO2022140252:

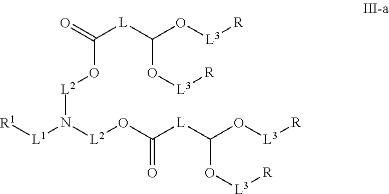

or its N-oxide, or a pharmaceutically acceptable salt thereof, wherein each of R, R¹, L, L¹, L², L³ is as defined therein for any of Formulae A′, A, III′, and III, and described in classes and subclasses above and herein, both singly and in combination. In embodiments of Formula III-a, each of R, R¹, L, L¹, L², L³ is as defined herein for Formula A′ above.

In some embodiments, an amino lipid is according to Formula III-a-i of WO2022140252:

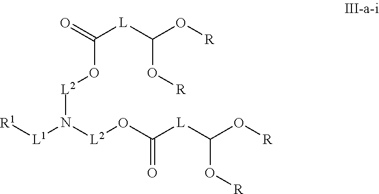

or its N-oxide, or a pharmaceutically acceptable salt thereof, wherein each of R, R¹, L, L¹, and L² is as defined therein for any of Formulae A′, A, III′, and III, and described in classes and subclasses above and herein, both singly and in combination. In embodiments of Formula III-a-i, each of R, R¹, L, L¹, and L² is as defined herein for Formula A′ above.

In some embodiments, an amino lipid is selected from any of the lipids described in

TABLE 1
of WO2022140252, or its N-oxide, or a pharmaceutically acceptable salt thereof. In
embodiments, an amino lipid is selected from the group consisting of:

| | |
|---|---|
| 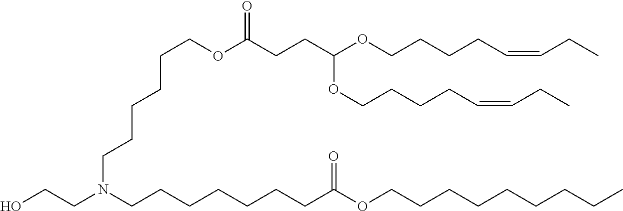 [SMILES: CC/C=C\CCCCOC(CCC(=O)OCCCCCCN(CCO)CCCCCCCC(=O)OCCCCCCCCC)OCCCC/C=C\CC] | 7-1 |
| 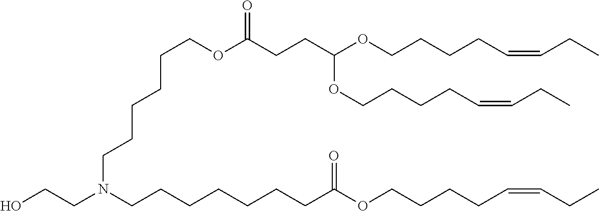 [SMILES: CC/C=C\CCCCOC(=O)CCCCCCCN(CCO)CCCCCCOC(=O)CCC(OCCCC/C=C\CC)OCCCC/C=C\CC] | 7-2 |
| 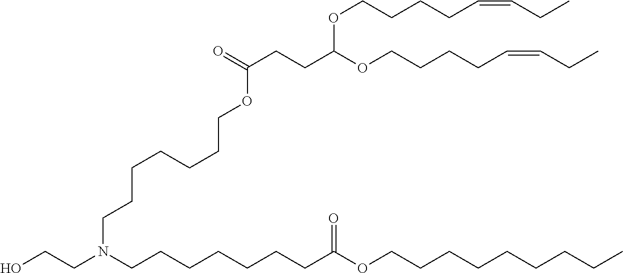 [SMILES: CC/C=C\CCCCOC(CCC(=O)OCCCCCCCN(CCO)CCCCCCCC(=O)OCCCCCCCCC)OCCCC/C=C\CC] | 7-19 |
| 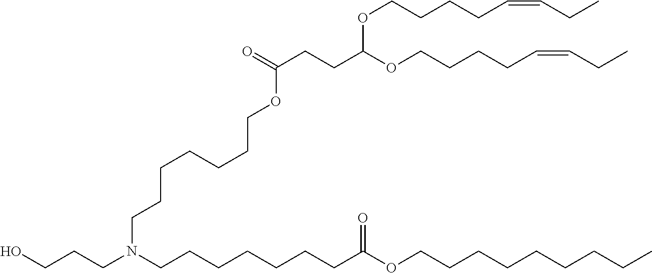 [SMILES: CC/C=C\CCCCOC(CCC(=O)OCCCCCCCN(CCCO)CCCCCCCC(=O)OCCCCCCCCC)OCCCC/C=C\CC] | 7-20 |
| 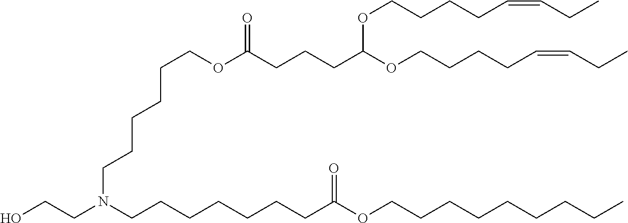 [SMILES: CC/C=C\CCCCOC(CCCC(=O)OCCCCCCN(CCO)CCCCCCCC(=O)OCCCCCCCCC)OCCCC/C=C\CC] | 7-22 |
| 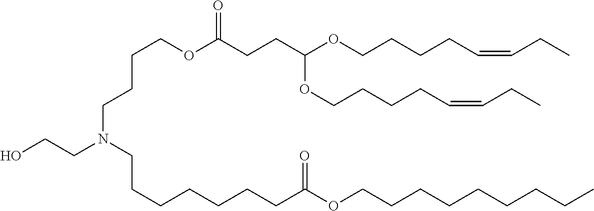 [SMILES: CC/C=C\CCCCOC(CCC(=O)OCCCCN(CCO)CCCCCCCC(=O)OCCCCCCCCC)OCCCC/C=C\CC] | 7-24 |
| 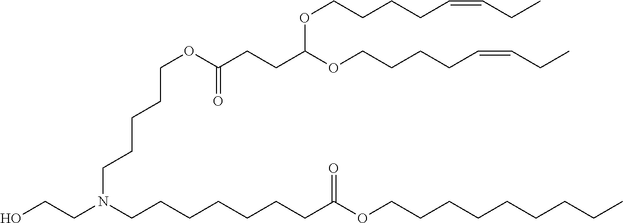 [SMILES: CC/C=C\CCCCOC(CCC(=O)OCCCCCN(CCO)CCCCCCCC(=O)OCCCCCCCCC)OCCCC/C=C\CC] | 7-25 |
| 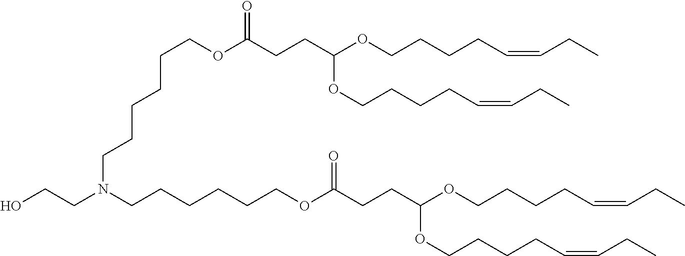 [SMILES: CC/C=C\CCCCOC(CCC(=O)OCCCCCCN(CCO)CCCCCCOC(=O)CCC(OCCCC/C=C\CC)OCCCC/C=C\CC)OCCCC/C=C\CC] | 8-1 |
| 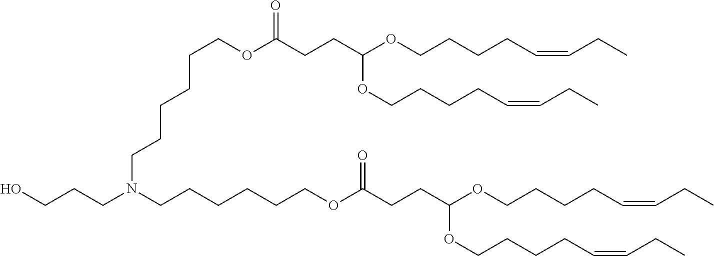 [SMILES: CC/C=C\CCCCOC(CCC(=O)OCCCCCCN(CCCO)CCCCCCOC(=O)CCC(OCCCC/C=C\CC)OCCCC/C=C\CC)OCCCC/C=C\CC] | 8-2 |
| 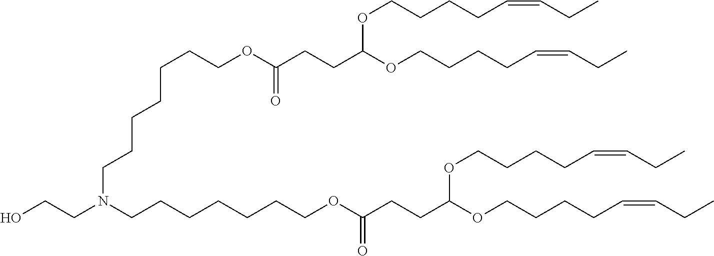 [SMILES: CC/C=C\CCCCOC(CCC(=O)OCCCCCCCN(CCO)CCCCCCCOC(=O)CCC(OCCCC/C=C\CC)OCCCC/C=C\CC)OCCCC/C=C\CC] | 8-3 |
| 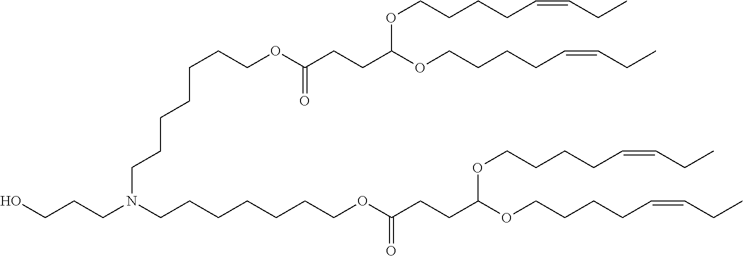 [SMILES: CC/C=C\CCCCOC(CCC(=O)OCCCCCCCN(CCCO)CCCCCCCOC(=O)CCC(OCCCC/C=C\CC)OCCCC/C=C\CC)OCCCC/C=C\CC] | 8-4 |
| 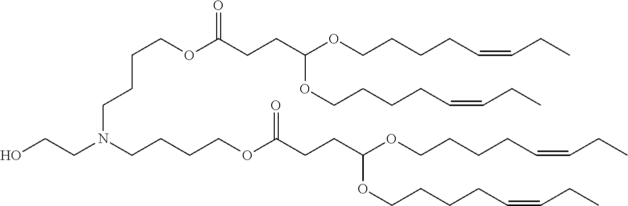 [SMILES: CC/C=C\CCCCOC(CCC(=O)OCCCCN(CCO)CCCCOC(=O)CCC(OCCCC/C=C\CC)OCCCC/C=C\CC)OCCCC/C=C\CC] | 8-5 |
| 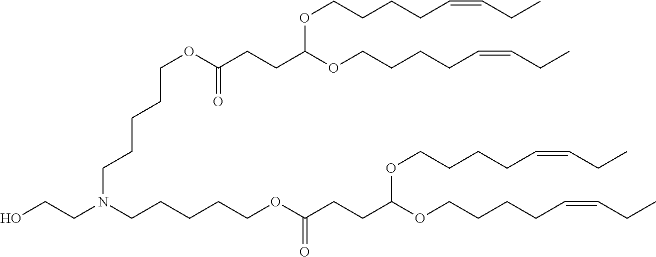 [SMILES: CC/C=C\CCCCOC(CCC(=O)OCCCCCN(CCO)CCCCCOC(=O)CCC(OCCCC/C=C\CC)OCCCC/C=C\CC)OCCCC/C=C\CC] | 8-6 |
| 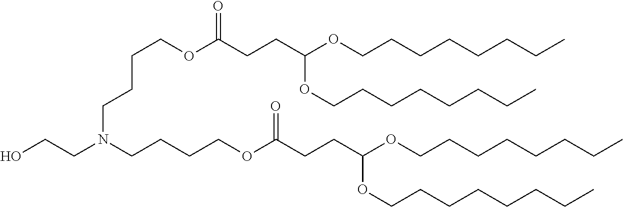 [SMILES: CCCCCCCCOC(CCC(=O)OCCCCN(CCO)CCCCOC(=O)CCC(OCCCCCCCC)OCCCCCCCC)OCCCCCCCC] | 8-7 |
| 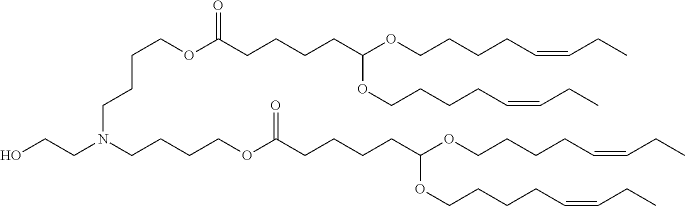 [SMILES: CC/C=C\CCCCOC(CCCCC(=O)OCCCCN(CCO)CCCCOC(=O)CCCCC(OCCCC/C=C\CC)OCCCC/C=C\CC)OCCCC/C=C\CC] | 8-13 |
| 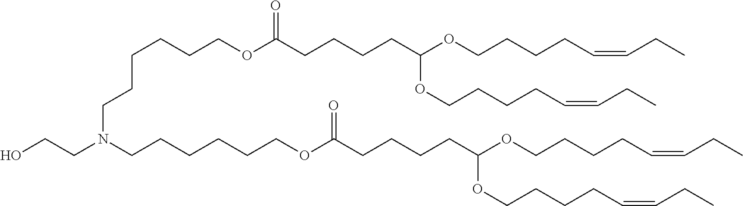 [SMILES: CC/C=C\CCCCOC(CCCCC(=O)OCCCCCCN(CCO)CCCCCCOC(=O)CCCCC(OCCCC/C=C\CC)OCCCC/C=C\CC)OCCCC/C=C\CC] | 8-14 |
| 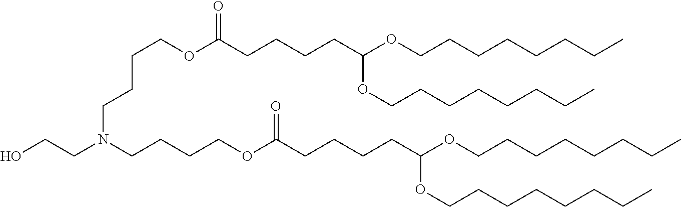 [SMILES: CCCCCCCCOC(CCCCC(=O)OCCCCN(CCO)CCCCOC(=O)CCCCC(OCCCCCCCC)OCCCCCCCC)OCCCCCCCC] | 8-17 |
| 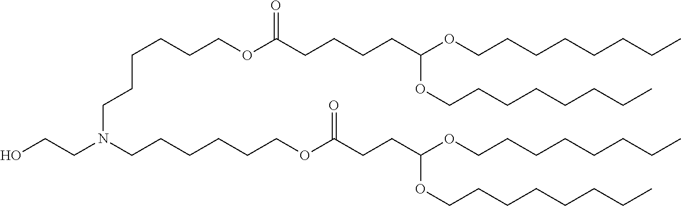 [SMILES: CCCCCCCCOC(CCCCC(=O)OCCCCCCN(CCO)CCCCCCOC(=O)CCC(OCCCCCCCC)OCCCCCCCC)OCCCCCCCC] | 8-18 |
| 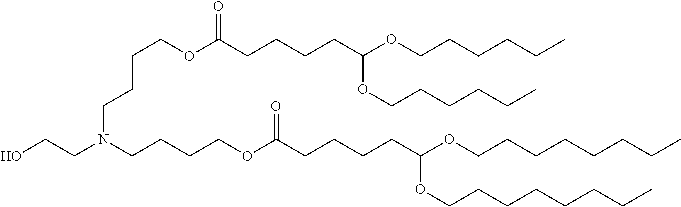 [SMILES: CCCCCCCCOC(CCCCC(=O)OCCCCN(CCO)CCCCOC(=O)CCCCC(OCCCCCC)OCCCCCC)OCCCCCCCC] | 8-19 |
| 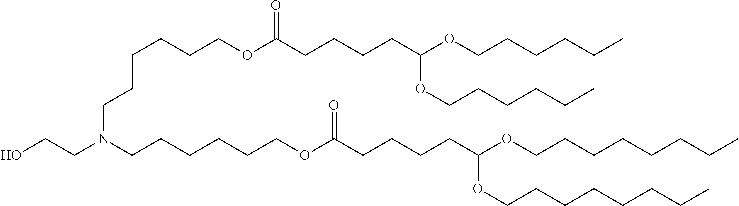 [SMILES: CCCCCCCCOC(CCCCC(=O)OCCCCCCN(CCO)CCCCCCOC(=O)CCCCC(OCCCCCC)OCCCCCC)OCCCCCCCC] | 8-20 |
| 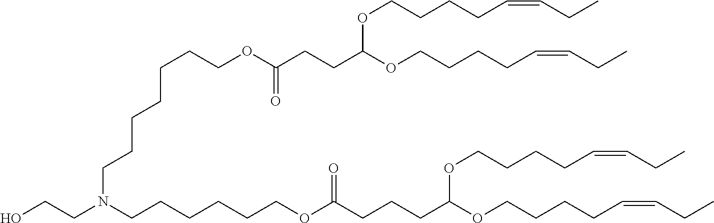 [SMILES: CC/C=C\CCCCOC(CCCC(=O)OCCCCCCN(CCO)CCCCCCCOC(=O)CCC(OCCCC/C=C\CC)OCCCC/C=C\CC)OCCCC/C=C\CC] | 8-55 |
| 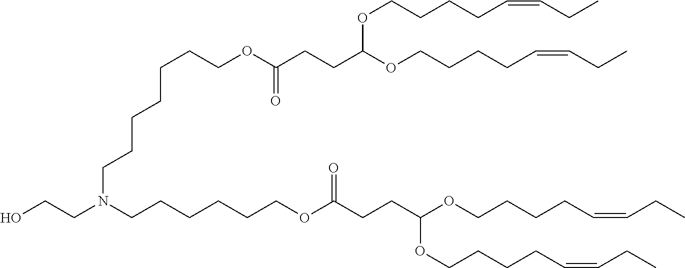 [SMILES: CC/C=C\CCCCOC(CCC(=O)OCCCCCCCN(CCO)CCCCCCOC(=O)CCC(OCCCC/C=C\CC)OCCCC/C=C\CC)OCCCC/C=C\CC] | 8-57 |
| 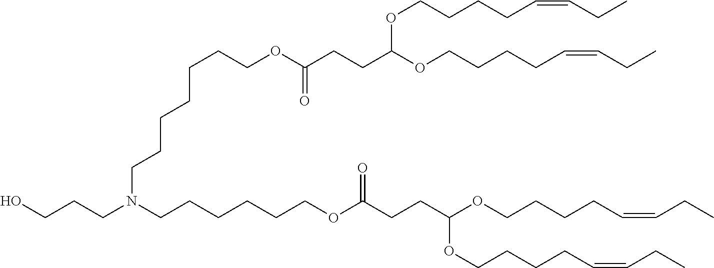 [SMILES: CC/C=C\CCCCOC(CCC(=O)OCCCCCCCN(CCCO)CCCCCCOC(=O)CCC(OCCCC/C=C\CC)OCCCC/C=C\CC)OCCCC/C=C\CC] | 8-58 |
| 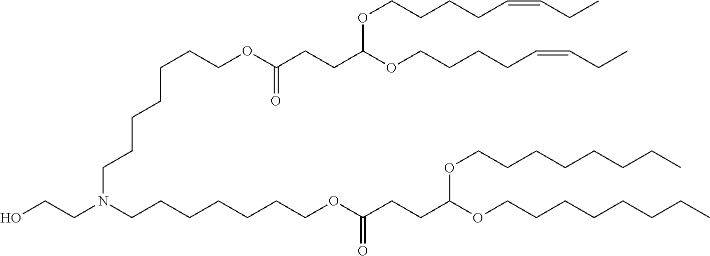 [SMILES: CC/C=C\CCCCOC(CCC(=O)OCCCCCCCN(CCO)CCCCCCCOC(=O)CCC(OCCCCCCCC)OCCCCCCCC)OCCCC/C=C\CC] | 8-59 |
| 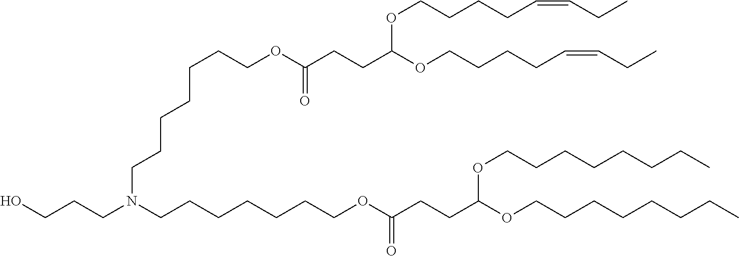 [SMILES: CC/C=C\CCCCOC(CCC(=O)OCCCCCCCN(CCCO)CCCCCCCOC(=O)CCC(OCCCCCCCC)OCCCCCCCC)OCCCC/C=C\CC] | 8-60 |
| 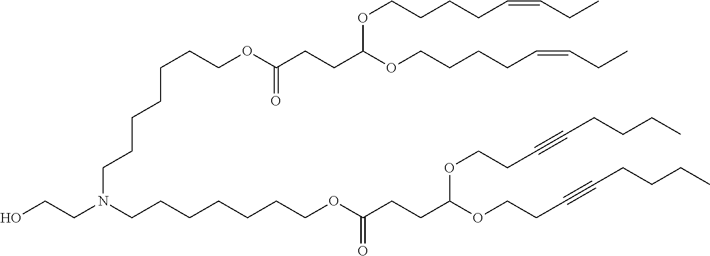 [SMILES: CC/C=C\CCCCOC(CCC(=O)OCCCCCCCN(CCO)CCCCCCCOC(=O)CCC(OCCC#CCCCC)OCCC#CCCCC)OCCCC/C=C\CC] | 8-61 |
| 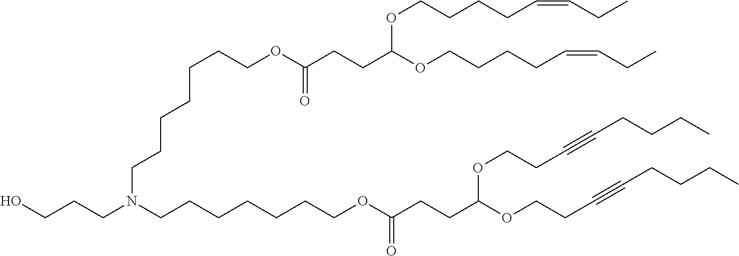 [SMILES: CC/C=C\CCCCOC(CCC(=O)OCCCCCCCN(CCCO)CCCCCCCOC(=O)CCC(OCCC#CCCCC)OCCC#CCCCC)OCCCC/C=C\CC] | 8-62 |
| 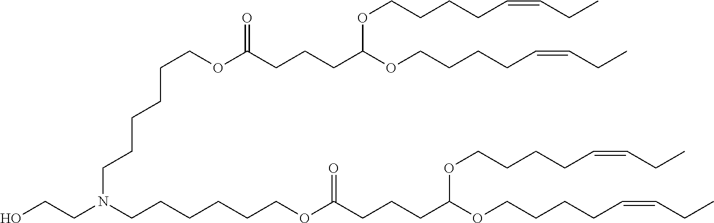 [SMILES: CC/C=C\CCCCOC(CCCC(=O)OCCCCCCN(CCO)CCCCCCOC(=O)CCCC(OCCCC/C=C\CC)OCCCC/C=C\CC)OCCCC/C=C\CC] | 8-63 |
| 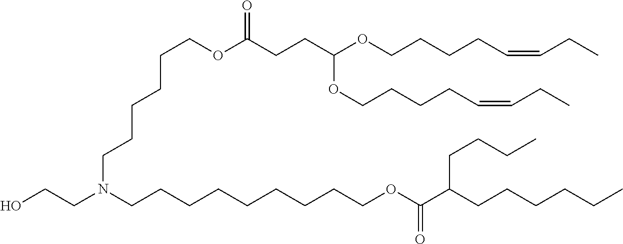 [SMILES: CC/C=C\CCCCOC(CCC(=O)OCCCCCCN(CCO)CCCCCCCCCOC(=O)C(CCCC)CCCCCC)OCCCC/C=C\CC] | 7-232 |
| 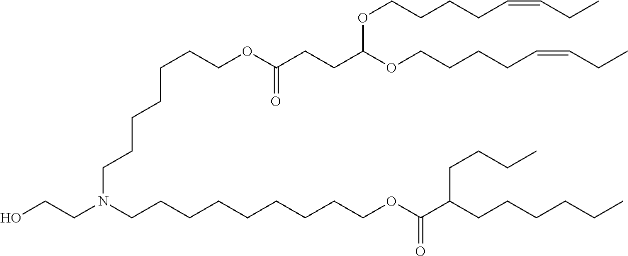 [SMILES: CC/C=C\CCCCOC(CCC(=O)OCCCCCCCN(CCO)CCCCCCCCCOC(=O)C(CCCC)CCCCCC)OCCCC/C=C\CC] | 7-233 |
| 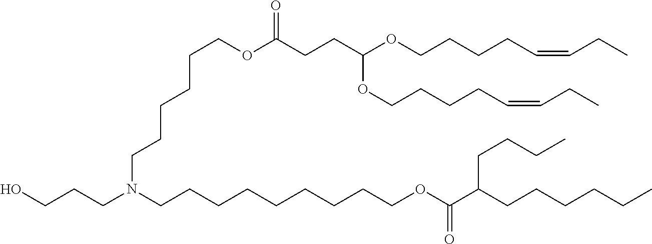 [SMILES: CC/C=C\CCCCOC(CCC(=O)OCCCCCCN(CCCO)CCCCCCCCCOC(=O)C(CCCC)CCCCCC)OCCCC/C=C\CC] | 7-234 |
| 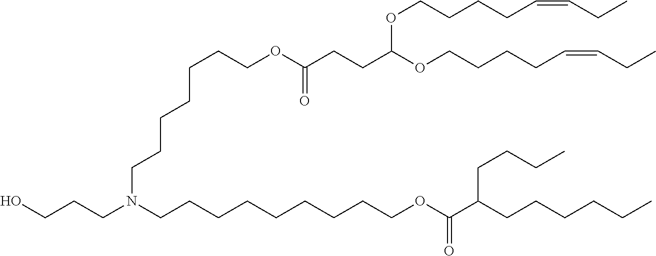 [SMILES: CC/C=C\CCCCOC(CCC(=O)OCCCCCCCN(CCCO)CCCCCCCCCOC(=O)C(CCCC)CCCCCC)OCCCC/C=C\CC] | 7-235 |
| 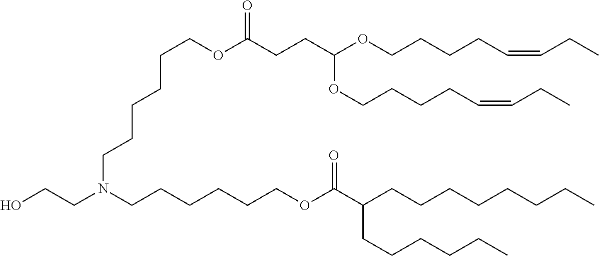 [SMILES: CC/C=C\CCCCOC(CCC(=O)OCCCCCCN(CCO)CCCCCCOC(=O)C(CCCCCC)CCCCCCCC)OCCCC/C=C\CC] | 7-236 |
| 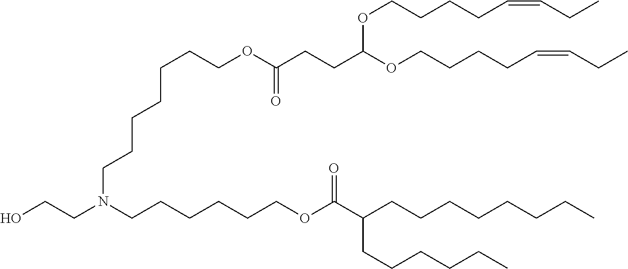 [SMILES: CC/C=C\CCCCOC(CCC(=O)OCCCCCCCN(CCO)CCCCCCOC(=O)C(CCCCCC)CCCCCCCC)OCCCC/C=C\CC] | 7-237 |
| 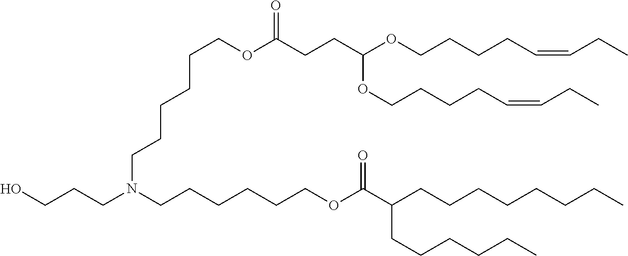 [SMILES: CC/C=C\CCCCOC(CCC(=O)OCCCCCCN(CCCO)CCCCCCOC(=O)C(CCCCCC)CCCCCCCC)OCCCC/C=C\CC] | 7-238 |
| 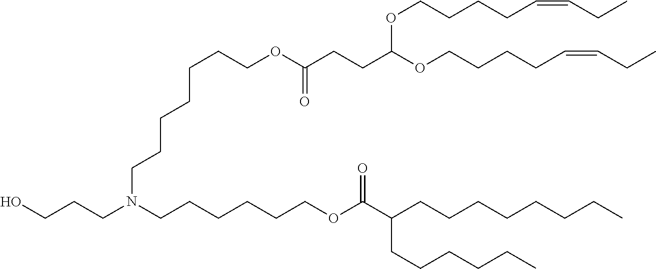 [SMILES: CC/C=C\CCCCOC(CCC(=O)OCCCCCCCN(CCCO)CCCCCCOC(=O)C(CCCCCC)CCCCCCCC)OCCCC/C=C\CC] | 7-239 |
| 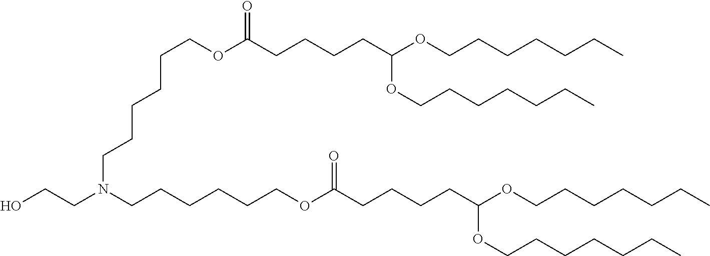 [SMILES: CCCCCCCOC(CCCCC(=O)OCCCCCCN(CCO)CCCCCCOC(=O)CCCCC(OCCCCCCC)OCCCCCCC)OCCCCCCC] | 8-67 |
| 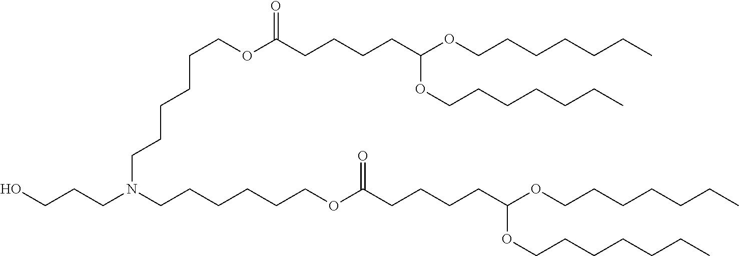 [SMILES: CCCCCCCOC(CCCCC(=O)OCCCCCCN(CCCO)CCCCCCOC(=O)CCCCC(OCCCCCCC)OCCCCCCC)OCCCCCCC] | 8-68 |
| 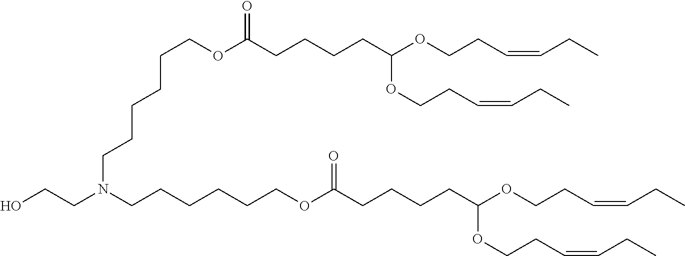 [SMILES: CC/C=C\CCOC(CCCCC(=O)OCCCCCCN(CCO)CCCCCCOC(=O)CCCCC(OCC/C=C\CC)OCC/C=C\CC)OCC/C=C\CC] | 8-69 |
| 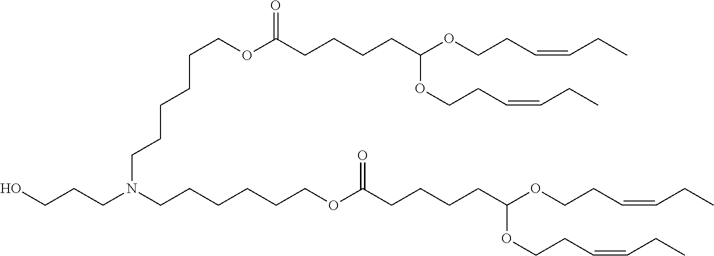 [SMILES: CC/C=C\CCOC(CCCCC(=O)OCCCCCCN(CCCO)CCCCCCOC(=O)CCCCC(OCC/C=C\CC)OCC/C=C\CC)OCC/C=C\CC] | 8-70 |
| 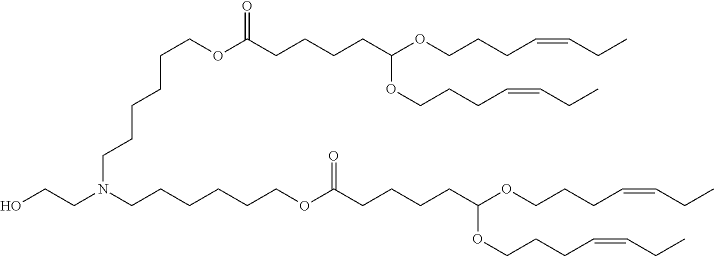 [SMILES: CC/C=C\CCCOC(CCCCC(=O)OCCCCCCN(CCO)CCCCCCOC(=O)CCCCC(OCCC/C=C\CC)OCCC/C=C\CC)OCCC/C=C\CC] | 8-71 |
| 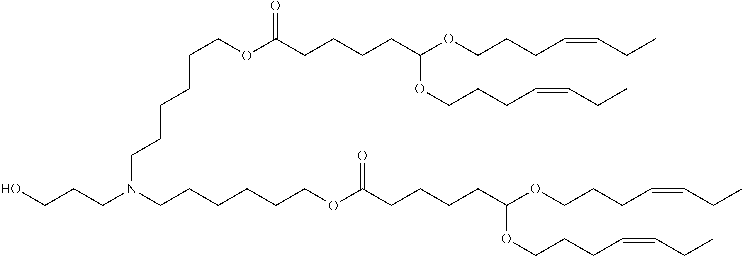 [SMILES: CC/C=C\CCCOC(CCCCC(=O)OCCCCCCN(CCCO)CCCCCCOC(=O)CCCCC(OCCC/C=C\CC)OCCC/C=C\CC)OCCC/C=C\CC] | 8-72 |
| 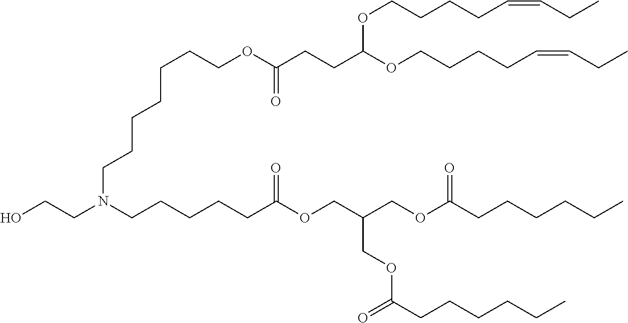 [SMILES: CC/C=C\CCCCOC(CCC(=O)OCCCCCCCN(CCO)CCCCCC(=O)OCC(COC(=O)CCCCCC)COC(=O)CCCCCC)OCCCC/C=C\CC] | 7-243 |
| 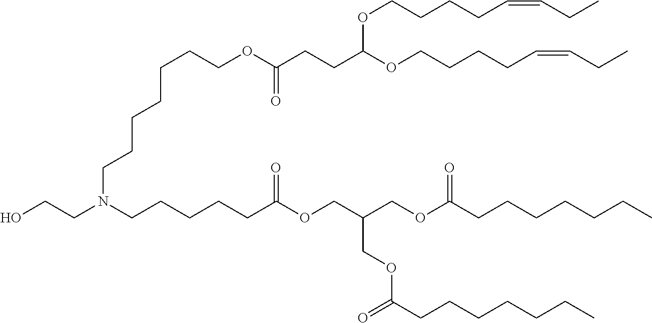 [SMILES: CC/C=C\CCCCOC(CCC(=O)OCCCCCCCN(CCO)CCCCCC(=O)OCC(COC(=O)CCCCCCC)COC(=O)CCCCCCC)OCCCC/C=C\CC] | 7-244 |
| 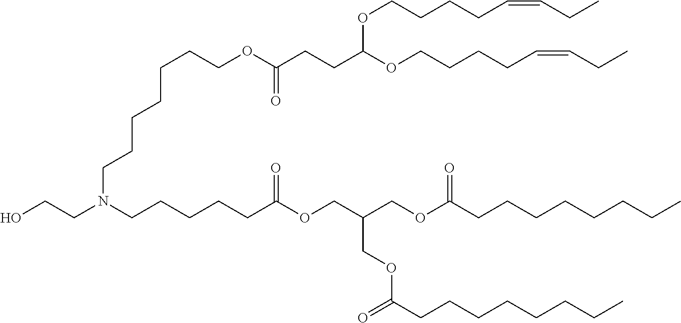 [SMILES: CC/C=C\CCCCOC(CCC(=O)OCCCCCCCN(CCO)CCCCCC(=O)OCC(COC(=O)CCCCCCCC)COC(=O)CCCCCCCC)OCCCC/C=C\CC] | 7-245 |
| 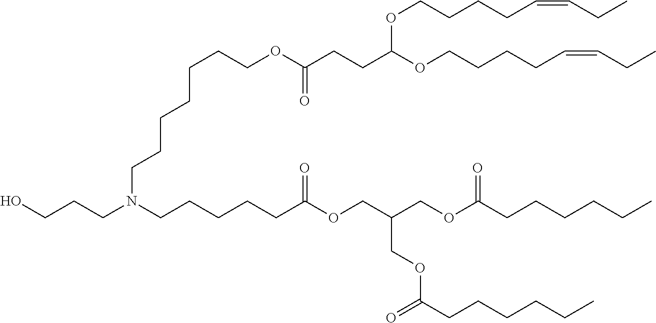 [SMILES: CC/C=C\CCCCOC(CCC(=O)OCCCCCCCN(CCCO)CCCCCC(=O)OCC(COC(=O)CCCCCC)COC(=O)CCCCCC)OCCCC/C=C\CC] | 7-246 |

In some embodiments, an amino lipid is Example 7-1, or a pharmaceutically acceptable salt thereof. In some embodiments, an amino lipid is Example 7-2, or a pharmaceutically acceptable salt thereof. In some embodiments, an amino lipid is Example 7-19, or a pharmaceutically acceptable salt thereof. In some embodiments, an amino lipid is Example 7-20, or a pharmaceutically acceptable salt thereof. In some embodiments, an amino lipid is Example 7-22, or a pharmaceutically acceptable salt thereof. In some embodiments, an amino lipid is Example 7-24, or a pharmaceutically acceptable salt thereof. In some embodiments, an amino lipid is Example 7-25, or a pharmaceutically acceptable salt thereof. In some embodiments, an amino lipid is Example 8-1, or a pharmaceutically acceptable salt thereof. In some embodiments, an amino lipid is Example 8-2, or a pharmaceutically acceptable salt thereof. In some embodiments, an amino lipid is Example 8-3, or a pharmaceutically acceptable salt thereof. In some embodiments, an amino lipid is Example 8-4, or a pharmaceutically acceptable salt thereof. In some embodiments, an amino lipid is Example 8-5, or a pharmaceutically acceptable salt thereof. In some embodiments, an amino lipid is Example 8-13, or a pharmaceutically acceptable salt thereof. In some embodiments, an amino lipid is Example 8-14, or a pharmaceutically acceptable salt thereof. In some embodiments, an amino lipid is Example 8-17, or a pharmaceutically acceptable salt thereof. In some embodiments, an amino lipid is Example 8-18, or a pharmaceutically acceptable salt thereof. In some embodiments, an amino lipid is Example 8-19, or a pharmaceutically acceptable salt thereof. In some embodiments, an amino lipid is Example 8-20, or a pharmaceutically acceptable salt thereof. In some embodiments, an amino lipid is Example 8-55, or a pharmaceutically acceptable salt thereof. In some embodiments, an amino lipid is Example 8-57, or a pharmaceutically acceptable salt thereof. In some embodiments, an amino lipid is Example 8-58, or a pharmaceutically acceptable salt thereof. In some embodiments, an amino lipid is Example 8-59, or a pharmaceutically acceptable salt thereof. In some embodiments, an amino lipid is Example 8-60, or a pharmaceutically acceptable salt thereof. In some embodiments, an amino lipid is Example 8-61, or a pharmaceutically acceptable salt thereof. In some embodiments, an amino lipid is Example 8-62, or a pharmaceutically acceptable salt thereof. In some embodiments, an amino lipid is Example 8-63, or a pharmaceutically acceptable salt thereof. In some embodiments, an amino lipid is Example 7-232, or a pharmaceutically acceptable salt thereof. In some embodiments, an amino lipid is Example 7-233, or a pharmaceutically acceptable salt thereof. In some embodiments, an amino lipid is Example 7-234, or a pharmaceutically acceptable salt thereof. In some embodiments, an amino lipid is Example 7-235, or a pharmaceutically acceptable salt thereof. In some embodiments, an amino lipid is Example 7-236, or a pharmaceutically acceptable salt thereof. In some embodiments, an amino lipid is Example 7-237, or a pharmaceutically acceptable salt thereof. In some embodiments, an amino lipid is Example 7-238, or a pharmaceutically acceptable salt thereof. In some embodiments, an amino lipid is Example 7-239, or a pharmaceutically acceptable salt thereof. In some embodiments, an amino lipid is Example 8-67, or a pharmaceutically acceptable salt thereof. In some embodiments, an amino lipid is Example 8-68, or a pharmaceutically acceptable salt thereof. In some embodiments, an amino lipid is Example 8-69, or a pharmaceutically acceptable salt thereof. In some embodiments, an amino lipid is Example 8-70, or a pharmaceutically acceptable salt thereof. In some embodiments, an amino lipid is Example 8-71, or a pharmaceutically acceptable salt thereof. In some embodiments, an amino lipid is Example 8-72, or a pharmaceutically acceptable salt thereof. In some embodiments, an amino lipid is Example 7-243, or a pharmaceutically acceptable salt thereof. In some embodiments, an amino lipid is Example 7-244, or a pharmaceutically acceptable salt thereof. In some embodiments, an amino lipid is Example 7-245, or a pharmaceutically acceptable salt thereof. In some embodiments, an amino lipid is Example 7-246, or a pharmaceutically acceptable salt thereof.

### Exemplary Lipids of WO2022159472

In another aspect, an amino lipid is according to any formula or structure, or a pharmaceutically acceptable salt or solvate thereof, as described in International Publication No. WO2022159472, which is hereby incorporated by reference in its entirety. In some embodiments, an amino lipid has a structure according to any of Formulae I, II, III, IIIA, IIIB, IIIC, IV, V, VA, VI, VIA, VII, and VIIA of WO2022159472, or a pharmaceutically acceptable salt or solvate thereof. Exemplary amino lipids also include any of the lipids of Table 1 of WO2022159472, including any of the lipids represented by Examples 4-1 to 4-86, or a pharmaceutically acceptable salt or solvate thereof.

In some embodiments, an amino lipid is according to Formula I of WO2022159472:

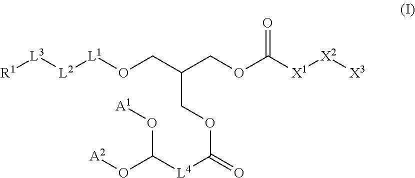

or a pharmaceutically acceptable salt thereof, wherein:
L¹ is a covalent bond, —C(O)—, or —OC(O)—;
L² is a covalent bond, an optionally substituted bivalent saturated or unsaturated, straight or branched C₁-C₁₂ hydrocarbon chain, or

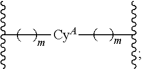

Cy^{A} is an optionally substituted ring selected from phenylene and 3- to 7-membered saturated or partially unsaturated carbocyclene;
each m is independently 0, 1, or 2;
L³ is a covalent bond, —C(O)—, —C(O)O—, —OC(O)—, —O—, or —OC(O)O—;
R¹ is

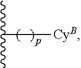

an optionally substituted saturated or unsaturated, straight or branched C₁-C₂₀ hydrocarbon chain wherein 1-3 methylene units are optionally and independently replaced with —O— or —NR—, or

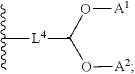

Cy^{B} is an optionally substituted ring selected from 3- to 12-membered saturated or partially unsaturated carbocyclyl, 1-adamantyl, 2-adamantyl,

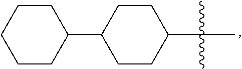 [SMILES: *C1CCC(C2CCCCC2)CC1]

sterolyl, and phenyl;
p is 0, 1, 2, or 3;
each L⁴ is independently a bivalent saturated or unsaturated, straight or branched C₁-C₆ hydrocarbon chain;
each A¹ and A² is independently an optionally substituted C₁-C₂₀ aliphatic or —L⁵-R⁵;
   or A¹ and A², together with their intervening atoms, may form an optionally substituted ring:

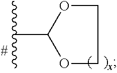

where
   x is selected from 1 or 2; and
   #represents the point of attachment to L⁴;
each L⁵ is independently a bivalent saturated or unsaturated, straight or branched C₁-C₂₀ hydrocarbon chain, wherein 1-3 methylene units are optionally and independently replaced with —O— or —NR—;
each R⁵ is independently an optionally substituted group selected from a 5- to 10-membered aryl ring or a 3- to 8-membered carbocyclic ring;
X¹ is a covalent bond, —O—, or —NR—;
X² is a covalent bond or an optionally substituted, bivalent saturated or unsaturated, straight or branched, C₁-C₁₂ hydrocarbon chain, wherein 1-3 methylene units are optionally and independently replaced with —O—, —NR—, or —Cy^{C}—;
Cy^{C} is an optionally substituted ring selected from 3- to 7-membered saturated or partially unsaturated carbocyclene, phenylene, 3- to 7-membered saturated or partially unsaturated heterocyclene having 1-3 heteroatoms independently selected from nitrogen, oxygen, and sulfur, and 5- to 6-membered heteroarylene having 1-3 heteroatoms independently selected from nitrogen, oxygen, and sulfur;
X³ is hydrogen or an optionally substituted ring selected from 3- to 7-membered saturated or partially unsaturated carbocyclyl, phenyl, 3- to 7-membered saturated or partially unsaturated heterocyclyl having 1-3 heteroatoms independently selected from nitrogen, oxygen, and sulfur, or 5- to 6-membered heteroaryl having 1-3 heteroatoms independently selected from nitrogen, oxygen, and sulfur; and
each R is independently hydrogen or an optionally substituted C₁-C₆ aliphatic group; provided that when L³ is a covalent bond, then R¹ must be

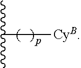

In some embodiments, an amino lipid is according to Formula VI of WO2022159472:

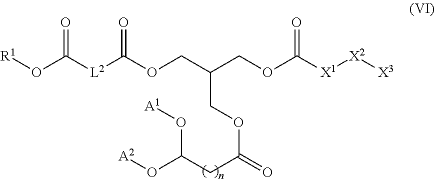

or a pharmaceutically acceptable salt thereof, wherein n is 1, 2, 3 or 4, and L², R¹, A¹, A², X¹,

X², and X³ are as defined therein for Formula I and also described in classes and subclasses therein, both singly and in combination. In embodiments, L², R¹, A¹, A², X¹, X², and X³ are as defined herein for Formula I above.

In some embodiments, an amino lipid is according to Formula VIA of WO2022159472:

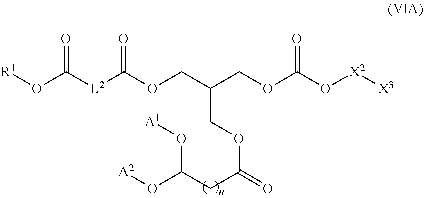

or a pharmaceutically acceptable salt thereof, wherein n is 1, 2, 3 or 4, and L², R¹, A¹, A², X², and X³ are as defined therein for Formula I and also described in classes and subclasses therein, both singly and in combination. In embodiments, L², R¹, A¹, A², X², and X³ are as defined herein for Formula I above.

In some embodiments, an amino lipid is selected from any of the lipids described in Table 1 of WO2022159472, or a pharmaceutically acceptable salt thereof. In embodiments, an amino lipid is selected from the group consisting of:

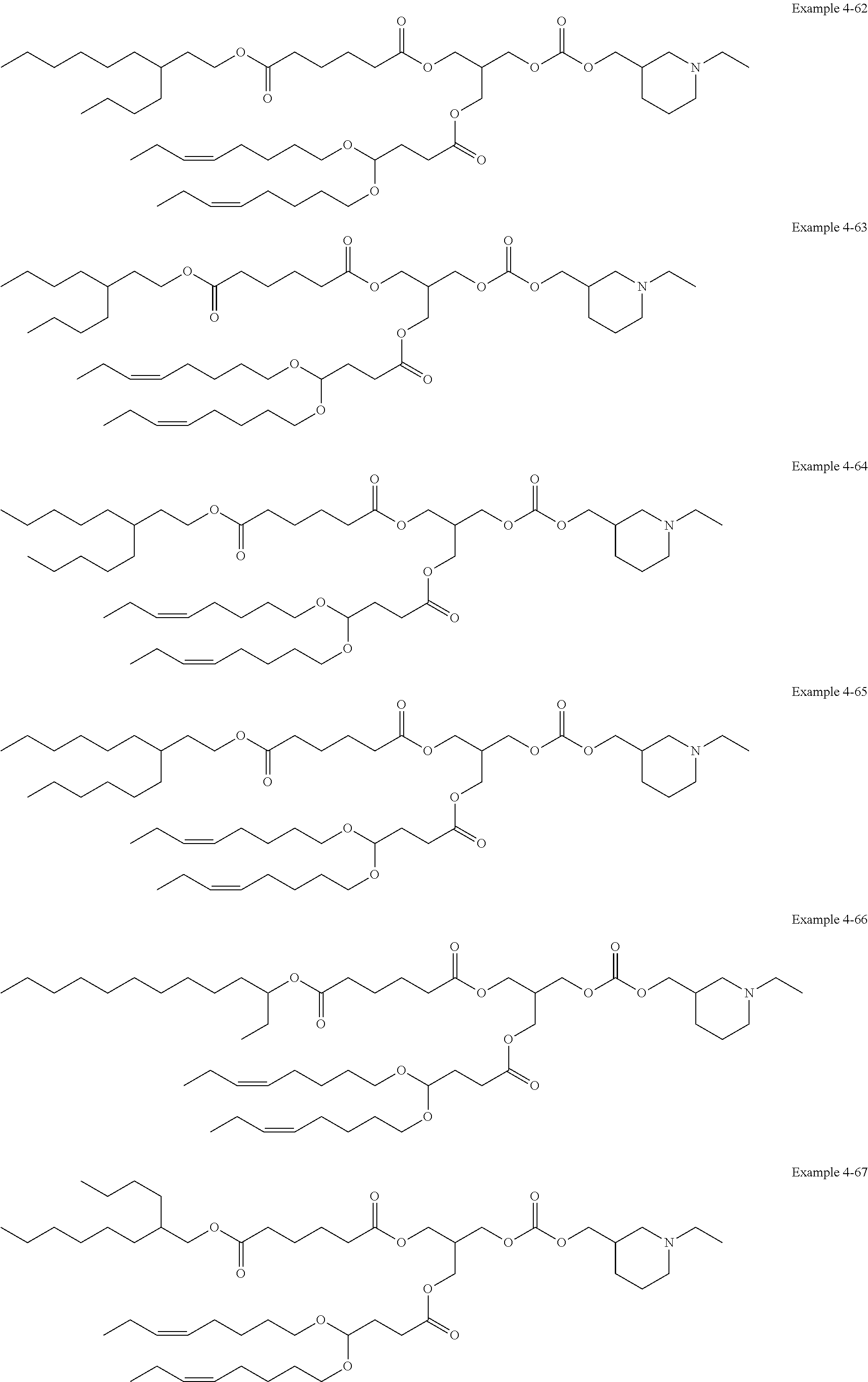

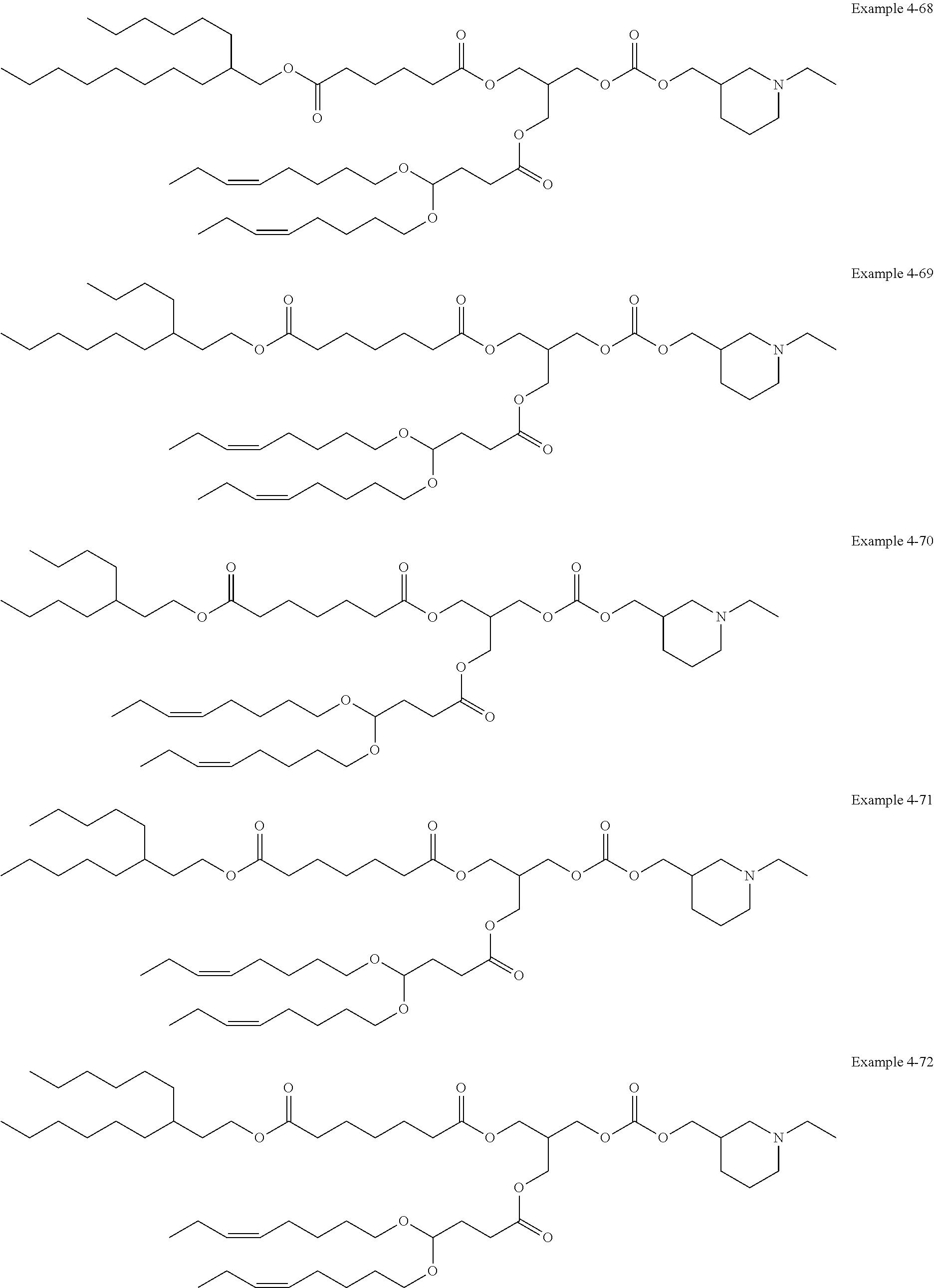

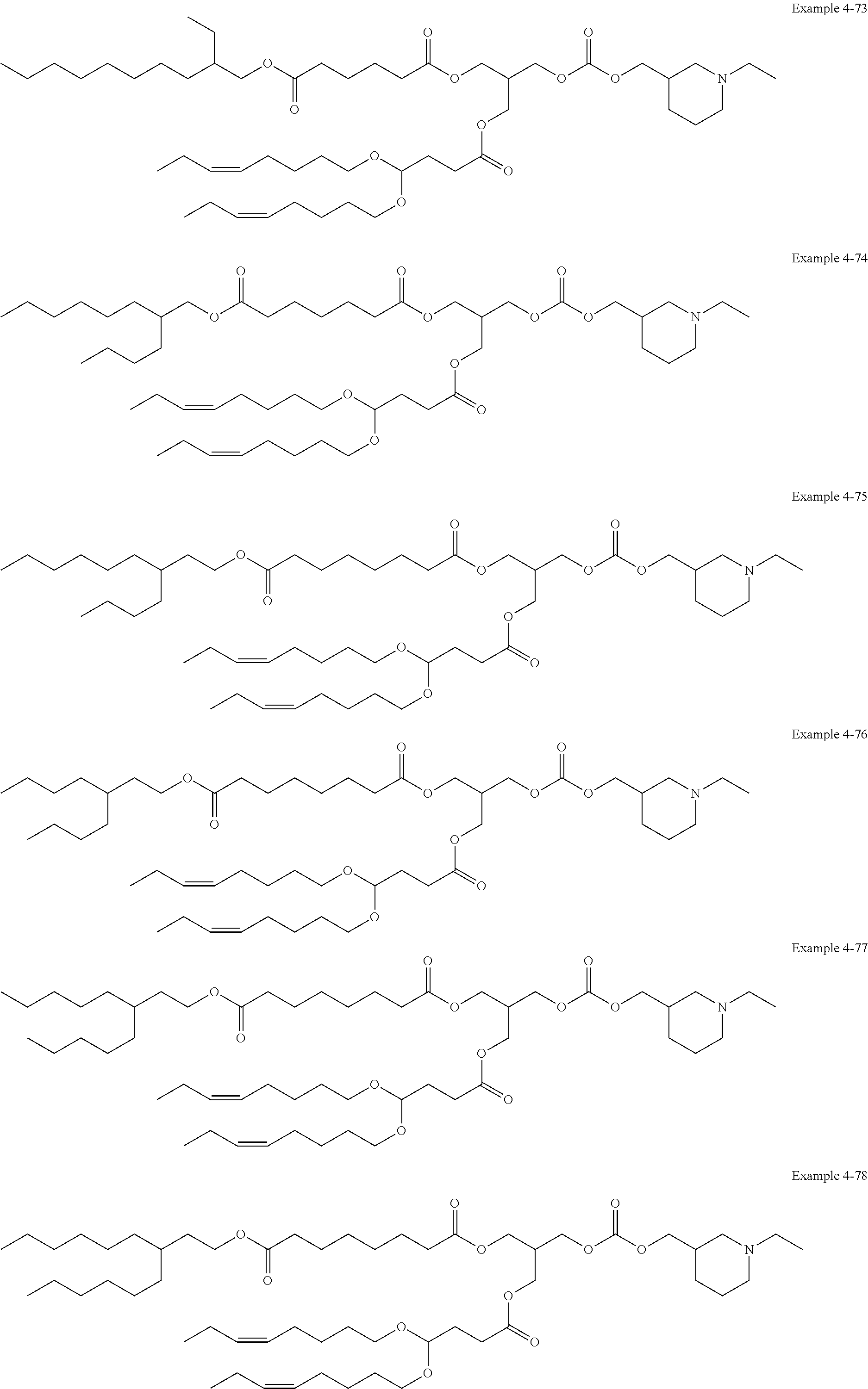

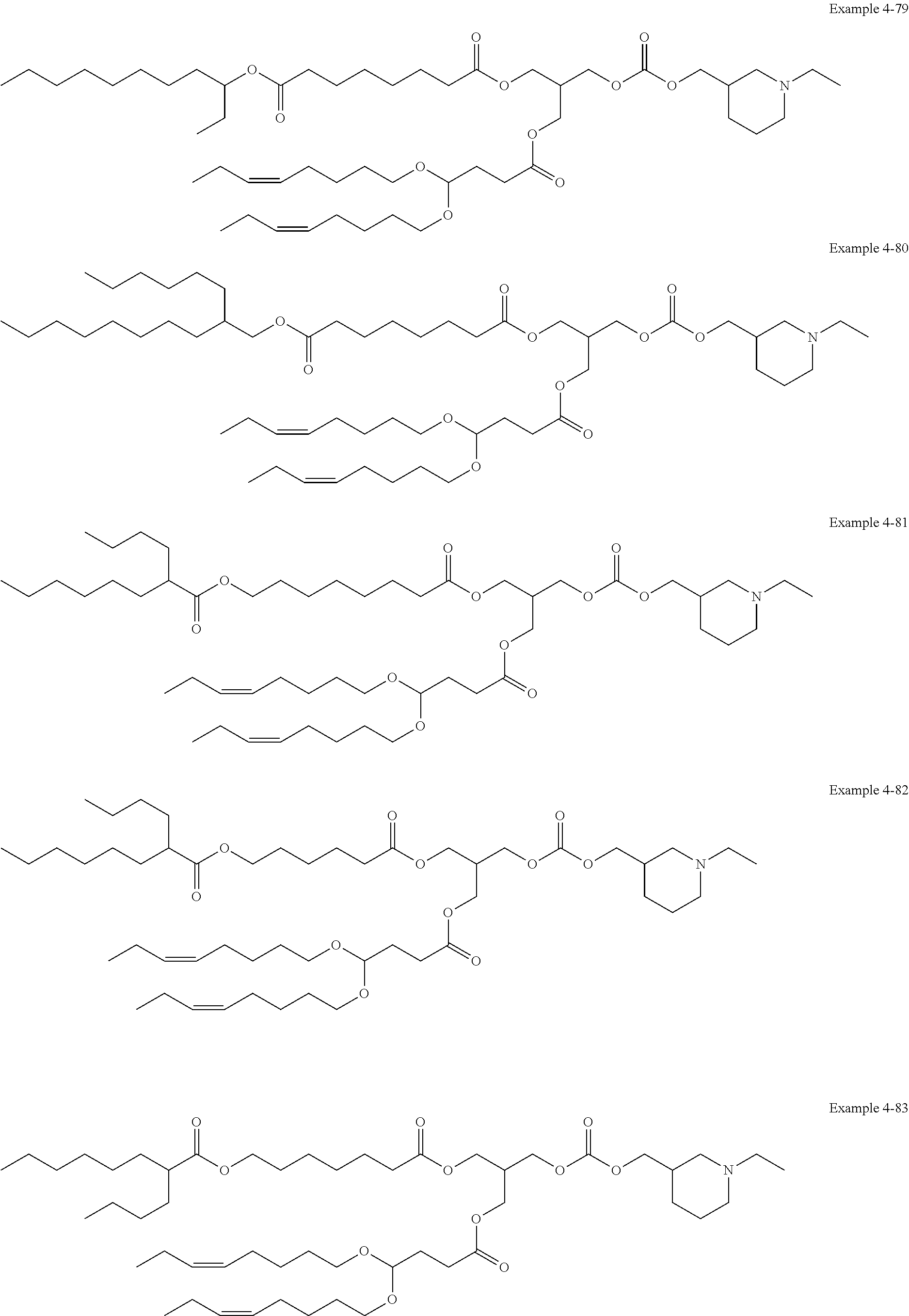

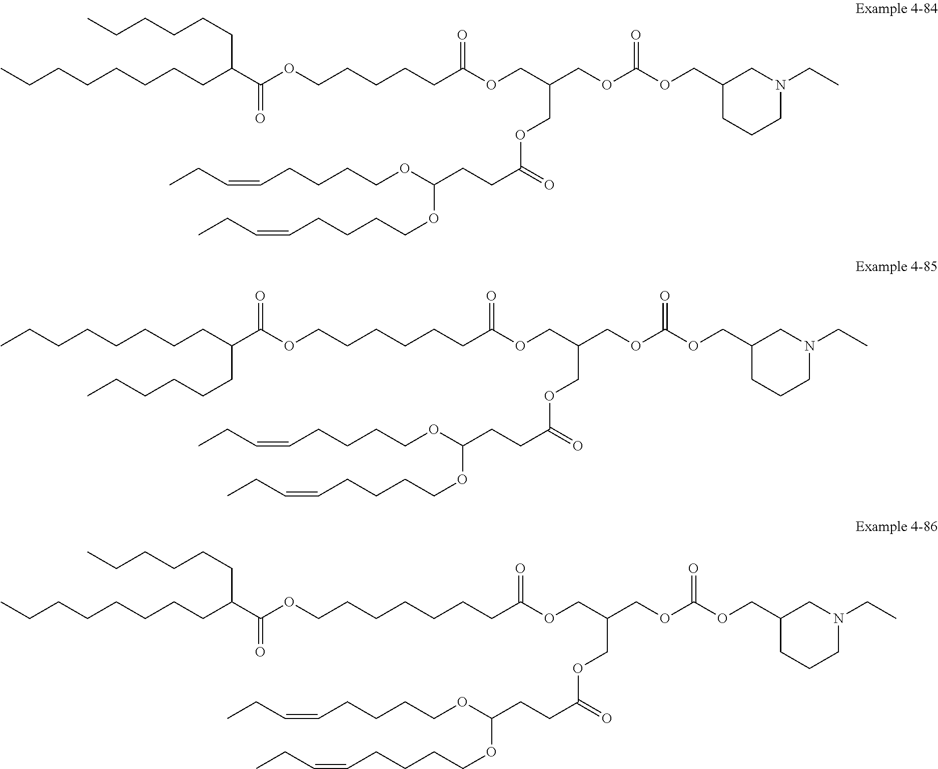

In some embodiments, an amino lipid is Example 4-62, or a pharmaceutically acceptable salt thereof. In some embodiments, an amino lipid is Example 4-63, or a pharmaceutically acceptable salt thereof. In some embodiments, an amino lipid is Example 4-64, or a pharmaceutically acceptable salt thereof. In some embodiments, an amino lipid is Example 4-65, or a pharmaceutically acceptable salt thereof. In some embodiments, an amino lipid is Example 4-66, or a pharmaceutically acceptable salt thereof. In some embodiments, an amino lipid is Example 4-67, or a pharmaceutically acceptable salt thereof. In some embodiments, an amino lipid is Example 4-68, or a pharmaceutically acceptable salt thereof. In some embodiments, an amino lipid is Example 4-69, or a pharmaceutically acceptable salt thereof. In some embodiments, an amino lipid is Example 4-70, or a pharmaceutically acceptable salt thereof. In some embodiments, an amino lipid is Example 4-71, or a pharmaceutically acceptable salt thereof. In some embodiments, an amino lipid is Example 4-72, or a pharmaceutically acceptable salt thereof. In some embodiments, an amino lipid is Example 4-73, or a pharmaceutically acceptable salt thereof. In some embodiments, an amino lipid is Example 4-74, or a pharmaceutically acceptable salt thereof. In some embodiments, an amino lipid is Example 4-75, or a pharmaceutically acceptable salt thereof. In some embodiments, an amino lipid is Example 4-76, or a pharmaceutically acceptable salt thereof. In some embodiments, an amino lipid is Example 4-77, or a pharmaceutically acceptable salt thereof. In some embodiments, an amino lipid is Example 4-78, or a pharmaceutically acceptable salt thereof. In some embodiments, an amino lipid is Example 4-79, or a pharmaceutically acceptable salt thereof. In some embodiments, an amino lipid is Example 4-80, or a pharmaceutically acceptable salt thereof. In some embodiments, an amino lipid is Example 4-81, or a pharmaceutically acceptable salt thereof. In some embodiments, an amino lipid is Example 4-82, or a pharmaceutically acceptable salt thereof. In some embodiments, an amino lipid is Example 4-83, or a pharmaceutically acceptable salt thereof. In some embodiments, an amino lipid is Example 4-84, or a pharmaceutically acceptable salt thereof. In some embodiments, an amino lipid is Example 4-85, or a pharmaceutically acceptable salt thereof. In some embodiments, an amino lipid is Example 4-86, or a pharmaceutically acceptable salt thereof.

### Exemplary Lipids of WO 2021141969

In another aspect, an amino lipid is according to any formula or structure, or a pharmaceutically acceptable salt or solvate thereof, as described in International Publication No. WO2021141969, which is hereby incorporated by reference in its entirety. In some embodiments, an amino lipid has a structure according to any of Formulae I of WO2021141969, or a pharmaceutically acceptable salt or solvate thereof. Exemplary amino lipids also include any of the lipids represented by the Examples of WO2021141969.

In some embodiments, an amino lipid is according to a compound of Formula I of WO2021141969:

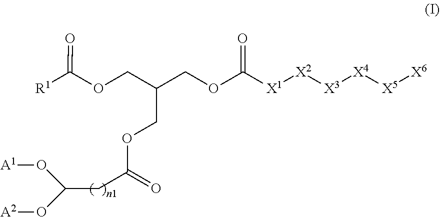

In various embodiments, the compound of Formula (I) is an ionizable lipid as described elsewhere herein. In various embodiments, R¹ in Formula (I) is C₉-C₂₀ alkyl or C₉-C₂₀ alkenyl with 1-3 units of unsaturation. For example, in some embodiments R¹ in Formula (I) is C₉-C₂₀ alkenyl with 2 units of unsaturation, such as a C₁₇ alkenyl group of the formula

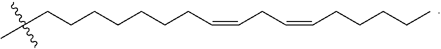 [SMILES: *CCCCCCC/C=C\C/C=C\CCCCC]

In various embodiments, X¹ and X² in Formula (I) are each independently absent or selected from —O—, —NR²—, and

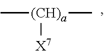

wherein R² is hydrogen or C₁-C₆ alkyl, a is an integer between 1 and 6, X⁷ is independently hydrogen, hydroxyl or —NR⁶R⁷, and R⁶ and R⁷ are each independently hydrogen or C₁-C₆ alkyl; or alternatively R⁶ and R⁷ join together with the nitrogen to which they are bound to form a 4- to 7-membered heterocyclyl optionally substituted with 1 or 2 C₁-C₆ alkyl groups, wherein the heterocyclyl optionally includes an additional heteroatom selected from oxygen, sulfur, and nitrogen. In some embodiments, X¹ is absent, X² is absent, or both X¹ and X² are absent. As described elsewhere herein, X¹-X²-X³-X⁴ does not contain any oxygen-oxygen, oxygen-nitrogen or nitrogen-nitrogen bonds to one another. Accordingly, X¹ and X² cannot both be —O— and cannot both be —NR²—. Similarly, X¹ and X² cannot be —O— and —NR²—, respectively, nor —NR²— and —O—, respectively.

In various embodiments, X¹ is —O—. In various embodiments, X² is —O—. In some embodiments, X¹ is

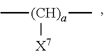

such as —(CH₂)ₐ—, —CH(OH)— or —(CH₂)ₐ₋₁CH(OH)—. In some embodiments, X² is

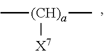

such as —(CH₂)ₐ—, —CH(OH)— or —(CH₂)ₐ₋₁CH(OH)—. In various embodiments, each a is independently 1, 2, 3, 4, 5 or 6. In various embodiments, X¹ is —NR⁶R⁷. In various embodiments, X² is —NR⁶R⁷. In some embodiments, R⁶ is hydrogen or C₁-C₆ alkyl. In some embodiments, R⁷ is hydrogen or C₁-C₆ alkyl. In other embodiments, R⁶ and R⁷ join together with the nitrogen to which they are bound to form a 4- to 7-membered heterocyclyl optionally substituted with 1 or 2 C₁-C₆ alkyl groups. In some embodiments, the 4- to 7-membered heterocyclyl formed by the joining together of R⁶ and R⁷ optionally includes an additional heteroatom selected from oxygen, sulfur, and nitrogen.

In various embodiments, X³ and X⁴ in Formula (I) are each independently absent or selected from:
(1) 4- to 8-membered heterocyclyl optionally substituted with 1 or 2 C₁-C₆ alkyl groups;
(2) 5- to 6-membered heteroaryl optionally substituted with 1 or 2 C₁-C₆ alkyl groups;
(3) 5- to 6-membered aryl optionally substituted with 1 or 2 C₁-C₆ alkyl groups;
(4) 4- to 7-membered cycloalkyl optionally substituted with 1 or 2 C₁-C₆ alkyl groups;
(5) —O—; or
(6) —NR³—, wherein each R³ is independently a hydrogen atom or C₁-C₆ alkyl.

In some embodiments, X³ is absent, X⁴ is absent, or both X³ and X⁴ are absent. As described elsewhere herein, X¹-X²-X³-X⁴ does not contain any oxygen-oxygen, oxygen-nitrogen or nitrogen-nitrogen bonds to one another. Accordingly, X² and X³ cannot both be —O—. When X² is —O— or —NR²— then X³ cannot be —NR³—. Similarly, when X³ is —O— or —NR³— then X² cannot be —NR²—. Likewise, X³ and X⁴ cannot both be —O— and cannot both be —NR³—. Similarly, X³ and X⁴ cannot be —O— and —NR³—, respectively, nor —NR³— and —O—, respectively.

In various embodiments, X³ and X⁴ in Formula (I) are each independently a 4- to 8-membered heterocyclyl optionally substituted with 1 or 2 C₁-C₆ or C₁-C₃ alkyl groups. For example, in various embodiments X³ and X⁴ are each independently azetidinyl, methylazetidinyl, pyrrolidinyl, methylpyrrolidinyl, piperidinyl, methylpiperidinyl, piperazinyl, methylpiperazinyl, dimethylpiperazinyl, morpholinyl, diazepanyl, methyldiazepanyl, octahydro-2H-quinolizinyl, azabicyclo[3.2.1]octyl, methyl-azabicyclo[3.2.1]octyl, diazaspiro[3.5] nonyl or methyldiazaspiro[3.5] nonyl.

In various embodiments, X³ and X⁴ in Formula (I) are each independently a 5- to 6-membered heteroaryl optionally substituted with 1 or 2 C₁-C₆ or C₁-C₃ alkyl groups. For example, in various embodiments X³ and X⁴ are each independently pyrrolyl, methylpyrrolyl, imidazolyl, methylimidazolyl, pyridinyl, or methylpyridinyl.

In various embodiments, X³ and X⁴ in Formula (I) are each independently a 5- to 6-membered aryl optionally substituted with 1 or 2 C₁-C₆ or C₁-C₃ alkyl groups. For example, in various embodiments X³ and X⁴ are each independently phenyl, methylphenyl, naphthyl or methylnaphthyl.

In various embodiments, X³ and X⁴ in Formula (I) are each independently a 4- to 7-membered cycloalkyl optionally substituted with 1 or 2 C₁-C₆ or C₁-C₃ alkyl groups. For example, in various embodiments X³ and X⁴ are each independently cyclopentyl, methylcyclopentyl, cyclohexyl, or methylcyclohexyl.

In various embodiments, X³ in Formula (I) is —O—. In other embodiments, X⁴ in Formula (I) is —O—. In various embodiments, X³ is —NR³—, wherein R³ is a hydrogen atom or C₁-C₆ alkyl, such as a C₁-C₃ alkyl. For example, in various embodiments X³ is —N(CH₃)—, —N(CH₂CH₃)—, or N(CH₂CH₂CH₃)—. In other embodiments, X⁴ is —NR³—, wherein R³ is a hydrogen atom or C₁-C₆ alkyl, such as a C₁-C₃ alkyl. For example, in various embodiments X⁴ is —N(CH₃)—, —N(CH₂CH₃)—, or N(CH₂CH₂CH₃)—.

In various embodiments, X⁵ in Formula (I) is —(CH₂)_{b}—, wherein b is an integer between 0 and 6. In some embodiments, b is 0, in which case X⁵ is absent. In other embodiments, b is 1, 2, 3, 4, 5 or 6.

In various embodiments, X⁶ in Formula (I) is hydrogen, C₁-C₆ alkyl, 5- to 6-membered heteroaryl optionally substituted with 1 or 2 C₁-C₆ alkyl groups, or —NR⁴R⁵. In some embodiments, R⁴ and R⁵ are each independently hydrogen or C₁-C₆ alkyl. Alternatively, in other embodiments R⁴ and R⁵ join together with the nitrogen to which they are bound to form a 4- to 7-membered heterocyclyl optionally substituted with 1 or 2 C₁-C₆ alkyl groups, wherein the 4- to 7-membered heterocyclyl optionally includes an additional heteroatom selected from oxygen, sulfur, and nitrogen.

In various embodiments of Formula (I), at least one of X¹, X², X³, X⁴, and X⁵ is present. For example, in various embodiments at least two of X¹, X², X³, X⁴, and X⁵ are present in Formula (I). In other embodiments, at least three of X¹, X², X³, X⁴, and X⁵ are present in Formula (I). For example, in some embodiments, at least four of X¹, X², X³, X⁴, and X⁵ are present in Formula (I). In other embodiments, all of X¹, X², X³, X⁴, and X⁵ are present in Formula (I).

In some embodiments, X⁶ is hydrogen. In other embodiments, X⁶ is C₁-C₆ alkyl, such as C₁-C₃ alkyl (e.g., methyl, ethyl or propyl). In other embodiments, X⁶ is 5- to 6-membered heteroaryl optionally substituted with 1 or 2 C₁-C₆ alkyl groups. For example, in various embodiments X⁶ is pyrrolyl, methylpyrrolyl, imidazolyl, methylimidazolyl, pyridinyl, or methylpyridinyl. In other embodiments, X⁶ is —NR⁴R⁵. For example, in some embodiments X⁶ is —NH₂, —NHCH₃, —NHCH₂CH₃, —NHCH₂CH₂CH₃, —N(CH₃)₂, —N(CH₂CH₃)₂, or —N(CH₂CH₂CH₃)₂. Alternatively, in other embodiments, R⁴ and R⁵ join together with the nitrogen to which they are bound to form a 4- to 7-membered heterocyclyl. The 4- to 7-membered heterocyclyl can be optionally substituted with 1 or 2 C₁-C₆ alkyl groups, such as C₁-C₃ alkyl, and/or the 4- to 7-membered heterocyclyl can optionally include an additional heteroatom selected from oxygen, sulfur, and nitrogen. For example, in some embodiments X⁶ is azetidinyl, methylazetidinyl, pyrrolidinyl, methylpyrrolidinyl, piperidinyl, methylpiperidinyl, piperazinyl, methylpiperazinyl, dimethylpiperazinyl, morpholinyl, diazepanyl, or methyldiazepanyl.

In various embodiments, each X⁷ in Formula (I) is hydrogen. In other embodiments, each X⁷ is hydroxyl. In other embodiments, each X⁷ is —NR⁶R⁷. For embodiments in which a is between 2 and 6, each X⁷ can be the same or different. For example, in various embodiments X⁷ is —(CH₂)ₐ₋₁CH(X⁷)—, where a is 2, 3, 4, 5 or 6. In some embodiments for which X⁷ is —NR⁶R⁷, R⁶ and R⁷ are each independently hydrogen or C₁-C₆ alkyl, such as C₁-C₃ alkyl. For example, in some embodiments X⁷ is —NH₂, —NHCH₃, —NHCH₂CH₃, —NHCH₂CH₂CH₃, —N(CH₃)₂, —N(CH₂CH₃)₂, or —N(CH₂CH₂CH₃)₂. Alternatively, in some embodiments for which X⁷ is —NR⁶R⁷, R⁶ and R⁷ join together with the nitrogen to which they are bound to form a 4- to 7-membered heterocyclyl optionally substituted with 1 or 2 C₁-C₆ alkyl groups. Alternatively, in other embodiments for which X⁷ is —NR⁶R⁷, the R⁶ and R⁷ can join together with the nitrogen to which they are bound to form a 4- to 7-membered heterocyclyl. The 4- to 7-membered heterocyclyl can be optionally substituted with 1 or 2 C₁-C₆ alkyl groups, such as C₁-C₃ alkyl, and/or the 4- to 7-membered heterocyclyl can optionally include an additional heteroatom selected from oxygen, sulfur, and nitrogen. For example, in some embodiments X⁶ is azetidinyl, methylazetidinyl, pyrrolidinyl, methylpyrrolidinyl, piperidinyl, methylpiperidinyl, piperazinyl, methylpiperazinyl, dimethylpiperazinyl, morpholinyl, diazepanyl, or methyldiazepanyl.

In various embodiments, A¹ and A² in Formula (I) are each independently selected from:
(1) C₅-C₁₂ haloalkyl;
(2) C₅-C₁₂ alkenyl;
(3) C₅-C₁₂ alkynyl;
(4) (C₅-C₁₂ alkoxy)—(CH₂)ₙ₂—;
(5) (C₅-C₁₀ aryl)—(CH₂)ₙ₃-optionally ring substituted with one or two halo, C₁-C₆ alkyl, C₁-C₆ haloalkyl, or C₁-C₆ alkoxy groups; and
(6) (C₃-C₈ cycloalkyl)—(CH₂)ₙ₄-optionally ring substituted with 1 or 2 C₁-C₆ alkyl groups;
or alternatively A¹ and A² join together with the atoms to which they are bound to form a 5- to 6-membered cyclic acetal substituted with 1 or 2 C₄-C₁₀ alkyl groups.

In various embodiments of Formula (I), n1, n2 and n3 are each individually an integer between 1 and 4 (i.e., 1, 2, 3 or 4), and n4 is an integer between zero and 4 (i.e., 0, 1, 2, 3 or 4). In various embodiments, A¹ and A² have the same chemical structure.

In various embodiments of Formula (I), A¹ and A² are each independently a C₅-C₁₂ haloalkyl. For example, in various embodiments the C₅-C₁₂ haloalkyl is a C₅-C₁₂ fluoroalkyl such as a C₆ fluoroalkyl, a C₇ fluoroalkyl, a C₈ fluoroalkyl, a C₉ fluoroalkyl, a C₁₀ fluoroalkyl, a C₁₁ fluoroalkyl, or a C₁₂ fluoroalkyl. The number of halogen atoms attached to the C₅-C₁₂ haloalkyl can vary over a broad range, depending on the length of the alkyl chain and the degree of halogenation. For example, in various embodiments the C₅-C₁₂ haloalkyl contains between 1 and 25 halogen atoms, such as 1, 2, 3, 4, 5, 6, 7, 8, 9, 10 or 11 halogen atoms. In various embodiments, the C₅-C₁₂ haloalkyl is a C₅-C₁₂ fluoroalkyl that comprises a fluorinated end group such as CF₃ (CF₂)ₙ₅—, where n5 is an integer in the range of 0 to 5. For example in various embodiments the C₅-C₁₂ fluoroalkyl is CF₃ (CF₂)ₙ₅ (CH₂)ₙ₆—, where n5 is an integer in the range of 0 to 5, n6 is an integer in the range of 0 to 11, and n5+n6+1 is equal to number of carbons in the C₅-C₁₂ fluoroalkyl.

In various embodiments of Formula (I), A¹ and A² are each independently a C₅-C₁₂ alkenyl. The position of the alkenyl double bond(s) can vary. For example, in various embodiments the C₅-C₁₂ alkenyl is CH₃CH₂CH—CH(CH₂)ₙ₇—, where n7 is an integer in the range of 1 to 8, such as CH₃CH₂CH═CH(CH₂)₄—. In some embodiments, the C₅-C₁₂ alkenyl is branched, such as, for example, (CH₃)₂C═CH(CH₂)ₙ₈—CH(CH₃)—(CH₂)ₙ₉— wherein n8 and n9 are each independently 1, 2 or 3.

In various embodiments of Formula (I), A¹ and A² are each independently a C₅-C₁₂ alkynyl. The position of the alkynyl triple bond(s) can vary. For example, in various embodiments the C₅-C₁₂ alkynyl is CH₃CH₂C═C(CH₂)ₙ₁₀—, where n10 is an integer in the range of 1 to 8, such as CH₃CH₂C═C(CH₂)₄—. In some embodiments, the C₅-C₁₂ alkynyl is branched, such as, for example, (CH₃)₂CHC═C(CH₂)ₙ₁₁—CH(CH₃)—(CH₂)ₙ₁₂— wherein n11 and n12 are each independently 1, 2 or 3 and n11+n12 is in the range of 2 to 5.

In various embodiments of Formula (I), A¹ and A² are each independently a (C₅-C₁₂ alkoxy)-(CH₂)ₙ₂—. In various embodiments, each n2 is independently an integer in the range of 1 to 4 (i.e., 1, 2, 3 or 4). The position of the oxygen(s) can vary. For example, in various embodiments the (C₅-C₁₂ alkoxy)-(CH₂)ₙ₂— is CH₃₀ (CH₂)ₙ₁₃—(CH₂)ₙ₂—, where n13 is an integer in the range of 1 to 11, such as CH₃₀ (CH₂)₇—. In other embodiments the (C₅-C₁₂ alkoxy)-(CH₂)ₙ₂— is CH₃(CH₂)ₙ₁₄—O—(CH₂)ₙ₁₅—(CH₂)ₙ₂—, wherein n14 and n15 are each independently integers between 1 and 8, and n14+n15 is an integer in the range of 4 to 11, such as CH₃(CH₂)₇—O—(CH₂)₂—(CH₂)ₙ₂—. In some embodiments, the C₅-C₁₂ alkoxy is branched, such as, for example, CH₃₀ (CH₂)ₙ₁₆—CH(CH₃)—(CH₂)ₙ₁₇—(CH₂)ₙ₂—, wherein n16 and n17 are each independently 1, 2, 3, 4 or 5 and n16+n17 is an integer in the range of 2 to 9.

In various embodiments of Formula (I), A¹ and A² are each independently a (C₅-C₁₀ aryl)-(CH₂)ₙ₃-optionally ring substituted with one or two halo, C₁-C₆ alkyl, C₁-C₆ haloalkyl, or C₁-C₆ alkoxy groups. In various embodiments, each n3 is independently an integer between 1 and 4 (i.e., 1, 2, 3 or 4). In some embodiments, the C₅-C₁₀ aryl is a phenyl. For example, in various embodiments the (C₅-C₁₀ aryl)-(CH₂)ₙ₃— is C₆H5—(CH₂)ₙ₃— optionally ring substituted with one or two halo, C₁-C₆ alkyl, C₁-C₆ haloalkyl, or C₁-C₆ alkoxy groups. In an embodiment, the optionally ring substituted (C₅-C₁₀ aryl)-(CH₂)ₙ₃— is CF₃-C₆H₄—(CH₂)ₙ₃—, such as CF₃-C₆H4—CH₂— or CF₃—C₆H₄—(CH₂)₂—. In another embodiment, the optionally ring substituted (C₅-C₁₀ aryl)-(CH₂)ₙ₃— is CH₃—(CH₂)ₙ₁₈—C₆H₄—(CH₂)ₙ₂—, wherein n18 is 1, 2 or 3 and n2 is 1, 2, 3 or 4, such as CH₃ (CH₂)₃—C₆H₄—CH₂— or CH₃ (CH₂)₃—C₆H₄—(CH₂)₂—.

In various embodiments of Formula (I), A¹ and A² are each independently a (C₃-C₈ cycloalkyl)-(CH₂)ₙ₄-optionally ring substituted with 1 or 2 C₁-C₆ alkyl groups. In various embodiments, each n4 is independently an integer between 0 and 4 (i.e., 0, 1, 2, 3 or 4). In some embodiments, the C₃-C₈ cycloalkyl is a cyclohexyl or cyclopentyl. For example, in various embodiments the (C₃-C₈ cycloalkyl)-(CH₂)ₙ₄— is C₆H₁₁—(CH₂)ₙ₄-optionally ring substituted with 1 or 2 C₁-C₆ alkyl groups, such as C₆H₁₁—(CH₂)₂—, C₆H₁₁—(CH₂)₃— or CH₃—C₆H₁₀—(CH₂)₃—.

Alternatively, in other embodiments of Formula (I), A¹ and A² join together with the atoms to which they are bound to form a 5- to 6-membered cyclic acetal substituted with 1 or 2 C₄-C₁₀ alkyl groups. For example, in an embodiment, A¹ and A² join together with the atoms to which they are bound to form a 6-membered cyclic acetal that is ring substituted with 2 C₈ alkyl groups as follows:

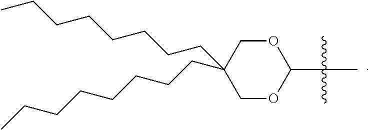 [SMILES: *C1OCC(CCCCCCCC)(CCCCCCCC)CO1]

In another embodiment,

A¹ and A² join together with the atoms to which they are bound to form a 5-membered cyclic acetal that is ring substituted with 2 C₈ alkyl groups as follows:

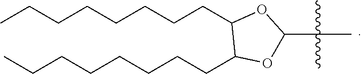 [SMILES: *C1OC(CCCCCCCC)C(CCCCCCCC)O1]

### Exemplary Lipids of WO2021113365

In another aspect, an amino lipid is according to any formula or structure, or a pharmaceutically acceptable salt or solvate thereof, as described in International Publication No. WO2021113365, which is hereby incorporated by reference in its entirety. In some embodiments, an amino lipid has a structure according to any of Formulae I of WO2021113365, or a pharmaceutically acceptable salt or solvate thereof. Exemplary amino lipids also include any of the lipids represented by the Examples of WO2021113365.

In some embodiments, an amino lipid is according to a compound of Formula I of WO2021113365:

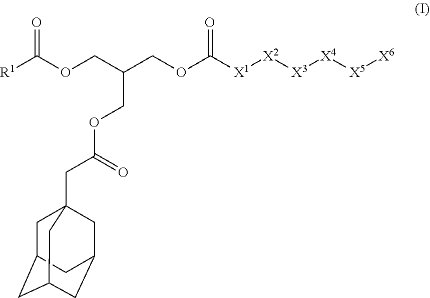

wherein:
R¹ is C₉-C₂₀ alkyl or C₉-C₂₀ alkenyl with 1-3 units of unsaturation;
X¹ and X² are each independently absent or selected from —O—, NR², and

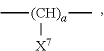

wherein R² is C₁-C₆ alkyl, and wherein X¹ and X² are not both —O— or NR²;
a is an integer between 1 and 6;
X³ and X⁴ are each independently absent or selected from the group consisting of: 4- to 7-membered heterocyclyl optionally substituted with 1 or 2 C₁-C₆ alkyl groups, 5- to 6-membered heteroaryl optionally substituted with 1 or 2 C₁-C₆ alkyl groups, and —NR³—, wherein each R³ is a hydrogen atom or C₁-C₆ alkyl;
X⁵ is —(CH₂)_{b}—, wherein b is an integer between 0 and 6;
X⁶ is hydrogen, C₁-C₆ alkyl, 5- to 6-membered heteroaryl optionally substituted with 1 or 2 C₁-C₆ alkyl groups, or —NR⁴R⁵, wherein R⁴ and R⁵ are each independently hydrogen or C₁-C₆ alkyl; or alternatively R⁴ and R⁵ join together with the nitrogen to which they are bound to form a 4- to 7-membered heterocyclyl optionally substituted with 1 or 2 C₁-C₆ alkyl groups, wherein the heterocyclyl optionally includes an additional heteroatom selected from oxygen, sulfur, and nitrogen;
X⁷ is hydrogen or —NR⁶R⁷, wherein R⁶ and R⁷ are each independently hydrogen or C₁-C₆ alkyl; or alternatively R⁶ and R⁷ join together with the nitrogen to which they are bound to form a 4- to 7-membered heterocyclyl optionally substituted with 1 or 2 C₁-C₆ alkyl groups, wherein the heterocyclyl optionally includes an additional heteroatom selected from oxygen, sulfur, and nitrogen;
at least one of X¹, X², X³, X⁴, and X⁵ is present; and
provided that when either X¹ or X² is —O—, neither X³ nor X⁴ is

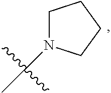 [SMILES: *N1CCCC1]

and when either X¹ or X² is —O—, R⁴ and R⁵ are not both ethyl.

### Exemplary Lipids of WO2022140239

In another aspect, an amino lipid is according to any formula or structure, or a pharmaceutically acceptable salt or solvate thereof, as described in International Publication No. WO2022140239, which is hereby incorporated by reference in its entirety. In some embodiments, an amino lipid has a structure according to any of Formulae I′ of WO2022140239, or a pharmaceutically acceptable salt or solvate thereof. Exemplary amino lipids also include any of the lipids represented by the Examples of WO2022140239.

In some embodiments, an amino lipid is according to a compound of Formula I′ of WO2022140239:

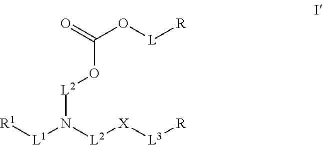

or its N-oxide, or a pharmaceutically acceptable salt thereof, wherein
L¹ is absent, C₁-6 alkylenyl, or C₂-6 heteroalkylenyl;
each L² is independently optionally substituted C₂₋₁₅ alkylenyl, or optionally substituted C₃₋₁₅ heteroalkylenyl;
L is absent, optionally substituted C₁₋₁₀ alkylenyl, or optionally substituted C₂₋₁₀ heteroalkylenyl;
L³ is absent, optionally substituted C₁₋₁₀ alkylenyl, or optionally substituted C₂₋₁₀ heteroalkylenyl;
X is absent, —OC(O)—, —C(O)O—, or —OC(O)O—;
each R is independently hydrogen,

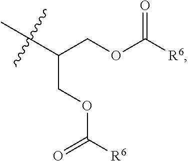

or an optionally substituted group selected from C₆-20 aliphatic, 3- to 12-membered cycloaliphatic, 7- to 12-membered bridged bicyclic comprising 0-4 heteroatoms independently selected from nitrogen, oxygen, or sulfur, 1-adamantyl, 2-adamantyl, sterolyl, and phenyl;

R¹ is hydrogen, optionally substituted phenyl, optionally substituted 3- to 7-membered cycloaliphatic, optionally substituted 3- to 7-membered heterocyclyl comprising 1-3 heteroatoms independently selected from nitrogen, oxygen, and sulfur, optionally substituted 5- to 6-membered monocyclic heteroaryl comprising 1-4 heteroatoms independently selected from nitrogen, oxygen, and sulfur, optionally substituted 8- to 10-membered bicyclic heteroaryl comprising 1-4 heteroatoms independently selected from nitrogen, oxygen, and sulfur, —OR², —C(O)OR², —C(O) SR², —OC(O)R², —OC(O)OR², —CN, —N(R²)₂, —C(O)N(R²)₂, —S(O)₂N(R²)₂, —NR²C(O)R², —OC(O)N(R²)₂, —N(R²)C(O)OR², —NR²S(O)₂R², —NR²C(O)N(R²)₂, —NR²C(S)N(R²)₂, —NR²C(NR²)N(R²)₂, —NR²C(CHR²)N(R²)₂, —N(OR²)C(O)R², —N(OR²) S(O)₂R², —N(OR²)C(O)OR², —N(OR²)C(O)N(R²)₂, —N(OR²)C(S)N(R²)₂, —N(OR²)C(NR²)N(R²)₂, —N(OR²)C(CHR²)N(R²)₂, —C(NR²)N(R²)₂, —C(NR²)R², —C(O)N(R²)OR², —C(R²)N(R²)₂C(O)OR², —CR² (R³)₂, —OP(O)(OR²)₂, or —P(O)(OR²)₂; or
R¹ is

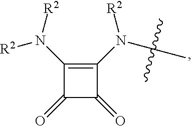

or a ring selected from 3- to 7-membered cycloaliphatic and 3- to 7-membered heterocyclyl comprising 1-3 heteroatoms independently selected from nitrogen, oxygen, and sulfur, wherein the cycloaliphatic or heterocyclyl ring is optionally substituted with 1-4 R² or R³ groups;
each R² is independently hydrogen,
   oxo, —CN, —NO₂, —OR⁴, —S(O)₂R⁴, —S(O)₂N(R⁴)₂, —(CH₂) n-R⁴, or an optionally substituted group selected from C₁-6 aliphatic, phenyl, 3- to 7-membered cycloaliphatic, 5- to 6-membered monocyclic heteroaryl comprising 1-4 heteroatoms independently selected from nitrogen, oxygen, and sulfur, and 3- to 7-membered heterocyclyl comprising 1-3 heteroatoms independently selected from nitrogen, oxygen, and sulfur; or
   two occurrences of R², taken together with the atom(s) to which they are attached, form optionally substituted 4- to 7-membered heterocyclyl comprising 0-1 additional heteroatom selected from nitrogen, oxygen, and sulfur;
each R³ is independently —(CH₂)ₙ—R⁴; or
   two occurrences of R³, taken together with the atom(s) to which they are attached, form optionally substituted 5- to 6-membered heterocyclyl comprising 0-1 additional heteroatom selected from nitrogen, oxygen, and sulfur;
each R⁴ is independently
hydrogen, —OR⁵, —N(R⁵)₂, —OC(O)R⁵, —OC(O)OR⁵, —CN, —C(O)N(R⁵)₂, —NR⁵C(O)R⁵, —OC(O)N(R⁵)₂, —N(R⁵)C(O)OR⁵, —NR⁵S(O)₂R⁵, —NR⁵° C. (O)N(R⁵)₂, —NR⁵C(S)N(R⁵)₂, —NR⁵C(NR⁵)N(R⁵)₂, or

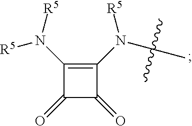

each R⁵ is independently hydrogen, or optionally substituted C₁₋₆ aliphatic; or two occurrences of R⁵, taken together with the atom(s) to which they are attached, form optionally substituted 4- to 7-membered heterocyclyl comprising 0-1 additional heteroatom selected from nitrogen, oxygen, and sulfur;
each R⁶ is independently C₄-12 aliphatic; and
each n is independently 0 to 4.

### Exemplary Lipids of WO2022140238

In another aspect, an amino lipid is according to any formula or structure, or a pharmaceutically acceptable salt or solvate thereof, as described in International Publication No. WO2022140238, which is hereby incorporated by reference in its entirety. In some embodiments, an amino lipid has a structure according to any of Formulae I′ of WO2022140238, or a pharmaceutically acceptable salt or solvate thereof. Exemplary amino lipids also include any of the lipids represented by the Examples of WO2022140238.

In some embodiments, an amino lipid is according to a compound of Formula I′ of WO2022140238:

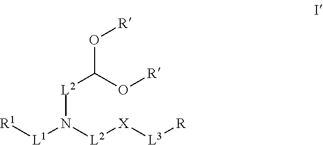

or its N-oxide, or a pharmaceutically acceptable salt thereof, wherein
L¹ is absent, C₁-6 alkylenyl, or C₂-6 heteroalkylenyl;
each L² is independently optionally substituted C₂₋₁₅ alkylenyl, or optionally substituted C₃₋₁₅ heteroalkylenyl;
L³ is absent, optionally substituted C₁₋₁₀ alkylenyl, or optionally substituted C₂₋₁₀ heteroalkylenyl;
X is absent, —OC(O)—, —C(O)O—, or —OC(O)O—;
each R′ is independently an optionally substituted group selected from C₄-12 aliphatic, 3- to 12-membered cycloaliphatic, 7- to 12-membered bridged bicyclic comprising 0-4 heteroatoms independently selected from nitrogen, oxygen, or sulfur, 1-adamantyl, 2-adamantyl, sterolyl, and phenyl;
R is hydrogen,

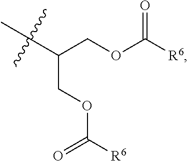

or an optionally substituted group selected from C₆-20 aliphatic, 3- to 12-membered cycloaliphatic, 7- to 12-membered bridged bicyclic comprising 0-4 heteroatoms independently selected from nitrogen, oxygen, or sulfur, 1-adamantyl, 2-adamantyl, sterolyl, and phenyl;
R¹ is hydrogen, optionally substituted phenyl, optionally substituted 3- to 7-membered cycloaliphatic, optionally substituted 3- to 7-membered heterocyclyl comprising 1-3 heteroatoms independently selected from nitrogen, oxygen, and sulfur, optionally substituted 5- to 6-membered monocyclic heteroaryl comprising 1-4 heteroatoms independently selected from nitrogen, oxygen, and sulfur, optionally substituted 8- to 10-membered bicyclic heteroaryl comprising 1-4 heteroatoms independently selected from nitrogen, oxygen, and sulfur, —OR², —C(O)OR², —C(O)SR², —OC(O)R², —OC(O)OR², —CN, —N(R²)₂, —C(O)N(R²)₂, —S(O)₂N(R²)₂, —NR²C(O)R², —OC(O)N(R²)₂, —N(R²)C(O)OR², —NR²S(O)₂R², —NR²C(O)N(R²)₂, —NR²C(S)N(R²)₂, —NR²C(NR²)N(R²)₂, —NR²C(CHR²)N(R²)₂, —N(OR²)C(O)R², —N(OR²) S(O)₂R², —N(OR²)C(O)OR², —N(OR²)C(O)N(R²)₂,
   —N(OR²)C(S)N(R²)₂, —N(OR²)C(NR²)N(R²)₂, —N(OR²)C(CHR²)N(R²)₂, —C(NR²)N(R²)₂, —C(NR²)R², —C(O)N(R²)OR², —C(R²)N(R²)₂C(O)OR², —CR² (R³)₂, —OP(O) (OR²)₂, or —P(O)(OR²)₂; or
R¹ is

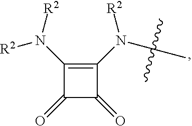

or a ring selected from 3- to 7-membered cycloaliphatic and 3- to 7-membered heterocyclyl comprising 1-3 heteroatoms independently selected from nitrogen, oxygen, and sulfur, wherein the cycloaliphatic or heterocyclyl ring is optionally substituted with 1-4 R² or R³ groups;
each R² is independently hydrogen, oxo, —CN, —NO₂, —OR⁴, —S(O)₂R⁴, —S(O)₂N(R⁴)₂, —(CH₂) n-R⁴, or an optionally substituted group selected from C₁-6 aliphatic, phenyl, 3- to 7-membered cycloaliphatic, 5- to 6-membered monocyclic heteroaryl comprising 1-4 heteroatoms independently selected from nitrogen, oxygen, and sulfur, and 3- to 7-membered heterocyclyl comprising 1-3 heteroatoms independently selected from nitrogen, oxygen, and sulfur; or
   two occurrences of R², taken together with the atom(s) to which they are attached, form optionally substituted 4- to 7-membered heterocyclyl comprising 0-1 additional heteroatom selected from nitrogen, oxygen, and sulfur;
each R³ is independently-(CH₂) n-R⁴; or
   two occurrences of R³, taken together with the atom(s) to which they are attached, form optionally substituted 5- to 6-membered heterocyclyl comprising 0-1 additional heteroatom selected from nitrogen, oxygen, and sulfur;
each R⁴ is independently hydrogen, —OR⁵, —N(R⁵)₂, —OC(O)R⁵, —OC(O)OR⁵, —CN, —C(O)N(R⁵)₂,
   —NR⁵C(O)R⁵, —OC(O)N(R⁵)₂, —N(R⁵)C(O)OR⁵, —NR⁵S(O)₂R⁵, —NR⁵C(O)N(R⁵)₂,
   —NR⁵C(S)N(R⁵)₂, —NR⁵C(NR⁵)N(R⁵)₂, or

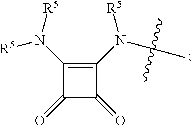

each R⁵ is independently hydrogen, or optionally substituted C₁₋₆ aliphatic; or
   two occurrences of R⁵, taken together with the atom(s) to which they are attached, form optionally substituted 4- to 7-membered heterocyclyl comprising 0-1 additional heteroatom selected from nitrogen, oxygen, and sulfur;
each R⁶ is independently C₄-12 aliphatic; and
each n is independently 0 to 4.

### Exemplary Lipids of WO2022159421

In another aspect, an amino lipid is according to any formula or structure, or a pharmaceutically acceptable salt or solvate thereof, as described in International Publication No. WO2022159421, which is hereby incorporated by reference in its entirety. In some embodiments, an amino lipid has a structure according to any of Formulae I of WO2022159421, or a pharmaceutically acceptable salt or solvate thereof. Exemplary amino lipids also include any of the lipids represented by the Examples of WO2022159421.

In some embodiments, an amino lipid is according to a compound of Formula I of WO2022159421:

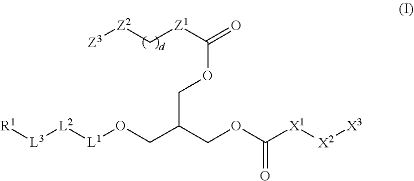

or a pharmaceutically acceptable salt thereof, wherein:
L¹ is a covalent bond, —C(O)—, or —OC(O)—;
L² is a covalent bond, an optionally substituted bivalent saturated or unsaturated, straight or branched C₁-C₁₂ hydrocarbon chain, or

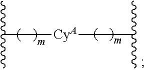

Cy^{A} is an optionally substituted ring selected from phenylene and 3- to 7-membered saturated or partially unsaturated carbocyclene;
each m is independently 0, 1, or 2;
L³ is a covalent bond, —C(O)—, —C(O)O—, —OC(O)—, —O—, or —OC(O)O—;
R¹ is

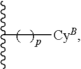

or an optionally substituted saturated or unsaturated, straight or branched C₁-C₂₀ hydrocarbon chain wherein 1-3 methylene units are optionally and independently replaced with —O— or —NR—;
Cy^{B} is an optionally substituted ring selected from 3- to 12-membered saturated or partially unsaturated carbocyclyl, 1-adamantyl, 2-adamantyl,

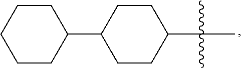 [SMILES: *C1CCC(C2CCCCC2)CC1]

sterolyl, and phenyl;
p is 0, 1, 2, or 3;
X¹ is a covalent bond, —O—, or —NR—;
X² is a covalent bond or an optionally substituted, bivalent saturated or unsaturated, straight or branched, C₁-C₁₂ hydrocarbon chain, wherein 1-3 methylene units are optionally and independently replaced with —O—, —NR—, or —Cy^{C}—;
Cy^{C} is an optionally substituted ring selected from 3- to 7-membered saturated or partially unsaturated carbocyclene, phenylene, 3- to 7-membered saturated or partially unsaturated heterocyclene having 1-3 heteroatoms independently selected from nitrogen, oxygen, and sulfur, and 5- to 6-membered heteroarylene having 1-3 heteroatoms independently selected from nitrogen, oxygen, and sulfur;
X³ is hydrogen or an optionally substituted ring selected from 3- to 7-membered saturated or partially unsaturated carbocyclyl, phenyl, 3- to 7-membered saturated or partially unsaturated heterocyclyl having 1-3 heteroatoms independently selected from nitrogen, oxygen, and sulfur, or 5- to 6-membered heteroaryl having 1-3 heteroatoms independently selected from nitrogen, oxygen, and sulfur;
each R is independently hydrogen or an optionally substituted C₁-C₆ aliphatic group;
Z¹ is a covalent bond or —O—;
Z² is an optionally substituted group selected from 4- to 12-membered saturated or partially unsaturated carbocyclyl, phenyl, 1-adamantyl, and 2-adamantyl;
Z³ is hydrogen, or an optionally substituted group selected from C₁-C₁₀ aliphatic, and 4- to 12-membered saturated or partially unsaturated carbocyclyl; and
d is 0, 1, 2, 3, 4, 5, or 6;
provided that when L³ is a covalent bond, then R¹ must be

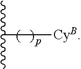

### Exemplary Lipids of WO2022159475

In another aspect, an amino lipid is according to any formula or structure, or a pharmaceutically acceptable salt or solvate thereof, as described in International Publication No. WO2022159475, which is hereby incorporated by reference in its entirety. In some embodiments, an amino lipid has a structure according to any of Formulae I of WO2022159475, or a pharmaceutically acceptable salt or solvate thereof. Exemplary amino lipids also include any of the lipids represented by the Examples of WO2022159475.

In some embodiments, an amino lipid is according to a compound of Formula I of WO2022159475:

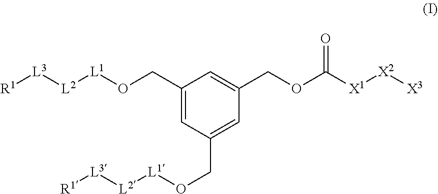

or a pharmaceutically acceptable salt thereof, wherein:
each L¹ and L^{1′} is independently —C(O)— or —C(O)O—;
each L² and L^{2′} is independently a covalent bond, an optionally substituted bivalent saturated or unsaturated, straight or branched C₁-C₁₂ hydrocarbon chain, or

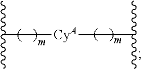

each Cy^{A} is independently an optionally substituted ring selected from phenylene and a 3- to 7-membered saturated or partially unsaturated carbocyclene;
each m is independently 0, 1, or 2;
each L³ and L^{3′} is independently a covalent bond, —C(O)O—, —OC(O)—, —O—, or —OC(O)O—;
each R¹ and R^{1′} is independently an optionally substituted group selected from a saturated or unsaturated, straight or branched C₁-C₂₀ hydrocarbon chain, wherein 1-3 methylene units are optionally and independently replaced with —O— or —NR—, a 3- to 12-membered saturated or partially unsaturated carbocyclic ring, 1-adamantyl, 2-adamantyl, sterolyl, phenyl, and

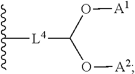

each L⁴ is independently a bivalent saturated or unsaturated, straight or branched C₁-C₆ hydrocarbon chain;
each A¹ and A² is independently an optionally substituted C₁-C₂₀ aliphatic or —L⁵-R⁵; or A¹ and A², together with their intervening atoms, may form an optionally substituted ring:

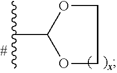

wherein
x is selected from 1 or 2; and
#represents the point of attachment to L⁴;
each L⁵ is independently a bivalent saturated or unsaturated, straight or branched C₁-C₂₀ hydrocarbon chain, wherein 1-3 methylene units are optionally and independently replaced with —O— or —NR—;
each R⁵ is independently an optionally substituted group selected from a 5- to 10-membered aryl ring and a 3- to 8-membered carbocyclic ring;
X¹ is a covalent bond, —O—, or —NR—;
X² is a covalent bond or an optionally substituted, bivalent saturated or unsaturated, straight or branched, C₁-C₁₂ hydrocarbon chain, wherein 1-3 methylene units are optionally and independently replaced with —O— or —NR—;
X³ is hydrogen or —Cy^{B};
Cy^{B} is an optionally substituted ring selected from 3- to 7-membered saturated or partially unsaturated carbocyclyl, phenyl, 3- to 7-membered saturated or partially unsaturated heterocyclyl having 1-3 heteroatoms independently selected from nitrogen, oxygen, and sulfur, and 5- to 6-membered heteroaryl having 1-3 heteroatoms independently selected from nitrogen, oxygen, and sulfur; and
each R is independently hydrogen or an optionally substituted C₁-C₆ aliphatic group;
provided that when X³ is hydrogen, at least one of R¹ or R^{1′} is

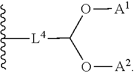

In some embodiments, the present disclosure provides a compound of Formula I or a pharmaceutically acceptable salt thereof, wherein:
each L¹ and L^{1′} is independently —C(O)— or —C(O)O—;
each L² and L^{2′} is independently a covalent bond, an optionally substituted bivalent saturated or unsaturated, straight or branched C₁-C₁₂ hydrocarbon chain, or

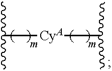

each Cy^{A} is independently an optionally substituted ring selected from phenylene and a 3- to 7-membered saturated or partially unsaturated carbocyclene;
each m is independently 0, 1, or 2;
each L³ and L^{3′} is independently a covalent bond, —C(O)O—, —OC(O)—, —O—, or —OC(O)O—;
each R¹ and R^{1′} is independently an optionally substituted group selected from a saturated or unsaturated, straight or branched C₁-C₂₀ hydrocarbon chain, wherein 1-3 methylene units are optionally and independently replaced with —O— or —NR—, a 3- to 7-membered saturated or partially unsaturated carbocyclic ring, 1-adamantyl, 2-adamantyl, sterolyl, phenyl, and

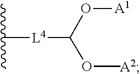

each L⁴ is independently a bivalent saturated or unsaturated, straight or branched C₁-C₆ hydrocarbon chain;
each A¹ and A² is independently an optionally substituted C₁-C₂₀ aliphatic or —L⁵-R⁵;
or A¹ and A², together with their intervening atoms, may form an optionally substituted ring:

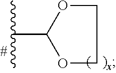

wherein
x is selected from 1 or 2; and
#represents the point of attachment to L⁴;
each L⁵ is independently a bivalent saturated or unsaturated, straight or branched C₁-C₂₀ hydrocarbon chain, wherein 1-3 methylene units are optionally and independently replaced with —O— or —NR—;
each R⁵ is independently an optionally substituted group selected from a 5- to 10-membered aryl ring and a 3- to 8-membered carbocyclic ring;
X¹ is a covalent bond, —O—, or —NR—;
X² is a covalent bond or an optionally substituted, bivalent saturated or unsaturated, straight or branched, C₁-C₁₂ hydrocarbon chain, wherein 1-3 methylene units are optionally and independently replaced with —O— or —NR—;
X³ is hydrogen or —Cy^{B};
Cy^{B} is an optionally substituted ring selected from 3- to 7-membered saturated or partially unsaturated carbocyclyl, phenyl, 3- to 7-membered saturated or partially unsaturated heterocyclyl having 1-3 heteroatoms independently selected from nitrogen, oxygen, and sulfur, and 5- to 6-membered heteroaryl having 1-3 heteroatoms independently selected from nitrogen, oxygen, and sulfur; and
each R is independently hydrogen or an optionally substituted C₁-C₆ aliphatic group; provided that when X³ is hydrogen, at least one of R¹ or R^{1′} is

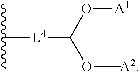

### Exemplary Lipids of WO2022159463

In another aspect, an amino lipid is according to any formula or structure, or a pharmaceutically acceptable salt or solvate thereof, as described in International Publication No. WO2022159463, which is hereby incorporated by reference in its entirety. In some embodiments, an amino lipid has a structure according to any of Formulae I of WO2022159463, or a pharmaceutically acceptable salt or solvate thereof. Exemplary amino lipids also include any of the lipids represented by the Examples of WO2022159463.

In some embodiments, an amino lipid is according to a compound of Formula I of WO2022159463:

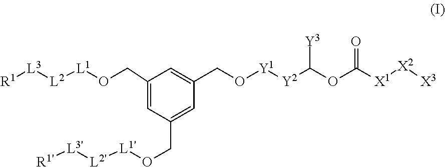

or a pharmaceutically acceptable salt thereof, wherein:
each of L¹ and L^{1′} is independently a covalent bond, —C(O)—, or —OC(O)—;
each of L² and L^{2′} is independently a covalent bond, an optionally substituted bivalent saturated or unsaturated, straight or branched C₁-C₁₂ hydrocarbon chain, or

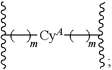

each Cy^{A} is independently an optionally substituted ring selected from phenylene or 3- to 7-membered saturated or partially unsaturated carbocyclene;
each m is independently 0, 1, or 2;
each of L³ and L^{3′} is independently a covalent bond, —O—, —C(O)O—, —OC(O)—, or —OC(O)O—;
each of R¹ and R^{1′} is independently an optionally substituted group selected from saturated or unsaturated, straight or branched C₁-C₂₀ hydrocarbon chain wherein 1-3 methylene units are optionally and independently replaced with —O— or —NR—, a 3- to 7-membered saturated or partially unsaturated carbocyclic ring, 1-adamantyl, 2-adamantyl, sterolyl, phenyl, or

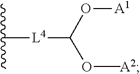

each L⁴ is independently a bivalent saturated or unsaturated, straight or branched C₁-C₂₀ hydrocarbon chain;
each A¹ and A² is independently an optionally substituted C₁-C₂₀ aliphatic or —L⁵-R⁵,
or A¹ and A², together with their intervening atoms, may form an optionally substituted ring:

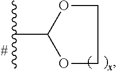

wherein
   x is selected from 1 or 2; and
   #represents the point of attachment to L⁴;
each L⁵ is independently a bivalent saturated or unsaturated, straight or branched C₁-C₂₀ hydrocarbon chain, wherein 1-3 methylene units are optionally and independently replaced with —O— or —NR—;
each R⁵ is independently an optionally substituted group selected from a 6- to 10-membered aryl ring or a 3- to 8-membered carbocyclic ring;
Y¹ is a covalent bond, —C(O)—, or —C(O)O—;
Y² is a bivalent saturated or unsaturated, straight or branched C₁-C₆ hydrocarbon chain, wherein 1-2 methylene units are optionally and independently replaced with cyclopropylene, —O—, or —NR—;
Y³ is an optionally substituted group selected from saturated or unsaturated, straight or branched C₁-C₁₄ hydrocarbon chain, wherein 1-3 methylene units are optionally and independently replaced with —O— or —NR—, a 3- to 7-membered saturated or partially unsaturated carbocyclic ring, 1-adamantyl, 2-adamantyl, or phenyl;
X¹ is a covalent bond, —O—, or —NR—;
X² is an optionally substituted bivalent saturated or unsaturated, straight or branched C₁-C₁₂ hydrocarbon chain, wherein 1-3 methylene units are optionally and independently replaced with —O—, —NR—, or —Cy^{B}—;
each Cy^{B} is independently an optionally substituted ring selected from 3- to 7-membered saturated or partially unsaturated carbocyclene, phenylene, 3- to 7-membered heterocyclene having 1-3 heteroatoms independently selected from nitrogen, oxygen, and sulfur, or 5- to 6-membered heteroarylene having 1-3 heteroatoms independently selected from nitrogen, oxygen, and sulfur;
X³ is hydrogen or an optionally substituted ring selected from 3- to 7-membered saturated or partially unsaturated carbocyclyl, phenyl, 3- to 7-membered heterocyclyl having 1-3 heteroatoms independently selected from nitrogen, oxygen, and sulfur, or 5- to 6-membered heteroaryl having 1-3 heteroatoms independently selected from nitrogen, oxygen, and sulfur; and
each R is independently hydrogen or an optionally substituted C₁-C₆ aliphatic group.

### LNP Compositions Comprising Different Amino Lipids

In some embodiments, the LNP comprises a plurality of amino lipids having different formulas. For example, the LNP composition can comprise 2, 3, 4, 5, 6.7, 8, 9. 10, or more amino lipids. For another example, the LNP composition can comprise at least 2, at least 3, at least 4, at least 5, at least 6, at least 7, at least 9, at least 10, or at least 20 amino lipids. For yet another example, the LNP composition can comprise at most 2, at most 3, at most 4, at most 5, at most 6, at most 7, at most 9, at most 10, at most 20, or at most 30 amino lipids.

In some embodiments, the LNP composition comprises a first amino lipid. In some embodiments, the LNP composition comprises a first amino lipid and a second amino lipid. In some embodiments, the LNP composition comprises a first amino lipid, a second amino lipid, and a third amino lipid. In some embodiments, the LNP composition comprises a first amino lipid, a second amino lipid, a third amino lipid, and a fourth amino lipid. In some embodiments, the LNP composition does not comprise a fourth amino lipid. In some embodiments, the LNP composition does not comprise a third amino lipid. In some embodiments, a molar ratio of the first amino lipid to the second amino lipid is from about 0.1 to about 10. In some embodiments, a molar ratio of the first amino lipid to the second amino lipid is from about 0.20 to about 5. In some embodiments, a molar ratio of the first amino lipid to the second amino lipid is from about 0.25 to about 4. In some embodiments, a molar ratio of the first amino lipid to the second amino lipid is about 0.25, about 0.33, about 0.5, about 1, about 2, about 3, or about 4.

In some embodiments, a molar ratio of the first amino lipid: the second amino lipid: the third amino lipid is about 4:1:1. In some embodiments, a molar ratio of the first amino lipid: the second amino lipid: the third amino lipid is about 1:1:1. In some embodiments, a molar ratio of the first amino lipid: the second amino lipid: the third amino lipid is about 2:1:1. In some embodiments, a molar ratio of the first amino lipid: the second amino lipid: the third amino lipid is about 2:2:1. In some embodiments, a molar ratio of the first amino lipid: the second amino lipid: the third amino lipid is about 3:2:1. In some embodiments, a molar ratio of the first amino lipid: the second amino lipid: the third amino lipid is about 3:1:1. In some embodiments, a molar ratio of the first amino lipid: the second amino lipid: the third amino lipid is about 5:1:1. In some embodiments, a molar ratio of the first amino lipid: the second amino lipid: the third amino lipid is about 3:3:1. In some embodiments, a molar ratio of the first amino lipid: the second amino lipid: the third amino lipid is about 4:4:1.

### Additional Amino Lipid Embodiments

In some embodiments, the LNP composition comprises one or more amino lipids. In some embodiments, the one or more amino lipids comprise from about 40 mol % to about 65 mol % of the total lipid present in the particle. In some embodiments, the one or more amino lipids comprise about 40 mol %, about 41 mol %, about 42 mol %, about 43 mol %, about 44 mol %, about 45 mol %, about 46 mol %, about 47 mol %, about 48 mol %, about 49 mol %, about 50 mol %, about 51 mol %, about 52 mol %, about 53 mol %, about 54 mol %, about 55 mol %, about 56 mol %, about 57 mol %, about 58 mol %, about 59 mol %, about 60 mol %, about 61 mol %, about 62 mol %, about 63 mol %, about 64 mol %, or about 65 mol % of the total lipid present in the particle. In some embodiments, the first amino lipid comprises from about 1 mol % to about 99 mol % of the total amino lipids present in the particle. In some embodiments, the first amino lipid comprises from about 16.7 mol % to about 66.7 mol % of the total amino lipids present in the particle. In some embodiments, the first amino lipid comprises from about 20 mol % to about 60 mol % of the total amino lipids present in the particle.

In some embodiments, the amino lipid is an ionizable lipid. An ionizable lipid can comprise one or more ionizable nitrogen atoms. In some embodiments, at least one of the one or more ionizable nitrogen atoms is positively charged. In some embodiments, at least 10 mol %, 20 mol %, 30 mol %, 40 mol %, 50 mol %, 60 mol %, 70 mol %, 80 mol %. 90 mol %, 95 mol %, or 99 mol % of the ionizable nitrogen atoms in the LNP composition are positively charged. In some embodiments, the amino lipid comprises a primary amine, a secondary amine, a tertiary amine, an imine, an amide, a guanidine moiety, a histidine residue, a lysine residue, an arginine residue, or any combination thereof. In some embodiments, the amino lipid comprises a primary amine, a secondary amine, a tertiary amine, a guanidine moiety, or any combination thereof. In some embodiments, the amino lipid comprises a tertiary amine.

In some embodiments, the amino lipid (e.g. an ionizable lipid) is a cationic lipid. In some embodiments, the cationic lipid is an ionizable lipid. In some embodiments, the amino lipid comprises one or more nitrogen atoms. In some embodiments, the amino lipid comprises one or more ionizable nitrogen atoms. Exemplary cationic and/or ionizable lipids include, but are not limited to, 3-(didodecylamino)-N1,N1,4-tri dodecyl-1-piperazineethan amine (KL10), N142-(didodecylamino) ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoley 1-4-dimethylaminomethyl-[1,3]-dioxolane (DLin-K-DMA), heptatriaconta-6,9,28,31-tetraen-19-y1 4-(dimethylamino) butanoate (DLin-MC 3-DMA), 2,2-dilinoley 1-4-(2-dimethylaminoethyl)-[1,3]-dioxolane (DLin-KC2-DMA), 1,2-dioleyloxy-N,N-dimethylaminopropane (DODMA), 2-({8-[(3B)-cholest-5-en-3-yloxy]octyl}oxy)-N,N-dimethy 1-3-[(9Z,12Z)-octadeca-9,12-dien-1-yloxy]propan-1-amine (Octyl-CLinDMA), (2R)-2-({8-[(3B)-cholest-5-en-3-yloxy]octyl}oxy)-N,N-dimethy 1-3-[(9Z,12Z)-octadeca-9,12-dien-1-yloxy]propan-1-amine (Octyl-CLinDMA (2R)), and (2S)-2-({8-[(3B)-cholest-5-en-3-yloxy]octyl}oxy)—N,N-dimethy 1-3-[(9Z,12Z)-octadeca-9,12-dien-1-yloxy]propan-1-amine (Octyl-CLinDMA (2S)).

In some embodiments, an amino lipid described herein can take the form of a salt, such as a pharmaceutically acceptable salt. All pharmaceutically acceptable salts of the amino lipid are encompassed by this disclosure. As used herein, the term “amino lipid” also includes its pharmaceutically acceptable salts, and its diastereomeric, enantiomeric, and epimeric forms.

In some embodiments, an amino lipid described herein, possesses one or more stereocenters and each stereocenter exists independently in either the R or S configuration. The lipids presented herein include all diastereomeric, enantiomeric, and epimeric forms as well as the appropriate mixtures thereof. The lipids provided herein include all cis. trans, syn, anti, entgegen (E), and zusammen (Z) isomers as well as the appropriate mixtures thereof. In certain embodiments, lipids described herein are prepared as their individual stereoisomers by reacting a racemic mixture of the compound with an optically active resolving agent to form a pair of diastereoisomeric compounds/salts, separating the diastereomers and recovering the optically pure enantiomers. In some embodiments, resolution of enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley and Sons, Inc., 1981. In one aspect, stereoisomers are obtained by stereoselective synthesis.

In some embodiments, the lipids such as the amino lipids are substituted based on the structures disclosed herein. In some embodiments, the lipids such as the amino lipids are unsubstituted. In another embodiment, the lipids described herein are labeled isotopically (e.g., with a radioisotope) or by another other means, including, but not limited to, the use of chromophores or fluorescent moieties, bioluminescent labels, or chemiluminescent labels.

Lipids described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present lipids include isotopes of hydrogen, carbon, nitrogen, oxygen, sulfur, fluorine, and chlorine, such as, for example, ²H, ³H, ¹³C, ¹⁴C, ¹⁵N, ¹⁸O, ¹⁷O, ³⁵S, ¹⁸F, ³⁶Cl. In one aspect, isotopically-labeled lipids described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements.

In some embodiments, the asymmetric carbon atom of the amino lipid is present in enantiomerically enriched form. In certain embodiments, the asymmetric carbon atom of the amino lipid has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the(S)- or (R)-configuration.

In some embodiments, the disclosed amino lipids can be converted to N-oxides. In some embodiments, N-oxides are formed by a treatment with an oxidizing agent (e.g., 3-chloroperoxybenzoic acid and/or hydrogen peroxides). Accordingly, disclosed herein are N-oxide compounds of the described amino lipids, when allowed by valency and structure, which can be designated as NO or N⁺—O⁻. In some embodiments, the nitrogen in the compounds of the disclosure can be converted to N-hydroxy or N-alkoxy. For example, N-hydroxy compounds can be prepared by oxidation of the parent amine by an oxidizing agent such as ra-CPBA. All shown nitrogen-containing compounds are also considered. Accordingly, also disclosed herein are N-hydroxy and N-alkoxy (e.g., N—OR, wherein R is substituted or unsubstituted C₁-C₆ alkyl, C₁-C₆ alkenyl, C₁-C₆ alkynyl, 3-14-membered carbocycle or 3-14-membered heterocycle) derivatives of the described amino lipids.

In some embodiments, the one or more amino lipids comprise from about 40 mol % to about 65 mol % of the total lipid present in the particle.

### PEG-Lipids

In some embodiments, the described LNP composition includes one or more PEG-lipids. As used herein, a “PEG lipid” or “PEG-lipid” refers to a lipid comprising a polyethylene glycol component. Examples of suitable PEG-lipids also include, but are not limited to, PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, the one or more PEG-lipids can comprise PEG-c-DOMG, PEG-DMG, PEG-DLPE, PEG-DMPE, PEG-DPPC, a PEG-DSPE lipid, or a combination thereof.

In some embodiments, PEG-lipid comprises from about 0.1 mol % to about 10 mol % of the total lipid present in the particle.

### Phospholipid

In some embodiments, the described LNP composition includes one or more phospholipids.

In some embodiments, the phospholipid comprises from about 5 mol % to about 15 mol % of the total lipid present in the particle.

### Cholesterol

In some embodiments, the LNP composition includes a cholesterol or a derivative thereof.

### GalNAc-Lipid

In some embodiments, the LNP composition includes a receptor targeting conjugate comprising a compound formula (V),

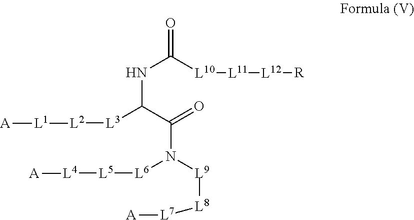

wherein,
a plurality of the A groups collectively comprise a receptor targeting ligand;
each L¹, L², L³, L⁴, L⁵, L⁶, L⁷, L⁸, L⁹, L¹⁰ and L¹² is independently substituted or unsubstituted C₁-C₁₂ alkylene, substituted or unsubstituted C₁-C₁₂ heteroalkylene, substituted or unsubstituted C₂-C₁₂ alkenylene, substituted or unsubstituted C₂-C₁₂ alkynylene, —(CH₂CH₂O)ₘ—, —(OCH2CH₂)ₘ—, —O—, —S—, —S(═O)—, —S(═O)₂—, —S(═O) (═NR¹)—, —C(═O)—, —C(═N—OR¹)—, —C(═O)O—, —OC(═O)—, —C(═O)C(═O)—, —C(═O)NR¹—, —NR¹C(═O)—, —OC(═O)NR¹—, —NR¹C(═O)O—, —NR¹C(═O)NR¹—, —C(═O)NR¹C(═O)—, —S(═O)₂NR¹—, —NR¹S(═O)₂—, —NR¹—, or —N(OR¹)—;
L¹¹ is substituted or unsubstituted —(CH₂CH₂O)ₙ—, substituted or unsubstituted —(OCH₂CH₂)ₙ— or substituted or unsubstituted —(CH₂)ₙ—;
each R¹ is independently H or substituted or unsubstituted C₁-C₆alkyl;
R is a lipid, nucleic acid, amino acid, protein, or lipid nanoparticle;
m is an integer selected from 1 to 10; and
n is an integer selected from 1 to 200.

In some embodiments, each L¹, L⁴, and L⁷ is independently substituted or unsubstituted C₁-C₁₂ alkylene. In some embodiments, each L¹, L⁴, and L⁷ is independently substituted or unsubstituted C₂-C₆ alkylene. In some embodiments, each L¹, L⁴, and L⁷ is C₄ alkylene. In some embodiments, each L², L⁵, and L⁸ is independently —C(═O)NR¹—, —NR¹C(═O)—, —OC(═O)NR¹—, —NR¹C(═O)O—, —NR¹C(═O)NR¹—, or —C(═O)NR¹C(═O)—. In some embodiments, each L², L⁵, and L⁸ is independently-C(═O)NR¹— or —NR¹C(═O)—. In some embodiments, each L², L⁵, and L⁸ is —C(═O) NH—. In some embodiments, each L³, L⁶, and L⁹ is independently substituted or unsubstituted C₁-C₁₂ alkylene. In some embodiments, each L³ is substituted or unsubstituted C₂-C₆ alkylene. In some embodiments, L³ is C₄ alkylene. In some embodiments, each L⁶ and L′ is independently substituted or unsubstituted C₂-C₁₀ alkylene. In some embodiments, each L⁶ and L′ is independently substituted or unsubstituted C₂-C₆ alkylene. In some embodiments, each L⁶ and L′ is C₃ alkylene. In some embodiments, A binds to a lectin. In some embodiments, the lectin is an asialoglycoprotein receptor (ASGPR). In some embodiments, A is N-acetylgalactosamine (GalNAc) or

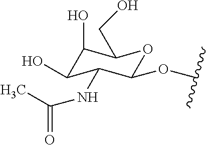 [SMILES: *OC1OC(CO)C(O)C(O)C1NC(C)=O]

or a derivative thereof. A is N-acetylgalactosamine (GalNAc)

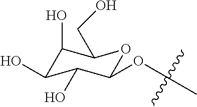 [SMILES: *OC1OC(CO)C(O)C(O)C1O]

or a derivative thereof.

In some embodiments, the receptor targeting conjugate comprises from about 0.001 mol % to about 20 mol % of the total lipid content present in the nanoparticle composition.

### Phosphate Charge Neutralizer

In some embodiments, the LNP described herein includes a phosphate charge neutralizer. In some embodiments, the phosphate charge neutralizer comprises arginine, asparagine, glutamine, lysine, histidine, cationic dendrimers, polyamines, or a combination thereof. In some embodiments, the phosphate charge neutralizer comprises one or more nitrogen atoms. In some embodiments, the phosphate charge neutralizer comprises a polyamine.

Suitable phosphate charge neutralizers to be used in LNP formulations, set forth below, for example include, but are not limited to, Spermidine and 1,3-propanediamine.

### Antioxidants

In some embodiments, the LNP described herein includes one or more antioxidants. In some embodiments, the one or more antioxidants function to reduce a degradation of the cationic lipids, the payload, or both. In some embodiments, the one or more antioxidants comprise a hydrophilic antioxidant. In some embodiments, the one or more antioxidants is a chelating agent such as ethylenediaminetetraacetic acid (EDTA) and citrate. In some embodiments, the one or more antioxidants comprise a lipophilic antioxidant. In some embodiments, the lipophilic antioxidant comprises a vitamin E isomer or a polyphenol. In some embodiments, the one or more antioxidants are present in the LNP composition at a concentration of at least 1 mM, at least 10 mM, at least 20 mM, at least 50 mM, or at least 100 mM. In some embodiments, the one or more antioxidants are present in the particle at a concentration of about 20 mM.

### Other Lipids

In some embodiments, the disclosed LNP compositions may comprise a helper lipid. In some embodiments, the disclosed LNP compositions comprise a neutral lipid. In some embodiments, the disclosed LNP compositions comprise a stealth lipid. In some embodiments, the disclosed LNP compositions comprises additional lipids. Neutral lipids can function to stabilize and improve processing of the LNPs.

“Helper lipids” can refer to lipids that enhance transfection (e.g., transfection of the nanoparticle (LNP) comprising the composition as provided herein, including the biologically active agent). The mechanism by which the helper lipid enhances transfection includes enhancing particle stability. In some embodiments, the helper lipid enhances membrane fusogenicity. Helper lipids can include steroids, sterols, and alkyl resorcinols. Helper lipids suitable for use in the present disclosure can include, but are not limited to, cholesterol, 5-heptadecylresorcinol, and cholesterol hemisuccinate. In some embodiments, the helper lipid is cholesterol. In some embodiments, the helper lipid may be cholesterol hemisuccinate.

“Stealth lipids” can refer to lipids that alter the length of time the nanoparticles can exist in vivo (e.g., in the blood). Stealth lipids can assist in the formulation process by, for example, reducing particle aggregation and controlling particle size. Stealth lipids used herein may modulate pharmacokinetic properties of the LNP. Stealth lipids suitable for use in a lipid composition of the disclosure can include, but are not limited to, stealth lipids having a hydrophilic head group linked to a lipid moiety. Stealth lipids suitable for use in a lipid composition of the present disclosure and information about the biochemistry of such lipids can be found in Romberg et al, Pharmaceutical Research, Vol. 25, No. 1, 2008, pg. 55-71 and I-Toekstra et al, Biochimica et Biophysica Acta 1660 (2004) 41-52. Additional suitable PEG lipids are disclosed, e.g., in WO 2006/007712.

In some embodiments, the stealth lipid is a PEG-lipid. In one embodiment, the hydrophilic head group of stealth lipid comprises a polymer moiety selected from polymers based on PEG (sometimes referred to as poly (ethylene oxide)), poly (oxazoline), poly(vinyl alcohol), poly(glycerol), poly(N-vinylpyrrolidone), polyaminoacids and poly N-(2-hydroxypropyl)methacrylamide]. Stealth lipids can comprise a lipid moiety. In some embodiments, the lipid moiety of the stealth lipid may be derived from diacylglycerol or diacylglycamide, including those comprising a dialkylglycerol or dialkylglycamide group having alkyl chain length independently comprising from about C4 to about C40 saturated or unsaturated carbon atoms, wherein the chain may comprise one or more functional groups such as, for example, an amide or ester. The dialkylglycerol or dialkylglycamide group can further comprise one or more substituted alkyl groups.

The structures and properties of helper lipids, neutral lipids, stealth lipids, and/or other lipids are further described in WO2017173054A1, WO2019067999A1, US20180290965A1, US20180147298A1, US20160375134A1, U.S. Pat. Nos. 8,236,770, 8,021,686, 8,236,770B2, U.S. Pat. No. 7,371,404B2, U.S. Pat. No. 7,780,983B2, U.S. Pat. No. 7,858,117B2, US20180200186A1, US20070087045A1, WO2018119514A1, and WO2019067992A1, all of which are hereby incorporated by reference in their entirety.

### LNP Formulations

Particular formulation of a nanoparticle composition comprising one or more described lipids is described herein.

The described nanoparticle compositions are capable of delivering a therapeutic agent such as an RNA to a particular cell, tissue, organ, or system or group thereof in a mammal's body. Physiochemical properties of nanoparticle compositions may be altered in order to increase selectivity for particular bodily targets. For instance, particle sizes may be adjusted based on the fenestration sizes of different organs. The therapeutic agent included in a nanoparticle composition may also be selected based on the desired delivery target or targets. For example, a therapeutic agent may be selected for a particular indication, condition, disease, or disorder and/or for delivery to a particular cell, tissue, organ, or system or group thereof (e.g., localized, or specific delivery). In certain embodiments, a nanoparticle composition may include an mRNA encoding a polypeptide of interest capable of being translated within a cell to produce the polypeptide (e.g., base editor) of interest. Such a composition are capable of having specificity or affinity to a particular organ or cell type to facilitate drug substance delivery thereto, for example the liver or hepatocytes.

The amount of a therapeutic agent or drug substance (e.g., the mRNA that encodes for the base editor and the guide RNA) in an LNP composition may depend on the size, composition, desired target and/or application, or other properties of the nanoparticle composition. For example, the amount of an RNA comprised in a nanoparticle composition may depend on the size, sequence, and other characteristics of the RNA. The relative amounts of a therapeutic agent and other elements (e.g., lipids) in a nanoparticle composition may also vary. In some embodiments, the wt/wt ratio of the lipid component to a therapeutic agent in a nanoparticle composition may be from about 5:1 to about 60:1, such as about 5:1. 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, and 60:1. For example, the wt/wt ratio of the lipid component to a therapeutic agent may be from about 10:1 to about 40:1. In certain embodiments, the wt/wt ratio is about 20:1. The amount of a therapeutic agent in a nanoparticle composition can be measured using absorption spectroscopy (e.g., ultraviolet-visible spectroscopy).

In some embodiments, an LNP formulation comprises one or more nucleic acids such as RNAs. In some embodiments, the one or more RNAs, lipids, and amounts thereof may be selected to provide a specific N/P ratio. The N/P ratio can be selected from about 1 to about 30. The N/P ratio can be selected from about 2 to about 12. In some embodiments, the N/P ratio is from about 0.1 to about 50. In some embodiments, the N/P ratio is from about 2 to about 8. In some embodiments, the N/P ratio is from about 2 to about 15, from about 2 to about 10, from about 2 to about 8, from about 2 to about 6, from about 3 to about 15, from about 3 to about 10, from about 3 to about 8, from about 3 to about 6, from about 4 to about 15, from about 4 to about 10, from about 4 to about 8, or from about 4 to about 6. In some embodiments, the N/P ratio is about 2, about 2.5, about 3, about 3.5, about 4, about 4.5, about 5, about 5.5, about 6, about 6.5, about 7, about 7.5, about 8, about 9, or about 10. In some embodiments, the N/P ratio is from about 4 to about 6. In some embodiments, the NIP ratio is about 4, about 4.5, about 5, about 5.5, or about 6.

As used herein, the “N/P ratio” is the molar ratio of ionizable (e.g., in the physiological pH range) nitrogen atoms in a lipid (or lipids) to phosphate groups in a nucleic acid molecular entity (or nucleic acid molecular entities), e.g., in a nanoparticle composition comprising a lipid component and an RNA. Ionizable nitrogen atoms can include, for example, nitrogen atoms that can be protonated at about pH 1, about pH 2, about pH 3, about pH 4, about pH 4.5, about pH 5, about pH 5.5, about pH 6, about pH 6.5, about pH 7, about pH 7.5, or about pH 8 or higher. The physiological pH range can include, for example, the pH range of different cellular compartments (such as organs, tissues, and cells) and bodily fluids (such as blood, CSF, gastric juice, milk, bile, saliva, tears, and urine). In certain specific embodiments, the physiological pH range refers to the pH range of blood in a mammal, for example, from about 7.35 to about 7.45. Similarly, for phosphate charge neutralizers that have one or more ionizable nitrogen atoms, the N/P ratio can refer to a molar ratio of ionizable nitrogen atoms in the phosphate charge neutralizer to the phosphate groups in a nucleic acid. In some embodiments, ionizable nitrogen atoms refer to those nitrogen atoms that are ionizable within a pH range between 5 and 14.

For the payload that does not contain a phosphate group, the N/P ratio can refer to a molar ratio of ionizable nitrogen atoms in a lipid to the total negative charge in the payload. For example, the N/P ratio of an LNP composition can refer to a molar ratio of the total ionizable nitrogen atoms in the LNP composition to the total negative charge in the payload that is present in the composition.

In some embodiments, the LNPs are formed with an average encapsulation efficiency ranging from about 50% to about 70%, from about 70% to about 90%, or from about 90% to about 100%. In some embodiments, the LNPs are formed with an average encapsulation efficiency ranging from about 75% to about 98%.

In another aspect, provided herein is a lipid nanoparticle (LNP) comprising the composition as provided herein. As used herein, a “lipid nanoparticle (LNP) composition” or a “nanoparticle composition” is a composition comprising one or more described lipids. LNP compositions are typically sized on the order of micrometers or smaller and may include a lipid bilayer. Nanoparticle compositions encompass lipid nanoparticles (LNPs), liposomes (e.g., lipid vesicles), and lipoplexes. In some embodiments, a LNP refers to any particle that has a diameter of less than 1000 nm, 500 nm, 250 nm, 200 nm, 150 nm, 100 nm, 75 nm, 50 nm, or 25 nm. In some embodiments, a nanoparticle may range in size from 1-1000 nm, 1-500 nm, 1-250 nm, 25-200 nm, 40-100 nm, 50-100 nm. 50-90 nm, 50-80 nm, 50-70 nm, 55-95 nm, 55-80 nm, 55-75 nm, 60-100 nm, 60-90 nm, 60-80 nm, 60-70 nm, 25-100 nm, 25-80 nm, or 40-80 nm.

In some embodiments, an LNP may be made from cationic, anionic, or neutral lipids. In some embodiments, an LNP may comprise neutral lipids, such as the fusogenic phospholipid 1,2-dioleoyl-sn-glycero-3-phosphoethanolamine (DOPE) or the membrane component cholesterol, as helper lipids to enhance transfection activity and nanoparticle stability. In some embodiments, an LNP may comprise hydrophobic lipids, hydrophilic lipids, or both hydrophobic and hydrophilic lipids. Any lipid or combination of lipids that are known in the art can be used to produce an LNP. Examples of lipids used to produce LNPs include, but are not limited to DOTMA (N [1-(2,3-dioleyloxy) propyl]-N,N,N-trimethylammonium chloride), DOSPA (N,N-dimethyl-N-([2-sperminecarboxamido] ethyl)-2,3-bis (dioleyloxy)-1-propaniminium pentahydrochloride), DOTAP(1,2-Dioleoyl-3-trimethylammonium propane), DMRIE (N-(2-hydroxyethyl)-N,N-dimethyl-2,3-bis (tetradecyloxy-1-propanaminiumbromide), DC-cholesterol (3B-[N-(N′,N′-dimethylaminoethane)-carbamoyl] cholesterol), DOTAP-cholesterol, GAP-DMORIE-DPyPE, and GL67A-DOPE-DMPE 1,2-Bis (dimethylphosphino) ethane)-polyethylene glycol (PEG). Examples of cationic lipids include, but are not limited to, 98N12-5, C12-200, DLin-KC2-DMA (KC2), DLin-MC3-DMA (MC3), XTC, MD1, and 7C1. Examples of neutral lipids include, but are not limited to, DPSC, DPPC(Dipalmitoylphosphatidylcholine), POPC(1-palmitoyl-2-oleoyl-sn-glycero-3-phosphocholine), DOPE, and SM (sphingomyelin). Examples of PEG-modified lipids include, but are not limited to, PEG-DMG (Dimyristoyl glycerol), PEG-CerC14, and PEG-CerC20. In some embodiments, the lipids may be combined in any number of molar ratios to produce an LNP. In some embodiments, the polynucleotide may be combined with lipid(s) in a wide range of molar ratios to produce an LNP.

The definitions of terms in the following eight paragraphs apply only the compounds of exemplary lipids described in WO2022140252, WO2022159472, WO 2021141969, WO2021113365, WO2022140239, WO2022140238, WO2022159421, WO2022159475, and WO2022159463 above.

The term “substituted”, unless otherwise indicated, refers to the replacement of one or more hydrogen radicals in a given structure with the radical of a specified substituent including, but not limited to: halo, alkyl, alkenyl, alkynyl, aryl, heterocyclyl, thiol, alkylthio, oxo, thioxy, arylthio, alkylthioalkyl, arylthioallcyl, alkylsulfonyl, alkylsulfonylalkyl, arylsulfonylalkyl, alkoxy, aryloxy, aralkoxy, aminocarbonyl, alkylaminocarbonyl, aiylaminocarbonyl, alkoxycarbonyl, aryloxycarbonyl, haloalkyl, amino, trifluoromethyl, cyano, nitro, alkylamino, arylamino, alkylaminoalkyl, arylaminoalkyl, aminoalkylamino, hydroxy, alkoxyalkyl, carboxyalkyl, alkoxycarbonylalkyl, aminocarbonylalkyl, acyl, aralkoxycarbonyl, carboxylic acid, sulfonic acid, sulfonyl, phosphonic acid, aryl, heteroaryl, heterocyclic, and an aliphatic group. It is understood that the substituent may be further substituted. Exemplary substituents include amino, alkylamino, and the like.

As used herein, the term “substituent” means positional variables on the atoms of a core molecule that are substituted at a designated atom position, replacing one or more hydrogens on the designated atom, provided that the designated atom's normal valency is not exceeded, and that the substitution results in a stable compound. Combinations of substituents and/or variables are permissible only if such combinations result in stable compounds. A person of ordinary skill in the art should note that any carbon as well as heteroatom with valences that appear to be unsatisfied as described or shown herein is assumed to have a sufficient number of hydrogen atom(s) to satisfy the valences described or shown. In certain instances, one or more substituents having a double bond (e.g., “oxo” or “═O”) as the point of attachment may be described, shown, or listed herein within a substituent group, wherein the structure may only show a single bond as the point of attachment to the core structure of Formula (I). A person of ordinary skill in the art would understand that, while only a single bond is shown, a double bond is intended for those substituents.

The term “alkyl” refers to a straight or branched hydrocarbon chain radical, having from one to twenty carbon atoms, and which is attached to the rest of the molecule by a single bond. An alkyl comprising up to 10 carbon atoms is referred to as a C₁-C₁₀ alkyl, likewise, for example, an alkyl comprising up to 6 carbon atoms is a C₁-C₆ alkyl. Alkyls (and other moieties defined herein) comprising other numbers of carbon atoms are represented similarly. Alkyl groups include, but are not limited to, C₁-C₁₀ alkyl, C₁-C₉ alkyl, C₁-C₈ alkyl. C₁-C₇ alkyl, C₁-C₆ alkyl, C₁-C₈alkyl, C₁-C₄ alkyl, C₁-C₃ alkyl, C₁-C₂ alkyl, C₂-C₅ alkyl, C₃-C₈ alkyl and C₄-C₈ alkyl. Representative alkyl groups include, but are not limited to, methyl, ethyl, n-propyl, 1-methylethyl (i-propyl), n-butyl, i-butyl, s-butyl, n-pentyl, 1,1-dimethylethyl (t-butyl), 3-methylhexyl, 2-methylhexyl, 1-ethyl-propyl, and the like. In some embodiments, the alkyl is methyl or ethyl. In some embodiments, the alkyl is —CH (CH₃)₂ or —C(CH₃)₃. Unless stated otherwise specifically in the specification, an alkyl group may be optionally substituted as described below. “Alkylene” or “alkylene chain” refers to a straight or branched divalent hydrocarbon chain linking the rest of the molecule to a radical group. In some embodiments, the alkylene is —CI-12—, —CH₂CH₂—, or —CH₂CH₂CH₂—. In some embodiments, the alkylene is —CH₂—. In some embodiments, the alkylene is —CH₂CH₂—. In some embodiments, the alkylene is —CH₂CH₂CH₂—.

The term “alkenyl” refers to a type of alkyl group in which at least one carbon-carbon double bond is present. In one embodiment, an alkenyl group has the formula —C(R)═CR², wherein R refers to the remaining portions of the alkenyl group, which may be the same or different. In some embodiments, R is H or an alkyl. In some embodiments, an alkenyl is selected from ethenyl (i.e., vinyl), propenyl (i.e., allyl), butenyl, pentenyl, pentadienyl, and the like. Non-limiting examples of an alkenyl group include —CH═CH₂, —C(CH₃)═CH₂, —CH═CHCH₃, —C(CH₃)═CHCH₃, and —CH₂CH═CH₂.

The term “cycloalkyl” refers to a monocyclic or polycyclic non-aromatic radical, wherein each of the atoms forming the ring (i.e., skeletal atoms) is a carbon atom. In some embodiments, cycloalkyls are saturated or partially unsaturated. In some embodiments, cycloalkyls are spirocyclic or bridged compounds. In some embodiments, cycloalkyls are fused with an aromatic ring (in which case the cycloalkyl is bonded through a non-aromatic ring carbon atom). Cycloalkyl groups include groups having from 3 to 10 ring atoms. Representative cycloalkyls include, but are not limited to, cycloalkyls having from three to ten carbon atoms, from three to eight carbon atoms, from three to six carbon atoms, or from three to five carbon atoms. Monocyclic cycloalkyl radicals include, for example, cyclopropyl, cyclobutyl, cyclopentyl, cyclohexyl, cycloheptyl, and cyclooctyl. In some embodiments, the monocyclic cycloalkyl is cyclopropyl, cyclobutyl, cyclopentyl or cyclohexyl. In some embodiments, the monocyclic cycloalkyl is cyclopentenyl or cyclohexenyl. In some embodiments, the monocyclic cycloalkyl is cyclopenteny 1. Polycyclic radicals include, for example, adamantyl, 1,2-dihydronaphthalenyl, 1,4-dihydronaphthalenyl, tetrainyl, decalinyl, 3,4-dihydronaphthalenyl-. 1 (2H)-one. spiro[2.2] pentyl, norbomyl and bicycle [1.1.1]pentyl. Unless otherwise stated specifically in the specification, a cycloalkyl group may be optionally substituted. Depending on the structure, a cycloalkyl group can be monovalent or divalent (i.e., a cycloalkylene group).

The term “heterocycle” or “heterocyclic” refers to heteroaromatic rings (also known as heteroaryls) and heterocycloalkyls (also known as heteroalicyclic groups) that includes at least one heteroatom selected from nitrogen, oxygen, and sulfur, wherein each heterocyclic group has from 3 to 12 atoms in its ring system, and with the proviso that any ring does not contain two adjacent O or S atoms. A “heterocyclyl” is a univalent group formed by removing a hydrogen atom from any ring atoms of a heterocyclic compound. In some embodiments, heterocycles are monocyclic, bicyclic, polycyclic, spirocyclic or bridged compounds. Non-aromatic heterocyclic groups (also known as heterocycloalkyls) include rings having 3 to 12 atoms in its ring system and aromatic heterocyclic groups include rings having 5 to 12 atoms in its ring system. The heterocyclic groups include benzofused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4Hpyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazoli diny I, 3-az.abicy cl o [3. 1.0]hexany 1,3-azabicyclo[4.1.0]heptanyl, 3 h-indolyl, indolin-2-onyl, isoindolin-1-onyl, isoindoline-1,3-dionyl, 3,4-dihydroisoquinolin-1 (2H)-onyl, 3,4-dihydroquinolin-2 (1H)-onyl, isoindoline-1,3-dithionyl, benzo[d] oxazol-2 (3H)-onyl, 1H-benzo[d] imidazol-2 (3H)-onyl, benzo[d] thiazol-2 (3H)-onyl, and quinolizinyl. Examples of aromatic heterocyclic groups are pyridinyl, imidazolyl, pyrimidinyl, pyrazolyl, triazolyl, pyrazinyl, tetrazolyl, futyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furaz.anyl, benzofuraz.anyl, benzothiophenyl, benzothiazolyl, benzoxazolyl, quinazolinyl, quinoxalinyl, naphthyridinyl, and furopyridinyl. The foregoing groups are either C-attached (or Clinked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic.

The term “heterocycloalkyl” refers to a cycloalkyl group that includes at least one heteroatom selected from nitrogen, oxygen, and sulfur. Unless stated otherwise specifically in the specification, the heterocycloalkyl radical may be a monocyclic, or bicyclic ring system, which may include fused (when fused with an aryl or a heterowyl ring, the heterocycloalkyl is bonded through a non-aromatic ring atom) or bridged ring systems. The nitrogen, carbon, or sulfur atoms in the heterocyclyl radical may be optionally oxidized. The nitrogen atom may be optionally quaternized. The heterocycloalkyl radical is partially or fully saturated. Examples of heterocycloalkyl radicals include, but are not limited to, dioxolanyl, thienyl[1,3]dithianyl, tetrahydroquinolyl, tetrahydroisoquinolyl, decahydroquinolyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxothiomorpholinyl, 1,1-dioxo-thiomorpholinyl. The term heterocycloalkyl also includes all ring forms of carbohydrates, including but not limited to monosaccharides, disaccharides, and oligosaccharides. Unless otherwise noted, heterocycloalkyls have from 2 to 12 carbons in the ring. In some embodiments, heterocycloalkyls have from 2 to 10 carbons in the ring. In some embodiments, heterocycloalkyls have from 2 to 10 carbons in the ring and 1 or 2 N atoms. In some embodiments, heterocycloalkyls have from 2 to 10 carbons in the ring and 3 or 4 N atoms. In some embodiments, heterocycloalkyls have from 2 to 12 carbons, 0-2 N atoms, 0-2 O atoms, 0-2 P atoms, and 0-1 S atoms in the ring. In some embodiments, heterocycloalkyls have from 2 to 12 carbons, 1-3 N atoms, 0-1 O atoms, and 0-1 S atoms in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e., skeletal atoms of the heterocycloalkyl ring). Unless stated otherwise specifically in the specification, a heterocycloalkyl group may be optionally substituted. As used herein, the term “teterocycloalkylene” can refer to a divalent heterocycloalkyl group.

The term “heteroaryl” refers to an aryl group that includes one or more ring heteroatoms selected from nitrogen, oxygen, and sulfur. The heteroaryl is monocyclic or bicyclic. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, furazanyl, indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Illustrative examples of bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, heteroaryl is pyridinyl, pyrazinyl, pyrimidinyl, thiazolyl, thienyl, thiadiazolyl or furyl. In some embodiments, a heteroaryl contains 0-6 N atoms in the ring. In some embodiments, a heteroaryl contains 1˜4 N atoms in the ring. In some embodiments, a heteroaryl contains 4-6 N atoms in the ring. In some embodiments, a heteroaryl contains 0-4 N atoms, 0-1 O atoms, 0-1 P atoms, and 0-1 S atoms in the ring. In some embodiments, a heteroaryl contains 1˜4 N atoms, 0-1 O atoms, and 0-1 S atoms in the ring. In some embodiments, heteroaryl is a C₁-C₉ heteroaryl. In some embodiments, monocyclic heteroaryl is a C₁-C₅ heteroaryl. In some embodiments, monocyclic heteroaryl is a 5-membered or 6-membered heteroaryl. In some embodiments, a bicyclic heteroaryl is a C₆-C₉ heteroaryl. In some embodiments, a heteroaryl group is partially reduced to form a heterocycloalkyl group defined herein. In some embodiments, a heteroaryl group is fully reduced to form a heterocycloalkyl group defined herein.

The definitions of terms in the following twenty-five paragraphs apply only the compounds of Formula A′, III-a, and III-a-I, 1, VI, and VIA above

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “carbocyclic”, “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 carbon atoms. In some embodiments, aliphatic groups contain 1-4 carbon atoms. In some embodiments, aliphatic groups contain 1-3 carbon atoms, and in some embodiments, aliphatic groups contain 1-2 carbon atoms. In some embodiments, “carbocyclic” (or “cycloaliphatic” or “carbocycle” or “cycloalkyl”) refers to an optionally substituted monocyclic C₃-C₈ hydrocarbon, or an optionally substituted C₆-C₁₂ bicyclic hydrocarbon, that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl) alkenyl.

As used herein, the term “alkenyl” refers to an alkyl group, as defined herein, having one or more double bonds. In some embodiments, the term “alkenyl”, used alone or as part of a larger moiety, refers to an optionally substituted straight or branched hydrocarbon chain having at least one double bond and having (unless otherwise specified)₂-20, 2-18, 2-16, 2-14, 2-12, 2-10, 2-8, 2-6, 2-4, or 2-3 carbon atoms (e.g., C₂₋₂₀, C₂₋₁₈, C₂₋₁₆, C₂₋₁₄, C₂₋₁₂, C₂₋₁₀, C₂₋₈, C₂₋₆, C₂₋₄, or C₂₋₃). Exemplary alkenyl groups include ethenyl, propenyl, butenyl, pentenyl, hexenyl, and heptenyl.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

As used herein, the term “alkyl” is given its ordinary meaning in the art and may include saturated aliphatic groups, including straight-chain alkyl groups, branched-chain alkyl groups, cycloalkyl (alicyclic) groups, alkyl substituted cycloalkyl groups, and cycloalkyl substituted alkyl groups. In some embodiments, alkyl has 1-100 carbon atoms. In certain embodiments, a straight chain or branched chain alkyl has about 1-20 carbon atoms in its backbone (e.g., C₁-C₂₀ for straight chain, C₂-C₂₀ for branched chain), and alternatively, about 1-10. In some embodiments, a cycloalkyl ring has from about 3-10 carbon atoms in their ring structure where such rings are monocyclic or bicyclic, and alternatively about 5, 6 or 7 carbons in the ring structure. In some embodiments, an alkyl group may be a lower alkyl group, wherein a lower alkyl group comprises 1˜4 carbon atoms (e.g., C₁-C₄ for straight chain lower alkyls).

The term “alkylenyl” or “alkylene” refers to a bivalent alkyl group (i.e., a bivalent saturated hydrocarbon chain) that is a straight-chain (i.e., unbranched) or branched, substituted or unsubstituted. Any of the above mentioned monovalent alkyl groups may be an alkylenyl by abstraction of a second hydrogen atom from the alkyl. In some embodiments, an “alkylenyl” is a polymethylene group, i.e., —(CH₂)ₙ—, wherein n is a positive integer, preferably from 1 to 10, from 1 to 9, from 1 to 8, from 1 to 7, from 1 to 6, from 1 to 5, from 1 to 4, from 1 to 3, from 1 to 2, from 2 to 5, or from 4 to 8. A substituted alkylenyl is a polymethylene group in which one or more methylene hydrogen atoms are replaced with a substituent. Suitable substituents include those described below for a substituted aliphatic group.

As used herein, the term “alkynyl” refers to an alkyl group, as defined herein, having one or more triple bonds. In some embodiments, the term “alkynyl”, used alone or as part of a larger moiety, refers to an optionally substituted straight or branched chain hydrocarbon group having at least one triple bond and having (unless otherwise specified)₂-20, 2-18, 2-16, 2-14, 2-12, 2-10, 2-8, 2-6, 2-4, or 2-3 carbon atoms (e.g., C₂₋₂₀, C₂₋₁₈, C₂₋₁₆, C₂₋₁₄, C₂₋₁₂, C₂₋₁₀, C₂₋₈, C₂₋₆, C₂₋₄, or C₂₋₃). Exemplary alkynyl groups include ethynyl, propynyl, butynyl, pentynyl, hexynyl, and heptynyl.

The term “aryl” refers to monocyclic and bicyclic ring systems having a total of six to fourteen ring members (e.g., C₆₋₁₄), wherein at least one ring in the system is aromatic and wherein each ring in the system contains three to seven ring members. The term “aryl” may be used interchangeably with the term “aryl ring”. In some embodiments, “aryl” refers to an aromatic ring system which includes, but is not limited to, phenyl, naphthyl, anthracyl and the like, which may bear one or more substituents. Unless otherwise specified, “aryl” groups are hydrocarbons.

As used herein, the term “bivalent” refers to a chemical moiety with two points of attachment. For example, a “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bridged bicyclics include but are not limited to:

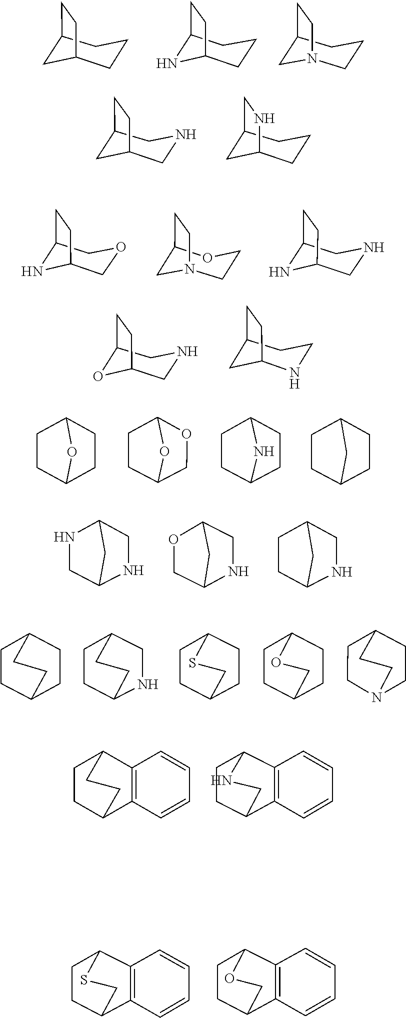

The terms “carbocyclyl,” “carbocycle,” and “carbocyclic ring” as used herein, refer to saturated or partially unsaturated cyclic aliphatic monocyclic, bicyclic, or polycyclic ring systems, as described herein, having from 3 to 14 members, wherein the aliphatic ring system is optionally substituted as described herein. Carbocyclic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, norbornyl, adamantyl, and cyclooctadienyl. In some embodiments, “carbocyclyl” (or “cycloaliphatic”) refers to an optionally substituted monocyclic C₃-C₈ hydrocarbon, or an optionally substituted C₆-C₁₂ bicyclic hydrocarbon that is completely saturated or that contains one or more units of unsaturation, but which is not aromatic, that has a single point of attachment to the rest of the molecule. The term “cycloalkyl” refers to an optionally substituted saturated ring system of about 3 to about 10 ring carbon atoms. In some embodiments, cycloalkyl groups have 3-6 carbons. Exemplary monocyclic cycloalkyl rings include cyclopropyl, cyclobutyl, cyclopentyl, cyclohexyl, and cycloheptyl. The term “cycloalkenyl” refers to an optionally substituted non-aromatic monocyclic or multicyclic ring system containing at least one carbon-carbon double bond and having about 3 to about 10 carbon atoms. Exemplary monocyclic cycloalkenyl rings include cyclopentenyl, cyclohexenyl, and cycloheptenyl.

The term “haloaliphatic” refers to an aliphatic group substituted by one or more halogen atoms (e.g., one, two, three, four, five, six, or seven halo, such as fluoro, iodo, bromo, or chloro). In some embodiments, haloaliphatic groups contain 1-7 halogen atoms. In some embodiments, haloaliphatic groups contain 1-5 halogen atoms. In some embodiments, haloaliphatic groups contain 1-3 halogen atoms.

The term “haloalkyl” refers to an alkyl group substituted by one or more halogen atoms (e.g., one, two, three, four, five, six, or seven halo, such as fluoro, iodo, bromo, or chloro). In some embodiments, haloalkyl groups contain 1-7 halogen atoms. In some embodiments, haloalkyl groups contain 1-5 halogen atoms. In some embodiments, haloalkyl groups contain 1-3 halogen atoms.

The term “heteroalkylenyl” or “heteroalkylene”, as used herein, denotes an optionally substituted straight-chain (i.e., unbranched), or branched bivalent alkyl group (i.e., bivalent saturated hydrocarbon chain) having, in addition to carbon atoms, from one to five heteroatoms. The term “heteroatom” is described below. In some embodiments, heteroalkylenyl groups contain 2-10 carbon atoms wherein 1-3 carbon atoms are optionally and independently replaced with heteroatoms selected from oxygen, nitrogen, and sulfur. In some embodiments, heteroalkylenyl groups contain 2-8 carbon atoms wherein 1-3 carbon atoms are optionally and independently replaced with heteroatoms selected from oxygen, nitrogen, and sulfur. In some embodiments, heteroalkylenyl groups contain 4-8 carbon atoms, wherein 1-3 carbon atoms are optionally and independently replaced with heteroatoms selected from oxygen, nitrogen, and sulfur. In some embodiments, heteroalkylenyl groups contain 2-5 carbon atoms, wherein 1-2 carbon atoms are optionally and independently replaced with heteroatoms selected from oxygen, nitrogen, and sulfur. In yet other embodiments, heteroalkylenyl groups contain 1-3 carbon atoms, wherein 1 carbon atom is optionally and independently replaced with a heteroatom selected from oxygen, nitrogen, and sulfur. Suitable heteroalkylenyl groups include, but are not limited to —CH₂O—, —(CH₂)₂O—, —CH₂OCH₂—, —O(CH₂)₂—, —(CH₂)₃O—, —(CH₂)₂OCH₂—, —CH₂O(CH₂)₂—, —O(CH₂)₃—, —(CH₂)₄O—, —(CH₂)₃OCH₂—, —CH₂O(CH₂)₃—, —(CH₂)₂O(CH₂)₂—, —O(CH₂)₄—. Unless otherwise specified, Cₓ heteroalkylenyl refers to heteroalkylenyl having x number of carbon atoms prior to replacement with heteroatoms.

The terms “heteroaryl” and “heteroar-”, used alone or as part of a larger moiety, e.g., “heteroaralkyl”, or “heteroaralkoxy”, refer to monocyclic or bicyclic ring groups having 5 to 10 ring atoms (e.g., 5- to 6-membered monocyclic heteroaryl or 9- to 10-membered bicyclic heteroaryl); having 6, 10, or 14 π electrons shared in a cyclic array; and having, in addition to carbon atoms, from one to five heteroatoms. Exemplary heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridonyl, pyridazinyl, pyrimidinyl, pyrazinyl, indolizinyl, purinyl, naphthyridinyl, pteridinyl, imidazo[1,2-a]pyrimidinyl, imidazo[1,2-a]pyridinyl, thienopyrimidinyl, triazolopyridinyl, and benzoisoxazolyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring (i.e., a bicyclic heteroaryl ring having 1 to 3 heteroatoms). Nonlimiting examples include indolyl, isoindolyl, benzothienyl, benzofuranyl, dibenzofuranyl, indazolyl, benzimidazolyl, benzothiazolyl, benzothiadiazolyl, benzoxazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, pyrido[2,3-b]-1,4-oxazin-3 (4H)-one, and benzoisoxazolyl. The term “heteroaryl” may be used interchangeably with the terms “heteroaryl ring”, “heteroaryl group”, or “heteroaromatic”, any of which terms include rings that are optionally substituted.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N(as in 3,4-dihydro-2H-pyrrolyl), NH (as in pyrrolidinyl) or NR⁺ (as in N-substituted pyrrolidinyl)).

The terms “heterocycle”, “heterocyclyl”, “heterocyclic radical”, and “heterocyclic ring” are used interchangeably herein, and refer to a stable 3- to 8-membered monocyclic, a 7- to 12-membered bicyclic, or a 10- to 16-membered polycyclic heterocyclic moiety that is either saturated or partially unsaturated, and having, in addition to carbon atoms, one or more, such as one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N(as in 3,4-dihydro-2H-pyrrolyl), NH (as in pyrrolidinyl), or NR⁺ (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, azetidinyl, oxetanyl, tetrahydrofuranyl, tetrahydrothienyl, pyrrolidinyl, piperidinyl, decahydroquinolinyl, oxazolidinyl, piperazinyl, tetrahydropyranyl, dioxanyl, dioxolanyl, diazepinyl, oxazepinyl, thiazepinyl, morpholinyl, thiamorpholinyl, and

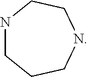

A heterocyclyl group may be mono-, bi-, tri-, or polycyclic, preferably mono-, bi-, or tricyclic, more preferably mono- or bicyclic. The term “heterocyclylalkyl” refers to an alkyl group substituted by a heterocyclyl, wherein the alkyl and heterocyclyl portions independently are optionally substituted. A bicyclic heterocyclic ring also includes groups in which the heterocyclic ring is fused to one or more aryl, heteroaryl, or cycloaliphatic rings. Exemplary bicyclic heterocyclic groups include indolinyl, isoindolinyl, benzodioxolyl, 1,3-dihydroisobenzofuranyl, 2,3-dihydrobenzofuranyl, and tetrahydroquinolinyl. A bicyclic heterocyclic ring can also be a spirocyclic ring system (e.g., 7- to 11-membered spirocyclic fused heterocyclic ring having, in addition to carbon atoms, one or more heteroatoms as defined above (e.g., one, two, three or four heteroatoms)). A bicyclic heterocyclic ring can also be a bridged ring system (e.g., 7- to 11-membered bridged heterocyclic ring having one, two, or three bridging atoms.

As used herein, the term “linker” is used to refer to that portion of a multi-element agent that connects different elements to one another. For example, those of ordinary skill in the art appreciate that a polypeptide whose structure includes two or more functional or organizational domains often includes a stretch of amino acids between such domains that links them to one another. In some embodiments, a polypeptide comprising a linker element “L′” has an overall structure of the general form S1-L′-S2, wherein S1 and S2 may be the same or different and represent two domains associated with one another by the linker. In some embodiments, a polyptide linker is at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100 or more amino acids in length. In some embodiments, a linker is characterized in that it tends not to adopt a rigid three-dimensional structure, but rather provides flexibility to the polypeptide. A variety of different linker elements that can appropriately be used when engineering polypeptides (e.g., fusion polypeptides) known in the art (see e.g., Holliger, P., et al. (1993) Proc. Natl. Acad. Sci. USA 90:6444-6448; Poljak, R. J., et al. (1994) Structure 2:1 121-1123).

The term “sterolyl,” as used herein, refers to a 17-membered fused polycyclic ring moiety that is either saturated or partially unsaturated and substituted with at least one hydroxyl group, and has a single point of attachment to the rest of the molecule at any substitutable carbon or oxygen atom. In some embodiments, a sterolyl group is a cholesterolyl group, or a variant or derivative thereof. In some embodiments, a cholesterolyl group is modified. In some embodiments, a cholesterolyl group is an oxidized cholesterolyl group (e.g., oxidized on the beta-ring structure or on the hydrocarbon tail structure). In some embodiments, a cholesterolyl group is an esterified cholesterolyl group. In some embodiments, a sterolyl group is a phytosterolyl group. Exemplary sterolyl groups include but are not limited to 25-hydroxycholesterolyl (25-OH), 20a-hydroxycholesterolyl (20a-OH), 27-hydroxycholesterolyl, 6-keto-5a-hydroxycholesterolyl, 7-ketocholesterolyl, 7ß-hydroxycholesterolyl, 7a-hydroxycholesterolyl, 7ß-25-dihydroxycholesterolyl, beta-sitosterolyl, stigmasterolyl, brassicasterolyl, and campesterolyl.

As described herein, compounds of this disclosure may be described as “substituted” or “optionally substituted”. That is, compounds may contain optionally substituted and/or substituted moieties. In general, the term “substituted,” whether preceded by the term “optionally” or not, means that one or more hydrogens of the designated moiety are replaced with a suitable substituent. “Substituted” applies to one or more hydrogens that are either explicit or implicit from the structure (e.g.,

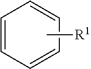

refers to at least

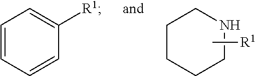

refers to at least

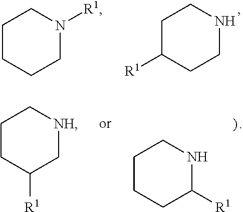

Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein. Groups described as being “substituted” preferably have between 1 and 4 substituents, more preferably 1 or 2 substituents. Groups described as being “optionally substituted” may be unsubstituted or be “substituted” as described above.

Suitable monovalent substituents include halogen; —(CH₂)₀₋₄R^{o}; —(CH₂)₀₋₄OR^{o}; —O(CH₂)₀₋₄R^{o}, —O—(CH₂)₀₋₄C(O)OR^{o})₂; —(CH₂)₀₋₄CH(OR^{o})₂; —(CH₂)₀₋₄Ph, which may be substituted with R^{o}; —(CH₂)₀₋₄O(CH₂)₀₋₁Ph which may be substituted with R^{o}; —CH═CHPh, which may be substituted with R^{o}; —(CH₂)₀₋₄O(CH₂)₀₋₁-pyridyl which may be substituted with R^{o};—NO₂; —CN; —N₃; —(CH₂)₀₋₄N(R^{o})₂; —(CH₂)₀₋₄N(R^{o})C(O)R^{o}; —N(R^{o})C(S)R^{o}; —(CH₂)₀₋₄N(R^{o})C(O)NR°2; —N(R^{o})C(S)NR^{o})₂; —(CH₂)₀₋₄N(R^{o})C(O)OR^{o}; —N(R^{o}N(R^{o})C(O)R^{o}; —N(R^{o}N(R^{o})C(O)NR^{o})₂; —N(R^{o})N(R^{o}C(O)OR^{o}; —(CH₂)₀₋₄C(O)R^{o}; —C(S)R^{o}; —(CH₂)₀₋₄C(O)OR^{o}; —(CH₂)₀₋₄C(O)SR^{o}; —(CH₂)₀₋₄C(O)OSiR^{o}₃; —(CH₂)₀₋₄OC(O)R^{o}; —OC(O)(CH₂)₀₋₄SR^{o}—, —SC(S)SR^{o}; —(CH₂)₀₋₄SC(O)R^{o}; —(CH₂)₀₋₄C(O)NR^{o}₂; —C(S)NR^{o}₂; —C(S)SR^{o}; —SC(S)SR^{o}, —(CH₂)₀₋₄OC(O)NR^{o}₂; —C(O)N(OR^{o})R^{o}; —C(O)C(O)R^{o}; —C(O)CH₂C(O)R^{o}; —C(NOR^{o})R^{o}; —(CH₂)₀₋₄SSR^{o}; —(CH₂)₀₋₄S(O)₂R^{o}; —(CH₂)₀₋₄S(O)₂OR^{o}; —(CH₂)₀₋₄OS(O)₂R^{o}; —S(O)₂NR^{o}₂; —(CH₂)₀₋₄S(O)R^{o}; —N(R^{o})S(O)₂NR^{o}₂; —N(R^{o}S(O)₂R^{o}; —N(OR^{o})R^{o}; —C(NH)NR^{o}₂; —P(O)₂R^{o}; —P(O)R^{o}2; —OP(O)R^{o}2; —)OP(O) (OR^{o}2; —SiR^{o}3; —OSiR^{o}3; —(C₁-4 straight or branched alkylene)O—N(R^{o})₂; or —(C₁-4 straight or branched) alkylene)C(O)O—N(R^{o})₂, wherein each R^{o} may be substituted as defined below and is independently hydrogen, C₁₋₆ aliphatic, —CH₂Ph, —O (CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{o}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{o} (or the ring formed by taking two independent occurrences of R^{o} together with their intervening atoms), are independently halogen, —(CH₂)₀₋₂R^{•}, -(haloR^{•}, —(CH₂)₀₋₂OH, —(CH₂)₀₋₂OR^{•}, —(CH₂)₀₋₂CH (OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂C(O)NH₂, —(CH₂)₀₋₂C(O)NHR^{•}, —(CH₂)₀₋₂C(O)NR^{•}₂, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{o}, or —SSR^{•} wherein each R^{•} is unsubstituted or where preceded by “halo” is substituted only with one or more halogens, and is independently selected from C₁-4 aliphatic, —CH₂Ph, —O(CH₂)₀-1Ph, or a 5-6-membered saturated, partially unsaturated, or aryl ring having 0-4 heteroatoms independently selected from nitrogen, oxygen, or sulfur. Suitable divalent substituents on a saturated carbon atom of R^{o} include ═O and ═S.

Suitable divalent substituents include the following: ═O, ═S, ═NNR*2, ═NNHC(O)R*, ═NNHC(O)OR*,═NNHS(O)₂R*, —NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R*include halogen, —R⁵⁰⁰ , -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

In some embodiments, suitable substituents on a substitutable nitrogen include —R^{†}, —NR^{†}₂, —C(O)R^{†}, —C(O)OR^{†}, —C(O)C(O)R^{†}, —C(O)CH₂C(O)R^{\}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of Rt, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of Rt are independently halogen, —R^{•}, -(haloR^{•}), —OH, —OR^{•}, —O (haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, amino lipids can contain at least one primary, secondary, or tertiary amine moiety that is protonatable (or ionizable) between pH range 4 and 14. In some embodiments, the amine moiety/moieties function as the hydrophilic headgroup of the amino lipids. When most of the amine moiety(ies) of an amino lipid (or amino lipids) in a nucleic acid-lipid nanoparticle formulation is protonated at physiological pH, then the nanoparticles can be termed as cationic lipid nanoparticle (cLNP). When most of the amine moiety(ies) of an amino lipid (or amino lipids) in a nucleic acid-lipid nanoparticle formulation is not protonated at physiological pH but can be protonated at acidic pH, endosomal pH for example, can be termed as ionizable lipid nanoparticle (iLNP). The amino lipids that constitute cLNPs can be generally called cationic amino lipids (cLi pids). The amino lipids that constitute iLNPs can be called ionizable amino lipids (iLipids). The amino lipid can be an iLipid or a cLipid at physiological pH.

As used herein, LNP compositions or formulations are typically sized on the order of micrometers or smaller and may include a lipid bilayer. Nanoparticle compositions encompass lipid nanoparticles (LNPs), liposomes lipid vesicles), and lipoplexesnanoparticle composition a liposome having a lipid bilayer with a diameter of 500 nm or less. The LNPs described herein can have a mean diameter of from about 1 nm to about 2500 nm, from about 10 nm to about 1500 nm, from about 20 nm to about 1000 nm, from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm or from about 70 nm to about 80 nm. The LNPs described herein can have a mean diameter of about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, or greater. The LNPs described herein can be substantially non-toxic.

As used herein, a “phospholipid” can refer to a lipid that includes a phosphate moiety and one or more carbon chains, such as unsaturated fatty acid chains. A phospholipid may include one or more multiple (e.g., double or triple) bonds. In some embodiments, a phospholipid may facilitate fusion to a membrane. For example, a cationic phospholipid may interact with one or more negatively charged phospholipids of a membrane (e.g., a cellular or intracellular membrane). Fusion of a phospholipid to a membrane may allow one or more elements of an LNP to pass through the membrane, i.e., delivery of the one or more elements to a cell.

### Payload

The LNPs described herein can be designed to deliver a payload, such as one or more therapeutic agent(s) or drug substances(s) to a target cell or organ of interest. In some embodiments, a LNP described herein encloses one or more components of a base editor system as described herein. For example, a LNP may enclose one or more of a guide RNA, a nucleic acid encoding the guide RNA, a vector encoding the guide RNA, a base editor fusion protein, a nucleic acid encoding the base editor fusion protein, a programmable DNA binding domain, a nucleic acid encoding the programmable DNA binding domain, a deaminase, a nucleic acid encoding the deaminase, or all or any combination thereof. In some embodiments, the nucleic acid is a DNA. In some embodiments, the nucleic acid is a RNA, for example, a mRNA and/or a guide RNA. In some embodiments, the said nucleic acid(s) is/are chemically modified.

In some embodiments, the payload comprises one or more nucleic acid(s) (i.e., one or more nucleic acid molecular entities). In some embodiments, the nucleic acid is a single-stranded nucleic acid. In some embodiments, single-stranded nucleic acid is a DNA. In some embodiments, single-stranded nucleic acid is an RNA. In some embodiments, the nucleic acid is a double-stranded nucleic acid. In some embodiments, the double-stranded nucleic acid is a DNA. In some embodiments, the double-stranded nucleic acid is an RNA. In some embodiments, the double-stranded nucleic acid is a DNA-RNA hybrid. In some embodiments, the nucleic acid is a messenger RNA (mRNA), a microRNA, an asymmetrical interfering RNA (aiRNA), a small hairpin RNA (shRNA), an antisense oligonucleotide, or a Dicer-Substrate dsRNA. In some embodiments, the single-stranded nucleic acids form secondary structure, one or more stem-loops for example. In some other embodiments, the single stranded nucleic acids contain one or more stem-loops and single-stranded regions within the molecule.

### Non-Viral Platforms for Gene Transfer

Non-viral platforms for introducing a heterologous polynucleotide into a cell of interest are known in the art.

For example, the disclosure provides a method of inserting a heterologous polynucleotide into the genome of a cell using a Cas9 or Cas12 (e.g., Cas12b) ribonucleoprotein complex (RNP)-DNA template complex where an RNP including a Cas9 or Cas12 nuclease domain and a guide RNA, wherein the guide RNA specifically hybridizes to a target region of the genome of the cell, and wherein the Cas nuclease domain cleaves the target region to create an insertion site in the genome of the cell. A DNA template is then used to introduce a heterologous polynucleotide. In embodiments, the DNA template is a double-stranded or single-stranded DNA template, wherein the size of the DNA template is about 200 nucleotides or is greater than about 200 nucleotides, wherein the 5′ and 3′ ends of the DNA template comprise nucleotide sequences that are homologous to genomic sequences flanking the insertion site. In some embodiments, the DNA template is a single-stranded circular DNA template. In embodiments, the molar ratio of RNP to DNA template in the complex is from about 3:1 to about 100:1.

In some embodiments, the DNA template is a linear DNA template. In some examples, the DNA template is a single-stranded DNA template. In certain embodiments, the single-stranded DNA template is a pure single-stranded DNA template. In some embodiments, the single stranded DNA template is a single-stranded oligodeoxynucleotide (ssODN).

In other embodiments, a single-stranded DNA (ssDNA) can produce efficient homology-directed repair (HDR) with minimal off-target integration. In one embodiment, an ssDNA phage is used to efficiently and inexpensively produce long circular ssDNA (cssDNA) donors. These cssDNA donors serve as efficient HDR templates when used with Cas9 or Cas12 (e.g., Cas12a, Cas12b), with integration frequencies superior to linear ssDNA (IssDNA) donors.

In some embodiments, a heterologous polynucleotide may be inserted into the genome of a cell using a transposable element such as a transposon, as described, for example, in Tipanee, et al. Human Gene Therapy, November 2017, 1087-1104, DOI: 10.1089/hum.2017.128. Transposable elements are divided into two categories: retrotransposons and DNA transposons. Transposable elements can alter the genome of the host cells through insertions, duplications, deletions, and translocations. Retrotransposons are described as mobile elements that employ an RNA intermediate that is first reverse transcribed into a complementary single-stranded (c) DNA strand by a reverse transcriptase encoded by the retrotransposon. Subsequently, the single-stranded DNA is converted into a double-stranded DNA that then integrates into the host genome. This so-called “replicative mechanism” yields several new copies of retrotransposons expanding throughout the target genome over evolutionary time. Retrotransposons are categorized into many subtypes according to the DNA sequences of the long terminal repeats and its open reading frames. Retrotransposons were employed to enable transgene integration into the target cell DNA, in some cases relying on adenoviral delivery. Alternatively, DNA transposons translocate via a “non-replicative mechanism,” whereby two Terminal Inverted Repeats (TIRs) are recognized and cleaved by a transposase enzyme, releasing the cognate DNA transposons with free DNA ends. The excised DNA transposons then integrate into a new genomic region where target sites are recognized and cut by the same transposase. This cut-and-paste mechanism usually duplicates DNA target sites upon insertion, leaving target site duplications (TSDs). Non-limiting examples of transposons include the Sleeping Beauty (SB) transposon, the piggyBac (PB) transposon, and Tol2 transposable elements.

### Pharmaceutical Compositions

In some aspects, the present disclosure provides a pharmaceutical composition comprising any of the cells, polynucleotides, vectors, base editors, base editor systems, guide polynucleotides, fusion proteins, complexes, or the fusion protein-guide polynucleotide complexes described herein.

The pharmaceutical compositions of the present disclosure can be prepared in accordance with known techniques. See, e.g., Remington, The Science And Practice of Pharmacy (21st ed. 2005). In general, the cell, or population thereof is admixed with a suitable carrier prior to administration or storage, and in some embodiments, the pharmaceutical composition further comprises a pharmaceutically acceptable carrier. Suitable pharmaceutically acceptable carriers generally comprise inert substances that aid in administering the pharmaceutical composition to a subject, aid in processing the pharmaceutical compositions into deliverable preparations, or aid in storing the pharmaceutical composition prior to administration. Pharmaceutically acceptable carriers can include agents that can stabilize, optimize or otherwise alter the form, consistency, viscosity, pH, pharmacokinetics, solubility of the formulation. Such agents include buffering agents, wetting agents, emulsifying agents, diluents, encapsulating agents, and skin penetration enhancers. For example, carriers can include, but are not limited to, saline, buffered saline, dextrose, arginine, sucrose, water, glycerol, ethanol, sorbitol, dextran, sodium carboxymethyl cellulose, and combinations thereof.

In some embodiments, the pharmaceutical composition is formulated for delivery to a subject. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site (e.g., a liver). In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In some embodiments, any of the fusion proteins, gRNAs, and/or complexes described herein are provided as part of a pharmaceutical composition. In some embodiments, the pharmaceutical composition comprises any of the fusion proteins or complexes provided herein. In some embodiments pharmaceutical composition comprises a gRNA, a nucleic acid programmable DNA binding protein, a cationic lipid, and a pharmaceutically acceptable excipient. In embodiments, pharmaceutical compositions comprise a lipid nanoparticle and a pharmaceutically acceptable excipient. In embodiments, the lipid nanoparticle contains a gRNA, a base editor, a complex, a base editor system, or a component thereof of the present disclosure, and/or one or more polynucleotides encoding the same. Pharmaceutical compositions can optionally comprise one or more additional therapeutically active substances.

The compositions, as described above, can be administered in effective amounts. The effective amount will depend upon the mode of administration, the particular condition being treated, and the desired outcome. It may also depend upon the stage of the condition, the age and physical condition of the subject, the nature of concurrent therapy, if any, and like factors well-known to the medical practitioner. For therapeutic applications, it is that amount sufficient to achieve a medically desirable result.

In some embodiments, compositions in accordance with the present disclosure can be used for treatment of any of a variety of diseases, disorders, and/or conditions.

### Methods of Treatment

Some aspects of the present invention provide methods of treating a subject having or having a propensity to develop amyloidosis, the method comprising administering to a subject in need an effective therapeutic amount of a pharmaceutical composition as described herein. In some embodiments, the methods of the invention comprise expressing or introducing into a cell of a subject a base editor polypeptide and one or more guide RNAs capable of targeting a nucleic acid molecule encoding a transthyretin polypeptide comprising a pathogenic mutation.

One of ordinary skill in the art would recognize that multiple administrations of the pharmaceutical compositions contemplated in particular embodiments may be required to affect the desired therapy. For example, a composition may be administered to the subject 1, 2, 3, 4, 5, 6, 7, 8, 9, or 10 or more times over a span of 1 week, 2 weeks, 3 weeks, 1 month, 2 months, 3 months, 4 months, 5 months, 6 months, 1 year, 2 years, 5, years, 10 years, or more.

In any of such methods, the methods may comprise administering to the subject an effective amount of an edited cell or a base editor system or polynucleotide encoding such system. In any of such methods, the methods may comprise administering one or more doses of an effective amount of the edited cells per day. In any of such methods, the methods may comprise administering two or more doses of an effective amount of the mod edited ified cells per day. In any of such methods, the methods may comprise administering three or more doses of an effective amount of the edited cells per day. In any of such methods, the methods may comprise administering one or more doses of an effective amount of the edited cells per week. In any of such methods, the methods may comprise administering two or more doses of an effective amount of the edited cells per week. In any of such methods, the methods may comprise administering three or more doses of an effective amount of the edited cells per week. In any of such methods, the methods may comprise administering one or more doses of an effective amount of the edited cells per month. In any of such methods, the methods may comprise administering two or more doses of an effective amount of the edited cells per month. In any of such methods, the methods may comprise administering three or more doses of an effective amount of the edited cells per month.

Administration of the pharmaceutical compositions contemplated herein may be carried out using conventional techniques including, but not limited to, infusion, transfusion, or parenterally. In some embodiments, parenteral administration includes infusing or injecting intravascularly, intravenously, intramuscularly, intraarterially, intrathecally, intratumorally, intradermally, intraperitoneally, transtracheally, subcutaneously, subcuticularly, intraarticularly, subcapsularly, subarachnoidly and intrasternally.

In some embodiments, the composition is administered over a period of 0.25 h, 0.5 h, 1 h, 2 h, 3 h, 4 h, 5 h, 6 h, 7 h, 8 h, 9 h, 10 h, 11 h, or 12 h. In another embodiment, the composition is administered over a period of 0.25-2 h. In another embodiment, the composition is gradually administered over a period of 1 h. In another embodiment, the composition is gradually administered over a period of 2 h.

### Kits

The disclosure provides kits for the treatment of amyloidosis in a subject. In some embodiments, the kit further includes a base editor system or a polynucleotide encoding a base editor system, wherein the base editor polypeptide system a nucleic acid programmable DNA binding protein (napDNAbp), a deaminase, and a guide RNA. In some embodiments, the napDNAbp is Cas9 or Cas12. In some embodiments, the polynucleotide encoding the base editor is a mRNA sequence. In some embodiments, the deaminase is a cytidine deaminase or an adenosine deaminase. In some embodiments, the kit comprises an edited cell and instructions regarding the use of such cell.

The kits may further comprise written instructions for using the base editor system and/or edited cell. In other embodiments, the instructions include at least one of the following: precautions; warnings; clinical studies; and/or references. The instructions may be printed directly on the container (when present), or as a label applied to the container, or as a separate sheet, pamphlet, card, or folder supplied in or with the container. In a further embodiment, a kit can comprise instructions in the form of a label or separate insert (package insert) for suitable operational parameters. In yet another embodiment, the kit can comprise one or more containers with appropriate positive and negative controls or control samples, to be used as standard(s) for detection, calibration, or normalization. The kit can further comprise a second container comprising a pharmaceutically-acceptable buffer, such as (sterile) phosphate-buffered saline, Ringer's solution, or dextrose solution. It can further include other materials desirable from a commercial and user standpoint, including other buffers, diluents, filters, needles, syringes, and package inserts with instructions for use.

The practice of embodiments of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the disclosure, and, as such, may be considered in making and practicing embodiments of the disclosure. Particularly useful techniques for particular embodiments will be discussed in the sections that follow.

The following examples are put forth so as to provide those of ordinary skill in the art with a complete disclosure and description of how to make and use the assay, screening, and therapeutic methods of the disclosure, and are not intended to limit the scope of what the inventors regard as their invention.

### EXAMPLE

### Example 1: Guides for Adenine Base Editing of the TTR Gene

In this example, gRNA sequences were identified that permit ABE8.8 (and other ABE variants containing Streptococcus pyogenes Cas9, such as ABE7.10, or another Cas protein that can use the NGG PAM) to either: 1) disrupt the start codon, and/or 2) disrupt splice sites, whether donors or acceptors, via A→G editing within its editing window (roughly positions 4 to 7 in the 20-nt protospacer region of DNA). Five sequences were identified throughout the human TTR gene that disrupt TTR expression (Table 8). gRNAs were synthesized matching each of the protospacer sequences and otherwise conforming to the standard 100-nt Streptococcus pyogenes CRISPR gRNA sequence, with each gRNA molecule having a minimal degree of chemical modifications (specified in Table 8). Each of the gRNAs was co-transfected with an equivalent amount of in vitro transcribed ABE8.8 mRNA (1:1 ratio by molecular weight) into primary human hepatocytes via MessengerMax reagent (Lipofectamine), using various dilutions (2500,1250, 625 ng/RNA/mL) to assess for editing activity at different concentrations of test article.

TABLE 8
TTR Guides

| gRNA | | Protospacer | |
|---|---|---|---|
| ID | Species | (5′-3′) | gRNA sequence (5′-3′) (spacers are in bold) |
| GA457 | Human | GCCATCCT | gscscsAUCCUGCCAAGAAUGAGGUUUUAGAGCUAGA |
| | | GCCAAGAA | AAUAGCAAGUUAAAAUAAGGCUAGUCCGUUAUCAACU |
| | | TGAG (SEQ | UGAAAAAGUGGCACCGAGUCGGUGCUsususu (SEQ |
| | | ID NO: 467) | ID NO: 479) |
| GA519 | Cyno | GCCATCCT | gscscsAUCCUGCCAAGAACGAGgUUUUAGagcuaGa |
| | | GCCAAGAA | aauagcaaGUUaAaAuAaggcuaGUccGUUAucAAcu |
| | | CGAG (SEQ | uGaaaaagugGcaccgagucggugcuususus (SEQ |
| | | ID NO: 471) | ID NO: 1044) |
| GA458 | Cyno | GCCATCCT | gscscsAUCCUGCCAAGAACGAGGUUUUAGAGCUAGA |
| | | GCCAAGAA | AAUAGCAAGUUAAAAUAAGGCUAGUCCGUUAUCAACU |
| | | CGAG (SEQ | UGAAAAAGUGGCACCGAGUCGGUGCUsususu (SEQ |
| | | ID NO: 471) | ID NO: 1045) |
| GA459 | Human | GCAACTTA | gscsasACUUACCCAGAGGCAAAGUUUUAGAGCUAGA |
| | | CCCAGAGG | AAUAGCAAGUUAAAAUAAGGCUAGUCCGUUAUCAACU |
| | | CAAA (SEQ | UGAAAAAGUGGCACCGAGUCGGUGCUsususu (SEQ |
| | | ID NO: 468) | ID NO: 499) |
| GA460 | Human/ | TATAGGAA | usasusAGGAAAACCAGUGAGUCGUUUUAGAGCUAGA |
| | Cyno | AACCAGTG | AAUAGCAAGUUAAAAUAAGGCUAGUCCGUUAUCAACU |
| | | AGTC (SEQ | UGAAAAAGUGGCACCGAGUCGGUGCUsususu (SEQ |
| | | ID NO: 469) | ID NO: 497) |
| GA520 | Human/ | TATAGGAA | usasusAGGAAAACCAGUGAGUCgUUUUAGagcuaGa |
| | Cyno | AACCAGTG | aauagcaaGUUaAaAuAaggcuaGUccGUUAucAAcu |
| | | AGTC (SEQ | uGaaaaagugGcaccgagucggugcuususus (SEQ |
| | | ID NO: 469) | ID NO: 497) |
| GA461 | Human/ | TACTCACC | usascsUCACCUCUGCAUGCUCAGUUUUAGAGCUAGA |
| | Cyno | TCTGCATG | AAUAGCAAGUUAAAAUAAGGCUAGUCCGUUAUCAACU |
| | | CTCA (SEQ | UGAAAAAGUGGCACCGAGUCGGUGCUsususu (SEQ |
| | | ID NO: 470) | ID NO: 480) |
| GA521 | Human | GCCATCCT | mG*mC*mC*ATCCTGCCAAGAATGAGmGUUUUAGmAm |
| | | GCCAAGAA | GmCmUmAGmAmAmAmUmAmGmCmAmAGUUmAAmAAmU |
| | | TGAG (SEQ | AmAmGmGmCmUmAGUmCmCGUUAmUmCAAmCmUmUGm |
| | | ID NO: 467) | AmAmAmAmAmGmUmGGmCmAmCmCmGmAmGmUmCmGm |
| | | | GmUmGmCmU*mU*mU*mU (SEQ ID NO: 1045) |

Letters in the sequences:- A: adenosine; C: cytidine; G: guanosine; U: uridine; a or mA: 2′-O-methyladenosine; c or mC: 2′-O-methylcytidine; g or mG: 2′-O-methylguanosine; u or mU: 2′-O-methyluridine; and s or *: phosphorothioate (PS) backbone linkage. Bold type in gRNA sequence denotes spacer sequence corresponding to Protospacer. GA460 and GA520 have the same protospacer sequence but have different chemical modifications in the gRNA sequence.

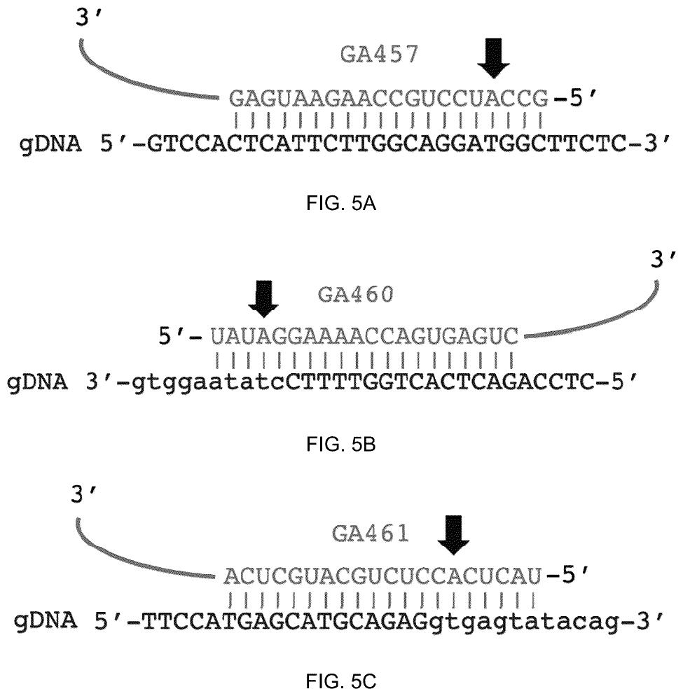

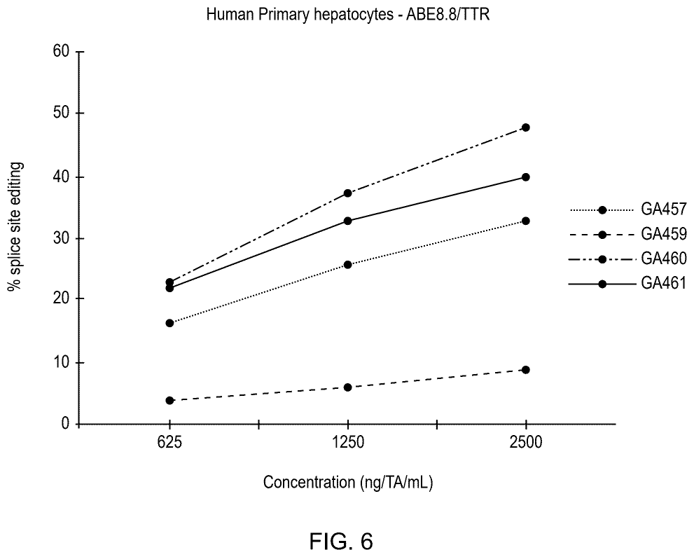

For orthogonal protospacer sequences of the corresponding cynomolgus monkey TTR gene sequence, each gRNA was also transfected with an equivalent amount of ABE8.8 mRNA (1:1 ratio by molecular weight) into primary cynomolgus hepatocytes at 5000, 2500, 1250, 625, 312.5, and 156.25 ng/RNA/mL. The mRNA, and corresponding amino acid, sequence of the ABE8.8 (MA004) used in shown below in Table 18. Three days after transfection, genomic DNA was harvested from the hepatocytes, and assessed for base editing with next-generation sequencing of PCR amplicons generated around the target splice site. Several sites exhibited high editing efficiency. In particular, GA457 (GA458 is the cynomolgus equivalent), GA460, and GA461 showed high editing activity in both human and cynomolgus primary hepatocytes. See FIGS. 5A-5C, FIG. 6, and Tables 9-10.

TABLE 9
Editing activity in human primary hepatocytes

| | | Human hepatocytes- Editing % | | | | | |
|---|---|---|---|---|---|---|---|
| | | 2500, | 2500, | 1250, | 1250, | 625, | 625, |
| gRNA ID | Protospacer (5′-3′) | rep 1 | rep 2 | rep 1 | rep 2 | rep 1 | rep 2 |
| GA457 | GCCATCCTGCCAAGA | 33.96 | 31.31 | 26.49 | 24.86 | 17.24 | 15.39 |
| | ATGAG (SEQ ID NO: | | | | | | |
| | 467) | | | | | | |
| | GCAACTTACCCAGAG | | | | | | |
| GA459 | GCAAA (SEQ ID NO: | 8.5 | 8.69 | 5.46 | 6.15 | 3.79 | 3.89 |
| | 468) | | | | | | |
| | TATAGGAAAACCAGT | | | | | | |
| GA460 | GAGTC (SEQ ID NO: | 47.61 | 47.79 | 38.33 | 35.8 | 23.77 | 22.27 |
| | 469) | | | | | | |
| | TACTCACCTCTGCAT | | | | | | |
| GA461 | GCTCA (SEQ ID NO: | 40.42 | 39.43 | 32.82 | 32.38 | 21.81 | 22.31 |
| | 470) | | | | | | |

TABLE 10
Editing activity in cyno primary hepatocytes

| | | Cyno hepatocytes- Editing % | | | | | |
|---|---|---|---|---|---|---|---|
| gRNA ID | Protospacer (5′-3′) | 5000 | 2500 | 1250 | 625 | 312.5 | 156.25 |
| GA458 | GCCATCCTGCCAAGAACGAG | 35.91 | 27.25 | 22.74 | 16.07 | 10.65 | 6.06 |
| | (SEQ ID NO: 471) | | | | | | |
| GA460 | TATAGGAAAACCAGTGAGTC | 40.16 | 38.25 | 31.71 | 18.44 | 11.22 | 1.62 |
| | (SEQ ID NO: 469) | | | | | | |
| GA461 | TACTCACCTCTGCATGCTCA | 37.53 | 29.8 | 19.46 | 13.16 | 7.45 | 0.07 |
| | (SEQ ID NO: 470) | | | | | | |

Results presented in Tables 9 and 10 are to be understood to be representative of results that may be achieved in accordance with the teachings provided herein. Compositions for editing a TTR gene according to the invention may produce editing activity that varies from the activity set forth in Table 9 or Table 10 by 10% or more, 20% or more, 30% or more, 40% or more, 50% or more, 60% or more, 70% or more, 80% or more, 90% or more, or 100% or more. In some embodiments, the compositions provide editing activity that is within 100%, within 90%, within 80%, with 70%, within 60%, within 50%, within 40%, within 30% or more, within 20% or more, or within 10% of the activity as set forth in Table 9 or Table 10.

### Example 2: Off Target Analysis

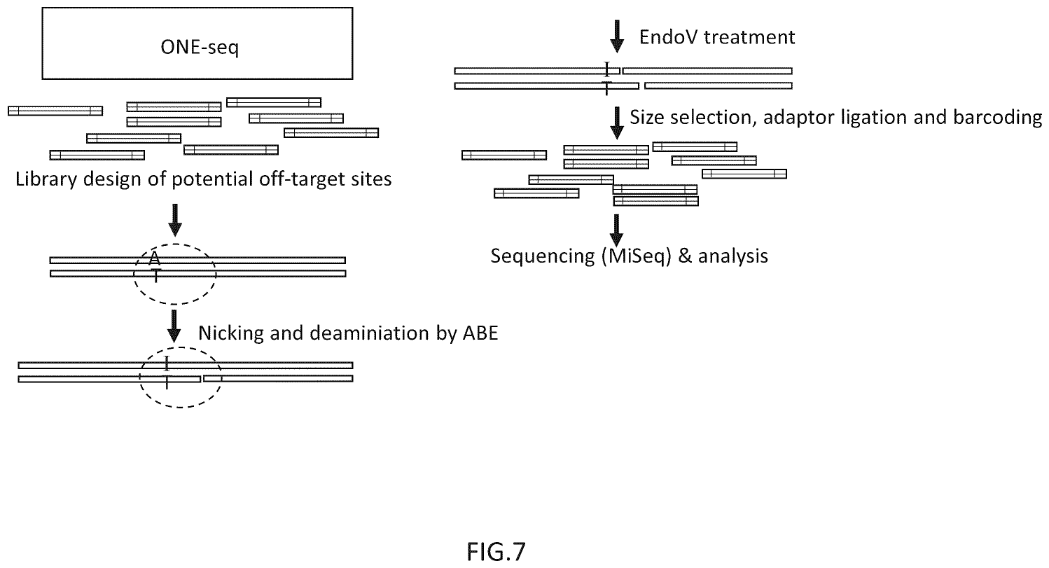

With a view towards establishing the safety of a base-editing therapy knocking down of TTR in the human liver in vivo, off-target mutagenesis analysis is assessed in primary human hepatocytes. Following the ONE-seq procedures detailed in PCT/US19/27788 (“Highly Sensitive in vitro Assays to Define Substrate Preferences and Sites of Nucleic-Acid Binding, Modifying, and Cleaving Agents”), off-target editing in human hepatocytes was assessed. A simplified flowchart of off-target analysis with the ONE-seq procedure is shown in FIG. 7. The in vitro biochemical assay ONE-seq was used to generate a list of candidate off-target sites and to determine the propensity of a ribonucleoprotein comprising the ABE8.8 base editor protein and each of the three protospacer guides sequences (GA457, GA460, and GA461) to cleave oligonucleotides in a library. The results from ONE-seq analysis of libraries generated for GA457, GA460, and GA461 are shown in Tables 15-17, with candidate off-target sites listed.

The methodology for ONE-seq is as follows: the design of a ONE-seq library starts with the computational identification of sites in a reference genome that have sequence homology to the on-target. For human ONE-seq libraries, the reference human genome (GRCh38, Ensembl v98, chromosomes ftp://ftp.ensembl.org/pub/release-98/fasta/homo_sapiens/dna/Homo_sapiens.GRCh38.dna.chromosome. {1-22,X,Y,MT}.fa and ftp://ftp.ensembl.org/pub/release-98/fasta/homo_sapiens/dna/Homo_sapiens.GRCh38.dna.nonchromosomal.fa), was searched for potential off-target sites with up to 6 mismatches to the protospacer sequence above, and sites with up to 4 mismatches plus up to 2 DNA or RNA bulges, using Cas-Designer v1.2 (rgenome.net/cas-designer/).

Sites with up to 6 mismatches and no bulges are referred to using a X<number of mismatches><number of bulges >code. As such, the on-target site is labelled as X⁰⁰; a site with 1 mismatch to the on-target and no bulges is labelled as X¹⁰, and so on. Sites with DNA bulges are referred to with a similar nomenclature, DNA<number of mismatches><number of bulges>. As such, a site with 4 mismatches to the on-target and 2 DNA bulges is labelled as DNA42. The same nomenclature is used for RNA bulges, but these are coded as RNA<number of mismatches><number of bulges>.

The protospacer sequences identified were extended by 10 nucleotides (nt) on both sides with adjacent sequence from the respective reference genome (these regions are herein referred to as the genomic context). These extended sequences were then padded by additional sequences up to a final length of approximately 200 nt, including 6 predefined constant regions of different nucleotide composition and sequence length; 2 copies of a 14-nt site-specific barcode, one on each side of the central protospacer sequence; and 2 distinct 11-nt unique molecular identifiers (UMIs), one on each side of the central protospacer sequence. The UMIs are used to correct for bias from PCR amplification, and the barcodes allow for the unambiguous identification of each site during analysis. The barcodes are selected from an initial list of 668,420 barcodes, which contain neither a CC nor a GG in their sequences, and each barcode has a Hamming distance of 2 from any other barcode. A custom Python script was used for designing the final library.

The final oligonucleotide libraries are synthesized by a commercial vendor (Agilent Technologies). Each library is PCR-amplified and subjected to 1.25x AMPure XP bead purification (Beckman Coulter). After incubation at 25° C. for 10 minutes in CutSmart buffer (New England Biolabs), RNP comprising 769 nM recombinant ABE8.8-m protein and 1.54 uM gRNA is mixed with 100 ng of the purified library and incubated at 37° C. for 8 hours. The RNP dose is derived from an analysis documenting that it is a super-saturating dose, ie, above the dose that achieves the maximum amount of on-target editing in the biochemical assay.

Proteinase K (New England Biolabs) is added to quench the reaction at 37° C. for 45 minutes, followed by 2x AMPure XP bead purification. The reaction is then serially incubated with EndoV (New England Biolabs) at 37° C. for 30 minutes, Klenow Fragment (New England Biolabs) at 37° C. for 30 minutes, and NEBNext Ultra II End Prep Enzyme Mix (New England Biolabs) at 20° C. for 30 minutes followed by 65° C. for 30 minutes, with 2x AMPure XP bead purification after each incubation. The reaction is ligated with an annealed adaptor oligonucleotide duplex at 20° C. for 1 hour to facilitate PCR amplification of the cleaved library products, followed by 2x AMPure XP bead purification. Size selection of the ligated reaction is performed on a PippinHT system (Sage Sciences) to isolate DNA of 150 to 200 bp on a 3% agarose gel cassette, followed by 2 rounds of PCR amplification to generate a barcoded library, which undergoes paired-end sequencing on an Illumina MiSeq System as described above.

Two cleavage products are obtained in a ONE-seq experiment. The PROTO side includes the part of the oligonucleotide upstream of the cleavage position, whereas the PAM side includes part of the oligonucleotide downstream of the cleavage position. In an ABE experiment, only the PROTO side is informative of editing activity (an A→G substitution); therefore, only this side is sequenced.

Paired-end reads were trimmed for sequencing adapters using trimmomatic v0.39 (Bolger et al., 2014) with custom Nextera adapters (PrefixPE/1: 5′-ACACTCTTTCCCTACACGACGCTCTTCCGATCT-3′ (SEQ ID NO: 1046); PrefixPE/2: 5′-GTGACTGGAGTTCAGACGTGTGCTCTTCCGATCT-3′ (SEQ ID NO: 1047); as specified in file) and parameters “ILLUMINACLIP: NEB custom.fa: 2:30:10:1: true LEADING: 0 TRAILING: 0 SLIDINGWINDOW: 4:30 MINLEN: 36”. For experiments with lower sequencing quality (VOL014), these parameters were set to “ILLUMINACLIP NEB custom.fa: 2:30:10:1: true LEADING: 2 TRAILING: 0 SLIDINGWINDOW: 30:30 MINLEN: 36”. Reads were then merged using FLASH v1.2.11 (Magoc and Salzberg, 2011) with parameters “--max-mismatch-density-0.25--max-overlap=160”. Merged reads were scanned for the constant sequences, barcodes and protospacer sequences unique to each site, and filtered to those with evidence of an A→G substitution in the editing window (defined as the 1-10 most PAM-distal positions of the protospacer). Duplicated reads were discarded.

For each site, the total number of edited reads was normalized to the total number of edited reads assigned to the on-target site, and this ratio defines the ONE-seq score for the site. Sites were ranked by ONE-seq score, and those with a score equal to or larger than 0.001, were selected for validation. A score equal to or larger than 0.001 encompasses sites that have down to 1000-fold less editing activity in the biochemical assay compared to editing of the on-target site. This threshold is based on the premise that in cells, if there is 100% on-target editing, 1/1000-fold less editing activity would translate to <0.1% off-target editing, which falls below the lower limit of detection of editing by NGS. Oligonucleotides with higher sequence counts reflect a higher propensity for Cas9/gRNA cleavage in vitro and hence for greater potential of off-target mutagenesis in cells.

Several candidate off-target sites were analyzed for off-target editing in human primary hepatocytes. Table 11 shows the results from validating 47 candidate off-target sites for guide RNA GA457, from cells co-transfected with gRNA and an equivalent amount of in vitro transcribed ABE8.8 mRNA (1:1 ratio by molecular weight) into primary human hepatocytes via MessengerMax reagent (Lipofectamine). The on-target site has high editing efficiency, while all off-target sites show little to no editing (less than 0.4% net editing).

TABLE 11
GA457 validation against 47 potential off-target candidate
sites in human primary hepatocytes

| | | SEQ | % Editing- human primary | | |
|---|---|---|---|---|---|
| GA457 | | ID | hepatocytes | | |
| ID | Sequence (5′-3′) | NO: | Treated | Untreated | Net |
| OT1 | GCCATCCTACCAGGAATGAA | 1048 | 0.28 | 0.13 | 0.15 |
| OT2 | GCCATCTTGCCAAGAAAAAG | 1049 | 0.12 | 0.09 | 0.03 |
| OT3 | GCCATACCTGCCATGAATGAG | 1050 | 0.2 | 0.19 | 0.01 |
| OT4 | GCCATCCTGACAGGAATGAG | 1051 | 0.24 | 0.38 | -0.14 |
| OT5 | ACCATCCTGCAAAGAATGAT | 1052 | 0.18 | 0.24 | -0.06 |
| OT6 | GCCATCCAATAAGAATGAG | 1053 | 0.69 | 0.32 | 0.37 |
| OT7 | GCCATCCTGACAAGTATGAG | 1054 | 0.29 | 0.22 | 0.07 |
| OT8 | CCATACCTGCCAAGAATGAA | 1055 | 0.18 | 0.15 | 0.03 |
| OT9 | TGCATCCTGCCAAAAATGGG | 1056 | 0.06 | 0.08 | -0.02 |
| OT10 | TGCATCCTGCCAAGAAGAAG | 1057 | 0.06 | 0.08 | -0.02 |
| OT11 | GCCATCCTCCAAGAATGCT | 1058 | 0.13 | 0.13 | 0 |
| OT12 | GCCATCTGCAAGAAGGAG | 1059 | 0.11 | 0.19 | -0.08 |
| OT13 | GCCATCCTATCAAGAATAAA | 1060 | 0.22 | 0.2 | 0.02 |
| OT14 | TCCATCCTGTAAGAATGAG | 1061 | 0.03 | 0.05 | -0.02 |
| OT15 | GGCCATCTGCCAAGAAGGAT | 1062 | 0.12 | 0.13 | -0.01 |
| OT16 | ACCATCCTGCCAGCAATGTG | 1063 | 0.17 | 0.12 | 0.05 |
| OT17 | TCCATCCTACTAAGAATGAG | 1064 | 0.11 | 0.13 | -0.02 |
| OT18 | GGTATCCTGCCAAGAATGGA | 1065 | 0.09 | 0.1 | -0.01 |
| OT19 | TCCATCCTGCCAAGAATTGC | 1066 | 0.14 | 0.07 | 0.07 |
| OT20 | GCCATCTGCAAGAATGAG | 1067 | 0.15 | 0.18 | -0.03 |
| OT21 | GCCATCCTGCAAATATGAG | 1068 | 0.04 | 0.07 | -0.03 |
| OT22 | ACCATCCTGTCAAGAATCAA | 1069 | 0.2 | 0.15 | 0.05 |
| OT23 | GCCATACTAACAAGAATGAG | 1070 | 0.25 | 0.23 | 0.02 |
| OT24 | GTGATCCTGCCAGGAATAAG | 1071 | 0.08 | 0.11 | -0.03 |
| OT25 | GCCATCAAGCAAGAATGAG | 1072 | 0.3 | 0.27 | 0.03 |
| OT26 | GCCATCCTCACAAGTATGAG | 1073 | 0.19 | 0.22 | -0.03 |
| OT27 | ACCATCCAGCAAGAATGAG | 1074 | 0.43 | 0.32 | 0.11 |
| OT28 | GCCATATGCCAAAAAGGAG | 1075 | 0.24 | 0.24 | 0 |
| OT29 | GACATCCTGTCAAGGATCAG | 1076 | 0.21 | 0.16 | 0.05 |
| OT30 | GCCATAGCCAAAAATGAA | 1077 | 0.18 | 0.27 | -0.09 |
| OT31 | GCCATAAGCCAAAGAATGAC | 1078 | 0.09 | 0 | 0.09 |
| OT32 | GCCATCCTAACAAGTATGAG | 1079 | 0.25 | 0.45 | -0.2 |
| OT33 | CATATCCTGCCAGAATGAG | 1080 | 0.15 | 0.21 | -0.06 |
| OT34 | TACATCCTACCAAGGAATCAG | 1081 | 0.29 | 0.26 | 0.03 |
| OT35 | CCCATCCTGCCAAGAAGTGT | 1082 | 0.08 | 0.06 | 0.02 |
| OT36 | GCCATCCTACAAAAATGAG | 1083 | 0.16 | 0.21 | -0.05 |
| OT37 | GTCATCCTGCCAGGAATGAA | 1084 | 0.09 | 0.07 | 0.02 |
| OT38 | GCCATATCTGCCAAGAATGCG | 1085 | 0.16 | 0.16 | 0 |
| OT39 | TCCATCCTGTCAAGAATGTG | 1086 | 0.05 | 0.04 | 0.01 |
| OT40 | TCCATCCTCCAGAATGAG | 1087 | 0.07 | 0.08 | -0.01 |
| OT41 | GCCATGCTGCCAAGAATGAT | 1088 | 0.15 | 0.16 | -0.01 |
| OT42 | GCTATCCTGCCAGAATGAG | 1089 | 0.07 | 0.07 | 0 |
| OT43 | TGCATCCTGACAAGAAATAG | 1090 | 0.34 | 0.24 | 0.1 |
| OT44 | TCCATAGCCAAGAATGAG | 1091 | 0.43 | 0.27 | 0.16 |
| OT45 | ACCATCTGTCAAGAATGAG | 1092 | 0.26 | 0.21 | 0.05 |
| OT46 | GCCATCCCGCCAGGAATTAT | 1093 | 0.08 | 0.07 | 0.01 |
| OT47 | ACCATCCTTCCAAGAAGATG | 1094 | 0.14 | 0.12 | 0.02 |

GA459, GA460, and GA461 were similarly also assessed for off-target editing as shown in Tables 12, 13, and 14, respectively. While the on-target site, for each guide, shows high editing efficiency in the treated groups compared to the control groups, there is little to no off-target editing observed at candidate off-target sites.

TABLE 12
GA459 validation against 6 potential off-target candidate sites in human
primary hepatocytes

| | | SUM Editing %- Human Primary Hepatocytes | | | | |
|---|---|---|---|---|---|---|
| GA459 | | Treat | Treat | Untreated | Untreated | Untreated |
| ID | Sequence (5′-3′) | rep 1 | rep 2 | rep 1 | rep 2 | rep 3 |
| On- | GCAACTTACCCAGAGGCAA | 14.17 | 14.29 | 0.67 | 0.57 | 0.73 |
| target | A (SEQ ID NO: 468) | | | | | |
| OT1 | ACAAATTACCCAGAGGAAA | 1.19 | 1.25 | 1.31 | 1.28 | 1.27 |
| | A (SEQ ID NO: 1095) | | | | | |
| OT3 | TCAACTTACCCAGAGTCAA | 0.98 | 0.82 | 0.63 | 0.68 | 0.8 |
| | A (SEQ ID NO: 1096) | | | | | |
| OT4 | GCAACTTGCCCAGAGGCAC | 0.92 | 1.07 | 0.87 | 0.99 | 0.79 |
| | A (SEQ ID NO: 1097) | | | | | |
| OT5 | GCAACATACCCAGTGGCAA | 1.07 | 0.91 | 0.87 | 0.86 | 0.92 |
| | A (SEQ ID NO: 1098) | | | | | |
| OT6 | GCAGCCTACCCAGAGGCAA | 1.02 | 1.1 | 0.97 | 1 | 1.05 |
| | A (SEQ ID NO: 1099) | | | | | |
| OT7 | GCAACTCCCCCAGAGGCAA | 1.45 | 1.4 | 1.25 | 1.13 | 1.37 |
| | A (SEQ ID NO: 1100) | | | | | |

TABLE 13
GA460 validation against 3 potential off-target candidate sites in human
primary hepatocytes

| | | SUM Editing %- Human Primary Hepatocytes | | | | |
|---|---|---|---|---|---|---|
| GA460 | | Treat | Treat | Untreated | Untreated | Untreated |
| ID | Sequence (5′-3′) | rep 1 | rep 2 | rep 1 | rep 2 | rep 3 |
| On- | TATAGGAAAACCAGTGAGT | 76.4 | 74.3 | 1.54 | 1.17 | 1.41 |
| target | C (SEQ ID NO: 469) | | | | | |
| OT1 | TAGAGGAAAACCAGTCAGT | 1.44 | 1.6 | 1.51 | 1.35 | 1.61 |
| | C (SEQ ID NO: 1100) | | | | | |
| OT2 | CATAGGAAAACCAGTGAGT | 5.67 | 6.6 | 1.18 | 0.98 | 1.24 |
| | T (SEQ ID NO: 1101) | | | | | |
| OT3 | TAAAGGAAAACCAGTGGGT | 1.26 | 1.29 | 1.31 | 1.02 | 1.53 |
| | C (SEQ ID NO: 1102) | | | | | |

TABLE 14
GA461 validation against 4 potential off-target candidate sites in human
primary hepatocytes

| | | SUM Editing %- Human Primary Hepatocytes | | | | |
|---|---|---|---|---|---|---|
| GA461 | | Treat | Treat | Untreated | Untreated | Untreated |
| ID | Sequence (5′-3′) | rep 1 | rep 2 | rep 1 | rep 2 | rep 3 |
| On- | TACTCACCTCTGCATGCTC | ND | 58.9 | 0.39 | 0.37 | 0.35 |
| target | A (SEQ ID NO: 470) | | | | | |
| OT1 | TACACAACTGTGCATGCTC | 0.93 | 0.95 | 0.89 | 0.86 | 0.82 |
| | A (SEQ ID NO: 1104) | | | | | |
| OT2 | TATTCACCTCTGCATGCTC | 0.17 | 0.18 | 0.23 | 0.16 | 0.2 |
| | T (SEQ ID NO: 1105) | | | | | |
| OT3 | TACTTACCTCTGCTTGCTC | 0.28 | 0.35 | 0.28 | 0.23 | 0.34 |
| | A (SEQ ID NO: 1106) | | | | | |
| OT4 | TACACACCTCTACATGCTC | 0.67 | 0.94 | 0.75 | 0.62 | ND |
| | A (SEQ ID NO: 1107) | | | | | |

Table 15 provides some results for off-target editing with the GA457 guide.

Table 16 provides some results for off-target editing with the GA460 guide.

Table 17 provides some results for off-target editing with the GA461 guide.

Results presented in Tables 11, 13, 14, 15, 16, and 17 are to be understood to be representative of results that may be achieved in accordance with the teachings provided herein. Compositions for editing a TTR gene according to the invention may produce total off-target editing activity that varies from the activity set forth in Table 11, 13, 14, 15, 16, or 19 or discussed regarding GA457, 460, or 461. For example, the compositions may produce total off-target editing activity that varies from the activity set forth in Table 11, 13, 14, 15, 16, or 17 or discussed regarding GA457, 460, or 461 by 10% or more, 20% or more, 30% or more, 40% or more, 50% or more, 60% or more, 70% or more, 80% or more, 90% or more, or 100% or more, for one or more off target site set forth in Table 11, 13, 14, 15, 16, or 17 or discussed regarding GA457, 460, or 461. In some embodiments, the compositions provide total off-target editing activity that is within 100%, within 90%, within 80%, with 70%, within 60%, within 50%, within 40%, within 30% or more, within 20% or more, or within 10% of the activity as set forth in Table 11, 13, 14, 15, 16, or 17 or discussed regarding GA457, 460, or 461 for one or more site set forth in Table 11, 13, 14, 15, 16, or 17 or discussed regarding GA457, 460, or 461. In some embodiments, the compositions produce off-target editing activity that is less than or equal to the activity set forth in Table 11, 13, 14, 15, 16, or 17 or discussed regarding GA457, 460, or 461 for one or more site set forth in Table 11, 13, 14, 15, 16, or 17 or discussed regarding GA457, 460, or 461. In some embodiments, the compositions produce no off-target editing activity for one or more site set forth in Table 11, 13, 14, 15, 16, or 17 or discussed regarding GA457, 460, or 461.

TABLE 15
Some GA457 Candidate Off-target Sites

| | | | SEQ | |
|---|---|---|---|---|
| | | | ID | |
| Chromosome | Location | Sequence (5′-3′) | NO: | Alignment |
| 1 | 214915487 | TGCATCCTGCCAAAAATGGGGAG | 1108 | X50 |
| 2 | 162088240 | GCCATCTTGCCAAGAAAAAGGGG | 1109 | X30 |
| 3 | 143033429 | AACATCCTGCAAGAATAAGAAG | 1110 | RNA41 |
| 4 | 148246005 | GCCATCCAATAAGAATGAGTGG | 1111 | RNA31 |
| 5 | 171975758 | GTCATCCTGCCAAAATAAGGAGG | 1112 | X60 |
| 6 | 11558428 | CCCATACAGCCAAGAATGAGAAA | 1113 | X50 |
| 7 | 136711066 | TCCATCCTACTAAGAATGAGGAG | 1114 | X40 |
| 8 | 64649745 | ACCATCCTGACAAGTGTGAGGCA | 1115 | X60 |
| 9 | 13461786 | GTGATCCTGCCAGGAATAAGGAG | 1116 | X50 |
| 10 | 84701101 | GCCACCCTCCAAGGATCTGAGG | 1117 | RNA41 |
| 11 | 36962784 | GCCATACTAACAAGAATGAGGTG | 1118 | X40 |
| 12 | 18423002 | GCCATCCTCAAGAATGGGAAA | 1119 | RNA32 |
| 13 | 109466404 | ACCATCCTGTCAAGAATCAAGAG | 1120 | X50 |
| 14 | 43842189 | GCCAACCTGACAAATGTGAGG | 1121 | RNA32 |
| 15 | 38417527 | ACCATCCTTCCAAGAAGATGGGG | 1122 | X50 |
| 16 | 19426632 | GCCATCCAGCCAAGCAAGAAGGG | 1123 | X40 |
| 17 | 42774058 | GTCATCACTGCCAAGAACAAGAGG | 1124 | DNA31 |
| 18 | 6852692 | GCCATCCTGTAAGAATAAGGAT | 1125 | RNA41 |
| 19 | 55221349 | ACCATCCTGCCAGCAATGTGAGG | 1126 | X40 |
| 20 | 56309168 | TCCATCCTGAAGAATGAATAG | 1127 | RNA32 |
| 21 | 40793316 | GCCATATCTGCCAAGAATGCGGAG | 1128 | DNA31 |
| 22 | 34535097 | GATATCCTCACAAGAATGAGTGA | 1129 | X50 |
| X | 54383781 | GCTCTACTGCCAAGAAAGTGG | 1130 | RNA32 |
| Y | 6961334 | GCCATCCACCAAGAAAGCGGAG | 1131 | RNA41 |

Additional examples of GA457 off-target sites are presented in U.S. Provisional Patent Application No. 63/322,182, filed Mar. 21, 2022.

TABLE 16
Some GA460 Candidate Off-target Sites

| | | | SEQ ID | |
|---|---|---|---|---|
| Chromosome | Location | Sequence | NO: | Alignment |
| 1 | 114689517 | TATAAGAAAACCAGTGTCTCTGG | 1132 | X30 |
| 2 | 165173430 | TATAGTGAACCAGTGAGGCAGG | 1133 | RNA31 |
| 3 | 56975861 | TGTAGAAAAACCAGTGAATAAGA | 1134 | X50 |
| 4 | 175627437 | GATAGGAAAACCATGAGGGGGT | 1135 | RNA41 |
| 5 | 115033087 | TATAGGAAACCAATGAGTGCTG | 1136 | RNA31 |
| 6 | 132497708 | TATAGGTAAACCAGAGTAGGC | 1137 | RNA32 |
| 7 | 40539970 | AGTAGGAAAACCAGTATATAGGG | 1138 | X60 |
| 8 | 77342779 | AATAGGAAAACCATTTTCAGG | 1139 | RNA32 |
| 9 | 73825991 | AATAGGAAAACCAGTAAAATAGG | 1140 | X50 |
| 10 | 112702031 | CATACGAAGTCCATGAGTCAGG | 1141 | RNA41 |
| 11 | 60264073 | ATATGGAAAACCAGAGAATCAGG | 1142 | X60 |
| 12 | 103497054 | GATAGAAACACCATGAATCAGG | 1143 | RNA41 |
| 13 | 31477698 | TATATGAAACCAGTAAGTTTGG | 1144 | RNA31 |
| 14 | 91758013 | TATAGGTGTAAACCAGTGTGCCTAG | 1145 | DNA42 |
| 15 | 28238271 | TATAGGAGAAACAGTGAATAGGA | 1146 | X50 |
| 16 | 74819495 | AAAATGTAAACCAGTAAGCCCGG | 1147 | X60 |
| 17 | 44943044 | TGTAGGAAGAACCAGTGGATCGGG | 1148 | DNA31 |
| 18 | 1915908 | AAAAGGAAAGCCAGTGACCTGG | 1149 | RNA41 |
| 19 | 44246762 | AAATAGAAAACCAGTAAGTCATG | 1150 | X60 |
| 20 | 24558049 | TATAGGAAAACAGGAACTCTGG | 1151 | RNA31 |
| 21 | 13185396 | TATAATAAAACCAGTGATAAGGG | 1152 | X50 |
| 22 | 38987774 | TGTAGGAAAACATATGATCAGG | 1153 | RNA41 |
| X | 134161037 | AATAGGATAACCAGTCAGTAGGG | 1154 | X40 |
| Y | 15254846 | TATAACATAACCAATAGGTCAGG | 1155 | X60 |

Additional examples of GA460 off-target sites are presented in U.S. Provisional Patent Application No. 63/322,182, filed Mar. 21, 2022.

TABLE 17
Some GA461 Candidate Off-target sites

| | | | SEQ | |
|---|---|---|---|---|
| | | | ID | |
| Chromosome | Location | Sequence (5′-3′) | NO: | Alignment |
| 1 | 110474222 | TACTCACCTCTACATGCTCAGTG | 1156 | X20 |
| 2 | 136311439 | TACAGCACCTCTGCATTGCCAGGG | 1157 | DNA41 |
| 3 | 159697502 | TATTCACCTCTGCATCTCCAGGG | 1158 | X40 |
| 4 | 3846305 | CTCTCACCTCTGCATGACAAGC | 1159 | RNA41 |
| 5 | 174698295 | TACTCACAATGCATGCTAAAGG | 1160 | RNA31 |
| 6 | 31933576 | TACTCACCTCTGCCTTCCTTTGT | 1161 | X60 |
| 7 | 53373361 | TTATCACCCTGCATGTTCAGGG | 1162 | RNA31 |
| 8 | 77161406 | TACTGACCTTTCCATCTCACTG | 1163 | RNA41 |
| 9 | 101247864 | TACTCACTCAGCATGTTCAGAG | 1164 | RNA31 |
| 10 | 4684859 | GAATAACCTCTGATGGTCAAGG | 1165 | RNA41 |
| 11 | 85210644 | TATTCACCTCTGCATGCTCTGAG | 1166 | X30 |
| 12 | 117261587 | TATTCAGCAATGCATGTCAAGG | 1167 | RNA41 |
| 13 | 94452599 | TACTTACCTTACATGTTCAAGG | 1168 | RNA31 |
| 14 | 64054638 | TACTCAACTCTGCTGCTATAGC | 1169 | RNA41 |
| 15 | 95108931 | TACTCAACTCTGCTGCTCTAGG | 1170 | RNA21 |
| 16 | 56094894 | TACTAACCTTGCCAGCTGAGGG | 1171 | RNA41 |
| 17 | 71161191 | TGCTCACCCCACATGCTCATGG | 1172 | RNA31 |
| 18 | 3284286 | TTTTCTACTCTGCATAATCATGG | 1173 | X60 |
| 19 | 45241812 | ACCTCACCTCTGCCTGCTCTGGG | 1174 | X40 |
| 20 | 13122061 | TACTCAACTGCATTCTCAGGG | 1175 | RNA22 |
| 21 | 45735600 | CACTCACACTACATGCTCTTGG | 1176 | RNA41 |
| 22 | 20392740 | TCCACACCTCTCGGCAAGCTGAGGG | 1177 | DNA42 |
| X | 39842774 | TATATACCTCTGCATGTTCAGAG | 1178 | X50 |
| Y | 20738820 | TAGACACATAAGCATGCTCACAG | 1179 | X60 |

Additional examples of GA461 off-target sites are presented in U.S. Provisional Patent Application No. 63/322,182, filed Mar. 21, 2022.

TABLE 18
ABE variant sequences
MA004 mRNA and protein sequences

| Region | Sequence | |
|---|---|---|
| Full | mRNA | Au′GAGCGAGGu′GGAGu′u′CAGCCACGAGu′ACu′GGAu′GCGGCAC |
| sequence | | GCCCu′GACCCu′GGCCAAGCGGGCCCGGGACGAGCGGGAGGu′GCCCG |
| | | u′GGGCGCCGu′GCu′GGu′GCu′GAACAACCGGGu′GAu′CGGCGAGG |
| | | GCu′GGAACCGGGCCAu′CGGCCu′GCACGACCCCACCGCCCACGCCGA |
| | | GAu′CAu′GGCCCu′GCGGCAGGGGGGCCu′GGu′GAu′GCAGAACu′A |
| | | CCGGCu′GAu′CGACGCCACCCu′Gu′ACGu′GACCu′u′CGAGCCCu′ |
| | | GCGu′GAu′Gu′GCGCCGGCGCCAu′GAu′CCACAGCCGGAu′CGGCCG |
| | | GGu′GGu′Gu′u′CGGCGu′GCGGAACGCCAAGACCGGCGCCGCCGGCA |
| | | GCCu′GAu′GGACGu′GCu′GCACCACCCCGGCAu′GAACCACCGGGu′ |
| | | GGAGAu′CACCGAGGGCAu′CCu′GGCCGACGAGu′GCGCCGCCCu′GC |
| | | u′Gu′GCCGGu′u′Cu′u′CCGGAu′GCCCCGGCGGGu′Gu′u′CAACG |
| | | CCCAGAAGAAGGCCCAGAGCAGCACCGACAGGAAAu′AAGAGAGAAAAG |
| | | AAGAGu′AAGAAGAAAu′Au′AAGAGCCACCAGCGGCGGCAGCAGCGGC |
| | | GGCAGCAGCGGCAGCGAGACACCCGGCACCAGCGAGAGCGCCACCCCCG |
| | | AGAGCAGCGGCGGCAGCAGCGGCGGCAGCGACAAGAAGu′ACAGCAu′C |
| | | GGCCu′GGCCAu′CGGCACCAACAGCGu′GGGCu′GGGCCGu′GAu′CA |
| | | CCGACGAGu′ACAAGGu′GCCCAGCAAGAAGu′u′CAAGGu′GCu′GGG |
| | | CAACACCGACCGGCACAGCAu′CAAGAAGAACCu′GAu′CGGCGCCCu′ |
| | | GCu′Gu′u′CGACAGCGGCGAGACAGCCGAGGCCACCCGGCu′GAAGCG |
| | | GACCGCCCGGCGGCGGu′ACACCCGGCGGAAGAACCGGAu′Cu′GCu′A |
| | | CCu′GCAGGAGAu′Cu′u′CAGCAACGAGAu′GGCCAAGGu′GGACGAC |
| | | AGCu′u′Cu′u′CCACCGGCu′GGAGGAGAGCu′u′CCu′GGu′GGAGG |
| | | AGGACAAGAAGCACGAGCGGCACCCCAu′Cu′u′CGGCAACAu′CGu′G |
| | | GACGAGGu′GGCCu′ACCACGAGAAGu′ACCCCACCAu′Cu′ACCACCu |
| | | ′GCGGAAGAAGCu′GGu′GGACAGCACCGACAAGGCCGACCu′GCGGCu |
| | | ′GAu′Cu′ACCu′GGCCCu′GGCCCACAu′GAu′CAAGu′u′CCGGGGC |
| | | CACu′u′CCu′GAu′CGAGGGCGACCu′GAACCCCGACAACAGCGACGu |
| | | ′GGACAAGCu′Gu′u′CAu′CCAGCu′GGu′GCAGACCu′ACAACCAGC |
| | | u′Gu′u′CGAGGAGAACCCCAu′CAACGCCAGCGGCGu′GGACGCCAAG |
| | | GCCAu′CCu′GAGCGCCCGGCu′GAGCAAGAGCCGGCGGCu′GGAGAAC |
| | | Cu′GAu′CGCCCAGCu′GCCCGGCGAGAAGAAGAACGGCCu′Gu′u′CG |
| | | GCAACCu′GAu′CGCCCu′GAGCCu′GGGCCu′GACCCCCAACu′u′CA |
| | | AGAGCAACu′u′CGACCu′GGCCGAGGACGCCAAGCu′GCAGCu′GAGC |
| | | AAGGACACCu′ACGACGACGACCu′GGACAACCu′GCu′GGCCCAGAu′ |
| | | CGGCGACCAGu′ACGCCGACCu′Gu′u′CCu′GGCCGCCAAGAACCUu′G |
| | | AGCGACGCCAu′CCu′GCu′GAGCGACAu′CCu′GCGGGu′GAACACCG |
| | | AGAu′CACCAAGGCCCCCCu′GAGCGCCAGCAu′GAu′CAAGCGGu′AC |
| | | GACGAGCACCACCAGGACCu′GACCCu′GCu′GAAGGCCCu′GGu′GCG |
| | | GCAGCAGCu′GCCCGAGAAGu′ACAAGGAGAu′Cu′u′Cu′u′CGACCA |
| | | GAGCAAGAACGGCu′ACGCCGGCu′ACAu′CGACGGCGGCGCCAGCCAG |
| | | GAGGAGu′u′Cu′ACAAGu′u′CAu′CAAGCCCAu′CCu′GGAGAAGAu |
| | | ′GGACGGCACCGAGGAGCu′GCu′GGu′GAAGCu′GAACCGGGAGGACC |
| | | u′GCu′GCGGAAGCAGCGGACCUu′u′CGACAACGGCAGCAu′CCCCCAC |
| | | CAGAu′CCACCu′GGGCGAGCu′GCACGCCAu′CCu′GCGGCGGCAGGA |
| | | GGACu′u′Cu′ACCCCu′u′CCu′GAAGGACAACCGGGAGAAGAu′CGA |
| | | GAAGAu′CCu′GACCu′u′CCGGAu′CCCCu′ACu′ACGu′GGGCCCCC |
| | | u′GGCCCGGGGCAACAGCCGGu′u′CGCCu′GGAu′GACCCGCAAGAGC |
| | | GAGGAGACAAu′CACCCCCu′GGAACu′u′CGAGGAGGu′GGu′GGACA |
| | | AGGGCGCCAGCGCCCAGAGCu′u′CAu′CGAGCGGAu′GACCAACu′u′ |
| | | CGACAAGAACCu′GCCCAACGAGAAGGu′GCu′GCCCAAGCACAGCCu′ |
| | | GCu′Gu′ACGAGu′ACu′u′CACCGu′Gu′ACAACGAGCu′GACCAAGG |
| | | u′GAAGu′ACGu′GACCGAGGGCAu′GCGGAAGCCCGCCu′u′CCu′GA |
| | | GCGGCGAGCAGAAGAAGGCCAu′CGu′GGACCu′GCu′Gu′u′CAAGAC |
| | | CAACCGGAAGGu′GACCGu′GAAGCAGCu′GAAGGAGGACu′ACu′u′C |
| | | AAGAAGAu′CGAGu′GCu′u′CGACAGCGu′GGAGAu′CAGCGGCGu′G |
| | | GAGGACCGGu′u′CAACGCCAGCCu′GGGCACCu′ACCACGACCu′GCu |
| | | ′GAAGAu′CAu′CAAGGACAAGGACu′u′CCu′GGACAACGAGGAGAAC |
| | | GAGGACAu′CCu′GGAGGACAu′CGu′GCu′GACCCu′GACCCu′Gu′u |
| | | ′CGAGGACCGGGAGAu′GAu′CGAGGAGCGGCu′GAAGACCUu′ACGCCC |
| | | ACCu′Gu′u′CGACGACAAGGu′GAu′GAAGCAGCu′GAAGCGGCGGCG |
| | | Gu′ACACCGGCu′GGGGCCGGCu′GAGCCGGAAGCu′GAu′CAACGGCA |
| | | u′CCGGGACAAGCAGAGCGGCAAGACCAu′CCu′GGACu′u′CCu′CAA |
| | | GAGCGACGGCu′u′CGCCAACCGGAACu′u′CAu′GCAGCu′GAu′CCA |
| | | CGACGACAGCCu′GACCu′u′CAAGGAGGACAu′CCAGAAGGCCCAGGu |
| | | ′GAGCGGCCAGGGCGACAGCCu′GCACGAGCACAu′CGCCAACCu′GGC |
| | | CGGCAGCCCCGCCAu′CAAGAAGGGCAu′CCu′GCAGACCGu′GAAGGu |
| | | ′GGu′GGACGAGCu′GGu′GAAGGu′GAu′GGGCCGGCACAAGCCCGAG |
| | | AACAu′CGu′GAu′CGAGAu′GGCCCGGGAGAACCAGACCACCCAGAAG |
| | | GGCCAGAAGAACAGCCGGGAGCGGAu′GAAGCGGAu′CGAGGAGGGCAu |
| | | ′CAAGGAGCu′GGGCAGCCAGAu′CCu′GAAGGAGCACCCCGu′GGAGA |
| | | ACACCCAGCu′GCAGAACGAGAAGCu′Gu′ACCu′Gu′ACu′ACCu′GC |
| | | AGAACGGCCGGGACAu′Gu′ACGu′GGACCAGGAGCu′GGACAu′CAAC |
| | | CGGCu′GAGCGACu′ACGACGu′GGACCACAu′CGu′GCCCCAGAGCu′ |
| | | u′CCu′GAAGGACGACAGCAu′CGACAACAAGGu′GCu′GACCCGGAGC |
| | | GACAAGAACCGGGGCAAGAGCGACAACGu′GCCCAGCGAGGAGGu′GGu |
| | | ′GAAGAAGAu′GAAGAACu′ACu′GGCGGCAGCu′GCu′GAACGCCAAG |
| | | Cu′GAu′CACCCAGCGGAAGu′u′CGACAACCu′GACCAAGGCCGAGCG |
| | | GGGCGGCCu′GAGCGAGCu′GGACAAGGCCGGCu′u′CAu′CAAGCGGC |
| | | AGCu′GGu′GGAGACACGGCAGAu′CACCAAGCACGu′GGCCCAGAu′C |
| | | Cu′GGACAGCCGGAu′GAACACCAAGu′ACGACGAGAACGACAAGCu′G |
| | | Au′CCGGGAGGu′GAAGGu′GAu′CACCCu′CAAGAGCAAGCu′GGu′G |
| | | AGCGACu′u′CCGGAAGGACu′u′CCAGu′u′Cu′ACAAGGu′GCGGGA |
| | | GAu′CAACAACu′ACCACCACGCCCACGACGCCu′ACCu′GAACGCCGu |
| | | ′GGu′GGGCACCGCCCu′GAu′CAAGAAGu′ACCCCAAGCu′GGAGAGC |
| | | GAGu′u′CGu′Gu′ACGGCGACu′ACAAGGu′Gu′ACGACGu′GCGGAA |
| | | GAu′GAu′CGCCAAGAGCGAGCAGGAGAu′CGGCAAGGCCACCGCCAAG |
| | | u′ACu′u′Cu′u′Cu′ACAGCAACAu′CAu′GAACu′u′Cu′u′CAAGA |
| | | CCGAGAu′CACCCu′GGCCAACGGCGAGAu′CCGGAAGCGGCCCCu′GA |
| | | u′CGAGACAAACGGCGAGACAGGCGAGAu′CGu′Gu′GGGACAAGGGCC |
| | | GGGACu′u′CGCCACCGu′GCGGAAGGu′GCu′GAGCAu′GCCCCAGGu |
| | | ′GAACAu′CGu′GAAGAAGACCGAGGu′GCAGACCGGCGGCu′u′CAGC |
| | | AAGGAGAGCAu′CCu′GCCCAAGCGGAACAGCGACAAGCu′GAu′CGCC |
| | | CGGAAGAAGGACu′GGGACCCCAAGAAGu′ACGGCGGCu′u′CGACAGC |
| | | CCCACCGu′GGCCu′ACAGCGu′GCu′GGu′GGu′GGCCAAGGu′GGAG |
| | | AAGGGCAAGAGCAAGAAGCu′CAAGAGCGu′GAAGGAGCu′GCu′GGGC |
| | | Au′CACCAu′CAu′GGAGCGGAGCAGCu′u′CGAGAAGAACCCCAu′CG |
| | | ACu′u′CCu′GGAGGCCAAGGGCu′ACAAGGAGGu′GAAGAAGGACCU′ |
| | | GAu′CAu′CAAGCu′GCCCAAGu′ACAGCCu′Gu′u′CGAGCu′GGAGA |
| | | ACGGCCGGAAGCGGAu′GCu′GGCCAGCGCCGGCGAGCu′GCAGAAGGG |
| | | CAACGAGCu′GGCCCu′GCCCAGCAAGu′ACGu′GAACu′u′CCu′Gu′ |
| | | ACCu′GGCCAGCCACu′ACGAGAAGCu′GAAGGGCAGCCCCGAGGACAA |
| | | CGAGCAGAAGCAGCu′Gu′u′CGu′GGAGCAGCACAAGCACu′ACCu′G |
| | | GACGAGAu′CAu′CGAGCAGAu′CAGCGAGu′u′CAGCAAGCGGGu′GA |
| | | u′CCu′GGCCGACGCCAACCu′GGACAAGGu′GCu′GAGCGCCu′ACAA |
| | | CAAGCACCGGGACAAGCCCAu′CCGGGAGCAGGCCGAGAACAu′CAu′C |
| | | CACCu′Gu′u′CACCCu′GACCAACCu′GGGCGCCCCCGCCGCCu′u′C |
| | | AAGu′ACu′u′CGACACCACCAu′CGACCGGAAGCGGu′ACACCAGCAC |
| | | CAAGGAGGu′GCu′GGACGCCACCCu′GAu′CCACCAGAGCAu′CACCG |
| | | GCCu′Gu′ACGAGACACGGAu′CGACCu′GAGCCAGCu′GGGCGGCGAC |
| | | GAGGGCGCCGACAAGCGGACCGCCGACGGCAGCGAGu′u′CGAGAGCCC |
| | | CAAGAAGAAGCGGAAGGu′Gu′GAGCGGCCGCu′u′AAu′u′AAGCu′G |
| | | CCu′u′Cu′GCGGGGCu′u′GCCu′u′Cu′GGCCAu′GCCCu′u′Cu′u |
| | | ′Cu′Cu′CCCu′u′GCACCu′Gu′ACCu′Cu′u′GGu′Cu′u′u′GAAu |
| | | ′AAAGCCu′GAGu′AGGAAGu′Cu′AGA (SEQ ID NO: 1180) |
| | protein | MSEVEFSHEYWMRHALTLAKRARDEREVPVGAVLVLNNRVIGEGWNRAI |
| | | GLHDPTAHAEIMALRQGGLVMQNYRLIDATLYVTFEPCVMCAGAMIHSR |
| | | IGRVVFGVRNAKTGAAGSLMDVLHHPGMNHRVEITEGILADECAALLCR |
| | | FFRMPRRVFNAQKKAQSSTDSGGSSGGSSGSETPGTSESATPESSGGSS |
| | | GGSDKKYSIGLAIGTNSVGWAVITDEYKVPSKKFKVLGNTDRHSIKKNL |
| | | IGALLFDSGETAEATRLKRTARRRYTRRKNRICYLQEIFSNEMAKVDDS |
| | | FFHRLEESFLVEEDKKHERHPIFGNIVDEVAYHEKYPTIYHLRKKLVDS |
| | | TDKADLRLIYLALAHMIKFRGHFLIEGDLNPDNSDVDKLFIQLVQTYNQ |
| | | LFEENPINASGVDAKAILSARLSKSRRLENLIAQLPGEKKNGLFGNLIA |
| | | LSLGLTPNFKSNFDLAEDAKLQLSKDTYDDDLDNLLAQIGDQYADLFLA |
| | | AKNLSDAILLSDILRVNTEITKAPLSASMIKRYDEHHQDLTLLKALVRO |
| | | QLPEKYKEIFFDQSKNGYAGYIDGGASQEEFYKFIKPILEKMDGTEELL |
| | | VKLNREDLLRKQRTFDNGSIPHQIHLGELHAILRRQEDFYPFLKDNREK |
| | | IEKILTFRIPYYVGPLARGNSRFAWMTRKSEETITPWNFEEVVDKGASA |
| | | QSFIERMTNFDKNLPNEKVLPKHSLLYEYFTVYNELTKVKYVTEGMRKP |
| | | AFLSGEQKKAIVDLLFKTNRKVTVKQLKEDYFKKIECFDSVEISGVEDR |
| | | FNASLGTYHDLLKIIKDKDFLDNEENEDILEDIVLTLTLFEDREMIEER |
| | | LKTYAHLFDDKVMKQLKRRRYTGWGRLSRKLINGIRDKQSGKTILDFLK |
| | | SDGFANRNFMQLIHDDSLTFKEDIQKAQVSGQGDSLHEHIANLAGSPAI |
| | | KKGILQTVKVVDELVKVMGRHKPENIVIEMARENOTTQKGQKNSRERMK |
| | | RIEEGIKELGSQILKEHPVENTQLQNEKLYLYYLQNGRDMYVDQELDIN |
| | | RLSDYDVDHIVPQSFLKDDSIDNKVLTRSDKNRGKSDNVPSEEVVKKMK |
| | | NYWRQLLNAKLITQRKFDNLTKAERGGLSELDKAGFIKRQLVETRQITK |
| | | HVAQILDSRMNTKYDENDKLIREVKVITLKSKLVSDFRKDFQFYKVREI |
| | | NNYHHAHDAYLNAVVGTALIKKYPKLESEFVYGDYKVYDVRKMIAKSEQ |
| | | EIGKATAKYFFYSNIMNFFKTEITLANGEIRKRPLIETNGETGEIVWDK |
| | | GRDFATVRKVLSMPQVNIVKKTEVQTGGFSKESILPKRNSDKLIARKKD |
| | | WDPKKYGGFDSPTVAYSVLVVAKVEKGKSKKLKSVKELLGITIMERSSF |
| | | EKNPIDFLEAKGYKEVKKDLIIKLPKYSLFELENGRKRMLASAGELQKG |
| | | NELALPSKYVNFLYLASHYEKLKGSPEDNEQKOLFVEQHKHYLDEIIEQ |
| | | ISEFSKRVILADANLDKVLSAYNKHRDKPIREQAENIIHLFTLTNLGAP |
| | | AAFKYFDTTIDRKRYTSTKEVLDATLIHQSITGLYETRIDLSQLGGDEG |
| | | ADKRTADGSEFESPKKKRKV (SEQ ID NO: 1181) |
| 5′ UTR | mRNA | AGGAAAu′AAGAGAGAAAAGAAGAGu′AAGAAGAAAu′Au′AAGAGCCA |
| | | CC (SEQ ID NO: 1182) |
| TadA | mRNA | Au′GAGCGAGGu′GGAGu′u′CAGCCACGAGu′ACu′GGAu′GCGGCAC |
| | | GCCCu′GACCCu′GGCCAAGCGGGCCCGGGACGAGCGGGAGGu′GCCCG |
| | | u′GGGCGCCGu′GCu′GGu′GCu′GAACAACCGGGu′GAu′CGGCGAGG |
| | | GCu′GGAACCGGGCCAu′CGGCCu′GCACGACCCCACCGCCCACGCCGA |
| | | GAu′CAu′GGCCCu′GCGGCAGGGCGGCCu′GGu′GAu′GCAGAACu′A |
| | | CCGGCu′GAu′CGACGCCACCCu′Gu′ACGu′GACCu′u′CGAGCCCu′ |
| | | GCGu′GAu′Gu′GCGCCGGCGCCAu′GAu′CCACAGCCGGAu′CGGCCG |
| | | GGu′GGu′Gu′u′CGGCGu′GCGGAACGCCAAGACCGGCGCCGCCGGCA |
| | | GCCu′GAu′GGACGu′GCu′GCACCACCCCGGCAu′GAACCACCGGGu′ |
| | | GGAGAu′CACCGAGGGCAu′CCu′GGCCGACGAGu′GCGCCGCCCu′GC |
| | | u′Gu′GCCGGu′u′Cu′u′CCGGAu′GCCCCGGCGGGu′Gu′u′CAACG |
| | | CCCAGAAGAAGGCCCAGAGCAGCACCGAC (SEQ ID NO: 1183) |
| | protein | MSEVEFSHEYWMRHALTLAKRARDEREVPVGAVLVLNNRVIGEGWNRAI |
| | | GLHDPTAHAEIMALRQGGLVMQNYRLIDATLYVTFE PCVMCAGAMIHSR |
| | | IGRVVFGVRNAKTGAAGSLMDVLHHPGMNHRVEITEGILADECAALLCR |
| | | FFRMPRRVFNAQKKAQSSTD (SEQ ID NO: 1184) |
| Linker | mRNA | AGCGGCGGCAGCAGCGGCGGCAGCAGCGGCAGCGAGACACCCGGCACCA |
| between | | GCGAGAGCGCCACCCCCGAGAGCAGCGGCGGCAGCAGCGGCGGCAGC |
| TadA | | (SEQ ID NO: 1185) |
| and Cas9 | protein | SGGSSGGSSGSETPGTSESATPESSGGSSGGS (SEQ ID NO: 357) |
| nickase | | |
| Cas9 | mRNA | GACAAGAAGu′ACAGCAu′CGGCCu′GGCCAu′CGGCACCAACAGCGu′ |
| nickase | | GGGCu′GGGCCGu′GAu′CACCGACGAGu′ACAAGGu′GCCCAGCAAGA |
| | | AGu′u′CAAGGu′GCu′GGGCAACACCGACCGGCACAGCAu′CAAGAAG |
| | | AACCu′GAu′CGGCGCCCu′GCu′Gu′u′CGACAGCGGCGAGACAGCCG |
| | | AGGCCACCCGGCu′GAAGCGGACCGCCCGGCGGCGGu′ACACCCGGCGG |
| | | AAGAACCGGAu′Cu′GCu′ACCu′GCAGGAGAu′Cu′u′CAGCAACGAG |
| | | Au′GGCCAAGGu′GGACGACAGCu′u′Cu′u′CCACCGGCu′GGAGGAG |
| | | AGCu′u′CCu′GGu′GGAGGAGGACAAGAAGCACGAGCGGCACCCCAu′ |
| | | Cu′u′CGGCAACAu′CGu′GGACGAGGu′GGCCu′ACCACGAGAAGu′A |
| | | CCCCACCAu′Cu′ACCACCu′GCGGAAGAAGCu′GGu′GGACAGCACCG |
| | | ACAAGGCCGACCu′GCGGCu′GAu′Cu′ACCu′GGCCCu′GGCCCACAu |
| | | ′GAu′CAAGu′u′CCGGGGCCACu′u′CCu′GAu′CGAGGGCGACCu′G |
| | | AACCCCGACAACAGCGACGu′GGACAAGCu′Gu′u′CAu′CCAGCu′GG |
| | | u′GCAGACCu′ACAACCAGCu′Gu′u′CGAGGAGAACCCCAu′CAACGC |
| | | CAGCGGCGu′GGACGCCAAGGCCAu′CCu′GAGCGCCCGGCu′GAGCAA |
| | | GAGCCGGCGGCu′GGAGAACCu′GAu′CGCCCAGCu′GCCCGGCGAGAA |
| | | GAAGAACGGCCu′Gu′u′CGGCAACCu′GAu′CGCCCu′GAGCCu′GGG |
| | | CCu′GACCCCCAACu′u′CAAGAGCAACu′u′CGACCu′GGCCGAGGAC |
| | | GCCAAGCu′GCAGCu′GAGCAAGGACACCu′ACGACGACGACCu′GGAC |
| | | AACCu′GCu′GGCCCAGAu′CGGCGACCAGu′ACGCCGACCu′Gu′u′C |
| | | Cu′GGCCGCCAAGAACCu′GAGCGACGCCAu′CCu′GCu′GAGCGACAu |
| | | ′CCu′GCGGGu′GAACACCGAGAu′CACCAAGGCCCCCCu′GAGCGCCA |
| | | GCAu′GAu′CAAGCGGu′ACGACGAGCACCACCAGGACCu′GACCCu′G |
| | | Cu′GAAGGCCCu′GGu′GCGGCAGCAGCu′GCCCGAGAAGu′ACAAGGA |
| | | GAu′Cu′u′Cu′u′CGACCAGAGCAAGAACGGCu′ACGCCGGCu′ACAu |
| | | ′CGACGGCGGCGCCAGCCAGGAGGAGu′u′Cu′ACAAGu′u′CAu′CAA |
| | | GCCCAu′CCu′GGAGAAGAu′GGACGGCACCGAGGAGCu′GCu′GGu′G |
| | | AAGCu′GAACCGGGAGGACCu′GCu′GCGGAAGCAGCGGACCu′u′CGA |
| | | CAACGGCAGCAu′CCCCCACCAGAu′CCACCu′GGGCGAGCu′GCACGC |
| | | CAu′CCu′GCGGCGGCAGGAGGACu′u′Cu′ACCCCu′u′CCu′GAAGG |
| | | ACAACCGGGAGAAGAu′CGAGAAGAu′CCu′GACCu′u′CCGGAu′CCC |
| | | Cu′ACu′ACGu′GGGCCCCCu′GGCCCGGGGCAACAGCCGGu′u′CGCC |
| | | u′GGAu′GACCCGCAAGAGCGAGGAGACAAu′CACCCCCu′GGAACu′u |
| | | ′CGAGGAGGu′GGu′GGACAAGGGCGCCAGCGCCCAGAGCu′u′CAu′C |
| | | GAGCGGAu′GACCAACu′u′CGACAAGAACCu′GCCCAACGAGAAGGu′ |
| | | GCu′GCCCAAGCACAGCCu′GCu′Gu′ACGAGu′ACu′u′CACCGu′Gu |
| | | ′ACAACGAGCu′GACCAAGGu′GAAGu′ACGu′GACCGAGGGCAu′GCG |
| | | GAAGCCCGCCu′u′CCu′GAGCGGCGAGCAGAAGAAGGCCAu′CGu′GG |
| | | ACCu′GCu′Gu′u′CAAGACCAACCGGAAGGu′GACCGu′GAAGCAGCu |
| | | ′GAAGGAGGACu′ACu′u′CAAGAAGAu′CGAGu′GCu′u′CGACAGCG |
| | | u′GGAGAu′CAGCGGCGu′GGAGGACCGGu′u′CAACGCCAGCCu′GGG |
| | | CACCu′ACCACGACCu′GCu′GAAGAu′CAu′CAAGGACAAGGACu′u′ |
| | | CCu′GGACAACGAGGAGAACGAGGACAu′CCu′GGAGGACAu′CGu′GC |
| | | u′GACCCu′GACCCu′Gu′u′CGAGGACCGGGAGAu′GAu′CGAGGAGC |
| | | GGCu′GAAGACCu′ACGCCCACCu′Gu′u′CGACGACAAGGu′GAu′GA |
| | | AGCAGCu′GAAGCGGCGGCGGu′ACACCGGCu′GGGGCCGGCu′GAGCC |
| | | GGAAGCu′GAu′CAACGGCAu′CCGGGACAAGCAGAGCGGCAAGACCAu |
| | | ′CCu′GGACu′u′CCu′CAAGAGCGACGGCu′u′CGCCAACCGGAACu′ |
| | | u′CAu′GCAGCu′GAu′CCACGACGACAGCCu′GACCu′u′CAAGGAGG |
| | | ACAu′CCAGAAGGCCCAGGu′GAGCGGCCAGGGCGACAGCCu′GCACGA |
| | | GCACAu′CGCCAACCu′GGCCGGCAGCCCCGCCAu′CAAGAAGGGCAU′ |
| | | CCu′GCAGACCGu′GAAGGu′GGu′GGACGAGCu′GGu′GAAGGu′GAu |
| | | ′GGGCCGGCACAAGCCCGAGAACAu′CGu′GAu′CGAGAu′GGCCCGGG |
| | | AGAACCAGACCACCCAGAAGGGCCAGAAGAACAGCCGGGAGCGGAu′GA |
| | | AGCGGAu′CGAGGAGGGCAu′CAAGGAGCu′GGGCAGCCAGAu′CCu′G |
| | | AAGGAGCACCCCGu′GGAGAACACCCAGCu′GCAGAACGAGAAGCu′Gu |
| | | ′ACCu′Gu′ACu′ACCu′GCAGAACGGCCGGGACAu′Gu′ACGu′GGAC |
| | | CAGGAGCu′GGACAu′CAACCGGCu′GAGCGACu′ACGACGu′GGACCA |
| | | CAu′CGu′GCCCCAGAGCu′u′CCu′GAAGGACGACAGCAu′CGACAAC |
| | | AAGGu′GCu′GACCCGGAGCGACAAGAACCGGGGCAAGAGCGACAACGu |
| | | ′GCCCAGCGAGGAGGu′GGu′GAAGAAGAu′GAAGAACu′ACu′GGCGG |
| | | CAGCu′GCu′GAACGCCAAGCu′GAu′CACCCAGCGGAAGu′u′CGACA |
| | | ACCu′GACCAAGGCCGAGCGGGGCGGCCu′GAGCGAGCu′GGACAAGGC |
| | | CGGCu′u′CAu′CAAGCGGCAGCu′GGu′GGAGACACGGCAGAUu′CACC |
| | | AAGCACGu′GGCCCAGAu′CCu′GGACAGCCGGAu′GAACACCAAGu′A |
| | | CGACGAGAACGACAAGCu′GAu′CCGGGAGGu′GAAGGu′GAu′CACCC |
| | | u′CAAGAGCAAGCu′GGu′GAGCGACu′u′CCGGAAGGACu′u′CCAGu |
| | | ′u′Cu′ACAAGGu′GCGGGAGAu′CAACAACu′ACCACCACGCCCACGA |
| | | CGCCu′ACCu′GAACGCCGu′GGu′GGGCACCGCCCu′GAu′CAAGAAG |
| | | u′ACCCCAAGCu′GGAGAGCGAGu′u′CGu′Gu′ACGGCGACu′ACAAG |
| | | Gu′Gu′ACGACGu′GCGGAAGAu′GAu′CGCCAAGAGCGAGCAGGAGAU |
| | | ′CGGCAAGGCCACCGCCAAGu′ACu′u′Cu′u′Cu′ACAGCAACAu′CA |
| | | u′GAACu′u′Cu′u′CAAGACCGAGAu′CACCCu′GGCCAACGGCGAGA |
| | | u′CCGGAAGCGGCCCCu′GAu′CGAGACAAACGGCGAGACAGGCGAGAU |
| | | ′CGu′Gu′GGGACAAGGGCCGGGACu′u′CGCCACCGu′GCGGAAGGu′ |
| | | GCu′GAGCAu′GCCCCAGGu′GAACAu′CGu′GAAGAAGACCGAGGu′G |
| | | CAGACCGGCGGCu′u′CAGCAAGGAGAGCAu′CCu′GCCCAAGCGGAAC |
| | | AGCGACAAGCu′GAu′CGCCCGGAAGAAGGACu′GGGACCCCAAGAAGU |
| | | ′ACGGCGGCu′u′CGACAGCCCCACCGu′GGCCu′ACAGCGu′GCu′GG |
| | | u′GGu′GGCCAAGGu′GGAGAAGGGCAAGAGCAAGAAGCu′CAAGAGCG |
| | | u′GAAGGAGCu′GCu′GGGCAu′CACCAu′CAu′GGAGCGGAGCAGCu′ |
| | | u′CGAGAAGAACCCCAu′CGACu′u′CCu′GGAGGCCAAGGGCu′ACAA |
| | | GGAGGu′GAAGAAGGACCu′GAu′CAu′CAAGCu′GCCCAAGu′ACAGC |
| | | Cu′Gu′u′CGAGCu′GGAGAACGGCCGGAAGCGGAu′GCu′GGCCAGCG |
| | | CCGGCGAGCu′GCAGAAGGGCAACGAGCu′GGCCCu′GCCCAGCAAGu′ |
| | | ACGu′GAACu′u′CCu′Gu′ACCu′GGCCAGCCACu′ACGAGAAGCu′G |
| | | AAGGGCAGCCCCGAGGACAACGAGCAGAAGCAGCu′Gu′u′CGu′GGAG |
| | | CAGCACAAGCACu′ACCu′GGACGAGAu′CAu′CGAGCAGAu′CAGCGA |
| | | Gu′u′CAGCAAGCGGGu′GAu′CCu′GGCCGACGCCAACCu′GGACAAG |
| | | Gu′GCu′GAGCGCCu′ACAACAAGCACCGGGACAAGCCCAu′CCGGGAG |
| | | CAGGCCGAGAACAu′CAu′CCACCu′Gu′u′CACCCu′GACCAACCu′G |
| | | GGCGCCCCCGCCGCCu′u′CAAGu′ACu′u′CGACACCACCAu′CGACC |
| | | GGAAGCGGu′ACACCAGCACCAAGGAGGu′GCu′GGACGCCACCCu′GA |
| | | u′CCACCAGAGCAu′CACCGGCCu′Gu′ACGAGACACGGAu′CGACCu′ |
| | | GAGCCAGCu′GGGCGGCGAC (SEQ ID NO: 1186) |
| | protein | DKKYSIGLAIGTNSVGWAVITDEYKVPSKKFKVLGNTDRHSIKKNLIGA |
| | | LLFDSGETAEATRLKRTARRRYTRRKNRICYLQEIFSNEMAKVDDSFFH |
| | | RLEESFLVEEDKKHERHPIFGNIVDEVAYHEKYPTIYHLRKKLVDSTDK |
| | | ADLRLIYLALAHMIKFRGHFLIEGDLNPDNSDVDKLFIQLVQTYNQLFE |
| | | ENPINASGVDAKAILSARLSKSRRLENLIAQLPGEKKNGLFGNLIALSL |
| | | GLTPNFKSNFDLAEDAKLQLSKDTYDDDLDNLLAQIGDQYADLFLAAKN |
| | | LSDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVKL |
| | | NREDLLRKORTFDNGSIPHQIHLGELHAILRRQEDFYPFLKDNREKIEK |
| | | ILTFRIPYYVGPLARGNSRFAWMTRKSEETITPWNFEEVVDKGASAQSF |
| | | IERMTNFDKNLPNEKVLPKHSLLYEYFTVYNELTKVKYVTEGMRKPAFL |
| | | SGEQKKAIVDLLFKTNRKVTVKOLKEDYFKKIECFDSVEISGVEDRFNA |
| | | SLGTYHDLLKIIKDKDFLDNEENEDILEDIVLTLTLFEDREMIEERLKT |
| | | YAHLFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIKKG |
| | | ILQTVKVVDELVKVMGRHKPENIVIEMARENQTTQKGQKNSRERMKRIE |
| | | EGIKELGSQILKEHPVENTQLQNEKLYLYYLQNGRDMYVDQELDINRLS |
| | | DYDVDHIVPQSFLKDDSIDNKVLTRSDKNRGKSDNVPSEEVVKKMKNYW |
| | | ROLLNAKLITQRKFDNLTKAERGGLSELDKAGFIKRQLVETRQITKHVA |
| | | QILDSRMNTKYDENDKLIREVKVITLKSKLVSDFRKDFQFYKVREINNY |
| | | HHAHDAYLNAVVGTALIKKYPKLESEFVYGDYKVYDVRKMIAKSEQEIG |
| | | KATAKYFFYSNIMNFFKTEITLANGEIRKRPLIETNGETGEIVWDKGRD |
| | | FATVRKVLSMPQVNIVKKTEVQTGGFSKESILPKRNSDKLIARKKDWDP |
| | | KKYGGFDSPTVAYSVLVVAKVEKGKSKKLKSVKELLGITIMERSSFEKN |
| | | PIDFLEAKGYKEVKKDLIIKLPKYSLFELENGRKRMLASAGELQKGNEL |
| | | ALPSKYVNFLYLASHYEKLKGSPEDNEQKOLFVEQHKHYLDEIIEQISE |
| | | FSKRVILADANLDKVLSAYNKHRDKPIREQAENIIHLFTLTNLGAPAAF |
| | | KYFDTTIDRKRYTSTKEVLDATLIHQSITGLYETRIDLSQLGGD (SEQ |
| | | ID NO: 1187) |
| Linker | mRNA | GAGGGCGCCGAC (SEQ ID NO: 1188) |
| between | Protein | EGAD (SEQ ID NO: 1189) |
| Cas9 | | |
| nickase | | |
| and NLS | | |
| Nuclear | mRNA | AAGCGGACCGCCGACGGCAGCGAGu′u′CGAGAGCCCCAAGAAGAAGCG |
| localization | | GAAGGu′Gu′GA (SEQ ID NO: 1190) |
| sequence | Protein | KRTADGSEFESPKKKRKV (SEQ ID NO: 190) |
| (NLS) | | |
| 3′ UTR | mRNA | GCGGCCGCu′u′AAu′u′AAGCu′GCCu′u′Cu′GCGGGGCu′u′GCCu |
| | | ′u′Cu′GGCCAu′GCCCu′u′Cu′u′Cu′Cu′CCCu′u′GCACCu′Gu′ |
| | | ACCu′Cu′u′GGu′Cu′u′u′GAAu′AAAGCCu′GAGu′AGGAAGu′Cu |
| | | ′AGA (SEQ ID NO: 1191) |

The mutations at amino acid positions 691 and 1135 of the nCas9 component and their corresponding nucleotide sequences are indicated as bold and underlined.
u′: N¹-methylpseudouridine
The first nucleotide in the 5′ UTR has a 2′-O-methyl modification.

Other ABE variants may be employed to effect editing of human TTR gene. Examples of such ABE variants are described International Patent Application PCT/US21/26729, filed on Apr. 9, 2021, entitled BASE EDITING OF PCSK9 AND METHODS OF USING SAME FOR TREATMENT OF DISEASE, and naming Verve Therapeutics, Inc. as the applicant.

### Example 3: In Vivo Non-human primate (NHP) Base Editing of TTR Gene

In this example, NHP surrogate sgRNAs (GA519 and GA520), corresponding to the human GA457 and GA460 sgRNAs described above, were prepared, and formulated with previously described ABE8.8 mRNA, encapsulated in lipid nanoparticles (LNPs), and intravenously dosed to NHPs. The study involved two distinct aspects.

The first aspect of the NHP in vivo study involved evaluating LNP1 and LNP2, which differed only in that LNP1 was formulated to encapsulate GA519 and ABE8.8 mRNA whereas LNP2 was formulated to encapsulate GA520 and ABE8.8 mRNA. The second aspect of the study involved formulating and evaluating a third LNP(LNP3). LNP3, like LNP1, was formulated to encapsulate GA519 and ABE8.8 mRNA. However, LNP 3 differed from LNP1 in that LNP3 included a GalNAc moiety constituent. In each aspect of the study, base editing efficiency, TTR protein expression, safety profiles, and pharmacokinetics were evaluated at multiple times post-infusion of the NHPs, as is further detailed below and illustrated in the accompanying figures.

### Part a: In Vivo NHP Evaluation of GA519 and GA520 Using Non-GaINAc LNPs

### LNP Preparation

In this first aspect of the NHP study, two LNPs (LNP1 and LNP2) were formulated, with LNP1 encapsulating GA519 and ABE8.8 mRNA and LNP2 encapsulate GA520 and ABE8.8 mRNA. The constituents of each of the LNPs are comprised of an ionizable amino lipid (iLipid), a neutral helper lipid, a PEG-Lipid and a sterol lipid as described in and at the ratios indicated in Table 19 below.

TABLE 19
LNP1/LNP2 Components

| LNP | | | Mol |
|---|---|---|---|
| Component | Lipid names | Lipid structure | % |
| Amino lipid (iLipid) | 3-((4,4-bis(octyloxy)butanoyl)oxy)-2- ((((3- (diethylamino)propoxy)carbonyl)oxy)meth- yl)propyl(9Z,12Z)-octadeca-9,12- dienoate* | 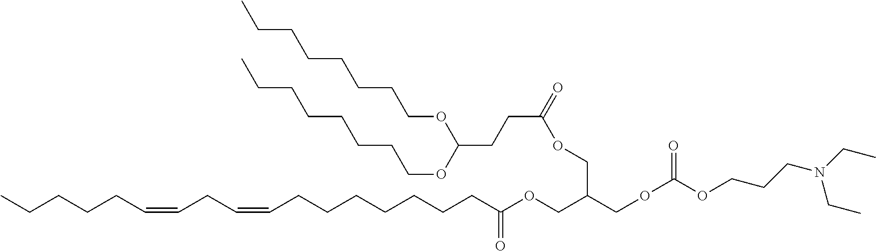 [SMILES: CCCCC/C=C\C/C=C\CCCCCCCC(=O)OCC(COC(=O)CCC(OCCCCCCCC)OCCCCCCCC)COC(=O)OCCCN(CC)CC] | 50 |
| Neutral helper lipid | 1,2-distearoyl-sn-glycero-3- phosphocholine (DSPC) | 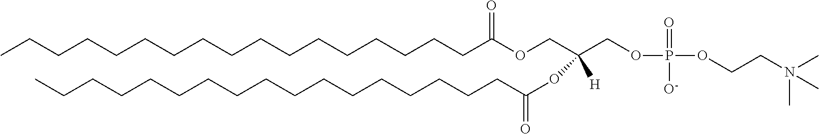 | 9 |
| PEG-lipid | 1,2-dimyristoyl-rac-glycero-3- methoxypolyethylene glycol-2000 (PEG₂₀₀₀-DMG) | 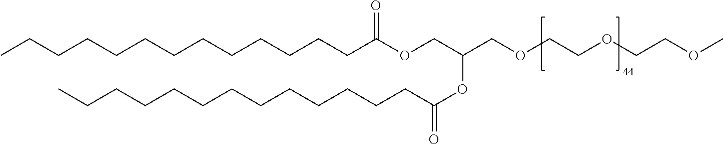 | 3 |
| Sterol lipid | Cholesterol | 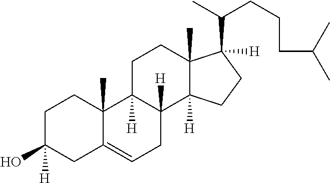 | 38 |

*described in International Published Patent Application WO 2015/095340 A1

It should be understood that the lipids in Table 19 may be substituted for other suitable lipids in the listed class. In some embodiments, for example, the LNP comprises the amino lipid VL422 described in the International published patent application WO 2022/060871 A¹. For example, the amino lipid may be VL422, or a pharmaceutically acceptable salt or solvate thereof:

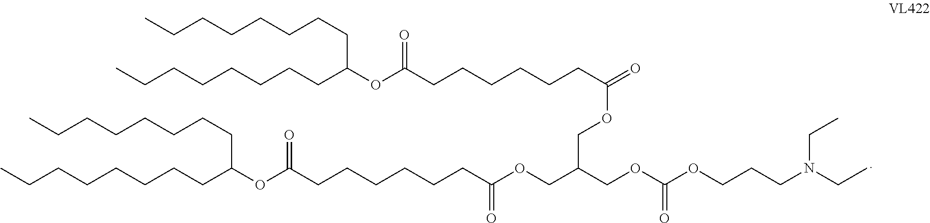 [SMILES: CCCCCCCCC(CCCCCCCC)OC(=O)CCCCCCC(=O)OCC(COC(=O)CCCCCCC(=O)OC(CCCCCCCC)CCCCCCCC)COC(=O)OCCCN(CC)CC]

It should be further understood that the mol % of lipids in Table 19 may be adjusted and that the mol % included in Table 19 are targeted excipient percentages of the LNP, which is intended to represent the aggregate mol % of all the LNPs formulated in a given batch and that specific LNPs within a batch may have varying mol %. Thus, it is contemplated herein that the mol % of one or more, or all of the LNP components set forth in Table 19 may be adjusted, for example, by +/−1-5%, +/−5-10%, or +/−10%-20%. It is further contemplated herein that the mol % of one or more, or all of the LNP components set forth in Table 19 with respect to a specific LNP formulated in a given batch of LNPs formulated in accordance with desired target excipient percentages, may vary from the targeted mol %, for example, by +/−1-5%, +/−5-10%, or +/−10%-20%, or even greater than +/−20%. Further, it should be understood that additional LNP components, including non-lipid components, may be added to the LNP components set-forth in Table 19. As set forth in Table 20, LNP 1 was formulated with sgRNA GA519 and LNP2 was formulated with GA520, which correspond respectively to the sgRNA GA457 and GA460, previously described. GA519 and GA520 were chemically synthesized and the sequences and chemical modifications of GA519 and GA520 are specified in Table 20.

TABLE 20
GA519 and GA520 TTR Gene Targeted Guides

| | | | | Equivalent | |
|---|---|---|---|---|---|
| | gRNA | | Protospacer | Human | gRNA Sequence (5′-3′) |
| LNP | ID | Species | (5′-3′) | gRNA | (spacers are in bold) |
| 1 | GA519 | Cyno | GCCATCCTGC | GA457 | gscscsAUCCUGCCAAGAAC |
| | | | CAAGAACGAG | | GAGgUUUUAGagcuaGaaau |
| | | | (SEQ ID NO: | | agcaaGUUaAaAuAaggcua |
| | | | 471) | | GUccGUUAucAAcuuGaaaa |
| | | | | | agugGcaccgagucggugcu |
| | | | | | ususus (SEQ ID NO: 1044) |
| 2 | GA520 | Cyno | TATAGGAAAA | GA460 | usasusAGGAAAACCAGUGA |
| | | | CCAGTGAGTC | | GUCgUUUUAGagcuaGaaau |
| | | | (SEQ ID NO: | | agcaaGUUaAaAuAaggcua |
| | | | 469) | | GUccGUUAucAAcuuGaaaa |
| | | | | | agugGcaccgagucggugcu |
| | | | | | ususus (SEQ ID NO: 497) |

Letters in the sequences: A = adenosine; C = cytidine; G = guanosine; U = uridine; a = 2′-O-methyladenosine; c = 2′-O-methylcytidine; g = 2′-O-methylguanosine; u = 2′-O-methyluridine; s = phosphorothioate (PS) backbone linkage. C = nucleotide that differs in NHP from human TTR sequence. Bold type in gRNA sequence denotes spacer sequence corresponding to Protospacer.

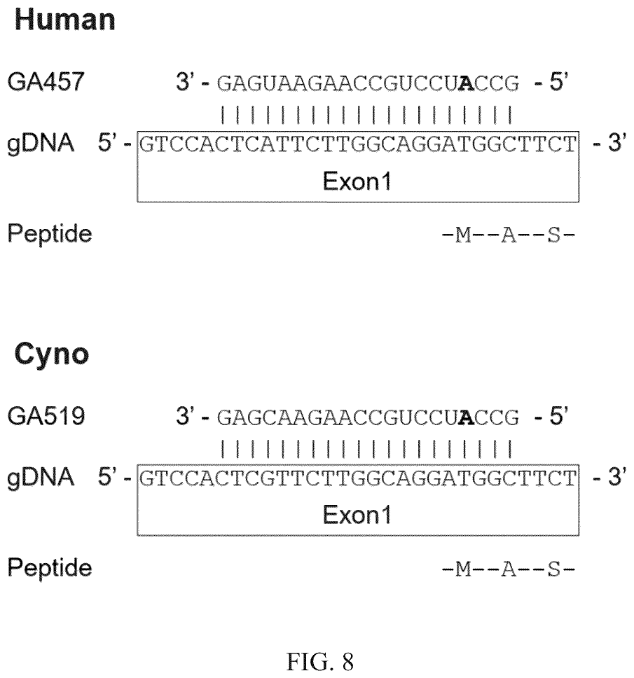

Notably as compared to GA457, GA519 hybridizes between positions 50,681,581 to 50,681,603 in exon 1 of the reference cynomolgus monkey genome (macFas5) and edits the adenosine at position 50,681,584 resulting in disruption of the full length TTR protein sequence by converting a methionine to a threonine amino acid and prohibiting protein translation (FIG. 8). GA519 is the cynomolgus surrogate of the human GA457 gRNA and maps to the analogous region of the human TTR locus as in FIG. 4 as previously described. The cynomolgus GA519 gRNA differs from GA457 by a single nucleotide at position 17 of the protospacer and is highlighted with an underline in Protospacer column of Table 20. Furthermore, GA519 and GA457 differ from one another in that the tracr region of GA519 incorporates chemical modifications (detailed in Table 20). The chemical modifications are designed for, or capable of, improving in vivo stability.

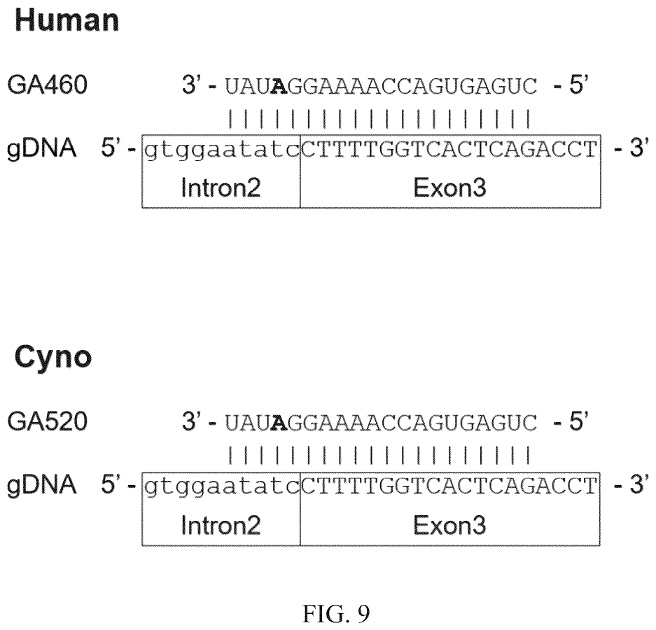

Similarly, as compared to GA460, GA520 hybridizes between positions 50,678,305 to 50,678,327 of exon 3 of the reference cynomolgus monkey genome (macFas5) and edits the adenosine at position 50,678,324 resulting in splicing acceptor disruption producing a truncated non-functional TTR protein (FIG. 9). The protospacer region for GA520 is identical to the human GA460 and maps to the analogous region of the human TTR locus as in FIG. 4 as previously described. GA520 and GA460 differ in the tracr region and incorporate chemical modifications, as detailed in the table above, that are designed for, or capable of, improving in vivo stability.

For reference, the targeted nucleotide for base editing is highlighted in bold in FIGS. 8 and 9. FIGS. 8 and 9 also identify the location of the spacer of GA519 and GA520 relative to the TTR gene as previously described.

LNP 1 and LNP2 were formulated using ABE 8.8 mRNA and GA519 and GA520, respectively, with an sgRNA: mRNA weight ratio of 1:1. In other words, the LNPs were formulated with an equal amount by weight of guide RNA as mRNA. The resulting LNPs encapsulating the sgRNAs and ABE 8.8 mRNA were filtered using 0.2-micron filters and frozen at −80° C. Physical characteristics of the formulated LNPs are summarized in Table 21.

TABLE 21
LNP1/LNP2 Characterization

| | Average | | RNA |
|---|---|---|---|
| | LNP size | | entrapment |
| LNP | (nm) | PDI | (%) |
| 1 | 68.6 | 0.022 | 95.7 |
| 2 | 68.6 | 0.029 | 96.2 |

PDI is Polydispersity Index

One of ordinary skill in the art would understand that the average LNP size, PDI and RNA entrapment values set forth in Table 21 are subject to measurement error or accuracy. It is also contemplated herein that the LNP size, PDI and RNA entrapment values set forth in Table 21 may be varied by +/−1-5%, +/−5-10%, or +/−10%-20%.

### Nhp Study Design

In this aspect of the study, female cynomolgus monkeys of Cambodian origin were used as study animals. A premedication regimen comprising dexamethasone and H1 and H2 antihistamines was administered to all animals on day-1 (approximately 24 hours prior to dosing) and day 1 (predose), at 30 to 60 minutes prior to test article dose administration. Three monkeys were dosed with LNP1 and 3 monkey were dosed with LNP 2 on day 1 of the study via a single IV infusion at a dose level of 3 mg of combined sgRNA and mRNA per kg of animal body weight and at a dose volume of 6 mL/kg (n=3/group).

Blood samples were collected from all animals predose for baseline measurement and post-dose at various time points on days 1 through 15 to assess biomarkers, cytokines, plasma iLipid and PEG-Lipid pharmacokinetics, and serum safety parameters.

Necropsies were performed on all animals at day 16. Liver biopsy samples were collected to assess TTR gene editing.

### Analysis of Editing Efficiency

The amount of gene editing in the liver was evaluated by next-generation sequencing

(NGS) of targeted polymerase chain reaction (PCR) amplicons at the TTR target site derived from genomic DNA extracted from the liver of the animal using the method described previously (Musunuru et al, Nature 593, no. 7859 (May 2021): 429-34. doi: 10.1038/s41586-021-03534-y). Percent editing was reported as the percent of all reads containing a nonreference allele at the target adenine.

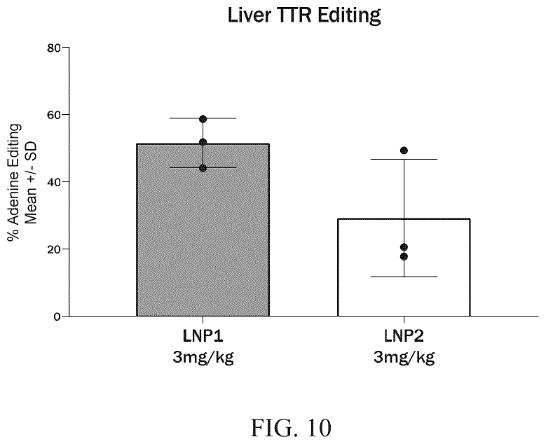

FIG. 10 illustrates TTR editing efficiency of LNP1 as compared to LNP2. Notably, as illustrated in FIG. 10, the average hepatic TTR editing efficiency is higher in NHP treated with LNP1 (52%) compared to LNP2 (29%).

### Quantification of TTR Protein Expression in Serum

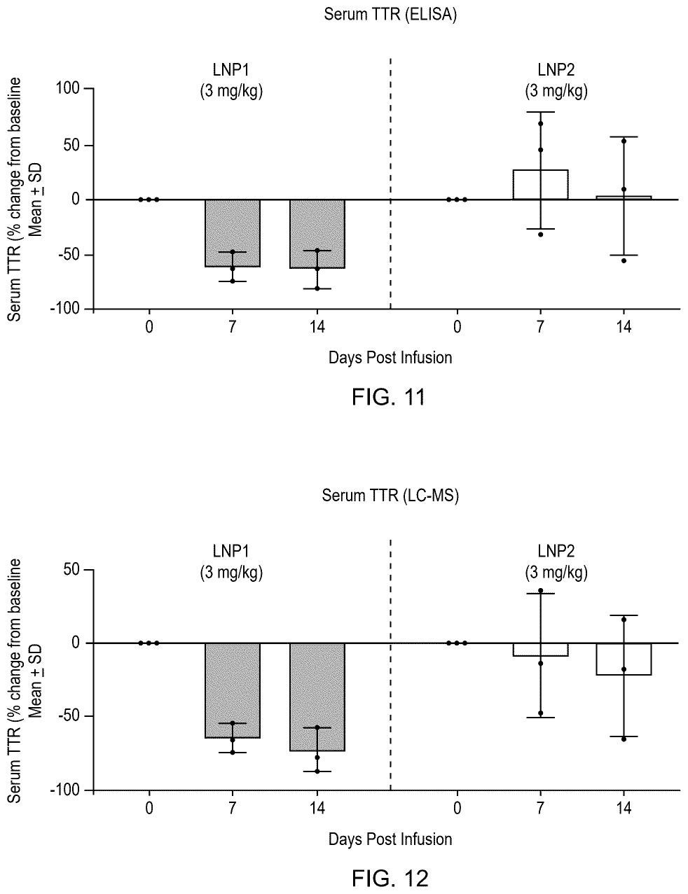

Serum was collected from all animals on days-10, −7, −5 pre-infusion and days 7, and 14 post LNP infusion for TTR protein analysis. Serum TTR was quantified using two methods. TTR protein levels were initially quantified using a custom TTR sandwich ELISA with the data obtained from that analysis presented in FIG. 11. Values for day −10, −7, and −5 were averaged to obtain the baseline value. Notably, LNP1 treated animals showed greater liver TTR editing, also showed greater plasma TTR reductions (−63% change from baseline on Day 14) when compared to LNP2 treated animals (3% change from baseline on Day 14). TTR protein collected from serum were also quantitated using liquid chromatography mass-spectrometry (LC-MS), in which four unique serum TTR peptide fragments were quantitated from each sample time point and the average of the results is reported. LC-MS serum TTR quantitation analysis using LC-MS is set forth in FIG. 12 and was notably consistent with the data obtained from the ELISA quantification in that it also demonstrated that LNP1 showed greater plasma TTR reductions (−73% change from baseline on day 14) when compared to LNP2 (−21% change from baseline on day 14).

Thus, as illustrated in FIGS. 10, 11 and 12, infusion of LNP1 and LNP2 in NHPs resulted in editing of the TTR gene in the liver, with LNP1 demonstrating greater editing than LNP2. The greater editing of LNP1 NHPs corresponded to a commensurate increase in the reduction in serum TTR concentrations in serum.

### Safety Analysis

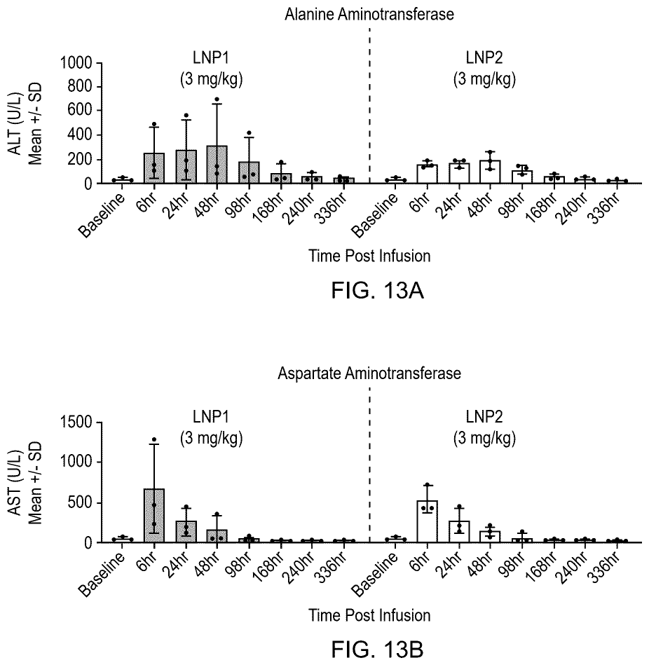

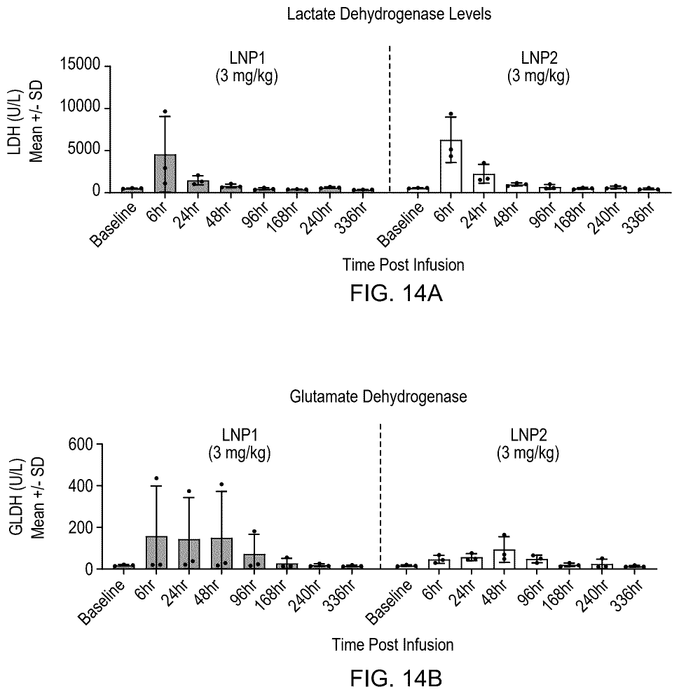

Blood serum was collected from all animals at day-10, −7, −5 pre-infusion and 6, 24, 48, 96, 168, 240, and 336 hours post LNP infusion for safety analysis and specifically directed to observing changes in liver enzymes and cytokine levels. Serum chemistry parameters were directly measured from blood serum samples on a Beckman Coulter AU680 analyzer. Values for day −10, −7, and −5 were averaged to obtain the baseline value. Both LNP1 and LNP2 dosed animals showed transient alanine aminotransferase (FIG. 13A) elevations that peaked at 48 hours post end of infusion and returned to baseline levels 168 hours post end of infusion. Aspartate aminotransferase levels, illustrated in FIG. 13B, were also elevated by both LNP1 and LNP2 treatments, peaking at 6 hours post end of infusion and returning to baseline levels 96 hours post end of infusion. Serum lactate dehydrogenase concentrations, as illustrated in FIG. 14A, and glutamate dehydrogenase concentrations, as illustrated in FIG. 14B, were also found to be elevated shortly following administration of either LNP1 or LNP2 that returned to baseline levels 96-168 hours post end of infusion.

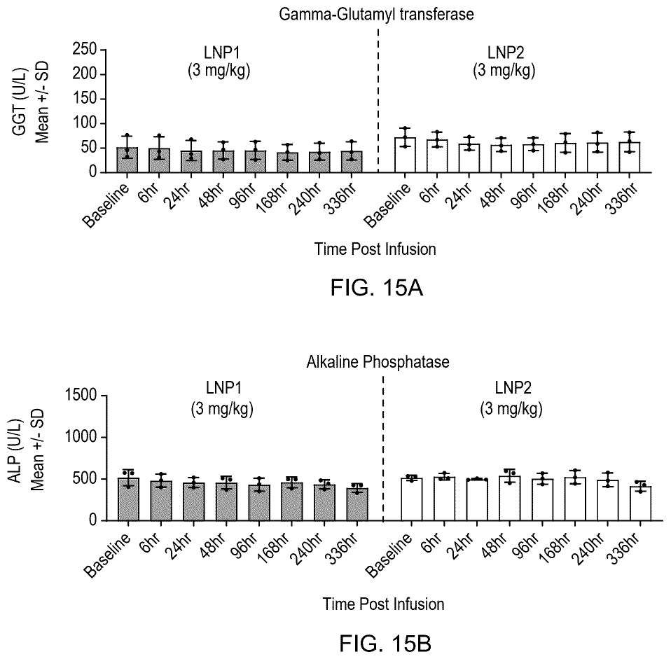

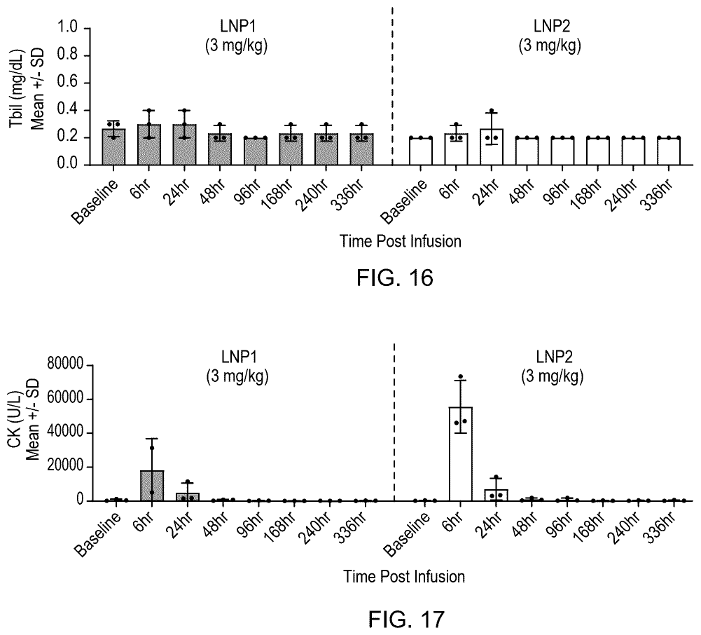

Serum concentrations of gamma-glutamyl transferase, illustrated in FIG. 15A, and alkaline phosphatase, FIG. 15B, were not changed by either LNP1 or LNP2 infusion. In addition, LNP1 and LNP2 treatment did not affect serum total bilirubin concentrations, as illustrated in FIG. 16. LNP1 and LNP2 dosed animals, each also showed elevated serum creatine kinase concentrations, as illustrated in FIG. 17, which in each case peaked at 6 hours and returned fully to baseline levels by 168 hours post end of infusion.

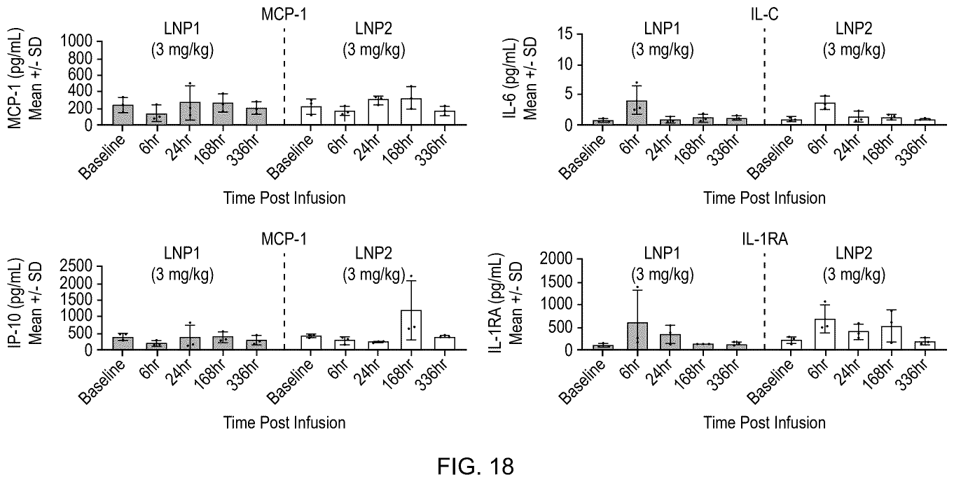

Serum was collected from all animals at day −10, −7, −5 pre-treatment and 24, 168, and 336 hours post LNP infusion for serum cytokine analysis. Cytokines were measured using a multiplexed sandwich immunoassay, where four (MCP-1, IL-6, IP-10, IL-1RA) cytokines are quantitated simultaneously from serum samples using the U-PLEX Biomarker Group 1 (monkey) Assay from Meso Scale Diagnostics (Rockville, MD). Values for day −10, −7, and −5 were averaged to obtain the baseline value. Both LNP1 and LNP2 dosed animals showed elevated serum IL-6 concentrations, as illustrated in FIG. 18, to a similar extent, peaking at 6 hours and returning to baseline by 24 hours post end of infusion. As further illustrated in FIG. 18, both LNP1 and LNP2 dosed animals showed increased serum IL-1RA that peaked at 6 hours and returned fully to baseline by 336 hours. Also, as illustrated in FIG. 18, neither LNP1 nor LNP2 had any measurable significant effect on serum MCP-1 or IP-10 concentrations.

Overall, the analysis of foregoing parameters showed that infusion of either LNP1 and LNP2 in monkeys produced a transient increase in liver enzymes and cytokines that resolves rapidly.

### Pharmacokinetics (PK) Evaluation

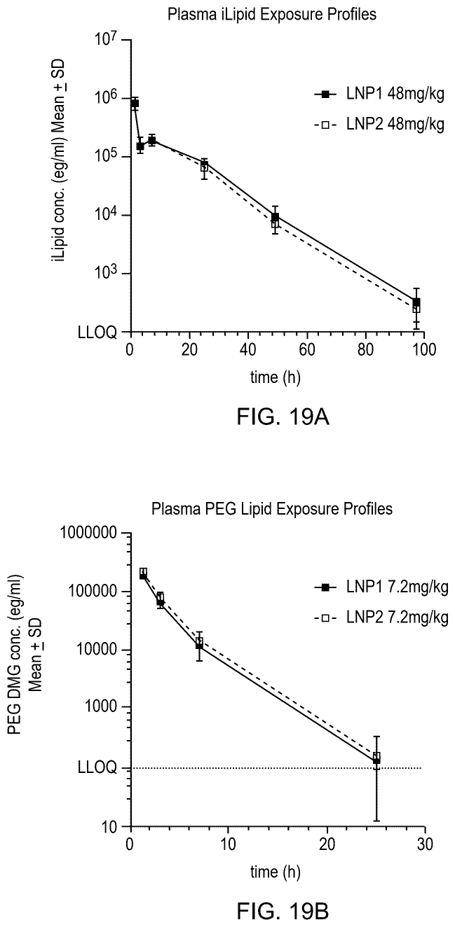

Blood samples were obtained (K2EDTA) for plasma PK analysis and determination of concentrations of the iLipid and PEGLipid excipients that comprised LNP1 and LNP2. After the end of the infusion, plasma samples were collected at 0.25, 2, 6, 24, 48, 96, 168, 240, and 336 hours post LNP infusion. Concentrations of the iLipid and PEG Lipids were measured using qualified LC-MS assays and are shown in FIG. 19A. Timepoints in which the lipids were below the limit of quantitation are not included in the figure. As illustrated in FIG. 19A, serum iLipid concentrations for LNP1 and LNP2 dosed animals continuously declined until approaching lower limit of quantitation (LLOQ) at 96 hour post LNP infusion. Similarly, as illustrated in FIG. 19B, serum PEG-Lipid concentrations for LNP1 and LNP2 dosed animals also rapidly declined reaching an LLOQ at 24 hours post end of infusion.

Part B: In Vivo NHP Evaluation of GA519 with GalNAc LNP

In further evaluation of GA519, an additional LNP(LNP3) was formulated to encapsulate the same GA519 and ABE8.8 mRNA at the 1:1 weight ratio and dosed intravenously to NHPs as previously described. LNP3 differs from LNP1 in that LNP3 was formulated with an additional GalNAc ligand excipient, as described in more detail below.

LNP preparation

The GalNAc LNPs (LNP3) formulated for this aspect of the study were comprised of the same iLipid, neutral helper lipid, PEG-Lipid and sterol lipid as described in connection with LNP1/LNP2, but unlike LNP1/LNP2, LNP3 also is comprised of a GalNAc conjugated lipid. The molar ratios of each constituent component of LNP3 are described in Table 22.

TABLE 22
LNP3 Components

| LNP | | | |
|---|---|---|---|
| Component | Lipid names | Lipid structure | Mol % |
| Amino lipid (iLipid) | 3-((4,4-bis(octyloxy)butanoyl)oxy)- 2-((((3- (diethylamino)propoxy)carbonyl) oxy)methyl)propyl(9Z,12Z)- octadeca-9,12-dienoate* | 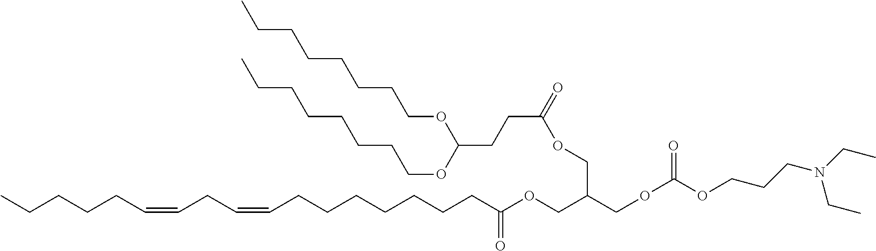 [SMILES: CCCCC/C=C\C/C=C\CCCCCCCC(=O)OCC(COC(=O)CCC(OCCCCCCCC)OCCCCCCCC)COC(=O)OCCCN(CC)CC] | 50 |
| Neutral helper lipid | 1,2-distearoyl-sn-glycero-3- phosphocholine (DSPC) | 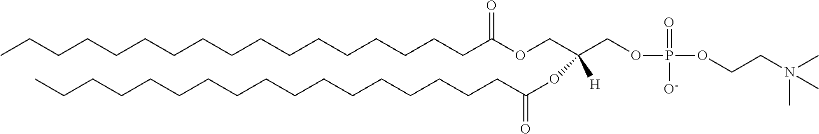 | 9 |
| PEG-lipid | 1,2-dimyristoyl-rac-glycero-3- methoxypolyethylene glycol-2000 (PEG₂₀₀₀-DMG) | 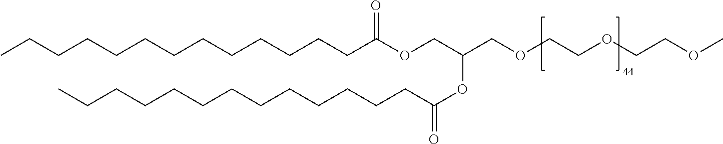 | 3 |
| Sterol lipid | Cholesterol | 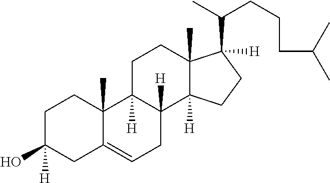 | 37.95 |
| GalNAc- lipid | N²-(PEG-DSG)-N⁶-((C5- GalNAc)amido)-Lys-[bis((C5- GalNAc)propylamido)]amide or DSG-PEG-Lys-tris(GalNAc)** | 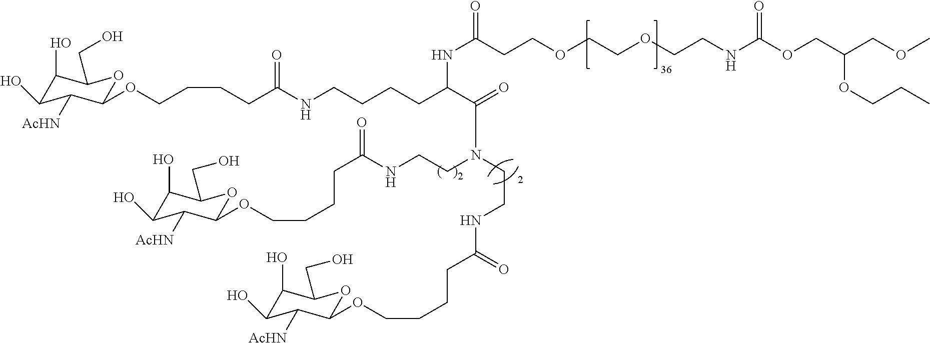 | 0.05 |

*described in International Published Patent Application WO 2015 095340 A1
**described in International Published Patent Application WO 2021/178725 A1

It should be understood that the lipids in Table 22 may be substituted for other suitable lipids in the listed class. For example, the amino lipid may be the following amino lipid, or a salt thereof:

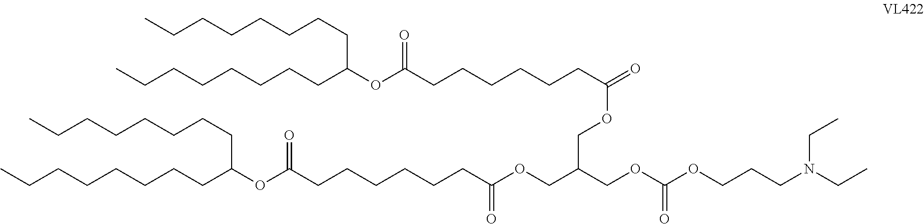 [SMILES: CCCCCCCCC(CCCCCCCC)OC(=O)CCCCCCC(=O)OCC(COC(=O)CCCCCCC(=O)OC(CCCCCCCC)CCCCCCCC)COC(=O)OCCCN(CC)CC]

It should be further understood that the mol % of lipids in Table 20 may be adjusted and that the mol % included in Table 20 are targeted excipient percentages of the LNP, which is intended to represent the aggregate mol % of all the LNPs formulated in a given batch and that specific LNPs within a batch may have varying mol %. Thus, it is contemplated herein that the mol % of one or more, or all of the LNP components set forth in Table 20 may be adjusted, for example, by +/−1-5%, +/−5-10%, or +/−10%-20%. It is further contemplated herein that the mol % of one or more, or all of the LNP components set forth in Table 20 with respect to a specific LNP formulated in a given batch of LNPs formulated in accordance with desired target excipient percentages, may vary from the targeted mol %, for example, by +/−1-5%, +/−5-10%, or +/−10%-20%, or even greater than +/−20%. Further, it should be understood that additional LNP components, including non-lipid components, may be added to the LNP components set-forth in Table 20.

In formulating LNP3, the GalNAc-Lipid was premixed with other LNP excipients referenced in Table 22 prior to in-line mixing with GA519 sgRNA and ABE 8.8 mRNA (at 1:1 weight ratio) to form LNP3. Rajeev et al., WO2021178725, includes a description of the synthesis and characterization of the GalNAc lipid. As with LNP1/LNP2, the resulting GalNAc-LNPs, LNP3, were filtered using 0.2-micron filters and frozen at −80° C. The physical characteristics of the formulated LNP3 is summarized in Table 23.

TABLE 23
LNP3 Characterization.

| | Average | | |
|---|---|---|---|
| | LNP size | | RNA |
| LNP | (nm) | PDI | entrapment |
| 3 | 61.92 | 0.055 | 98.7 |

One of ordinary skill in the art would understand that the average LNP size, PDI and RNA entrapment values set forth in Table 23 are subject to measurement error or accuracy. It is also contemplated herein that the LNP size, PDI and RNA entrapment values set forth in Table 23 may be varied by +/−1-5%, +/−5-10%, or +/−10%-20%.

### NHP Study Design

Male cynomolgus monkeys of Cambodian origin were used in this aspect of the study. A premedication regimen comprising dexamethasone and H1 and H2 antihistamines were administered to all animals on day-1 (approximately 24 hours prior to dosing) and day 1 (predose), between 30 and 60 minutes before test article dose administration. The LNP3 dosing formulations were administered once on day 1 of the study by IV infusion of two groups of 3 monkeys at dose levels of (i)₂ mg of combined sgRNA and mRNA per kg of animal body weight and at a dose volume of 6 mL/kg (n=3/group) for the first group of three monkeys and (ii)₃ mg of combined sgRNA and mRNA per kg of animal body weight and at a dose volume of 6 mL/kg (n=3/group) for the second group of three monkeys.

Blood samples were collected from all animals predose for baseline measurement and post infusion at various time points from days 1 through 35 to assess biomarkers, plasma iLipid and PEG pharmacokinetics, and serum safety parameters.

Necropsies were performed on day 36. Liver tissue samples were collected from all animals to assess TTR gene editing in the liver.

### Analysis of Editing Efficiency

The amount of gene editing in the liver was evaluated by next-generation sequencing (NGS) of targeted polymerase chain reaction (PCR) amplicons at the TTR target site derived from genomic DNA extracted from the liver as described previously (Musunuru et al., Nature 593, no. 7859 (May 2021): 429-34. doi: 10.1038/s41586-021-03534-y). Percent editing was reported as the percent of all reads containing a nonreference allele at the target adenine.

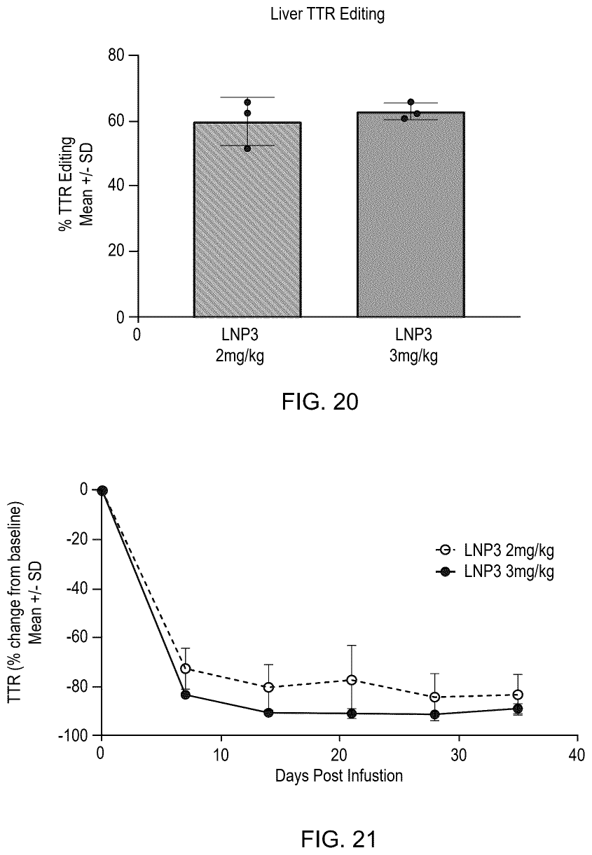

As set forth in FIG. 20, LNP3 led to similar levels of hepatic TTR editing efficiency at 2 mg/kg dosed monkeys (60%) as compared to 3 mg/kg dosed monkeys (63%).

### Quantification of TTR Protein Expression in Serum

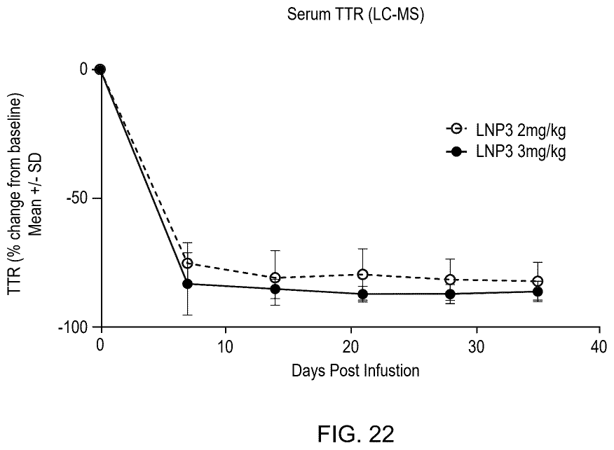

Serum was collected at day-10, −7, −5 pre-infusion and 7, 14, 21, 28, and 35 days post end of infusion for TTR protein analysis. Serum TTR was initially quantified using a custom TTR sandwich ELISA with the data obtained from that analysis presented in FIG. 21. Values for day −10, −7, and −5 were averaged to obtain the baseline value. As illustrated in FIG. 21, both groups of LNP3 dosed animals showed marked reductions in serum TTR protein at the first timepoint (day 7) after dosing. These reductions were maintained for the duration of the study, reaching maximal reductions on day 28 of −84% and −91% change from baseline for the 2 mg/kg and 3 mg/kg monkey groups, respectively. To confirm the ELISA results, TTR protein was also quantitated by LC-MS, in which 4 unique TTR peptide fragments were quantitated in serum at each time point and the average of the 4 results is reported. LC-MS serum TTR quantitation, as illustrated in FIG. 22, confirmed that TTR was reduced at the first timepoint after infusion of the animals on day 7 and was maintained until necropsy on day 35. For the 2 mg/kg LNP3 dosed animals, maximal reduction of TTR protein was reached on day 35 (−82% change from baseline), while for the 3 mg/kg group maximal of TTR protein was reached on day 28 (−87% change from baseline).

Therefore, as described above and illustrated in the foregoing referenced figures, both the 2 mg/kg and 3 mg/kg LNP3 dosed NHPs resulted in marked relatively rapid liver TTR gene editing and corresponding reductions in serum TTR concentrations in protein.

### Safety Analysis

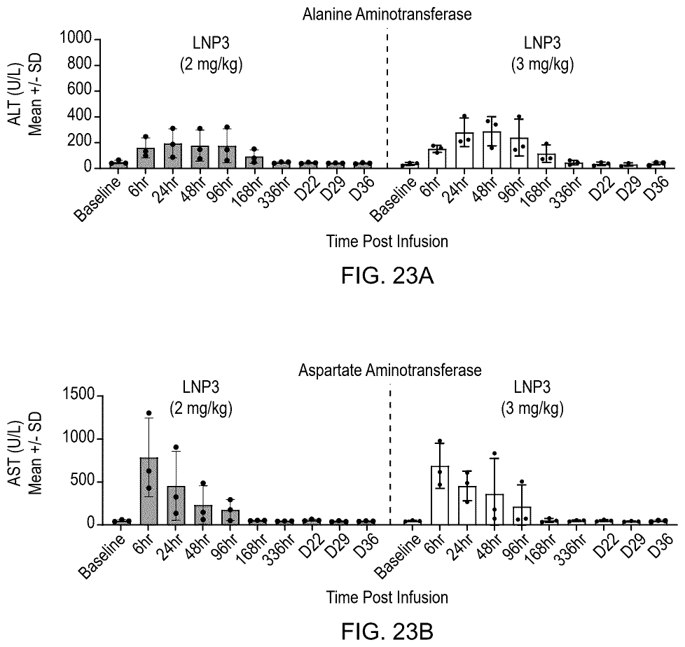

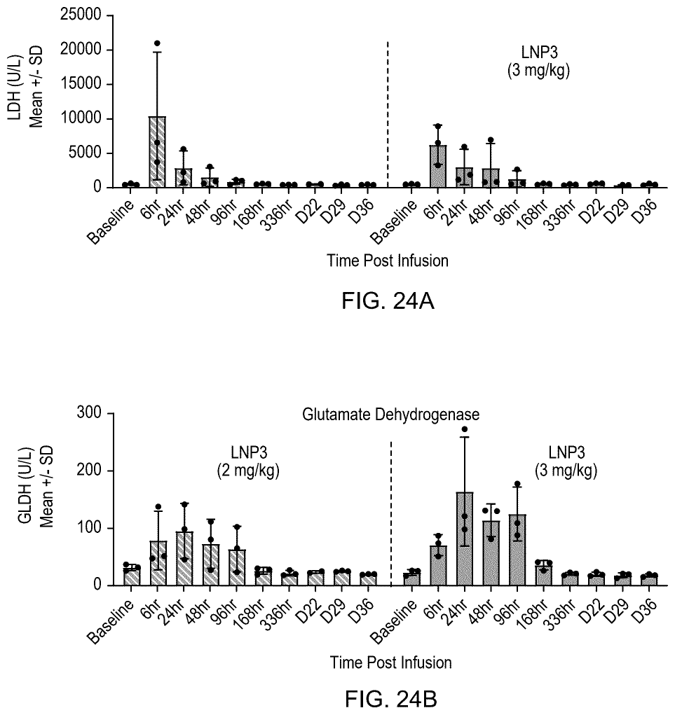

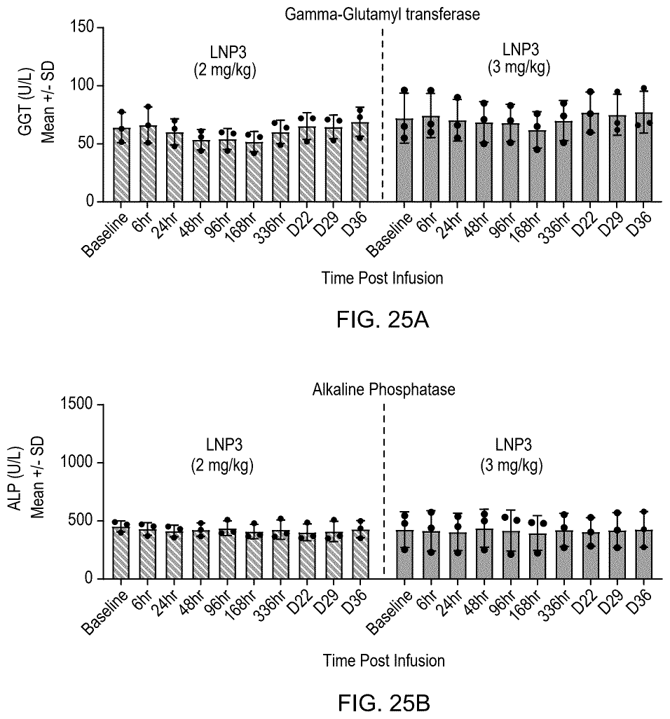

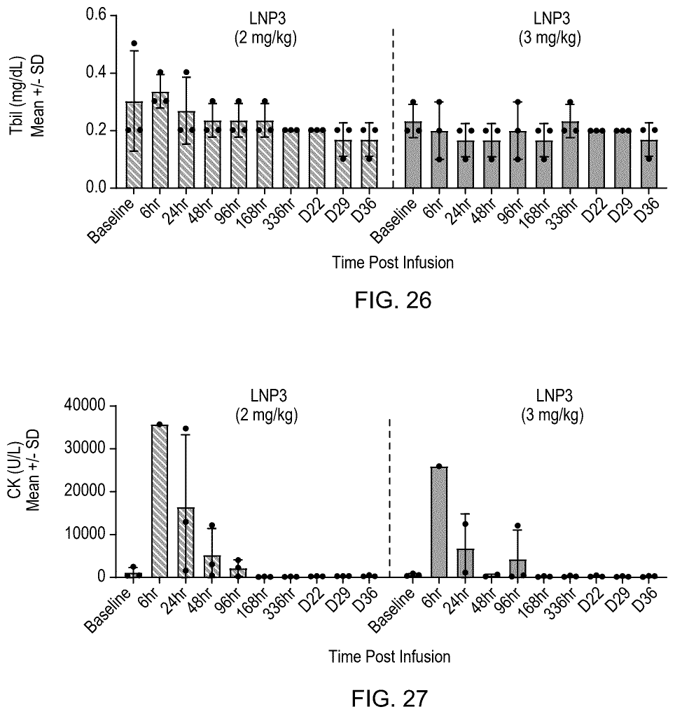

Blood serum was collected from each of the animals in the study at day-10, −7, −5 pre-infusion and 6, 24, 48, 96, 168, 336 hours, day 21, day 28, and day 35 post end of infusion for safety analysis and specifically directed at observing changes in liver enzymes and cytokine levels. Serum chemistry parameters were directly measured from blood serum samples on a Beckman Coulter AU680 analyzer. Values for day −10, −7, and −5 were averaged to obtain the baseline value. LNP3 dosed animals showed dose-dependent, transient alanine aminotransferase elevations as illustrated in FIG. 23A, which peaked at 24-48 hours post end of infusion and returned to baseline levels 336 hours post end of infusion. Aspartate aminotransferase levels, as illustrated in FIG. 23B, were elevated to a similar extent by both the 2 mg/kg and 3 mg/kg LNP3 doses, peaking at 6 hours post end of infusion and returning to baseline levels 168 hours post end of infusion. As illustrated in FIG. 24A, both the 2 mg/k and 3 mg/kg LNP3 doses elevated serum lactate dehydrogenase concentrations that returned to baseline levels by 168 hours post end of infusion. LNP3 also elevated glutamate dehydrogenase concentrations, as illustrated in FIG. 24B, in a dose-dependent manner, peaking at 24-hours, and returning to baseline levels 336 hours post end of infusion. Serum concentrations of gamma-glutamyl transferase and alkaline phosphatase, illustrated in FIGS. 25A and 25B, respectively, were not significantly changed by either LNP dose. In addition, LNP3 treatment did not significantly affect serum total bilirubin concentrations, as illustrated in FIG. 26. LNP3 elevated serum creatine kinase concentrations, as illustrated in FIG. 27, peaking at 6 hours post end of infusion then returning to baseline levels by 168 hours post end of infusion.

The analysis of the foregoing safety parameters in this aspect of the in vivo NHP study were consistent the prior aspect of the study in that they demonstrated that both doses of LNP3 produced a transient increase in liver enzymes that resolved rapidly within 2 weeks following dosing of the subjects.

### Pharmacokinetics (PK) Evaluation

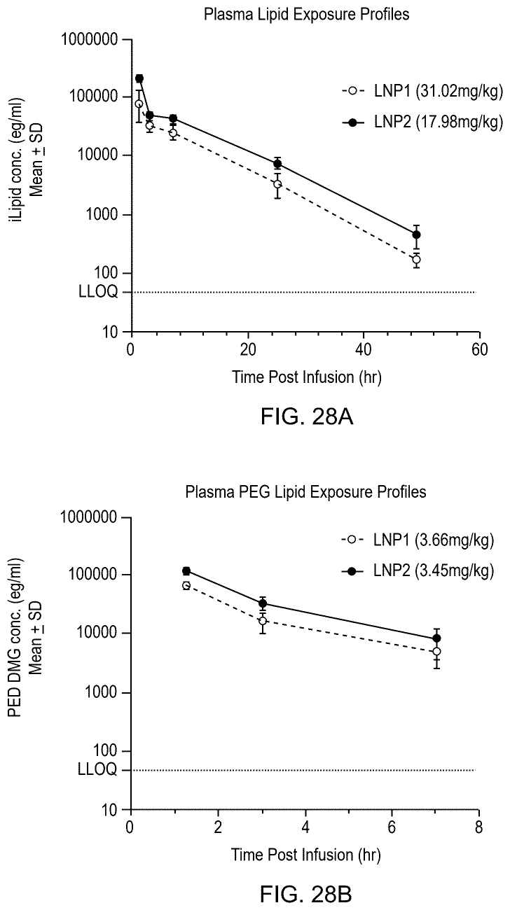

Blood samples were obtained from all animals (K₂EDTA) for plasma PK analysis and determination of concentrations of the ionizable amino lipid (iLipid) and PEGLipid that comprised LNP3. After the end of the infusion, plasma samples were collected at 0.25, 2, 6, 24, 48, 96, 168, 240, and 336 hours post LNP3 infusion. Concentrations of iLipid and PEG-Lipid were measured using qualified LC-MS assays. Dose-dependent iLipid plasma exposure was observed, as illustrated in FIG. 28A, declining below the LLOQ by 96 hours post end of infusion. Dose dependent plasma exposure of PEG lipid was also observed, as illustrated in FIG. 28B, reaching the LLOQ by 24 hours post end of infusion.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

### Example 4: TTR Gene Editing by GA521 Guide RNA

This example illustrates gene editing by an exemplary modified guide RNA, GA521.

Exemplary guide RNA GA521 was transfected into primary human hepatocytes using MessengerMax transfection. GA521 disrupts the start codon AUG of the TTR gene by editing it to ACG with an A-to-G base editor (e.g., ABE8.8; ABE8.8-m).

Three days after transfection, genomic DNA was harvested from the hepatocytes, and assessed for base editing with next-generation sequencing of PCR amplicons generated around the target splice site.

The human TTR locus primers used for NGS analysis are listed below:

With NGS adapters:
Forward (F):
(SEQ ID NO: 1192)
TCGTCGGCAGCGTCAGATGTGTATAAGAGACAGGATAAGCAGCCTAGCTC
AGGAGA
Reverse (R):
(SEQ ID NO: 1193)
GTCTCGTGGGCTCGGAGATGTGTATAAGAGACAGGGGCCAGCCTCAGACA
CAAA
Without NGS adapters:
Forward (F):
(SEQ ID NO: 950)
GATAAGCAGCCTAGCTCAGGAGA
Reverse (R):
(SEQ ID NO: 1194)
GGGCCAGCCTCAGACACAAA

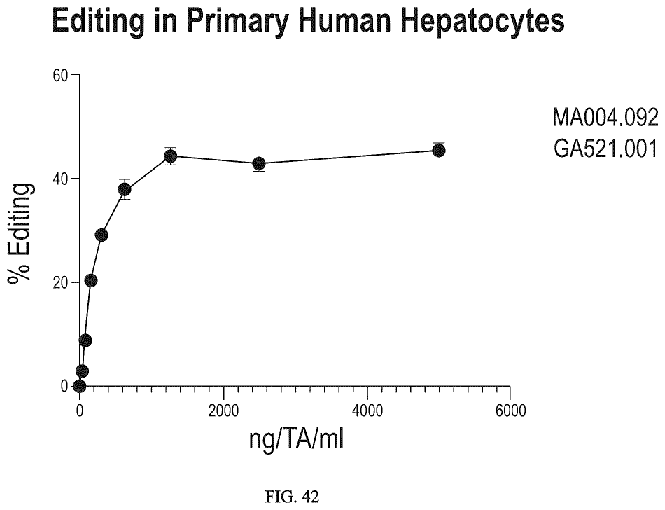

FIG. 42 depicts dose response for human gRNA GA521 in primary human hepatocytes. Percent base editing at various doses (ng/ml) total RNA was determined from NGS analysis. GA521 was the guide RNA.

Overall, GA521 showed an increase in base editing with increasing dose (ng/ml) total RNA and high and sustained editing activity of greater than 40% in human cells.

### Example 5: Transthyretin Gene Alterations

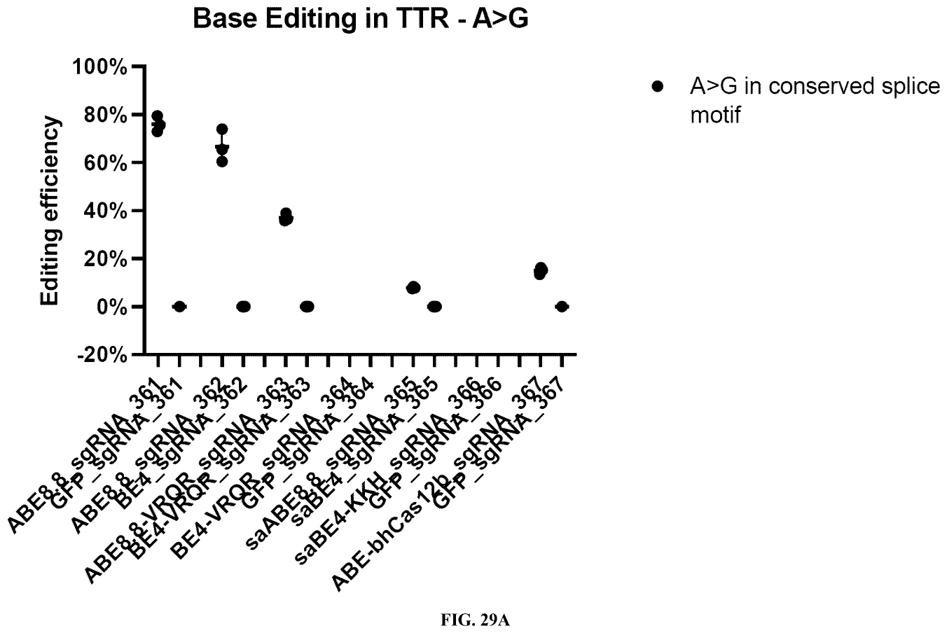

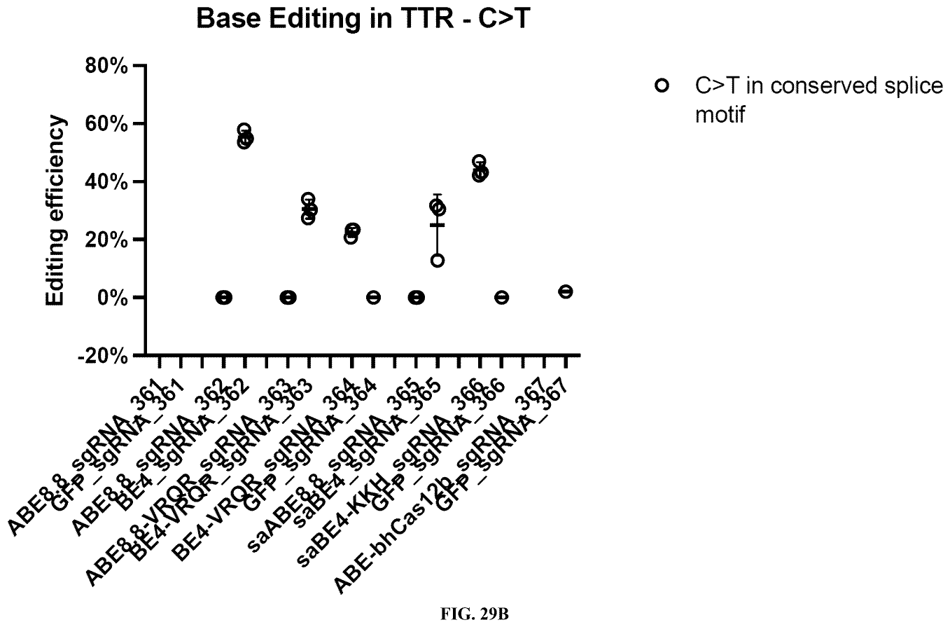

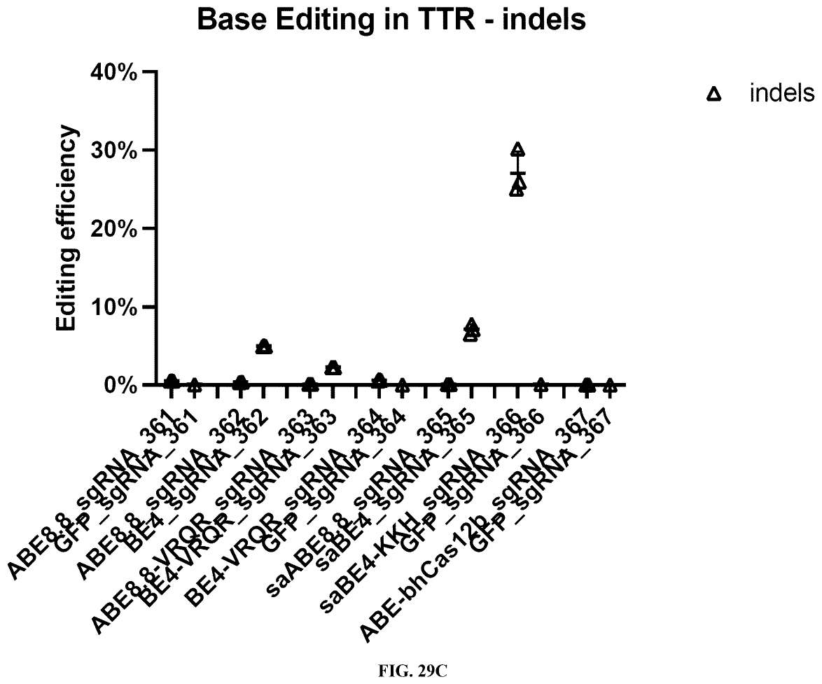

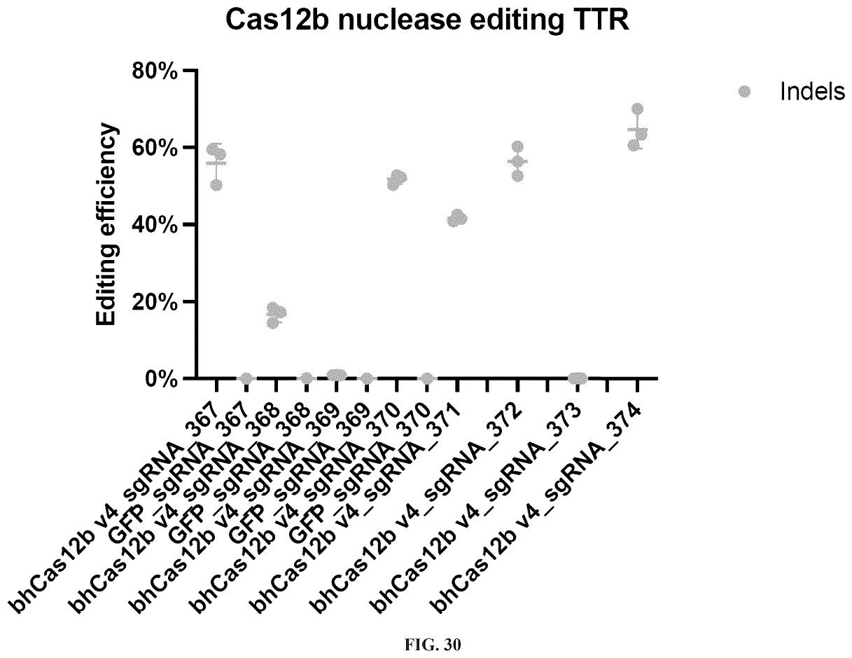

The guide RNAs listed in Table 1 were screened for use in editing the transthyretin (TTR) gene by disrupting splice sites (FIG. 29A-29C) or using a bhCas12b nuclease strategy (FIG. 30). 15 total guide RNAs were screened. The screen was performed in HEK293T cells using based editors and bhCas12b delivered as mRNA and the sgRNAs. The guide RNAs sgRNA_361 and sgRNA_362 worked well in splice site disruption (FIGS. 29A-29C) using ABE and/or BE4. Several of the gRNAs functioned well as bhCas 12b nuclease gRNAs.

Sequences for the base editors indicated in FIGS. 29A-29C and the bhCas12b endonuclease are listed below in Table 24.

TABLE 24
Base editor and nuclease sequences.

| | | SEQ | |
|---|---|---|---|
| | | ID | |
| Name | Description | NO | Sequence |
| ABE8.8 | pMRNA-trilink- | 1195 | MSEVEFSHEYWMRHALTLAKRARDEREVPVGAVLVL |
| | monoTadA- | | NNRVIGEGWNRAIGLHDPTAHAEIMALROGGLVMQN |
| | ABE8.8(TadA7. | | YRLIDATLYVTFEPCVMCAGAMIHSRIGRVVFGVRN |
| | 10+H123H+Y14 | | AKTGAAGSLMDVLHHPGMNHRVEITEGILADECAAL |
| | 7R+Q154R) | | LCRFFRMPRRVFNAQKKAQSSTDSGGSSGGSSGSET |
| | 120A bbsI | | PGTSESATPESSGGSSGGSDKKYSIGLAIGTNSVGW |
| | | | AVITDEYKVPSKKFKVLGNTDRHSIKKNLIGALLFD |
| | | | SGETAEATRLKRTARRRYTRRKNRICYLQEIFSNEM |
| | | | AKVDDSFFHRLEESFLVEEDKKHERHPIFGNIVDEV |
| | | | AYHEKYPTIYHLRKKLVDSTDKADLRLIYLALAHMI |
| | | | KFRGHFLIEGDLNPDNSDVDKLFIQLVQTYNQLFEE |
| | | | NPINASGVDAKAILSARLSKSRRLENLIAQLPGEKK |
| | | | NGLFGNLIALSLGLTPNFKSNFDLAEDAKLQLSKDT |
| | | | YDDDLDNLLAQIGDQYADLFLAAKNLSDAILLSDIL |
| | | | RVNTEITKAPLSASMIKRYDEHHQDLTLLKALVRQQ |
| | | | LPEKYKEIFFDQSKNGYAGYIDGGASQEEFYKFIKP |
| | | | ILEKMDGTEELLVKLNREDLLRKQRTFDNGSIPHQI |
| | | | HLGELHAILRRQEDFYPFLKDNREKIEKILTFRIPY |
| | | | YVGPLARGNSRFAWMTRKSEETITPWNFEEVVDKGA |
| | | | SAQSFIERMTNFDKNLPNEKVLPKHSLLYEYFTVYN |
| | | | ELTKVKYVTEGMRKPAFLSGEQKKAIVDLLFKTNRK |
| | | | VTVKQLKEDYFKKIECFDSVEISGVEDRFNASLGTY |
| | | | HDLLKIIKDKDFLDNEENEDILEDIVLTLTLFEDRE |
| | | | MIEERLKTYAHLFDDKVMKOLKRRRYTGWGRLSRKL |
| | | | INGIRDKQSGKTILDFLKSDGFANRNFMQLIHDDSL |
| | | | TFKEDIQKAQVSGQGDSLHEHIANLAGSPAIKKGIL |
| | | | QTVKVVDELVKVMGRHKPENIVIEMARENOTTQKGQ |
| | | | KNSRERMKRIEEGIKELGSQILKEHPVENTQLQNEK |
| | | | LYLYYLQNGRDMYVDQELDINRLSDYDVDHIVPQSF |
| | | | LKDDSIDNKVLTRSDKNRGKSDNVPSEEVVKKMKNY |
| | | | WRQLLNAKLITQRKFDNLTKAERGGLSELDKAGFIK |
| | | | RQLVETRQITKHVAQILDSRMNTKYDENDKLIREVK |
| | | | VITLKSKLVSDFRKDFQFYKVREINNYHHAHDAYLN |
| | | | AVVGTALIKKYPKLESEFVYGDYKVYDVRKMIAKSE |
| | | | QEIGKATAKYFFYSNIMNFFKTEITLANGEIRKRPL |
| | | | IETNGETGEIVWDKGRDFATVRKVLSMPQVNIVKKT |
| | | | EVQTGGFSKESILPKRNSDKLIARKKDWDPKKYGGF |
| | | | DSPTVAYSVLVVAKVEKGKSKKLKSVKELLGITIME |
| | | | RSSFEKNPIDFLEAKGYKEVKKDLIIKLPKYSLFEL |
| | | | ENGRKRMLASAGELQKGNELALPSKYVNFLYLASHY |
| | | | EKLKGSPEDNEQKOLFVEQHKHYLDEIIEQISEFSK |
| | | | RVILADANLDKVLSAYNKHRDKPIREQAENIIHLFT |
| | | | LTNLGAPAAFKYFDTTIDRKRYTSTKEVLDATLIHQ |
| | | | SITGLYETRIDLSQLGGDEGADKRTADGSEFESPKK |
| | | | KRKV |
| BE4 | pMRNA-BE4 | 1196 | MSSETGPVAVDPTLRRRIEPHEFEVFFDPRELRKET |
| | 120A BbsI | | CLLYEINWGGRHSIWRHTSQNTNKHVEVNFIEKFTT |
| | | | ERYFCPNTRCSITWFLSWSPCGECSRAITEFLSRYP |
| | | | HVTLFIYIARLYHHADPRNRQGLRDLISSGVTIQIM |
| | | | TEQESGYCWRNFVNYSPSNEAHWPRYPHLWVRLYVL |
| | | | ELYCIILGLPPCLNILRRKQPQLTFFTIALQSCHYQ |
| | | | RLPPHILWATGLKSGGSSGGSSGSETPGTSESATPE |
| | | | SSGGSSGGSDKKYSIGLAIGTNSVGWAVITDEYKVP |
| | | | SKKFKVLGNTDRHSIKKNLIGALLFDSGETAEATRL |
| | | | KRTARRRYTRRKNRICYLQEIFSNEMAKVDDSFFHR |
| | | | LEESFLVEEDKKHERHPIFGNIVDEVAYHEKYPTIY |
| | | | HLRKKLVDSTDKADLRLIYLALAHMIKFRGHFLIEG |
| | | | DLNPDNSDVDKLFIQLVQTYNQLFEENPINASGVDA |
| | | | KAILSARLSKSRRLENLIAQLPGEKKNGLFGNLIAL |
| | | | SLGLTPNFKSNFDLAEDAKLQLSKDTYDDDLDNLLA |
| | | | QIGDQYADLFLAAKNLSDAILLSDILRVNTEITKAP |
| | | | LSASMIKRYDEHHQDLTLLKALVROQLPEKYKEIFF |
| | | | DQSKNGYAGYIDGGASQEEFYKFIKPILEKMDGTEE |
| | | | LLVKLNREDLLRKQRTFDNGSIPHQIHLGELHAILR |
| | | | RQEDFYPFLKDNREKIEKILTFRIPYYVGPLARGNS |
| | | | RFAWMTRKSEETITPWNFEEVVDKGASAQSFIERMT |
| | | | NFDKNLPNEKVLPKHSLLYEYFTVYNELTKVKYVTE |
| | | | GMRKPAFLSGEQKKAIVDLLFKTNRKVTVKOLKEDY |
| | | | FKKIECFDSVEISGVEDRFNASLGTYHDLLKIIKDK |
| | | | DFLDNEENEDILEDIVLTLTLFEDREMIEERLKTYA |
| | | | HLFDDKVMKQLKRRRYTGWGRLSRKLINGIRDKQSG |
| | | | KTILDFLKSDGFANRNFMQLIHDDSLTFKEDIQKAQ |
| | | | VSGQGDSLHEHIANLAGSPAIKKGILQTVKVVDELV |
| | | | KVMGRHKPENIVIEMARENQTTQKGQKNSRERMKRI |
| | | | EEGIKELGSQILKEHPVENTQLQNEKLYLYYLONGR |
| | | | DMYVDQELDINRLSDYDVDHIVPQSFLKDDSIDNKV |
| | | | LTRSDKNRGKSDNVPSEEVVKKMKNYWRQLLNAKLI |
| | | | TQRKFDNLTKAERGGLSELDKAGFIKRQLVETRQIT |
| | | | KHVAQILDSRMNTKYDENDKLIREVKVITLKSKLVS |
| | | | DFRKDFQFYKVREINNYHHAHDAYLNAVVGTALIKK |
| | | | YPKLESEFVYGDYKVYDVRKMIAKSEQEIGKATAKY |
| | | | FFYSNIMNFFKTEITLANGEIRKRPLIETNGETGEI |
| | | | VWDKGRDFATVRKVLSMPQVNIVKKTEVQTGGFSKE |
| | | | SILPKRNSDKLIARKKDWDPKKYGGFDSPTVAYSVL |
| | | | VVAKVEKGKSKKLKSVKELLGITIMERSSFEKNPID |
| | | | FLEAKGYKEVKKDLIIKLPKYSLFELENGRKRMLAS |
| | | | AGELQKGNELALPSKYVNFLYLASHYEKLKGSPEDN |
| | | | EQKOLFVEQHKHYLDEIIEQISEFSKRVILADANLD |
| | | | KVLSAYNKHRDKPIREQAENIIHLFTLTNLGAPAAF |
| | | | KYFDTTIDRKRYTSTKEVLDATLIHQSITGLYETRI |
| | | | DLSQLGGDSGGSGGSGGSTNLSDIIEKETGKQLVIQ |
| | | | ESILMLPEEVEEVIGNKPESDILVHTAYDESTDENV |
| | | | MLLTSDAPEYKPWALVIQDSNGENKIKMLSGGSGGS |
| | | | GGSTNLSDIIEKETGKQLVIQESILMLPEEVEEVIG |
| | | | NKPESDILVHTAYDESTDENVMLLTSDAPEYKPWAL |
| | | | VIQDSNGENKIKMLSGGSKRTADGSEFESPKKKRKV |
| | | | E |
| ABE8.8- | pMRNA-trilink- | 1197 | MSEVEFSHEYWMRHALTLAKRARDEREVPVGAVLVL |
| VRQR | mono TadA- | | NNRVIGEGWNRAIGLHDPTAHAEIMALRQGGLVMQN |
| | ABE8.8(TadA7. | | YRLIDATLYVTFEPCVMCAGAMIHSRIGRVVFGVRN |
| | 10+H123H+Y14 | | AKTGAAGSLMDVLHHPGMNHRVEITEGILADECAAL |
| | 7R+Q154R)- | | LCRFFRMPRRVFNAQKKAQSSTDSGGSSGGSSGSET |
| | VRQR 120A | | PGTSESATPESSGGSSGGSDKKYSIGLAIGTNSVGW |
| | bbsI | | AVITDEYKVPSKKFKVLGNTDRHSIKKNLIGALLFD |
| | | | SGETAEATRLKRTARRRYTRRKNRICYLQEIFSNEM |
| | | | AKVDDSFFHRLEESFLVEEDKKHERHPIFGNIVDEV |
| | | | AYHEKYPTIYHLRKKLVDSTDKADLRLIYLALAHMI |
| | | | KFRGHFLIEGDLNPDNSDVDKLFIQLVQTYNQLFEE |
| | | | NPINASGVDAKAILSARLSKSRRLENLIAQLPGEKK |
| | | | NGLFGNLIALSLGLTPNFKSNFDLAEDAKLQLSKDT |
| | | | YDDDLDNLLAQIGDQYADLFLAAKNLSDAILLSDIL |
| | | | RVNTEITKAPLSASMIKRYDEHHQDLTLLKALVRQQ |
| | | | LPEKYKEIFFDQSKNGYAGYIDGGASQEEFYKFIKP |
| | | | ILEKMDGTEELLVKLNREDLLRKORTFDNGSIPHQI |
| | | | HLGELHAILRRQEDFYPFLKDNREKIEKILTFRIPY |
| | | | YVGPLARGNSRFAWMTRKSEETITPWNFEEVVDKGA |
| | | | SAQSFIERMTNFDKNLPNEKVLPKHSLLYEYFTVYN |
| | | | ELTKVKYVTEGMRKPAFLSGEQKKAIVDLLFKTNRK |
| | | | VTVKQLKEDYFKKIECFDSVEISGVEDRFNASLGTY |
| | | | HDLLKIIKDKDFLDNEENEDILEDIVLTLTLFEDRE |
| | | | MIEERLKTYAHLFDDKVMKOLKRRRYTGWGRLSRKL |
| | | | INGIRDKQSGKTILDFLKSDGFANRNFMQLIHDDSL |
| | | | TFKEDIQKAQVSGQGDSLHEHIANLAGSPAIKKGIL |
| | | | QTVKVVDELVKVMGRHKPENIVIEMARENOTTQKGQ |
| | | | KNSRERMKRIEEGIKELGSQILKEHPVENTQLQNEK |
| | | | LYLYYLQNGRDMYVDQELDINRLSDYDVDHIVPQSF |
| | | | LKDDSIDNKVLTRSDKNRGKSDNVPSEEVVKKMKNY |
| | | | WRQLLNAKLITQRKFDNLTKAERGGLSELDKAGFIK |
| | | | RQLVETRQITKHVAQILDSRMNTKYDENDKLIREVK |
| | | | VITLKSKLVSDFRKDFQFYKVREINNYHHAHDAYLN |
| | | | AVVGTALIKKYPKLESEFVYGDYKVYDVRKMIAKSE |
| | | | QEIGKATAKYFFYSNIMNFFKTEITLANGEIRKRPL |
| | | | IETNGETGEIVWDKGRDFATVRKVLSMPQVNIVKKT |
| | | | EVQTGGFSKESILPKRNSDKLIARKKDWDPKKYGGF |
| | | | VSPTVAYSVLVVAKVEKGKSKKLKSVKELLGITIME |
| | | | RSSFEKNPIDFLEAKGYKEVKKDLIIKLPKYSLFEL |
| | | | ENGRKRMLASARELQKGNELALPSKYVNFLYLASHY |
| | | | EKLKGSPEDNEQKOLFVEQHKHYLDEIIEQISEFSK |
| | | | RVILADANLDKVLSAYNKHRDKPIREQAENIIHLFT |
| | | | LTNLGAPAAFKYFDTTIDRKQYRSTKEVLDATLIHQ |
| | | | SITGLYETRIDLSQLGGDEGADKRTADGSEFESPKK |
| | | | KRKV |
| BE4- | pMRNA-BE4- | 1198 | MSSETGPVAVDPTLRRRIEPHEFEVFFDPRELRKET |
| VRQR | VRQR 120A | | CLLYEINWGGRHSIWRHTSQNTNKHVEVNFIEKFTT |
| | BsaI | | ERYFCPNTRCSITWFLSWSPCGECSRAITEFLSRYP |
| | | | HVTLFIYIARLYHHADPRNRQGLRDLISSGVTIQIM |
| | | | TEQESGYCWRNFVNYSPSNEAHWPRYPHLWVRLYVL |
| | | | ELYCIILGLPPCLNILRRKQPQLTFFTIALQSCHYQ |
| | | | RLPPHILWATGLKSGGSSGGSSGSETPGTSESATPE |
| | | | SSGGSSGGSDKKYSIGLAIGTNSVGWAVITDEYKVP |
| | | | SKKFKVLGNTDRHSIKKNLIGALLFDSGETAEATRL |
| | | | KRTARRRYTRRKNRICYLQEIFSNEMAKVDDSFFHR |
| | | | LEESFLVEEDKKHERHPIFGNIVDEVAYHEKYPTIY |
| | | | HLRKKLVDSTDKADLRLIYLALAHMIKFRGHFLIEG |
| | | | DLNPDNSDVDKLFIQLVQTYNQLFEENPINASGVDA |
| | | | KAILSARLSKSRRLENLIAQLPGEKKNGLFGNLIAL |
| | | | SLGLTPNFKSNFDLAEDAKLQLSKDTYDDDLDNLLA |
| | | | QIGDQYADLFLAAKNLSDAILLSDILRVNTEITKAP |
| | | | LSASMIKRYDEHHQDLTLLKALVRQQLPEKYKEIFF |
| | | | DQSKNGYAGYIDGGASQEEFYKFIKPILEKMDGTEE |
| | | | LLVKLNREDLLRKQRTFDNGSIPHQIHLGELHAILR |
| | | | RQEDFYPFLKDNREKIEKILTFRIPYYVGPLARGNS |
| | | | RFAWMTRKSEETITPWNFEEVVDKGASAQSFIERMT |
| | | | NFDKNLPNEKVLPKHSLLYEYFTVYNELTKVKYVTE |
| | | | GMRKPAFLSGEQKKAIVDLLFKTNRKVTVKOLKEDY |
| | | | FKKIECFDSVEISGVEDRFNASLGTYHDLLKIIKDK |
| | | | DFLDNEENEDILEDIVLTLTLFEDREMIEERLKTYA |
| | | | HLFDDKVMKQLKRRRYTGWGRLSRKLINGIRDKQSG |
| | | | KTILDFLKSDGFANRNFMQLIHDDSLTFKEDIQKAQ |
| | | | VSGQGDSLHEHIANLAGSPAIKKGILQTVKVVDELV |
| | | | KVMGRHKPENIVIEMARENQTTQKGQKNSRERMKRI |
| | | | EEGIKELGSQILKEHPVENTQLQNEKLYLYYLONGR |
| | | | DMYVDQELDINRLSDYDVDHIVPQSFLKDDSIDNKV |
| | | | LTRSDKNRGKSDNVPSEEVVKKMKNYWRQLLNAKLI |
| | | | TQRKFDNLTKAERGGLSELDKAGFIKRQLVETROIT |
| | | | KHVAQILDSRMNTKYDENDKLIREVKVITLKSKLVS |
| | | | DFRKDFQFYKVREINNYHHAHDAYLNAVVGTALIKK |
| | | | YPKLESEFVYGDYKVYDVRKMIAKSEQEIGKATAKY |
| | | | FFYSNIMNFFKTEITLANGEIRKRPLIETNGETGEI |
| | | | VWDKGRDFATVRKVLSMPQVNIVKKTEVQTGGFSKE |
| | | | SILPKRNSDKLIARKKDWDPKKYGGFVSPTVAYSVL |
| | | | VVAKVEKGKSKKLKSVKELLGITIMERSSFEKNPID |
| | | | FLEAKGYKEVKKDLIIKLPKYSLFELENGRKRMLAS |
| | | | ARELQKGNELALPSKYVNFLYLASHYEKLKGSPEDN |
| | | | EQKQLFVEQHKHYLDEIIEQISEFSKRVILADANLD |
| | | | KVLSAYNKHRDKPIREQAENIIHLFTLTNLGAPAAF |
| | | | KYFDTTIDRKQYRSTKEVLDATLIHQSITGLYETRI |
| | | | DLSQLGGDSGGSGGSGGSTNLSDIIEKETGKQLVIQ |
| | | | ESILMLPEEVEEVIGNKPESDILVHTAYDESTDENV |
| | | | MLLTSDAPEYKPWALVIQDSNGENKIKMLSGGSGGS |
| | | | GGSTNLSDIIEKETGKQLVIQESILMLPEEVEEVIG |
| | | | NKPESDILVHTAYDESTDENVMLLTSDAPEYKPWAL |
| | | | VIQDSNGENKIKMLSGGSKRTADGSEFESPKKKRKV |
| | | | E |
| saABE8.8 | pMRNA-trilink- | 1199 | MSEVEFSHEYWMRHALTLAKRARDEREVPVGAVLVL |
| | monoTadA- | | NNRVIGEGWNRAIGLHDPTAHAEIMALRQGGLVMQN |
| | saABE8.8 | | YRLIDATLYVTFEPCVMCAGAMIHSRIGRVVFGVRN |
| | (TadA7.10+H12 | | AKTGAAGSLMDVLHHPGMNHRVEITEGILADECAAL |
| | 3H+Y147R+Q1 | | LCRFFRMPRRVFNAQKKAQSSTDSGGSSGGSSGSET |
| | 54R) 120A bbsI | | PGTSESATPESSGGSSGGSKRNYILGLAIGITSVGY |
| | | | GIIDYETRDVIDAGVRLFKEANVENNEGRRSKRGAR |
| | | | RLKRRRRHRIQRVKKLLFDYNLLTDHSELSGINPYE |
| | | | ARVKGLSQKLSEEEFSAALLHLAKRRGVHNVNEVEE |
| | | | DTGNELSTKEQISRNSKALEEKYVAELQLERLKKDG |
| | | | EVRGSINRFKTSDYVKEAKQLLKVQKAYHQLDQSFI |
| | | | DTYIDLLETRRTYYEGPGEGSPFGWKDIKEWYEMLM |
| | | | GHCTYFPEELRSVKYAYNADLYNALNDLNNLVITRD |
| | | | ENEKLEYYEKFQIIENVFKQKKKPTLKQIAKEILVN |
| | | | EEDIKGYRVTSTGKPEFTNLKVYHDIKDITARKEII |
| | | | ENAELLDQIAKILTIYQSSEDIQEELTNLNSELTQE |
| | | | EIEQISNLKGYTGTHNLSLKAINLILDELWHTNDNQ |
| | | | IAIFNRLKLVPKKVDLSQQKEIPTTLVDDFILSPVV |
| | | | KRSFIQSIKVINAIIKKYGLPNDIIIELAREKNSKD |
| | | | AQKMINEMQKRNRQTNERIEEIIRTTGKENAKYLIE |
| | | | KIKLHDMQEGKCLYSLEAIPLEDLLNNPFNYEVDHI |
| | | | IPRSVSFDNSFNNKVLVKQEENSKKGNRTPFQYLSS |
| | | | SDSKISYETFKKHILNLAKGKGRISKTKKEYLLEER |
| | | | DINRFSVQKDFINRNLVDTRYATRGLMNLLRSYFRV |
| | | | NNLDVKVKSINGGFTSFLRRKWKFKKERNKGYKHHA |
| | | | EDALIIANADFIFKEWKKLDKAKKVMENQMFEEKQA |
| | | | ESMPEIETEQEYKEIFITPHQIKHIKDFKDYKYSHR |
| | | | VDKKPNRELINDTLYSTRKDDKGNTLIVNNLNGLYD |
| | | | KDNDKLKKLINKSPEKLLMYHHDPQTYQKLKLIMEQ |
| | | | YGDEKNPLYKYYEETGNYLTKYSKKDNGPVIKKIKY |
| | | | YGNKLNAHLDITDDYPNSRNKVVKLSLKPYRFDVYL |
| | | | DNGVYKFVTVKNLDVIKKENYYEVNSKCYEEAKKLK |
| | | | KISNQAEFIASFYNNDLIKINGELYRVIGVNNDLLN |
| | | | RIEVNMIDITYREYLENMNDKRPPRIIKTIASKTQS |
| | | | IKKYSTDILGNLYEVKSKKHPQIIKKGEGADKRTAD |
| | | | GSEFESPKKKRKV |
| saBE4 | pMRNA-saBE4 | 1200 | MSSETGPVAVDPTLRRRIEPHEFEVFFDPRELRKET |
| | 120A BbsI | | CLLYEINWGGRHSIWRHTSQNTNKHVEVNFIEKFTT |
| | | | ERYFCPNTRCSITWFLSWSPCGECSRAITEFLSRYP |
| | | | HVTLFIYIARLYHHADPRNRQGLRDLISSGVTIQIM |
| | | | TEQESGYCWRNFVNYSPSNEAHWPRYPHLWVRLYVL |
| | | | ELYCIILGLPPCLNILRRKQPQLTFFTIALQSCHYQ |
| | | | RLPPHILWATGLKSGGSSGGSSGSETPGTSESATPE |
| | | | SSGGSSGGSGKRNYILGLAIGITSVGYGIIDYETRD |
| | | | VIDAGVRLFKEANVENNEGRRSKRGARRLKRRRRHR |
| | | | IQRVKKLLFDYNLLTDHSELSGINPYEARVKGLSQK |
| | | | LSEEEFSAALLHLAKRRGVHNVNEVEEDTGNELSTK |
| | | | EQISRNSKALEEKYVAELQLERLKKDGEVRGSINRF |
| | | | KTSDYVKEAKQLLKVQKAYHQLDQSFIDTYIDLLET |
| | | | RRTYYEGPGEGSPFGWKDIKEWYEMLMGHCTYFPEE |
| | | | LRSVKYAYNADLYNALNDLNNLVITRDENEKLEYYE |
| | | | KFQIIENVFKQKKKPTLKQIAKEILVNEEDIKGYRV |
| | | | TSTGKPEFTNLKVYHDIKDITARKEIIENAELLDQI |
| | | | AKILTIYQSSEDIQEELTNLNSELTQEEIEQISNLK |
| | | | GYTGTHNLSLKAINLILDELWHTNDNQIAIFNRLKL |
| | | | VPKKVDLSQQKEIPTTLVDDFILSPVVKRSFIQSIK |
| | | | VINAIIKKYGLPNDIIIELAREKNSKDAQKMINEMQ |
| | | | KRNRQTNERIEEIIRTTGKENAKYLIEKIKLHDMQE |
| | | | GKCLYSLEAIPLEDLLNNPFNYEVDHIIPRSVSFDN |
| | | | SFNNKVLVKQEENSKKGNRTPFQYLSSSDSKISYET |
| | | | FKKHILNLAKGKGRISKTKKEYLLEERDINRFSVQK |
| | | | DFINRNLVDTRYATRGLMNLLRSYFRVNNLDVKVKS |
| | | | INGGFTSFLRRKWKFKKERNKGYKHHAEDALIIANA |
| | | | DFIFKEWKKLDKAKKVMENQMFEEKQAESMPEIETE |
| | | | QEYKEIFITPHQIKHIKDFKDYKYSHRVDKKPNREL |
| | | | INDTLYSTRKDDKGNTLIVNNLNGLYDKDNDKLKKL |
| | | | INKSPEKLLMYHHDPQTYQKLKLIMEQYGDEKNPLY |
| | | | KYYEETGNYLTKYSKKDNGPVIKKIKYYGNKLNAHL |
| | | | DITDDYPNSRNKVVKLSLKPYRFDVYLDNGVYKFVT |
| | | | VKNLDVIKKENYYEVNSKCYEEAKKLKKISNQAEFI |
| | | | ASFYNNDLIKINGELYRVIGVNNDLLNRIEVNMIDI |
| | | | TYREYLENMNDKRPPRIIKTIASKTQSIKKYSTDIL |
| | | | GNLYEVKSKKHPQIIKKGGSPKKKRKVSSDYKDHDG |
| | | | DYKDHDIDYKDDDDKSGGSGGSGGSTNLSDIIEKET |
| | | | GKQLVIQESILMLPEEVEEVIGNKPESDILVHTAYD |
| | | | ESTDENVMLLTSDAPEYKPWALVIQDSNGENKIKML |
| | | | SGGSGGSGGSTNLSDIIEKETGKQLVIQESILMLPE |
| | | | EVEEVIGNKPESDILVHTAYDESTDENVMLLTSDAP |
| | | | EYKPWALVIQDSNGENKIKMLSGGSKRTADGSEFES |
| | | | PKKKRKVE |
| saBE4- | pMRNA-saBE4- | 1201 | MSSETGPVAVDPTLRRRIEPHEFEVFFDPRELRKET |
| KKH | KKH 120A BbsI | | CLLYEINWGGRHSIWRHTSQNTNKHVEVNFIEKFTT |
| | | | ERYFCPNTRCSITWFLSWSPCGECSRAITEFLSRYP |
| | | | HVTLFIYIARLYHHADPRNRQGLRDLISSGVTIQIM |
| | | | TEQESGYCWRNFVNYSPSNEAHWPRYPHLWVRLYVL |
| | | | ELYCIILGLPPCLNILRRKQPQLTFFTIALQSCHYQ |
| | | | RLPPHILWATGLKSGGSSGGSSGSETPGTSESATPE |
| | | | SSGGSSGGSGKRNYILGLAIGITSVGYGIIDYETRD |
| | | | VIDAGVRLFKEANVENNEGRRSKRGARRLKRRRRHR |
| | | | IQRVKKLLFDYNLLTDHSELSGINPYEARVKGLSQK |
| | | | LSEEEFSAALLHLAKRRGVHNVNEVEEDTGNELSTK |
| | | | EQISRNSKALEEKYVAELQLERLKKDGEVRGSINRF |
| | | | KTSDYVKEAKQLLKVQKAYHQLDQSFIDTYIDLLET |
| | | | RRTYYEGPGEGSPFGWKDIKEWYEMLMGHCTYFPEE |
| | | | LRSVKYAYNADLYNALNDLNNLVITRDENEKLEYYE |
| | | | KFQIIENVFKQKKKPTLKQIAKEILVNEEDIKGYRV |
| | | | TSTGKPEFTNLKVYHDIKDITARKEIIENAELLDQI |
| | | | AKILTIYQSSEDIQEELTNLNSELTQEEIEQISNLK |
| | | | GYTGTHNLSLKAINLILDELWHTNDNQIAIFNRLKL |
| | | | VPKKVDLSQQKEIPTTLVDDFILSPVVKRSFIQSIK |
| | | | VINAIIKKYGLPNDIIIELAREKNSKDAQKMINEMQ |
| | | | KRNRQTNERIEEIIRTTGKENAKYLIEKIKLHDMQE |
| | | | GKCLYSLEAIPLEDLLNNPFNYEVDHIIPRSVSFDN |
| | | | SFNNKVLVKQEENSKKGNRTPFQYLSSSDSKISYET |
| | | | FKKHILNLAKGKGRISKTKKEYLLEERDINRFSVQK |
| | | | DFINRNLVDTRYATRGLMNLLRSYFRVNNLDVKVKS |
| | | | INGGFTSFLRRKWKFKKERNKGYKHHAEDALIIANA |
| | | | DFIFKEWKKLDKAKKVMENQMFEEKQAESMPEIETE |
| | | | QEYKEIFITPHQIKHIKDFKDYKYSHRVDKKPNRKL |
| | | | INDTLYSTRKDDKGNTLIVNNLNGLYDKDNDKLKKL |
| | | | INKSPEKLLMYHHDPQTYQKLKLIMEQYGDEKNPLY |
| | | | KYYEETGNYLTKYSKKDNGPVIKKIKYYGNKLNAHL |
| | | | DITDDYPNSRNKVVKLSLKPYRFDVYLDNGVYKFVT |
| | | | VKNLDVIKKENYYEVNSKCYEEAKKLKKISNQAEFI |
| | | | ASFYKNDLIKINGELYRVIGVNNDLLNRIEVNMIDI |
| | | | TYREYLENMNDKRPPHIIKTIASKTQSIKKYSTDIL |
| | | | GNLYEVKSKKHPQIIKKGGSPKKKRKVSSDYKDHDG |
| | | | DYKDHDIDYKDDDDKSGGSGGSGGSTNLSDIIEKET |
| | | | GKQLVIQESILMLPEEVEEVIGNKPESDILVHTAYD |
| | | | ESTDENVMLLTSDAPEYKPWALVIQDSNGENKIKML |
| | | | SGGSGGSGGSTNLSDIIEKETGKQLVIQESILMLPE |
| | | | EVEEVIGNKPESDILVHTAYDESTDENVMLLTSDAP |
| | | | EYKPWALVIQDSNGENKIKMLSGGSKRTADGSEFES |
| | | | PKKKRKVE |
| ABE- | pBZ517 | 1202 | MSEVEFSHEYWMRHALTLAKRARDEREVPVGAVLVL |
| bhCas12b | pCV021_ABE8. | | NNRVIGEGWNRAIGLHDPTAHAEIMALRQGGLVMQN |
| | 13m- | | YRLYDATLYVTFEPCVMCAGAMIHSRIGRVVFGVRN |
| | dBhCas12b_(D9 | | AKTGAAGSLMDVLHHPGMNHRVEITEGILADECAAL |
| | 52A, S893R,_K | | LCRFFRMPRRVFNAQKKAQSSTDGSSGSETPGTSES |
| | 846R, E837G) | | ATPESSGAPKKKRKVGIHGVPAAATRSFILKIEPNE |
| | | | EVKKGLWKTHEVLNHGIAYYMNILKLIRQEAIYEHH |
| | | | EQDPKNPKKVSKAEIQAELWDFVLKMQKCNSFTHEV |
| | | | DKDEVFNILRELYEELVPSSVEKKGEANQLSNKFLY |
| | | | PLVDPNSQSGKGTASSGRKPRWYNIKIAGDPSWEEE |
| | | | KKKWEEDKKKDPLAKILGKLAEYGLIPLFIPYTDSN |
| | | | EPIVKEIKWMEKSRNQSVRRLDKDMFIQALERFLSW |
| | | | ESWNLKVKEEYEKVEKEYKTLEERIKEDIQALKALE |
| | | | QYEKERQEQLLRDTLNTNEYRLSKRGLRGWREIIQK |
| | | | WLKMDENEPSEKYLEVFKDYQRKHPREAGDYSVYEF |
| | | | LSKKENHFIWRNHPEYPYLYATFCEIDKKKKDAKQQ |
| | | | ATFTLADPINHPLWVRFEERSGSNLNKYRILTEQLH |
| | | | TEKLKKKLTVQLDRLIYPTESGGWEEKGKVDIVLLP |
| | | | SRQFYNQIFLDIEEKGKHAFTYKDESIKFPLKGTLG |
| | | | GARVQFDRDHLRRYPHKVESGNVGRIYFNMTVNIEP |
| | | | TESPVSKSLKIHRDDFPKVVNFKPKELTEWIKDSKG |
| | | | KKLKSGIESLEIGLRVMSIALGQRQAAAASIFEVVD |
| | | | QKPDIEGKLFFPIKGTELYAVHRASFNIKLPGETLV |
| | | | KSREVLRKAREDNLKLMNQKLNFLRNVLHFQQFEDI |
| | | | TEREKRVTKWISRQENSDVPLVYQDELIQIRELMYK |
| | | | PYKDWVAFLKQLHKRLEVEIGKEVKHWRKSLSDGRK |
| | | | GLYGISLKNIDEIDRTRKFLLRWSLRPTEPGEVRRL |
| | | | EPGQRFAIDQLNHLNALKEDRLKKMANTIIMHALGY |
| | | | CYDVRKKKWQAKNPACQIILFEDLSNYNPYKERSRF |
| | | | ENSRLMKWSRREIPRQVALQGEIYGLQVGEVGAQFS |
| | | | SRFHAKTGSPGIRCRVVTKEKLQDNRFFKNLQREGR |
| | | | LTLDKIAVLKEGDLYPDKGGEKFISLSKDRKCVTTH |
| | | | ADINAAQNLQKRFWTRTHGFYKVYCKAYQVDGQTVY |
| | | | IPESKDQKQKIIEEFGEGYFILKDGVYEWVNAGKLK |
| | | | IKKGSSKQSSSELVDSDILKDSFDLASELKGEKLML |
| | | | YRDPSGNVFPSDKWMAAGVFFGKLERILISKLTNQY |
| | | | SISTIEDDSSKQSMKRPAATKKAGQAKKKK |
| bhCas12b | Cas12b v4 | 1203 | MAPKKKRKVGIHGVPAAATRSFILKIEPNEEVKKGL |
| v4 | | | WKTHEVLNHGIAYYMNILKLIRQEAIYEHHEQDPKN |
| | | | PKKVSKAEIQAELWDFVLKMQKCNSFTHEVDKDEVF |
| | | | NILRELYEELVPSSVEKKGEANQLSNKFLYPLVDPN |
| | | | SQSGKGTASSGRKPRWYNLKIAGDPSWEEEKKKWEE |
| | | | DKKKDPLAKILGKLAEYGLIPLFIPYTDSNEPIVKE |
| | | | IKWMEKSRNQSVRRLDKDMFIQALERFLSWESWNLK |
| | | | VKEEYEKVEKEYKTLEERIKEDIQALKALEQYEKER |
| | | | QEQLLRDTLNTNEYRLSKRGLRGWREIIQKWLKMDE |
| | | | NEPSEKYLEVFKDYQRKHPREAGDYSVYEFLSKKEN |
| | | | HFIWRNHPEYPYLYATFCEIDKKKKDAKQQATFTLA |
| | | | DPINHPLWVRFEERSGSNLNKYRILTEQLHTEKLKK |
| | | | KLTVQLDRLIYPTESGGWEEKGKVDIVLLPSRQFYN |
| | | | QIFLDIEEKGKHAFTYKDESIKFPLKGTLGGARVQF |
| | | | DRDHLRRYPHKVESGNVGRIYFNMTVNIEPTESPVS |
| | | | KSLKIHRDDFPKVVNFKPKELTEWIKDSKGKKLKSG |
| | | | IESLEIGLRVMSIDLGQRQAAAASIFEVVDQKPDIE |
| | | | GKLFFPIKGTELYAVHRASFNIKLPGETLVKSREVL |
| | | | RKAREDNLKLMNQKLNFLRNVLHFQQFEDITEREKR |
| | | | VTKWISRQENSDVPLVYQDELIQIRELMYKPYKDWV |
| | | | AFLKQLHKRLEVEIGKEVKHWRKSLSDGRKGLYGIS |
| | | | LKNIDEIDRTRKFLLRWSLRPTEPGEVRRLEPGQRF |
| | | | AIDQLNHLNALKEDRLKKMANTIIMHALGYCYDVRK |
| | | | KKWQAKNPACQIILFEDLSNYNPYGERSRFENSRLM |
| | | | KWSRREIPRQVALQGEIYGLQVGEVGAQFSSRFHAK |
| | | | TGSPGIRCRVVTKEKLQDNRFFKNLQREGRLTLDKI |
| | | | AVLKEGDLYPDKGGEKFISLSKDRKCVTTHADINAA |
| | | | QNLQKRFWTRTHGFYKVYCKAYQVDGQTVYIPESKD |
| | | | QKQKIIEEFGEGYFILKDGVYEWVNAGKLKIKKGSS |
| | | | KQSSSELVDSDILKDSFDLASELKGEKLMLYRDPSG |
| | | | NVFPSDKWMAAGVFFGKLERILISKLTNQYSISTIE |
| | | | DDSSKQSMSGGSKRTADGSEFESPKKKRKVE |

### Example 6: Confirmation of Loss of Transthyretin (TTR) Expression in Hepatocytes

The guide RNAs identified in Example 1 as working well in splice site disruption using ABE and/or BE4, or as working well with bhCas12b are used to edit transthyretin (TTR) in hepatocytes to result in loss or reduction in TTR expression. Standard methods for culturing hepatocytes are used (see, e.g., Shulman and Nahmias, “Long-term and coculture of primary rate and human hepatocytes”, Methods Mol. Biol., 945:287-302 (2013); and Castell J., Gómez-Lechón M. (2009) Liver Cell Culture Techniques. In: Dhawan A., Hughes R. (eds) Hepatocyte Transplantation. Methods in Molecular Biology, vol 481. Humana Press, Totowa, NJ. doi: 10.1007/978-1-59745-201-4_4). For gene editing, the base editors and bhCas12b are delivered to the cells as mRNA in combination with sgRNA using lipid nanoparticles. Following gene editing, transthyretin (TTR) expression in the cells is confirmed as reduced or eliminated. Reduction or elimination of expression is confirmed using standard techniques in molecular biology (e.g., Real-Time Quantitative Reverse Transcription PCR).

### Example 7: Direct Correction of the Transthyretin (TTR) V122I Mutation

The mutation V1221 in the mature transthyretin (TTR) polypeptide is the African American population founder mutation. The mutation is a major cause of cardiovascular mortality (i.e., cardiac amyloidosis) for the African American population. About 3.9% of African Americans have the V122I mutation. The V122I mutation can be edited using ABE. Thus, ABE is used to directly correct the V122I mutation in cells.

ABE mRNA and sgRNA are delivered to a cell (e.g., a hepatocyte or a HEK293T cell) encoding a transthyretin (TTR) polypeptide having the V122I mutation. ABE mRNA encoding the base editors indicated in Table 25 below are administered in combination with sgRNAs comprising the indicated spacer sequences. The transthyretin (TTR) gene in the cell is successfully edited to no longer encode the pathogenic V122I mutation and to encode a non-pathogenic version of transthyretin (e.g., transthyretin with a valine at position 122).

TABLE 25
Base editor and nuclease sequences. One of skill in the art will understand
that some of the target site sequences correspond to a reverse-complement to the above-
provided transthyretin polynucleotide sequence; i.e., the target sequences may
correspond to either strand of a dsDNA molecule encoding a transthyretin
polynucleotide.

| BASE | | | | | TARGET |
|---|---|---|---|---|---|
| EDITOR | SGRNA | SPACER SEQUENCE | PAM | TARGET SEQUENCE | BASE |
| SPCAS9- | SGRNA_ | GGCUAUCGUCACCAAUC | AGG | GGCTATCGTCACCAATC | 5A |
| ABE | 375 | CCA (SEQ ID NO: 637) | | CCA (SEQ ID NO: 651) | |
| SPCAS9- | SGRNA_ | GCUAUCGUCACCAAUCC | GGA | GCTATCGTCACCAATCC | 4A |
| VRQR- | 376 | CAA (SEQ ID NO: 638) | | CAA (SEQ ID NO: 652) | |
| ABE | | | | | |
| SACAS9- | SGRNA_ | GGCUAUCGUCACCAAUC | AGGAA | GGCTATCGTCACCAATC | 5A |
| ABE | 377 | CCA (SEQ ID NO: 637) | T | CCA (SEQ ID NO: 651) | |

In embodiments, the altered amino acid is in a splice site or start codon as illustrated in the following sequences. Alterations in splice site disrupt expression of the encoded TTR polypeptide. A description of the respective target for each of the following sequences is indicated in parentheses:

4A of the nucleotide sequence
(SEQ ID NO: 469)
TATAGGAAAACCAGTGAGTC (splice sites);
6A of the nucleotide sequence
(SEQ ID NO: 470)
TACTCACCTCTGCATGCTCA (splice sites);
5A of the nucleotide sequence
(SEQ ID NO: 639)
ACTCACCTCTGCATGCTCAT (splice sites);
7A of the nucleotide sequence
(SEQ ID NO: 641)
ATACTCACCTCTGCATGCTCA (splice sites);
6A of the nucleotide sequence
(SEQ ID NO: 643)
TTGGCAGGATGGCTTCTCATCG (splice sites);
9A of the sequence
(SEQ ID NO: 643)
TTGGCAGGATGGCTTCTCATCG (start codon);
5A of the sequence
(SEQ ID NO: 651)
GGCTATCGTCACCAATCCCA (correction of pathogenic
mutation);
4A of the sequence
(SEQ ID NO: 652)
GCTATCGTCACCAATCCCAA (correction of pathogenic
mutation);
7C of the nucleotide sequence
(SEQ ID NO: 470)
TACTCACCTCTGCATGCTCA (splice sites);
6C of the nucleotide sequence
(SEQ ID NO: 639)
ACTCACCTCTGCATGCTCAT (splice sites);
7C of the nucleotide sequence
(SEQ ID NO: 640)
TACCACCTATGAGAGAAGAC (splice sites);
8C of the nucleotide sequence
(SEQ ID NO: 641)
ATACTCACCTCTGCATGCTCA (splice sites);
or
11C of the nucleotide sequence
(SEQ ID NO: 642)
ACTGGTTTTCCTATAAGGTGT (splice sites).

### Example 8: Transthyretin (TTR) Guide Screening and Functional Knockdown Assessment in Primary Hepatocytes

Experiments were undertaken to determine the efficacy of the base editor systems developed in the above Examples in editing human or primate primary hepatocytes. As described above, fifteen guide RNAs were designed to knockdown transthyretin (TTR) protein expression in HEK293T cells. These guides used either a base editing strategy for splice site disruption or a nuclease-based bhCas 12b strategy. A base editing strategy was initially prioritized. Base editing guides were used with either an ABE (adenosine base editor) or CBE (cytidine base editor) for splice site disruption, and a subset of guides was suitable for use with both an ABE and a CBE. Six guide editor combinations exhibited good editing efficiency in HEK293T cells (FIGS. 29A-29C): ABE8.8_sgRNA_361; ABE8.8_sgRNA_362; BE4_sgRNA_362; ABE8.8-VRQR_sgRNA 363; BE4-VRQR_sgRNA_363; and BE4-KKH_sgRNA_366. Experiments were undertaken to evaluate these four guides (sgRNAs 361, 362, 363, 366; sequences are listed in Table 1) in primary hepatocytes (both human and Macaca fascicularis) to assess editing efficiency in primary cells and the capacity for functional knockdown of TTR protein expression.

### Screening Hek293T-Validated TTR Knockdown Guides in PXB-Cell Primary Human Hepatocytes

Editor mRNA sgRNA combinations (i.e. base editor systems) were transfected in triplicate in human hepatocytes extracted from humanized mouse livers (PXB-cells, PhoenixBio) following a 3-day cell incubation. In addition to the 6 guide-editor pairs of interest (ABE8.8_sgRNA_361; ABE8.8_sgRNA_362; BE4_sgRNA_362; ABE8.8-VRQR_sgRNA_363; BE4-VRQR_sgRNA_363; and BE4-KKH_sgRNA_366), two positive control guide-editor pairs were also transfected. These positive controls included ABE8.8_sgRNA_088, which conained the spacer sequence CAGGAUCCGCACAGACUCCA

(SEQ ID NO: 1204) and is known to be effective at editing sites outside of the TTR gene, and Cas9_gRNA991 (Gillmore, J. D. et al. “CRISPR-Cas9 In Vivo Gene Editing for Transthyretin Amyloidosis,” New Engl J Med 385, 493-502 (2021)), which contained the spacer sequence AAAGGCUGCUGAUGACACCU (SEQ ID NO: 1205) corresponding to the target sequence AAAGGCTGCTGATGACACCT (SEQ ID NO: 1206). The guide gRNA991 is known to be effective for use in inducing functional TTR knockdown in hepatocytes. An untreated condition was also included as a negative control. To assess functional TTR knockdown, cell supernatants were collected and stored at −80° C. Collections were performed prior to transfection (3-day incubation), as well as 4—, 7—, 10—, and 13-days post-transfection. An additional media change was performed 1 day post-transfection, but the supernatant was discarded. Genomic DNA was harvested from cells 13 days post-transfection and editing efficiency was assessed by Next Generation Sequencing (NGS). A human TTR ELISA assay was used to assess TTR protein concentration in cell supernatants pre-transfection, as well as 7-days and 13-days post-transfection.

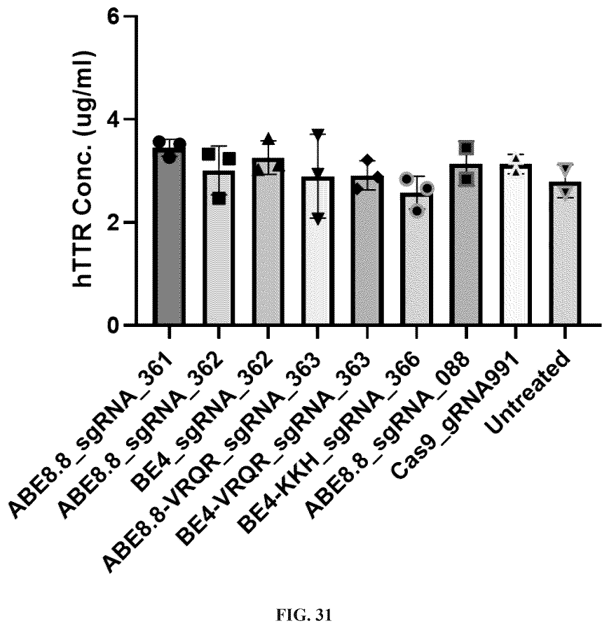

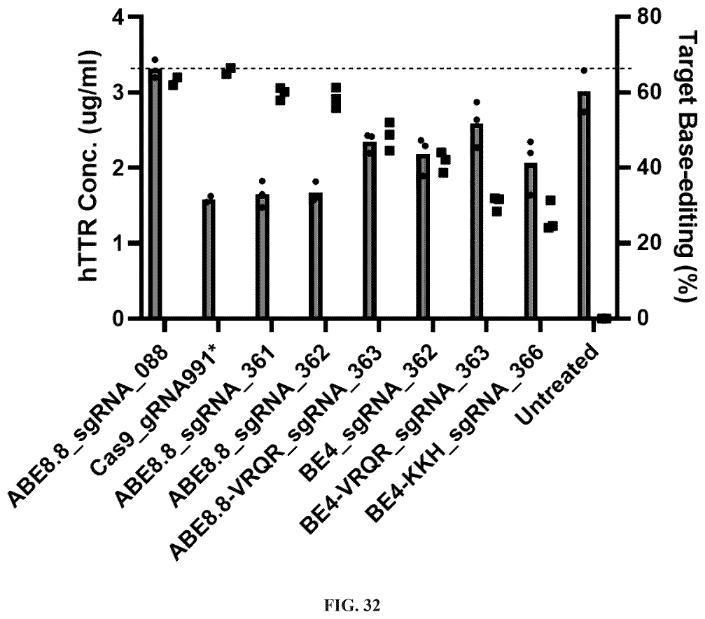

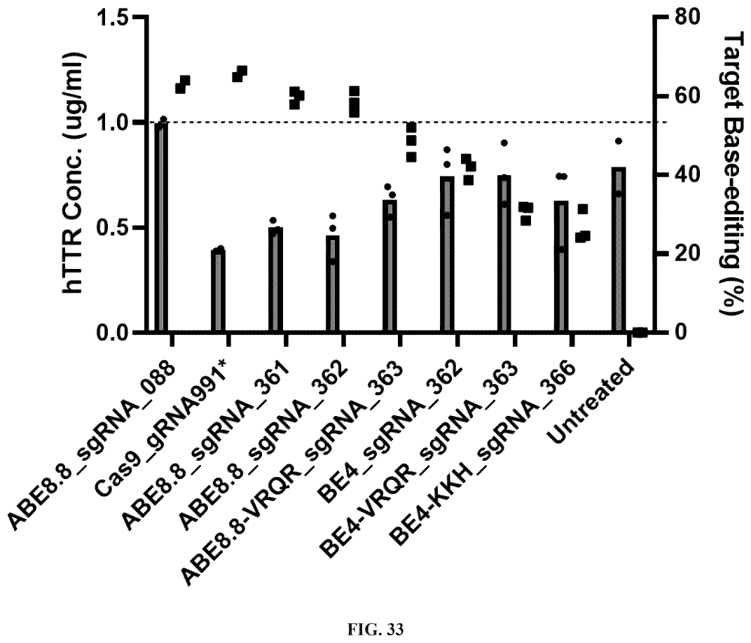

Pre-transfection, no significant difference in TTR concentration was observed between samples (FIG. 31). By 7-days post-transfection, a roughly 50% reduction in TTR levels was observed for ABE8.8_sgRNA_361 and ABE8.8_sgRNA_362 as compared to the control ABE8.8_sgRNA_088, which did not edit within the TTR gene (FIG. 32). This reduction was comparable to the positive control Cas9_gRNA991 (FIG. 32). Similar trends were observed 13-days post-transfection (FIG. 33). Editing efficiencies for ABE8.8_sgRNA_361 and ABE8.8_sgRNA_362 were both high, at approximately 60% (FIGS. 30 and 33). This was comparable to the controls ABE8.8_sgRNA_088 and Cas9_gRNA991 (FIGS. 32 and 33). TTR protein knockdown was positively correlated with editing rates across samples (FIGS. 32 and 33).

Assessing Editing Performance and Functional Knockdown Generation for ABE8.8_sgRNA361 and ABE8.8_sgRNA362 in Primary Cyno Hepatocytes

ABE8.8_sgRNA_361 and ABE8.8_sgRNA_362, both of which exhibited high target base-editing and functional TTR protein knockdown in PXB-cells, were transfected in triplicate in primary cyno (Macaca fascicularis) hepatocyte co-cultures. ABE8.8_sgRNA_088 was transfected as a positive control, and an untreated condition was included as a negative control, both in triplicate. To assess functional TTR knockdown, cell supernatants were collected and stored at −80° C. Collections were performed prior to transfection (3-day incubation), as well as 4—, 7—, 10—, and 13-days post-transfection. An additional media change was performed 1 day post-transfection, but the supernatant was discarded. Genomic DNA was harvested from cells 13 days post-transfection and editing efficiency was assessed by Next Generation Sequencing (NGS). A modified TTR ELISA assay was used to assess cyno TTR protein concentration in cell supernatants pre-transfection, as well as 7-days and 13-days post-transfection.

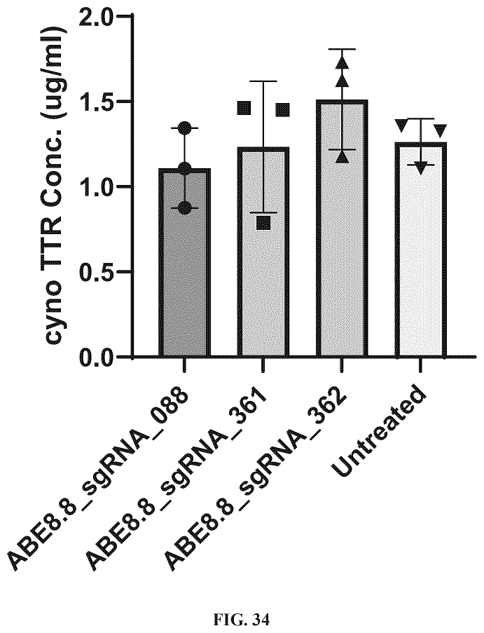

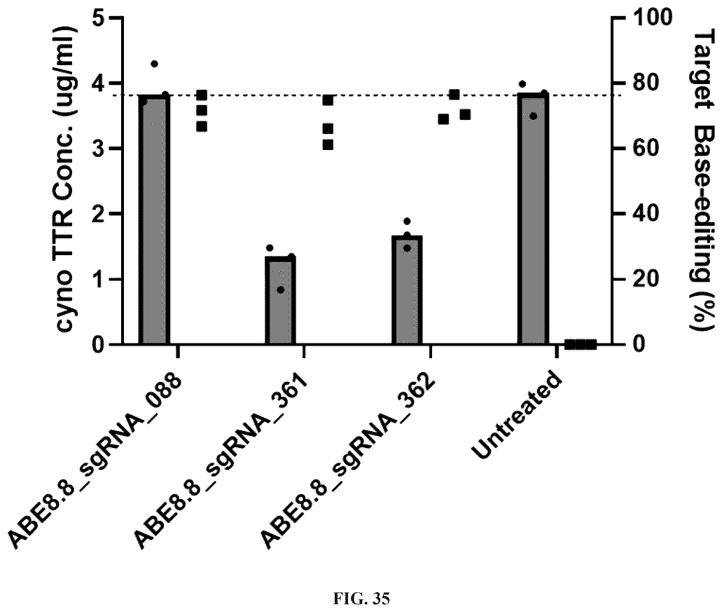

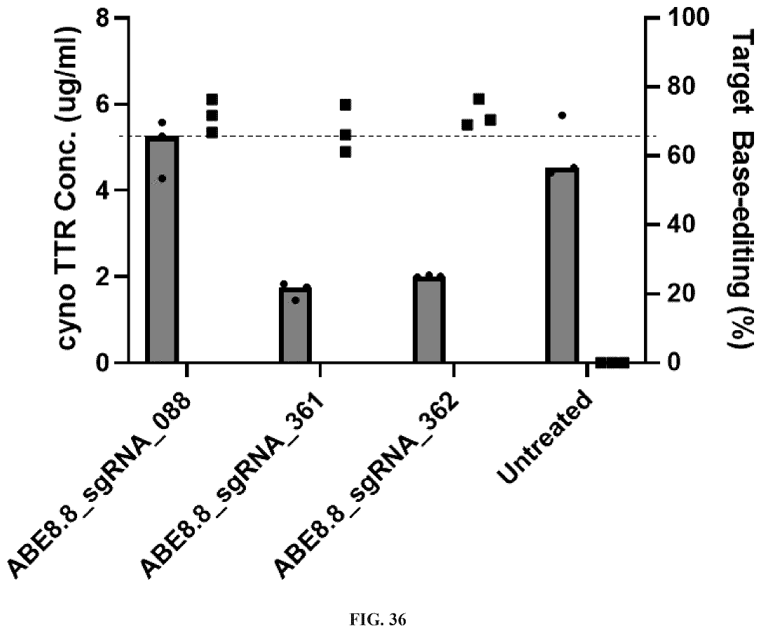

Pre-transfection, no significant difference in cyno TTR concentration was observed between samples (FIG. 34). By 7-days post-transfection, roughly 60-70% reductions in cyno TTR levels were observed for ABE8.8_sgRNA_361 and ABE8.8_sgRNA_362 as compared to ABE8.8_sgRNA_088, which did not edit within the TTR gene (FIG. 35). Similar trends were observed 13-days post-transfection (FIG. 36). Editing efficiencies for ABE8.8_sgRNA_361 and ABE8.8_sgRNA_362 were both high, at approximately 70% (FIGS. 35 and 36). This was comparable to the ABE8.8_sgRNA_088 positive control (FIGS. 35 and 36).

The following materials and methods were employed in this Example.

### Pxb-Cell Maintenance

One 24-well plate of PXB-cell hepatocytes was ordered from PhoenixBio. After receipt of cells, media was changed twice with pre-warmed dHCGM media (PhoenixBio)+10% Fetal Bovine Serum (Thermo Fisher, A³¹⁶⁰⁴⁰¹). Cells were then incubated according to the manufacturer's instructions, changing the media every 3 days. An extra media change was performed the day following transfection, after which a 3-day media change schedule was resumed. For all media changes other than the two initial changes and the day following transfection (pre-transfection and 4, 7, 10, and 13 days post-transfection), media was collected, distributed across multiple 96-well plates, and stored at −80° C.

### Primary Cyno Hepatocyte (PCH) Co-Culture Generation and Maintenance

A frozen vial of primary cyno hepatocytes (IVAL, A⁷⁵²⁴⁵, Lot #10286011) was thawed and mixed with 50 mL pre-warmed CHRM medium (Invitrogen, CM7000). Tube was centrifuged at 100×g for 10 minutes at room temperature. CHRM media was discarded and cell pellet was resuspended in 4 mL INVITROGRO CP Medium (Bio IVT, Z990003)+2.2% Torpedo Antibiotic Mix (Bio IVT, Z99000). Cells were counted using a Neubauer Improved hemocytometer (SKC, Inc., DHCN015) and 350,000 cells/well were plated in a 24-well BioCoat Rat Collagen I plate (Corning, 354408). There was a sufficient number of cells for 18 wells. Co-cultures were generated 5 hours after plating through the addition of 20,000 3T3-J2 cells (Stem Cell Technologies, 100-0353) in fresh CP+Torpedo media to each well. Following a media change the next day, cells were incubated according to the manufacturer's instructions, changing CP+Torpedo media every 3 days. An extra media change was performed the day following transfection, after which a 3-day media change schedule was resumed.

### Cell Transfection

PXB-cells were transfected 3 days following their receipt. Prior to transfection, a media change was performed for all wells. Spent media was aliquoted across multiple 96 well plates and stored at −80° C. For each condition, 200 ng sgRNA (Agilent and Synthego) and 600 ng editor mRNA (produced at Beam) were diluted to 25 μl with OPTIMEM (Thermo Fisher, 31985062) in a 96-well plate. Separately, the transfection reagent lipofectamine MessengerMAX Reagent (Thermo Fisher, LMRNA015) at 1.5X the total volume of RNA was diluted in the reduced-serum medium OPTIMEM to 25 μl for each condition, mixed thoroughly, and incubated at room temperature for 10 minutes. MessengerMAX solutions were then combined with the corresponding sgRNA+editor solution and thoroughly mixed. Following a 5-minute incubation at room temperature, the lipid encapsulated mRNA+sgRNA mixes were added dropwise onto the PXB-cells. Media was changed and spent media was discarded <16 hours following transfection.

PCH samples were transfected 4 days following the addition of 3T3-J2 feeder cells. Prior to transfection, a media change was performed for all wells. The same transfection protocol as that used for PXB-cells was used for PCH.

### Next Generation DNA Sequencing (NGS)

Following media collection, genomic DNA was isolated from each PXB-cell well 13-days post-transfection according to the following protocol. 200 μl of QuickExtract DNA Extraction Solution (Lucigen, QE09050) was added to each well. Cells were incubated for 5 minutes at 37° C., after which the cells were manually dislodged from the bottom of each well by pipetting. The cells were incubated again for 5 minutes at 37° C., after which the buffer-cell mixture was thoroughly mixed, and 150 μl was transferred to a 96-well plate. The 96-well plate was incubated at 65° C. for 15 mins and then at 98° C. for 10 mins.

PCR was performed using Phusion U Green Multiplex PCR Master Mix (Fisher Scientific, F564L) and region-specific primers. A second round of PCR was then performed on the first round PCR products to add barcoded Illumina adaptor sequences to each sample. Second round PCR products were purified using SPRIselect beads (Thermo Fisher Scientific, B23317) at a 1:1 bead to PCR ratio. The combined library concentration was quantified using a Qubit 1X dsDNA HS Assay Kit (Thermo Fisher Scientific, Q33231), and the library was sequenced using a Miseq Reagent Micro Kit v2 (300-cycles) (Illumina, MS-103-1002). Reads were aligned to appropriate reference sequences and editing efficiency was assessed at the appropriate sites.

Genomic DNA isolation, NGS, and analysis were performed as above for PCH. The library was sequenced using a Miseq Reagent Nano Kit v2 (300-cycles) (Illumina, MS-103-1001).

### Ttr Protein Quantification

A human prealbumin (TTR) ELISA kit (Abcam, ab231920) was used to measure TTR protein levels in PXB-cell supernatants at various timepoints pre- and post-transfection. PXB-cell supernatants were thawed at room temperature and centrifuged at 2000×g for 10 minutes at 4° C. Supernatants were then diluted 1:1000 in provided Sample Diluent NS buffer prior to loading on the ELISA plate. The ELISA assay was then performed according to manufacturer's instructions. Samples were allowed to develop for 18 minutes in Development solution prior to addition of Stop solution. Absorbance was read at 450 nm using an Infinite M Plex plate reader (Tecan).

For the detection of cyno (Macaca fascicularis) TTR protein in primary cyno hepatocyte co-culture supernatants, known concentrations of purified cyno TTR protein (Abcam, ab239566) were used to assess cross reactivity of the human TTR ELISA kit (Abcam, ab231920). Through this approach, it was determined that the kit was approximately 4% cross-reactive with cyno TTR protein. Purified cyno TTR protein was then used to generate a new set of standards (20 ng-0.3125 ng for standards 1-7) capable of accurately measuring cyno TTR protein levels. The assay was otherwise performed identically to manufacturer's instructions. Supernatants were diluted 1:1000 and were developed for 17 minutes in Development solution prior to addition of Stop solution.

### Example 9: Transthyretin (TTR) Promoter Screening for Gene Expression Knockdown

Experiments were undertaken to develop base editor systems suitable for knocking out expression of the TTR gene in humans through introducing alterations to the promoter region of the gene.

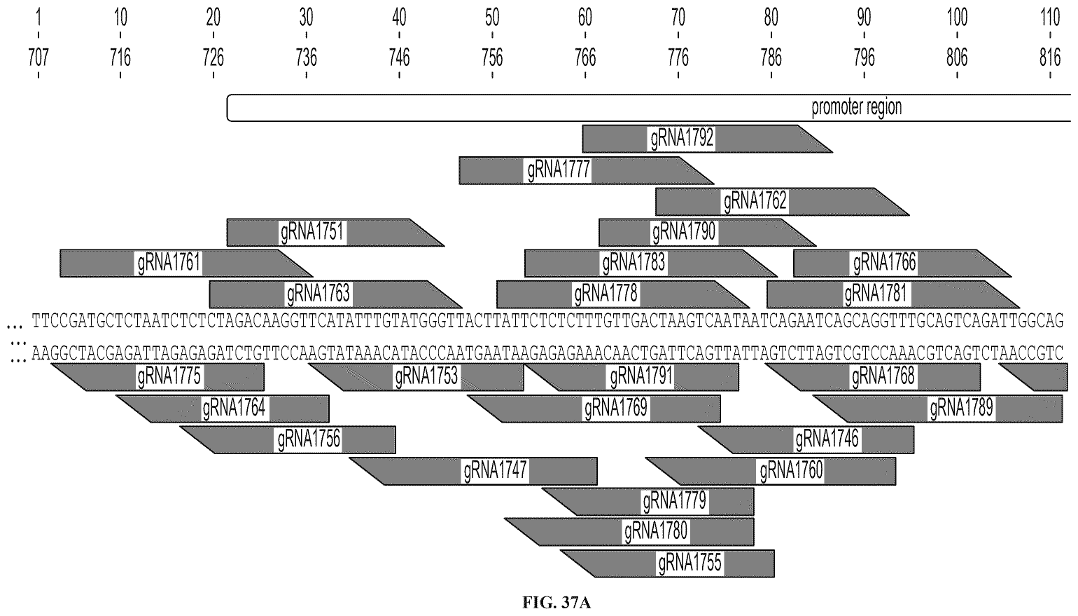

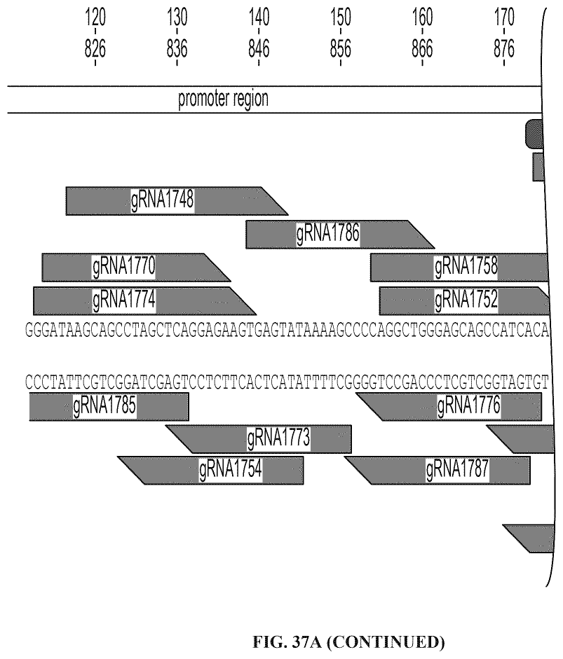

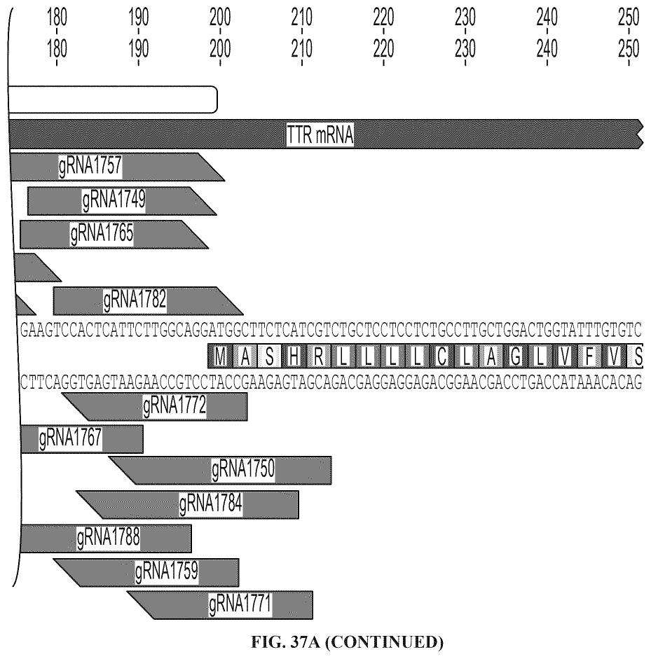

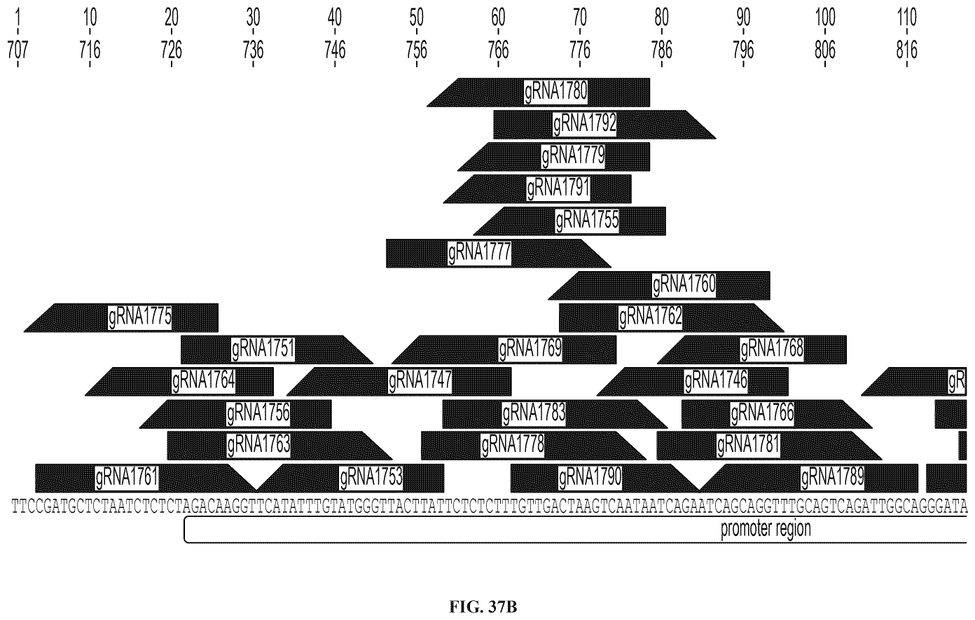

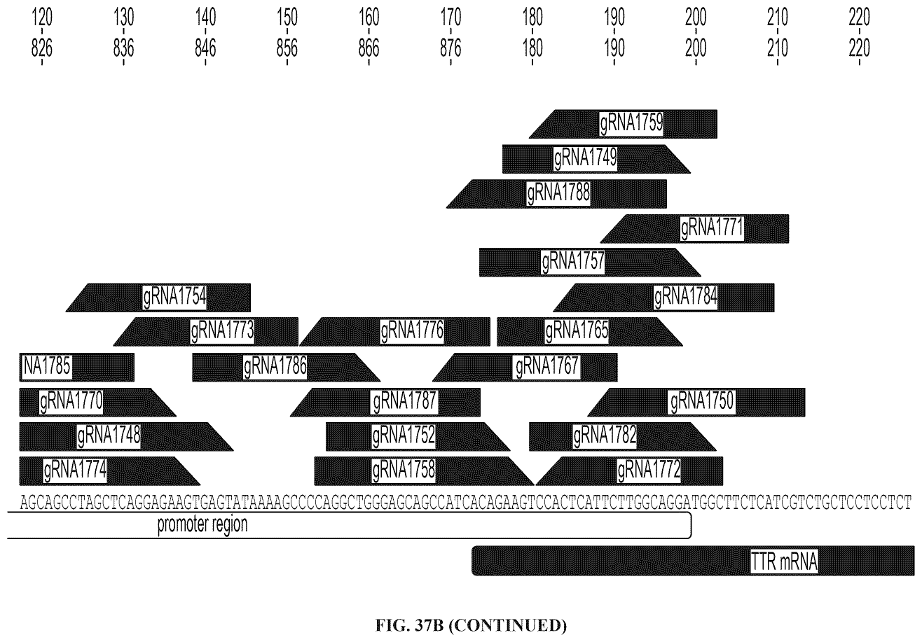

Sequence homology between the murine (see, Costa, R. H. & Grayson, D. R. Site-directed mutagenesis of hepatocyte nuclear factor (HNF) binding sites in the mouse transthyretin (TTR) promoter reveal synergistic interactions with its enhancer region. Nucleic Acids Res 19, 4139-4145 (1991), the disclosure of which is incorporated herein by reference in its entirety for all purposes; GGCAAGGTTCATATTTGTGTAGGTTACTTATTCTCCTTTTGTTGACTAAGTCAATAATCAGA ATCAGCAGG (SEQ ID NO: 1207)) and human TTR promoter regions was used to define the human promoter region to guide the design of guide RNA sequences for use in knocking out TTR in humans (FIGS. 37A and 37B).

gRNAs corresponding to four CRISPR-Cas enzymes with 3′ PAMs NGG, NGA, NNGRRT and NNNRRT were designed to tile the reported promoter region (FIGS. 37A and 37B). A base editing strategy was designed to generate mutations within the promoter region that would knock down TTR mRNA expression. 3′ NGG PAM gRNAs were designed to be paired with an S. pyogenes CRISPR-Cas9-containing base editor. 3′ NGA PAM gRNAs were designed to be paired with a mutated S. pyogenes CRISPR-Cas9-containing base editor. 3′ NNGRRT PAM gRNAs were designed to be paired with an S. aureus CRISPR-Cas9-containing base editor. 3′ NNNRRT PAM gRNAs were designed to be paired with a mutated S. aureus CRISPR-Cas9-containing base editor.

An in silico off-target analysis of these gRNAs was run and any gRNAs with a 0, 1, 2 or 3 nucleotide mismatch to a tumor suppressor gene were excluded from the screen due to potential off-target effects. The gRNA list was filtered further to remove any gRNAs with 0 or 1 mismatch to any location in the human genome and 0, 1 or 2 mismatches to any exon in the human genome. This filtered list contained 47 unique gRNAs that covering the target promoter region (FIGS. 37A and 37B). These 47 gRNAs could be paired with either an Adenine Base Editor (ABE) or Cytosine Base Editor (CBE) to make 94 unique guide-base editor type combinations.

Dna Editing Efficiency for gRNAs with Base Editors

A cellular screen for gRNA potency was undertaken. This screen used mRNA encoding for the base editor of interest and a chemically synthesized, chemically end-protected gRNA. The screening was performed in HepG2 human cells. Three replicates were transfected into cells on the same day. DNA was harvested for next generation sequencing three days post-transfection.

Positive controls for genome editing were the following: a gRNA-mRNA pair that was known to have good editing efficiencies and did not target DNA predicted to have any impact on TTR mRNA expression (sgRNA_088 paired with NGG-SpCas9-ABE8.8), three gRNA-base editor pairs targeting splice sites within the TTR gene (gRNAs sg_361, sg_362, gRNA1597 and gRNA1604), and one Cas9 nuclease combined with a gRNA known to be suitable for inducing TTR knockdown in human (Cas9 nuclease+gRNA991) (Gillmore, J. D. et al. CRISPR-Cas9 In Vivo Gene Editing for Transthyretin Amyloidosis. New Engl J Med 385, 493-502 (2021)).

Negative controls for genome editing were the following: no treatment, and a catalytically dead Cas9 nuclease plus gRNA991 (dead Cas9 nuclease+gRNA991).

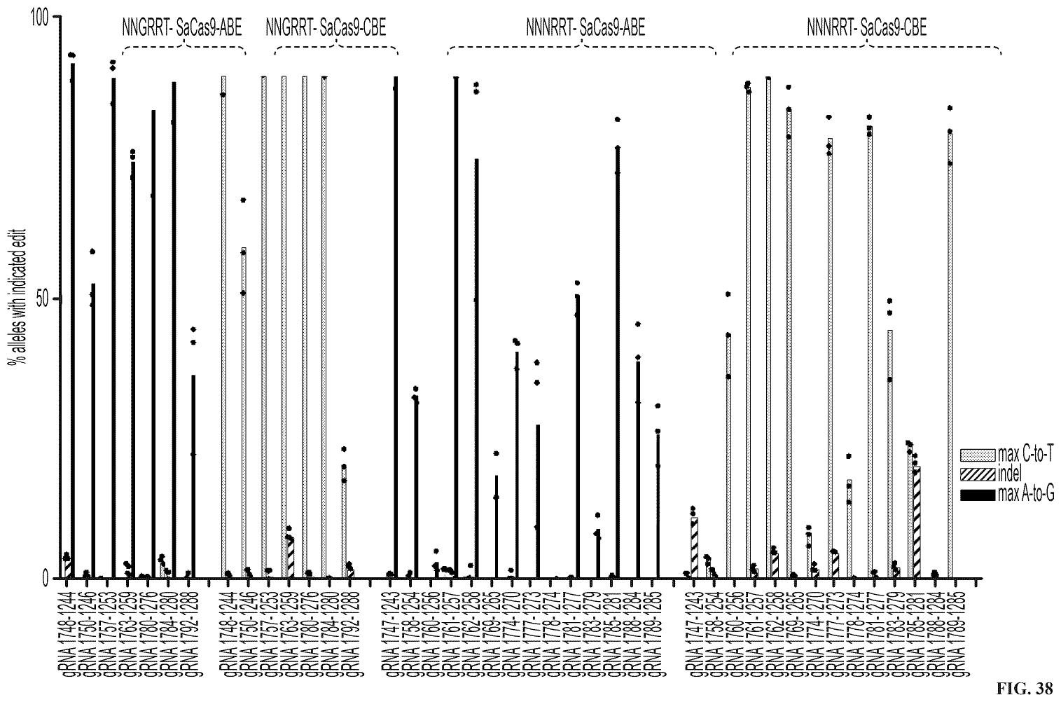

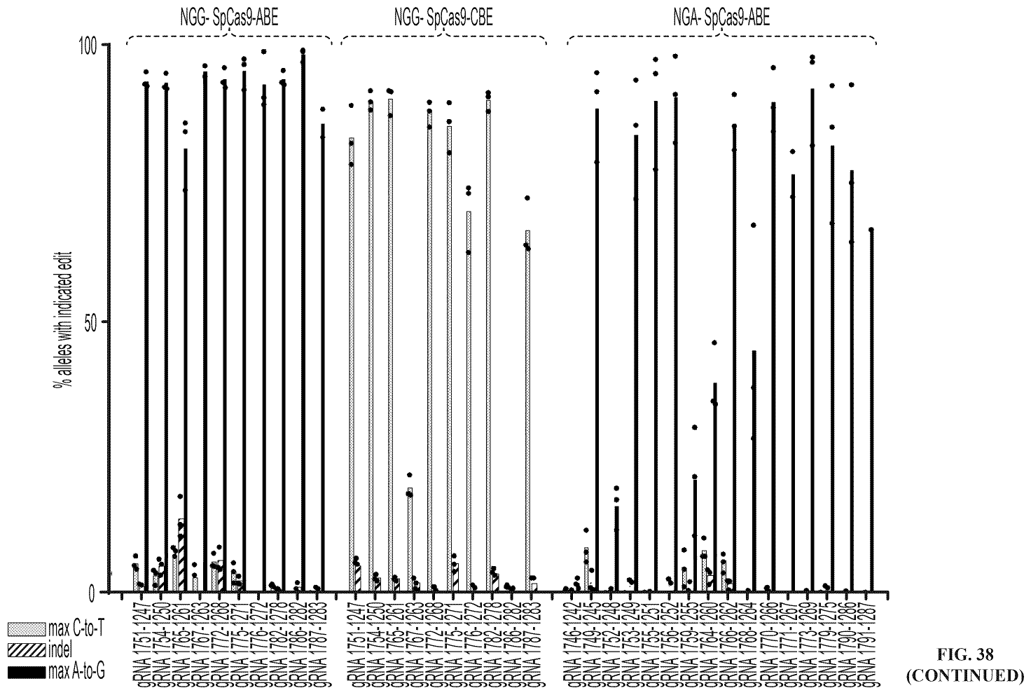

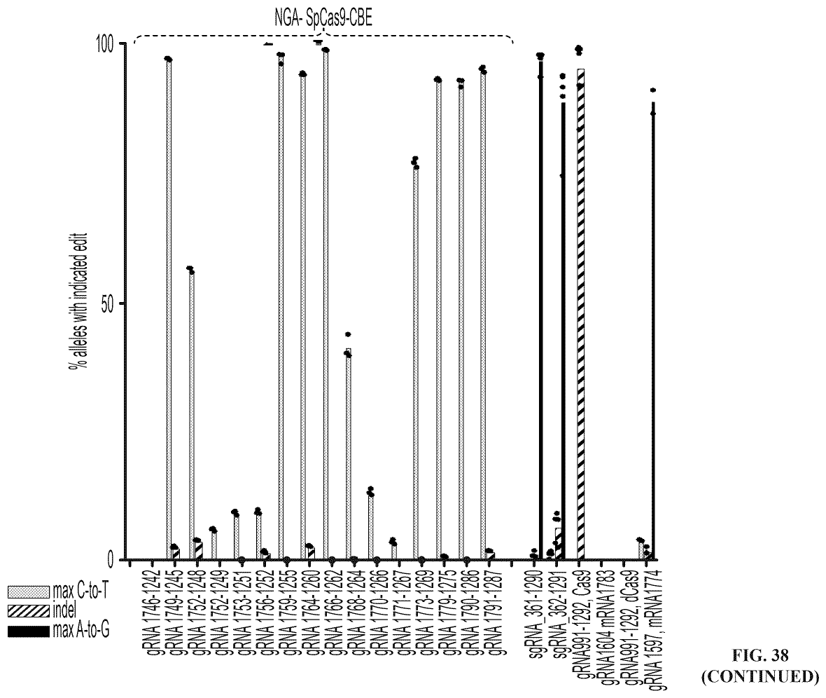

Each gRNA for the promoter screen was paired with either a CBE (here using the ppAPOBEC1 deaminase described in Yu, Y. et al. Cytosine base editors with minimized unguided DNA and RNA off-target events and high on-target activity. Nat Commun 11, 2052 (2020)) or an ABE (here using ABE8.20, described in Gaudelli, N. M. et al. Directed evolution of adenine base editors with increased activity and therapeutic application. Nat Biotechnol 38, 892-900 (2020)). Next-generation sequencing (NGS) data indicated that, when paired with CBEs, 22/46 promoter tiling gRNAs yielded mean editing frequencies >80% and 9/46 gRNAs yielded editing frequencies <10%. (FIG. 38). When paired with ABEs, 24/47 promoter tiling gRNAs yielded >80% mean editing frequency, and 4/47 gRNAs yielded mean editing frequencies <10%.

Ttr Knockdown Efficiency Resulting from Promoter Editing

TTR Knockdown efficiency was measured using RT-qPCR for all promoter screening gRNAs and the control gRNAs. One of the gRNAs that served as a positive control for DNA editing also served as a negative control for TTR knockdown: the gRNA-mRNA pair that typically yielded high editing efficiencies and did not target DNA known to have any impact on TTR mRNA expression (sgRNA_088 paired with NGG-SpCas9-ABE8.8). The other negative controls included no treatment controls, which were used in each plate run for RT-qPCR, and a catalytically dead Cas9 combined with gRNA_991.

Positive controls for TTR knockdown were the following: three previously identified gRNA-base editor pairs targeting splice sites within the TTR gene (gRNAs sg_361, sg_362, gRNA1597) and one Cas9 nuclease combined with a gRNA known to induce TTR knockdown in humans (Cas9 nuclease+gRNA991).

An internal control (ACTB) with an orthogonal fluorescent probe to the test probe (TTR) was used to enable RT-qPCR samples to be accurately compared between wells. Fold-change differences in TTR mRNA abundance between the no treatment controls and each test treatment well was measured using the mean of the ΔCt(TTR-ACTB)_{control} for the no treatment wells present in each plate. The approach used to find relative TTR expression level was 2∧(−1*(ΔCt (TTR-ACTB)ₛₐₘₚₗₑ-ΔCt(TTR-ACTB)_{control}) (Livak, K. J. & Schmittgen, T. D. Analysis of Relative Gene Expression Data Using Real-Time Quantitative PCR and the 2-ΔΔC T Method. Methods 25, 402-408 (2001)). Untreated cells had a different TTR: ACTB ratio from transfected cells, which led to an artificially reduced relative TTR expression (0.30-0.42) in cells transfected with the negative control catalytically dead Cas9 editor or gRNA that did not affect TTR expression. Nonetheless, this approach was suitable as a relative approach to compare TTR knockdown efficacy between different transfection conditions.

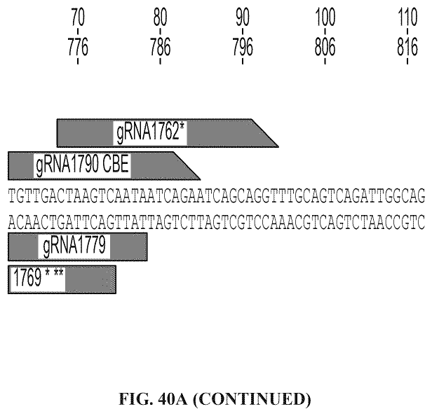

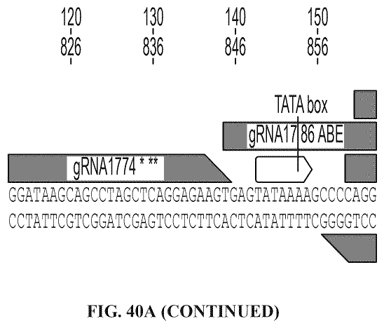

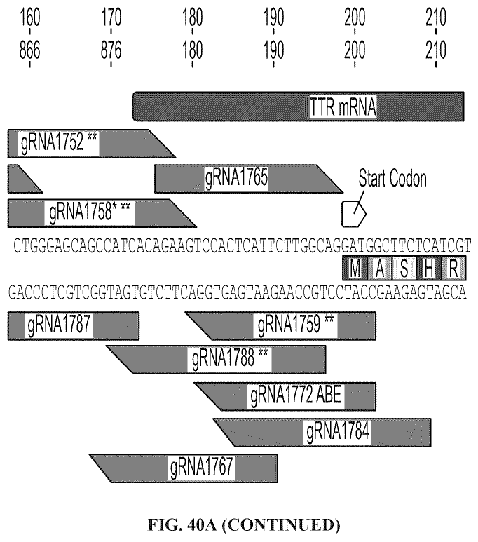

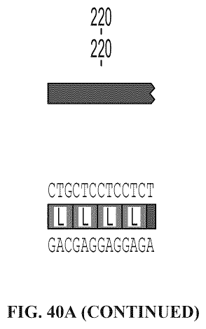

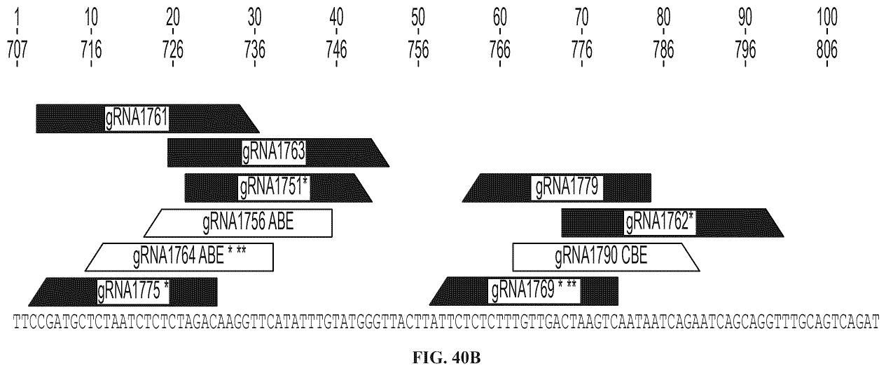

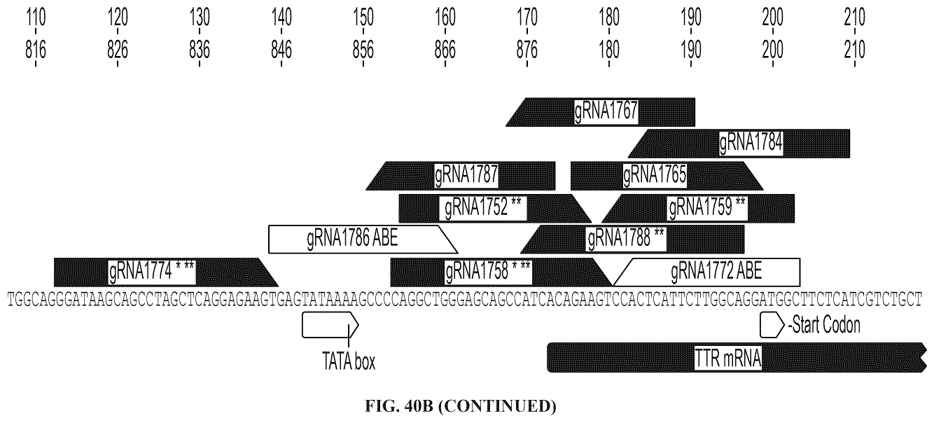

In total, 21/94 base editor-gRNA combinations (which are notated throughout this disclosure as “Base_Editor_Name_gRNA_name”) tested showed comparable or greater TTR knockdown than the positive control gRNA_991 (FIGS. 40A and 40B). Of five potent promoter tiling gRNAs, one, when combined with an ABE, edited the sequence proposed to be the TATA box for TTR (gRNA1786), and one, when combined with an ABE, disrupted the ATG start codon (gRNA1772). The other three bind elsewhere in the promoter region.

The following materials and methods were employed in this Example.

### Cell Transfection

HepG2 cells were plated into a 48-well poly-D-lysine (PDL)-coated plate (Corning, 354509) at a density of 25,000 cells/well in 200 μL of supplemented media 24-hours prior to transfection. On the day of transfection, 600 ng of mRNA encoding for the desired editor (produced at Beam) and 200 ng chemically end-protected gRNA (IDT) was aliquoted out and into 96-well plates. Lipofectamine MessengerMax (Thermo Fisher, LMRNA015) was diluted in Optimem (Thermo Fisher, 31985062), vortexed thoroughly and incubated at room temperature for at least 5 minutes before being added onto the pre-aliquoted mRNA and gRNA mix at a final concentration of 1.5 μL MessengerMax lipid per well. The lipid encapsulated mRNA and gRNA mix was incubated at room temperature for 10-20 mins before being added onto cell plates.

### Cell Culture

HepG2 cells (ATCC, HB8065) were cultured according to the manufacturer's protocols and split at least every four days. Cells were cultured in EMEM (Gibco, 670086), supplemented with 10% Fetal Bovine Serum (Thermo Fisher, A3160401).

### Next Generation DNA Sequencing (NGS)

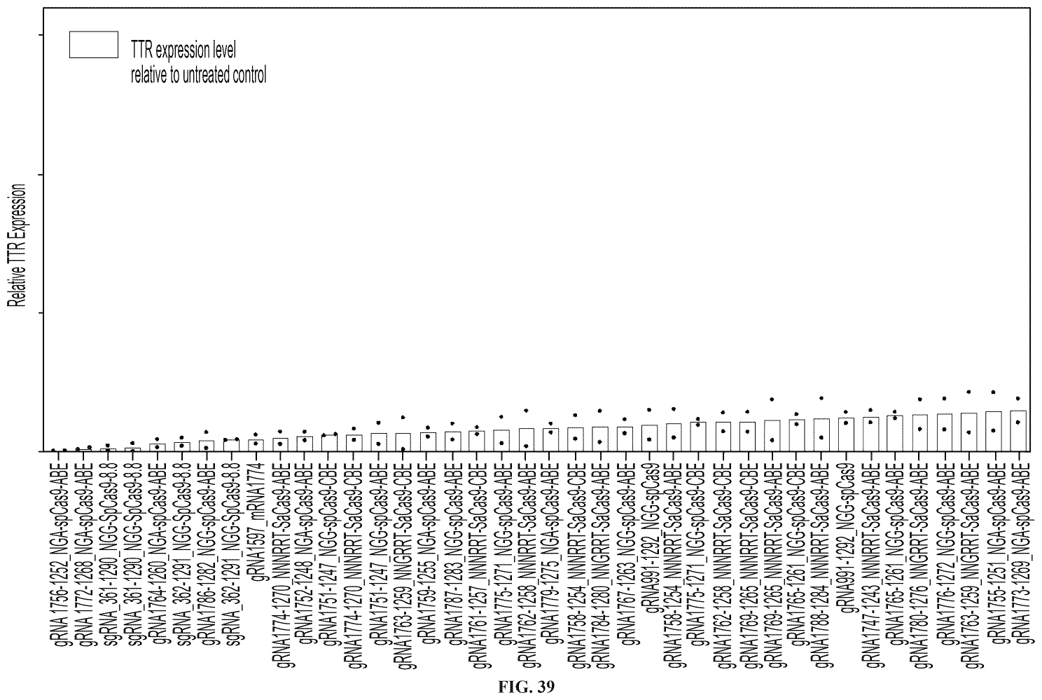

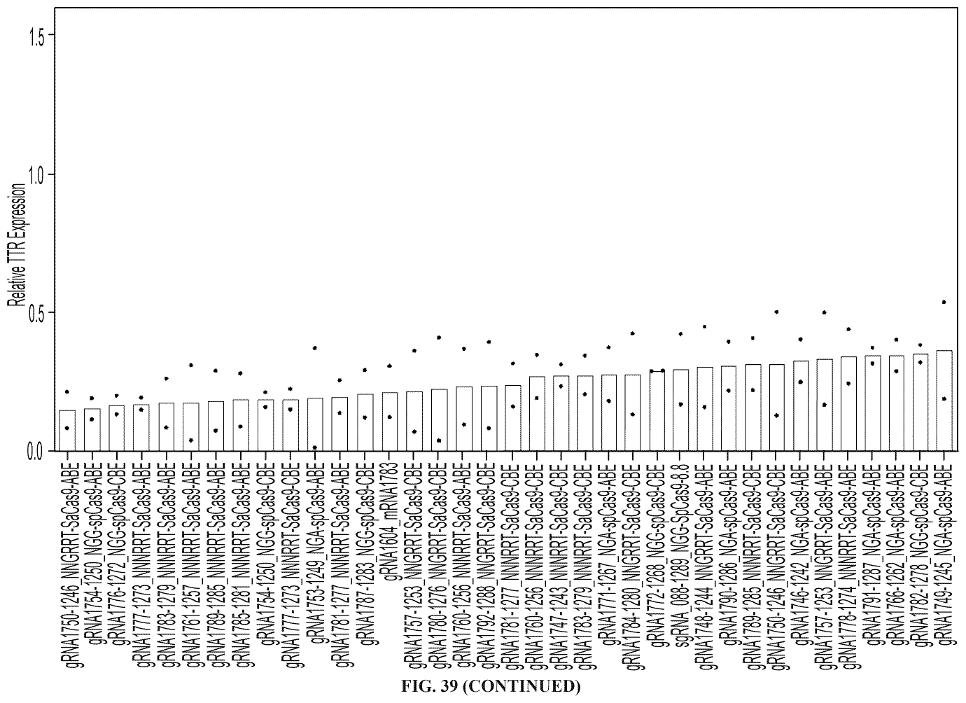

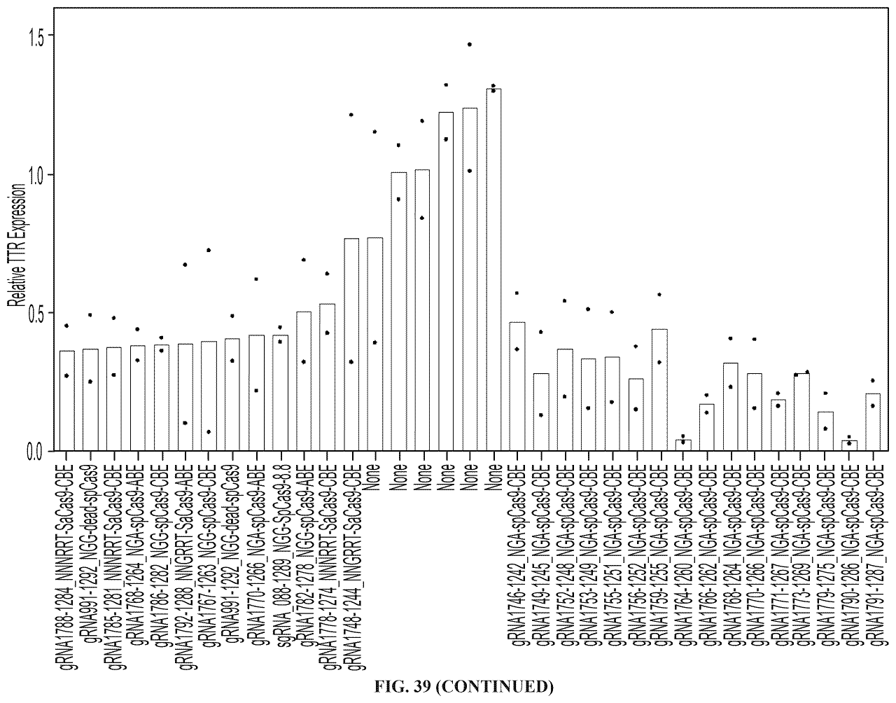

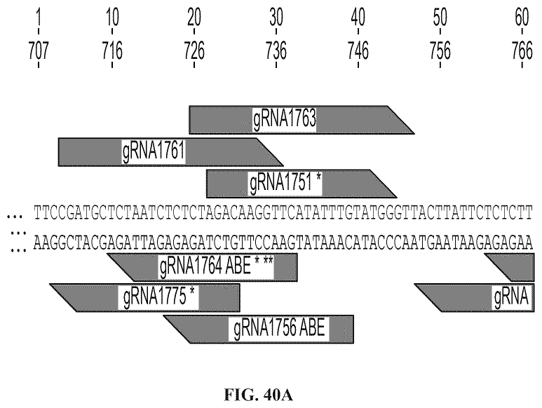

DNA was harvested from transfected cells 3 days post-transfection. Media was removed from cells and 100 μL of thawed Quick Extract lysis buffer (Lucigen, QEP70750) was added to each well. The buffer-cell mixture was incubated at 65° C. for 8 mins and then at 98° C. for 15 mins. PCR was performed to amplify the gRNA target region each sample. A second round of PCR was performed to add barcoding adapters onto the product from PCR1. The resulting product was purified and sequenced using a 300-kit on a Miseq (Illumina). DNA sequence alignment with a reference sequence and editing quantification was performed on the resulting sequences. Maximum editing (plotted in FIGS. 38 and 39) corresponded to the highest value for either an A-to-G edit or a C-to-T edit for any base within a gRNA protospacer and PAM region.

### RT-qPCR

Cells were frozen down 5 days post-transfection. Media was removed from each well and the resulting plates were sealed and stored at −80° C. RNA was harvested subsequently using the RNeasy PLUS kit (Qiagen) in 96 well plate format according to the manufacturer's instructions (74192). After RNA was isolated, Taqpath 1-step RT-qPCR Master Mix CG (Thermo Fisher, A15299) with two probes: ACTB with VIC(4448489) and TTR with FAM (4331182), all Thermo Fisher. The probes were used according to the manufacturer's instructions with 0.5 μL of RNA input in a 20 μL reaction to assess relative expression level of TTR. Quantstudio 7 (Thermo Fisher) was used to run the RT-qPCR assay. Three technical replicates were run per plate. Auto thresholds for Ct values were used for each individual value. Any replicates indicating no amplification or inconclusive amplification were excluded from the analysis, resulting in a few samples having only two technical replicates. To calculate relative expression of TTR, the ∧(—1*(ΔCt(TTR-ACTB)ₛₐₘₚₗₑ-ΔCt (TTR-ACTB)_{control}) approach (Livak, K. J. & Schmittgen, T. D. Analysis of Relative Gene Expression Data Using Real-Time Quantitative PCR and the 2-ΔΔC T Method. Methods 25, 402-408 (2001)) was used.

### Example 10: Transthyretin (TRR) Guide Screening and Functional Knockdown Assessment in Hek293T Cells

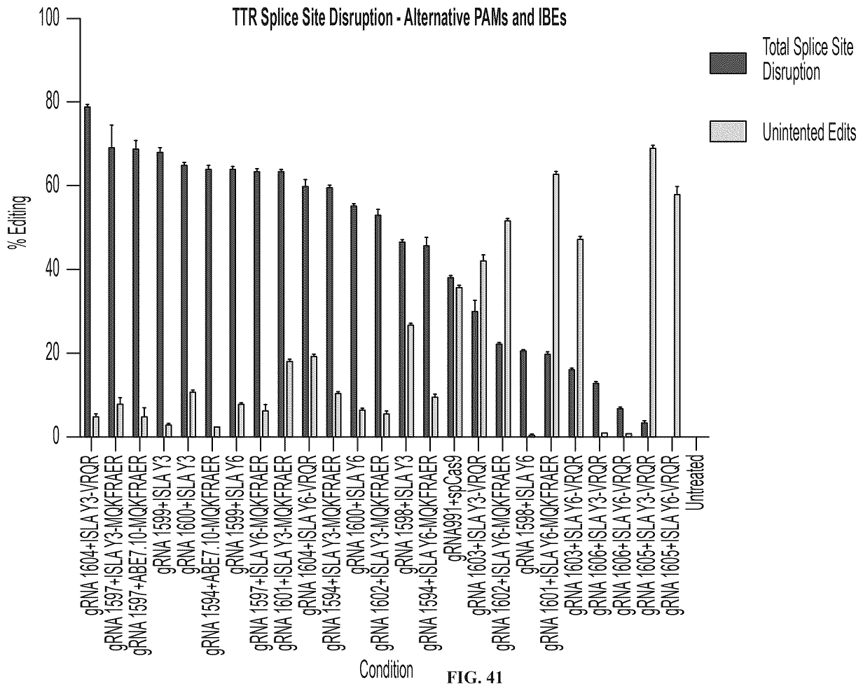

Fourteen guide RNAs were designed using a base-editing strategy for splice-site disruption using ABE7.10 alternative PAM editors or IBE variants, for a total of 26 new experimental combinations. Nine (9) tested combinations demonstrated good editing efficiencies in Hek293T cells (FIG. 41).

Editor mRNA and sgRNAs were transfected in triplicate into Hek293T cells. Spacer sequences for the sgRNAs are provided in Table 2B. All sgRNAs were ordered from IDT with 80-mer spCas9 scaffolds. In addition to the 26 experimental combinations, gRNA991 known to induce TTR knockdown in humans (Gillmore, J. D. et al. CRISPR-Cas9 In Vivo Gene Editing for Transthyretin Amyloidosis. New Engl J Med 385, 493-502 (2021)) combined with spCas9, and no treatment were used as positive and negative controls, respectively. Genomic DNA was harvested 72 hours after transfection and sequenced using Next Generation Sequencing. Total editing resulting in splice site disruption was detected in a range from ˜79%-0.4% depending on the condition, with some combinations yielding total editing resulting in splice disruption in a range between 79% to 63.5%. Most editor variants exhibited detectable editing at target loci. The following combinations displayed relatively high levels of editing: ISLAY3-VRQR gRNA1604; ISLAY3-MQKFRAER_gRNA1597; ABE7.10-MQKFRAER_gRNA1597; ISLAY3_gRNA1599; ISLAY3_gRNA1600; ABE7.10-MQKFRAER_gRNA1594; ISLAY6_gRNA1599; ISLAY6-MQKFRAER_gRNA1597; ISLAY3-MQKFRAER gRNA1601. For a description of the internal base editors (ISLAY) see Tables 4A and 4B. The internal base editors (i.e., ISLAY3 and ISLAY6, each contained a TadA*7.10 deaminase domain. The internal base editors are described in PCT/US20/16285, the disclosure of which is incorporated herein by reference in its entirety for all purposes. In particular, the combination of gRNA1604 and ISLAY3-VRQR exhibited editing efficiencies at ˜79%. The combination of gRNA1597 with both ISLAY3-MQKFRAER and ABE7.10 exhibited good editing efficiencies as well.

The following materials and methods were employed in this Example.

### Hek293T Cell Culture and Maintenance

A frozen vial of Hek293T cells at passage count 3 was thawed and mixed with 15 mL of pre-warmed DMEM high glucose pyruvate medium (Thermofisher, 11995065) with 10% Fetal Bovine Serum (Thermofisher, A³¹⁶⁰⁴⁰¹) and Pen/Strep (Thermofisher, 10378016), and plated on a T75 tissue-culture treated flask (Corning, 430641U) at 37° C. in a 5% CO₂ incubator (Thermofisher 51033547). The media was aspirated and replaced the next morning, and every other day thereafter. Upon reaching 70-80% confluency after 3 days, the cells were split at 1:20 via aspiration of media followed by incubation with 2 mL TrypLE (Thermofisher 12605036) for 3 minutes, gentle agitation and pipette mixing, and transfer of 100 μL into 15 mL pre-warmed media again. This process was repeated after another 5 days, during which time cell counts were obtained by averaging two results obtained from a NucleoCounter NC-200 after diluting the 2 mL of TrypLE cell suspension obtained from the flask in 10 mL of media. The cells were then seeded into Poly-D-Lysine 48-well plates (Corning, 354509) at 25kcells/well in 200 μL of media.

### Cell Transfection

Hek293T cells were transfected the day after seeding. The media was changed prior to transfection. Each well received 200 ng gRNA (Synthego custom order) (sequences for the guide RNA's are provided in Tables 1 and 2B; gRNA991 contained the spacer sequence AAAGGCUGCUGAUGACACCU (SEQ ID NO: 1205) and 600 ng mRNA with 1.5 μL Lipofectamine MessengerMax (Thermofisher, LMRNA150). Guide RNAs were reconstituted from lyophilized form in water at 1 mg/mL, and mRNA was received at 2 mg/mL. gRNA/mRNA and reagent were separately added to 26 μL OptiMEM (Thermofisher, 31985062) per well as half mixes and incubated for 10 minutes, after which the RNA and reagent half mixes were combined and incubated for another 5 min. 54 μL of the combined mastermix was added dropwise to each target culture well. The plates were then briefly and gently nutated and placed at 37° C. and 5% CO₂ in the incubator. Media was changed the following day.

### Next Generation DNA Sequencing (NGS)

72 hours after transfection, media was aspirated and genomic DNA was isolated with lysis buffer solution of 10 mM Tris-HCl pH8.0, 0.05% SDS, 50 ug/mL proteinase K (Thermofisher, EO0491). 200 μL of lysis buffer was added per well, and the plates were incubated at 37° C. for 45 minutes, after which the samples were vigorously mixed and 100 μL of the volume was transferred to a 96-well PCR plate. The plate was incubated at 95° C. for 15 minutes and 1 μL was transferred into a PCR mixture. PCR was performed using Q5 Hotstart 2x Mastermix (M0494L) and target site-specific amplicon primers. 25 μL of mastermix, 5 uM each of forward and reverse primer, and to 50 μL of water were used per well. A second round of barcoding PCR was performed with half the volume. PCR products were pooled by amplicon sequence and 166 μL was added to 33 μL Purple 6x Dye (B7024S) and gel extracted in 1% agarose, then purified twice using Zymo Gel Extraction (D4007) and PCR Cleanup (D4013) kits, eluting in 150 μL 10 mM Tris pH7.5. The library concentration was quantified via NanoDrop (Thermofisher, ND-ONE-W), and standardized to 4 nM. Sequencing was performed using a MiSeq Reagent Kit v2 (500 cycles) (Illumina, MS-102-2003), with read alignment to reference sequences and editing efficiency was computationally analyzed.

### Example 11: In Vivo Non-Human Primate (NHP) Base Editing of TTR Gene

In this example, NHP surrogate sgRNA (GA519, SEQ ID NO. 1044), corresponding to the human sgRNA described above, was prepared, and formulated with ABE8.8 mRNA, encapsulated in lipid nanoparticles (LNP 11 and LNP 12), and intravenously dosed to NHPs. Some objectives of this study were to determine the base editing of LNPs encapsulating mRNA and a single guide RNA, when given intravenously once on Day 1 to cynomolgus monkeys. Both LNP 11 and LNP 12 encapsulate sgRNA (GA519, SEQ ID NO. 1044) and ABE8.8 mRNA. LNP 11 and LNP 12 differed most notably in the ionizable lipids (BLP8-4 vs. LP01) and in the presence of a GalNAc ligand component in LNP 12 formulation. The components of LNP 11 and LNP 12 are indicated in Tables 26 and 27 below, respectively.

TABLE 26
LNP 11 Components

| LNP | | | |
|---|---|---|---|
| 11 Component | Name | Structure | Mol % |
| Amino lipid (BLP8-4) | ((3-hydroxypropyl)azanediyl)bis(heptane- 7,1-diyl)bis(4,4-bis(((Z)-oct-5- en-1-yl)oxy)butanoate)^{##} | 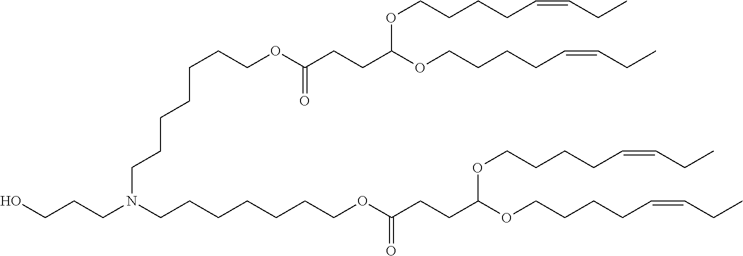 [SMILES: CC/C=C\CCCCOC(CCC(=O)OCCCCCCCN(CCCO)CCCCCCCOC(=O)CCC(OCCCC/C=C\CC)OCCCC/C=C\CC)OCCCC/C=C\CC] | 47.5 |
| Neutral helper lipid | 1,2-distearoyl-sn-glycero-3-phospho- choline (DSPC) | 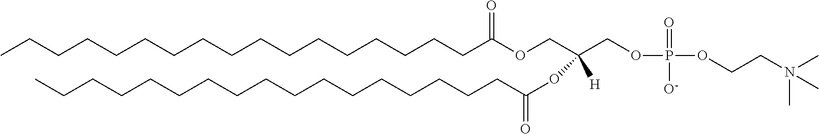 | 10 |
| PEG-lipid | 1,2-dimyristoyl-rac-glycero-3-methoxy- polyethylene glycol-2000 (PEG₂₀₀₀-DMG) | 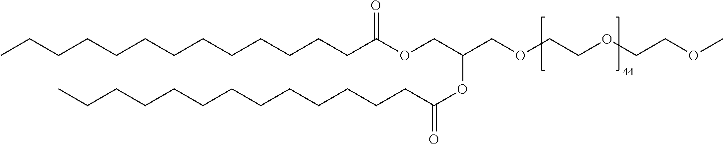 | 2.5 |
| Sterol lipid | Cholesterol | 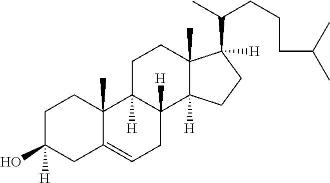 | 40 |

^{##}described in International Published Patent Application WO 2022 140252

TABLE 27
LNP 12 Components

| LNP 12 | | | |
|---|---|---|---|
| Component | Name | Structure | Mol % |
| Amino lipid | 3-((4,4-bis(octyloxy)butanoyl)oxy)- 2-((((3- (diethylamino)propoxy)carbonyl) oxy)methyl)propyl(9Z,12Z)- octadeca-9,12-dienoate* | 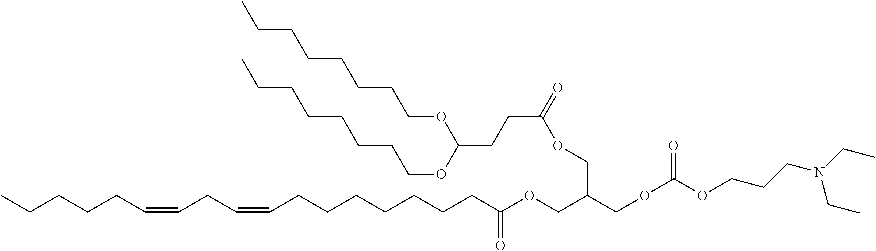 [SMILES: CCCCC/C=C\C/C=C\CCCCCCCC(=O)OCC(COC(=O)CCC(OCCCCCCCC)OCCCCCCCC)COC(=O)OCCCN(CC)CC] | 50 |
| Neutral helper lipid | 1,2-distearoyl-sn-glycero-3- phosphocholine (DSPC) | 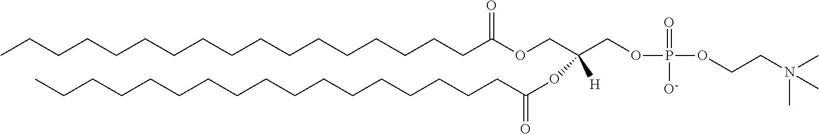 | 9 |
| PEG-lipid | 1,2-dimyristoyl-rac-glycero-3- methoxypolyethylene glycol-2000 (PEG₂₀₀₀-DMG) | 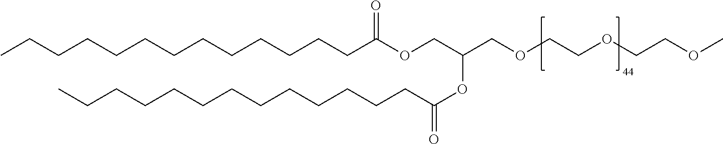 | 3 |
| Sterol lipid | Cholesterol | 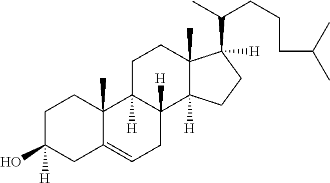 | 37.95 |
| GalNAc- lipid | N²-(PEG-DSG)-N⁶-((C5- GalNAc)amido)-Lys-[bis((C5- GalNAc)propylamido)]amide or DSG-PEG-Lys-tris(GalNAc)** | 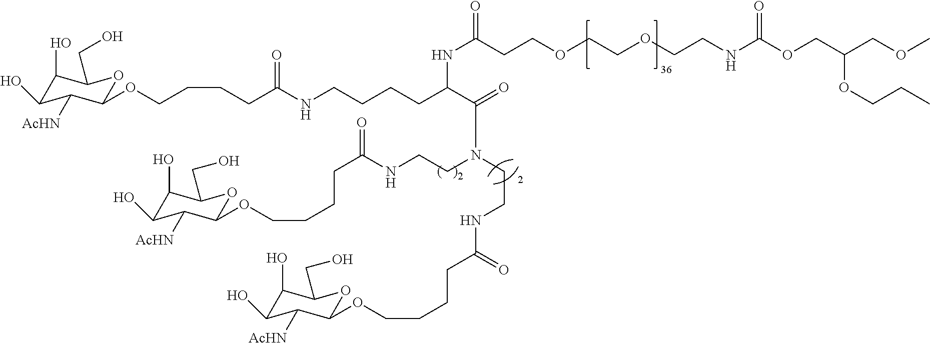 | 0.05 |

*described in International Published Patent Application WO 2015 095340 A1
**described in International Published Patent Application WO 2021/178725 A1

It should be understood that the mol % of components in Tables 26 and 27 may be adjusted and that the mol % included in Tables 26 and 27 are targeted excipient percentages of the LNP, which is intended to represent the aggregate mol % of all the LNPs formulated in a given batch and that specific LNPs within a batch may have varying mol %. Thus, it is contemplated herein that the mol % of one or more, or all of the LNP components set forth in Tables 26 and 27 may be adjusted, for example, by +/−1-5%, +/−5-10%, or +/−10%-20%. It is further contemplated herein that the mol % of one or more, or all of the LNP components set forth in Tables 26 and 27 with respect to a specific LNP formulated in a given batch of LNPs formulated in accordance with desired target excipient percentages, may vary from the targeted mol %, for example, by +/−1-5%, +/−5-10%, or +/−10%-20%, or even greater than +/−20%. Further, it should be understood that additional LNP components, including non-lipid components, may be added to the LNP components set-forth in Tables 26 and 27. LNP 11 and LNP 12 were formulated with an sgRNA: mRNA weight ratio of 1:1. In other words, the LNPs were formulated with an equal amount by weight of guide RNA as mRNA. The resulting LNPs encapsulating the sgRNA and mRNA were filtered using 0.22 micron filters.

### NHP Study Design

In this aspect of the study, male cynomolgus monkeys of Cambodian origin were used as study animals. A premedication regimen comprising dexamethasone and antihistamines (diphenylhydramine and famotidine) was administered to all animals on day-1 and day 1 (predose), at 30 to 60 minutes prior to test article dose administration. Three monkeys were dosed with LNP 11, and three monkey were dosed with LNP 12 on day 1 of the study via a single IV infusion at a dose level of 1 mg of combined sgRNA and mRNA per kg of animal body weight and at a dose volume of 6 mL/kg (n=3/group).

Blood samples were collected from all animals predose for baseline measurements and post-dose at various time points on days 1 through 8 to assess biomarkers, cytokines, pharmacokinetics, and serum safety parameters. Necropsies were performed on all animals at day 8. Liver biopsy samples were collected to assess TTR gene editing.

### Analysis of Editing Efficiency

The amount of gene editing in the liver was evaluated by next-generation sequencing of targeted PCR amplicons at the TTR target site. Editing was reported as the percent measure of the editing rate of the A within the ATG start codon. Table 28 below shows the TTR editing efficiency in liver of LNP 11 as compared to LNP 12. The average hepatic TTR editing efficiency (specific allele) is higher in NHP treated with LNP 11 (34.5%) compared to LNP 12 (28.9%).

TABLE 28
Hepatic TTR Editing Efficiency of LNP 11 and LNP 12

| | Dose | | | | | |
|---|---|---|---|---|---|---|
| Product | (mg/kg) | Animal 1 | Animal 2 | Animal 3 | Mean | SD |
| LNP 11 | 1 | 41.6 | 39.2 | 22.6 | 34.5 | 10.4 |
| LNP 12 | 1 | 31.7 | 34.9 | 20.0 | 28.9 | 7.8 |

### Quantification of TTR Protein Expression in Plasma

TTR protein collected from plasma was quantitated using LC-MS, in which four unique TTR peptide fragments were quantitated from each sample at various time points. Normalized percent plasma cTTR (4-peptide average) at terminal (day 8) vs. pre-dose levels is provided in Table 29 below. LNP 11 showed greater plasma TTR reductions (−38% change from baseline on day 8) when compared to LNP 12 (−12% change from baseline on day 8).

TABLE 29
cTTR Plasma Levels at Terminal vs. Pre-dose

| | Dose | | | | | |
|---|---|---|---|---|---|---|
| Product | (mg/kg) | Animal 1 | Animal 2 | Animal 3 | Mean | SD |
| LNP 11 | 1 | 48% | 53% | 84% | 62% | 19% |
| LNP 12 | 1 | 67% | 90% | 108% | 88% | 16% |

Thus, infusion of LNP 11 and LNP 12 in NHPs resulted in editing of the TTR gene in the liver, with LNP 11 demonstrating greater editing than LNP 12. The greater editing of LNP 11 in NHPs corresponded to a commensurate increase in the reduction in plasma TTR concentrations in NHPs.

### Example 12: In Vivo Non-human Primate (NHP) Base Editing of TTR Gene

In this example, NHP surrogate sgRNA (GA519, SEQ ID NO. 1044), corresponding to the human sgRNA described above, was prepared, and formulated with ABE8.8 mRNA, encapsulated in a lipid nanoparticle (LNP 13), and intravenously dosed to NHPs. Objectives of this study included determining the base editing of LNP 13 encapsulating mRNA and a single guide RNA, when given intravenously once on Day 1 to cynomolgus monkeys. The components of LNP 13 are indicated in Table 30 below.

TABLE 30
LNP 13 Components

| LNP | | | |
|---|---|---|---|
| 13 Component | Name | Structure | Mol % |
| Amino lipid (BLP4-71) | 1-(3-((4,4-bis(((Z)-oct-5-en-1- yl)oxy)butanoyl)oxy)-2-(((((1- ethylpiperidin-3- yl)methoxy)carbonyl)oxy)methyl)pro- pyl) 7-(3-pentyloctyl) heptanedioate^{$$} | 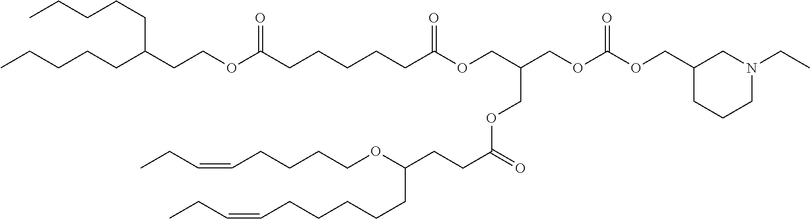 [SMILES: CC/C=C\CCCCCC(CCC(=O)OCC(COC(=O)CCCCCC(=O)OCCC(CCCCC)CCCCC)COC(=O)OCC1CCCN(CC)C1)OCCCC/C=C\CC] | 47.5 |
| Neutral helper lipid | 1,2-distearoyl-sn-glycero-3-phosphocholine (DSPC) | 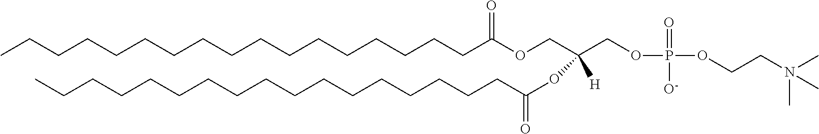 | 10 |
| PEG-lipid | 1,2-dimyristoyl-rac-glycero-3- methoxypolyethylene glycol-2000 (PEG₂₀₀₀-DMG) | 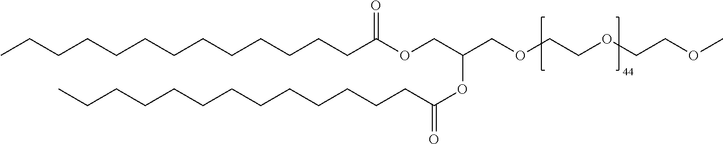 | 2.5 |
| Sterol lipid | Cholesterol | 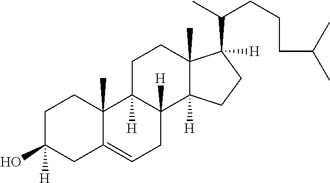 | 40 |

^{$$}described in International Published Patent Application WO 2022 159472

It should be understood that the mol % of components in Table 30 may be adjusted and that the mol % included in Table 30 are targeted excipient percentages of the LNP, which is intended to represent the aggregate mol % of all the LNPs formulated in a given batch and that specific LNPs within a batch may have varying mol %. Thus, it is contemplated herein that the mol % of one or more, or all of the LNP components set forth in Table 30 may be adjusted, for example, by +/−1-5%, +/−5-10%, or +/−10%-20%. It is further contemplated herein that the mol % of one or more, or all of the LNP components set forth in Table 30 with respect to a specific LNP formulated in a given batch of LNPs formulated in accordance with desired target excipient percentages, may vary from the targeted mol %, for example, by +/−1-5%, +/−5-10%, or +/−10%-20%, or even greater than +/−20%. Further, it should be understood that additional LNP components, including non-lipid components, may be added to the LNP components set-forth in Table 30.

LNP 13 was formulated with an sgRNA: mRNA weight ratio of 1:1. In other words, the LNP was formulated with an equal amount by weight of guide RNA as mRNA. The resulting LNP encapsulating the sgRNA and mRNA was filtered using 0.22 micron filters.

### NHP Study Design

In this aspect of the study, male cynomolgus monkeys of Cambodian origin were used as study animals. A premedication regimen comprising dexamethasone and antihistamines (diphenylhydramine and famotidine) was administered to all animals on day-1 and day 1 (predose), at 30 to 60 minutes prior to test article dose administration. Three monkeys were dosed with LNP 13 on day 1 of the study via a single IV infusion at a dose level of 2 mg of combined sgRNA and mRNA per kg of animal body weight and at a dose volume of 6 mL/kg (n=3/group). Blood samples were collected from all animals predose for baseline measurements and post-dose at various time points on days 1 through 15 to assess biomarkers, cytokines, pharmacokinetics, and serum safety parameters. Necropsies were performed on all animals at day 15. Liver biopsy samples were collected to assess TTR gene editing.

### Analysis of Editing Efficiency

The amount of gene editing in the liver was evaluated by next-generation sequencing of targeted PCR amplicons at the TTR target site. Editing was reported as the percent measure of the editing rate within the start codon. Table 31 below shows the TTR editing efficiency in liver of LNP 13. The average hepatic TTR editing efficiency for LNP 13 was 20.8%.

TABLE 31
Hepatic TTR Editing Efficiency of LNP 13

| | Dose | | | | | |
|---|---|---|---|---|---|---|
| Product | (mg/kg) | Animal 1 | Animal 2 | Animal 3 | Mean | SD |
| LNP 13 | 2 | 17.2 | 31.3 | 13.9 | 20.8 | 9.2 |

### Quantification of TTR Protein Expression in Plasma

TTR protein collected from plasma was quantitated using LC-MS, in which four unique TTR peptide fragments were quantitated from each sample at various time points. Normalized percent plasma cTTR (4-peptide average) at terminal (day 15) vs. pre-dose levels is provided in Table 32 below. LNP 13 showed plasma TTR reduction of −23% change from baseline on day 15.

TABLE 32
cTTR Plasma Levels at Terminal vs. Pre-dose

| | Dose | | | | | |
|---|---|---|---|---|---|---|
| Product | (mg/kg) | Animal 1 | Animal 2 | Animal 3 | Mean | SD |
| LNP 13 | 2 | 63% | 47% | 120% | 77% | 39% |

Thus, infusion of LNP 13 in NHPs resulted in editing of the TTR gene in the liver and reduction in serum TTR concentrations.

The following materials and methods were employed in Examples 5-10.

### General HEK293T Mammalian Culture Conditions

Cells were cultured at 37° C. with 5% CO₂. HEK293T cells [CLBTx013, American Type Cell Culture Collection (ATCC)] were cultured in Dulbecco's modified Eagles medium plus Glutamax (10566-016, Thermo Fisher Scientific) with 10% (v/v) fetal bovine serum (A31606-02, Thermo Fisher Scientific). Cells were tested negative for mycoplasma after receipt from supplier.

### Lipotransfection

HEK293T cells were seeded onto 48-well well Poly-D-Lysine treated BioCoat plates (Corning) at a density of 35,000 cells/well and transfected 18-24 hours after plating. Cells were counted using a NucleoCounter NC-200 (Chemometec). A solution was prepared containing Opti-MEM reduced serum media (ThermoFisher Scientific), the base editor, nuclease, or control mRNA, and sgRNA. The solution was combined with Lipofectamine MessengerMAX (ThermoFisher) in Opti-MEM reduced serum media and left to rest at room temperature for 15 min. The resulting mixture was then transferred to the pre-seeded Hek293T cells and left to incubate for about 120 h.

### Dna Extraction and Analysis of Editing

Cells were harvested and DNA was extracted. For DNA analysis, cells were washed once in 1×PBS, and then lysed in 100 μl QuickExtract™ Buffer (Lucigen) according to the manufacturer's instructions.

Genomic DNA was sequences using Illumina Miseq sequencers following PCR to amplify edited regions.

mRNA Production

All base editor and bhCas12b mRNA was generated using the following synthesis protocol. Base editors or bhCas12b were cloned into a plasmid encoding a dT7 promoter followed by a 5′UTR, Kozak sequence, ORF, and 3′UTR. The dT7 promoter carries an inactivating point mutation within the T7 promoter that prevents transcription from circular plasmid. This plasmid templated a PCR reaction (Q5 Hot Start 2X Master Mix), in which the forward primer corrected the SNP within the T7 promoter and the reverse primer appended a poly A tail to the 3′ UTR. The resulting PCR product was purified on a Zymo Research 25 ug DCC column and used as mRNA template in the subsequent in vitro transcription. The NEB HiScribe High-Yield Kit was used according to the instruction manual, but with full substitution of N1-methyl-pseudouridine for uridine and co-transcriptional capping with CleanCap AG (Trilink). Reaction cleanup was performed by lithium chloride precipitation. Primers used for amplification can be found in Table 33.

TABLE 33
Primers used for ABE8 T7 in vitro transcription reactions

| Name | Sequence |
|---|---|
| fwd_IVT | TCGAGCTCGGTACCTAATACGACTCAC (SEQ ID NO: 1208) |
| rev_IVT | TTTTTTTTTTTTTTTTTTTTTTTTTTTTTTTTTTTTTTTTTTTTTTTTTTTTT |
| | TTTTTTTTTTTTTTTTTTTTTTTTTTTTTTTTTTTTTTTTTTTTTTTTTTTTT |
| | TTTTTTTTTTTTTTCTTCCTACTCAGGCTTTATTCAAAGACCA (SEQ ID NO: |
| | 1209) |

TABLE 34
gRNA spacer sequence with PS linkage at 5′ end
gRNA spacer sequence (5′-3′)

gscscsAUCCUGCCAAGAAUGAG (SEQ ID
NO: 472)
gscscsAUCCUGCCAAGAACGAG (SEQ ID
NO: 1210)
gscsasACUUACCCAGAGGCAAA (SEQ ID
NO: 1211)
usasusAGGAAAACCAGUGAGUC (SEQ ID
NO: 1212)
usascsUCACCUCUGCAUGCUCA (SEQ ID
NO: 1213)
gscscsAUCCUGCCAAGAACGAG (SEQ ID
NO: 1210)

wherein: A is adenosine; C is cytidine; G is guanosine; U is uridine; a is 2′-O-methyladenosine; c is 2′-O-methylcytidine; g is 2′-O-methylguanosine; u is 2′-O-methyluridine and s is phosphorothioate (PS) backbone linkage.

TABLE 35
gRNA spacer sequence without PS linkage
gRNA spacer sequence (5′-3′)

GCCAUCCUGCCAAGAAUGAG (SEQ ID NO:
472)
GCCAUCCUGCCAAGAACGAG (SEQ ID NO:
473)
GCAACUUACCCAGAGGCAAA (SEQ ID NO:
474)
UAUAGGAAAACCAGUGAGUC (SEQ ID NO:
475)
UACUCACCUCUGCAUGCUCA (SEQ ID NO:
476)

wherein A is a modified or unmodified adenosine; C is a modified or unmodified cytidine; G is modified or unmodified guanosine; and U is a modified or unmodified uridine

TABLE 36
Guide RNA sequence
Guide RNA sequence (5′-3′)

gscscsAUCCUGCCAAGAAUGAGGUUUUAGAGCUAGAAAUAGCAAGUUAAAAUAAGGCUAG
UCCGUUAUCAACUUGAAAAAGUGGCACCGAGUCGGUGCUsususu (SEQ ID NO:
479)
AUCCUGCCAAGAACGAGGUUUUAGAGCUAGAAAUAGCAAGUUAAAAUAAGGCUAGUCCGUU
AUCAACUUGAAAAAGUGGCACCGAGUCGGUGCUsususu (SEQ ID NO: 1214)
gscsasACUUACCCAGAGGCAAAGUUUUAGAGCUAGAAAUAGCAAGUUAAAAUAAGGCUAG
UCCGUUAUCAACUUGAAAAAGUGGCACCGAGUCGGUGCUsususu (SEQ ID NO:
499)
usasusAGGAAAACCAGUGAGUCGUUUUAGAGCUAGAAAUAGCAAGUUAAAAUAAGGCUAG
UCCGUUAUCAACUUGAAAAAGUGGCACCGAGUCGGUGCUsususu (SEQ ID NO:
497)
usascsUCACCUCUGCAUGCUCAGUUUUAGAGCUAGAAAUAGCAAGUUAAAAUAAGGCUAG
UCCGUUAUCAACUUGAAAAAGUGGCACCGAGUCGGUGCUsususu (SEQ ID NO:
480)
gscscsAUCCUGCCAAGAACGAGgUUUUAGagcuaGaaauagcaaGUUaAaAuAaggcuaG
UccGUUAucAAcuuGaaaaagugGcaccgagucggugcuususus (SEQ ID NO:
1044)
usasusAGGAAAACCAGUGAGUCgUUUUAGagcuaGaaauagcaaGUUaAaAuAaggcuaG
UccGUUAucAAcuuGaaaaagugGcaccgagucggugcuususus (SEQ ID NO:
497)
wherein A is adenosine; C is cytidine; G is guanosine; U is uridine; a is 2′-O-methyladenosine; c is 2′-O-methylcytidine; g is 2′-O-methylguanosine; u is 2′-O-methyluridine and s is phosphorothioate (PS) backbone linkage and wherein bold type represents the spacer sequence.

TABLE 37
Guide RNA sequence
gRNA sequence (5′-3′)

GCCAUCCUGCCAAGAAUGAGGUUUUAGAGCUAGAAAUAGCAAGUUAAAAUAAGGCUAGUCC
GUUAUCAACUUGAAAAAGUGGCACCGAGUCGGUGCUUUU (SEQ ID NO: 477)
GCCAUCCUGCCAAGAACGAGGUUUUAGAGCUAGAAAUAGCAAGUUAAAAUAAGGCUAGUCC
GUUAUCAACUUGAAAAAGUGGCACCGAGUCGGUGCUUUU (SEQ ID NO: 1044)
GCAACUUACCCAGAGGCAAAGUUUUAGAGCUAGAAAUAGCAAGUUAAAAUAAGGCUAGUCC
GUUAUCAACUUGAAAAAGUGGCACCGAGUCGGUGCUUUU (SEQ ID NO: 499)
UAUAGGAAAACCAGUGAGUCGUUUUAGAGCUAGAAAUAGCAAGUUAAAAUAAGGCUAGUCC
GUUAUCAACUUGAAAAAGUGGCACCGAGUCGGUGCUUUU (SEQ ID NO: 497)
UACUCACCUCUGCAUGCUCAGUUUUAGAGCUAGAAAUAGCAAGUUAAAAUAAGGCUAGUCC
GUUAUCAACUUGAAAAAGUGGCACCGAGUCGGUGCUUUU (SEQ ID NO: 480)
GCCAUCCUGCCAAGAACGAGGUUUUAGAGCUAGAAAUAGCAAGUUAAAAUAAGGCUAGUCC
GUUAUCAACUUGAAAAAGUGGCACCGAGUCGGUGCUUUU (SEQ ID NO: 1044)

### OTHER EMBODIMENTS

From the foregoing description, it will be apparent that variations and modifications may be made to the aspects or embodiments described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference. The disclosure may be related to International Patent Applications No. PCT/US2022/030359, filed May 20, 2022, PCT/US2022/029278, filed May 13, 2022, and PCT/US23/79329, filed Nov. 10, 2023, the disclosures of which are each incorporated herein by reference in their entireties for all purposes.

What is claimed:

1. A lipid nanoparticle (LNP) comprising a guide polynucleotide comprising a sequence selected from any one or more of the following:

| | | SEQ ID |
|---|---|---|
| gRNA ID | SEQUENCE | NO |
| gRNA_361 | UAUAGGAAAACCAGTGAGTCGUUUUAGAGCUAGAAAUAGCAAGUUAAA | 479 |
| | AUAAGGCUAGUCCGUUAUCAACUUGAAAAAGUGGCACCGAGUCGGUGC | |
| | UUUU | |
| gRNA_362 | UACUCACCUCUGCAUGCUCAGUUUUAGAGCUAGAAAUAGCAAGUUAAA | 480 |
| | AUAAGGCUAGUCCGUUAUCAACUUGAAAAAGUGGCACCGAGUCGGUGC | |
| | UUUU | |
| gRNA_363 | ACUCACCUCUGCAUGCUCAUGUUUUAGAGCUAGAAAUAGCAAGUUAAA | 481 |
| | AUAAGGCUAGUCCGUUAUCAACUUGAAAAAGUGGCACCGAGUCGGUGC | |
| | UUUU | |
| gRNA_364 | UACCACCUAUGAGAGAAGACGUUUUAGAGCUAGAAAUAGCAAGUUAAA | 482 |
| | AUAAGGCUAGUCCGUUAUCAACUUGAAAAAGUGGCACCGAGUCGGUGC | |
| | UUUU | |
| gRNA_365 | AUACUCACCUCUGCAUGCUCAGUUUUAGUACUCUGUAAUGAAAAUUAC | 483 |
| | AGAAUCUACUAAAACAAGGCAAAAUGCCGUGUUUAUCUCGUCAACUUG | |
| | UUGGCGAGAUUUU | |
| gRNA_366 | ACUGGUUUUCCUAUAAGGUGUGUUUUAGUACUCUGUAAUGAAAAUUAC | 484 |
| | AGAAUCUACUAAAACAAGGCAAAAUGCCGUGUUUAUCUCGUCAACUUG | |
| | UUGGCGAGAUUUU | |
| gRNA_367 | GUUCUGUCUUUUGGUCAGGACAACCGUCUAGCUAUAAGUGCUGCAGGG | 485 |
| | UGUGAGAAACUCCUAUUGCUGGACGAUGUCUCUUACGAGGCAUUAGCA | |
| | CUUGGCAGGAUGGCUUCUCAUCG | |
| gRNA_368 | GUUCUGUCUUUUGGUCAGGACAACCGUCUAGCUAUAAGUGCUGCAGGG | 486 |
| | UGUGAGAAACUCCUAUUGCUGGACGAUGUCUCUUACGAGGCAUUAGCA | |
| | CUCCUAUAAGGUGUGAAAGUCUG | |
| gRNA_369 | GUUCUGUCUUUUGGUCAGGACAACCGUCUAGCUAUAAGUGCUGCAGGG | 487 |
| | UGUGAGAAACUCCUAUUGCUGGACGAUGUCUCUUACGAGGCAUUAGCA | |
| | CUGAGCCCAUGCAGCUCUCCAGA | |
| gRNA_370 | GUUCUGUCUUUUGGUCAGGACAACCGUCUAGCUAUAAGUGCUGCAGGG | 488 |
| | UGUGAGAAACUCCUAUUGCUGGACGAUGUCUCUUACGAGGCAUUAGCA | |
| | CCUCCUCAGUUGUGAGCCCAUGC | |
| gRNA_371 | GUUCUGUCUUUUGGUCAGGACAACCGUCUAGCUAUAAGUGCUGCAGGG | 489 |
| | UGUGAGAAACUCCUAUUGCUGGACGAUGUCUCUUACGAGGCAUUAGCA | |
| | CGUAGAAGGGAUAUACAAAGUGG | |
| gRNA_372 | GUUCUGUCUUUUGGUCAGGACAACCGUCUAGCUAUAAGUGCUGCAGGG | 490 |
| | UGUGAGAAACUCCUAUUGCUGGACGAUGUCUCUUACGAGGCAUUAGCA | |
| | CCCACUUUGUAUAUCCCUUCUAC | |
| gRNA_373 | GUUCUGUCUUUUGGUCAGGACAACCGUCUAGCUAUAAGUGCUGCAGGG | 491 |
| | UGUGAGAAACUCCUAUUGCUGGACGAUGUCUCUUACGAGGCAUUAGCA | |
| | CGGUGUCUAUUUCCACUUUGUAU | |
| gRNA_374 | GUUCUGUCUUUUGGUCAGGACAACCGUCUAGCUAUAAGUGCUGCAGGG | 492 |
| | UGUGAGAAACUCCUAUUGCUGGACGAUGUCUCUUACGAGGCAUUAGCA | |
| | CCAUGAGCAUGCAGAGGUGAGUA | |
| gRNA1594 | CAACUUACCCAGAGGCAAAUGUUUUAGAGCUAGAAAUAGCAAGUUAAA | 493 |
| | AUAAGGCUAGUCCGUUAUCAACUUGAAAAAGUGGCACCGAGUCGGUGC | |
| | UUUU | |
| gRNA1595 | AAUGGCUCCCAGGUGUCAUCGUUUUAGAGCUAGAAAUAGCAAGUUAAA | 494 |
| | AUAAGGCUAGUCCGUUAUCAACUUGAAAAAGUGGCACCGAGUCGGUGC | |
| | UUUU | |
| gRNA1596 | GGCUCCCAGGUGUCAUCAGCGUUUUAGAGCUAGAAAUAGCAAGUUAAA | 495 |
| | AUAAGGCUAGUCCGUUAUCAACUUGAAAAAGUGGCACCGAGUCGGUGC | |
| | UUUU | |
| gRNA1597 | CUCUCAUAGGUGGUAUUCACGUUUUAGAGCUAGAAAUAGCAAGUUAAA | 496 |
| | AUAAGGCUAGUCCGUUAUCAACUUGAAAAAGUGGCACCGAGUCGGUGC | |
| | UUUU | |
| gRNA1598 | UAUAGGAAAACCAGUGAGUCGUUUUAGAGCUAGAAAUAGCAAGUUAAA | 497 |
| | AUAAGGCUAGUCCGUUAUCAACUUGAAAAAGUGGCACCGAGUCGGUGC | |
| | UUUU | |
| gRNA1599 | UACUCACCUCUGCAUGCUCAGUUUUAGAGCUAGAAAUAGCAAGUUAAA | 480 |
| | AUAAGGCUAGUCCGUUAUCAACUUGAAAAAGUGGCACCGAGUCGGUGC | |
| | UUUU | |
| gRNA1600 | GCAACUUACCCAGAGGCAAAGUUUUAGAGCUAGAAAUAGCAAGUUAAA | 499 |
| | AUAAGGCUAGUCCGUUAUCAACUUGAAAAAGUGGCACCGAGUCGGUGC | |
| | UUUU | |
| gRNA1601 | UCUGUAUACUCACCUCUGCAGUUUUAGAGCUAGAAAUAGCAAGUUAAA | 500 |
| | AUAAGGCUAGUCCGUUAUCAACUUGAAAAAGUGGCACCGAGUCGGUGC | |
| | UUUU | |
| gRNA1602 | GAAACACUCACCGUAGGGCCGUUUUAGAGCUAGAAAUAGCAAGUUAAA | 501 |
| | AUAAGGCUAGUCCGUUAUCAACUUGAAAAAGUGGCACCGAGUCGGUGC | |
| | UUUU | |
| gRNA1603 | CUCUACACCCAGGGCACCGGGUUUUAGAGCUAGAAAUAGCAAGUUAAA | 502 |
| | AUAAGGCUAGUCCGUUAUCAACUUGAAAAAGUGGCACCGAGUCGGUGC | |
| | UUUU | |
| gRNA1604 | ACACCUUAUAGGAAAACCAGGUUUUAGAGCUAGAAAUAGCAAGUUAAA | 503 |
| | AUAAGGCUAGUCCGUUAUCAACUUGAAAAAGUGGCACCGAGUCGGUGC | |
| | UUUU | |
| gRNA1605 | AUAGGAAAACCAGUGAGUCUGUUUUAGAGCUAGAAAUAGCAAGUUAAA | 504 |
| | AUAAGGCUAGUCCGUUAUCAACUUGAAAAAGUGGCACCGAGUCGGUGC | |
| | UUUU | |
| gRNA1606 | ACUCACCUCUGCAUGCUCAUGUUUUAGAGCUAGAAAUAGCAAGUUAAA | 481 |
| | AUAAGGCUAGUCCGUUAUCAACUUGAAAAAGUGGCACCGAGUCGGUGC | |
| | UUUU | |
| gRNA1607 | CUCACCGUAGGGCCAGCCUCGUUUUAGAGCUAGAAAUAGCAAGUUAAA | 506 |
| | AUAAGGCUAGUCCGUUAUCAACUUGAAAAAGUGGCACCGAGUCGGUGC | |
| | UUUU | |
| gRNA1746 | AACCUGCUGAUUCUGAUUAUGUUUUAGAGCUAGAAAUAGCAAGUUAAA | 507 |
| | AUAAGGCUAGUCCGUUAUCAACUUGAAAAAGUGGCACCGAGUCGGUGC | |
| | UUUU | |
| gRNA1747 | AAGAGAGAAUAAGUAACCCAUGUUUUAGUACUCUGUAAUGAAAAUUAC | 508 |
| | AGAAUCUACUAAAACAAGGCAAAAUGCCGUGUUUAUCUCGUCAACUUG | |
| | UUGGCGAGAUUUU | |
| gRNA1748 | AAGCAGCCUAGCUCAGGAGAAGUUUUAGUACUCUGUAAUGAAAAUUAC | 509 |
| | AGAAUCUACUAAAACAAGGCAAAAUGCCGUGUUUAUCUCGUCAACUUG | |
| | UUGGCGAGAUUUU | |
| gRNA1749 | AAGUCCACUCAUUCUUGGCAGUUUUAGAGCUAGAAAUAGCAAGUUAAA | 510 |
| | AUAAGGCUAGUCCGUUAUCAACUUGAAAAAGUGGCACCGAGUCGGUGC | |
| | UUUU | |
| gRNA1750 | ACGAUGAGAAGCCAUCCUGCCGUUUUAGUACUCUGUAAUGAAAAUUAC | 511 |
| | AGAAUCUACUAAAACAAGGCAAAAUGCCGUGUUUAUCUCGUCAACUUG | |
| | UUGGCGAGAUUUU | |
| gRNA1751 | AGACAAGGUUCAUAUUUGUAGUUUUAGAGCUAGAAAUAGCAAGUUAAA | 512 |
| | AUAAGGCUAGUCCGUUAUCAACUUGAAAAAGUGGCACCGAGUCGGUGC | |
| | UUUU | |
| gRNA1752 | AGGCUGGGAGCAGCCAUCACGUUUUAGAGCUAGAAAUAGCAAGUUAAA | 513 |
| | AUAAGGCUAGUCCGUUAUCAACUUGAAAAAGUGGCACCGAGUCGGUGC | |
| | UUUU | |
| gRNA1753 | AUAAGUAACCCAUACAAAUAGUUUUAGAGCUAGAAAUAGCAAGUUAAA | 514 |
| | AUAAGGCUAGUCCGUUAUCAACUUGAAAAAGUGGCACCGAGUCGGUGC | |
| | UUUU | |
| gRNA1754 | AUACUCACUUCUCCUGAGCUGUUUUAGAGCUAGAAAUAGCAAGUUAAA | 515 |
| | AUAAGGCUAGUCCGUUAUCAACUUGAAAAAGUGGCACCGAGUCGGUGC | |
| | UUUU | |
| gRNA1755 | AUUAUUGACUUAGUCAACAAGUUUUAGAGCUAGAAAUAGCAAGUUAAA | 516 |
| | AUAAGGCUAGUCCGUUAUCAACUUGAAAAAGUGGCACCGAGUCGGUGC | |
| | UUUU | |
| gRNA1756 | CAAAUAUGAACCUUGUCUAGGUUUUAGAGCUAGAAAUAGCAAGUUAAA | 517 |
| | AUAAGGCUAGUCCGUUAUCAACUUGAAAAAGUGGCACCGAGUCGGUGC | |
| | UUUU | |
| gRNA1757 | CAGAAGUCCACUCAUUCUUGGGUUUUAGUACUCUGUAAUGAAAAUUAC | 518 |
| | AGAAUCUACUAAAACAAGGCAAAAUGCCGUGUUUAUCUCGUCAACUUG | |
| | UUGGCGAGAUUUU | |
| gRNA1758 | CAGGCUGGGAGCAGCCAUCACGUUUUAGUACUCUGUAAUGAAAAUUAC | 519 |
| | AGAAUCUACUAAAACAAGGCAAAAUGCCGUGUUUAUCUCGUCAACUUG | |
| | UUGGCGAGAUUUU | |
| gRNA1759 | CCAUCCUGCCAAGAAUGAGUGUUUUAGAGCUAGAAAUAGCAAGUUAAA | 520 |
| | AUAAGGCUAGUCCGUUAUCAACUUGAAAAAGUGGCACCGAGUCGGUGC | |
| | UUUU | |
| gRNA1760 | CCUGCUGAUUCUGAUUAUUGAGUUUUAGUACUCUGUAAUGAAAAUUAC | 521 |
| | AGAAUCUACUAAAACAAGGCAAAAUGCCGUGUUUAUCUCGUCAACUUG | |
| | UUGGCGAGAUUUU | |
| gRNA1761 | CGAUGCUCUAAUCUCUCUAGAGUUUUAGUACUCUGUAAUGAAAAUUAC | 522 |
| | AGAAUCUACUAAAACAAGGCAAAAUGCCGUGUUUAUCUCGUCAACUUG | |
| | UUGGCGAGAUUUU | |
| gRNA1762 | CUAAGUCAAUAAUCAGAAUCAGUUUUAGUACUCUGUAAUGAAAAUUAC | 523 |
| | AGAAUCUACUAAAACAAGGCAAAAUGCCGUGUUUAUCUCGUCAACUUG | |
| | UUGGCGAGAUUUU | |
| gRNA1763 | CUAGACAAGGUUCAUAUUUGUGUUUUAGUACUCUGUAAUGAAAAUUAC | 524 |
| | AGAAUCUACUAAAACAAGGCAAAAUGCCGUGUUUAUCUCGUCAACUUG | |
| | UUGGCGAGAUUUU | |
| gRNA1764 | GAACCUUGUCUAGAGAGAUUGUUUUAGAGCUAGAAAUAGCAAGUUAAA | 525 |
| | AUAAGGCUAGUCCGUUAUCAACUUGAAAAAGUGGCACCGAGUCGGUGC | |
| | UUUU | |
| gRNA1765 | GAAGUCCACUCAUUCUUGGCGUUUUAGAGCUAGAAAUAGCAAGUUAAA | 526 |
| | AUAAGGCUAGUCCGUUAUCAACUUGAAAAAGUGGCACCGAGUCGGUGC | |
| | UUUU | |
| gRNA1766 | GAAUCAGCAGGUUUGCAGUCGUUUUAGAGCUAGAAAUAGCAAGUUAAA | 527 |
| | AUAAGGCUAGUCCGUUAUCAACUUGAAAAAGUGGCACCGAGUCGGUGC | |
| | UUUU | |
| gRNA1767 | GAAUGAGUGGACUUCUGUGAGUUUUAGAGCUAGAAAUAGCAAGUUAAA | 528 |
| | AUAAGGCUAGUCCGUUAUCAACUUGAAAAAGUGGCACCGAGUCGGUGC | |
| | UUUU | |
| gRNA1768 | GACUGCAAACCUGCUGAUUCGUUUUAGAGCUAGAAAUAGCAAGUUAAA | 529 |
| | AUAAGGCUAGUCCGUUAUCAACUUGAAAAAGUGGCACCGAGUCGGUGC | |
| | UUUU | |
| gRNA1769 | GACUUAGUCAACAAAGAGAGAGUUUUAGUACUCUGUAAUGAAAAUUAC | 530 |
| | AGAAUCUACUAAAACAAGGCAAAAUGCCGUGUUUAUCUCGUCAACUUG | |
| | UUGGCGAGAUUUU | |
| gRNA1770 | GAUAAGCAGCCUAGCUCAGGGUUUUAGAGCUAGAAAUAGCAAGUUAAA | 531 |
| | AUAAGGCUAGUCCGUUAUCAACUUGAAAAAGUGGCACCGAGUCGGUGC | |
| | UUUU | |
| gRNA1771 | GAUGAGAAGCCAUCCUGCCAGUUUUAGAGCUAGAAAUAGCAAGUUAAA | 532 |
| | AUAAGGCUAGUCCGUUAUCAACUUGAAAAAGUGGCACCGAGUCGGUGC | |
| | UUUU | |
| gRNA1773 | GCUUUUAUACUCACUUCUCCGUUUUAGAGCUAGAAAUAGCAAGUUAAA | 534 |
| | AUAAGGCUAGUCCGUUAUCAACUUGAAAAAGUGGCACCGAGUCGGUGC | |
| | UUUU | |
| gRNA1774 | GGAUAAGCAGCCUAGCUCAGGGUUUUAGUACUCUGUAAUGAAAAUUAC | 535 |
| | AGAAUCUACUAAAACAAGGCAAAAUGCCGUGUUUAUCUCGUCAACUUG | |
| | UUGGCGAGAUUUU | |
| gRNA1775 | GUCUAGAGAGAUUAGAGCAUGUUUUAGAGCUAGAAAUAGCAAGUUAAA | 536 |
| | AUAAGGCUAGUCCGUUAUCAACUUGAAAAAGUGGCACCGAGUCGGUGC | |
| | UUUU | |
| gRNA1776 | GUGAUGGCUGCUCCCAGCCUGUUUUAGAGCUAGAAAUAGCAAGUUAAA | 537 |
| | AUAAGGCUAGUCCGUUAUCAACUUGAAAAAGUGGCACCGAGUCGGUGC | |
| | UUUU | |
| gRNA1777 | UACUUAUUCUCUCUUUGUUGAGUUUUAGUACUCUGUAAUGAAAAUUAC | 538 |
| | AGAAUCUACUAAAACAAGGCAAAAUGCCGUGUUUAUCUCGUCAACUUG | |
| | UUGGCGAGAUUUU | |
| gRNA1778 | UAUUCUCUCUUUGUUGACUAAGUUUUAGUACUCUGUAAUGAAAAUUAC | 539 |
| | AGAAUCUACUAAAACAAGGCAAAAUGCCGUGUUUAUCUCGUCAACUUG | |
| | UUGGCGAGAUUUU | |
| gRNA1779 | UAUUGACUUAGUCAACAAAGGUUUUAGAGCUAGAAAUAGCAAGUUAAA | 540 |
| | AUAAGGCUAGUCCGUUAUCAACUUGAAAAAGUGGCACCGAGUCGGUGC | |
| | UUUU | |
| gRNA1780 | UAUUGACUUAGUCAACAAAGAGUUUUAGUACUCUGUAAUGAAAAUUAC | 541 |
| | AGAAUCUACUAAAACAAGGCAAAAUGCCGUGUUUAUCUCGUCAACUUG | |
| | UUGGCGAGAUUUU | |
| gRNA1781 | UCAGAAUCAGCAGGUUUGCAGGUUUUAGUACUCUGUAAUGAAAAUUAC | 542 |
| | AGAAUCUACUAAAACAAGGCAAAAUGCCGUGUUUAUCUCGUCAACUUG | |
| | UUGGCGAGAUUUU | |
| gRNA1782 | UCCACUCAUUCUUGGCAGGAGUUUUAGAGCUAGAAAUAGCAAGUUAAA | 543 |
| | AUAAGGCUAGUCCGUUAUCAACUUGAAAAAGUGGCACCGAGUCGGUGC | |
| | UUUU | |
| gRNA1783 | UCUCUCUUUGUUGACUAAGUCGUUUUAGUACUCUGUAAUGAAAAUUAC | 544 |
| | AGAAUCUACUAAAACAAGGCAAAAUGCCGUGUUUAUCUCGUCAACUUG | |
| | UUGGCGAGAUUUU | |
| gRNA1784 | UGAGAAGCCAUCCUGCCAAGAGUUUUAGUACUCUGUAAUGAAAAUUAC | 545 |
| | AGAAUCUACUAAAACAAGGCAAAAUGCCGUGUUUAUCUCGUCAACUUG | |
| | UUGGCGAGAUUUU | |
| gRNA1785 | UGAGCUAGGCUGCUUAUCCCUGUUUUAGUACUCUGUAAUGAAAAUUAC | 546 |
| | AGAAUCUACUAAAACAAGGCAAAAUGCCGUGUUUAUCUCGUCAACUUG | |
| | UUGGCGAGAUUUU | |
| gRNA1786 | UGAGUAUAAAAGCCCCAGGCGUUUUAGAGCUAGAAAUAGCAAGUUAAA | 547 |
| | AUAAGGCUAGUCCGUUAUCAACUUGAAAAAGUGGCACCGAGUCGGUGC | |
| | UUUU | |
| gRNA1787 | UGAUGGCUGCUCCCAGCCUGGUUUUAGAGCUAGAAAUAGCAAGUUAAA | 548 |
| | AUAAGGCUAGUCCGUUAUCAACUUGAAAAAGUGGCACCGAGUCGGUGC | |
| | UUUU | |
| gRNA1788 | UGCCAAGAAUGAGUGGACUUCGUUUUAGUACUCUGUAAUGAAAAUUAC | 549 |
| | AGAAUCUACUAAAACAAGGCAAAAUGCCGUGUUUAUCUCGUCAACUUG | |
| | UUGGCGAGAUUUU | |
| gRNA1789 | UGCCAAUCUGACUGCAAACCUGUUUUAGUACUCUGUAAUGAAAAUUAC | 550 |
| | AGAAUCUACUAAAACAAGGCAAAAUGCCGUGUUUAUCUCGUCAACUUG | |
| | UUGGCGAGAUUUU | |
| gRNA1790 | UGUUGACUAAGUCAAUAAUCGUUUUAGAGCUAGAAAUAGCAAGUUAAA | 551 |
| | AUAAGGCUAGUCCGUUAUCAACUUGAAAAAGUGGCACCGAGUCGGUGC | |
| | UUUU | |
| gRNA1791 | UUGACUUAGUCAACAAAGAGGUUUUAGAGCUAGAAAUAGCAAGUUAAA | 552 |
| | AUAAGGCUAGUCCGUUAUCAACUUGAAAAAGUGGCACCGAGUCGGUGC | |
| | UUUU | |
| gRNA1792 | UUUGUUGACUAAGUCAAUAAUGUUUUAGUACUCUGUAAUGAAAAUUAC | 553 |
| | AGAAUCUACUAAAACAAGGCAAAAUGCCGUGUUUAUCUCGUCAACUUG | |
| | UUGGCGAGAUUUU | |
| gRNA-#1 | AAAAGCCCCAGGCUGGGAGCGUUUUAGAGCUAGAAAUAGCAAGUUAAA | 554 |
| | AUAAGGCUAGUCCGUUAUCAACUUGAAAAAGUGGCACCGAGUCGGUGC | |
| | UUUU | |
| gRNA-#2 | AAGUGAGUAUAAAAGCCCCAGUUUUAGAGCUAGAAAUAGCAAGUUAAA | 555 |
| | AUAAGGCUAGUCCGUUAUCAACUUGAAAAAGUGGCACCGAGUCGGUGC | |
| | UUUU | |
| gRNA-#3 | AAUAAUCAGAAUCAGCAGGUUGUUUUAGUACUCUGUAAUGAAAAUUAC | 556 |
| | AGAAUCUACUAAAACAAGGCAAAAUGCCGUGUUUAUCUCGUCAACUUG | |
| | UUGGCGAGAUUUU | |
| gRNA-#4 | AAUAUGAACCUUGUCUAGAGGUUUUAGAGCUAGAAAUAGCAAGUUAAA | 557 |
| | AUAAGGCUAGUCCGUUAUCAACUUGAAAAAGUGGCACCGAGUCGGUGC | |
| | UUUU | |
| gRNA-#5 | AAUGAGUGGACUUCUGUGAUGUUUUAGAGCUAGAAAUAGCAAGUUAAA | 558 |
| | AUAAGGCUAGUCCGUUAUCAACUUGAAAAAGUGGCACCGAGUCGGUGC | |
| | UUUU | |
| gRNA-#6 | ACAAAUAUGAACCUUGUCUAGGUUUUAGUACUCUGUAAUGAAAAUUAC | 559 |
| | AGAAUCUACUAAAACAAGGCAAAAUGCCGUGUUUAUCUCGUCAACUUG | |
| | UUGGCGAGAUUUU | |
| gRNA-#7 | ACAGAAGUCCACUCAUUCUUGUUUUAGAGCUAGAAAUAGCAAGUUAAA | 560 |
| | AUAAGGCUAGUCCGUUAUCAACUUGAAAAAGUGGCACCGAGUCGGUGC | |
| | UUUU | |
| gRNA-#8 | ACCUUGUCUAGAGAGAUUAGGUUUUAGAGCUAGAAAUAGCAAGUUAAA | 561 |
| | AUAAGGCUAGUCCGUUAUCAACUUGAAAAAGUGGCACCGAGUCGGUGC | |
| | UUUU | |
| gRNA-#9 | AGAAGCCAUCCUGCCAAGAAGUUUUAGAGCUAGAAAUAGCAAGUUAAA | 562 |
| | AUAAGGCUAGUCCGUUAUCAACUUGAAAAAGUGGCACCGAGUCGGUGC | |
| | UUUU | |
| gRNA-#10 | AGCAGGUUUGCAGUCAGAUUGUUUUAGAGCUAGAAAUAGCAAGUUAAA | 563 |
| | AUAAGGCUAGUCCGUUAUCAACUUGAAAAAGUGGCACCGAGUCGGUGC | |
| | UUUU | |
| gRNA-#11 | AGGGAUAAGCAGCCUAGCUCGUUUUAGAGCUAGAAAUAGCAAGUUAAA | 564 |
| | AUAAGGCUAGUCCGUUAUCAACUUGAAAAAGUGGCACCGAGUCGGUGC | |
| | UUUU | |
| gRNA-#12 | AGGUUUGCAGUCAGAUUGGCGUUUUAGAGCUAGAAAUAGCAAGUUAAA | 565 |
| | AUAAGGCUAGUCCGUUAUCAACUUGAAAAAGUGGCACCGAGUCGGUGC | |
| | UUUU | |
| gRNA-#13 | AGUAUAAAAGCCCCAGGCUGGUUUUAGAGCUAGAAAUAGCAAGUUAAA | 566 |
| | AUAAGGCUAGUCCGUUAUCAACUUGAAAAAGUGGCACCGAGUCGGUGC | |
| | UUUU | |
| gRNA-#14 | AGUCAAUAAUCAGAAUCAGCGUUUUAGAGCUAGAAAUAGCAAGUUAAA | 567 |
| | AUAAGGCUAGUCCGUUAUCAACUUGAAAAAGUGGCACCGAGUCGGUGC | |
| | UUUU | |
| gRNA-#15 | AUAAUCAGAAUCAGCAGGUUGUUUUAGAGCUAGAAAUAGCAAGUUAAA | 568 |
| | AUAAGGCUAGUCCGUUAUCAACUUGAAAAAGUGGCACCGAGUCGGUGC | |
| | UUUU | |
| gRNA-#16 | GUUCUGUCUUUUGGUCAGGACAACCGUCUAGCUAUAAGUGCUGCAGGG | 569 |
| | UGUGAGAAACUCCUAUUGCUGGACGAUGUCUCUUACGAGGCAUUAGCA | |
| | CUUGACUUAGUCAACAAAGAGAG | |
| gRNA-#17 | GUUCUGUCUUUUGGUCAGGACAACCGUCUAGCUAUAAGUGCUGCAGGG | 570 |
| | UGUGAGAAACUCCUAUUGCUGGACGAUGUCUCUUACGAGGCAUUAGCA | |
| | CUCUCUUUGUUGACUAAGUCAAU | |
| gRNA-#18 | GUUCUGUCUUUUGGUCAGGACAACCGUCUAGCUAUAAGUGCUGCAGGG | 571 |
| | UGUGAGAAACUCCUAUUGCUGGACGAUGUCUCUUACGAGGCAUUAGCA | |
| | CUGAUUAUUGACUUAGUCAACAA | |
| gRNA-#19 | GUUCUGUCUUUUGGUCAGGACAACCGUCUAGCUAUAAGUGCUGCAGGG | 485 |
| | UGUGAGAAACUCCUAUUGCUGGACGAUGUCUCUUACGAGGCAUUAGCA | |
| | CUUGGCAGGAUGGCUUCUCAUCG | |
| gRNA-#20 | GUUCUGUCUUUUGGUCAGGACAACCGUCUAGCUAUAAGUGCUGCAGGG | 573 |
| | UGUGAGAAACUCCUAUUGCUGGACGAUGUCUCUUACGAGGCAUUAGCA | |
| | CACUUAGUCAACAAAGAGAGAAU | |
| gRNA-#21 | GUUCUGUCUUUUGGUCAGGACAACCGUCUAGCUAUAAGUGCUGCAGGG | 574 |
| | UGUGAGAAACUCCUAUUGCUGGACGAUGUCUCUUACGAGGCAUUAGCA | |
| | CGCAGGGAUAAGCAGCCUAGCUC | |
| gRNA-#22 | GUUCUGUCUUUUGGUCAGGACAACCGUCUAGCUAUAAGUGCUGCAGGG | 575 |
| | UGUGAGAAACUCCUAUUGCUGGACGAUGUCUCUUACGAGGCAUUAGCA | |
| | CGUAUGGGUUACUUAUUCUCUCU | |
| gRNA-#23 | CAAGAAUGAGUGGACUUCUGGUUUUAGAGCUAGAAAUAGCAAGUUAAA | 576 |
| | AUAAGGCUAGUCCGUUAUCAACUUGAAAAAGUGGCACCGAGUCGGUGC | |
| | UUUU | |
| gRNA-#24 | CAAUCUGACUGCAAACCUGCGUUUUAGAGCUAGAAAUAGCAAGUUAAA | 577 |
| | AUAAGGCUAGUCCGUUAUCAACUUGAAAAAGUGGCACCGAGUCGGUGC | |
| | UUUU | |
| gRNA-#25 | CACAGAAGUCCACUCAUUCUGUUUUAGAGCUAGAAAUAGCAAGUUAAA | 578 |
| | AUAAGGCUAGUCCGUUAUCAACUUGAAAAAGUGGCACCGAGUCGGUGC | |
| | UUUU | |
| gRNA-#26 | CAGACGAUGAGAAGCCAUCCGUUUUAGAGCUAGAAAUAGCAAGUUAAA | 579 |
| | AUAAGGCUAGUCCGUUAUCAACUUGAAAAAGUGGCACCGAGUCGGUGC | |
| | UUUU | |
| gRNA-#27 | CAGCAGGUUUGCAGUCAGAUGUUUUAGAGCUAGAAAUAGCAAGUUAAA | 580 |
| | AUAAGGCUAGUCCGUUAUCAACUUGAAAAAGUGGCACCGAGUCGGUGC | |
| | UUUU | |
| gRNA-#28 | CAGGAUGGCUUCUCAUCGUCGUUUUAGAGCUAGAAAUAGCAAGUUAAA | 581 |
| | AUAAGGCUAGUCCGUUAUCAACUUGAAAAAGUGGCACCGAGUCGGUGC | |
| | UUUU | |
| gRNA-#29 | CAGGUUUGCAGUCAGAUUGGCGUUUUAGUACUCUGUAAUGAAAAUUAC | 582 |
| | AGAAUCUACUAAAACAAGGCAAAAUGCCGUGUUUAUCUCGUCAACUUG | |
| | UUGGCGAGAUUUU | |
| gRNA-#30 | CAGUCAGAUUGGCAGGGAUAGUUUUAGAGCUAGAAAUAGCAAGUUAAA | 583 |
| | AUAAGGCUAGUCCGUUAUCAACUUGAAAAAGUGGCACCGAGUCGGUGC | |
| | UUUU | |
| gRNA-#31 | CCACUCAUUCUUGGCAGGAUGUUUUAGAGCUAGAAAUAGCAAGUUAAA | 584 |
| | AUAAGGCUAGUCCGUUAUCAACUUGAAAAAGUGGCACCGAGUCGGUGC | |
| | UUUU | |
| gRNA-#32 | CUAAGUCAAUAAUCAGAAUCGUUUUAGAGCUAGAAAUAGCAAGUUAAA | 585 |
| | AUAAGGCUAGUCCGUUAUCAACUUGAAAAAGUGGCACCGAGUCGGUGC | |
| | UUUU | |
| gRNA-#33 | GUUCUGUCUUUUGGUCAGGACAACCGUCUAGCUAUAAGUGCUGCAGGG | 586 |
| | UGUGAGAAACUCCUAUUGCUGGACGAUGUCUCUUACGAGGCAUUAGCA | |
| | CGUCAACAAAGAGAGAAUAAGUA | |
| gRNA-#34 | CUUAUCCCUGCCAAUCUGACGUUUUAGAGCUAGAAAUAGCAAGUUAAA | 587 |
| | AUAAGGCUAGUCCGUUAUCAACUUGAAAAAGUGGCACCGAGUCGGUGC | |
| | UUUU | |
| gRNA-#35 | GUUCUGUCUUUUGGUCAGGACAACCGUCUAGCUAUAAGUGCUGCAGGG | 588 |
| | UGUGAGAAACUCCUAUUGCUGGACGAUGUCUCUUACGAGGCAUUAGCA | |
| | CUCCCUGCCAAUCUGACUGCAAA | |
| gRNA-#36 | GUUCUGUCUUUUGGUCAGGACAACCGUCUAGCUAUAAGUGCUGCAGGG | 589 |
| | UGUGAGAAACUCCUAUUGCUGGACGAUGUCUCUUACGAGGCAUUAGCA | |
| | CUUCUCUCUUUGUUGACUAAGUC | |
| gRNA-#37 | GUUCUGUCUUUUGGUCAGGACAACCGUCUAGCUAUAAGUGCUGCAGGG | 590 |
| | UGUGAGAAACUCCUAUUGCUGGACGAUGUCUCUUACGAGGCAUUAGCA | |
| | CUCCUGAGCUAGGCUGCUUAUCC | |
| gRNA-#38 | CUUCUGUGAUGGCUGCUCCCGUUUUAGAGCUAGAAAUAGCAAGUUAAA | 591 |
| | AUAAGGCUAGUCCGUUAUCAACUUGAAAAAGUGGCACCGAGUCGGUGC | |
| | UUUU | |
| gRNA-#39 | GUUCUGUCUUUUGGUCAGGACAACCGUCUAGCUAUAAGUGCUGCAGGG | 592 |
| | UGUGAGAAACUCCUAUUGCUGGACGAUGUCUCUUACGAGGCAUUAGCA | |
| | CUGUGAUGGCUGCUCCCAGCCUG | |
| gRNA-#40 | GUUCUGUCUUUUGGUCAGGACAACCGUCUAGCUAUAAGUGCUGCAGGG | 593 |
| | UGUGAGAAACUCCUAUUGCUGGACGAUGUCUCUUACGAGGCAUUAGCA | |
| | CGCAGGAUGGCUUCUCAUCGUCU | |
| gRNA-#41 | GUUCUGUCUUUUGGUCAGGACAACCGUCUAGCUAUAAGUGCUGCAGGG | 594 |
| | UGUGAGAAACUCCUAUUGCUGGACGAUGUCUCUUACGAGGCAUUAGCA | |
| | CUCUAGAGAGAUUAGAGCAUCGG | |
| gRNA-#42 | GUUCUGUCUUUUGGUCAGGACAACCGUCUAGCUAUAAGUGCUGCAGGG | 595 |
| | UGUGAGAAACUCCUAUUGCUGGACGAUGUCUCUUACGAGGCAUUAGCA | |
| | CGUUGACUAAGUCAAUAAUCAGA | |
| gRNA-#43 | GUUCUGUCUUUUGGUCAGGACAACCGUCUAGCUAUAAGUGCUGCAGGG | 596 |
| | UGUGAGAAACUCCUAUUGCUGGACGAUGUCUCUUACGAGGCAUUAGCA | |
| | CUAUACUCACUUCUCCUGAGCUA | |
| gRNA-#44 | GAAGUGAGUAUAAAAGCCCCGUUUUAGAGCUAGAAAUAGCAAGUUAAA | 597 |
| | AUAAGGCUAGUCCGUUAUCAACUUGAAAAAGUGGCACCGAGUCGGUGC | |
| | UUUU | |
| gRNA-#45 | GACAAGGUUCAUAUUUGUAUGUUUUAGAGCUAGAAAUAGCAAGUUAAA | 598 |
| | AUAAGGCUAGUCCGUUAUCAACUUGAAAAAGUGGCACCGAGUCGGUGC | |
| | UUUU | |
| gRNA-#46 | GAGUAUAAAAGCCCCAGGCUGUUUUAGAGCUAGAAAUAGCAAGUUAAA | 599 |
| | AUAAGGCUAGUCCGUUAUCAACUUGAAAAAGUGGCACCGAGUCGGUGC | |
| | UUUU | |
| gRNA-#47 | GAGUGGACUUCUGUGAUGGCGUUUUAGAGCUAGAAAUAGCAAGUUAAA | 600 |
| | AUAAGGCUAGUCCGUUAUCAACUUGAAAAAGUGGCACCGAGUCGGUGC | |
| | UUUU | |
| gRNA-#48 | GAUGGCUGCUCCCAGCCUGGGUUUUAGAGCUAGAAAUAGCAAGUUAAA | 601 |
| | AUAAGGCUAGUCCGUUAUCAACUUGAAAAAGUGGCACCGAGUCGGUGC | |
| | UUUU | |
| gRNA-#49 | GCAGCCUAGCUCAGGAGAAGGUUUUAGAGCUAGAAAUAGCAAGUUAAA | 602 |
| | AUAAGGCUAGUCCGUUAUCAACUUGAAAAAGUGGCACCGAGUCGGUGC | |
| | UUUU | |
| gRNA-#50 | GCUGCUUAUCCCUGCCAAUCGUUUUAGAGCUAGAAAUAGCAAGUUAAA | 603 |
| | AUAAGGCUAGUCCGUUAUCAACUUGAAAAAGUGGCACCGAGUCGGUGC | |
| | UUUU | |
| gRNA-#51 | GGGAUAAGCAGCCUAGCUCAGUUUUAGAGCUAGAAAUAGCAAGUUAAA | 604 |
| | AUAAGGCUAGUCCGUUAUCAACUUGAAAAAGUGGCACCGAGUCGGUGC | |
| | UUUU | |
| gRNA-#52 | GGUUUGCAGUCAGAUUGGCAGUUUUAGAGCUAGAAAUAGCAAGUUAAA | 605 |
| | AUAAGGCUAGUCCGUUAUCAACUUGAAAAAGUGGCACCGAGUCGGUGC | |
| | UUUU | |
| gRNA-#53 | GUUACUUAUUCUCUCUUUGUGUUUUAGAGCUAGAAAUAGCAAGUUAAA | 606 |
| | AUAAGGCUAGUCCGUUAUCAACUUGAAAAAGUGGCACCGAGUCGGUGC | |
| | UUUU | |
| gRNA-#54 | GUUCUGUCUUUUGGUCAGGACAACCGUCUAGCUAUAAGUGCUGCAGGG | 607 |
| | UGUGAGAAACUCCUAUUGCUGGACGAUGUCUCUUACGAGGCAUUAGCA | |
| | CCUUAUUCUCUCUUUGUUGACUA | |
| gRNA-#55 | GUUCUGUCUUUUGGUCAGGACAACCGUCUAGCUAUAAGUGCUGCAGGG | 608 |
| | UGUGAGAAACUCCUAUUGCUGGACGAUGUCUCUUACGAGGCAUUAGCA | |
| | CAUAUUUGUAUGGGUUACUUAUU | |
| gRNA-#56 | GUUCUGUCUUUUGGUCAGGACAACCGUCUAGCUAUAAGUGCUGCAGGG | 609 |
| | UGUGAGAAACUCCUAUUGCUGGACGAUGUCUCUUACGAGGCAUUAGCA | |
| | CACUAAGUCAAUAAUCAGAAUCA | |
| gRNA-#57 | GUUUGCAGUCAGAUUGGCAGGUUUUAGAGCUAGAAAUAGCAAGUUAAA | 610 |
| | AUAAGGCUAGUCCGUUAUCAACUUGAAAAAGUGGCACCGAGUCGGUGC | |
| | UUUU | |
| gRNA-#58 | GUUCUGUCUUUUGGUCAGGACAACCGUCUAGCUAUAAGUGCUGCAGGG | 611 |
| | UGUGAGAAACUCCUAUUGCUGGACGAUGUCUCUUACGAGGCAUUAGCA | |
| | CGCAGUCAGAUUGGCAGGGAUAA | |
| gRNA-#59 | UACAAAUAUGAACCUUGUCUGUUUUAGAGCUAGAAAUAGCAAGUUAAA | 612 |
| | AUAAGGCUAGUCCGUUAUCAACUUGAAAAAGUGGCACCGAGUCGGUGC | |
| | UUUU | |
| gRNA-#60 | UACUCACUUCUCCUGAGCUAGUUUUAGAGCUAGAAAUAGCAAGUUAAA | 613 |
| | AUAAGGCUAGUCCGUUAUCAACUUGAAAAAGUGGCACCGAGUCGGUGC | |
| | UUUU | |
| gRNA-#61 | UAUAAAAGCCCCAGGCUGGGGUUUUAGAGCUAGAAAUAGCAAGUUAAA | 614 |
| | AUAAGGCUAGUCCGUUAUCAACUUGAAAAAGUGGCACCGAGUCGGUGC | |
| | UUUU | |
| gRNA-#62 | UCACUUCUCCUGAGCUAGGCGUUUUAGAGCUAGAAAUAGCAAGUUAAA | 615 |
| | AUAAGGCUAGUCCGUUAUCAACUUGAAAAAGUGGCACCGAGUCGGUGC | |
| | UUUU | |
| gRNA-#63 | UCAGAUUGGCAGGGAUAAGCGUUUUAGAGCUAGAAAUAGCAAGUUAAA | 616 |
| | AUAAGGCUAGUCCGUUAUCAACUUGAAAAAGUGGCACCGAGUCGGUGC | |
| | UUUU | |
| gRNA-#64 | UCAGGAGAAGUGAGUAUAAAGUUUUAGAGCUAGAAAUAGCAAGUUAAA | 617 |
| | AUAAGGCUAGUCCGUUAUCAACUUGAAAAAGUGGCACCGAGUCGGUGC | |
| | UUUU | |
| gRNA-#65 | UCUGACUGCAAACCUGCUGAUGUUUUAGUACUCUGUAAUGAAAAUUAC | 618 |
| | AGAAUCUACUAAAACAAGGCAAAAUGCCGUGUUUAUCUCGUCAACUUG | |
| | UUGGCGAGAUUUU | |
| gRNA-#66 | UGAGCUAGGCUGCUUAUCCCGUUUUAGAGCUAGAAAUAGCAAGUUAAA | 619 |
| | AUAAGGCUAGUCCGUUAUCAACUUGAAAAAGUGGCACCGAGUCGGUGC | |
| | UUUU | |
| gRNA-#67 | UGCCAAUCUGACUGCAAACCGUUUUAGAGCUAGAAAUAGCAAGUUAAA | 620 |
| | AUAAGGCUAGUCCGUUAUCAACUUGAAAAAGUGGCACCGAGUCGGUGC | |
| | UUUU | |
| gRNA-#68 | UGCUCUAAUCUCUCUAGACAGUUUUAGAGCUAGAAAUAGCAAGUUAAA | 621 |
| | AUAAGGCUAGUCCGUUAUCAACUUGAAAAAGUGGCACCGAGUCGGUGC | |
| | UUUU | |
| gRNA-#69 | UGUGAUGGCUGCUCCCAGCCGUUUUAGAGCUAGAAAUAGCAAGUUAAA | 622 |
| | AUAAGGCUAGUCCGUUAUCAACUUGAAAAAGUGGCACCGAGUCGGUGC | |
| | UUUU | |
| gRNA-#70 | UUGGCAGGGAUAAGCAGCCUGUUUUAGAGCUAGAAAUAGCAAGUUAAA | 623 |
| | AUAAGGCUAGUCCGUUAUCAACUUGAAAAAGUGGCACCGAGUCGGUGC | |
| | UUUU | |
| gRNA-#71 | UUUUAUACUCACUUCUCCUGGUUUUAGAGCUAGAAAUAGCAAGUUAAA | 624 |
| | AUAAGGCUAGUCCGUUAUCAACUUGAAAAAGUGGCACCGAGUCGGUGC | |
| | UUUU | |
| gRNA_361 | UAUAGGAAAACCAGUGAGUC | 475 |
| gRNA_362 | UACUCACCUCUGCAUGCUCA | 476 |
| gRNA_363 | ACUCACCUCUGCAUGCUCAU | 625 |
| gRNA_364 | UACCACCUAUGAGAGAAGAC | 626 |
| gRNA_365 | AUACUCACCUCUGCAUGCUCA | 627 |
| gRNA_366 | ACUGGUUUUCCUAUAAGGUGU | 628 |
| gRNA_367 | UUGGCAGGAUGGCUUCUCAUCG | 629 |
| gRNA_368 | UCCUAUAAGGUGUGAAAGUCUG | 630 |
| gRNA_369 | UGAGCCCAUGCAGCUCUCCAGA | 631 |
| gRNA_370 | CUCCUCAGUUGUGAGCCCAUGC | 632 |
| gRNA_371 | GUAGAAGGGAUAUACAAAGUGG | 633 |
| gRNA_372 | CCACUUUGUAUAUCCCUUCUAC | 634 |
| gRNA_373 | GGUGUCUAUUUCCACUUUGUAU | 635 |
| gRNA_374 | CAUGAGCAUGCAGAGGUGAGUA | 636 |
| gRNA_375 | GGCUAUCGUCACCAAUCCCA | 637 |
| gRNA_376 | GCUAUCGUCACCAAUCCCAA | 638 |
| gRNA_377 | GGCUAUCGUCACCAAUCCCA | 637 |
| gRNA_361 | UAUAGGAAAACCAGUGAGUC | 475 |
| gRNA_362 | UACUCACCUCUGCAUGCUCA | 476 |
| gRNA_363 | ACUCACCUCUGCAUGCUCAU | 625 |
| gRNA_364 | UACCACCUAUGAGAGAAGAC | 626 |
| gRNA_365 | AUACUCACCUCUGCAUGCUCA | 627 |
| gRNA_366 | ACUGGUUUUCCUAUAAGGUGU | 628 |
| gRNA_367 | UUGGCAGGAUGGCUUCUCAUCG | 629 |
| gRNA_368 | UCCUAUAAGGUGUGAAAGUCUG | 630 |
| gRNA_369 | UGAGCCCAUGCAGCUCUCCAGA | 631 |
| gRNA_370 | CUCCUCAGUUGUGAGCCCAUGC | 632 |
| gRNA_371 | GUAGAAGGGAUAUACAAAGUGG | 633 |
| gRNA_372 | CCACUUUGUAUAUCCCUUCUAC | 634 |
| gRNA_373 | GGUGUCUAUUUCCACUUUGUAU | 635 |
| gRNA_374 | CAUGAGCAUGCAGAGGUGAGUA | 636 |
| gRNA_375 | GGCUAUCGUCACCAAUCCCA | 637 |
| gRNA_376 | GCUAUCGUCACCAAUCCCAA | 638 |
| gRNA_377 | GGCUAUCGUCACCAAUCCCA | 637 |
| gRNA1747 | AAGAGAGAAUAAGUAACCCAU | 472 |
| gRNA1748 | AAGCAGCCUAGCUCAGGAGAA | 654 |
| gRNA1749 | AAGUCCACUCAUUCUUGGCA | 655 |
| gRNA1750 | ACGAUGAGAAGCCAUCCUGCC | 656 |
| gRNA1751 | AGACAAGGUUCAUAUUUGUA | 657 |
| gRNA1752 | AGGCUGGGAGCAGCCAUCAC | 658 |
| gRNA1753 | AUAAGUAACCCAUACAAAUA | 659 |
| gRNA1754 | AUACUCACUUCUCCUGAGCU | 660 |
| gRNA1755 | AUUAUUGACUUAGUCAACAA | 661 |
| gRNA1756 | CAAAUAUGAACCUUGUCUAG | 662 |
| gRNA1757 | CAGAAGUCCACUCAUUCUUGG | 663 |
| gRNA1758 | CAGGCUGGGAGCAGCCAUCAC | 664 |
| gRNA1759 | CCAUCCUGCCAAGAAUGAGU | 665 |
| gRNA1760 | CCUGCUGAUUCUGAUUAUUGA | 666 |
| gRNA1761 | CGAUGCUCUAAUCUCUCUAGA | 667 |
| gRNA1762 | CUAAGUCAAUAAUCAGAAUCA | 668 |
| gRNA1763 | CUAGACAAGGUUCAUAUUUGU | 669 |
| gRNA1764 | GAACCUUGUCUAGAGAGAUU | 670 |
| gRNA1765 | GAAGUCCACUCAUUCUUGGC | 671 |
| gRNA1766 | GAAUCAGCAGGUUUGCAGUC | 672 |
| gRNA1767 | GAAUGAGUGGACUUCUGUGA | 673 |
| gRNA1768 | GACUGCAAACCUGCUGAUUC | 674 |
| gRNA1769 | GACUUAGUCAACAAAGAGAGA | 675 |
| gRNA1770 | GAUAAGCAGCCUAGCUCAGG | 676 |
| gRNA1771 | GAUGAGAAGCCAUCCUGCCA | 677 |
| gRNA1773 | GCUUUUAUACUCACUUCUCC | 654 |
| gRNA1774 | GGAUAAGCAGCCUAGCUCAGG | 655 |
| gRNA1775 | GUCUAGAGAGAUUAGAGCAU | 656 |
| gRNA1776 | GUGAUGGCUGCUCCCAGCCU | 657 |
| gRNA1777 | UACUUAUUCUCUCUUUGUUGA | 658 |
| gRNA1778 | UAUUCUCUCUUUGUUGACUAA | 659 |
| gRNA1779 | UAUUGACUUAGUCAACAAAG | 660 |
| gRNA1780 | UAUUGACUUAGUCAACAAAGA | 661 |
| gRNA1781 | UCAGAAUCAGCAGGUUUGCAG | 662 |
| gRNA1782 | UCCACUCAUUCUUGGCAGGA | 663 |
| gRNA1783 | UCUCUCUUUGUUGACUAAGUC | 664 |
| gRNA1784 | UGAGAAGCCAUCCUGCCAAGA | 665 |
| gRNA1785 | UGAGCUAGGCUGCUUAUCCCU | 666 |
| gRNA1786 | UGAGUAUAAAAGCCCCAGGC | 667 |
| gRNA1787 | UGAUGGCUGCUCCCAGCCUG | 668 |
| gRNA1788 | UGCCAAGAAUGAGUGGACUUC | 669 |
| gRNA1789 | UGCCAAUCUGACUGCAAACCU | 670 |
| gRNA1790 | UGUUGACUAAGUCAAUAAUC | 671 |
| gRNA1791 | UUGACUUAGUCAACAAAGAG | 672 |
| gRNA1792 | UUUGUUGACUAAGUCAAUAAU | 673 |
| gRNA1746 | AACCUGCUGAUUCUGAUUAU | 674 |
| gRNA1594 | CAACUUACCCAGAGGCAAAU | 675 |
| gRNA1595 | AAUGGCUCCCAGGUGUCAUC | 676 |
| gRNA1596 | GGCUCCCAGGUGUCAUCAGC | 677 |
| gRNA1597 | CUCUCAUAGGUGGUAUUCAC | 653 |
| gRNA1598 | UAUAGGAAAACCAGUGAGUC | 475 |
| gRNA1599 | UACUCACCUCUGCAUGCUCA | 476 |
| gRNA1600 | GCAACUUACCCAGAGGCAAA | 474 |
| gRNA1601 | UCUGUAUACUCACCUCUGCA | 707 |
| gRNA1602 | GAAACACUCACCGUAGGGCC | 708 |
| gRNA1603 | CUCUACACCCAGGGCACCGG | 709 |
| gRNA1604 | ACACCUUAUAGGAAAACCAG | 710 |
| gRNA1605 | AUAGGAAAACCAGUGAGUCU | 711 |
| gRNA1606 | ACUCACCUCUGCAUGCUCAU | 625 |
| gRNA1607 | CUCACCGUAGGGCCAGCCUC | 713 |
| gRNA-#1 | AAAAGCCCCAGGCUGGGAGC | 714 |
| gRNA-#2 | AAGUGAGUAUAAAAGCCCCA | 715 |
| gRNA-#3 | AAUAAUCAGAAUCAGCAGGUU | 716 |
| gRNA-#4 | AAUAUGAACCUUGUCUAGAG | 717 |
| gRNA-#5 | AAUGAGUGGACUUCUGUGAU | 718 |
| gRNA-#6 | ACAAAUAUGAACCUUGUCUAG | 719 |
| gRNA-#7 | ACAGAAGUCCACUCAUUCUU | 720 |
| gRNA-#8 | ACCUUGUCUAGAGAGAUUAG | 721 |
| gRNA-#9 | AGAAGCCAUCCUGCCAAGAA | 722 |
| gRNA-#10 | AGCAGGUUUGCAGUCAGAUU | 723 |
| gRNA-#11 | AGGGAUAAGCAGCCUAGCUC | 724 |
| gRNA-#12 | AGGUUUGCAGUCAGAUUGGC | 725 |
| gRNA-#13 | AGUAUAAAAGCCCCAGGCUG | 726 |
| gRNA-#14 | AGUCAAUAAUCAGAAUCAGC | 727 |
| gRNA-#15 | AUAAUCAGAAUCAGCAGGUU | 728 |
| gRNA-#16 | UUGACUUAGUCAACAAAGAGAG | 729 |
| gRNA-#17 | UCUCUUUGUUGACUAAGUCAAU | 730 |
| gRNA-#18 | UGAUUAUUGACUUAGUCAACAA | 731 |
| gRNA-#19 | UUGGCAGGAUGGCUUCUCAUCG | 629 |
| (gRNA_367) | | |
| gRNA-#20 | ACUUAGUCAACAAAGAGAGAAU | 733 |
| gRNA-#21 | GCAGGGAUAAGCAGCCUAGCUC | 734 |
| gRNA-#22 | GUAUGGGUUACUUAUUCUCUCU | 735 |
| gRNA-#23 | CAAGAAUGAGUGGACUUCUG | 736 |
| gRNA-#24 | CAAUCUGACUGCAAACCUGC | 737 |
| gRNA-#25 | CACAGAAGUCCACUCAUUCU | 738 |
| gRNA-#26 | CAGACGAUGAGAAGCCAUCC | 739 |
| gRNA-#27 | CAGCAGGUUUGCAGUCAGAU | 740 |
| gRNA-#28 | CAGGAUGGCUUCUCAUCGUC | 741 |
| gRNA-#29 | CAGGUUUGCAGUCAGAUUGGC | 742 |
| gRNA-#30 | CAGUCAGAUUGGCAGGGAUA | 743 |
| gRNA-#31 | CCACUCAUUCUUGGCAGGAU | 744 |
| gRNA-#32 | CUAAGUCAAUAAUCAGAAUC | 745 |
| gRNA-#33 | GUCAACAAAGAGAGAAUAAGUA | 746 |
| gRNA-#34 | CUUAUCCCUGCCAAUCUGAC | 747 |
| gRNA-#35 | UCCCUGCCAAUCUGACUGCAAA | 748 |
| gRNA-#36 | UUCUCUCUUUGUUGACUAAGUC | 749 |
| gRNA-#37 | UCCUGAGCUAGGCUGCUUAUCC | 750 |
| gRNA-#38 | CUUCUGUGAUGGCUGCUCCC | 751 |
| gRNA-#39 | UGUGAUGGCUGCUCCCAGCCUG | 752 |
| gRNA-#40 | GCAGGAUGGCUUCUCAUCGUCU | 753 |
| gRNA-#41 | UCUAGAGAGAUUAGAGCAUCGG | 754 |
| gRNA-#42 | GUUGACUAAGUCAAUAAUCAGA | 755 |
| gRNA-#43 | UAUACUCACUUCUCCUGAGCUA | 756 |
| gRNA-#44 | GAAGUGAGUAUAAAAGCCCC | 757 |
| gRNA-#45 | GACAAGGUUCAUAUUUGUAU | 758 |
| gRNA-#46 | GAGUAUAAAAGCCCCAGGCU | 759 |
| gRNA-#47 | GAGUGGACUUCUGUGAUGGC | 760 |
| gRNA-#48 | GAUGGCUGCUCCCAGCCUGG | 761 |
| gRNA-#49 | GCAGCCUAGCUCAGGAGAAG | 762 |
| gRNA-#50 | GCUGCUUAUCCCUGCCAAUC | 763 |
| gRNA-#51 | GGGAUAAGCAGCCUAGCUCA | 764 |
| gRNA-#52 | GGUUUGCAGUCAGAUUGGCA | 765 |
| gRNA-#53 | GUUACUUAUUCUCUCUUUGU | 766 |
| gRNA-#54 | CUUAUUCUCUCUUUGUUGACUA | 767 |
| gRNA-#55 | AUAUUUGUAUGGGUUACUUAUU | 768 |
| gRNA-#56 | ACUAAGUCAAUAAUCAGAAUCA | 769 |
| gRNA-#57 | GUUUGCAGUCAGAUUGGCAG | 770 |
| gRNA-#58 | GCAGUCAGAUUGGCAGGGAUAA | 771 |
| gRNA-#59 | UACAAAUAUGAACCUUGUCU | 772 |
| gRNA-#60 | UACUCACUUCUCCUGAGCUA | 773 |
| gRNA-#61 | UAUAAAAGCCCCAGGCUGGG | 774 |
| gRNA-#62 | UCACUUCUCCUGAGCUAGGC | 775 |
| gRNA-#63 | UCAGAUUGGCAGGGAUAAGC | 776 |
| gRNA-#64 | UCAGGAGAAGUGAGUAUAAA | 777 |
| gRNA-#65 | UCUGACUGCAAACCUGCUGAU | 778 |
| gRNA-#66 | UGAGCUAGGCUGCUUAUCCC | 779 |
| gRNA-#67 | UGCCAAUCUGACUGCAAACC | 780 |
| gRNA-#68 | UGCUCUAAUCUCUCUAGACA | 781 |
| gRNA-#69 | UGUGAUGGCUGCUCCCAGCC | 782 |
| gRNA-#70 | UUGGCAGGGAUAAGCAGCCU | 783 |
| gRNA-#71 | UUUUAUACUCACUUCUCCUG | 784 |
| gRNA-#54 | CUUAUUCUCUCUUUGUUGACUA | 767 |
| gRNA-#55 | AUAUUUGUAUGGGUUACUUAUU | 768 |
| gRNA-#56 | ACUAAGUCAAUAAUCAGAAUCA | 769 |
| gRNA-#57 | GUUUGCAGUCAGAUUGGCAG | 770 |
| gRNA-#58 | GCAGUCAGAUUGGCAGGGAUAA | 771 |
| gRNA-#59 | UACAAAUAUGAACCUUGUCU | 772 |
| gRNA-#60 | UACUCACUUCUCCUGAGCUA | 773 |
| gRNA-#61 | UAUAAAAGCCCCAGGCUGGG | 774 |
| gRNA-#62 | UCACUUCUCCUGAGCUAGGC | 775 |
| gRNA-#63 | UCAGAUUGGCAGGGAUAAGC | 776 |
| gRNA-#64 | UCAGGAGAAGUGAGUAUAAA | 777 |
| gRNA-#65 | UCUGACUGCAAACCUGCUGAU | 778 |
| gRNA-#66 | UGAGCUAGGCUGCUUAUCCC | 779 |
| gRNA-#67 | UGCCAAUCUGACUGCAAACC | 780 |
| gRNA-#68 | UGCUCUAAUCUCUCUAGACA | 781 |
| gRNA-#69 | UGUGAUGGCUGCUCCCAGCC | 782 |
| gRNA-#70 | UUGGCAGGGAUAAGCAGCCU | 783 |
| gRNA-#71 | UUUUAUACUCACUUCUCCUG | 784 |
| gRNA-#54 | CUUAUUCUCUCUUUGUUGACUA | 767 |
| gRNA-#55 | AUAUUUGUAUGGGUUACUUAUU | 768 |
| gRNA-#56 | ACUAAGUCAAUAAUCAGAAUCA | 769 |
| gRNA-#57 | GUUUGCAGUCAGAUUGGCAG | 770 |
| gRNA-#58 | GCAGUCAGAUUGGCAGGGAUAA | 771 |
| gRNA-#59 | UACAAAUAUGAACCUUGUCU | 772 |
| gRNA-#60 | UACUCACUUCUCCUGAGCUA | 773 |
| gRNA-#61 | UAUAAAAGCCCCAGGCUGGG | 774 |
| gRNA-#62 | UCACUUCUCCUGAGCUAGGC | 775 |
| gRNA-#63 | UCAGAUUGGCAGGGAUAAGC | 776 |
| gRNA-#64 | UCAGGAGAAGUGAGUAUAAA | 777 |
| gRNA-#65 | UCUGACUGCAAACCUGCUGAU | 778 |
| gRNA-#66 | UGAGCUAGGCUGCUUAUCCC | 779 |
| gRNA-#67 | UGCCAAUCUGACUGCAAACC | 780 |
| gRNA-#68 | UGCUCUAAUCUCUCUAGACA | 781 |
| gRNA-#69 | UGUGAUGGCUGCUCCCAGCC | 782 |
| gRNA-#70 | UUGGCAGGGAUAAGCAGCCU | 783 |
| gRNA-#71 | UUUUAUACUCACUUCUCCUG | 784 |
| | GCCAUCCUGCCAAGAACGAG | 473 |
| | GCAACUUACCCAGAGGCAAA | 474 |
| | UAUAGGAAAACCAGUGAGUC | 475 |
| | UACUCACCUCUGCAUGCUCA | 476 |
| | GCCAUCCUGCCAAGAACGAG | 473 |
| | GCCAUCCUGCCAAGAACGAG | 473 |
| | GCAACUUACCCAGAGGCAAA | 474 |
| | UAUAGGAAAACCAGUGAGUC | 475 |
| | UACUCACCUCUGCAUGCUCA | 476 |
| | GCCAUCCUGCCAAGAACGAG | 473 |
| | AUCCUGCCAAGAACGAGGUUUUAGAGCUAGAAAUAGCAAGUUAAAAUA | 1214 |
| | AGGCUAGUCCGUUAUCAACUUGAAAAAGUGGCACCGAGUCGGUGCUUU | |
| | U | |
| | GCAACUUACCCAGAGGCAAAGUUUUAGAGCUAGAAAUAGCAAGUUAAA | 499 |
| | AUAAGGCUAGUCCGUUAUCAACUUGAAAAAGUGGCACCGAGUCGGUGC | |
| | UUUU | |
| | UAUAGGAAAACCAGUGAGUCGUUUUAGAGCUAGAAAUAGCAAGUUAAA | 497 |
| | AUAAGGCUAGUCCGUUAUCAACUUGAAAAAGUGGCACCGAGUCGGUGC | |
| | UUUU | |
| | UACUCACCUCUGCAUGCUCAGUUUUAGAGCUAGAAAUAGCAAGUUAAA | 480 |
| | AUAAGGCUAGUCCGUUAUCAACUUGAAAAAGUGGCACCGAGUCGGUGC | |
| | UUUU | |
| | GCCAUCCUGCCAAGAACGAGGUUUUAGAGCUAGAAAUAGCAAGUUAAA | 1044 |
| | AUAAGGCUAGUCCGUUAUCAACUUGAAAAAGUGGCACCGAGUCGGUGC | |
| | UUUU | |
| | UAUAGGAAAACCAGUGAGUCGUUUUAGAGCUAGAAAUAGCAAGUUAAA | 497 |
| | AUAAGGCUAGUCCGUUAUCAACUUGAAAAAGUGGCACCGAGUCGGUGC | |
| | UUUU | |
| | GCCAUCCUGCCAAGAACGAGGUUUUAGAGCUAGAAAUAGCAAGUUAAA | 1044 |
| | AUAAGGCUAGUCCGUUAUCAACUUGAAAAAGUGGCACCGAGUCGGUGC | |
| | UUUU | |
| | GCAACUUACCCAGAGGCAAAGUUUUAGAGCUAGAAAUAGCAAGUUAAA | 499 |
| | AUAAGGCUAGUCCGUUAUCAACUUGAAAAAGUGGCACCGAGUCGGUGC | |
| | UUUU | |
| | UAUAGGAAAACCAGUGAGUCGUUUUAGAGCUAGAAAUAGCAAGUUAAA | 497 |
| | AUAAGGCUAGUCCGUUAUCAACUUGAAAAAGUGGCACCGAGUCGGUGC | |
| | UUUU | |
| | UACUCACCUCUGCAUGCUCAGUUUUAGAGCUAGAAAUAGCAAGUUAAA | 480 |
| | AUAAGGCUAGUCCGUUAUCAACUUGAAAAAGUGGCACCGAGUCGGUGC | |
| | UUUU | |
| | GCCAUCCUGCCAAGAACGAGGUUUUAGAGCUAGAAAUAGCAAGUUAAA | 1044 |
| | AUAAGGCUAGUCCGUUAUCAACUUGAAAAAGUGGCACCGAGUCGGUGC | |
| | UUUU | |
| | GCCAUCCUGCCAAGAACGAGGUUUUAGAGCUAGAAAUAGCAAGUUAAA | 1044 |
| | AUAAGGCUAGUCCGUUAUCAACUUGAAAAAGUGGCACCGAGUCGGUGC | |
| | UUUU | |
| | GCAACUUACCCAGAGGCAAAGUUUUAGAGCUAGAAAUAGCAAGUUAAA | 499 |
| | AUAAGGCUAGUCCGUUAUCAACUUGAAAAAGUGGCACCGAGUCGGUGC | |
| | UUUU | |
| | UAUAGGAAAACCAGUGAGUCGUUUUAGAGCUAGAAAUAGCAAGUUAAA | 497 |
| | AUAAGGCUAGUCCGUUAUCAACUUGAAAAAGUGGCACCGAGUCGGUGC | |
| | UUUU | |
| | UACUCACCUCUGCAUGCUCAGUUUUAGAGCUAGAAAUAGCAAGUUAAA | 480 |
| | AUAAGGCUAGUCCGUUAUCAACUUGAAAAAGUGGCACCGAGUCGGUGC | |
| | UUUU | |
| | GCCAUCCUGCCAAGAACGAGGUUUUAGAGCUAGAAAUAGCAAGUUAAA | 1044 |
| | AUAAGGCUAGUCCGUUAUCAACUUGAAAAAGUGGCACCGAGUCGGUGC | |
| | UUUU | |
| GA519 | GCCAUCCUGCCAAGAACGAGGUUUUAGAGCUAGAAAUAGCAAGUUAAA | 1044 |
| | AUAAGGCUAGUCCGUUAUCAACUUGAAAAAGUGGCACCGAGUCGGUGC | |
| | UUUU | |
| GA458 | GCCAUCCUGCCAAGAACGAGGUUUUAGAGCUAGAAAUAGCAAGUUAAA | 1044 |
| | AUAAGGCUAGUCCGUUAUCAACUUGAAAAAGUGGCACCGAGUCGGUGC | |
| | UUUU | |
| GA459 | GCAACUUACCCAGAGGCAAAGUUUUAGAGCUAGAAAUAGCAAGUUAAA | 499 |
| | AUAAGGCUAGUCCGUUAUCAACUUGAAAAAGUGGCACCGAGUCGGUGC | |
| | UUUU | |
| GA460 | UAUAGGAAAACCAGUGAGUCGUUUUAGAGCUAGAAAUAGCAAGUUAAA | 497 |
| | AUAAGGCUAGUCCGUUAUCAACUUGAAAAAGUGGCACCGAGUCGGUGC | |
| | UUUU | |
| GA520 | UAUAGGAAAACCAGUGAGUCGUUUUAGAGCUAGAAAUAGCAAGUUAAA | 497 |
| | AUAAGGCUAGUCCGUUAUCAACUUGAAAAAGUGGCACCGAGUCGGUGC | |
| | UUUU | |
| GA461 | UACUCACCUCUGCAUGCUCAGUUUUAGAGCUAGAAAUAGCAAGUUAAA | 480 |
| | AUAAGGCUAGUCCGUUAUCAACUUGAAAAAGUGGCACCGAGUCGGUGC | |
| | UUUU | |
| GA457 | GCCAUCCUGCCAAGAACGAGGUUUUAGAGCUAGAAAUAGCAAGUUAAA | 1044 |
| | AUAAGGCUAGUCCGUUAUCAACUUGAAAAAGUGGCACCGAGUCGGUGC | |
| | UUUU | |
| GA519 | GCCAUCCUGCCAAGAACGAGGUUUUAGAGCUAGAAAUAGCAAGUUAAA | 1044 |
| | AUAAGGCUAGUCCGUUAUCAACUUGAAAAAGUGGCACCGAGUCGGUGC | |
| | UUUU | |
| GA458 | GCAACUUACCCAGAGGCAAAGUUUUAGAGCUAGAAAUAGCAAGUUAAA | 499 |
| | AUAAGGCUAGUCCGUUAUCAACUUGAAAAAGUGGCACCGAGUCGGUGC | |
| | UUUU | |
| GA459 | UAUAGGAAAACCAGUGAGUCGUUUUAGAGCUAGAAAUAGCAAGUUAAA | 497 |
| | AUAAGGCUAGUCCGUUAUCAACUUGAAAAAGUGGCACCGAGUCGGUGC | |
| | UUUU | |
| GA460 | UAUAGGAAAACCAGUGAGUCGUUUUAGAGCUAGAAAUAGCAAGUUAAA | 497 |
| | AUAAGGCUAGUCCGUUAUCAACUUGAAAAAGUGGCACCGAGUCGGUGC | |
| | UUUU | |
| GA520 | UACUCACCUCUGCAUGCUCAGUUUUAGAGCUAGAAAUAGCAAGUUAAA | 480 |
| | AUAAGGCUAGUCCGUUAUCAACUUGAAAAAGUGGCACCGAGUCGGUGC | |
| | UUUU | |
| GA461 | GCCATCCTGCCAAGAATGAGGUUUUAGAGCUAGAAAUAGCAAGUUAAA | 1045 |
| | AUAAGGCUAGUCCGUUAUCAACUUGAAAAAGUGGCACCGAGUCGGUGC | |
| | UUUU | |
| GA457 | GCCAUCCUGCCAAGAACGAGGUUUUAGAGCUAGAAAUAGCAAGUUAAA | 1215 |
| | AUAAGGCUAGUCCGUUAUCAACUUGAAAAAGUGGCACCGAGUCGGUGC | |
| | UU | |
| GA460 | UAUAGGAAAACCAGUGAGUCGUUUUAGAGCUAGAAAUAGCAAGUUAAA | 497 |
| | AUAAGGCUAGUCCGUUAUCAACUUGAAAAAGUGGCACCGAGUCGGUGC | |
| | UUUU | |
| gRNA_375 | GGCUAUCGUCACCAAUCCCA | 637 |
| gRNA_376 | GCUAUCGUCACCAAUCCCAA | 638 |
| gRNA_377 | GGCUAUCGUCACCAAUCCCA | 637 |

, a sequence provided in the sequence listing submitted herewith, wherein the guide polynucleotide does not comprise the sequence GCCAUCCUGCCAAGAAUGAG (SEQ ID NO: 467), wherein the lipid nanoparticle comprises an amino lipid according to any one of the following Formulas:
A) an amino lipid of Formula (Ia): 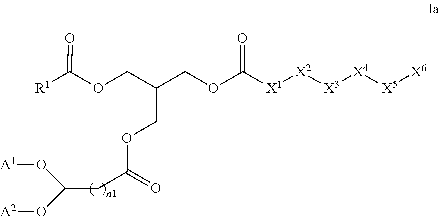
wherein:
R¹ is C₉-C₂₀ alkyl or C₉-C₂₀ alkenyl with 1-3 units of unsaturation;
X¹ and X² are each independently absent or selected from —O—, —NR²— and 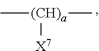
wherein each R² is independently hydrogen or C₁-C₆ alkyl;
each a is independently an integer between 1 and 6;
X³ and X⁴ are each independently absent or selected from the group consisting of: 4- to 8-membered heterocyclyl optionally substituted with 1 or 2 C₁-C₆ alkyl groups, 5- to 6-membered heteroaryl optionally substituted with 1 or 2 C₁-C₆ alkyl groups, 5- to 6-membered aryl optionally substituted with 1 or 2 C₁-C₆ alkyl groups, 4- to 7-membered cycloalkyl optionally substituted with 1 or 2 C₁-C₆ alkyl groups, —O— and —NR³—, wherein each R³ is a independently a hydrogen atom or C₁-C₆ alkyl and wherein X¹-X²-X³-X⁴ does not contain any oxygen-oxygen, oxygen-nitrogen or nitrogen-nitrogen bonds;
X⁵ is —(CH₂)_{b}—, wherein b is an integer between 0 and 6;
X⁶ is hydrogen, C₁-C₆ alkyl, 5- to 6-membered heteroaryl optionally substituted with 1 or 2 C₁-C₆ alkyl groups, or —NR⁴R⁵, wherein R⁴ and R⁵ are each independently hydrogen or C₁-C₆ alkyl; or alternatively R⁴ and R⁵ join together with the nitrogen to which they are bound to form a 4- to 7-membered heterocyclyl optionally substituted with 1 or 2 C₁-C₆ alkyl groups, wherein the heterocyclyl optionally includes an additional heteroatom selected from oxygen, sulfur, and nitrogen;
each X⁷ is independently hydrogen, hydroxyl or —NR⁶R⁷, wherein R⁶ and R⁷ are each independently hydrogen or C₁-C₆ alkyl; or alternatively R⁶ and R⁷ join together with the nitrogen to which they are bound to form a 4- to 7-membered heterocyclyl optionally substituted with 1 or 2 C₁-C₆ alkyl groups, wherein the heterocyclyl optionally includes an additional heteroatom selected from oxygen, sulfur, and nitrogen;
at least one of X¹, X², X³, X⁴, and X⁵ is present;
A¹ and A² are each independently selected from the group consisting of: C₅-C₁₂ haloalkyl, C₅-C₁₂ alkenyl, C₅-C₁₂ alkynyl, (C₅-C₁₂ alkoxy)-(CH₂)ₙ₂—, (C₅-C₁₀ aryl)-(CH₂)ₙ₃-optionally ring substituted with one or two halo, C₁-C₆ alkyl, C₁-C₆ haloalkyl, or C₁-C₆ alkoxy groups, and (C₃-C₈ cycloalkyl)-(CH₂)ₙ₄-optionally ring substituted with 1 or 2 C₁-C₆ alkyl groups; or alternatively A¹ and A² join together with the atoms to which they are bound to form a 5- to 6-membered cyclic acetal substituted with 1 or 2 C₄-C₁₀ alkyl groups;
n1, n2 and n3 are each individually an integer between 1 and 4; and
n4 is an integer between zero and 4;
B) an amino lipid of Formula (Ib): 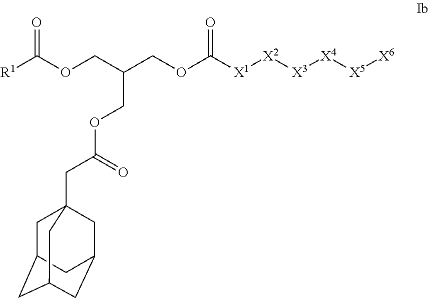
wherein:
R¹ is C₉-C₂₀ alkyl or C₉-C₂₀ alkenyl with 1-3 units of unsaturation;
X¹ and X² are each independently absent or selected from —O—, NR², and 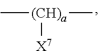
wherein R² is C₁-C₆ alkyl, and wherein X¹ and X² are not both —O— or NR²;
a is an integer between 1 and 6;
X³ and X⁴ are each independently absent or selected from the group consisting of: 4- to 7-membered heterocyclyl optionally substituted with 1 or 2 C₁-C₆ alkyl groups, 5- to 6-membered heteroaryl optionally substituted with 1 or 2 C₁-C₆ alkyl groups, and —NR³—, wherein each R³ is a hydrogen atom or C₁-C₆ alkyl;
X⁵ is —(CH₂)_{b}—, wherein b is an integer between 0 and 6;
X⁶ is hydrogen, C₁-C₆ alkyl, 5- to 6-membered heteroaryl optionally substituted with 1 or 2 C₁-C₆ alkyl groups, or —NR⁴R⁵, wherein R⁴ and R⁵ are each independently hydrogen or C₁-C₆ alkyl; or alternatively R⁴ and R⁵ join together with the nitrogen to which they are bound to form a 4- to 7-membered heterocyclyl optionally substituted with 1 or 2 C₁-C₆ alkyl groups, wherein the heterocyclyl optionally includes an additional heteroatom selected from oxygen, sulfur, and nitrogen;
X⁷ is hydrogen or —NR⁶R⁷, wherein R⁶ and R⁷ are each independently hydrogen or C₁-C₆ alkyl; or alternatively R⁶ and R⁷ join together with the nitrogen to which they are bound to form a 4- to 7-membered heterocyclyl optionally substituted with 1 or 2 C₁-C₆ alkyl groups, wherein the heterocyclyl optionally includes an additional heteroatom selected from oxygen, sulfur, and nitrogen;
at least one of X¹, X², X³, X⁴, and X⁵ is present; and
provided that when either X¹ or X² is —O—, neither X³ nor X⁴ is 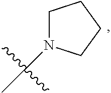 [SMILES: *N1CCCC1]
and when either X¹ or X² is —O—, R⁴ and R⁵ are not both ethyl;
C) an amino lipid of Formula (Ic): 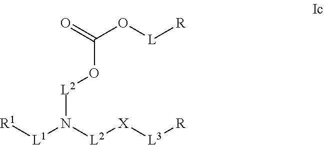
or its N-oxide, or a salt thereof, wherein
L1 is C₁-6 alkylenyl, or C₂-6 heteroalkylenyl;
each L² is independently C₂-10 alkylenyl, or C₃-10 heteroalkylenyl;
L is absent, C₁-10 alkylenyl, or C₂-10 heteroalkylenyl;
L³ is absent, C₁-10 alkylenyl, or C₂-10 heteroalkylenyl;
X is absent, —OC(O)—, —C(O)O—, or —OC(O)O—; 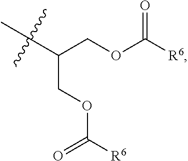
each R is independently hydrogen, or an optionally substituted group selected from C₆-20 aliphatic, C₆-20 haloaliphatic, a 3- to 7-membered cycloaliphatic ring, 1-adamantyl, 2-adamantyl, sterolyl, and phenyl;
R¹ is hydrogen, a 3- to 7-membered cycloaliphatic ring, a 3- to 7-membered heterocyclic ring comprising 1-3 heteroatoms independently selected from nitrogen, oxygen, and sulfur, —OR², —C(O)OR², —C(O) SR², —OC(O)R², —OC(O)OR², —CN, —N(R²)₂, —C(O)N(R²)₂, —NR²C(O)R², —OC(O)N(R²)₂, —N(R²)C(O)OR², —NR²S(O)₂R², —NR²C(O)N(R²)₂, —NR²C(S)N(R²)₂, —NR²C(NR²)N(R²)₂, —NR²C(CHR²)N(R²)₂, —N(OR²)C(O)R², —N(OR²) S(O)₂R², —N(OR²)C(O)OR², —N(OR²)C(O)N(R²)₂, —N(OR²)C(S)N(R²)₂, —N(OR²)C(NR²)N(R²)₂, —N(OR²)C(CHR²)N(R²)₂, —C(NR²)N(R²)₂, —C(NR²)R², —C(O)N(R²)OR², —C(R²)N(R²)₂C(O)OR², 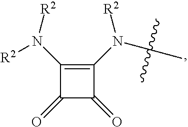
—CR² (OR²)R³, 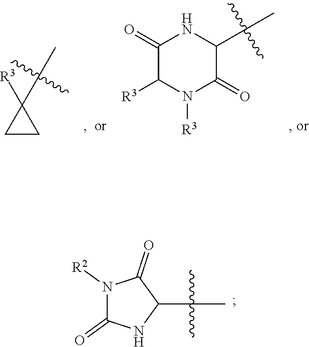
each R² is independently hydrogen, —CN, —NO₂, —OR⁴, —S(O)₂R⁴, —S(O)₂N(R⁴)₂, —(CH₂) n-R⁴, or an optionally substituted group selected from C₁-6 aliphatic, a 3- to 7-membered cycloaliphatic ring, and a 3- to 7-membered heterocyclic ring comprising 1-3 heteroatoms independently selected from nitrogen, oxygen, and sulfur, or
two occurrences of R², taken together with the atom(s) to which they are attached, form an optionally substituted 4- to 7-membered heterocyclic ring comprising 0-1 additional heteroatom selected from nitrogen, oxygen, and sulfur;
each R³ is independently-(CH₂) n-R⁴, or
two occurrences of R³, taken together with the atoms to which they are attached, form an optionally substituted 5- to 6-membered heterocyclic ring comprising 0-1 additional heteroatom selected from nitrogen, oxygen, and sulfur;
each R⁴ is independently
hydrogen, —OR⁵, —N(R⁵)₂, —OC(O)R⁵, —OC(O)OR⁵, —CN, —C(O)N(R⁵)₂,
—NR⁵C(O)R⁵, —OC(O)N(R⁵)₂, —N(R⁵)C(O)OR⁵, —NR⁵S(O)₂R⁵, —NR⁵° C. (O)N(R⁵)₂, —NR⁵° C. (S)N(R⁵)₂, —NR⁵C(NR⁵)N(R⁵)₂, or 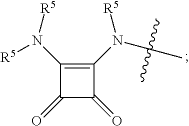
each R⁵ is independently hydrogen, optionally substituted C₁₋₆ aliphatic, or
two occurrences of R⁵, taken together with the atom(s) to which they are attached, form an optionally substituted 4- to 7-membered heterocyclic ring comprising 0-1 additional heteroatom selected from nitrogen, oxygen, and sulfur;
each R⁶ is independently C₄-12 aliphatic; and
n is 0 to 4;
D) an amino lipid of Formula (Id): 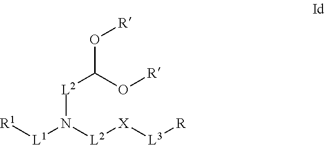
or its N-oxide, or a pharmaceutically acceptable salt thereof, wherein
L¹ is absent, C₁-6 alkylenyl, or C₂-6 heteroalkylenyl;
each L² is independently optionally substituted C₂₋₁₅ alkylenyl, or optionally substituted C₃₋₁₅ heteroalkylenyl;
L³ is absent, optionally substituted C₁₋₁₀ alkylenyl, or optionally substituted C₂₋₁₀ heteroalkylenyl;
X is absent, —OC(O)—, —C(O)O—, or —OC(O)O—;
each R′ is independently an optionally substituted group selected from C₄-12 aliphatic, 3- to 12-membered cycloaliphatic, 7- to 12-membered bridged bicyclic comprising 0-4 heteroatoms independently selected from nitrogen, oxygen, or sulfur, 1-adamantyl, 2-adamantyl, sterolyl, and phenyl;
R is hydrogen, 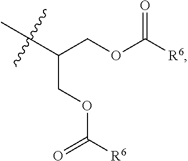
or an optionally substituted group selected from C₆-20 aliphatic, 3- to 12-membered cycloaliphatic, 7- to 12-membered bridged bicyclic comprising 0-4 heteroatoms independently selected from nitrogen, oxygen, or sulfur, 1-adamantyl, 2-adamantyl, sterolyl, and phenyl;
R¹ is hydrogen, optionally substituted phenyl, optionally substituted 3- to 7-membered cycloaliphatic, optionally substituted 3- to 7-membered heterocyclyl comprising 1-3 heteroatoms independently selected from nitrogen, oxygen, and sulfur, optionally substituted 5- to 6-membered monocyclic heteroaryl comprising 1-4 heteroatoms independently selected from nitrogen, oxygen, and sulfur, optionally substituted 8- to 10-membered bicyclic heteroaryl comprising 1-4 heteroatoms independently selected from nitrogen, oxygen, and sulfur, —OR², —C(O)OR², —C(O) SR², —OC(O)R², —OC(O)OR², —CN, —N(R²)₂, —C(O)N(R²)₂, —S(O)₂N(R²)₂, —NR²C(O)R², —OC(O)N(R²)₂, —N(R²)C(O)OR², —NR²S(O)₂R², —NR²C(O)N(R²)₂, —NR²C(S)N(R²)₂, —NR²C(NR²)N(R²)₂, —NR²C(CHR²)N(R²)₂, —N(OR²)C(O)R², —N(OR²) S(O)₂R², —N(OR²)C(O)OR², —N(OR²)C(O)N(R²)₂,
—N(OR²)C(S)N(R²)₂, —N(OR²)C(NR²)N(R²)₂, —N(OR²)C(CHR²)N(R²)₂, —C(NR²)N(R²)₂, —C(NR²)R², —C(O)N(R²)OR², —C(R²)N(R²)₂C(O)OR², —CR² (R³)₂, —OP(O) (OR²)₂, or —P(O)(OR²)₂; or
R¹ is 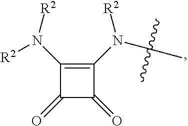
or a ring selected from 3- to 7-membered cycloaliphatic and 3- to 7-membered heterocyclyl comprising 1-3 heteroatoms independently selected from nitrogen, oxygen, and sulfur, wherein the cycloaliphatic or heterocyclyl ring is optionally substituted with 1-4 R² or R³ groups;
each R² is independently hydrogen, oxo, —CN, —NO₂, —OR⁴, —S(O)₂R⁴, —S(O)₂N(R⁴)₂, —(CH₂) n-R⁴, or an optionally substituted group selected from C₁-6 aliphatic, phenyl, 3- to 7-membered cycloaliphatic, 5- to 6-membered monocyclic heteroaryl comprising 1-4 heteroatoms independently selected from nitrogen, oxygen, and sulfur, and 3- to 7-membered heterocyclyl comprising 1-3 heteroatoms independently selected from nitrogen, oxygen, and sulfur; or
two occurrences of R², taken together with the atom(s) to which they are attached, form optionally substituted 4- to 7-membered heterocyclyl comprising 0-1 additional heteroatom selected from nitrogen, oxygen, and sulfur;
each R³ is independently —(CH₂)ₙ—R⁴; or
two occurrences of R³, taken together with the atom(s) to which they are attached, form optionally substituted 5- to 6-membered heterocyclyl comprising 0-1 additional heteroatom selected from nitrogen, oxygen, and sulfur;
each R⁴ is independently hydrogen, —OR⁵, —N(R⁵)₂, —OC(O)R⁵, —OC(O)OR⁵, —CN, —C(O)N(R⁵)₂,
—NR⁵C(O)R⁵, —OC(O)N(R⁵)₂, —N(R⁵)C(O)OR⁵, —NR⁵S(O)₂R⁵, —NR⁵° C. (O)N(R⁵)₂, —NR⁵C(S)N(R⁵)₂, —NR⁵° C. (NR⁵)N(R⁵)₂, or 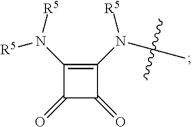
each R⁵ is independently hydrogen, or optionally substituted C₁₋₆ aliphatic; or
two occurrences of R⁵, taken together with the atom(s) to which they are attached, form optionally substituted 4- to 7-membered heterocyclyl comprising 0-1 additional heteroatom selected from nitrogen, oxygen, and sulfur;
each R⁶ is independently C₄-12 aliphatic; and
each n is independently 0 to 4;
E) an amino lipid of Formula (Ie): 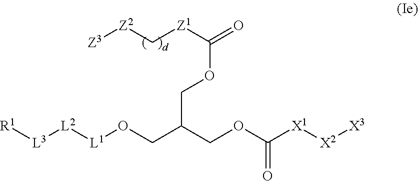
or a pharmaceutically acceptable salt thereof, wherein:
L¹ is a covalent bond, —C(O)—, or —OC(O)—;
L² is a covalent bond, an optionally substituted bivalent saturated or unsaturated, straight or branched C₁-C₁₂ hydrocarbon chain, or 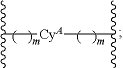
Cy^{A} is an optionally substituted ring selected from phenylene and 3- to 7-membered saturated or partially unsaturated carbocyclene;
each m is independently 0, 1, or 2;
L³ is a covalent bond, —C(O)—, —C(O)O—, —OC(O)—, —O—, or —OC(O)O—;
R¹ is 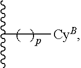
or an optionally substituted saturated or unsaturated, straight or branched C₁-C₂₀ hydrocarbon chain wherein 1-3 methylene units are optionally and independently replaced with —O— or —NR—;
Cy^{B} is an optionally substituted ring selected from 3- to 12-membered saturated or partially unsaturated carbocyclyl, 1-adamantyl, 2-adamantyl, 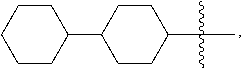 [SMILES: *C1CCC(C2CCCCC2)CC1]
sterolyl, and phenyl;
p is 0, 1, 2, or 3;
X¹ is a covalent bond, —O—, or —NR—;
X² is a covalent bond or an optionally substituted, bivalent saturated or unsaturated, straight or branched, C₁-C₁₂ hydrocarbon chain, wherein 1-3 methylene units are optionally and independently replaced with —O—, —NR—, or —Cy^{C}—;
Cy^{C} is an optionally substituted ring selected from 3- to 7-membered saturated or partially unsaturated carbocyclene, phenylene, 3- to 7-membered saturated or partially unsaturated heterocyclene having 1-3 heteroatoms independently selected from nitrogen, oxygen, and sulfur, and 5- to 6-membered heteroarylene having 1-3 heteroatoms independently selected from nitrogen, oxygen, and sulfur;
X³ is hydrogen or an optionally substituted ring selected from 3- to 7-membered saturated or partially unsaturated carbocyclyl, phenyl, 3- to 7-membered saturated or partially unsaturated heterocyclyl having 1-3 heteroatoms independently selected from nitrogen, oxygen, and sulfur, or 5- to 6-membered heteroaryl having 1-3 heteroatoms independently selected from nitrogen, oxygen, and sulfur;
each R is independently hydrogen or an optionally substituted C₁-C₆ aliphatic group;
Z¹ is a covalent bond or —O—;
Z² is an optionally substituted group selected from 4- to 12-membered saturated or partially unsaturated carbocyclyl, phenyl, 1-adamantyl, and 2-adamantyl;
Z³ is hydrogen, or an optionally substituted group selected from C₁-C₁₀ aliphatic, and 4- to 12-membered saturated or partially unsaturated carbocyclyl; and
d is 0, 1, 2, 3, 4, 5, or 6; provided that when L³ is a covalent bond, then R¹ must be 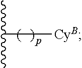
F) an amino lipid of Formula (If): 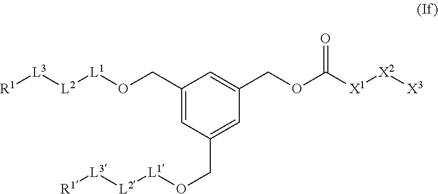
or a pharmaceutically acceptable salt thereof, wherein:
each L¹ and L^{1′} is independently-C(O)— or —C(O)O—;
each L² and L^{2′} is independently a covalent bond, an optionally substituted bivalent saturated or unsaturated, straight or branched C₁-C₁₂ hydrocarbon chain, or 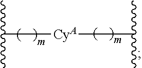
each Cy^{A} is independently an optionally substituted ring selected from phenylene or a 3- to 7-membered saturated or partially unsaturated carbocyclene;
each m is independently 0, 1, or 2;
each L³ and L^{3′} is independently a covalent bond, —C(O)O—, —OC(O)—, —O—, or —OC(O)O—;
each R¹ and R^{1′} is independently an optionally substituted group selected from a saturated or unsaturated, straight or branched C₁-C₂₀ hydrocarbon chain, wherein 1-3 methylene units are optionally and independently replaced with —O— or —NR—, a 3- to 12-membered saturated or partially unsaturated carbocyclic ring, 1-adamantyl, 2-adamantyl, sterolyl, phenyl, and 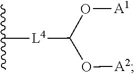
each L⁴ is independently a bivalent saturated or unsaturated, straight or branched C₁-C₆ hydrocarbon chain;
each A¹ and A² is independently an optionally substituted C₁-C₂₀ aliphatic or —L⁵-R⁵;
or A¹ and A², together with their intervening atoms, may form an optionally substituted ring: 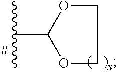
where
x is selected from 1 or 2; and
#represents the point of attachment to L⁴;
each L⁵ is independently a bivalent saturated or unsaturated, straight or branched C₁-C₂₀ hydrocarbon chain, wherein 1-3 methylene units are optionally and independently replaced with —O— or —NR—;
each R⁵ is independently an optionally substituted group selected from a 5- to 10-membered aryl ring and a 3- to 8-membered carbocyclic ring;
X¹ is a covalent bond, —O—, or —NR—;
X² is a covalent bond or an optionally substituted, bivalent saturated or unsaturated, straight or branched, C₁-C₁₂ hydrocarbon chain, wherein 1-3 methylene units are optionally and independently replaced with —O— or —NR—;
X³ is hydrogen or —Cy^{B};
Cy^{B} is an optionally substituted ring selected from 3- to 7-membered saturated or partially unsaturated carbocyclyl, phenyl, 3- to 7-membered saturated or partially unsaturated heterocyclyl having 1-3 heteroatoms independently selected from nitrogen, oxygen, and sulfur, and 5- to 6-membered heteroaryl having 1-3 heteroatoms independently selected from nitrogen, oxygen, and sulfur; and
each R is independently hydrogen or an optionally substituted C₁-C₆ aliphatic group;
provided that when X³ is hydrogen, at least one of R¹ or R^{1′} is 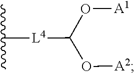
or
G) an amino lipid of Formula (Ig): 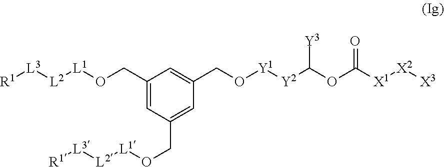
or a pharmaceutically acceptable salt thereof, wherein:
each of L¹ and L^{1′} is independently a covalent bond, —C(O)—, or —OC(O)—;
each of L² and L^{2′} is independently a covalent bond, an optionally substituted bivalent saturated or unsaturated, straight or branched C₁-C₁₂ hydrocarbon chain, or 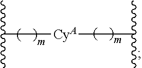
each Cy^{A} is independently an optionally substituted ring selected from phenylene or 3- to 7-membered saturated or partially unsaturated carbocyclene;
each m is independently 0, 1, or 2;
each of L³ and L^{3′} is independently a covalent bond, —O—, —C(O)O—, —OC(O)—, or —OC(O)O—;
each of R¹ and R^{1′} is independently an optionally substituted group selected from saturated or unsaturated, straight or branched C₁-C₂₀ hydrocarbon chain wherein 1-3 methylene units are optionally and independently replaced with —O— or —NR—, a 3- to 7-membered saturated or partially unsaturated carbocyclic ring, 1-adamantyl, 2-adamantyl, sterolyl, phenyl, or 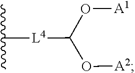
each L⁴ is independently a bivalent saturated or unsaturated, straight or branched C₁-C₂₀ hydrocarbon chain;
each A¹ and A² is independently an optionally substituted C₁-C₂₀ aliphatic or —L⁵-R⁵,
or A¹ and A², together with their intervening atoms, may form an optionally substituted ring: 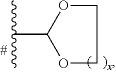
wherein
x is selected from 1 or 2; and
#represents the point of attachment to L⁴;
each L⁵ is independently a bivalent saturated or unsaturated, straight or branched C₁-C₂₀ hydrocarbon chain, wherein 1-3 methylene units are optionally and independently replaced with —O— or —NR—;
each R⁵ is independently an optionally substituted group selected from a 6- to 10-membered aryl ring or a 3- to 8-membered carbocyclic ring;
Y¹ is a covalent bond, —C(O)—, or —C(O)O—;
Y² is a bivalent saturated or unsaturated, straight or branched C₁-C₆ hydrocarbon chain, wherein 1-2 methylene units are optionally and independently replaced with cyclopropylene, —O—, or —NR—;
Y³ is an optionally substituted group selected from saturated or unsaturated, straight or branched C₁-C₁₄ hydrocarbon chain, wherein 1-3 methylene units are optionally and independently replaced with —O— or —NR—, a 3- to 7-membered saturated or partially unsaturated carbocyclic ring, 1-adamantyl, 2-adamantyl, or phenyl;
X¹ is a covalent bond, —O—, or —NR—;
X² is an optionally substituted bivalent saturated or unsaturated, straight or branched C₁-C₁₂ hydrocarbon chain, wherein 1-3 methylene units are optionally and independently replaced with —O—, —NR—, or —Cy^{B}—;
each Cy^{B} is independently an optionally substituted ring selected from 3- to 7-membered saturated or partially unsaturated carbocyclene, phenylene, 3- to 7-membered heterocyclene having 1-3 heteroatoms independently selected from nitrogen, oxygen, and sulfur, or 5- to 6-membered heteroarylene having 1-3 heteroatoms independently selected from nitrogen, oxygen, and sulfur;
X³ is hydrogen or an optionally substituted ring selected from 3- to 7-membered saturated or partially unsaturated carbocyclyl, phenyl, 3- to 7-membered heterocyclyl having 1-3 heteroatoms independently selected from nitrogen, oxygen, and sulfur, or 5- to 6-membered heteroaryl having 1-3 heteroatoms independently selected from nitrogen, oxygen, and sulfur; and
each R is independently hydrogen or an optionally substituted C₁-C₆ aliphatic group.

2. The LNP of claim 1, wherein the amino lipid is a compound of Formula A′: 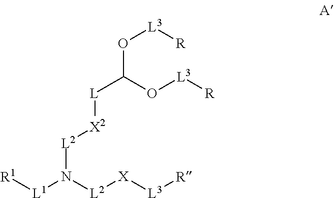
or its N-oxide, or a pharmaceutically acceptable salt thereof, wherein
L¹ is absent, C₁-6 alkylenyl, or C₂-6 heteroalkylenyl;
each L² is independently optionally substituted C₂₋₁₅ alkylenyl, or optionally substituted C₃₋₁₅ heteroalkylenyl;
L is C₁-10 alkylenyl, or C₂-10 heteroalkylenyl;
X² is —OC(O)—, —C(O)O—, or —OC(O)O—;
X is absent, —OC(O)—, —C(O)O—, or —OC(O)O—;
R″ is hydrogen, 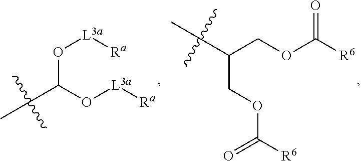
or an optionally substituted group selected from C₆-20 aliphatic, 3- to 12-membered cycloaliphatic, 7- to 12-membered bridged bicyclic comprising 0-4 heteroatoms independently selected from nitrogen, oxygen, or sulfur, 1-adamantyl, 2-adamantyl, sterolyl, and phenyl;
each of R and R^{a} is independently hydrogen, or an optionally substituted group selected from C₆-20 aliphatic, 3- to 12-membered cycloaliphatic, 7- to 12-membered bridged bicyclic comprising 0-4 heteroatoms independently selected from nitrogen, oxygen, or sulfur, 1-adamantyl, 2-adamantyl, sterolyl, and phenyl
each of L³ and L^{3a} is independently absent, optionally substituted C₁₋₁₀ alkylenyl, or optionally substituted C₂₋₁₀ heteroalkylenyl;
R¹ is hydrogen, optionally substituted phenyl, optionally substituted 3- to 7-membered cycloaliphatic, optionally substituted 3- to 7-membered heterocyclyl comprising 1-3 heteroatoms independently selected from nitrogen, oxygen, and sulfur, optionally substituted 5- to 6-membered monocyclic heteroaryl comprising 1-4 heteroatoms independently selected from nitrogen, oxygen, and sulfur, optionally substituted 8- to 10-membered bicyclic heteroaryl comprising 1-4 heteroatoms independently selected from nitrogen, oxygen, and sulfur, —OR², —C(O)OR², —C(O) SR², —OC(O)R², —OC(O)OR², —CN, —N(R²)₂, —C(O)N(R²)₂, —S(O)₂N(R²)₂, —NR²C(O)R², —OC(O)N(R²)₂, —N(R²)C(O)OR², —NR²S(O)₂R², —NR²C(O)N(R²)₂, —NR²C(S)N(R²)₂, —NR²C(NR²)N(R²)₂, —NR²C(CHR²)N(R²)₂, —N(OR²)C(O)R², —N(OR²) S(O)₂R², —N(OR²)C(O)OR², —N(OR²)C(O)N(R²)₂,
—N(OR²)C(S)N(R²)₂, —N(OR²)C(NR²)N(R²)₂, —N(OR²)C(CHR²)N(R²)₂, —C(NR²)N(R²)₂, —C(NR²)R², —C(O)N(R²)OR², —C(R²)N(R²)₂C(O)OR², —CR² (R³)₂, —OP(O) (OR²)₂, or —P(O) (OR²)₂; or
R¹ is 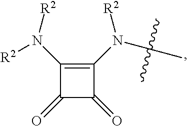
or a ring selected from 3- to 7-membered cycloaliphatic and 3- to 7-membered heterocyclyl comprising 1-3 heteroatoms independently selected from nitrogen, oxygen, and sulfur, wherein the cycloaliphatic or heterocyclyl ring is optionally substituted with 1-4 R² or R³ groups;
each R² is independently hydrogen, oxo, —CN, —NO₂, —OR⁴, —S(O)₂R⁴, —S(O)₂N(R⁴)₂, —(CH₂) n-R⁴, or an optionally substituted group selected from C₁-6 aliphatic, phenyl, 3- to 7-membered cycloaliphatic, 5- to 6-membered monocyclic heteroaryl comprising 1-4 heteroatoms independently selected from nitrogen, oxygen, and sulfur, and 3- to 7-membered heterocyclyl comprising 1-3 heteroatoms independently selected from nitrogen, oxygen, and sulfur; or
two occurrences of R², taken together with the atom(s) to which they are attached, form optionally substituted 4- to 7-membered heterocyclyl comprising 0-1 additional heteroatom selected from nitrogen, oxygen, and sulfur;
each R³ is independently-(CH₂) n-R⁴; or
two occurrences of R³, taken together with the atom(s) to which they are attached, form optionally substituted 5- to 6-membered heterocyclyl comprising 0-1 additional heteroatom selected from nitrogen, oxygen, and sulfur;
each R⁴ is independently hydrogen, —OR⁵, —N(R⁵)₂, —OC(O)R⁵, —OC(O)OR⁵, —CN, —C(O)N(R⁵)₂,
—NR⁵C(O)R⁵, —OC(O)N(R⁵)₂, —N(R⁵)C(O)OR⁵, —NR⁵S(O)₂R⁵, —NR⁵C(O)N(R⁵)₂, —NR⁵C(S)N(R⁵)₂, —NR⁵C(NR⁵)N(R⁵)₂, or 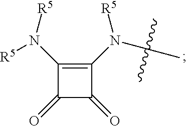
each R⁵ is independently hydrogen, or optionally substituted C₁₋₆ aliphatic; or
two occurrences of R⁵, taken together with the atom(s) to which they are attached, form optionally substituted 4- to 7-membered heterocyclyl comprising 0-1 additional heteroatom selected from nitrogen, oxygen, and sulfur;
each R⁶ is independently C₄-12 aliphatic; and
each n is independently 0 to 4.

3. The LNP of claim 2, wherein the amino lipid is a compound of Formula III-a-i: 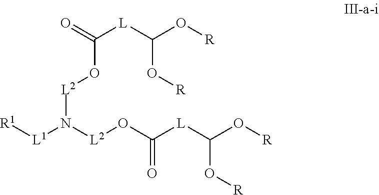
or its N-oxide, or a pharmaceutically acceptable salt thereof,
wherein each of R, R¹, L, L¹, and L² is as defined for Formula A′ of claim 2.

4. The LNP of claim 2, wherein the amino lipid is a compound of the formula BLP8-4: 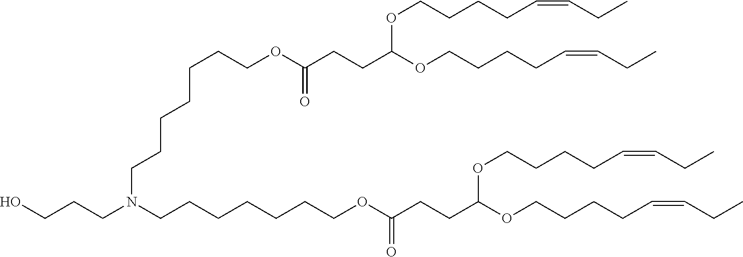 [SMILES: CC/C=C\CCCCOC(CCC(=O)OCCCCCCCN(CCCO)CCCCCCCOC(=O)CCC(OCCCC/C=C\CC)OCCCC/C=C\CC)OCCCC/C=C\CC]
or pharmaceutically acceptable salt thereof.

5. The LNP of claim 1, wherein the amino lipid is a compound of Formula I: 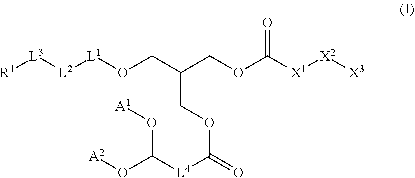
or a pharmaceutically acceptable salt thereof, wherein:
L¹ is a covalent bond, —C(O)—, or —OC(O)—;
L² is a covalent bond, an optionally substituted bivalent saturated or unsaturated, straight or branched C₁-C₁₂ hydrocarbon chain, or 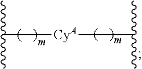
Cy^{A} is an optionally substituted ring selected from phenylene and 3- to 7-membered saturated or partially unsaturated carbocyclene;
each m is independently 0, 1, or 2;
L³ is a covalent bond, —C(O)—, —C(O)O—, —OC(O)—, —O—, or —OC(O)O—;
R¹ is 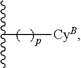
an optionally substituted saturated or unsaturated, straight or branched C₁-C₂₀ hydrocarbon chain wherein 1-3 methylene units are optionally and independently replaced with —O— or —NR—, or 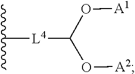
Cy^{B} is an optionally substituted ring selected from 3- to 12-membered saturated or partially unsaturated carbocyclyl, 1-adamantyl, 2-adamantyl, 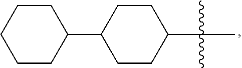 [SMILES: *C1CCC(C2CCCCC2)CC1]
sterolyl, and phenyl;
p is 0, 1, 2, or 3;
each L⁴ is independently a bivalent saturated or unsaturated, straight or branched C₁-C₆ hydrocarbon chain;
each A¹ and A² is independently an optionally substituted C₁-C₂₀ aliphatic or —L⁵-R⁵;
or A¹ and A², together with their intervening atoms, may form an optionally substituted 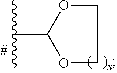
ring:
where
x is selected from 1 or 2; and
#represents the point of attachment to L⁴;
each L⁵ is independently a bivalent saturated or unsaturated, straight or branched C₁-C₂₀ hydrocarbon chain, wherein 1-3 methylene units are optionally and independently replaced with —O— or —NR—;
each R⁵ is independently an optionally substituted group selected from a 5- to 10-membered aryl ring or a 3- to 8-membered carbocyclic ring;
X¹ is a covalent bond, —O—, or —NR—;
X² is a covalent bond or an optionally substituted, bivalent saturated or unsaturated, straight or branched, C₁-C₁₂ hydrocarbon chain, wherein 1-3 methylene units are optionally and independently replaced with —O—, —NR—, or —Cy^{C}—;
Cy^{C} is an optionally substituted ring selected from 3- to 7-membered saturated or partially unsaturated carbocyclene, phenylene, 3- to 7-membered saturated or partially unsaturated heterocyclene having 1-3 heteroatoms independently selected from nitrogen, oxygen, and sulfur, and 5- to 6-membered heteroarylene having 1-3 heteroatoms independently selected from nitrogen, oxygen, and sulfur;
X³ is hydrogen or an optionally substituted ring selected from 3- to 7-membered saturated or partially unsaturated carbocyclyl, phenyl, 3- to 7-membered saturated or partially unsaturated heterocyclyl having 1-3 heteroatoms independently selected from nitrogen, oxygen, and sulfur, or 5- to 6-membered heteroaryl having 1-3 heteroatoms independently selected from nitrogen, oxygen, and sulfur; and
each R is independently hydrogen or an optionally substituted C₁-C₆ aliphatic group;
provided that when L³ is a covalent bond, then R¹ must be

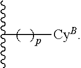

6. The LNP of claim 5, wherein the amino lipid is a compound of Formula (VIA): 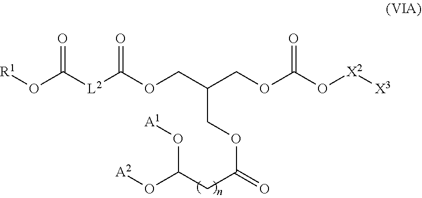
or a pharmaceutically acceptable salt thereof,
wherein n is 1, 2, 3 or 4, and L², R¹, A¹, A², X², and X³ are as defined for Formula I of claim 3.

7. The LNP of claim 5, wherein the amino lipid is a compound of the formula BLP4-71: 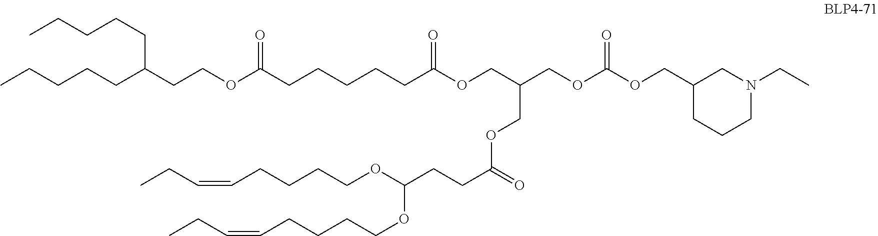 [SMILES: CC/C=C\CCCCOC(CCC(=O)OCC(COC(=O)CCCCCC(=O)OCCC(CCCCC)CCCCC)COC(=O)OCC1CCCN(CC)C1)OCCCC/C=C\CC]
or a pharmaceutically acceptable salt thereof.

8. The LNP of claim 1, wherein the LNP comprises an N: P ratio of between about 1:40 to about 1:1.

9. The LNP of claim 8, wherein the LNP comprises an N: P ratio of about 1:6.

10. The LNP of claim 1, wherein the guide polynucleotide comprises a scaffold sequence selected from the following:
GUUUUAGAGCUAGAAAUAGCAAGUUAAAAUAAGGCUAGUCCGUUAUCAACUUGAAAAAGUGG CACCGAGUCGGUGCmU*mU*mU*U (SEQ ID NO: 317);
mGUUUUAGmAmGmCmUmAGmAmAmAmUmAmGmCmAmAGUUmAAmAAmUAmAmGmGmCmUmAG UmCmCGUUAmUmCAAmCmUmUGmAmAmAmAmAmGmUmGGmCmAmCmCmGmAmGmUmCmGmGm UmGmCmU*mU*mU*mU (SEQ ID NO: 317), and
mG*U*U*U*U*A*G*mA*mG*mC*mU*mA*Gm*Am*Am*Am*Um*Am*Gm*Cm*Am*A*G*U *Um*A*A*mA*A*mU*A*mA*mG*mG*mC*mU*mA*G*U*mC*mC*G*U*U*A*mU*mC*A* A*mC*mU*mU*G*mA*mA*mA*mA*mA*mG*mU*mG*G*mC*mA*mC*mC*mG*mA*mG*mU *mC*mG*mG*mU*mG*mC*mU*mU*mU*mU (SEQ ID NO: 317), wherein A is adenosine, C is cytidine, G is guanosine, U is uridine, mA is 2′-O-methyladenosine, mC is 2′-O-methylcytidine, mG is 2′-O-methylguanosine, mU is 2′-O-methyluridine, and “*” indicates a phosphorothioate (PS) backbone linkage.

11. The LNP of claim 1, wherein the guide polynucleotide comprises 2-5 contiguous 2′-O-methylated nucleobases at the 3′ end and at the 5′ end.

12. The LNP of claim 1, wherein the guide polynucleotide comprises 2-5 contiguous nucleobases at the 3′ end and at the 5′ end that comprise phosphorothioate internucleotide linkages.

13. The LNP of claim 1, further comprising a polynucleotide encoding a base editor comprising a nucleic acid programmable DNA binding protein (napDNAbp) and a deaminase domain.

14. The LNP of claim 1, further comprising a polynucleotide encoding a nuclease active nucleic acid programmable DNA binding protein (napDNAbp).

15. A pharmaceutical composition comprising the LNP of claim 1.

16. A method of treating a disease or disorder, comprising administering to a subject in need thereof, the pharmaceutical composition of claim 15.

17. The method of claim 16, wherein the disease or disorder is hereditary transthyretin amyloidosis, cardiomyopathy, polyneuropathy or senile cardiac amyloidosis.

18. The method of claim 16, wherein the pharmaceutical composition is administered by a route selected from intravenous, intradermal, transdermal, intranasal, intramuscular, subcutaneous, transmucosal or oral.

19. The method of claim 16, wherein the LNP is delivered to liver.
